(19) United States
(12) Patent Application Publication
(10) Pub. No.: US 2023/0065368 A1
(43) Pub. Date: Mar. 2, 2023

(54) INHIBITING UBIQUITIN SPECIFIC PEPTIDASE 9X

(71) Applicant: FORMA Therapeutics, Inc., Watertown, MA (US)

(72) Inventors: Bruce FOLLOWS, Littleton, MA (US); Katherine J. KAYSER-BRICKER, Branford, CT (US); Adam Charles TALBOT, Watertown, MA (US); Scot MENTE, Watertown, MA (US); Tatiana SHELEKHIN, Ridgefield, CT (US); Anna ERICSSON, Shrewsbury, MA (US)

(21) Appl. No.: 17/277,500

(22) PCT Filed: Sep. 19, 2019
(86) PCT No.: PCT/US2019/051841

§ 371 Date: Mar. 18, 2021

Related U.S. Application Data
(60) Provisional application No. 62/733,595, filed on Sep. 19, 2018.

(51) Int. Cl. C07D 519/00; A61P 35/00; C07D 487/04

(52) U.S. Cl. CPC C07D 519/00; C07D 487/04; A61P 35/00

(57) ABSTRACT

The disclosure provides novel chemical compounds useful as inhibitors of ubiquitin specific peptidase 9X (USP9X). USP9X inhibiting compounds are useful in the treatment of disease and disorders associated with modulation of USP9X, such as cancer.

### CROSS-REFERENCE TO RELATED APPLICATION

This application claims priority to and the benefit of U.S. Provisional Application No. 62/733,595, filed Sep. 19, 2018, the entire contents of which are incorporated herein by reference.

### TECHNICAL FIELD

This disclosure relates to novel chemical compositions for inhibiting ubiquitin specific peptidase 9X.

### BACKGROUND

Deubiquitylating enzymes (DUBs) control a number of cellular processes, including the stability and function of a variety of oncoproteins, by reversing ubiquitination. Ubiquitin specific peptidase 9X (USP9X) is a member of the USP family of DUBs and is a key regulator of protein homeostasis for protein substrates including several that are known to be important in cancer. These include oncogenic or protumorigenic proteins and proteins involved in the anti-tumor immune response. These proteins can be important in tumor cells, immune cells, or other cells, such as stromal cells that play a role in cancer. Examples include MCL-1, survivin, ITCH, and CEP55. Overexpression and/or mutation of DUBs and their substrates have been correlated with cancer initiation and progression. USP9X has been suggested to be a negative prognostic factor for several oncology indications and may be associated with decreased overall survival in some cancer types (e.g., esophageal squamous cell carcinoma, non-small cell lung cancer, and multiple myeloma). Targeting USP9X can enhance an anti-tumor immune response through regulation of key maintenance proteins. Therefore, USP9X is a target for cancer drug development, particularly as a means to deplete oncoprotein substrates that have been labeled undruggable and/or through activation of the immune response. There is a need for compounds that inhibit USP9X and are useful for treating diseases and disorders associated with modulation of USP9X.

### SUMMARY

One aspect of this disclosure relates to compounds of Formula I.

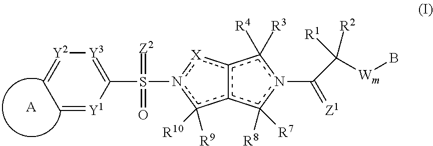

or a pharmaceutically acceptable salt thereof, wherein:
X is CR⁵SR⁶, CR⁵, NR⁵, or N, as valency permits;
dashed bonds are each independently a single or a double bond, as valency permits;
Y¹, Y², and Y³ are each independently N or CR^{a};
each R^{a} is independently —H, halogen, or —CN;
Ring A is a 5- to 6-membered aryl, 5- to 6-membered heteroaryl containing 1-4 heteroatoms independently selected from the group consisting of O, N, and S, 5- to 7-membered heterocyclyl containing 1-4 heteroatoms independently selected from the group consisting of O, N, and S, or 5- to 7-membered cycloalkyl,
   wherein each aryl, heteroaryl, heterocyclyl, or cycloalkyl is optionally substituted with one or more halogen, —C₁-C₆alkyl, —C₂-C₆alkenyl, —C₂-C₆alkynyl, oxo, or —C(O)R′;
Z¹ is O, S, or NR;
Z² is O or NR;
W is CR^{1′}R^{2′}, O, S, or NR;
m is 0 or 1;
R¹ and R² are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ⁿC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each cycloalkyl, cycloalkenyl, heterocyclyl, aryl, or heteroaryl is optionally substituted with one or more R^{e},
   wherein each alkyl, alkenyl, or alkynyl is optionally substituted with one or more halogen,
   wherein each heterocyclyl is 3- to 14-membered and contains 1-4 heteroatoms independently selected from the group consisting of O, N, and S, and wherein the heterocyclyl does not contain an O in the γ-position relative to C(═Z¹), and
   wherein each heteroaryl is 5- to 14-membered and contains 1-4 heteroatoms independently selected from the group consisting of O, N, and S;
or R¹ and R² combine with the carbon to which they are attached to form oxo, a C₃-C₈cycloalkyl, or a 3- to 8-membered heterocyclyl containing 1-4 heteroatoms independently selected from the group consisting of O, N, and S, and wherein the heterocyclyl does not contain an O in the γ-position relative to C(═Z¹), and
   wherein each cycloalkyl or heterocyclyl is optionally substituted with one or more R^{e};
R^{1′} and R^{2′} are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each alkyl, alkenyl, alkynyl, cycloalkyl, cycloalkenyl, heterocyclyl, aryl, or heteroaryl is optionally substituted with one or more R^{e},
   wherein each heterocyclyl is 3- to 14-membered and contains 1-4 heteroatoms independently selected from the group consisting of O, N, and S, and
   wherein each heteroaryl is 5- to 14-membered and contains 1-4 heteroatoms independently selected from the group consisting of O, N, and S;
or R^{1′} and R^{2′} combine with the carbon to which they are attached to form oxo, a C₃-C₆cycloalkyl, or a 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e};
or R¹ and R^{1′} combine with the carbons to which they are attached to form a C₃-C₈cycloalkyl or 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e};
R^{b} and R^{c} are each independently selected from the group consisting of —H, halogen, and —C₁-C₆alkyl;
each n is independently 0, 1, 2, 3, or 4;
each R^{e} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S, and wherein —OR of R^{e} does not result in an O in the γ-position relative to C(═Z¹), wherein each alkyl, alkenyl, alkynyl, cycloalkyl, cycloalkenyl, heterocyclyl, aryl, or heteroaryl is
   optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
B is a monocyclic or bicyclic 3- to 14-membered ring,
   wherein the ring is saturated, fully or partially unsaturated, or aromatic, and
   wherein the ring contains 0-4 heteroatoms independently selected from the group consisting of O, N, and S,
   wherein the ring is optionally substituted with one or more R^{d}, and
   when m is 0 and the ring is saturated or partially unsaturated, then the ring does not contain an O in the γ-position relative to C(═Z¹);
each R^{d} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, —C₆-C₁₄aryl, and 5- to 14-membered heteroaryl containing 1-4 heteroatoms independently selected from the group consisting of O, N, and S, wherein each alkyl, alkenyl, alkynyl, cycloalkyl, cycloalkenyl, heterocyclyl, aryl, or heteroaryl is
   optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
each R³, R⁴, R⁵, R⁶, R⁷, R⁸, R⁹, and R¹⁰ is independently —H, —C₁-C₆alkyl, —C₃-C₈cycloalkyl, or 3- to 8-membered heterocyclyl containing 1-4 heteroatoms independently selected from the group consisting of O, N, and S,
   wherein each alkyl, cycloalkyl, or heterocyclyl is optionally substituted with one or more halogen, oxo, —C₁-C₆alkyl, —C₂-C₆alkenyl, —C₂-C₆alkynyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₃-C₈cycloalkyl, or 3- to 8-membered heterocyclyl containing 1-4 heteroatoms independently selected from the group consisting of O, N, and S, and
   wherein R³, R⁷, and R⁹ are each independently present or absent, as valency permits;
or R³ and R⁴, R⁵ and R⁶, R⁷ and R⁸, R⁹ and R¹⁰, or combinations thereof, combine with the carbon to which they are attached to form an oxo, C₃-C₈cycloalkyl, or 3- to 8-membered heterocyclyl containing 1-4 heteroatoms independently selected from the group consisting of O, N, and S;
each R is independently selected from the group consisting of —H, —OH, —O(C₁-C₆alkyl), —NH₂, —NH(C₁-C₆alkyl), —N(C₁-C₆alkyl)₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —O—C₁-C₆alkyl, —NH(C₁-C₆alkyl), —N(C₁-C₆alkyl)₂, —C₁-C₆alkyl optionally substituted with one or more oxo or —OH, —C₂-C₆alkenyl, —C₂-C₆alkynyl, —C₃-C₁₂cycloalkyl, or 3- to 8-membered heterocyclyl containing 1-4 heteroatoms independently selected from the group consisting of O, N, and S; and
each R′ is independently selected from the group consisting of —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —C₁-C₆alkyl optionally substituted with one or more oxo or —OH, —C₂-C₆alkenyl, —C₂-C₆alkynyl, —O—C₁-C₆alkyl, —NH(C₁-C₆alkyl), or —N(C₁-C₆alkyl)₂.

Another aspect of this invention relates to compounds of Formula I that are USP9X Inhibitors. Unless otherwise indicated, a “USP9X Inhibitor” as used herein refers to a compound of Formula I having one or more of the following characteristics when tested in the Biochemical Assay of Example 1: (i) an IC₅₀ value of ≤2 μM and >0.001 μM; (ii) an IC₅₀ value of ≤0.2 μM and >0.001 μM; and (iii) an IC₅₀ value of ≤0.05 μM and >0.001 μM. In some embodiments, a USP9X Inhibitor is a compound of Formula I having an IC₅₀ value of ≤2 μM and >0.001 μM when tested in the Biochemical Assay of Example 1. In some embodiments, a USP9X Inhibitor is a compound of Formula I having an IC₅₀ value of ≤0.2 μM and >0.001 μM when tested in the Biochemical Assay of Example 1. In some embodiments, a USP9X Inhibitor is a compound of Formula I having an IC₅₀ value of ≤0.05 μM and >0.001 μM when tested in the Biochemical Assay of Example 1.

In some embodiments, a USP9X Inhibitor is provided, wherein Ring A contains at least one oxygen atom (e.g., dioxane, tetrahydropyran, morpholine, or furan). In some embodiments, a USP9X Inhibitor is provided, wherein Ring A does not contain a nitrogen atom (e.g., dioxane, tetrahydropyran, or furan). In some embodiments, a USP9X Inhibitor is provided, wherein Ring A contains at least one oxygen atom and does not contain a nitrogen atom (e.g., dioxane, tetrahydropyran, or furan). In some embodiments, a USP9X Inhibitor is provided, wherein Y¹, Y², and Y³ are not further substituted (e.g., N or CH).

In some embodiments, a USP9X Inhibitor is provided, wherein one of R¹ and R² is —H, resulting in a disubstituted α-carbon. In some embodiments, one of R¹ and R² is —H, and the other is a small group, i.e., a group small enough so that the compound is a USP9X Inhibitor, (e.g., —OH, —NHC(O)Me, or —CH₂NHMe). In some embodiments, one of R¹ and R² is —H, and the other is not a bulky group, i.e., the other is not a bulky group such that the compound is a USP9X Inhibitor. In some embodiments, one of R¹ and R² is —H, and the other is a neutral group, i.e., a group that is natural so that the compound is a USP9X Inhibitor, (e.g., —OH or —NHC(O)Me). In some embodiments, one of R¹ and R² is —H, and the other is a neutral, hydrogen bond-donating group, i.e., a group that is neutral and hydrogen-bonding donating so that the compound is a USP9X Inhibitor, (e.g., —OH or —NHC(O)Me). In some embodiments, one of R¹ and R² is —H, and the other is a basic group, i.e., a group that is basic enough so that the compound is a USP9X Inhibitor, (e.g., —CH₂NHMe, —CH₂azetidinyl, —CH₂pyrrolidinyl, or —CH₂morpholinyl). In some embodiments, a USP9X Inhibitor is provided, wherein one of R¹ and R² are —H, and the other is a basic group with a pKa of the conjugate acid of approximately 8 or approximately 8.5 (e.g., —CH₂NHMe, —CH₂azetidinyl, —CH₂pyrrolidinyl, or —CH₂morpholinyl).

In some embodiments, a USP9X Inhibitor is provided, wherein B is a monocyclic aryl (e.g., phenyl). In some embodiments, a USP9X Inhibitor is provided, wherein B is a monocyclic aryl substituted in the meta position with R^{d} (e.g., fluoro, chloro, methyl, ethyl, —CHF₂, —CF₃, cyclopropyl, oxetanyl, piperazinyl, N-methylpiperazinyl, 2-(difluoromethyl)piperazinyl, 4-cyclopropylpiperazinyl, morpholinyl, 2-methyloctahydropyrrolo[3,4-c]pyrrolyl, 2-oxa-7-azaspiro[3.5]nonanyl, 2-methyl-1,2,3,4,5,6-hexahydropyrrolo[3,4-c]-pyrrolyl, 3a-fluoro-2-methyloctahydropyrrolo[3,4-c]pyrrolyl, 9,9-difluoro-3,7-diazabicyclo[3.3.1]nonanyl, —OMe, —OCHF₂, —O(cyclopropyl), —O(cyclobutyl), —O(N-methylazetidinyl), or —N(Me)(CH₂CH₂OH)). In some embodiments, B is a monocyclic aryl substituted in the meta position with a large R^{d} group, i.e., a group that is large enough so that the compound is a USP9X Inhibitor, (e.g., piperazinyl, N-methylpiperazinyl, 2-(difluoromethyl)piperazinyl, 4-cyclopropylpiperazinyl, morpholinyl, 2-methyloctahydropyrrolo[3,4-c]pyrrolyl, 2-oxa-7-azaspiro[3.5]nonanyl, 2-methyl-1,2,3,4,5,6-hexahydropyrrolo[3,4-c]-pyrrolyl, 3a-fluoro-2-methyloctahydropyrrolo[3,4-c]pyrrolyl, or 9,9-difluoro-3,7-diazabicyclo[3.3.1]nonanyl). In some embodiments, B is a monocyclic aryl substituted in the meta position with a basic R^{d} group, i.e., a group that is basic enough so that the compound is a USP9X Inhibitor, (e.g., piperazinyl, N-methylpiperazinyl, 2-(difluoromethyl)piperazinyl, 4-cyclopropylpiperazinyl, morpholinyl, 2-methyloctahydropyrrolo[3,4-c]pyrrolyl, 2-oxa-7-azaspiro[3.5]nonanyl, or —N(Me)(CH₂CH₂OH)). In some embodiments, B is a monocyclic aryl substituted in the para position with a small R^{d} group, i.e., a group that is small enough so that the compound is a USP9X Inhibitor, (e.g., fluoro, chloro, or —OMe). In some embodiments, B is a monocyclic aryl with —H in the para position. In some embodiments, B is a monocyclic aryl substituted in the ortho position with a small R^{d} group, i.e., a group that is small enough so that the compound is a USP9X Inhibitor, (e.g., fluoro, chloro, or —OMe). In some embodiments, B is a monocyclic aryl with —H in the ortho position. In some embodiments, B is a bicyclic ring, wherein at least one of the rings is an aromatic ring.

In some embodiments, a USP9X Inhibitor is provided, wherein Ring A contains at least one oxygen atom; one of R¹ and R² is —H; and B is a monocyclic aryl. In some embodiments, a USP9X Inhibitor is provided, wherein Ring A contains at least one oxygen atom; one of R¹ and R² is —H, and the other is a small group, i.e., a group small enough so that the compound is a USP9X Inhibitor; and B is a monocyclic aryl. In some embodiments, a USP9X Inhibitor is provided, wherein Ring A contains at least one oxygen atom; one of R¹ and R² is —H, and the other is a neutral group, i.e., a group that is neutral so that the compound is a USP9X Inhibitor; and B is a monocyclic aryl. In some embodiments, a USP9X Inhibitor is provided, wherein Ring A contains at least one oxygen atom; one of R¹ and R² is —H, and the other is a neutral, hydrogen bond-donating group, i.e., a group that is neutral and hydrogen-bonding donating so that the compound is a USP9X Inhibitor; and B is a monocyclic aryl. In some embodiments, a USP9X Inhibitor is provided, wherein Ring A contains at least one oxygen atom; one of R¹ and R² is —H, and the other is a basic group, i.e., a group that is basic enough so that the compound is a USP9X Inhibitor; and B is a monocyclic aryl. In some embodiments, a USP9X Inhibitor is provided, wherein Ring A contains at least one oxygen atom; one of R¹ and R² are —H, and the other is a basic group with a pKa of the conjugate acid of approximately 8 or approximately 8.5; and B is a monocyclic aryl.

In some embodiments, a USP9X Inhibitor is provided, wherein Ring A contains at least one oxygen atom; one of R¹ and R² is —H; and B is a monocyclic aryl substituted in the meta position with R^{d}. In some embodiments, a USP9X Inhibitor is provided, wherein Ring A contains at least one oxygen atom; one of R¹ and R² is —H, and the other is a small group, i.e., a group small enough so that the compound is a USP9X Inhibitor; and B is a monocyclic aryl substituted in the meta position with R^{d}. In some embodiments, a USP9X Inhibitor is provided, wherein Ring A contains at least one oxygen atom; one of R¹ and R² is —H, and the other is a neutral group, i.e., a group that is neutral so that the compound is a USP9X Inhibitor; and B is a monocyclic aryl substituted in the meta position with R^{d}. In some embodiments, a USP9X Inhibitor is provided, wherein Ring A contains at least one oxygen atom; one of R¹ and R² is —H, and the other is a neutral, hydrogen bond-donating group, i.e., a group that is neutral and hydrogen-bonding donating so that the compound is a USP9X Inhibitor; and B is a monocyclic aryl substituted in the meta position with R^{d}. In some embodiments, a USP9X Inhibitor is provided, wherein Ring A contains at least one oxygen atom; one of R¹ and R² is —H, and the other is a basic group, i.e., a group that is basic enough so that the compound is a USP9X Inhibitor; and B is a monocyclic aryl substituted in the meta position with R^{d}. In some embodiments, a USP9X Inhibitor is provided, wherein Ring A contains at least one oxygen atom; one of R¹ and R² are —H, and the other is a basic group with a pKa of the conjugate acid of approximately 8 or approximately 8.5; and B is a monocyclic aryl substituted in the meta position with R^{d}.

In some embodiments, a USP9X Inhibitor is provided, wherein Ring A contains at least one oxygen atom; one of R¹ and R² is —H; and B is a bicyclic ring, wherein at least one of the rings is an aromatic ring. In some embodiments, a USP9X Inhibitor is provided, wherein Ring A contains at least one oxygen atom; one of R¹ and R² is —H, and the other is a small group, i.e., a group small enough so that the compound is a USP9X Inhibitor; and B is a bicyclic ring, wherein at least one of the rings is an aromatic ring. In some embodiments, a USP9X Inhibitor is provided, wherein Ring A contains at least one oxygen atom; one of R¹ and R² is —H, and the other is a neutral group, i.e., a group that is neutral so that the compound is a USP9X Inhibitor; and B is a bicyclic ring, wherein at least one of the rings is an aromatic ring. In some embodiments, a USP9X Inhibitor is provided, wherein Ring A contains at least one oxygen atom; one of R¹ and R² is —H, and the other is a neutral, hydrogen bond-donating group, i.e., a group that is neutral and hydrogen-bonding donating so that the compound is a USP9X Inhibitor; and B is a bicyclic ring, wherein at least one of the rings is an aromatic ring. In some embodiments, a USP9X Inhibitor is provided, wherein Ring A contains at least one oxygen atom; one of R¹ and R² is —H, and the other is a basic group, i.e., a group that is basic enough so that the compound is a USP9X Inhibitor; and B is a bicyclic ring, wherein at least one of the rings is an aromatic ring. In some embodiments, a USP9X Inhibitor is provided, wherein Ring A contains at least one oxygen atom; one of R¹ and R² are —H, and the other is a basic group with a pKa of the conjugate acid of approximately 8 or approximately 8.5; and B is a bicyclic ring, wherein at least one of the rings is an aromatic ring.

### DETAILED DESCRIPTION

One aspect of this invention relates to compounds of Formula I.

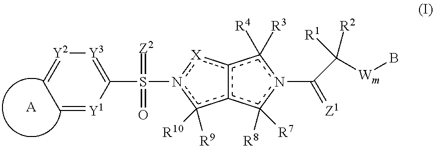

or a pharmaceutically acceptable salt thereof,
wherein X, dashed bonds, Y¹, Y², Y³, Ring A, B, Z¹, Z², W, m, R¹, R², R³, R⁴, R⁷, R⁸, R⁹, and R¹⁰ are as defined above for Formula I and described in classes and subclasses herein, both singly and in combination.

In some embodiments, compounds are provided that are compounds of Formula II:

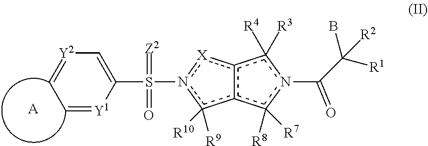

or a pharmaceutically acceptable salt thereof,
wherein X, dashed bonds, Y¹, Y², Z², B, R¹, R², R³, R⁴, R⁷, R⁸, R⁹, and R¹⁰ are as defined above and described in classes and subclasses herein, both singly and in combination, and
wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆alkyl.

In some embodiments, compounds are provided that are compounds of Formula II-a:

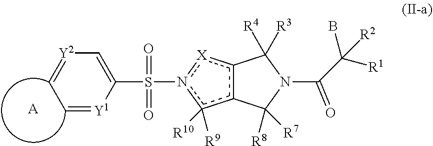

or a pharmaceutically acceptable salt thereof,
wherein X, dashed bonds, Y¹, Y², B, R¹, R², R³, R⁴, R⁷, R⁸, R⁹, and R¹⁰ are as defined above and described in classes and subclasses herein, both singly and in combination, and
wherein Ring A is a 5- to 6-membered heteroaryl containing 1-4 heteroatoms independently selected from the group consisting of O, N, and S, or 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆alkyl.

In some embodiments, compounds are provided that are compounds of Formula II-b:

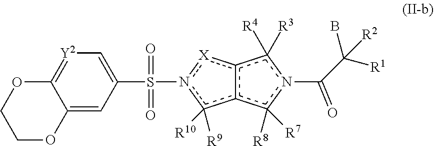

or a pharmaceutically acceptable salt thereof,
wherein X, dashed bonds, B, R¹, R², R³, R⁴, R⁷, R⁸, R⁹, and R¹⁰ are as defined above and described in classes and subclasses herein, both singly and in combination, and
wherein Y² is CH or N.

In some embodiments, compounds are provided that are compounds of Formula II-c:

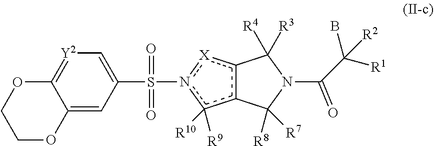

or a pharmaceutically acceptable salt thereof,
wherein X, dashed bonds, B, R¹, R², R³, R⁴, R⁷, R⁸, R⁹, and R¹⁰ are as defined above and described in classes and subclasses herein, both singly and in combination, and
wherein Y² is CH or N.

In some embodiments, compounds are provided that are compounds of Formula III:

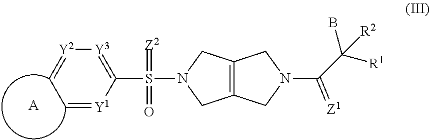

or a pharmaceutically acceptable salt thereof,
wherein Y¹, Y², Y³, Ring A, B, Z¹, Z², R¹, and R² are as defined above and described in classes and subclasses herein, both singly and in combination.

In some embodiments, compounds are provided that are compounds of Formula III-a:

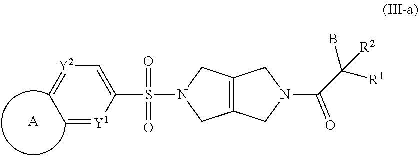

or a pharmaceutically acceptable salt thereof,
wherein Y¹, Y², R¹, R², and B are as defined above and described in classes and subclasses herein, both singly and in combination, and
wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆alkyl.

In some embodiments, compounds are provided that are compounds of Formula III-b:

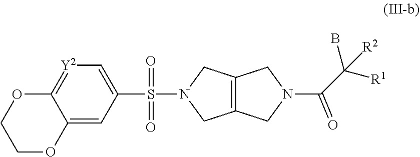

or a pharmaceutically acceptable salt thereof,
wherein B, R¹, and R² are as defined above, and
wherein Y² is CH or N.

In some embodiments, compounds are provided that are compounds of Formula III-c:

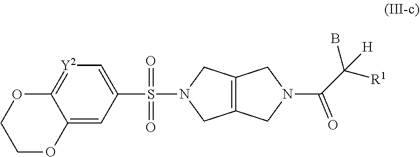

or a pharmaceutically acceptable salt thereof, wherein:
Y² is CH or N;
R¹ is —OH or —(CH₂)NHMe;
B is a phenyl ring or a bicyclic ring,
   wherein at least one of the rings in the bicyclic ring is a phenyl ring,
   wherein the phenyl ring or bicyclic ring contains 0-4 heteroatoms independently selected from the group consisting of O, N, and S, and
   wherein the phenyl ring or bicyclic ring is optionally substituted with one or more R^{d};
each R^{d} is independently selected from the group consisting of halogen, —C₁-C₆alkyl, and —OR; and
each R is independently —H, —C₁-C₆alkyl, or 3- to 8-membered heterocyclyl optionally substituted with —C₁-C₆alkyl.

In some embodiments, compounds of Formula III are provided, wherein:
Y¹, Y², and Y³ are each independently N or CR^{a};
R^{a} is —H or halogen;
Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S,
   wherein each heteroaryl or heterocyclyl is optionally substituted with one or more halogen or —C₁-C₆alkyl;
Z¹ is O or S;
Z² is O or NR;
R¹ and R² are each independently —H, halogen, —C₁-C₆alkyl, —(CR^{b}R^{c})ₙC₃-C₁₂cycloalkyl, —(CR^{b}R^{c})ₙheterocyclyl, —OR, —(CR^{b}R^{c})ₙNR₂, —(CR^{b}R^{c})ₙNRC(O)R′, —(CR^{b}R^{c})ₙNRS(O)₂R′, or —(CR^{b}R^{c})ₙNRC(O)NR₂,
   wherein each heterocyclyl is optionally substituted with one or more substituents selected from the group consisting of halogen, —OR, and oxo, and wherein —OR does not result in an O in the γ-position relative to C(═Z¹), and
   wherein each heterocyclyl is 3- to 14-membered and contains 1-4 heteroatoms independently selected from the group consisting of O, N, and S, and wherein the heterocyclyl does not contain an O in the γ-position relative to C(═Z¹);
or R¹ and R² combine with the carbon to which they are attached to form a C₃-C₈cycloalkyl or 3- to 8-membered heterocyclyl containing 1-4 heteroatoms independently selected from the group consisting of O, N and S, and wherein the heterocyclyl does not contain an O in the γ-position relative to C(═Z¹);
R^{b} and R^{c} are each independently —H;
n is 0, 1, or 2;
B is a monocyclic or bicyclic 3- to 14-membered ring,
   wherein the ring is saturated, fully or partially unsaturated, or aromatic, and
   wherein the ring contains 0-4 heteroatoms independently selected from the group consisting of O, N, and S,
   wherein the ring is optionally substituted with one or more R^{d},
   and when the ring is saturated or partially unsaturated, then the ring does not contain an O in the γ-position relative to C(═Z¹);
each R^{d} is independently selected from the group consisting of halogen, oxo, —OR, —NR₂, —C(O)NR₂, —C₁-C₆alkyl, —C₃-C₁₂cycloalkyl, 3- to 14-membered heterocyclyl containing 1-4 heteroatoms independently selected from the group consisting of O, N, and S, and C₆-C₁₄aryl,
wherein each alkyl, heterocyclyl, or aryl is optionally substituted with one or more substituents selected from the group consisting of halogen, —C₁-C₆alkyl optionally substituted with one or more halogen, or —C₃-C₁₂cycloalkyl;
each R is independently selected from the group consisting of —H, —C₁-C₆alkyl, —C₃-C₁₂cycloalkyl, and 3- to 14-membered heterocyclyl containing 1-4 heteroatoms independently selected from the group consisting of O, N, and S,
   wherein each alkyl or heterocyclyl is optionally substituted with one or more halogen, —O—C₁-C₆alkyl, —NH—C₁-C₆alkyl, —N(C₁-C₆alkyl)₂, —C₁-C₆alkyl optionally substituted with —OH, —C₃-C₁₂cycloalkyl, or 3- to 8-membered heterocyclyl containing 1-4 heteroatoms independently selected from the group consisting of O, N, and S; and
each R′ is —C₁-C₆alkyl.

In some embodiments, compounds of Formula III are provided, wherein:

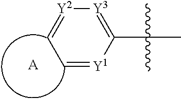

is selected from the group consisting of:

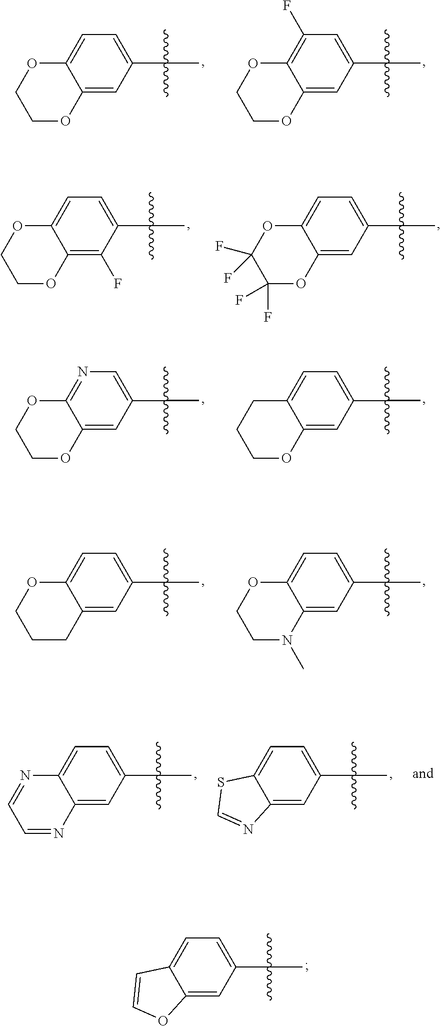

Z¹ is O or S;
Z² is O or NR;
R¹ and R² are each independently —H, fluoro, methyl, —(CH₂)ₙcyclopropyl, —(CH₂)ₙazetidinyl, —(CH₂)ₙpyrrolidinyl, —(CH₂)ₙpyrrolidinonyl, —(CH₂)ₙmorpholinyl, —OR, —(CH₂)ₙNR₂, —(CH₂)ₙNHC(O)R′, —(CH₂)ₙNHS(O)₂R′, or —(CH₂)ₙNHC(O)NHR,
   wherein each azetidinyl is optionally substituted with one or more substituents selected from the group consisting of fluoro and —OH, and wherein —OH does not result in an O in the γ-position relative to C(═Z¹);
or R¹ and R² combine with the carbon to which they are attached to form a cyclopropyl or pyrrolidinyl;
n is 0, 1, or 2;
B is a monocyclic or bicyclic 3- to 14-membered ring selected from the group consisting of:

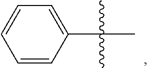 [SMILES: *c1ccccc1]

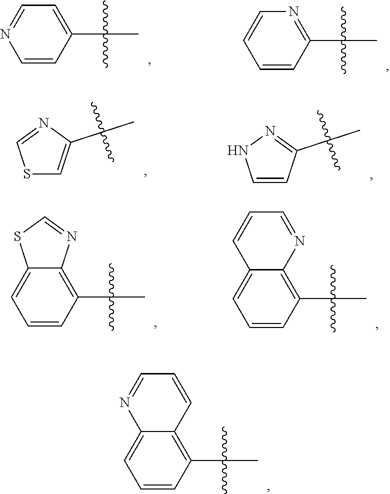

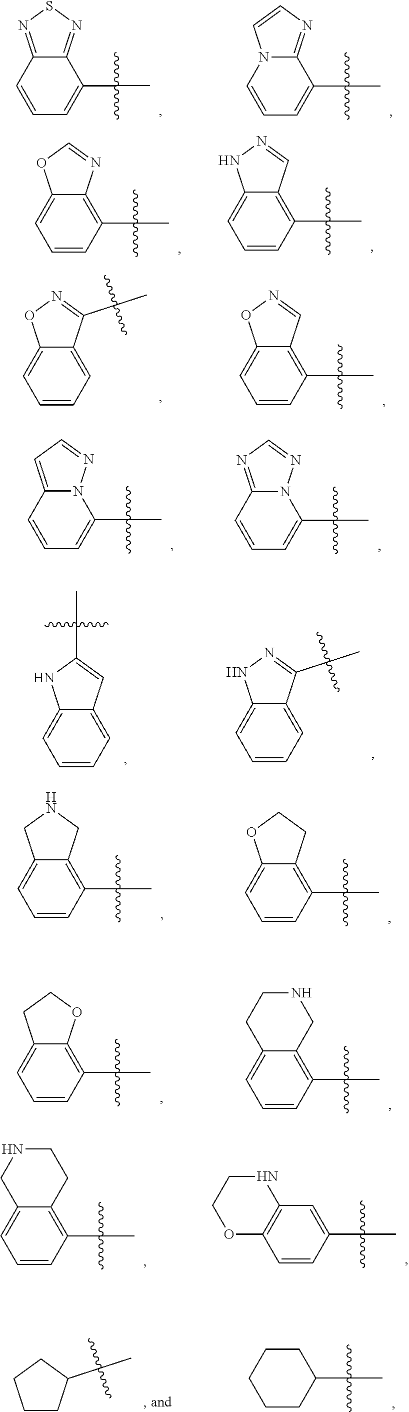

wherein the ring is optionally substituted with one or more R^{d};
each R^{d} is independently selected from the group consisting of fluoro, chloro, methyl, ethyl, —CHF₂, —CF₃, —OR, —NR₂, —C(O)NMe₂, cyclopropyl, oxetanyl, piperazinyl, N-methylpiperazinyl, 2-(difluoromethyl)piperazinyl, 4-cyclopropylpiperazinyl, morpholinyl, 2-methyloctahydropyrrolo[3,4-c]pyrrolyl, 2-oxa-7-azaspiro[3.5]nonanyl, 2-methyl-1,2,3,4,5,6-hexahydropyrrolo[3,4-c]pyrrolyl, 3a-fluoro-2-methyloctahydropyrrolo[3,4-c]pyrrolyl, 9,9-difluoro-3,7-diazabicyclo[3.3.1]nonanyl, and 4-fluorophenyl;
each R is independently selected from the group consisting of —H, methyl, ethyl, isopropyl, —CHF₂, cyclopropyl, cyclobutyl, azetidinyl, and oxetanyl,
   wherein each methyl, ethyl, or azetidinyl is optionally substituted with one or more fluoro, methyl, —(CH₂)₂OH, cyclobutyl, cyclopentyl, tetrahydropyranyl, —OMe, —NHMe, and —NMe₂; and
each R′ is methyl.

In some embodiments, compounds are provided that are compounds of Formula IV:

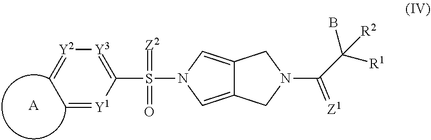

or a pharmaceutically acceptable salt thereof,
wherein Y¹, Y², Y³, Ring A, B, Z¹, Z², R¹, and R² are as defined above and described in classes and subclasses herein, both singly and in combination.

In some embodiments, compounds are provided that are compounds of Formula IV-a:

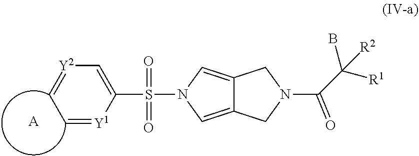

or a pharmaceutically acceptable salt thereof,
wherein Y¹, Y², B, R¹, and R² are as defined above and described in classes and subclasses herein, both singly and in combination, and
wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆alkyl.

In some embodiments, compounds are provided that are compounds of Formula IV-b:

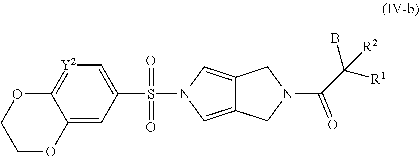

or a pharmaceutically acceptable salt thereof,
wherein B, R¹, and R² are as defined above and described in classes and subclasses herein, both singly and in combination, and
wherein Y² is CH or N.

In some embodiments, compounds of Formula IV are provided, wherein:
Y¹, Y², and Y³ are each independently N or CR^{a};
R^{a} is —H;
Ring A is a 6-membered heterocyclyl containing 0-4 heteroatoms independently selected from the group consisting of O, N, and S;
Z¹ is O;
Z² is O;
R¹ and R² are each independently —H, —OR, or —(CR^{b}R^{c})ₙNR₂;
R^{b} and R^{c} are each independently —H;
n is 1;
B is a monocyclic or bicyclic 3- to 14-membered ring,
   wherein the ring is partially unsaturated or aromatic, and
   wherein the ring contains 0-4 heteroatoms independently selected from the group consisting of O, N, and S, and
   wherein the ring is optionally substituted with one or more R^{d};
each R^{d} is independently selected from the group consisting of halogen, —C₁-C₆alkyl, and —OR; and
each R is independently selected from the group consisting of —H and —C₁-C₆alkyl.

In some embodiments, compounds of Formula IV are provided, wherein:

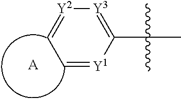

is selected from the group consisting of:

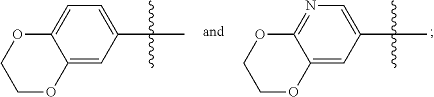 [SMILES: *c1ccc2c(c1)OCCO2 ; *c1cnc2c(c1)OCCO2]

### Z¹ is O;

### Z² is O;

R¹ and R² are each independently —H, —OH, or —CH₂NHMe;
B is a monocyclic or bicyclic 3- to 14-membered ring selected from the group consisting of:

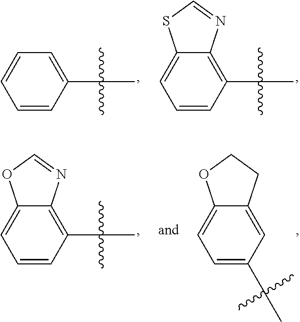 [SMILES: *c1ccccc1 ; *c1cccc2scnc12 ; *c1cccc2ocnc12 ; *c1ccc2c(c1)CCO2]

wherein the ring is optionally substituted with one or more R^{d}; and

each R^{d} is independently selected from the group consisting of fluoro, chloro, methyl, and —OMe.

In some embodiments, compounds are provided that are compounds of Formula V:

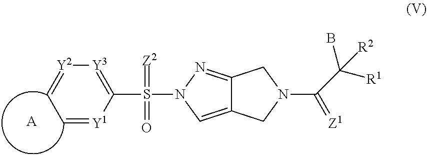

or a pharmaceutically acceptable salt thereof,
wherein Y¹, Y², Y³, Ring A, B, Z¹, Z², R¹, and R² are as defined above and described in classes and subclasses herein, both singly and in combination.

In some embodiments, compounds are provided that are compounds of Formula V-a:

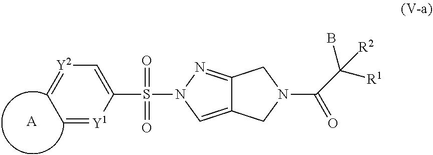

or a pharmaceutically acceptable salt thereof,
wherein Y¹, Y², B, R¹, and R² are as defined above and described in classes and subclasses herein, both singly and in combination,
wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆alkyl.

In some embodiments, compounds are provided that are compounds of Formula V-b:

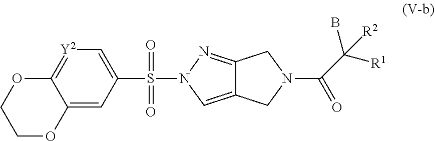

or a pharmaceutically acceptable salt thereof,
wherein B, R¹, and R² are as defined above and described in classes and subclasses herein, both singly and in combination,
wherein Y² is CH or N.

In some embodiments, compounds of Formula V are provided, wherein:
Y¹, Y², and Y³ are each CR^{a};
each R^{a} is —H;
Ring A is a 6-membered heterocyclyl containing 1-4 heteroatoms independently selected from the group consisting of O, N, and S;
Z¹ is O;
Z² is O;
R¹ and R² are each independently —H or —OR;
B is a monocyclic or bicyclic 3- to 14-membered ring,
   wherein the ring is partially unsaturated or aromatic, and
   wherein the ring contains 0-4 heteroatoms independently selected from the group consisting of O and N, and
   wherein the ring is optionally substituted with one or more —OR;
and
each R is independently selected from the group consisting of —H and —C₁-C₆alkyl.

In some embodiments, compounds of Formula V are provided, wherein:

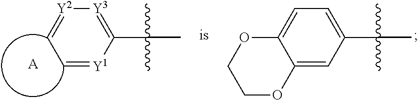

### Z¹ is O;

### Z² is O;

R¹ and R² are each independently —H or —OH;
B is a monocyclic or bicyclic 3- to 14-membered ring selected from the group consisting of:

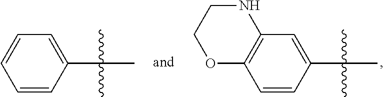 [SMILES: *c1ccccc1 ; *c1ccc2c(c1)NCCO2]

wherein the ring is optionally substituted with one or more —OMe.

In some embodiments, compounds are provided that are compounds of Formula I-a:

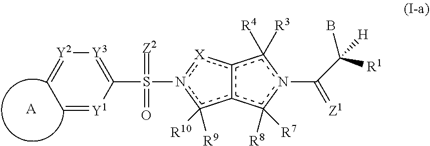

or a pharmaceutically acceptable salt thereof,
wherein X, dashed bonds, Y¹, Y², Y³, Ring A, B, Z¹, Z², R¹, R³, R⁴, R⁷, R⁸, R⁹, and R¹⁰ are as defined above and described in classes and subclasses herein, both singly and in combination.

In some embodiments, compounds are provided that are compounds of Formula I-b:

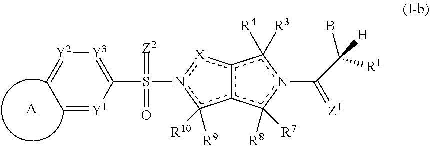

or a pharmaceutically acceptable salt thereof,
wherein X, dashed bonds, Y¹, Y², Y³, Ring A, B, Z¹, Z², R¹, R³, R⁴, R⁷, R⁸, R⁹, and R¹⁰ are as defined above and described in classes and subclasses herein, both singly and in combination.

In some embodiments, compounds are provided that are compounds of Formula III-d:

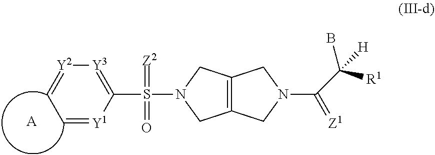

or a pharmaceutically acceptable salt thereof,
wherein Y¹, Y², Y³, Ring A, B, Z¹, Z², and R¹ are as defined above and described in classes and subclasses herein, both singly and in combination.

In some embodiments, compounds are provided that are compounds of Formula III-e:

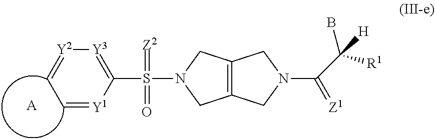

or a pharmaceutically acceptable salt thereof,
wherein Y¹, Y², Y³, Ring A, B, Z¹, Z², and R¹ are as defined above and described in classes and subclasses herein, both singly and in combination.

In some embodiments of Formulas I, I-a, I-b, II, II-a, II-b, and II-c, at least one dashed bond is a double bond. In some embodiments, one dashed bond is a double bond. In some embodiments, two dashed bonds are double bonds.

In some embodiments of Formulas I, I-a, I-b, II, II-a, II-b, and II-c, X is CR⁵R⁶, CR⁵, or N. In some embodiments, X is CR⁵R⁶. In some embodiments, X is CR⁵. In some embodiments, X is NR⁵. In some embodiments, X is N. In some embodiments, X is CH₂. In some embodiments, X is CH. In some embodiments, X is NH.

In some embodiments of Formulas I, I-a, I-b, II, II-a, III, III-a, III-d, III-e, IV, IV-a, V, and V-a, Y¹, Y², and Y³ are each independently CR^{a}. In some embodiments, Y¹, Y², and Y³ are each CH. In some embodiments, at least one of Y¹, Y², and Y³ is N. In some embodiments, at least one of Y¹ and Y² is N. In some embodiments, Y¹ is CR^{a}. In some embodiments, Y¹ is N. In some embodiments, Y² is CR^{a}. In some embodiments, Y² is N. In some embodiments, Y³ is CR^{a}. In some embodiments, Y³ is N.

In some embodiments of Formulas I, I-a, I-b, II, II-a, III, III-a, III-d, III-e, IV, IV-a, V, and V-a, each R^{a} is independently —H, —F, —Cl, or —CN. In some embodiments, each R^{a} is —H. In some embodiments, each R^{a} is —F. In some embodiments, each R^{a} is —Cl. In some embodiments, each R^{a} is —CN.

In some embodiments of Formulas I, I-a, I-b, II, II-a, III, III-a, III-d, III-e, IV, IV-a, V, and V-a, Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl is optionally substituted with one or more halogen or —C₁-C₆ alkyl. In some embodiments, Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆ alkyl. In some embodiments, Ring A is a 5- to 6-membered heterocyclyl containing 1-4 heteroatoms independently selected from the group consisting of O, N, and S, wherein heterocyclyl contains at least one oxygen atom and is optionally substituted with one or more halogen or —C₁-C₆alkyl. In some embodiments, Ring A is an unsubstituted 5- to 6-membered heterocyclyl containing 1-4 heteroatoms independently selected from the group consisting of O, N, and S and containing at least one oxygen atom. In some embodiments, Ring A is an unsubstituted 5- to 6-membered heteroaryl containing 1-4 heteroatoms independently selected from the group consisting of O, N, and S and containing at least one oxygen atom.

In some embodiments of Formulas I, I-a, I-b, II, II-a, III, III-a, III-d, III-e, IV, IV-a, V, and V-a,

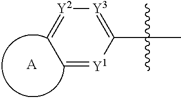

is selected from the group consisting of:

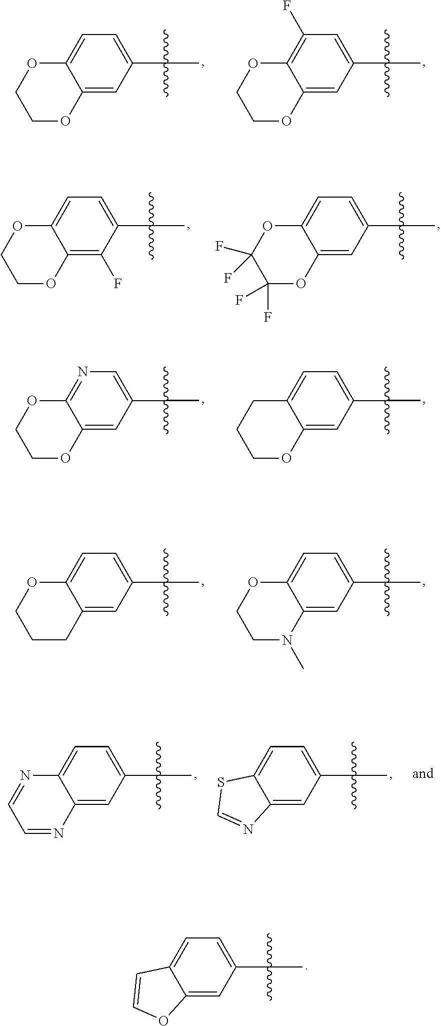

In some embodiments,

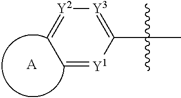

is selected from the group consisting of:

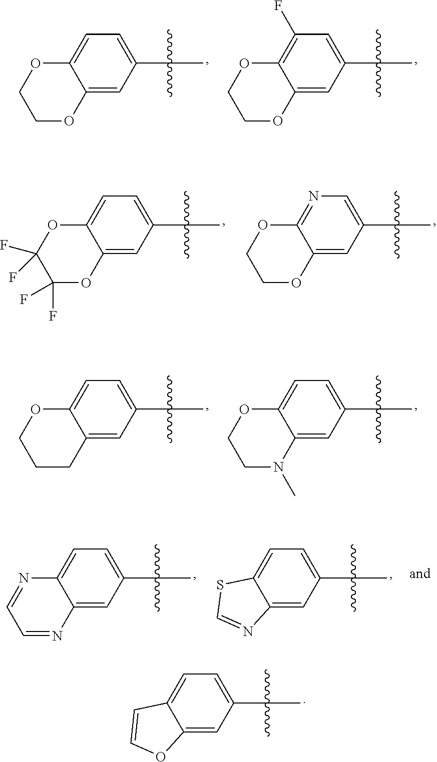

In some embodiments,

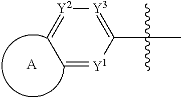

is selected from the group consisting of:

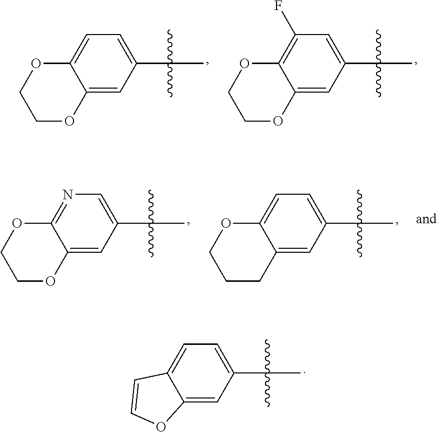 [SMILES: *c1ccc2c(c1)OCCO2 ; *c1cc(F)c2c(c1)OCCO2 ; *c1cnc2c(c1)OCCO2 ; *c1ccc2c(c1)CCCO2 ; *c1ccc2ccoc2c1]

In some embodiments,

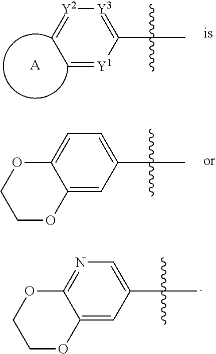

In some embodiments,

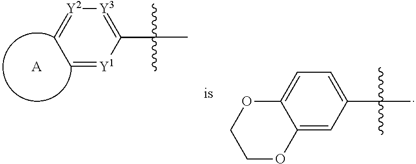

In some embodiments,

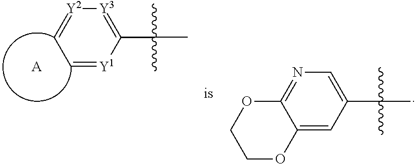

In some embodiments of Formulas I, I-a, I-b, III, III-d, III-e, IV, and V, Z¹ is O or S. In some embodiments, Z¹ is O. In some embodiments, Z¹ is S. In some embodiments, Z¹ is NR. In some embodiments, Z¹ is NH, NOH, or NNH₂.

In some embodiments of Formulas I, I-a, I-b, II, III, III-d, III-e, IV, and V, Z² is O or NH. In some embodiments, Z² is O. In some embodiments, Z² is NR. In some embodiments, Z² is NH.

In some embodiments of Formula I,W is CR^{1′}R^{2′}. In some embodiments, W is CH₂. In some embodiments, W is O, S, or NR. In some embodiments, W is O. In some embodiments, W is S. In some embodiments, W is NR (e.g., NH).

In some embodiments of Formulas I, I-a, I-b, II, II-a, II-b, II-c, III, III-a, III-b, III-d, III-e, IV, IV-a, IV-b, V, V-a, and V-b, R¹ and R² are each independently selected from the group consisting of —H, halogen (e.g., fluoro), —C₁-C₆alkyl (e.g., methyl), —(CR^{b}R^{c})ₙC₃-C₁₂cycloalkyl (e.g., —(CH₂)ₙcyclopropyl), —(CR^{b}R^{c})ₙheterocyclyl (e.g., —(CH₂)ₙazetidinyl, —(CH₂)ₙpyrrolidinyl, —(CH₂)ₙpyrrolidinonyl, or —(CH₂)ₙmorpholinyl), —OR, —(CR^{b}R^{c})ₙNR₂ (e.g., —(CH₂)ₙNR₂), —(CR^{b}R^{c})ₙNRC(O)R′ (e.g., —(CH₂)ₙNHC(O)R′), —(CR^{b}R^{c})ₙNRS(O)₂R′ (e.g., —(CH₂)ₙNHS(O)₂R′), or —(CR^{b}R^{c})ₙNRC(O)NR₂ (e.g., —(CH₂)ₙNHC(O)NHR),
wherein each heterocyclyl (e.g., azetidinyl) is optionally substituted with one or more substituents selected from the group consisting of halogen (e.g., fluoro), —OR (e.g., —OH), and oxo, and wherein —OR does not result in an O in the γ-position relative to C(═Z¹), and
wherein each heterocyclyl is 3- to 14-membered and contains 1-4 heteroatoms independently selected from the group consisting of O, N, and S and wherein the heterocyclyl does not contain an O in the γ-position relative to C(═Z¹); or
or R¹ and R² combine with the carbon to which they are attached to form a C₃-C₈cycloalkyl (e.g., cyclopropyl) or 3- to 8-membered heterocyclyl containing 1-4 heteroatoms independently selected from the group consisting of O, N, and S (e.g., pyrrolidinyl),
   wherein the heterocyclyl does not contain an O in the γ-position relative to C(═Z¹).

In some embodiments of Formulas I, I-a, I-b, II, II-a, II-b, II-c, III, III-a, III-b, III-d, III-e, IV, IV-a, IV-b, V, V-a, and V-b, R¹ and R² are each independently —H, halogen, —C₁-C₆alkyl, —(CR^{b}R^{c})ₙC₃-C₁₂cycloalkyl, —(CR^{b}R^{c})ₙheterocyclyl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, or —(CR^{b}R^{c})ₙNRC(O)OR, wherein each heterocyclyl is optionally substituted with one or more substituents selected from the group consisting of halogen, —OR, and oxo, and wherein —OR does not result in an O in the γ-position relative to C(═Z¹), wherein each heterocyclyl is 3- to 14-membered and contains 1-4 heteroatoms independently selected from the group consisting of O, N, and S, and wherein the heterocyclyl does not contain an O in the γ-position relative to C(═Z¹); or R¹ and R² combine with the carbon to which they are attached to form a C₃-C₈cycloalkyl or 3- to 8-membered heterocyclyl containing 1-4 heteroatoms independently selected from the group consisting of O, N, and S, wherein the heterocyclyl does not contain an O in the γ-position relative to C(═Z¹). In some embodiments, R¹ and R² are each independently selected from the group consisting of —H, halogen (e.g., fluoro), —C₁-C₆alkyl (e.g., methyl), —(CR^{b}R^{c})ₙheterocyclyl (e.g., —(CH₂)ₙazetidinyl or —(CH₂)ₙpyrrolidinyl), —OR, —(CR^{b}R^{c})ₙNR₂ (e.g., —(CH₂)ₙNR₂), —(CR^{b}R^{c})ₙNRC(O)R′ (e.g., —(CH₂)ₙNHC(O)R′), or —(CR^{b}R^{c})ₙNRC(O)NR₂ (e.g., —(CH₂)ₙNHC(O)NHR), wherein each heterocyclyl (e.g., azetidinyl) is optionally substituted with one or more halogen (e.g., fluoro), and wherein each heterocyclyl is 3- to 14-membered and contains 1-4 heteroatoms independently selected from the group consisting of O, N, and S, and wherein the heterocyclyl does not contain an O in the γ-position relative to C(═Z¹); or R¹ and R² combine with the carbon to which they are attached to form a 3- to 8-membered heterocyclyl containing 1-4 heteroatoms independently selected from O, N, and S (e.g., pyrrolidinyl), wherein the heterocyclyl does not contain an O in the γ-position relative to C(═Z¹). In some embodiments, R¹ and R² are each independently —H, —OR, —(CR^{b}R^{c})ₙNR₂, or —(CR^{b}R^{c})ₙNRC(O)R′. In some embodiments, R¹ and R² are each independently —H, —OR, —CH₂NR₂, or —CH₂NRC(O)R′. In some embodiments, R¹ and R² are each independently —H, —OH, —CH₂NHMe, or —CH₂NHC(O)Me. In some embodiments, R¹ and R² are each independently —H, —OH, or —CH₂NHMe. In some embodiments, one of R¹ and R² is not —H. In some embodiments, R′ is —OH. In some embodiments, R² is —H.

In some embodiments of Formulas I, I-a, I-b, II, II-a, II-b, II-c, III, III-a, III-b, III-d, III-e, IV, IV-a, IV-b, V, V-a, and V-b, R^{1′} and R^{2′} are each independently selected from the group consisting of —H, halogen, —C₁-C₆alkyl, —(CR^{b}R^{c})ₙC₃-C₁₂cycloalkyl, —(CR^{b}R^{c})ₙheterocyclyl, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, or —(CR^{b}R^{c})ₙNRC(O)OR,
wherein each alkyl, cycloalkyl, or heterocyclyl is optionally substituted with one or more substituents selected from the group consisting of halogen, —C₁-C₆alkyl, —OR, and oxo, and
wherein each heterocyclyl is 3- to 14-membered and contains 1-4 heteroatoms independently selected from the group consisting of O, N, and S, and wherein the heterocyclyl does not contain an O in the γ-position relative to C(═Z¹);
or R^{1′} and R^{2′} combine with the carbon to which they are attached to form a C₃-C₈cycloalkyl or a 3- to 8-membered heterocyclyl containing 1-4 heteroatoms independently selected from the group consisting of O, N and S,
   wherein the heterocyclyl does not contain an O in the γ-position relative to C(═Z¹);
or R¹ and R^{1′} combine with the carbons to which they are attached to form a C₃-C₈cycloalkyl or a 3- to 8-membered heterocyclyl containing 1-4 heteroatoms independently selected from the group consisting of O, N, and S,
   wherein each cycloalkyl or heterocyclyl is optionally substituted with one or more substituents independently selected from the group consisting of halogen, —C₁-C₆alkyl, —OR, and oxo, and
   wherein the heterocyclyl does not contain an O in the γ-position relative to C(═Z¹).

In some embodiments of Formulas I, I-a, I-b, II, II-a, II-b, II-c, III, III-a, III-b, III-d, III-e, IV, IV-a, IV-b, V, V-a, and V-b, R^{1′} and R^{2′} are each independently selected from the group consisting of —H, halogen, —C₁-C₆alkyl, —(CR^{b}R^{c})ₙheterocyclyl, —(CR^{b}R^{c})ₙNR₂, —(CR^{b}R^{c})ₙNRC(O)R′, or —(CR^{b}R^{c})ₙNRC(O)NR₂, wherein each heterocyclyl is optionally substituted with one or more halogen, and wherein each heterocyclyl is 3- to 14-membered and contains 1-4 heteroatoms independently selected from the group consisting of O, N, and S, and wherein the heterocyclyl does not contain an O in the γ-position relative to C(═Z¹); or R^{1′} and R^{2′} combine with the carbon to which they are attached to form a 3- to 8-membered heterocyclyl containing 1-4 heteroatoms independently selected from O, N, and S, wherein the heterocyclyl does not contain an O in the γ-position relative to C(═Z¹). In some embodiments, R^{1′} and R^{2′} are each independently —H, —(CR^{b}R^{c})ₙNR₂, or —(CR^{b}R^{c})ₙNRC(O)R′. In some embodiments, R^{1′} and R^{2′} are each —H.

In some embodiments of Formulas I, I-a, I-b, II, II-a, II-b, II-c, III, III-a, III-b, III-d, III-e, IV, IV-a, IV-b, V, V-a, and V-b, R^{b} and R^{c} are each independently —H, —F, or —C₁-C₆alkyl. In some embodiments, R^{b} and R^{c} are each independently —H, —F, or methyl. In some embodiments, R^{b} and R^{c} are both —H. In some embodiments, R^{b} and R^{c} are both halogen. In some embodiments, R^{b} and R^{c} are both —C₁-C₆alkyl. In some embodiments, one of R^{b} and R^{c} is halogen. In some embodiments, one of R and R is —C₁-C₆alkyl (e.g., methyl). In some embodiments, one of R^{b} and R^{c} is —F.

In some embodiments of Formulas I, I-a, I-b, II, II-a, II-b, II-c, III, III-a, III-b, III-d, III-e, IV, IV-a, IV-b, V, V-a, and V-b, each n is independently 0, 1, or 2. In some embodiments, each n is 0. In some embodiments, each n is 1. In some embodiments, each n is 2.

In some embodiments of Formula I, m is 0. In some embodiments, m is 1.

In some embodiments of Formulas I, I-a, I-b, II, II-a, II-b, II-c, III, III-a, III-b, III-d, III-e, IV, IV-a, IV-b, V, V-a, and V-b:

### B is:

(i) a monocyclic 3- to 8-membered ring, comprising a C₃-C₈cycloalkyl, 3- to 8-membered heterocyclyl, phenyl, or 5- to 8-membered heteroaryl ring,
wherein the ring contains 0-4 heteroatoms independently selected from the group consisting of O, N, and S,
wherein the ring is optionally substituted with one or more R^{d}, and
wherein the ring comprising a 3- to 8-membered heterocyclyl does not contain an O in the γ-position relative to C(═Z); or
(ii) a bicyclic 6- to 14-membered ring, comprising a C₃-C₁₀cycloalkyl, 3- to 11-membered heterocyclyl, phenyl, or 5- to 11-membered heteroaryl ring,
wherein the ring is fused to an aromatic, saturated, or partially unsaturated 3- to 8-membered carbocyclic or heterocyclic ring,
wherein the ring contains 0-4 heteroatoms independently selected from the group consisting of O, N, and S,
wherein the ring is optionally substituted with one or more R^{d}, and
wherein the ring comprising a 3- to 11-membered heterocyclyl does not contain an O in the γ-position relative to C(═Z).

In some embodiments of Formulas I, I-a, I-b, II, II-a, II-b, II-c, III, III-a, III-b, III-d, III-e, IV, IV-a, IV-b, V, V-a, and V-b, B is a phenyl ring or a bicyclic ring, wherein at least one of the rings in the bicyclic ring is a phenyl ring, wherein the phenyl ring or bicyclic ring contains 0-4 heteroatoms independently selected from the group consisting of O, N, and S, and wherein the phenyl ring or bicyclic ring is optionally substituted with one or more R^{d}. In some embodiments, B is a phenyl ring optionally substituted with one or more R^{d}. In some embodiments, B is a phenyl ring optionally substituted with one or more R^{d} and is fused to an aromatic, saturated, or partially unsaturated 5- to 8-membered carbocyclic or heterocyclic ring. In some embodiments, B is a phenyl ring optionally substituted with one or more R^{d} and is fused to a saturated or partially unsaturated 5- to 8-membered heterocyclic ring. In some embodiments, B is a monocyclic or bicyclic heteroaryl ring, wherein the ring contains 1-4 heteroatoms independently selected from the group consisting of O, N, and S, and wherein the ring is optionally substituted with one or more R^{d}.

In some embodiments, B is selected from the group consisting of:

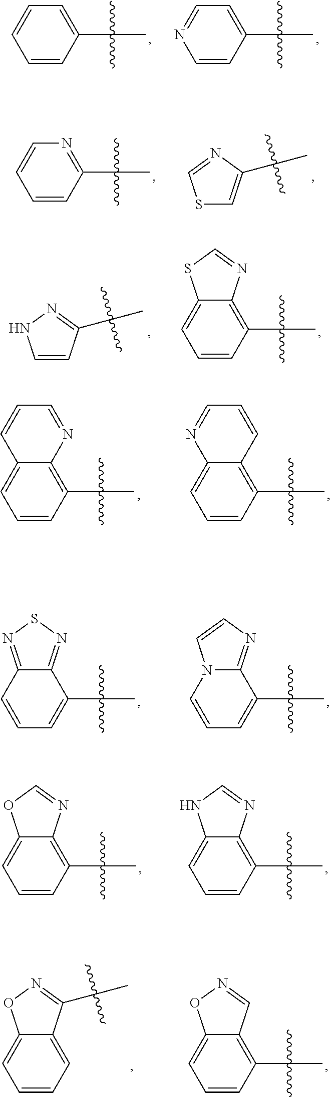

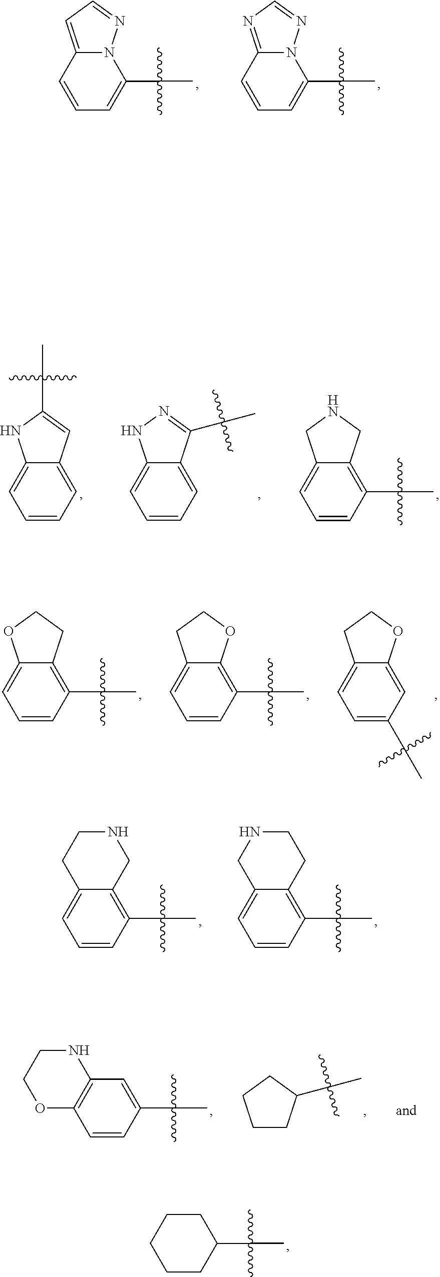

wherein the ring is optionally substituted with one or more R^{d}.

In some embodiments, B is selected from the group consisting of:

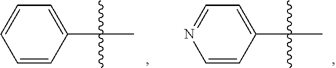 [SMILES: *c1ccccc1 ; *c1ccncc1]

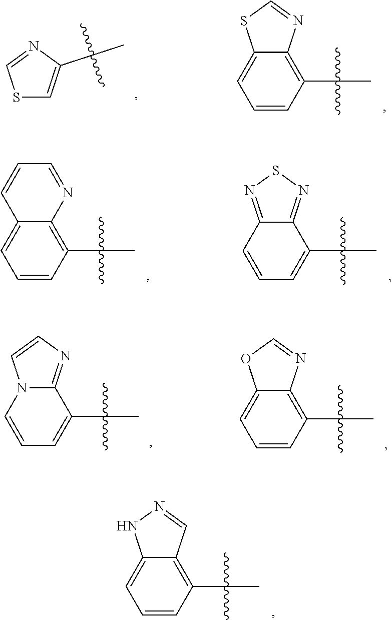

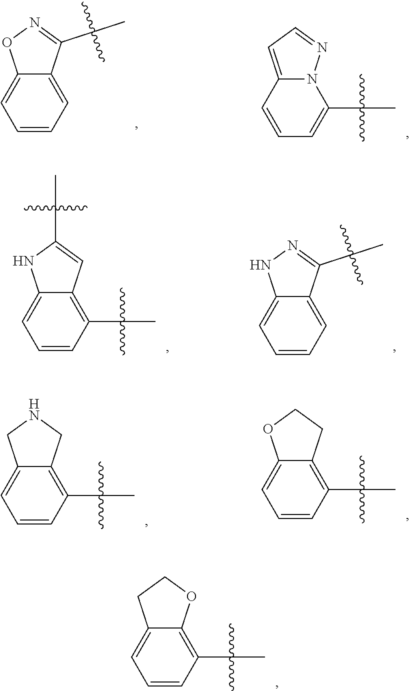

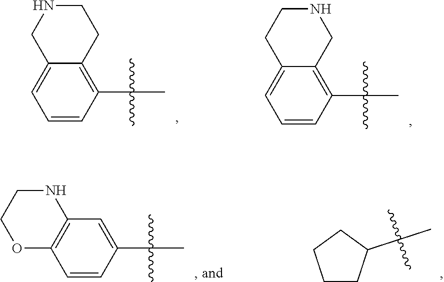 [SMILES: *c1cccc2c1CCNC2 ; *c1cccc2c1CNCC2 ; *c1ccc2c(c1)NCCO2 ; *C1CCCC1]

wherein the ring is optionally substituted with one or more R^{d}.

In some embodiments, B is selected from the group consisting of:

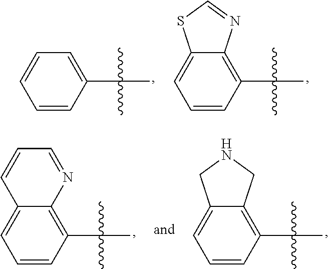

wherein the ring is optionally substituted with one or more R^{d}.

In some embodiments of Formulas I, I-a, I-b, II, II-a, II-b, II-c, III, III-a, III-b, III-d, III-e, IV, IV-a, IV-b, V, V-a, and V-b, each R^{d} is independently selected from the group consisting of halogen (e.g., fluoro or chloro), —OR (e.g., —OMe, —OCHF₂, —O(CH₂)₂NMe₂, —O(cyclopropyl), or —O(cyclobutyl)), —NR₂ (e.g., —N(Me)(CH₂CH₂OMe)), —C(O)NR₂ (e.g., —C(O)NMe₂), —C₁-C₆alkyl (e.g., methyl, ethyl, —CHF₂, or —CF₃), —C₃-C₁₂cycloalkyl (e.g., cyclopropyl), 3- to 14-membered heterocyclyl containing 1-4 heteroatoms independently selected from the group consisting of O, N, and S (e.g., oxetanyl, piperazinyl, N-methylpiperazinyl, 2-(difluoromethyl)piperazinyl, 4-cyclopropylpiperazinyl, morpholinyl, 2-methyloctahydropyrrolo[3,4-c]pyrrolyl, 2-oxa-7-azaspiro[3.5]nonanyl, 2-methyl-1,2,3,4,5,6-hexahydropyrrolo[3,4-c]pyrrolyl, 3a-fluoro-2-methyloctahydropyrrolo[3,4-c]pyrrolyl, or 9,9-difluoro-3,7-diazabicyclo[3.3.1]nonanyl), and C₆-C₁₄aryl (e.g., 4-fluorophenyl, or phenyl, naphthyl, or anthracenyl), wherein each alkyl (e.g., methyl or ethyl), heterocyclyl (e.g., piperazinyl or octahydropyrrolo[3,4-c]pyrrolyl), or aryl (e.g., phenyl) is optionally substituted with one or more substituents selected from the group consisting of halogen (e.g., fluoro), —C₁-C₆alkyl (e.g., methyl) optionally substituted with one or more halogen (e.g., fluoro), or —C₃-C₁₂cycloalkyl (e.g., cyclopropyl). In some embodiments, each R^{d} is independently selected from the group consisting of halogen (e.g., fluoro or chloro), —OR (e.g., —OMe, —OCHF₂, —O(CH₂)₂NMe₂, —O(cyclopropyl), or —O(cyclobutyl)), —C₁-C₆alkyl (e.g., methyl, ethyl, —CHF₂, or —CF₃), —C₃-C₁₂cycloalkyl (e.g., cyclopropyl), and 3- to 14-membered heterocyclyl containing 1-4 heteroatoms independently selected from the group consisting of O, N, and S (e.g., oxetanyl, piperazinyl, N-methylpiperazinyl, 2-(difluoromethyl)piperazinyl, 4-cyclopropylpiperazinyl, morpholinyl, 2-methyloctahydropyrrolo[3,4-c]pyrrolyl, 2-oxa-7-azaspiro[3.5]nonanyl, 2-methyl-1,2,3,4,5,6-hexahydropyrrolo[3,4-c]pyrrolyl, 3a-fluoro-2-methyloctahydropyrrolo[3,4-c]pyrrolyl, or 9,9-difluoro-3,7-diazabicyclo[3.3.1]nonanyl). In some embodiments, each R^{d} is independently selected from the group consisting of halogen (e.g., fluoro or chloro), —C₁-C₆alkyl (e.g., methyl), and —OR (e.g., —OMe or —O(1-methylazetidinyl)).

In some embodiments of Formulas I, I-a, I-b, II, II-a, II-b, and II-c, each R³, R⁴, R⁵, R⁶, R⁷, R⁸, R⁹, and R¹⁰, if present, is —H.

In some embodiments of Formulas I, I-a, I-b, II, II-a, II-b, II-c, III, III-a, III-b, III-d, III-e, IV, IV-a, IV-b, V, V-a, and V-b, each R is independently selected from the group consisting of —H, —C₁-C₆alkyl (e.g., methyl, ethyl, or isopropyl), —C₃-C₁₂cycloalkyl (e.g., cyclopropyl or cyclobutyl), and 3- to 14-membered heterocyclyl containing 1-4 heteroatoms independently selected from the group consisting of O, N, and S (e.g., azetidinyl or oxetanyl), wherein each alkyl (e.g., methyl or ethyl) or heterocyclyl (e.g., azetidinyl) is optionally substituted with one or more halogen (e.g., fluoro), —O—C₁-C₆alkyl (e.g., —OMe), —NH—C₁-C₆alkyl (e.g., —NHMe), —N(C₁-C₆alkyl)₂ (e.g., —NMe₂), —C₁-C₆alkyl optionally substituted with —OH (e.g., methyl or —(CH₂)₂OH), —C₃-C₁₂cycloalkyl (e.g., cyclobutyl or cyclopentyl), or 3- to 8-membered heterocyclyl containing 1-4 heteroatoms independently selected from the group consisting of O, N, and S (e.g., 1-methylazetidinyl or tetrahydropyranyl). In some embodiments, each R is independently —H, —C₁-C₆alkyl (e.g., methyl), or 3- to 8-membered heterocyclyl optionally substituted with C₁-C₆alkyl (e.g., 1-methylazetidinyl). In some embodiments, each R is independently —H or methyl.

In some embodiments of Formulas I, I-a, I-b, II, II-a, II-b, II-c, III, III-a, III-b, III-d, III-e, IV, IV-a, IV-b, V, V-a, and V-b, each R′ is independently —C₁-C₆alkyl, —C₃-C₁₂cycloalkyl, or 3- to 14-membered heterocyclyl containing 1-4 heteroatoms independently selected from the group consisting of O, N, and S. In some embodiments, each R′ is independently —C₁-C₆alkyl (e.g., methyl).

Another aspect of the present disclosure is a compound selected from Table 1, or a pharmaceutically acceptable salt thereof.

TABLE 1

| | |
|---|---|
| 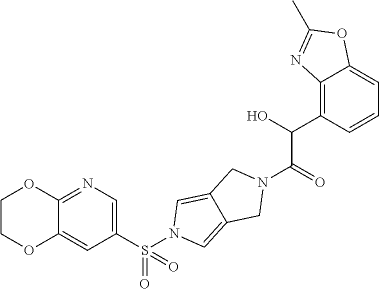 [SMILES: Cc1nc2c(C(O)C(=O)N3Cc4cn(S(=O)(=O)c5cnc6c(c5)OCCO6)cc4C3)cccc2o1] | Examples 94-1 and 94-2 |
| 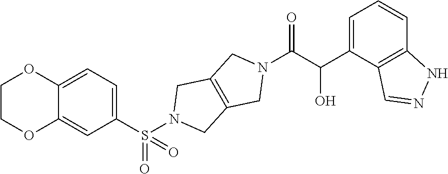 [SMILES: O=C(C(O)c1cccc2[nH]ncc12)N1CC2=C(C1)CN(S(=O)(=O)c1ccc3c(c1)OCCO3)C2] | Example 94-5 |
| 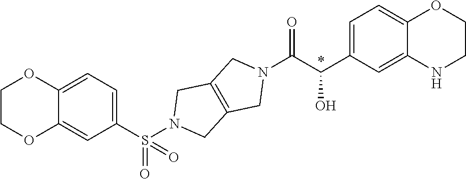 [SMILES: O=C([C@@H](O)c1ccc2c(c1)NCCO2)N1CC2=C(C1)CN(S(=O)(=O)c1ccc3c(c1)OCCO3)C2] | Example 94-6 |
| 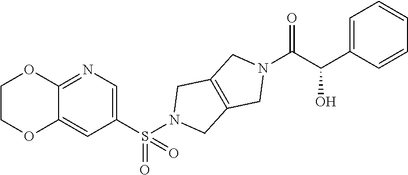 [SMILES: O=C([C@@H](O)c1ccccc1)N1CC2=C(C1)CN(S(=O)(=O)c1cnc3c(c1)OCCO3)C2] | Example 94-7 |
| 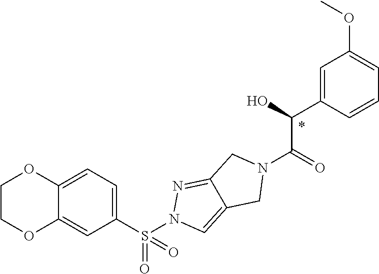 [SMILES: COc1cccc([C@H](O)C(=O)N2Cc3cn(S(=O)(=O)c4ccc5c(c4)OCCO5)nc3C2)c1] | Example 94-8 |
| 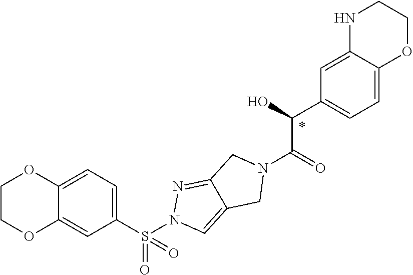 [SMILES: O=C([C@@H](O)c1ccc2c(c1)NCCO2)N1Cc2cn(S(=O)(=O)c3ccc4c(c3)OCCO4)nc2C1] | Example 94-9 |
| 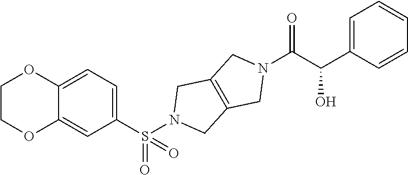 [SMILES: O=C([C@@H](O)c1ccccc1)N1CC2=C(C1)CN(S(=O)(=O)c1ccc3c(c1)OCCO3)C2] | Example 94-12 |
| 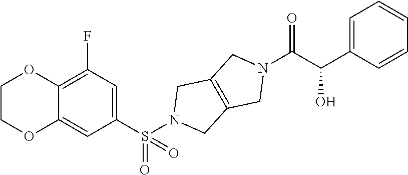 [SMILES: O=C([C@@H](O)c1ccccc1)N1CC2=C(C1)CN(S(=O)(=O)c1cc(F)c3c(c1)OCCO3)C2] | Example 94-13 |
| 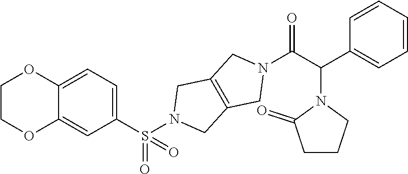 [SMILES: O=C(C(c1ccccc1)N1CCCC1=O)N1CC2=C(C1)CN(S(=O)(=O)c1ccc3c(c1)OCCO3)C2] | Examples 94-14 and 94-15 |
| 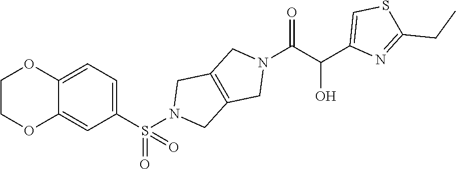 [SMILES: CCc1nc(C(O)C(=O)N2CC3=C(C2)CN(S(=O)(=O)c2ccc4c(c2)OCCO4)C3)cs1] | Example 94-16 |
| 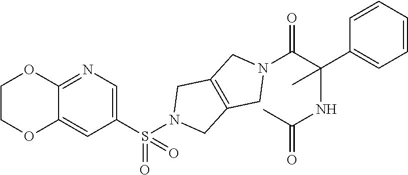 [SMILES: CC(=O)NC(C)(C(=O)N1CC2=C(C1)CN(S(=O)(=O)c1cnc3c(c1)OCCO3)C2)c1ccccc1] | Examples 94-17 and 94-18 |
| 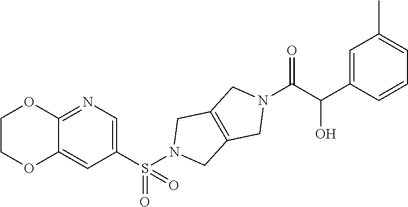 [SMILES: Cc1cccc(C(O)C(=O)N2CC3=C(C2)CN(S(=O)(=O)c2cnc4c(c2)OCCO4)C3)c1] | Examples 94-19 and 94-20 |
| 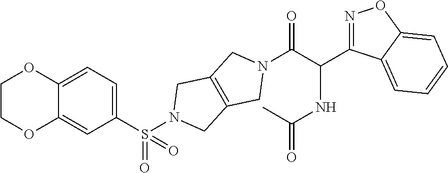 [SMILES: CC(=O)NC(C(=O)N1CC2=C(C1)CN(S(=O)(=O)c1ccc3c(c1)OCCO3)C2)c1noc2ccccc12] | Examples 94-21 and 94-22 |
| 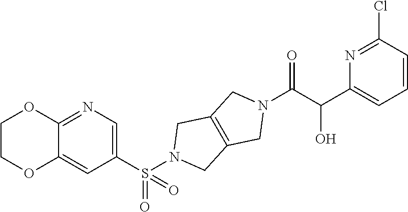 [SMILES: O=C(C(O)c1cccc(Cl)n1)N1CC2=C(C1)CN(S(=O)(=O)c1cnc3c(c1)OCCO3)C2] | Examples 94-23 and 94-24 |
| 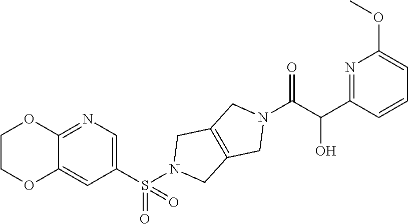 [SMILES: COc1cccc(C(O)C(=O)N2CC3=C(C2)CN(S(=O)(=O)c2cnc4c(c2)OCCO4)C3)n1] | Examples 94-25 and 94-26 |
| 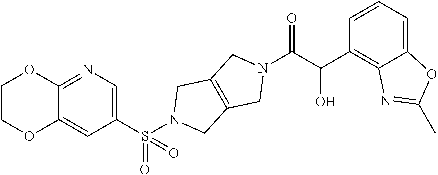 [SMILES: Cc1nc2c(C(O)C(=O)N3CC4=C(C3)CN(S(=O)(=O)c3cnc5c(c3)OCCO5)C4)cccc2o1] | Examples 94-27 and 94-28 |
| 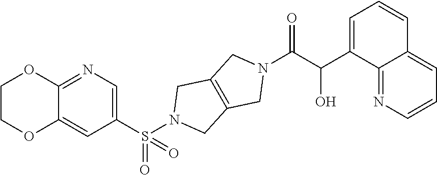 [SMILES: O=C(C(O)c1cccc2cccnc12)N1CC2=C(C1)CN(S(=O)(=O)c1cnc3c(c1)OCCO3)C2] | Examples 94-29 and 94-30 |
| 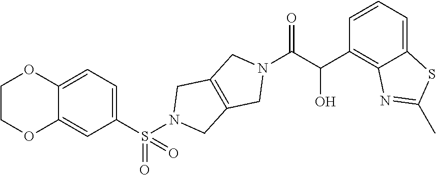 [SMILES: Cc1nc2c(C(O)C(=O)N3CC4=C(C3)CN(S(=O)(=O)c3ccc5c(c3)OCCO5)C4)cccc2s1] | Examples 94-31 and 94-32 |
| 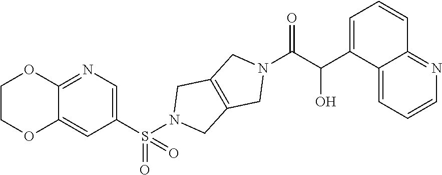 [SMILES: O=C(C(O)c1cccc2ncccc12)N1CC2=C(C1)CN(S(=O)(=O)c1cnc3c(c1)OCCO3)C2] | Examples 94-33 and 94-34 |
| 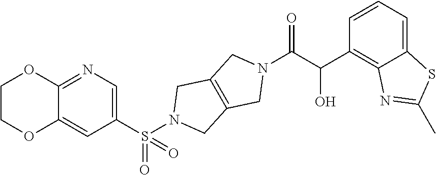 [SMILES: Cc1nc2c(C(O)C(=O)N3CC4=C(C3)CN(S(=O)(=O)c3cnc5c(c3)OCCO5)C4)cccc2s1] | Examples 94-35 and 94-36 |
| 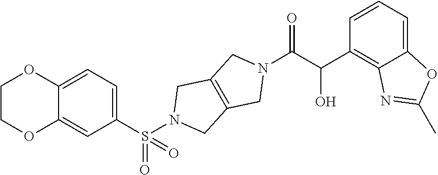 [SMILES: Cc1nc2c(C(O)C(=O)N3CC4=C(C3)CN(S(=O)(=O)c3ccc5c(c3)OCCO5)C4)cccc2o1] | Examples 94-37 and 94-38 |
| 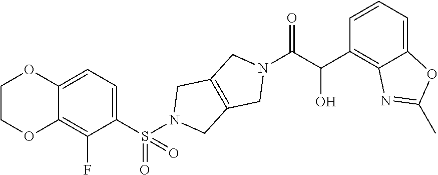 [SMILES: Cc1nc2c(C(O)C(=O)N3CC4=C(C3)CN(S(=O)(=O)c3ccc5c(c3F)OCCO5)C4)cccc2o1] | Examples 94-39 and 94-40 |
| 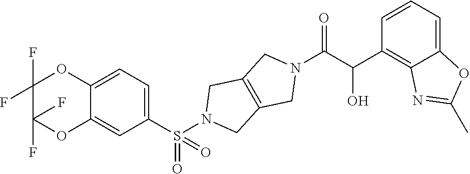 | Examples 94-41 and 94-42 |
| 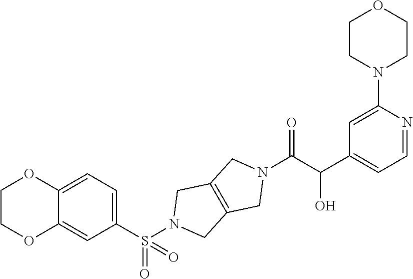 [SMILES: O=C(C(O)c1ccnc(N2CCOCC2)c1)N1CC2=C(C1)CN(S(=O)(=O)c1ccc3c(c1)OCCO3)C2] | Examples 94-43 and 94-44 |
| 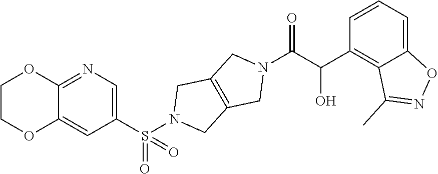 [SMILES: Cc1noc2cccc(C(O)C(=O)N3CC4=C(C3)CN(S(=O)(=O)c3cnc5c(c3)OCCO5)C4)c12] | Examples 94-45 and 94-46 |
| 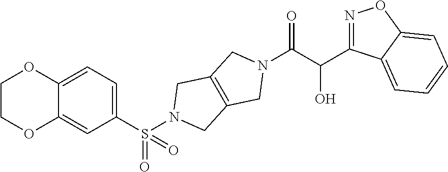 [SMILES: O=C(C(O)c1noc2ccccc12)N1CC2=C(C1)CN(S(=O)(=O)c1ccc3c(c1)OCCO3)C2] | Examples 94-47 and 94-48 |
| 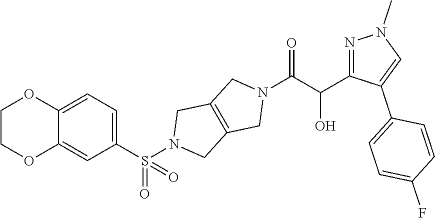 [SMILES: Cn1cc(-c2ccc(F)cc2)c(C(O)C(=O)N2CC3=C(C2)CN(S(=O)(=O)c2ccc4c(c2)OCCO4)C3)n1] | Examples 94-49 and 94-50 |
| 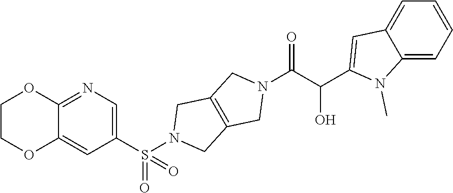 [SMILES: Cn1c(C(O)C(=O)N2CC3=C(C2)CN(S(=O)(=O)c2cnc4c(c2)OCCO4)C3)cc2ccccc21] | Examples 94-51 and 94-52 |
| 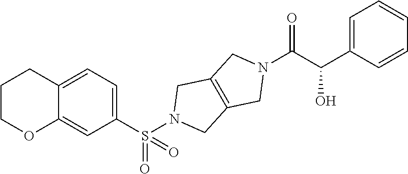 [SMILES: O=C([C@@H](O)c1ccccc1)N1CC2=C(C1)CN(S(=O)(=O)c1ccc3c(c1)OCCC3)C2] | Example 94-53 |
| 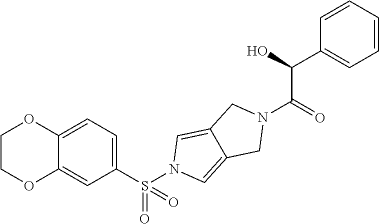 [SMILES: O=C([C@@H](O)c1ccccc1)N1Cc2cn(S(=O)(=O)c3ccc4c(c3)OCCO4)cc2C1] | Example 94-54 |
| 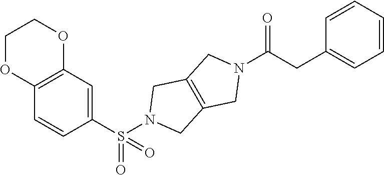 [SMILES: O=C(Cc1ccccc1)N1CC2=C(C1)CN(S(=O)(=O)c1ccc3c(c1)OCCO3)C2] | Example 94-55 |
| 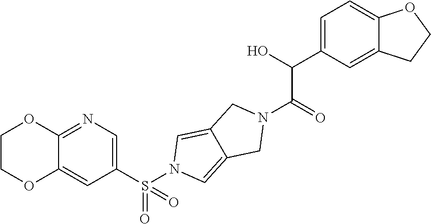 [SMILES: O=C(C(O)c1ccc2c(c1)CCO2)N1Cc2cn(S(=O)(=O)c3cnc4c(c3)OCCO4)cc2C1] | Example 94-56 |
| 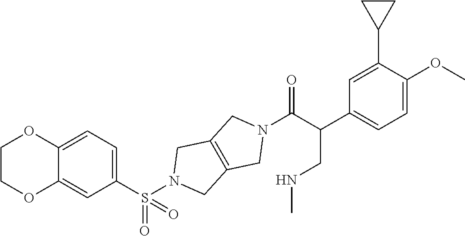 [SMILES: CNCC(C(=O)N1CC2=C(C1)CN(S(=O)(=O)c1ccc3c(c1)OCCO3)C2)c1ccc(OC)c(C2CC2)c1] | Examples 95-1 and 95-2 |
| 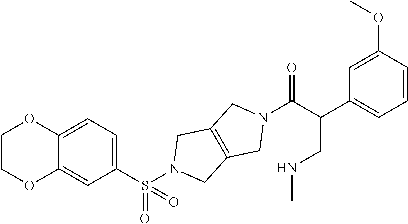 [SMILES: CNCC(C(=O)N1CC2=C(C1)CN(S(=O)(=O)c1ccc3c(c1)OCCO3)C2)c1cccc(OC)c1] | Examples 95-3 and 95-4 |
| 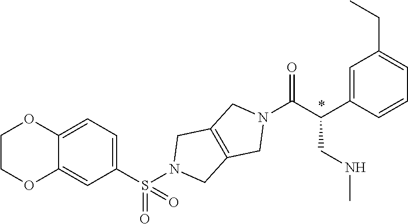 [SMILES: CCc1cccc([C@H](CNC)C(=O)N2CC3=C(C2)CN(S(=O)(=O)c2ccc4c(c2)OCCO4)C3)c1] | Example 95-5 |
| 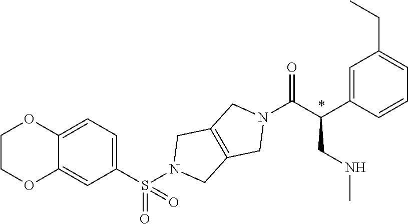 [SMILES: CCc1cccc([C@@H](CNC)C(=O)N2CC3=C(C2)CN(S(=O)(=O)c2ccc4c(c2)OCCO4)C3)c1] | Example 95-6 |
| 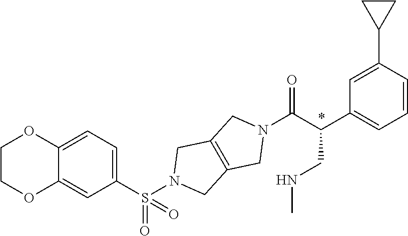 [SMILES: CNC[C@H](C(=O)N1CC2=C(C1)CN(S(=O)(=O)c1ccc3c(c1)OCCO3)C2)c1cccc(C2CC2)c1] | Example 95-7 |
| 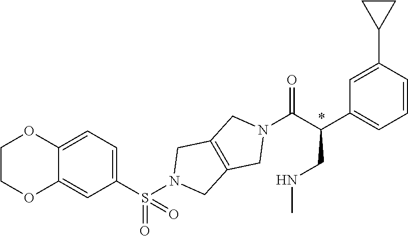 [SMILES: CNC[C@@H](C(=O)N1CC2=C(C1)CN(S(=O)(=O)c1ccc3c(c1)OCCO3)C2)c1cccc(C2CC2)c1] | Example 95-8 |
| 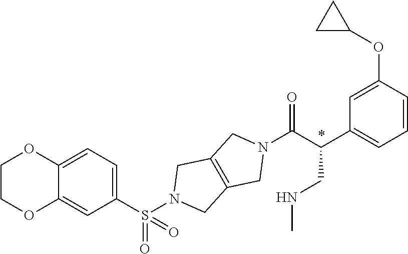 [SMILES: CNC[C@H](C(=O)N1CC2=C(C1)CN(S(=O)(=O)c1ccc3c(c1)OCCO3)C2)c1cccc(OC2CC2)c1] | Example 95-9 |
| 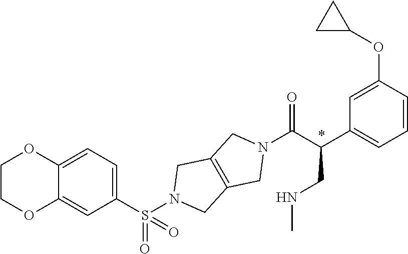 [SMILES: CNC[C@@H](C(=O)N1CC2=C(C1)CN(S(=O)(=O)c1ccc3c(c1)OCCO3)C2)c1cccc(OC2CC2)c1] | Example 95-10 |
| 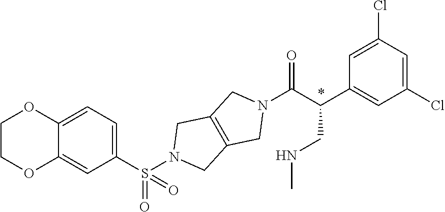 [SMILES: CNC[C@H](C(=O)N1CC2=C(C1)CN(S(=O)(=O)c1ccc3c(c1)OCCO3)C2)c1cc(Cl)cc(Cl)c1] | Example 95-11 |
| 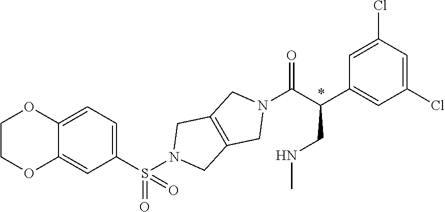 [SMILES: CNC[C@@H](C(=O)N1CC2=C(C1)CN(S(=O)(=O)c1ccc3c(c1)OCCO3)C2)c1cc(Cl)cc(Cl)c1] | Example 95-12 |
| 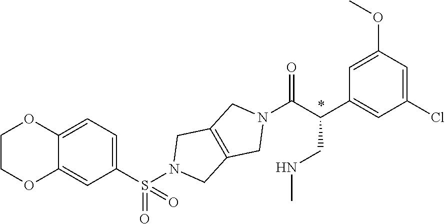 [SMILES: CNC[C@H](C(=O)N1CC2=C(C1)CN(S(=O)(=O)c1ccc3c(c1)OCCO3)C2)c1cc(Cl)cc(OC)c1] | Example 95-13 |
| 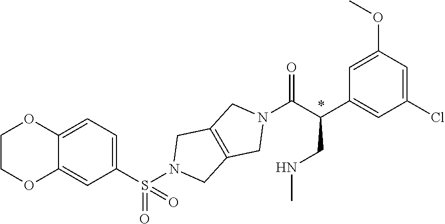 [SMILES: CNC[C@@H](C(=O)N1CC2=C(C1)CN(S(=O)(=O)c1ccc3c(c1)OCCO3)C2)c1cc(Cl)cc(OC)c1] | Example 95-14 |
| 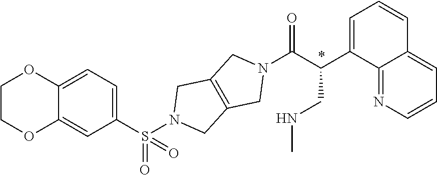 [SMILES: CNC[C@H](C(=O)N1CC2=C(C1)CN(S(=O)(=O)c1ccc3c(c1)OCCO3)C2)c1cccc2cccnc12] | Example 95-15 |
| 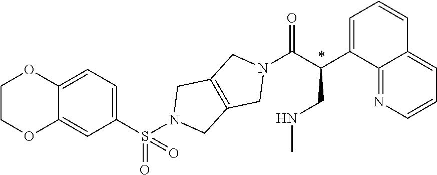 [SMILES: CNC[C@@H](C(=O)N1CC2=C(C1)CN(S(=O)(=O)c1ccc3c(c1)OCCO3)C2)c1cccc2cccnc12] | Example 95-16 |
| 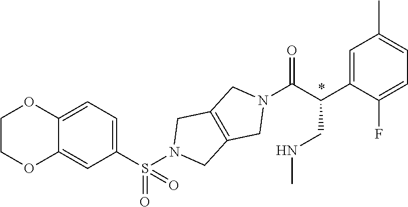 [SMILES: CNC[C@H](C(=O)N1CC2=C(C1)CN(S(=O)(=O)c1ccc3c(c1)OCCO3)C2)c1cc(C)ccc1F] | Example 95-17 |
| 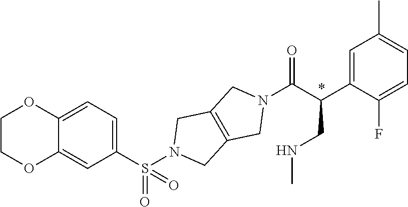 [SMILES: CNC[C@@H](C(=O)N1CC2=C(C1)CN(S(=O)(=O)c1ccc3c(c1)OCCO3)C2)c1cc(C)ccc1F] | Example 95-18 |
| 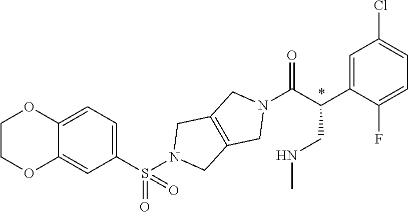 [SMILES: CNC[C@H](C(=O)N1CC2=C(C1)CN(S(=O)(=O)c1ccc3c(c1)OCCO3)C2)c1cc(Cl)ccc1F] | Example 95-19 |
| 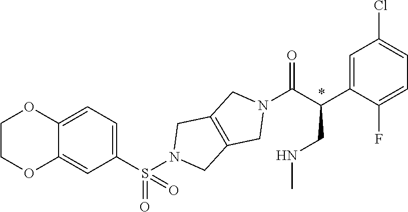 [SMILES: CNC[C@@H](C(=O)N1CC2=C(C1)CN(S(=O)(=O)c1ccc3c(c1)OCCO3)C2)c1cc(Cl)ccc1F] | Example 95-20 |
| 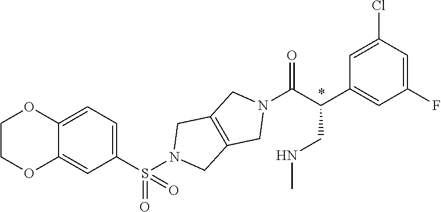 [SMILES: CNC[C@H](C(=O)N1CC2=C(C1)CN(S(=O)(=O)c1ccc3c(c1)OCCO3)C2)c1cc(F)cc(Cl)c1] | Example 95-21 |
| 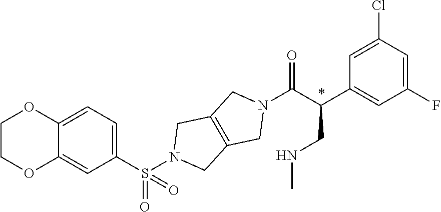 [SMILES: CNC[C@@H](C(=O)N1CC2=C(C1)CN(S(=O)(=O)c1ccc3c(c1)OCCO3)C2)c1cc(F)cc(Cl)c1] | Example 95-22 |
| 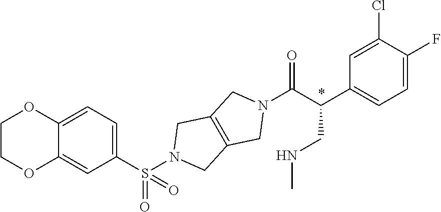 [SMILES: CNC[C@H](C(=O)N1CC2=C(C1)CN(S(=O)(=O)c1ccc3c(c1)OCCO3)C2)c1ccc(F)c(Cl)c1] | Example 95-23 |
| 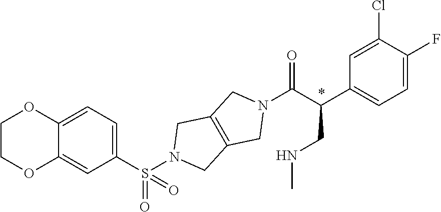 [SMILES: CNC[C@@H](C(=O)N1CC2=C(C1)CN(S(=O)(=O)c1ccc3c(c1)OCCO3)C2)c1ccc(F)c(Cl)c1] | Example 95-24 |
| 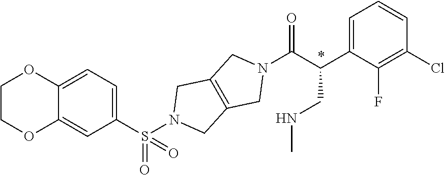 [SMILES: CNC[C@H](C(=O)N1CC2=C(C1)CN(S(=O)(=O)c1ccc3c(c1)OCCO3)C2)c1cccc(Cl)c1F] | Example 95-25 |
| 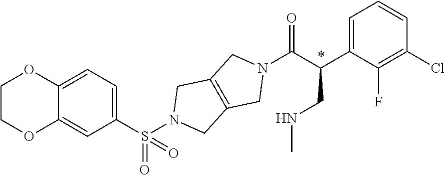 [SMILES: CNC[C@@H](C(=O)N1CC2=C(C1)CN(S(=O)(=O)c1ccc3c(c1)OCCO3)C2)c1cccc(Cl)c1F] | Example 95-26 |
| 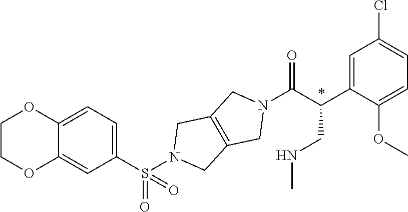 [SMILES: CNC[C@H](C(=O)N1CC2=C(C1)CN(S(=O)(=O)c1ccc3c(c1)OCCO3)C2)c1cc(Cl)ccc1OC] | Example 95-27 |
| 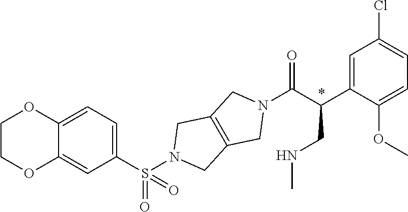 [SMILES: CNC[C@@H](C(=O)N1CC2=C(C1)CN(S(=O)(=O)c1ccc3c(c1)OCCO3)C2)c1cc(Cl)ccc1OC] | Example 95-28 |
| 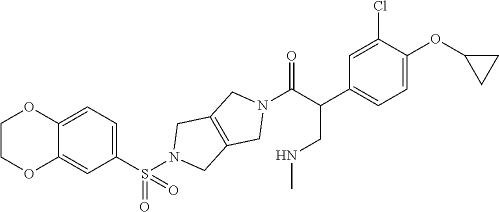 [SMILES: CNCC(C(=O)N1CC2=C(C1)CN(S(=O)(=O)c1ccc3c(c1)OCCO3)C2)c1ccc(OC2CC2)c(Cl)c1] | Examples 95-29 and 95-30 |
| 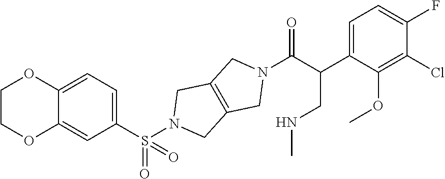 [SMILES: CNCC(C(=O)N1CC2=C(C1)CN(S(=O)(=O)c1ccc3c(c1)OCCO3)C2)c1ccc(F)c(Cl)c1OC] | Examples 95-31 and 95-32 |
| 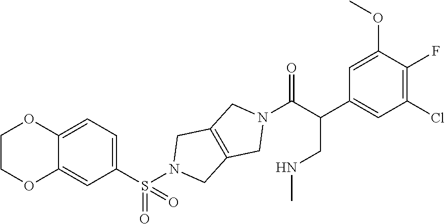 [SMILES: CNCC(C(=O)N1CC2=C(C1)CN(S(=O)(=O)c1ccc3c(c1)OCCO3)C2)c1cc(Cl)c(F)c(OC)c1] | Examples 95-33 and 95-34 |
| 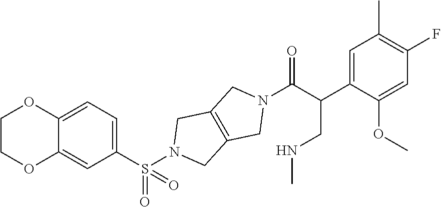 [SMILES: CNCC(C(=O)N1CC2=C(C1)CN(S(=O)(=O)c1ccc3c(c1)OCCO3)C2)c1cc(C)c(F)cc1OC] | Examples 95-35 and 95-36 |
| 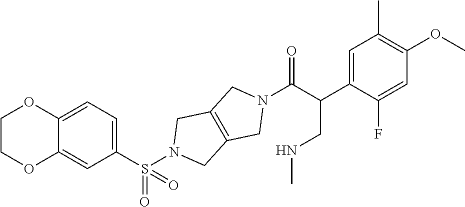 [SMILES: CNCC(C(=O)N1CC2=C(C1)CN(S(=O)(=O)c1ccc3c(c1)OCCO3)C2)c1cc(C)c(OC)cc1F] | Examples 95-37 and 95-38 |
| 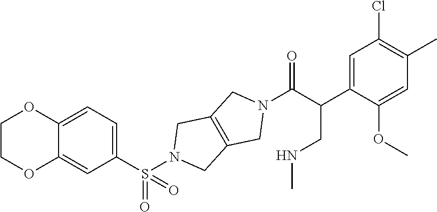 [SMILES: CNCC(C(=O)N1CC2=C(C1)CN(S(=O)(=O)c1ccc3c(c1)OCCO3)C2)c1cc(Cl)c(C)cc1OC] | Examples 95-39 and 95-40 |
| 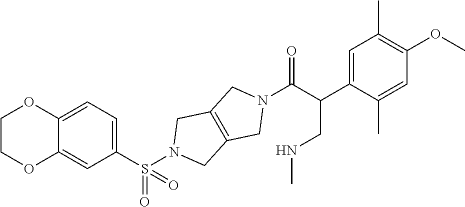 [SMILES: CNCC(C(=O)N1CC2=C(C1)CN(S(=O)(=O)c1ccc3c(c1)OCCO3)C2)c1cc(C)c(OC)cc1C] | Examples 95-41 and 95-42 |
| 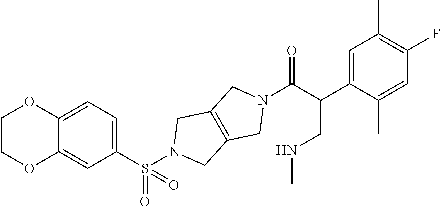 [SMILES: CNCC(C(=O)N1CC2=C(C1)CN(S(=O)(=O)c1ccc3c(c1)OCCO3)C2)c1cc(C)c(F)cc1C] | Examples 95-43 and 95-44 |
| 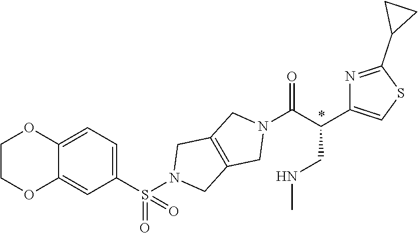 [SMILES: CNC[C@H](C(=O)N1CC2=C(C1)CN(S(=O)(=O)c1ccc3c(c1)OCCO3)C2)c1csc(C2CC2)n1] | Example 95-45 |
| 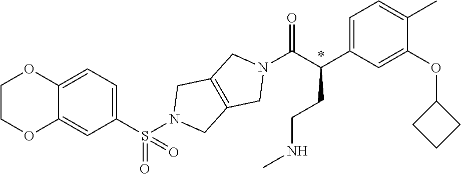 [SMILES: CNCC[C@@H](C(=O)N1CC2=C(C1)CN(S(=O)(=O)c1ccc3c(c1)OCCO3)C2)c1ccc(C)c(OC2CCC2)c1] | Example 95-46 |
| 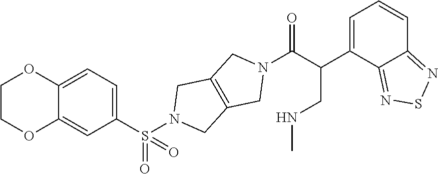 [SMILES: CNCC(C(=O)N1CC2=C(C1)CN(S(=O)(=O)c1ccc3c(c1)OCCO3)C2)c1cccc2nsnc12] | Examples 95-47 and 95-48 |
| 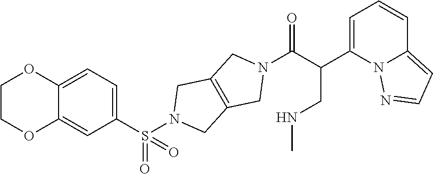 [SMILES: CNCC(C(=O)N1CC2=C(C1)CN(S(=O)(=O)c1ccc3c(c1)OCCO3)C2)c1cccc2ccnn12] | Examples 95-49 and 95-50 |
| 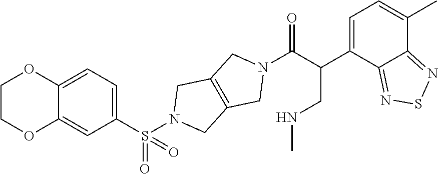 [SMILES: CNCC(C(=O)N1CC2=C(C1)CN(S(=O)(=O)c1ccc3c(c1)OCCO3)C2)c1ccc(C)c2nsnc12] | Examples 95-51 and 95-52 |
| 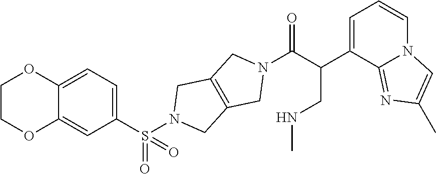 [SMILES: CNCC(C(=O)N1CC2=C(C1)CN(S(=O)(=O)c1ccc3c(c1)OCCO3)C2)c1cccn2cc(C)nc12] | Examples 95-53 and 95-54 |
| 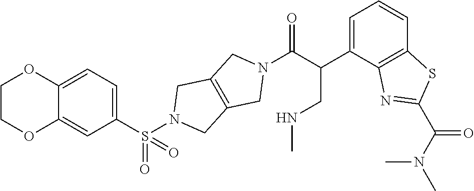 [SMILES: CNCC(C(=O)N1CC2=C(C1)CN(S(=O)(=O)c1ccc3c(c1)OCCO3)C2)c1cccc2sc(C(=O)N(C)C)nc12] | Examples 95-55 and 95-56 |
| 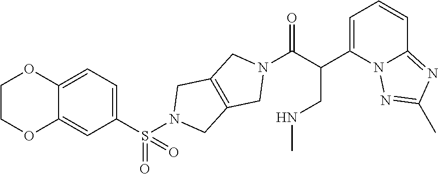 [SMILES: CNCC(C(=O)N1CC2=C(C1)CN(S(=O)(=O)c1ccc3c(c1)OCCO3)C2)c1cccc2nc(C)nn12] | Example 95-57 |
| 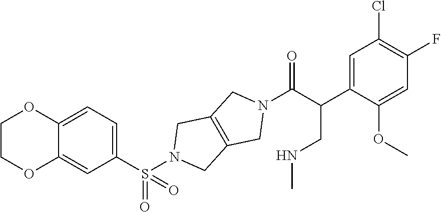 [SMILES: CNCC(C(=O)N1CC2=C(C1)CN(S(=O)(=O)c1ccc3c(c1)OCCO3)C2)c1cc(Cl)c(F)cc1OC] | Examples 95-58 and 95-59 |
| 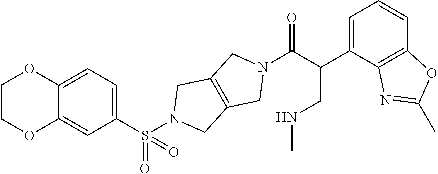 [SMILES: CNCC(C(=O)N1CC2=C(C1)CN(S(=O)(=O)c1ccc3c(c1)OCCO3)C2)c1cccc2oc(C)nc12] | Examples 95-60 and 95-61 |
| 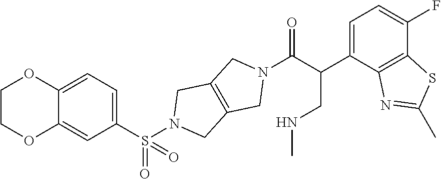 [SMILES: CNCC(C(=O)N1CC2=C(C1)CN(S(=O)(=O)c1ccc3c(c1)OCCO3)C2)c1ccc(F)c2sc(C)nc12] | Examples 95-62 and 95-63 |
| 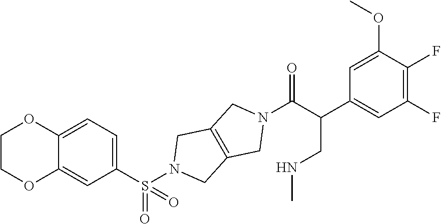 [SMILES: CNCC(C(=O)N1CC2=C(C1)CN(S(=O)(=O)c1ccc3c(c1)OCCO3)C2)c1cc(F)c(F)c(OC)c1] | Examples 95-64 and 95-65 |
| 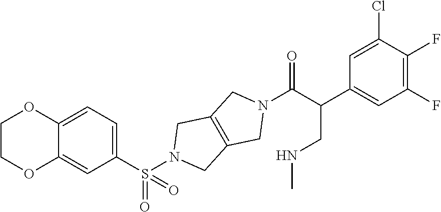 [SMILES: CNCC(C(=O)N1CC2=C(C1)CN(S(=O)(=O)c1ccc3c(c1)OCCO3)C2)c1cc(F)c(F)c(Cl)c1] | Examples 95-66 and 95-67 |
| 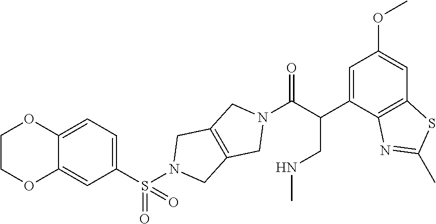 [SMILES: CNCC(C(=O)N1CC2=C(C1)CN(S(=O)(=O)c1ccc3c(c1)OCCO3)C2)c1cc(OC)cc2sc(C)nc12] | Examples 95-68 and 95-69 |
| 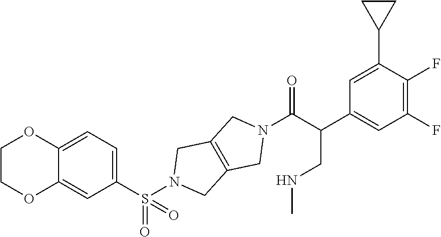 [SMILES: CNCC(C(=O)N1CC2=C(C1)CN(S(=O)(=O)c1ccc3c(c1)OCCO3)C2)c1cc(F)c(F)c(C2CC2)c1] | Examples 95-70 and 95-71 |
| 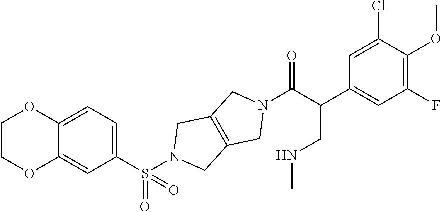 [SMILES: CNCC(C(=O)N1CC2=C(C1)CN(S(=O)(=O)c1ccc3c(c1)OCCO3)C2)c1cc(F)c(OC)c(Cl)c1] | Examples 95-72 and 95-73 |
| 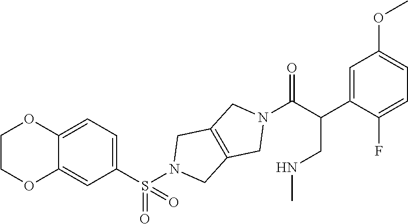 [SMILES: CNCC(C(=O)N1CC2=C(C1)CN(S(=O)(=O)c1ccc3c(c1)OCCO3)C2)c1cc(OC)ccc1F] | Examples 95-74 and 95-75 |
| 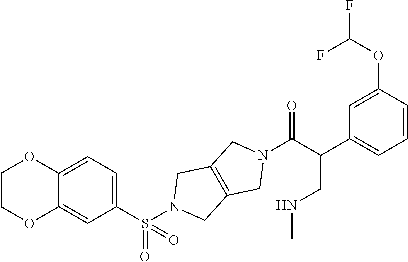 [SMILES: CNCC(C(=O)N1CC2=C(C1)CN(S(=O)(=O)c1ccc3c(c1)OCCO3)C2)c1cccc(OC(F)F)c1] | Examples 95-76 and 95-77 |
| 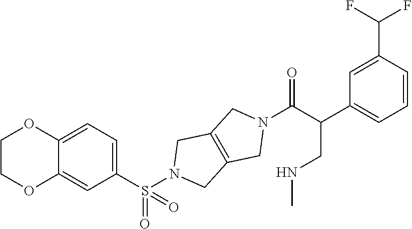 [SMILES: CNCC(C(=O)N1CC2=C(C1)CN(S(=O)(=O)c1ccc3c(c1)OCCO3)C2)c1cccc(C(F)F)c1] | Examples 95-78 and 95-79 |
| 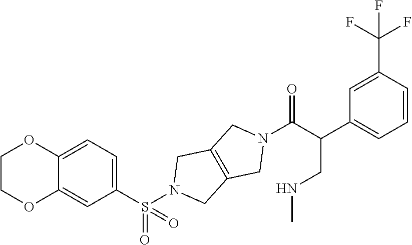 [SMILES: CNCC(C(=O)N1CC2=C(C1)CN(S(=O)(=O)c1ccc3c(c1)OCCO3)C2)c1cccc(C(F)(F)F)c1] | Examples 95-80 and 95-81 |
| 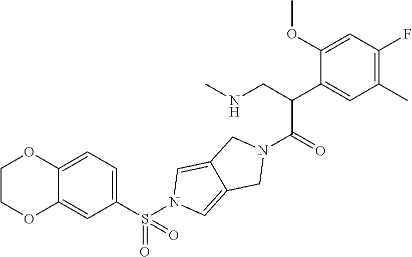 [SMILES: CNCC(C(=O)N1Cc2cn(S(=O)(=O)c3ccc4c(c3)OCCO4)cc2C1)c1cc(C)c(F)cc1OC] | Examples 95-82 and 95-83 |
| 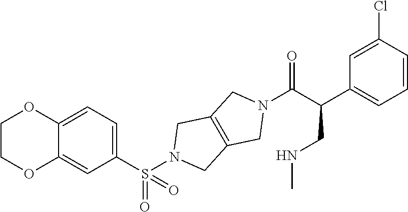 [SMILES: CNC[C@@H](C(=O)N1CC2=C(C1)CN(S(=O)(=O)c1ccc3c(c1)OCCO3)C2)c1cccc(Cl)c1] | Example 95-84 |
| 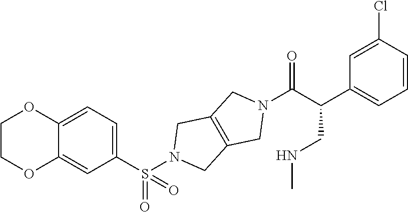 [SMILES: CNC[C@H](C(=O)N1CC2=C(C1)CN(S(=O)(=O)c1ccc3c(c1)OCCO3)C2)c1cccc(Cl)c1] | Example 95-85, 99-1 |
| 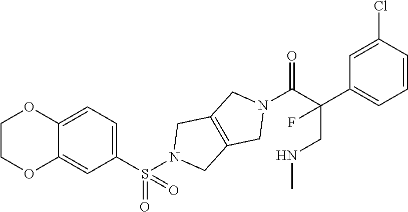 [SMILES: CNCC(F)(C(=O)N1CC2=C(C1)CN(S(=O)(=O)c1ccc3c(c1)OCCO3)C2)c1cccc(Cl)c1] | Examples 96-1 and 96-2 |
| 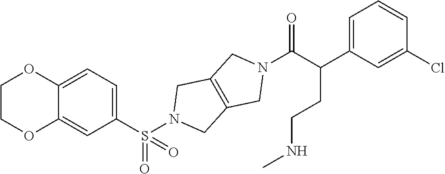 [SMILES: CNCCC(C(=O)N1CC2=C(C1)CN(S(=O)(=O)c1ccc3c(c1)OCCO3)C2)c1cccc(Cl)c1] | Examples 96-3 and 96-4 |
| 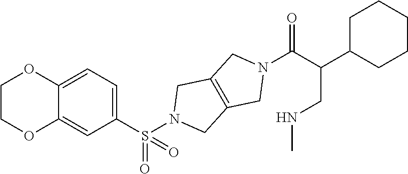 [SMILES: CNCC(C(=O)N1CC2=C(C1)CN(S(=O)(=O)c1ccc3c(c1)OCCO3)C2)C1CCCCC1] | Examples 96-5 and 96-6 |
| 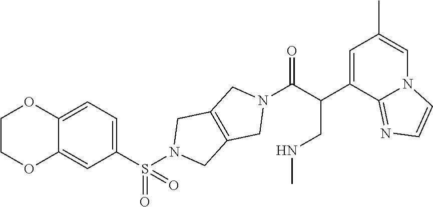 [SMILES: CNCC(C(=O)N1CC2=C(C1)CN(S(=O)(=O)c1ccc3c(c1)OCCO3)C2)c1cc(C)cn2ccnc12] | Example 96-7 |
| 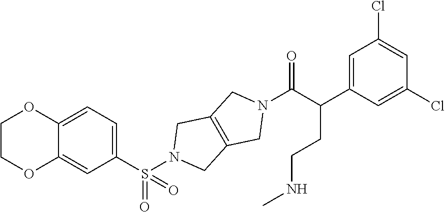 [SMILES: CNCCC(C(=O)N1CC2=C(C1)CN(S(=O)(=O)c1ccc3c(c1)OCCO3)C2)c1cc(Cl)cc(Cl)c1] | Examples 96-8 and 96-9 |
| 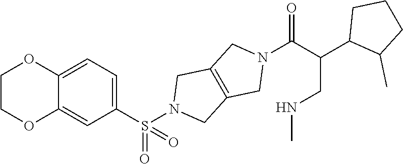 [SMILES: CNCC(C(=O)N1CC2=C(C1)CN(S(=O)(=O)c1ccc3c(c1)OCCO3)C2)C1CCCC1C] | Examples 96-10, 96-11, 96-12, 96-13, 96-14, 96-15, 96-16, and 96-17 |
| 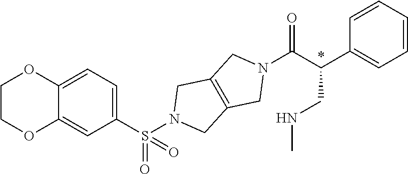 [SMILES: CNC[C@H](C(=O)N1CC2=C(C1)CN(S(=O)(=O)c1ccc3c(c1)OCCO3)C2)c1ccccc1] | Example 96-18 |
| 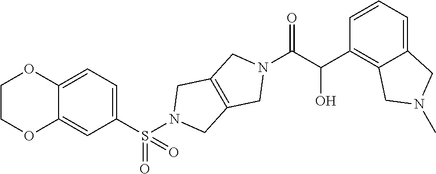 [SMILES: CN1Cc2cccc(C(O)C(=O)N3CC4=C(C3)CN(S(=O)(=O)c3ccc5c(c3)OCCO5)C4)c2C1] | Examples 97-1 and 97-2 |
| 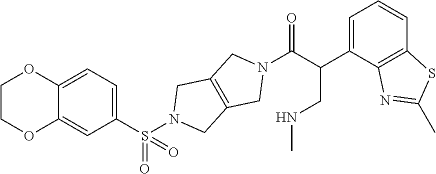 [SMILES: CNCC(C(=O)N1CC2=C(C1)CN(S(=O)(=O)c1ccc3c(c1)OCCO3)C2)c1cccc2sc(C)nc12] | Examples 97-3 and 97-4 |
| 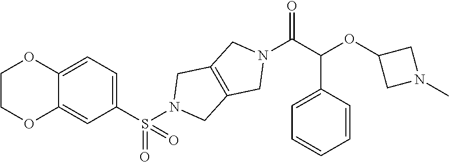 [SMILES: CN1CC(OC(C(=O)N2CC3=C(C2)CN(S(=O)(=O)c2ccc4c(c2)OCCO4)C3)c2ccccc2)C1] | Examples 97-5 and 97-6 |
| 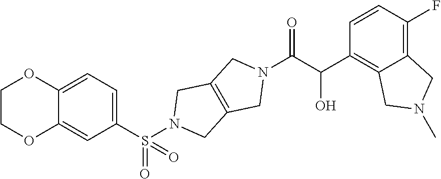 [SMILES: CN1Cc2c(F)ccc(C(O)C(=O)N3CC4=C(C3)CN(S(=O)(=O)c3ccc5c(c3)OCCO5)C4)c2C1] | Examples 97-7 and 97-8 |
| 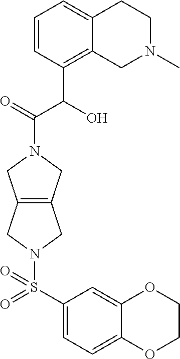 [SMILES: CN1CCc2cccc(C(O)C(=O)N3CC4=C(C3)CN(S(=O)(=O)c3ccc5c(c3)OCCO5)C4)c2C1] | Examples 97-9 and 97-10 |
| 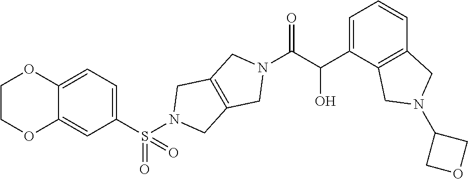 [SMILES: O=C(C(O)c1cccc2c1CN(C1COC1)C2)N1CC2=C(C1)CN(S(=O)(=O)c1ccc3c(c1)OCCO3)C2] | Examples 97-11 and 97-12 |
| 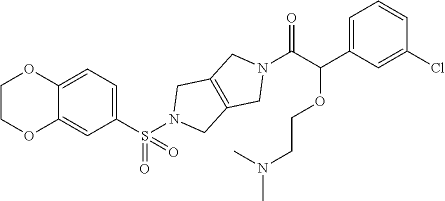 [SMILES: CN(C)CCOC(C(=O)N1CC2=C(C1)CN(S(=O)(=O)c1ccc3c(c1)OCCO3)C2)c1cccc(Cl)c1] | Examples 97-13 and 97-14 |
| 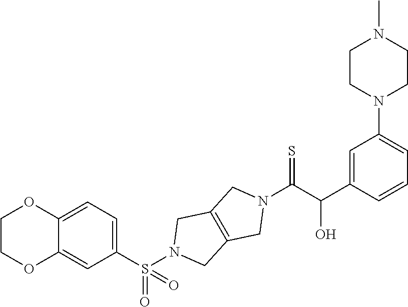 [SMILES: CN1CCN(c2cccc(C(O)C(=S)N3CC4=C(C3)CN(S(=O)(=O)c3ccc5c(c3)OCCO5)C4)c2)CC1] | Examples 97-15 and 97-16 |
| 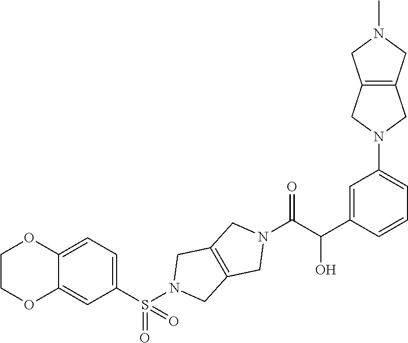 [SMILES: CN1CC2=C(C1)CN(c1cccc(C(O)C(=O)N3CC4=C(C3)CN(S(=O)(=O)c3ccc5c(c3)OCCO5)C4)c1)C2] | Examples 97-17 and 97-18 |
| 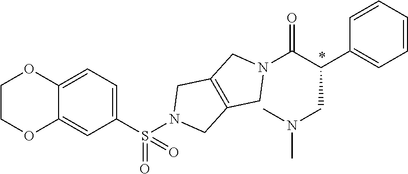 [SMILES: CN(C)C[C@H](C(=O)N1CC2=C(C1)CN(S(=O)(=O)c1ccc3c(c1)OCCO3)C2)c1ccccc1] | Example 97-19 |
| 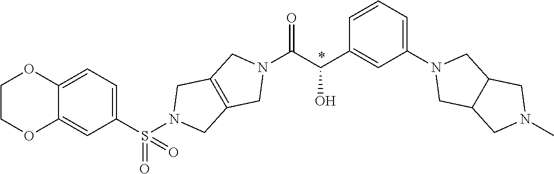 [SMILES: CN1CC2CN(c3cccc([C@H](O)C(=O)N4CC5=C(C4)CN(S(=O)(=O)c4ccc6c(c4)OCCO6)C5)c3)CC2C1] | Example 97-20 |
| 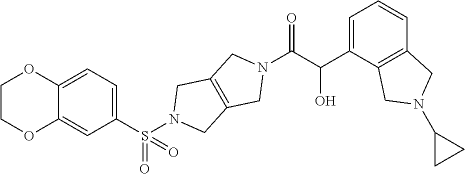 [SMILES: O=C(C(O)c1cccc2c1CN(C1CC1)C2)N1CC2=C(C1)CN(S(=O)(=O)c1ccc3c(c1)OCCO3)C2] | Examples 98-1 and 98-2 |
| 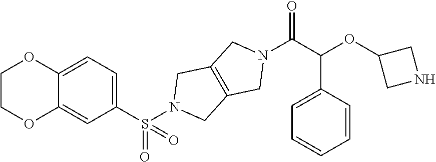 [SMILES: O=C(C(OC1CNC1)c1ccccc1)N1CC2=C(C1)CN(S(=O)(=O)c1ccc3c(c1)OCCO3)C2] | Examples 99-2 and 99-3 |
| 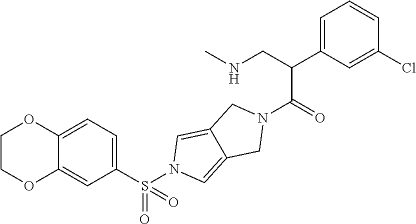 [SMILES: CNCC(C(=O)N1Cc2cn(S(=O)(=O)c3ccc4c(c3)OCCO4)cc2C1)c1cccc(Cl)c1] | Examples 99-4 and 99-5 |
| 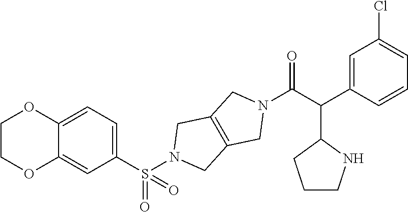 [SMILES: O=C(C(c1cccc(Cl)c1)C1CCCN1)N1CC2=C(C1)CN(S(=O)(=O)c1ccc3c(c1)OCCO3)C2] | Examples 99-6 and 99-7 |
| 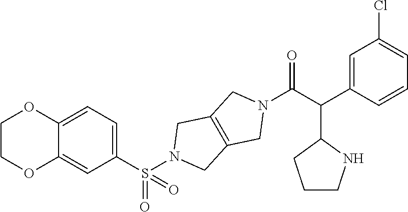 [SMILES: O=C(C(c1cccc(Cl)c1)C1CCCN1)N1CC2=C(C1)CN(S(=O)(=O)c1ccc3c(c1)OCCO3)C2] | Examples 99-8 and 99-9 |
| 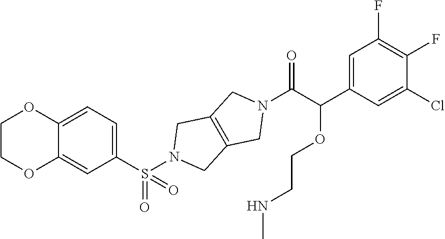 [SMILES: CNCCOC(C(=O)N1CC2=C(C1)CN(S(=O)(=O)c1ccc3c(c1)OCCO3)C2)c1cc(F)c(F)c(Cl)c1] | Examples 99-10 and 99-11 |
| 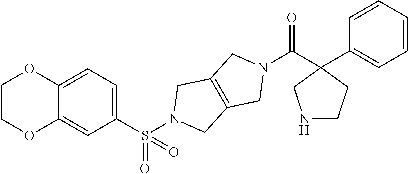 [SMILES: O=C(N1CC2=C(C1)CN(S(=O)(=O)c1ccc3c(c1)OCCO3)C2)C1(c2ccccc2)CCNC1] | Examples 99-12 and 99-13 |
| 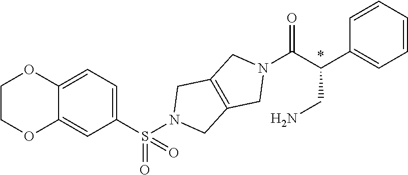 [SMILES: NC[C@H](C(=O)N1CC2=C(C1)CN(S(=O)(=O)c1ccc3c(c1)OCCO3)C2)c1ccccc1] | Example 99-14 |
| 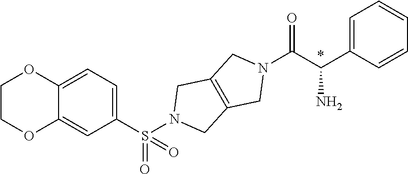 [SMILES: N[C@H](C(=O)N1CC2=C(C1)CN(S(=O)(=O)c1ccc3c(c1)OCCO3)C2)c1ccccc1] | Example 99-15 |
| 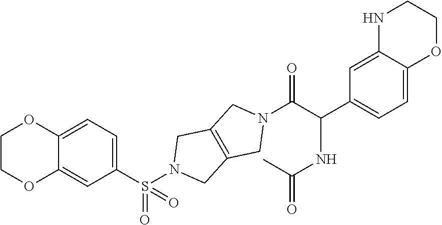 [SMILES: CC(=O)NC(C(=O)N1CC2=C(C1)CN(S(=O)(=O)c1ccc3c(c1)OCCO3)C2)c1ccc2c(c1)NCCO2] | Examples 99-16 and 99-17 |
| 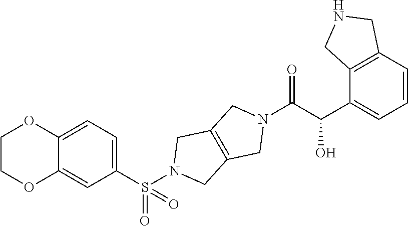 [SMILES: O=C([C@@H](O)c1cccc2c1CNC2)N1CC2=C(C1)CN(S(=O)(=O)c1ccc3c(c1)OCCO3)C2] | Example 99-18 |
| 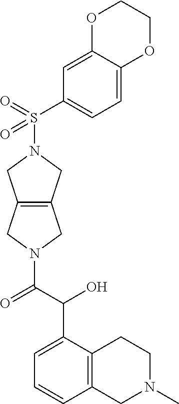 [SMILES: CN1CCc2c(cccc2C(O)C(=O)N2CC3=C(C2)CN(S(=O)(=O)c2ccc4c(c2)OCCO4)C3)C1] | Examples 100-1 and 100-2 |
| 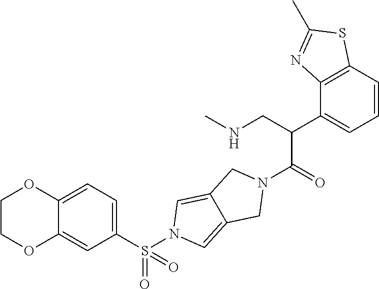 [SMILES: CNCC(C(=O)N1Cc2cn(S(=O)(=O)c3ccc4c(c3)OCCO4)cc2C1)c1cccc2sc(C)nc12] | Examples 100-3 and 100-4 |
| 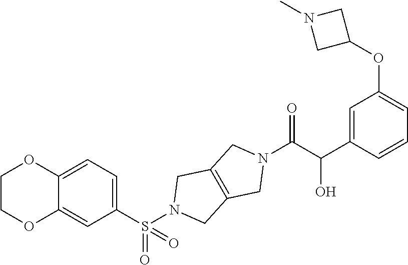 [SMILES: CN1CC(Oc2cccc(C(O)C(=O)N3CC4=C(C3)CN(S(=O)(=O)c3ccc5c(c3)OCCO5)C4)c2)C1] | Examples 100-5 and 100-6 |
| 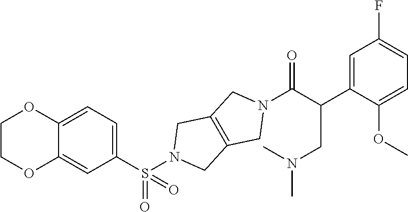 [SMILES: COc1ccc(F)cc1C(CN(C)C)C(=O)N1CC2=C(C1)CN(S(=O)(=O)c1ccc3c(c1)OCCO3)C2] | Examples 101-1 and 101-2 |
| 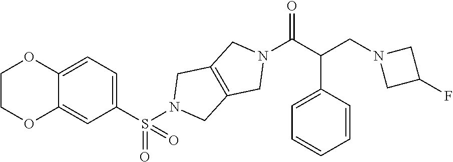 [SMILES: O=C(C(CN1CC(F)C1)c1ccccc1)N1CC2=C(C1)CN(S(=O)(=O)c1ccc3c(c1)OCCO3)C2] | Examples 101-3 and 101-4 |
| 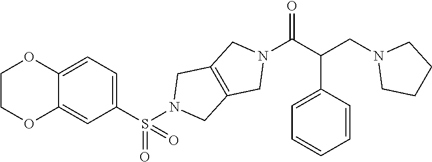 [SMILES: O=C(C(CN1CCCC1)c1ccccc1)N1CC2=C(C1)CN(S(=O)(=O)c1ccc3c(c1)OCCO3)C2] | Example 101-5 |
| 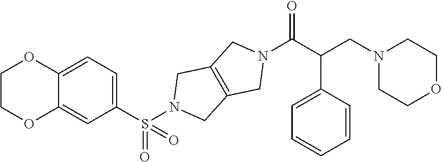 [SMILES: O=C(C(CN1CCOCC1)c1ccccc1)N1CC2=C(C1)CN(S(=O)(=O)c1ccc3c(c1)OCCO3)C2] | Example 101-6 |
| 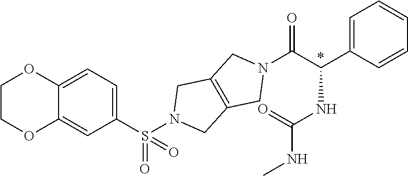 [SMILES: CNC(=O)N[C@H](C(=O)N1CC2=C(C1)CN(S(=O)(=O)c1ccc3c(c1)OCCO3)C2)c1ccccc1] | Example 102-1 |
| 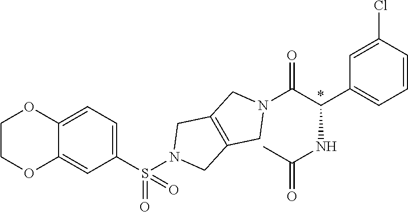 [SMILES: CC(=O)N[C@H](C(=O)N1CC2=C(C1)CN(S(=O)(=O)c1ccc3c(c1)OCCO3)C2)c1cccc(Cl)c1] | Example 102-2 |
| 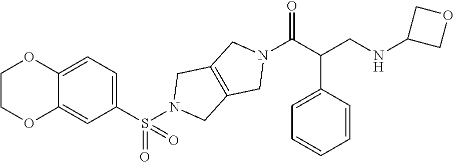 [SMILES: O=C(C(CNC1COC1)c1ccccc1)N1CC2=C(C1)CN(S(=O)(=O)c1ccc3c(c1)OCCO3)C2] | Examples 103-1 and 103-2 |
| 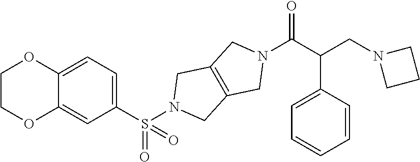 [SMILES: O=C(C(CN1CCC1)c1ccccc1)N1CC2=C(C1)CN(S(=O)(=O)c1ccc3c(c1)OCCO3)C2] | Examples 103-3 and 103-4 |
| 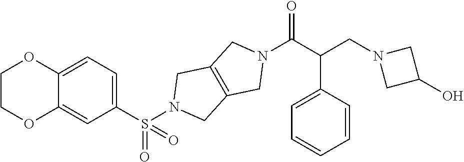 [SMILES: O=C(C(CN1CC(O)C1)c1ccccc1)N1CC2=C(C1)CN(S(=O)(=O)c1ccc3c(c1)OCCO3)C2] | Examples 103-5 and 103-6 |
| 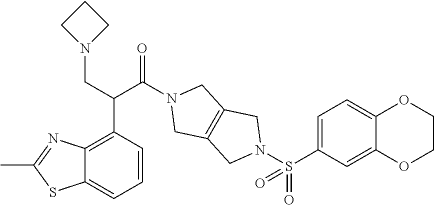 [SMILES: Cc1nc2c(C(CN3CCC3)C(=O)N3CC4=C(C3)CN(S(=O)(=O)c3ccc5c(c3)OCCO5)C4)cccc2s1] | Examples 103-7 and 103-8 |
| 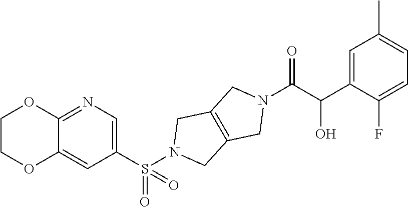 [SMILES: Cc1ccc(F)c(C(O)C(=O)N2CC3=C(C2)CN(S(=O)(=O)c2cnc4c(c2)OCCO4)C3)c1] | Examples 103-9 and 103-10 |
| 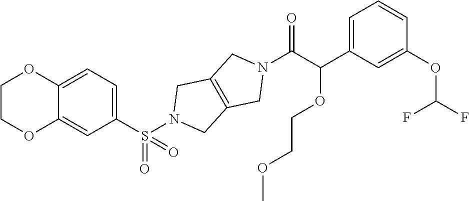 [SMILES: COCCOC(C(=O)N1CC2=C(C1)CN(S(=O)(=O)c1ccc3c(c1)OCCO3)C2)c1cccc(OC(F)F)c1] | Examples 103-11 and 103-12 |
| 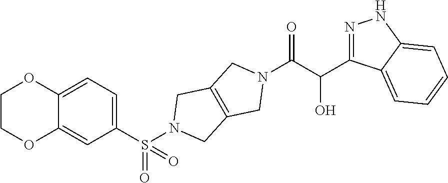 [SMILES: O=C(C(O)c1n[nH]c2ccccc12)N1CC2=C(C1)CN(S(=O)(=O)c1ccc3c(c1)OCCO3)C2] | Examples 103-17 and 103-18 |
| 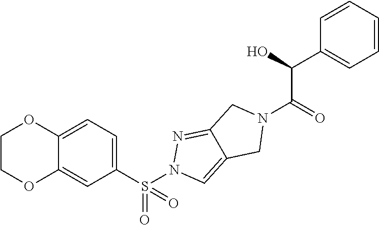 [SMILES: O=C([C@@H](O)c1ccccc1)N1Cc2cn(S(=O)(=O)c3ccc4c(c3)OCCO4)nc2C1] | Example 103-19 |
| 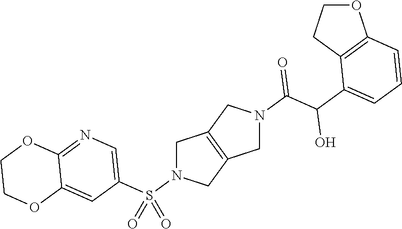 [SMILES: O=C(C(O)c1cccc2c1CCO2)N1CC2=C(C1)CN(S(=O)(=O)c1cnc3c(c1)OCCO3)C2] | Example 103-20 and 103-21 |
| 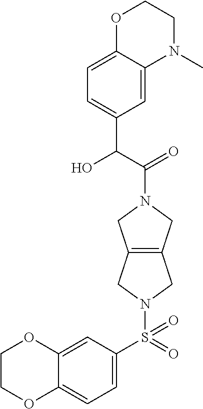 [SMILES: CN1CCOc2ccc(C(O)C(=O)N3CC4=C(C3)CN(S(=O)(=O)c3ccc5c(c3)OCCO5)C4)cc21] | Examples 103-22 and 103-23 |
| 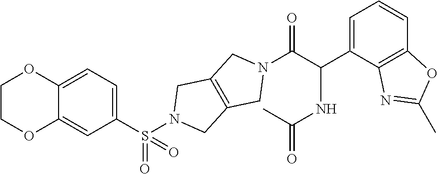 [SMILES: CC(=O)NC(C(=O)N1CC2=C(C1)CN(S(=O)(=O)c1ccc3c(c1)OCCO3)C2)c1cccc2oc(C)nc12] | Examples 103-24 and 103-25 |
| 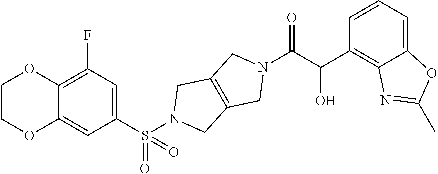 [SMILES: Cc1nc2c(C(O)C(=O)N3CC4=C(C3)CN(S(=O)(=O)c3cc(F)c5c(c3)OCCO5)C4)cccc2o1] | Examples 103-26 and 103-27 |
| 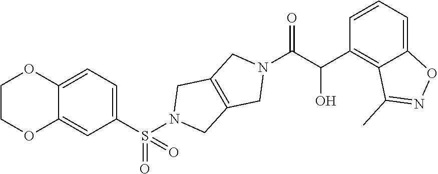 [SMILES: Cc1noc2cccc(C(O)C(=O)N3CC4=C(C3)CN(S(=O)(=O)c3ccc5c(c3)OCCO5)C4)c12] | Examples 103-28 and 103-29 |
| 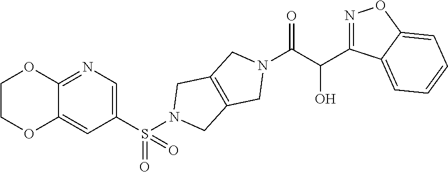 [SMILES: O=C(C(O)c1noc2ccccc12)N1CC2=C(C1)CN(S(=O)(=O)c1cnc3c(c1)OCCO3)C2] | Examples 103-30 and 103-31 |
| 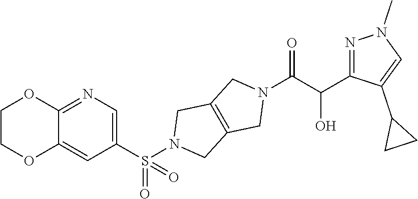 [SMILES: Cn1cc(C2CC2)c(C(O)C(=O)N2CC3=C(C2)CN(S(=O)(=O)c2cnc4c(c2)OCCO4)C3)n1] | Examples 103-32 and 103-33 |
| 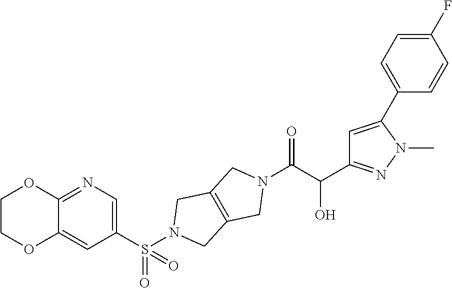 [SMILES: Cn1nc(C(O)C(=O)N2CC3=C(C2)CN(S(=O)(=O)c2cnc4c(c2)OCCO4)C3)cc1-c1ccc(F)cc1] | Examples 103-34 and 103-35 |
| 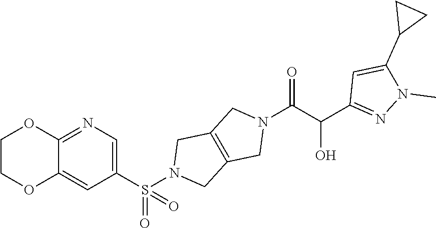 [SMILES: Cn1nc(C(O)C(=O)N2CC3=C(C2)CN(S(=O)(=O)c2cnc4c(c2)OCCO4)C3)cc1C1CC1] | Examples 103-36 and 103-37 |
| 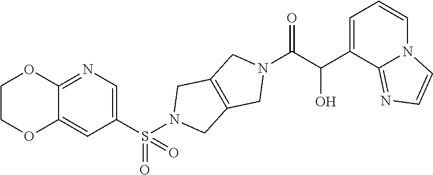 [SMILES: O=C(C(O)c1cccn2ccnc12)N1CC2=C(C1)CN(S(=O)(=O)c1cnc3c(c1)OCCO3)C2] | Examples 103-38 and 103-39 |
| 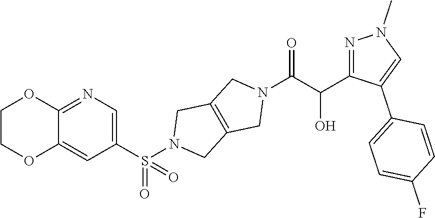 [SMILES: Cn1cc(-c2ccc(F)cc2)c(C(O)C(=O)N2CC3=C(C2)CN(S(=O)(=O)c2cnc4c(c2)OCCO4)C3)n1] | Examples 103-40 and 103-41 |
| 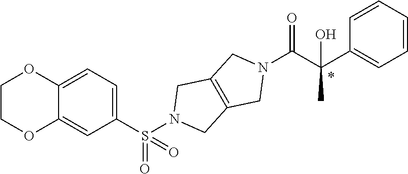 [SMILES: C[C@](O)(C(=O)N1CC2=C(C1)CN(S(=O)(=O)c1ccc3c(c1)OCCO3)C2)c1ccccc1] | Example 103-42 |
| 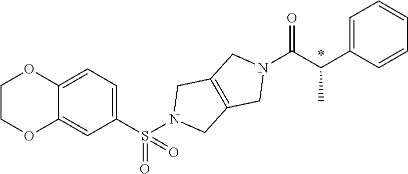 [SMILES: C[C@H](C(=O)N1CC2=C(C1)CN(S(=O)(=O)c1ccc3c(c1)OCCO3)C2)c1ccccc1] | Example 103-43 |
| 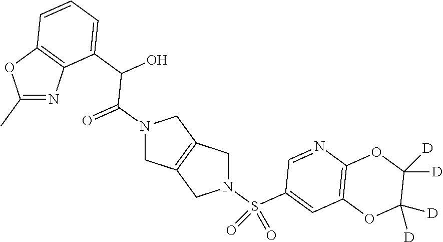 [SMILES: [2H]C1([2H])Oc2cc(S(=O)(=O)N3CC4=C(CN(C(=O)C(O)c5cccc6oc(C)nc56)C4)C3)cnc2OC1([2H])[2H]] | Examples 103-44 and 103-45 |
| 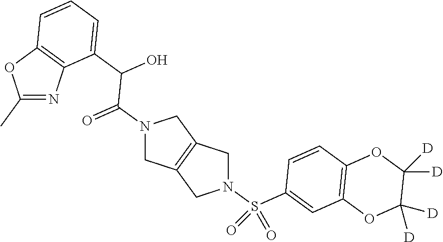 [SMILES: [2H]C1([2H])Oc2ccc(S(=O)(=O)N3CC4=C(CN(C(=O)C(O)c5cccc6oc(C)nc56)C4)C3)cc2OC1([2H])[2H]] | Examples 103-46 and 103-47 |
| 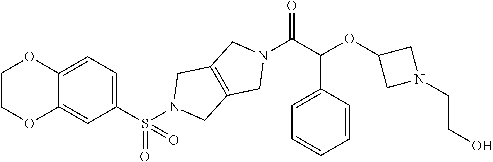 [SMILES: O=C(C(OC1CN(CCO)C1)c1ccccc1)N1CC2=C(C1)CN(S(=O)(=O)c1ccc3c(c1)OCCO3)C2] | Examples 104-1 and 104-2 |
| 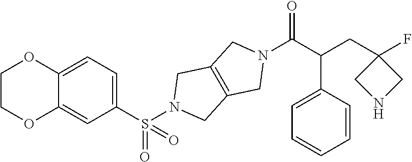 [SMILES: O=C(C(CC1(F)CNC1)c1ccccc1)N1CC2=C(C1)CN(S(=O)(=O)c1ccc3c(c1)OCCO3)C2] | Examples 105-1 and 105-2 |
| 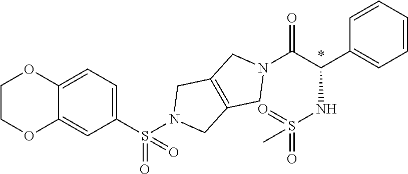 [SMILES: CS(=O)(=O)N[C@H](C(=O)N1CC2=C(C1)CN(S(=O)(=O)c1ccc3c(c1)OCCO3)C2)c1ccccc1] | Example 106-1 |
| 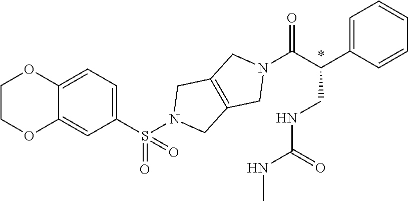 [SMILES: CNC(=O)NC[C@H](C(=O)N1CC2=C(C1)CN(S(=O)(=O)c1ccc3c(c1)OCCO3)C2)c1ccccc1] | Example 106-2 |
| 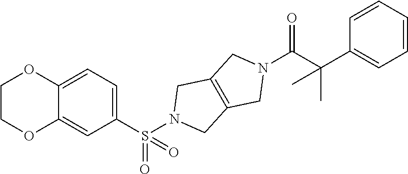 [SMILES: CC(C)(C(=O)N1CC2=C(C1)CN(S(=O)(=O)c1ccc3c(c1)OCCO3)C2)c1ccccc1] | Example 106-3 |
| 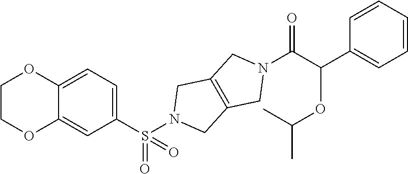 [SMILES: CC(C)OC(C(=O)N1CC2=C(C1)CN(S(=O)(=O)c1ccc3c(c1)OCCO3)C2)c1ccccc1] | Example 106-4 |
| 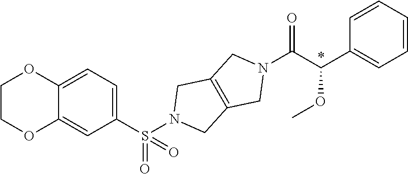 [SMILES: CO[C@H](C(=O)N1CC2=C(C1)CN(S(=O)(=O)c1ccc3c(c1)OCCO3)C2)c1ccccc1] | Example 106-5 |
| 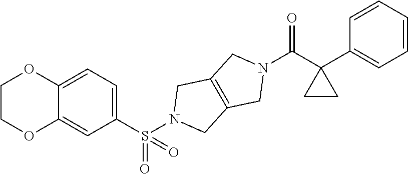 [SMILES: O=C(N1CC2=C(C1)CN(S(=O)(=O)c1ccc3c(c1)OCCO3)C2)C1(c2ccccc2)CC1] | Example 106-6 |
| 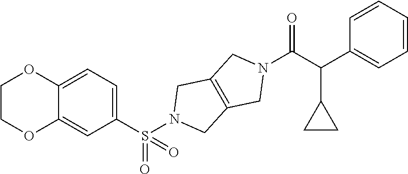 [SMILES: O=C(C(c1ccccc1)C1CC1)N1CC2=C(C1)CN(S(=O)(=O)c1ccc3c(c1)OCCO3)C2] | Example 106-8 |
| 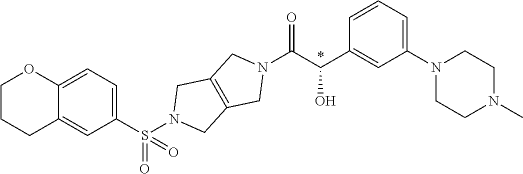 [SMILES: CN1CCN(c2cccc([C@H](O)C(=O)N3CC4=C(C3)CN(S(=O)(=O)c3ccc5c(c3)CCCO5)C4)c2)CC1] | Example 107-1 |
| 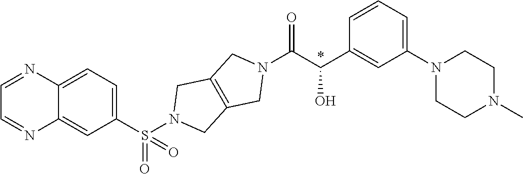 [SMILES: CN1CCN(c2cccc([C@H](O)C(=O)N3CC4=C(C3)CN(S(=O)(=O)c3ccc5nccnc5c3)C4)c2)CC1] | Example 107-2 |
| 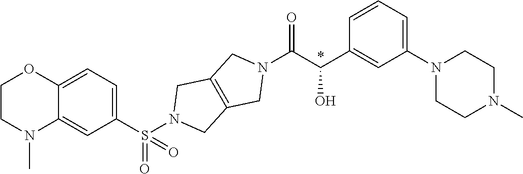 [SMILES: CN1CCN(c2cccc([C@H](O)C(=O)N3CC4=C(C3)CN(S(=O)(=O)c3ccc5c(c3)N(C)CCO5)C4)c2)CC1] | Example 107-3 |
| 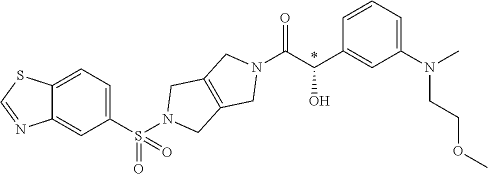 [SMILES: COCCN(C)c1cccc([C@H](O)C(=O)N2CC3=C(C2)CN(S(=O)(=O)c2ccc4scnc4c2)C3)c1] | Example 107-4 |
| 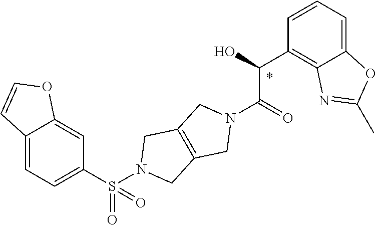 [SMILES: Cc1nc2c([C@H](O)C(=O)N3CC4=C(C3)CN(S(=O)(=O)c3ccc5ccoc5c3)C4)cccc2o1] | Example 108-1 |
| 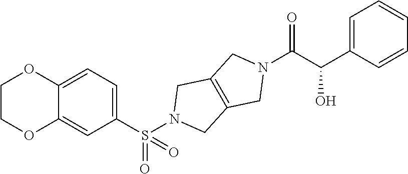 [SMILES: O=C([C@@H](O)c1ccccc1)N1CC2=C(C1)CN(S(=O)(=O)c1ccc3c(c1)OCCO3)C2] | Example 108-2 |
| 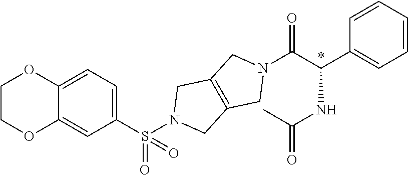 [SMILES: CC(=O)N[C@H](C(=O)N1CC2=C(C1)CN(S(=O)(=O)c1ccc3c(c1)OCCO3)C2)c1ccccc1] | Example 108-3 |
| 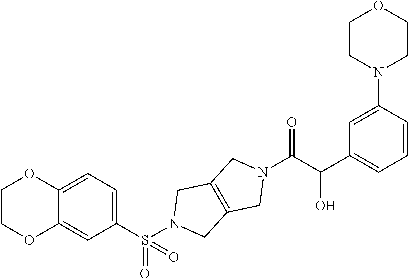 [SMILES: O=C(C(O)c1cccc(N2CCOCC2)c1)N1CC2=C(C1)CN(S(=O)(=O)c1ccc3c(c1)OCCO3)C2] | Example 109-1 |
| 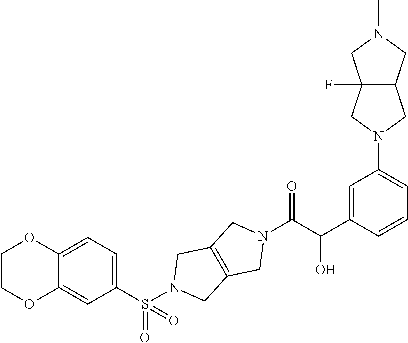 [SMILES: CN1CC2CN(c3cccc(C(O)C(=O)N4CC5=C(C4)CN(S(=O)(=O)c4ccc6c(c4)OCCO6)C5)c3)CC2(F)C1] | Examples 109-2, 109-3, 109-4, and 109-5 |
| 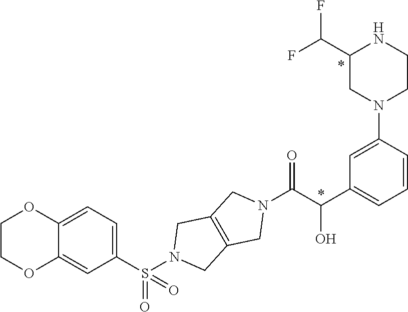 [SMILES: O=C(C(O)c1cccc(N2CCNC(C(F)F)C2)c1)N1CC2=C(C1)CN(S(=O)(=O)c1ccc3c(c1)OCCO3)C2] | Examples 109-6 and 109-7 |
| 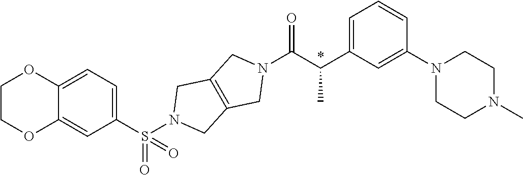 [SMILES: C[C@H](C(=O)N1CC2=C(C1)CN(S(=O)(=O)c1ccc3c(c1)OCCO3)C2)c1cccc(N2CCN(C)CC2)c1] | Example 109-8 |
| 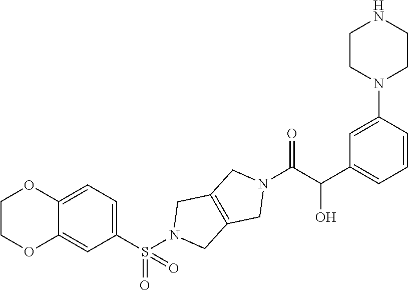 [SMILES: O=C(C(O)c1cccc(N2CCNCC2)c1)N1CC2=C(C1)CN(S(=O)(=O)c1ccc3c(c1)OCCO3)C2] | Example 109-9 |
| 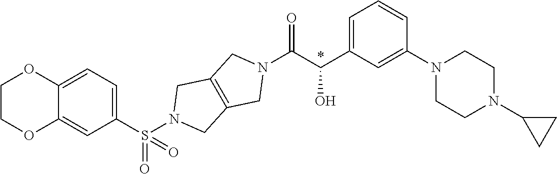 [SMILES: O=C([C@@H](O)c1cccc(N2CCN(C3CC3)CC2)c1)N1CC2=C(C1)CN(S(=O)(=O)c1ccc3c(c1)OCCO3)C2] | Example 109-10 |
| 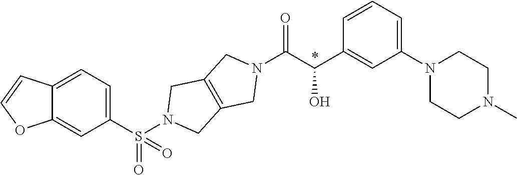 [SMILES: CN1CCN(c2cccc([C@H](O)C(=O)N3CC4=C(C3)CN(S(=O)(=O)c3ccc5ccoc5c3)C4)c2)CC1] | Example 109-11 |
| 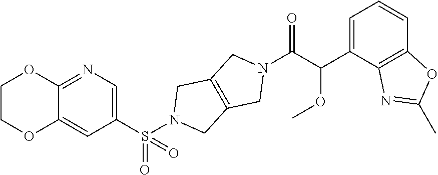 [SMILES: COC(C(=O)N1CC2=C(C1)CN(S(=O)(=O)c1cnc3c(c1)OCCO3)C2)c1cccc2oc(C)nc12] | Examples 110-1 and 110-2 |
| 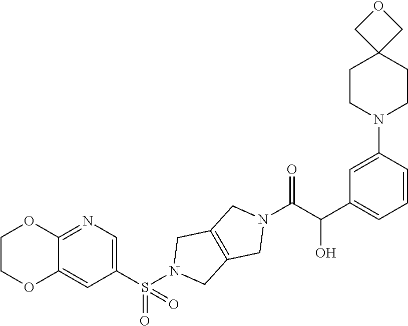 [SMILES: O=C(C(O)c1cccc(N2CCC3(CC2)COC3)c1)N1CC2=C(C1)CN(S(=O)(=O)c1cnc3c(c1)OCCO3)C2] | Examples 111-1 and 111-2 |
| 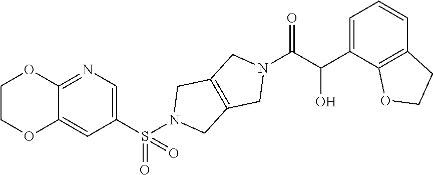 [SMILES: O=C(C(O)c1cccc2c1OCC2)N1CC2=C(C1)CN(S(=O)(=O)c1cnc3c(c1)OCCO3)C2] | Examples 112-1 and 112-2 |
| 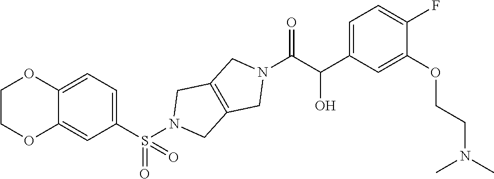 [SMILES: CN(C)CCOc1cc(C(O)C(=O)N2CC3=C(C2)CN(S(=O)(=O)c2ccc4c(c2)OCCO4)C3)ccc1F] | Examples 112-3 and 112-4 |
| 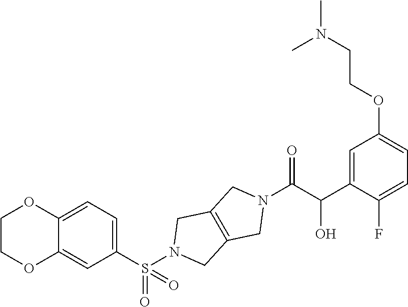 [SMILES: CN(C)CCOc1ccc(F)c(C(O)C(=O)N2CC3=C(C2)CN(S(=O)(=O)c2ccc4c(c2)OCCO4)C3)c1] | Examples 112-5 and 112-6 |
| 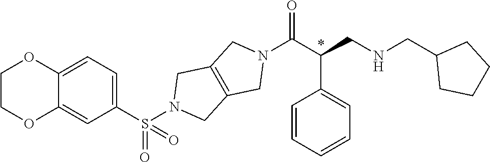 [SMILES: O=C([C@@H](CNCC1CCCC1)c1ccccc1)N1CC2=C(C1)CN(S(=O)(=O)c1ccc3c(c1)OCCO3)C2] | Example 113-1 |
| 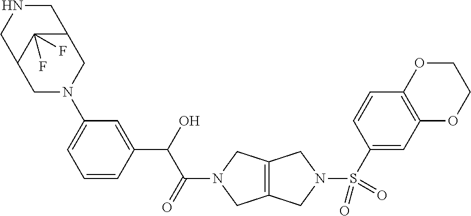 [SMILES: O=C(C(O)c1cccc(N2CC3CNCC(C2)C3(F)F)c1)N1CC2=C(C1)CN(S(=O)(=O)c1ccc3c(c1)OCCO3)C2] | Examples 114-1 and 114-2 |
| 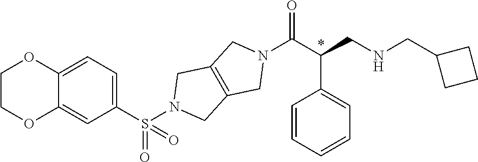 [SMILES: O=C([C@@H](CNCC1CCC1)c1ccccc1)N1CC2=C(C1)CN(S(=O)(=O)c1ccc3c(c1)OCCO3)C2] | Example 114-3 |
| 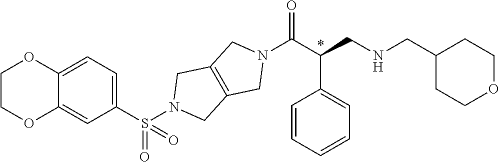 [SMILES: O=C([C@@H](CNCC1CCOCC1)c1ccccc1)N1CC2=C(C1)CN(S(=O)(=O)c1ccc3c(c1)OCCO3)C2] | Example 114-4 |
| 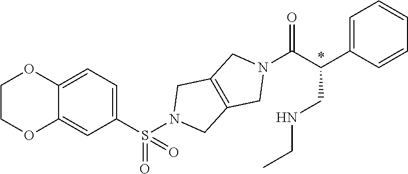 [SMILES: CCNC[C@H](C(=O)N1CC2=C(C1)CN(S(=O)(=O)c1ccc3c(c1)OCCO3)C2)c1ccccc1] | Example 114-5 |
| 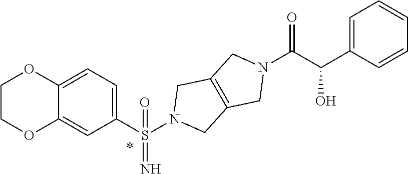 [SMILES: N=S(=O)(c1ccc2c(c1)OCCO2)N1CC2=C(CN(C(=O)[C@@H](O)c3ccccc3)C2)C1] | Example 115 |

*Absolute stereochemistry not determined.

It will be appreciated that throughout the present disclosure, unless otherwise indicated, reference to a compound of Formula I is intended to also include I, I-a, I-b, II, II-a, II-b, II-c, III, III-a, III-b, III-e, IV, IV-a, IV-b, V, V-a, and V-b, and compound species of such formulas disclosed herein.

Unless otherwise stated, it will be appreciated that when “one or more” substituents are recited for a particular variable, it includes one, two, three, four, or more substituents as valency permits.

In some embodiments of any Formula disclosed herein, a heterocyclyl at the R¹, R², R^{1′}, R^{2′}, or B position does not contain an O in the γ-position relative to C(═Z¹) or C(═O). In some embodiments of any Formula disclosed herein, a heterocyclyl at the R¹, R², R^{1′}, R^{2′}, or B position contains 1-4 heteroatoms independently selected from the group consisting of N and S. In some embodiments of any Formula disclosed herein, when R^{e} is —OR, —OR does not result in an O in the γ-position relative to C(═Z¹),

Unless otherwise stated, structures depicted herein are also meant to include all stereoisomeric (e.g., enantiomeric or diastereomeric) forms of the structure, as well as all geometric or conformational isomeric forms of the structure; for example, the R and S configurations for each stereocenter. Therefore, single stereochemical isomers, as well as enantiomeric, diastereomeric, and geometric (or conformational) mixtures of the present compounds are within the scope of the disclosure. For example, in some cases Table 1 shows one or more stereoisomers of a compound, and unless otherwise indicated, represents each stereoisomer alone and/or as a mixture. Unless otherwise stated, all tautomeric forms of the compounds of the disclosure are within the scope of the disclosure.

In some embodiments, a compound of Formula I is obtained by a process comprising a purification method in Table 21. In some embodiments, the compound is obtained by a process comprising a purification method in Table 21 and is the 1^{st} eluting isomer of the purification method. In some embodiments, the compound is obtained by a process comprising a purification method in Table 21 and is the 2^{nd} eluting isomer of the purification method. In some embodiments, the compound is obtained by a process comprising a purification method in Table 21 and is the 3^{rd} eluting isomer of the purification method. In some embodiments, the compound is obtained by a process comprising a purification method in Table 21 and is the 4^{th} eluting isomer of the purification method. In some embodiments, the compound is obtained by a process comprising a purification method in Table 21 and is the 5^{th}, 6^{th}, 7^{th} or 8^{th} eluting isomer of the purification method.

In some embodiments, a USP9X Inhibitor is obtained by a process comprising a purification method in Table 21. In some embodiments, the USP9X Inhibitor is obtained by a process comprising a purification method in Table 21 and is the 1^{st} eluting isomer of the purification method. In some embodiments, the USP9X Inhibitor is obtained by a process comprising a purification method in Table 21 and is the 2^{nd} eluting isomer of the purification method. In some embodiments, the USP9X Inhibitor is obtained by a process comprising a purification method in Table 21 and is the 3^{rd} eluting isomer of the purification method. In some embodiments, the USP9X Inhibitor is obtained by a process comprising a purification method in Table 21 and is the 4^{th} eluting isomer of the purification method. In some embodiments, the USP9X Inhibitor is obtained by a process comprising a purification method in Table 21 and is the 5^{th}, 6^{th}, 7^{th} or 8^{th} eluting isomer of the purification method.

Additionally, unless otherwise stated, structures depicted herein are also meant to include compounds that differ only in the presence of one or more isotopically enriched atoms. For example, compounds having the present structures including the replacement of hydrogen by deuterium or tritium (e.g., Examples 103-44, 103-45, 103-46, and 103-47), or the replacement of a carbon by a ¹³C- or ¹⁴C-enriched carbon are within the scope of this disclosure.

In some embodiments, prodrugs of the compounds disclosed herein are provided. As used herein, the term “prodrug” refers to a compound that is a drug precursor which, following administration, releases the drug in vivo via a chemical or physiological process (e.g., a prodrug releases the drug upon reaching physiological pH or through enzyme action is converted to the desired drug form). Prodrugs can be obtained by including a group on the compound to increase solubility or bioabsorption (e.g., a phosphate group). In one example, the prodrug group is a phosphate group, which can be attached to a compound of Formula I at R¹, when R² is H, wherein upon administration, the prodrug is metabolized to form a compound of Formula I. In some embodiments, a compound of Formula I is formed as a metabolite of a prodrug.

The disclosure also provides compounds of Formula I (e.g., compounds that are not USP9X Inhibitors) that are useful, for example, as analytical tools and/or control compounds in biological assays.

The compounds of Formula I may form salts which are also within the scope of this disclosure. Reference to a compound of the Formula I herein is understood to include reference to salts thereof, unless otherwise indicated. Pharmaceutically acceptable salts are well known in the art. For example, S. M. Berge, et al. describes pharmaceutically acceptable salts in detail in J. Pharmaceutical Sciences, 66: 1-19 (1977).

The disclosure also includes pharmaceutical compositions comprising one or more compounds as described herein, or a pharmaceutically acceptable salt thereof, and a pharmaceutically acceptable excipient. In some embodiments, pharmaceutical compositions reported herein can be provided in a unit dosage form (e.g., capsule, tablet or the like). In some embodiments, pharmaceutical compositions reported herein can be provided in an oral dosage form. In some embodiments, the pharmaceutical composition is orally administered in any orally acceptable dosage form. In some embodiments, an oral dosage form of a compound of Formula I can be a capsule. In some embodiments, an oral dosage form of a compound of Formula I is a tablet. In some embodiments, an oral dosage form comprises one or more fillers, disintigrants, lubricants, glidants, anti-adherents and/or anti-statics. In some embodiments, an oral dosage form is prepared via dry blending. In some embodiments, an oral dosage form is a tablet and is prepared via dry granulation.

The designations “α”, “β”, “γ”, “δ”, “ε”, etc. are used herein to refer to a position in a molecule relative to a carbonyl group, in accordance with standard nomenclature. For example, a carbon in the α-position (or an α-carbon) is a carbon atom in the position adjacent to a carbonyl group; and an oxygen in the β-position (or a β-oxygen) is an oxygen atom in the position two atoms away from a carbonyl group. The scheme below illustrates this nomenclature on an exemplary compound:

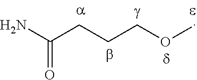

### Methods of Using the Disclosed Compounds

Another aspect of the present disclosure is the use of compounds of Formula I. Compounds of Formula I are useful in medicine. For example, compounds and compositions described herein are inhibitors of USP9X. Methods of treatment (e.g., by inhibiting USP9X) can comprise administering to a subject in need thereof a therapeutically effective amount of (i) a compound disclosed herein, or a pharmaceutically acceptable salt thereof or (ii) a pharmaceutical composition comprising a compound disclosed herein, or a pharmaceutically acceptable salt thereof, and a pharmaceutically acceptable carrier. In some embodiments, a method of treating a disease associated with modulation of USP9X comprises administering a therapeutically effective amount of a compound disclosed herein. In some embodiments, a method of treating cancer comprises administering a therapeutically effective amount of a compound disclosed herein.

### Methods of Synthesizing the Disclosed Compounds

The compounds of the present disclosure may be made by a variety of methods, including standard chemistry. Suitable synthetic routes are depicted in the Schemes given below.

### EXAMPLES

### Analytical Methods, Materials, and Instrumentation

Unless otherwise noted, reagents and solvents were used as received from commercial suppliers. Unless otherwise noted, reactions were conducted under an inert atmosphere of nitrogen. NMR instrument: Bruker BBFO ASCEND™ 400 AVANCE III 400 MHz and Bruker BBFO ULTRASHIELD™ 300 AVANCE III 300 MHz. Internal standard: Tetramethylsilane (TMS). MassSpec instruments and ionization method: Shimadzu LC-2020, electrospray ionization, ESI. Chromatography instruments (Reverse phase chromatography: Agela TechnologiesMP200. Preparatory HPLC (Prep-HPLC): Waters. Supercritical fluid chromatography (SFC): Shimadzu).

### Abbreviations Used Herein

Ac₂O Acetic anhydride
ACN Acetonitrile
AcOH Acetic acid
AIBN 2,2′-Azobis(2-methylpropionitrile)
BOP (Benzotriazol-1-yloxy)tris(dimethylamino)phosphonium hexafluorophosphate
CDI 1,1′-Carbonyldiimidazole
δ chemical shift
DCM Dichloromethane or methylene chloride
DCE 1,2-Dichloroethane
DEAD Diethyl azodicarboxylate
DIAD Diisopropyl azodicarboxylate
DIEA N,N-Diisopropylethylamine
DMA N,N-Dimethylacetamide
DMAP 4-dimethylaminopyridine
DME Dimethoxyethane
DMF N,N-Dimethylformamide
DMP Dess-Martin Periodinane
DMSO Dimethylsulfoxide
dppf 1,1′-Bis(diphenylphosphino)ferrocene
EDCI N-(3-Dimethylaminopropyl)-N′-ethylcarbodiimide hydrochloride
EDTA Ethylenediaminetetraacetic acid
ee enantiomeric excess
h hour
¹H NMR proton nuclear magnetic resonance
HATU 2-(3H-[1,2,3]Triazolo[4,5-b]pyridin-3-yl)-1,1,3,3-tetramethylisouronium hexafluorophosphate
HOBT 1H-Benzo[d][1,2,3]triazol-1-ol hydrate
HPLC high performance liquid chromatography
Hz Hertz
IPA Isopropyl alcohol
LAH lithium aluminum hydride
LCMS liquid chromatography/mass spectrometry
m-CPBA m-Chloroperoxybenzoic acid
MeOH Methanol
min minutes
MS mass spectrometry
NMM 4-Methylmorpholine
NMP N-Methyl-2-pyrrolidone
Pd(dppf)Cl₂ [1,1′-Bis(diphenylphosphino)ferrocene]dichloropalladium(II)
Pd(PPh₃)₄ Tetratkis(triphenylphosphine)palladium (0)
PTSA 4-methylbenzenesulfonic acid
rt room temperature
Rt retention time
RuPhos 2-Dicyclohexylphosphino-2′,6′-diisopropoxybiphenyl
RuPhos 2G Chloro(2-dicyclohexylphosphino-2′,6′-diisopropoxy-1,1′-biphenyl)[2-(2′-amino-1,1′-biphenyl)]palladium(II)
TBDMS-Cl tert-butyl dimethylsilyl chloride
TEA Triethylamine
TFA Trifluoroacetic acid
THF Tetrahydrofuran
TLC thin layer chromatography
TMSCN Trimethylsilyl cyanide
Xantphos 4,5-Bis(diphenylphosphino)-9,9-dimethylxanthene
XPhos 2-Dicyclohexylphosphino-2′,4′,6′-triisopropylbiphenyl
XPhos 2G Chloro(2-dicyclohexylphosphino-2′,4′,6′-triisopropyl-1,1′-biphenyl)[2-(2′-amino-1,1′-biphenyl)]palladium(II)
XPhos 3G Methanesulfonato(2-dicyclohexylphosphino-2′,4′,6′-tri-i-propyl-1,1′-biphenyl)(2′-amino-1,1′-biphenyl-2-yl)palladium(II) dichloromethane adduct
STAB Sodium triacetoxyborohydride
PFA Paraformaldehyde

### Example 1. Biochemical Assay for USP9X Inhibition

The assay was performed in a final volume of 6 μL assay buffer containing 20 mM Tris-HCl (pH 8.0, (1M Tris-HCl, pH 8.0 solution; Corning 46-031-CM)), L-Glutathione (GSH) reducing agent (1 mM, Sigma-Aldrich, G4251-100G), 0.03% Bovine Gamma Globulin (BGG) (0.22 μM filtered, Sigma, G7516-25G), and 0.01% Triton X-100 (Sigma, T9284-10L). DMSO solutions of the compounds in nanoliter quantities (10-point, 3-fold serial dilutions) were dispensed into 1536 assay plates (Corning, #3724BC) for final test concentrations of 25 μM to 1.3 nM, top to lowest dose, respectively. Concentration and incubation times were optimized for the maximal signal-to-background while maintaining initial velocity conditions at a fixed substrate concentration (<<Kₘ). The final concentration of USP9X (Enzyme, E) was 0.025 nM, and the final concentration of Ubiquitin-Rhoadmine 110 (Ub-Rh110, UbiQ-126) (Substrate, S) was 25 nM. To assay plates (pre-stamped with compound) was added 3 μL 2× Enzyme. The enzyme was preincubated for 30 minutes and then treated with 3 μL of 2× Substrate. Plates were incubated for 11 min (continuous kinetic read) at room temperature before the fluorescence was read on the Envision plate reader (Perkin Elmer) or PheraSTAR plate reader (BMG), with excitation at 485 nm and emission at 535 nm. The slope (best fit linear regression) of the five reads was used to normalize for inhibition. For all assays, data are reported as percent inhibition compared with control wells based on the following equation: % inh=100*((FLU−AveLow)/(AveHigh−AveLow)), wherein FLU is measured Fluorescence, AveLow is average Fluorescence of no enzyme control (n=64), and AveHigh is average Fluorescence of DMSO control (n=64). IC₅₀ values are determined by curve fitting of the standard 4 parameter logistic fitting algorithm included in the Activity Base software package: IDBS XE Designer Model205. Data are fitted using the Levenburg Marquardt algorithm.

As set forth in Tables 22 and 23, IC₅₀ values are defined as follows: ≤25 μM and >2 μM (+); ≤2 μM and >0.2 μM (++); ≤0.2 μM and >0.05 μM (+++); ≤0.05 μM and >0.001 μM (++++); and not tested (−−).

### Synthesis of Intermediates

### Intermediate 2-1. 5-(2,3-dihydro-1,4-benzodioxine-6-sulfonyl)-1H,2H,3H,5H-pyrrolo[3,4-c]pyrrole TFA Salt

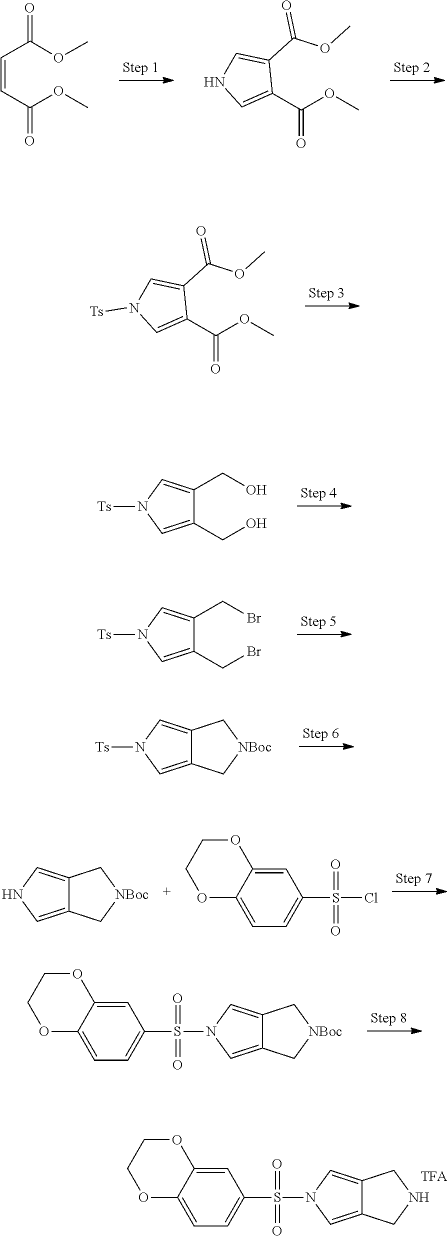

### Step 1. 3,4-dimethyl 1H-pyrrole-3,4-dicarboxylate

To a solution of 1,4-dimethyl (2Z)-but-2-enedioate (7.4 g, 51.3 mmol) in tetrahydrofuran (1 L) was added tosylmethyl isocyanide (10 g, 51.5 mmol) followed by the addition of t-BuOK (11.6 g, 103 mmol) in portions with stirring at 0° C. The resulting mixture was stirred for 2 h at room temperature. The reaction mixture was poured into brine (500 mL) and then extracted with ethyl acetate (2×250 mL). The combined organic layers were dried over anhydrous sodium sulfate, filtered, and concentrated under vacuum. The solids were treated with ethyl acetate, filtered and dried under vacuum to afford 3,4-dimethyl 1H-pyrrole-3,4-dicarboxylate as a tan solid (5.5 g, 58%). LCMS (ES, m/z) 184 [M+H]⁺.

### Step 2. 3,4-dimethyl 1-(4-methylbenzenesulfonyl)-1H-pyrrole-3,4-dicarboxylate

To a solution of 3,4-dimethyl 1H-pyrrole-3,4-dicarboxylate (5.5 g, 30 mmol) in DMF (100 mL) was added sodium hydride (1.1 g, 45.1 mmol, 60% dispersion in mineral oil) in portions with stirring at 0° C. The resulting solution was stirred for 0.5 h at room temperature. A solution of 4-toluene sulfonyl chloride (6.9 g, 36.2 mmol) in DMF (10 mL) was added slowly at 0° C. and the resulting mixture was stirred for 2 h at room temperature. The reaction mixture was poured into saturated ammonium chloride solution (50 mL) and then extracted with ethyl acetate (3×200 mL). The combined organic layers were dried over anhydrous sodium sulfate, filtered, and concentrated under vacuum. The resulting crude product was purified by silica gel chromatography (eluting with 0:100 to 33:66 ethyl acetate/petroleum ether) to afford 3,4-dimethyl 1-(4-methylbenzenesulfonyl)-1H-pyrrole-3,4-dicarboxylate as a white solid (4.0 g, 39%). LCMS (ES, m/z) 338 [M+H]⁺.

### Step 3. [4-(hydroxymethyl)-1-(4-methylbenzenesulfonyl)-1H-pyrrol-3-yl]methanol

To a solution of 3,4-dimethyl 1-(4-methylbenzenesulfonyl)-1H-pyrrole-3,4-dicarboxylate (4.0 g, 11.9 mmol) in tetrahydrofuran (50 mL) was added lithium aluminum hydride (900 mg, 23.7 mmol) in portions with stirring at 0° C. The resulting solution was stirred for 2 h at room temperature. The reaction was quenched by careful addition of sodium sulfate 10H₂O. The resulting mixture was filtered and concentrated under vacuum and purified by silica gel chromatography (eluting with 0:100 to 33:66 ethyl acetate/petroleum ether) to afford [4-(hydroxymethyl)-1-(4-methylbenzenesulfonyl)-1H-pyrrol-3-yl]methanol as a dark red solid (2.5 g, 75%). LCMS (ES, m/z) 264 [M+H—H₂O]⁺.

### Step 4. 3,4-bis(bromomethyl)-1-(4-methylbenzenesulfonyl)-1H-pyrrole

To a solution of [4-(hydroxymethyl)-1-(4-methylbenzenesulfonyl)-1H-pyrrol-3-yl]methanol (2.5 g, 8.89 mmol) in dichloromethane (30 mL) was added tribromophosphane (4.8 g, 17.7 mmol) dropwise with stirring at 0° C. The resulting mixture was stirred for 2 h at room temperature. The reaction mixture was poured into saturated aqueous sodium bicarbonate (30 mL) and then extracted with ethyl acetate (3×50 mL). The combined organic layers were dried over anhydrous sodium sulfate, filtered and concentrated under vacuum. The resulting crude product was purified by silica gel chromatography (eluting with 0:100 to 20:80 ethyl acetate/petroleum ether) to afford 3,4-bis(bromomethyl)-1-(4-methylbenzenesulfonyl)-1H-pyrrole as a dark red solid (1.9 g, 53%). LCMS (ES, m/z) 408, 406, 410 [M+H]⁺.

### Step 5. 3,4-bis(bromomethyl)-1-(4-methylbenzenesulfonyl)-1H-pyrrole

To a solution of tert-butyl carbamate (0.8 g, 6.8 mmol) in DMF (20 mL) was added sodium hydride (0.4 g, 10.0 mmol, 60% dispersion in mineral oil) in portions with stirring at 0° C. The resulting solution was stirred for 0.5 h at room temperature. 3,4-bis(bromomethyl)-1-(4-methylbenzenesulfonyl)-1H-pyrrole (1.8 g, 4.42 mmol) was added and the resulting mixture was stirred for 2 h at room temperature. The solution was poured into water (20 mL). The solids were collected by filtration and dried under vacuum to afford 3,4-bis(bromomethyl)-1-(4-methylbenzenesulfonyl)-1H-pyrrole as a yellow solid (1.4 g, 69%). LCMS (ES, m/z) 348 [M+H—CH₃]⁺.

### Step 6. Tert-butyl 1H,2H,3H,5H-pyrrolo[3,4-c]pyrrole-2-carboxylate

To a solution of 3,4-bis(bromomethyl)-1-(4-methylbenzenesulfonyl)-1H-pyrrole (700 mg, 1.93 mmol) in methanol (15 mL) was added sodium hydroxide (1.2 g, 30.0 mmol). The resulting solution was stirred for 3 h at 65° C. and then cooled to room temperature. The reaction mixture was poured into water (20 mL) and then extracted with ethyl acetate (3×30 mL). The combined organic layers were dried over anhydrous sodium sulfate, filtered and concentrated under vacuum to afford tert-butyl 1H,2H,3H,5H-pyrrolo[3,4-c]pyrrole-2-carboxylate as yellow solid (300 mg, crude). LCMS (ES, m/z) 153 [M+H-t-Bu]⁺.

### Step 7. Tert-butyl 5-(2,3-dihydro-1,4-benzodioxine-6-sulfonyl)-1H,2H,3H,5H-pyrrolo[3,4-c]pyrrole-2-carboxylate

To a solution of tert-butyl 1H,2H,3H,5H-pyrrolo[3,4-c]pyrrole-2-carboxylate (250 mg, 1.20 mmol) in dichloromethane (3 mL) was added sodium hydroxide (61 mg, 1.53 mmol) and Bu₄NHSO₄ (41 mg, 0.12 mmol). A solution of 2,3-dihydro-1, 4-benzodioxine-6-sulfonyl chloride (340 mg, 1.20 mmol) in dichloromethane (3 mL) was added dropwise with stirring at 0° C. The resulting mixture was stirred for 3 h at room temperature. The reaction mixture was poured into saturated ammonium chloride (5 mL) and then extracted with ethyl acetate (3×20 mL). The combined organic layers were dried over anhydrous sodium sulfate, filtered and concentrated under vacuum. The resulting crude product was purified by Prep-TLC (eluting with 3:1 ethyl acetate/petroleum ether) to afford tert-butyl 5-(2,3-dihydro-1,4-benzodioxine-6-sulfonyl)-1H,2H,3H,5H-pyrrolo[3,4-c]pyrrole-2-carboxylate as brown oil (200 mg, 49%). LCMS (ES, m/z) 392 [M+H—CH₃]⁺.

### Step 8. 5-(2,3-dihydro-1,4-benzodioxine-6-sulfonyl)-1H,2H,3H,5H-pyrrolo[3,4-c]pyrrole TFA Salt

To a solution of tert-butyl 5-(2,3-dihydro-1,4-benzodioxine-6-sulfonyl)-1H,2H,3H,5H-pyrrolo[3,4-c]pyrrole-2-carboxylate (200 mg, 0.49 mmol) in dichloromethane (3 mL) was added TFA (0.6 mL). The resulting solution was stirred for 1 h at room temperature. The resulting mixture was concentrated under vacuum to afford 5-(2,3-dihydro-1,4-benzodioxine-6-sulfonyl)-1H,2H,3H,5H-pyrrolo[3,4-c]pyrrole TFA salt as yellow oil (190 mg, crude). LCMS (ES, m/z) 307 [M+H]⁺.

### Intermediate 2-2. 2-((2,3-dihydrobenzo[b][1,4]dioxin-6-yl)sulfonyl)-1,2,3,4,5,6-hexahydropyrrolo[3,4-c]pyrrole

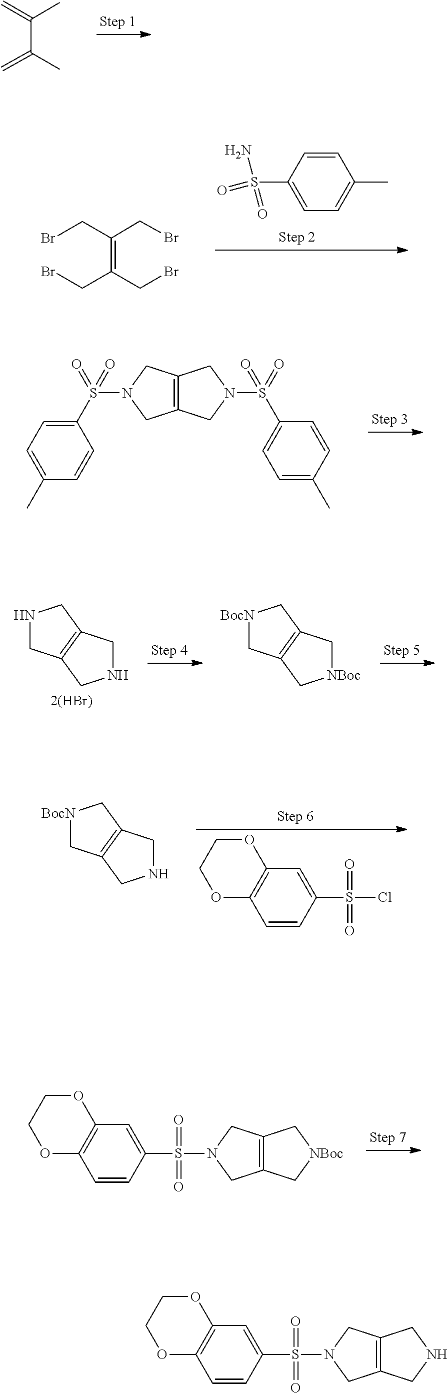

### Step 1. 1,4-dibromo-2,3-bis(bromomethyl)but-2-ene

To a solution of 2,3-dimethylbut-2-ene (1000 g, 11.9 mol) in DCM (1000 mL) in a 4 L 4-necked round bottom flask was added aqueous hydrogen bromide solution (150 mL, 48%) with stirring at 10-15° C. To the reaction was added bromine (9.90 kg, 62.0 mol) with stirring at 0° C. The resulting mixture was stirred for 2 days at 45° C. in an oil bath. After cooling to room temperature, the reaction mixture was carefully poured into saturated aqueous sodium hydrogen sulfite solution (10 L). The precipitate was collected by filtration and dried in oven to afford 1,4-dibromo-2,3-bis(bromomethyl)but-2-ene as a light yellow solid (3000 g, 44%). GCMS: (EI, m/z): 398, 400, 402 [M]⁺.

### Step 2. 2,5-ditosyl-1,2,3,4,5,6-hexahydropyrrolo[3,4-c]pyrrole

To a solution of 1,4-dibromo-2,3-bis(bromomethyl)but-2-ene (2000 g, 3.50 mol) in DMF (20 L) was added 4-methylbenzene-1-sulfonamide (2137 g, 12.5 mol), and potassium carbonate (5175 g, 37.4 mol). The resulting mixture was stirred for 2 days at room temperature. The reaction mixture was slowly poured into water/ice (20 L). The precipitate was collected by filtration, washed with ethanol and dried in an oven to afford 2,5-ditosyl-1,2,3,4,5,6-hexahydropyrrolo[3,4-c]pyrrole as a light yellow solid (1345 g, 78%). LCMS: (ES, m/z): 419 [M+H]⁺.

### Step 3. 1,2,3,4,5,6-hexahydropyrrolo[3,4-c]pyrrole Hydro Bromide Salt

To a solution of 2,5-ditosyl-1,2,3,4,5,6-hexahydropyrrolo[3,4-c]pyrrole (1345 g, 2.73 mol) in aqueous hydrogen bromide solution (4500 mL, 48%) in 10 L 4-necked round-bottom flask, was added phenol (1270 g, 13.5 mol). The resulting mixture was stirred for 2 days at 120° C. After cooling to room temperature, the aqueous layer was collected and concentrated under vacuum. The resulting solids were washed with DCM/MeOH (v:v=10:1, 3×300 mL) and dried in an oven to afford 1,2,3,4,5,6-hexahydropyrrolo[3,4-c]pyrrole hydrogen bromide salt as a yellow solid (480 g, 61%). LCMS: (ES, m/z): 111 [M+H]⁺.

### Step 4. Di-tert-butyl pyrrolo[3,4-c]pyrrole-2,5(1H,3H,4H,6H)-dicarboxylate

To a suspension of 1,2,3,4,5,6-hexahydropyrrolo[3,4-c]pyrrole hydrogen bromide salt (458 g, 1.52 mol) in water (4 L) was added sodium bicarbonate (424 g, 5.05 mol) followed by dropwise addition of a solution of di-tert-butyl dicarbonate (807 g, 3.70 mol) in methanol (500 mL) with stirring at 0° C. The resulting solution was stirred for 16 h at 25° C. The precipitate was collected by filtration and dried in an oven to afford di-tert-butyl pyrrolo[3,4-c]pyrrole-2,5(1H,3H,4H,6H)-dicarboxylate as a white solid (300 g, 61%). LCMS (ES, m/z): 311[M+H]⁺.

### Step 5. Tert-butyl 4,5-dihydropyrrolo[3,4-c]pyrrole-2(1H,3H,4H)-carboxylate 4-methylbenzene-1-sulfonic Acid Salt

To a solution of di-tert-butyl pyrrolo[3,4-c]pyrrole-2,5(1H,3H,4H,6H)-dicarboxylate (200 g, 612 mmol) in propan-2-yl acetate (5 L) was added 4-methylbenzene-1-sulfonic acid (123 g, 647 mmol) in portions at 0° C. The resulting mixture was stirred for 16 h at 55° C. in an oil bath. After cooling to room temperature, the precipitate was collected by filtration and dried in an oven to afford tert-butyl 4,5-dihydropyrrolo[3,4-c]pyrrole-2(1H,3H,4H)-carboxylate 4-methylbenzene-1-sulfonic acid salt as a yellow solid (197 g, 80%). LCMS: (ES, m/z): 211[M+H]⁺.

### Step 6. Tert-butyl 5-(2,3-dihydrobenzo[b][1,4]dioxin-6-ylsulfonyl)-4,5-dihydropyrrolo[3,4-c]pyrrole-2(1H,3H,4H)-carboxylate

To a suspension of tert-butyl 4,5-dihydropyrrolo[3,4-c]pyrrole-2(1H,3H,4H)-carboxylate 4-methylbenzene-1-sulfonic acid salt (61 g, 142 mmol) in water (100 mL) and tetrahydrofuran (30 mL) was added sodium hydroxide (13 g, 325 mmol) followed by portion-wise addition of 2,3-dihydro-1,4-benzodioxine-6-sulfonyl chloride (25 g, 95.9 mmol) at 0° C. The resulting mixture was stirred for 2 h at 25° C. The product was extracted with ethyl acetate (3×200 mL). The organic layers were combined, dried over anhydrous sodium sulfate, filtered and concentrated under vacuum. The resulting material was purified by silica gel chromatography (eluting with 1:10 ethyl acetate/petroleum ether) to afford tert-butyl 5-(2,3-dihydrobenzo[b][1,4]dioxin-6-ylsulfonyl)-4,5-dihydropyrrolo[3,4-c]pyrrole-2(1H,3H,4H)-carboxylate as a white solid (30 g, 73%). LCMS: (ES, m/z): 409 [M+H]⁺.

### Step 7. 2-(2,3-dihydrobenzo[b][1,4]dioxin-6-ylsulfonyl)-1,2,3,4,5,6-hexahydropyrrolo[3,4-c]pyrrole Hydrochloric Salt

To a solution of tert-butyl 5-(2,3-dihydrobenzo[b][1,4]dioxin-6-ylsulfonyl)-4,5-dihydropyrrolo[3,4-c]pyrrole-2(1H,3H,4H)-carboxylate (30.0 g, 69.8 mmol) in 1,4-dioxane (100 mL) was added hydrochloric acid (200 mL, 4 M in 1,4-dioxane). The resulting solution was stirred for 2 h at 25° C. and then concentrated under vacuum to afford 2-(2,3-dihydrobenzo[b][1,4]dioxin-6-ylsulfonyl)-1,2,3,4,5,6-hexahydropyrrolo[3,4-c]pyrrole hydrochloric salt as a yellow solid (20 g, 79%). LCMS: (ES, m/z): 309 [M+H]⁺.

### Intermediate 2-3. methyl 2-(3-cyclopropyl-4-methoxyphenyl)acetate

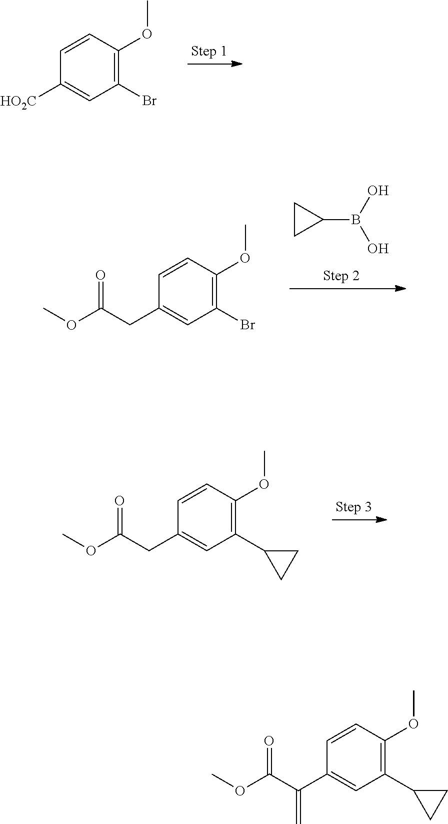

### Step 1. methyl 2-(3-cyclopropyl-4-methoxyphenyl)prop-2-enoate

To a solution of methyl 2-(3-cyclopropyl-4-methoxyphenyl) acetate (840 mg, 3.82 mmol) in N,N-dimethylformamide (25 mL) was added paraformaldehyde (388 mg, 4.31 mmol), tetrabutylazanium iodide (133 mg, 0.36 mmol), and potassium carbonate (1.25 g, 9.0 mmol). The resulting solution was stirred for 10 min at 60° C. and then cooled to room temperature. The reaction mixture was poured into water (50 mL) and extracted with ethyl acetate (3×80 mL). The combined organic layers were dried over anhydrous sodium sulfate, filtered, and concentrated under vacuum. The resulting crude product was purified by silica gel chromatography (eluting with 0:100 to 4:96 ethyl acetate/petroleum ether) to afford methyl 2-(3-cyclopropyl-4-methoxyphenyl)prop-2-enoate as yellow oil (300 mg, 34%). LCMS (ES, m/z) 233 [M+H]⁺.

### Step 2. methyl 2-(3-bromo-4-methoxyphenyl)acetate

To a solution of methyl 2-(3-bromo-4-methoxyphenyl)acetate (5.00 g, 22 mmol) in toluene (30 mL) was added SOCl₂ (30 mL). The resulting mixture was stirred for 3 h at 100° C. and then cooled to room temperature. The mixture was concentrated under vacuum, and then dissolved in tetrahydrofuran (30 mL). To the solution was added a solution of trimethylsilyldiazomethane (18.4 mL, 2 M in hexane) and triethylamine (5.1 mL, 0.037 mol). The resulting solution was stirred for 10 h at room temperature. The reaction mixture was poured into saturated sodium bicarbonate solution (10 mL), and extracted with ethyl acetate (3×50 mL). The combined organic layers were dried over anhydrous sodium sulfate, filtered, and concentrated under vacuum. The residue was dissolved in methanol (200 mL) and then silver benzoate (3.00 g, 0.013 mol) and triethylamine (30 mL) were added. The resulting mixture was stirred for 10 h at room temperature and concentrated under vacuum. The material was purified by silica gel chromatography (eluting with 0:100 to 10:90 ethyl acetate/petroleum ether) to afford methyl 2-(3-bromo-4-methoxyphenyl) acetate as a yellow oil (2.00 g, 36%). LCMS (ES, m/z) 259, 261 [M+H]⁺.

### Step 3. methyl 2-(3-cyclopropyl-4-methoxyphenyl)acetate

To a solution of methyl 2-(3-bromo-4-methoxyphenyl) acetate (1.60 g, 0.78 mmol) in 1,4-dioxane (10 mL), water (1 ml) was added cyclopropylboronic acid (0.20 g, 1.20 mmol), potassium phosphate (986 mg, 2.30 mmol), and Pd(dppf)Cl₂ (56 mg, 0.039 mmol). The resulting mixture was stirred for 3 h at 90° C. and then cooled to room temperature. The reaction mixture was poured into water (10 mL) and extracted with ethyl acetate (3×20 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford methyl 2-(3-cyclopropyl-4-methoxyphenyl) acetate as a light yellow oil (840 mg, 62%). LCMS (ES, m/z) 221 [M+H]⁺.

### Intermediate 2-4. Methyl 2-(2-methyl-1,3-benzothiazol-4-yl)prop-2-enoate

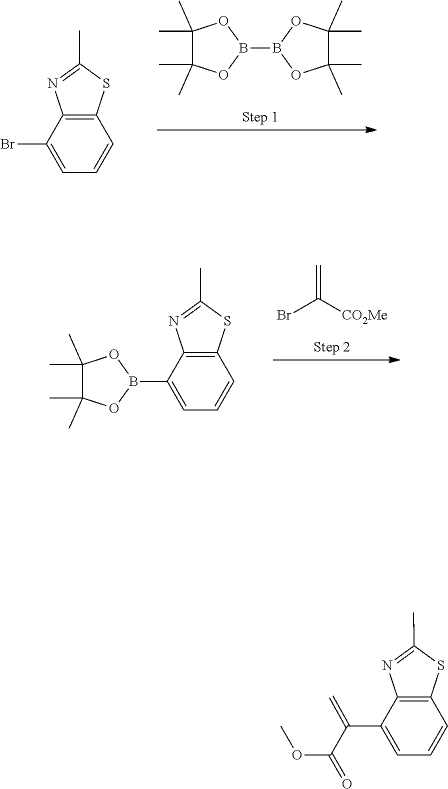

### Step 1. 2-Methyl-4-(4,4,5,5-tetramethyl-1,3,2-dioxaborolan-2-yl)-1,3-benzothiazole

To a solution of 4-bromo-2-methyl-1,3-benzothiazole (3.00 g, 12.9 mmol) in 1,4-dioxane (20 mL) was added 4,4,5,5-tetramethyl-2-(tetramethyl-1,3,2-dioxaborolan-2-yl)-1,3,2-dioxaborolane (4.01 g, 15.5 mmol), Pd(dppf)Cl₂ (960 mg, 1.29 mmol) and potassium acetate (2.58 g, 25.8 mmol). The resulting mixture was stirred for 16 h at 100° C. and then cooled to room temperature. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 2-methyl-4-(4,4,5,5-tetramethyl-1,3,2-dioxaborolan-2-yl)-1,3-benzothiazole as a light yellow oil (2.00 g, 46%). LCMS (ES, m/z) 276 [M+H]⁺.

### Step 2. Methyl 2-(2-methyl-1,3-benzothiazol-4-yl)prop-2-enoate

To a solution of 2-methyl-4-(4,4,5,5-tetramethyl-1,3,2-dioxaborolan-2-yl)-1,3-benzothiazole (600 mg, 1.86 mmol) in 1,4-dioxane (10 mL) was added methyl 2-bromoprop-2-enoate (447 mg, 2.66 mmol), XPhos 3G (80 mg, 0.11 mmol), potassium phosphate (1.4 g, 6.46 mmol) and water (1 mL). The resulting mixture was stirred for 16 h at 100° C. and then cooled to room temperature. The reaction mixture was poured into water (10 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford methyl 2-(2-methyl-1,3-benzothiazol-4-yl)prop-2-enoate as light yellow oil (280 mg, 55%). LCMS (ES, m/z) 234 [M+H]⁺.

The Intermediates in Table 2 were synthesized according to the procedure described for Intermediate 2-4 above.

TABLE 2

| Inter- | | LCMS: |
|---|---|---|
| me- | | (ESI) m/z |
| diate | Structure and Name | [M + H]⁺ |
| 2-6 | 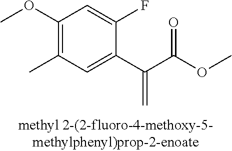 | 225 |
| 2-7 | 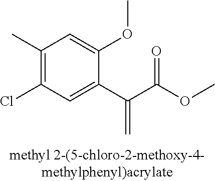 | 241 |
| 2-8 | 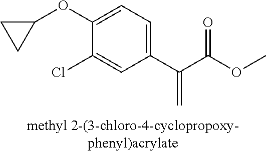 [SMILES: C=C(C(=O)OC)c1ccc(OC2CC2)c(Cl)c1] | 253 |
| 2-9 | 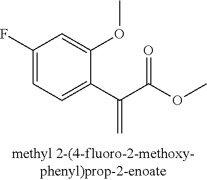 | 211 |
| 2-10 | 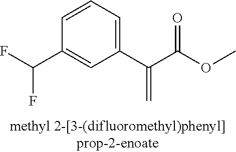 | 212 |
| 2-11 | 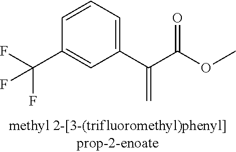 [SMILES: C=C(C(=O)OC)c1cccc(C(F)(F)F)c1] | 231 |
| 2-12 | 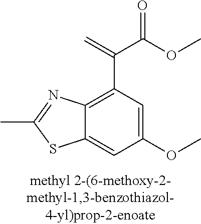 | 264 |
| 2-13A | 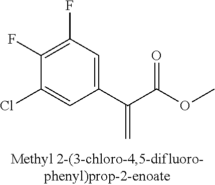 | 233 |
| 2-14 | 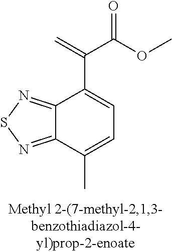 [SMILES: C=C(C(=O)OC)c1ccc(C)c2nsnc12] | 235 |
| 2-15 | 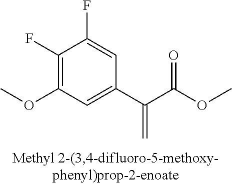 | 229 |
| 2-16 | 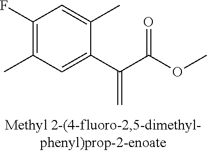 | 209 |
| 2-17 | 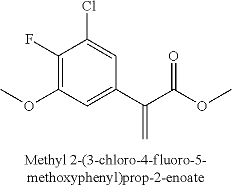 | 245 |
| 2-18 | 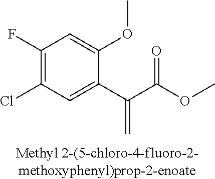 [SMILES: C=C(C(=O)OC)c1cc(Cl)c(F)cc1OC] | 245 |
| 2-19 | 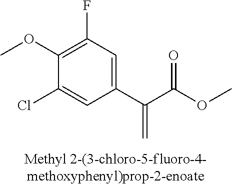 | 245 |
| 2-20 | 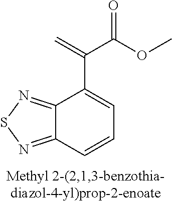 | 221 |
| 2-21 | 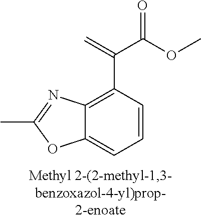 | 218 |
| 2-22 | 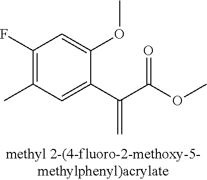 [SMILES: C=C(C(=O)OC)c1cc(C)c(F)cc1OC] | 225 |
| 2-23 | 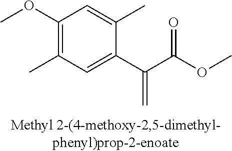 | 221 |
| 2-24 | 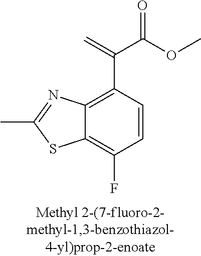 | 252 |
| 2-25 | 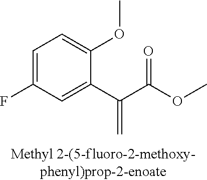 | 211 |
| 2-26 | 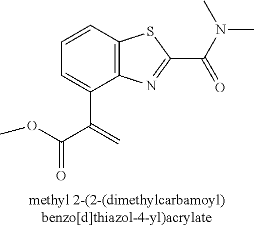 | 291 |
| 2-27 | 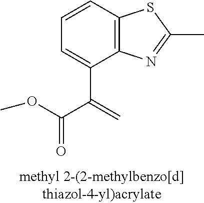 | 234 |

### Intermediate 3-1. Methyl 2-(3-methoxyphenyl)acrylate

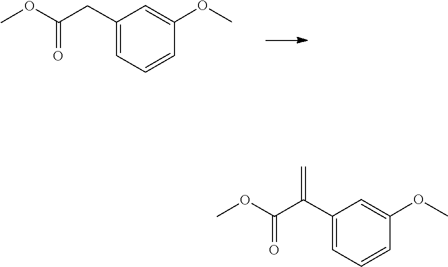

In a 250 mL round-bottom flask was placed methyl 2-(3-methoxyphenyl)acetate (5 g, 27.2 mmol), paraformaldehyde (3 g, 33.3 mmol), n-Bu₄NI (1 g, 2.7 mmol), potassium carbonate (9.6 g, 69.5 mmol) and N,N-dimethylformamide (60 mL). The resulting solution was stirred for 10 min at 60° C. in an oil bath. After cooling to room temperature, the solution was diluted with 100 mL of water and extracted with ethyl acetate (3×100 mL). The organic layers were combined, dried over anhydrous sodium sulfate, filtered and concentrated under vacuum to afford 3 g (570%) of methyl 2-(3-methoxyphenyl)prop-2-enoate as a yellow oil. MS: (ESI, m/z): 193[M+H]⁺.

The Intermediates in Table 3 were synthesized according to the procedure described for Intermediate 3-1 above.

TABLE 3

| Inter- | | LCMS: |
|---|---|---|
| me- | | (ESI) m/z |
| diate | Structure and Name | [M + H]⁺ |
| 3-2 | 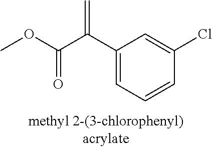 | 197 |
| 3-3 | 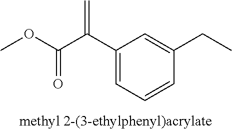 | 190 |
| 3-4 | 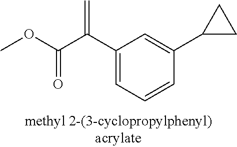 | 203 |
| 3-5 | 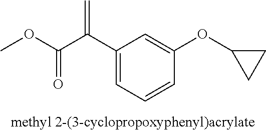 [SMILES: C=C(C(=O)OC)c1cccc(OC2CC2)c1] | 231 |
| 3-6 | 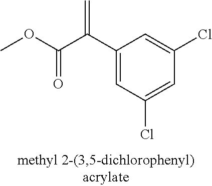 | 227 |
| 3-7 | 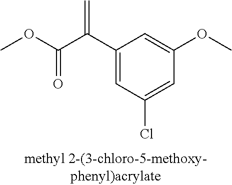 | 227 |
| 3-8 | 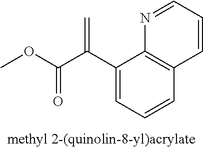 [SMILES: C=C(C(=O)OC)c1cccc2cccnc12] | 214 |
| 3-9 | 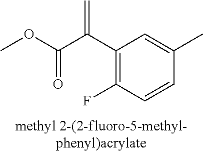 | 195 |
| 3-10 | 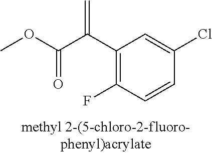 | 215 |
| 3-11 | 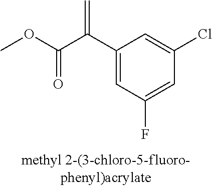 | 215 |
| 3-12 | 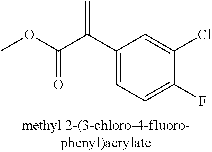 | 215 |
| 3-13 | 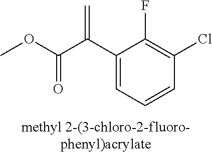 | 215 |
| 3-14 | 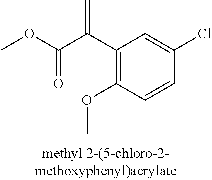 | 227 |
| 3-15 | 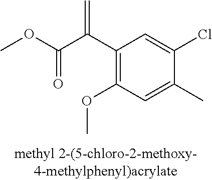 | 241 |
| 3-16 | 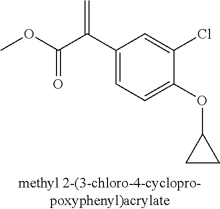 [SMILES: C=C(C(=O)OC)c1ccc(OC2CC2)c(Cl)c1] | 253 |
| 3-17 | 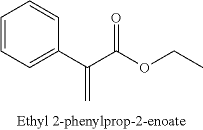 | 177 |

### Intermediate 4-1. (2S)-3-[[(tert-butoxy)carbonyl]amino]-2-(3-chlorophenyl)-2-fluoropropanoic Acid

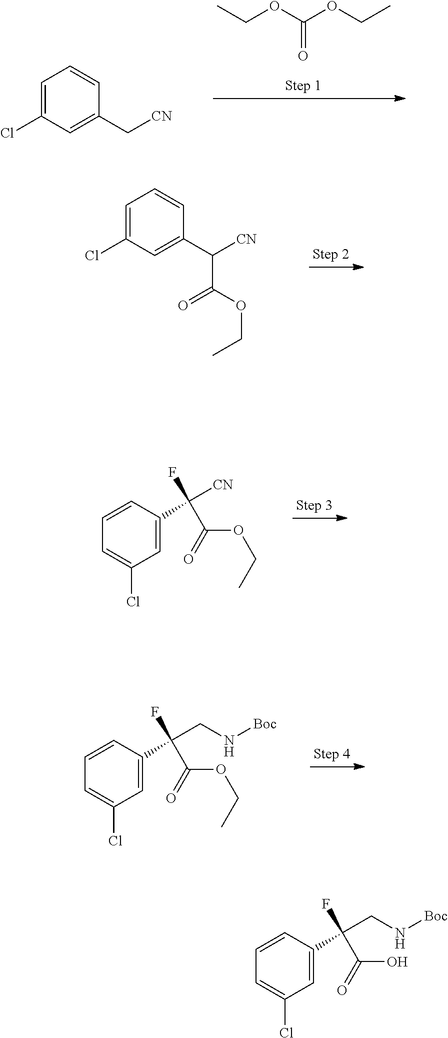

### Step 1. Ethyl 2-(3-chlorophenyl)-2-cyanoacetate

To a solution of 2-(3-chlorophenyl)acetonitrile (2.0 g, 12.5 mmol) in toluene (20 mL) was added sodium hydride (602 mg, 15.1 mmol, 60% dispersion in mineral oil) at 0° C. The resulting solution was stirred for 1 h at 0° C. followed by addition of diethyl carbonate (12.5 g, 100 mmol). The resulting mixture was stirred for 2 h at 80° C. and then cooled to room temperature. The reaction mixture was poured into aqueous ammonium chloride solution (20 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford ethyl 2-(3-chlorophenyl)-2-cyanoacetate as a yellow oil (1.50 g, 54%). LCMS (ES, m/z) 224, 226 [M+H]⁺.

### Step 2. Ethyl (2S)-2-(3-chlorophenyl)-2-cyano-2-fluoroacetate

To a solution of Selectfluor (1.90 g, 5.10 mmol) in MeCN (30 mL) was added (DHQD)₂PHAL (5.29 g, 6.80 mmol) and 3 Å molecular sieves (5 g). The resulting mixture was stirred for 1 h at room temperature and then cooled to −80° C. followed by slow addition of a solution of ethyl 2-(3-chlorophenyl)-2-cyanoacetate (800 mg, 3.40 mmol) in dichloromethane (40 mL) with stirring. After 6 h at −80° C., the reaction mixture was poured into water (20 mL) and then extracted with ethyl acetate (3×20 mL). The combined organic layers were dried over anhydrous sodium sulfate, filtered and concentrated under vacuum. The resulting crude product was purified by silica gel chromatography (eluting with 0:100 to 10:90 ethyl acetate/petroleum ether) to afford ethyl (2S)-2-(3-chlorophenyl)-2-cyano-2-fluoroacetate as a yellow oil (600 mg, 73%). LCMS (ES, m/z) 242, 244 [M+H]⁺.

### Step 3. ethyl (2S)-3-[[(tert-butoxy)carbonyl]amino]-2-(3-chlorophenyl)-2-fluoropropanoate

To a solution of ethyl (2S)-2-(3-chlorophenyl)-2-cyano-2-fluoroacetate (600 mg, 2.36 mmol) in ethanol (10 mL) was added Raney Ni (404 mg, 4.72 mmol) and Boc₂O (1.03 g, 4.72 mmol). Hydrogen was introduced with a balloon. The resulting mixture was stirred for 12 h at room temperature. The solids were filtered and the filtrate was concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (17% to 51% over 6.33 min); Flow rate: 60 mL/min; Detector: UV 254 nm). The fractions were concentrated to afford ethyl (2S)-3-[[(tert-butoxy)carbonyl]amino]-2-(3-chlorophenyl)-2-fluoropropanoate as colorless oil (200 mg, 25%). LCMS (ES, m/z) 290, 292 [M+H-t-Bu]⁺.

### Step 4. (2S)-3-[[(tert-butoxy)carbonyl]amino]-2-(3-chlorophenyl)-2-fluoropropanoic Acid

To a solution of ethyl (2S)-3-[[(tert-butoxy)carbonyl]amino]-2-(3-chlorophenyl)-2-fluoropropanoate (200 mg, 0.55 mmol) in tetrahydrofuran (2 mL) and water (2 mL) was added LiOH (66 mg, 2.76 mmol). The resulting mixture was stirred for 16 h at room temperature. The reaction mixture was washed with diethyl ether (1×10 mL) and then acidified to pH=5 with saturated aqueous citric acid. The product was extracted with ethyl acetate (3×20 mL). The combined organic layers were dried over anhydrous sodium sulfate, filtered, and concentrated under vacuum to afford (2S)-3-[[(tert-butoxy)carbonyl]amino]-2-(3-chlorophenyl)-2-fluoropropanoic acid as a yellow oil (120 mg, 69%). LCMS (ES, m/z) 262, 264 [M+H-t-Bu]⁺.

### Intermediate 5-1. 1-bromo-5-chloro-2-methoxy-4-methylbenzene

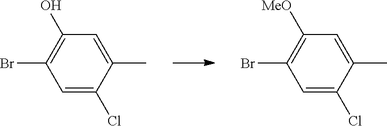

To a solution of 2-bromo-4-chloro-5-methylphenol (2.0 g, 8.1 mmol) in acetone (20 mL) was added potassium carbonate (2.5 g, 16 mmol), and iodomethane (0.66 mL, 9.5 mmol). The resulting mixture was stirred for 2 h at 25° C. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 1-bromo-5-chloro-2-methoxy-4-methylbenzene as a light yellow oil (1.87 g, 82%). GCMS (EI, m/z): 234, 236 [M]⁺.

The Intermediates in Table 4 were synthesized according to the procedure described for Intermediate 5-1 above.

TABLE 4

| | | LCMS: |
|---|---|---|
| | | (ESI) m/z |
| Intermediate | Structure and Name | [M + H]⁺ |
| 5-2 | 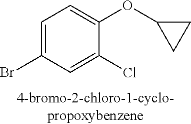 [SMILES: Clc1cc(Br)ccc1OC1CC1] | 247 |
| 5-3 | 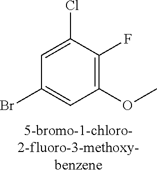 | 239 |
| 5-4 | 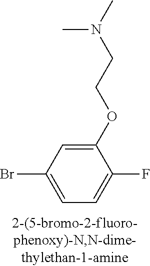 | |
| 5-5 | 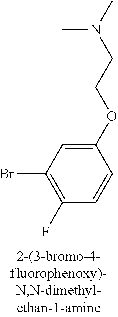 | |

### Intermediate 6-1. 2-{2-[(tert-butoxy)carbonyl]-7-fluoro-2,3-dihydro-1H-isoindol-4-yl}-2-oxo Acetic Acid

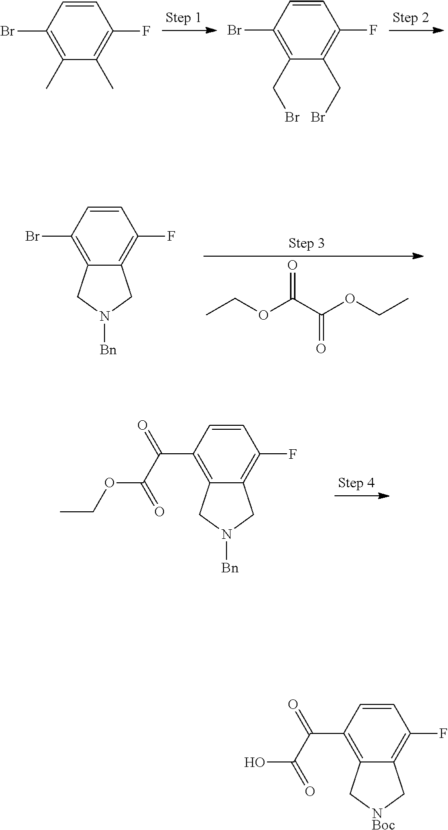

### Step 1. 1-bromo-2,3-bis(bromomethyl)-4-fluorobenzene

To a solution of 1-bromo-4-fluoro-2,3-dimethylbenzene (5.0 g, 23 mmol) in CCl₄ (75 mL), was added NBS (11 g, 59 mmol) and BPO (126 mg, 0.47 mmol). The resulting solution was stirred for 16 h at 85° C. and then cooled to room temperature. The reaction mixture was filtered and concentrated under vacuum. The resulting crude product was purified by silica gel chromatography (eluting with 1:100 ethyl acetate/petroleum ether) to afford 1-bromo-2,3-bis(bromomethyl)-4-fluorobenzene as a light yellow oil (7.0 g, 78%).

### Step 2. 2-benzyl-4-bromo-7-fluoro-2,3-dihydro-1H-isoindole

To a solution of 1-bromo-2,3-bis(bromomethyl)-4-fluorobenzene (3.0 g, 8.3 mmol) in MeCN (300 mL) was added 1-phenylmethanamine (900 mg, 8.3 mmol) and potassium bicarbonate (2.08 g, 20.8 mmol). The resulting mixture was stirred for 8 h at 85° C. and then cooled to room temperature. The reaction mixture was filtered and concentrated under vacuum. The crude product was purified by silica gel chromatography (eluting with 1:10 ethyl acetate/petroleum ether) to afford methyl 2-benzyl-4-bromo-7-fluoro-2,3-dihydro-1H-isoindole as a yellow oil (2 g, 78%). LCMS (ES, m/z) 306, 308 [M+H]⁺.

### Step 3. Ethyl 2-(2-benzyl-7-fluoro-2,3-dihydro-1H-isoindol-4-yl)-2-oxoacetate

To a solution of methyl 2-benzyl-4-bromo-7-fluoro-2,3-dihydro-1H-isoindole (3 g, 8.3 mmol) in THF (30 mL) was added a solution of n-butyllithium (12 mL, 1.6 M in THF) dropwise with stirring at −78° C. After stirring for 10 min, diethyl oxalate (8.5 mL, 81 mmol) was added. The solution was stirred for 1 h at −60° C. The reaction mixture was poured into saturate ammonium chloride solution (20 mL) and extracted with ethyl acetate (3×50 mL). The combined organic layers were dried over anhydrous sodium sulfate, filtered and concentrated under vacuum. The crude material was purified by silica gel chromatography (eluting with 1:5 ethyl acetate/petroleum ether) to afford ethyl 2-(2-benzyl-7-fluoro-2,3-dihydro-1H-isoindol-4-yl)-2-oxoacetate as a yellow oil (1.15 g, 36%). LCMS (ES, m/z) 328 [M+H]⁺.

### Step 4. 2-{2-[(tert-butoxy)carbonyl]-7-fluoro-2,3-dihydro-1H-isoindol-4-yl}-2-oxo Acetic Acid

To a solution of 2-(2-benzyl-7-fluoro-2,3-dihydro-1H-isoindol-4-yl)-2-oxo acetate (1 g, 3.0 mmol) in 1,2-dichloroethane (10 mL) was added 1-chloroethyl carbonochloridate (396 mg, 2.8 mmol). The resulting solution was stirred for 2 h at 85° C. and cooled to room temperature. The solution was concentrated under vacuum and dissolved in MeOH (10 mL). After stirring for 1 h at 85° C., the reaction was cooled to room temperature. The resulting mixture was concentrated and dissolved in water (10 mL). The resulting solution was washed with EtOAc (1×10 mL). The aqueous layer was rendered basic (pH=10) with sodium hydroxide solution (1 N). To this solution was added Boc₂O (642 mg, 2.8 mmol). The resulting solution was stirred for 4 h at rt and then extracted with ethyl acetate (2×10 mL). The combined organic layers were dried over anhydrous sodium sulfate, filtered and concentrated under vacuum. The crude product was purified by silica gel chromatography (eluting with 2:1 ethyl acetate/petroleum ether) to afford 2-{2-[(tert-butoxy)carbonyl]-7-fluoro-2,3-dihydro-1H-isoindol-4-yl}-2-oxoacetic acid as a yellow solid (300 mg, 32%). LCMS (ES, m/z) 310 [M+H]⁺.

### Intermediate 7-1. 2-{2-[(tert-butoxy)carbonyl]-2,3-dihydro-1H-isoindol-4-yl}-2-oxoacetic Acid

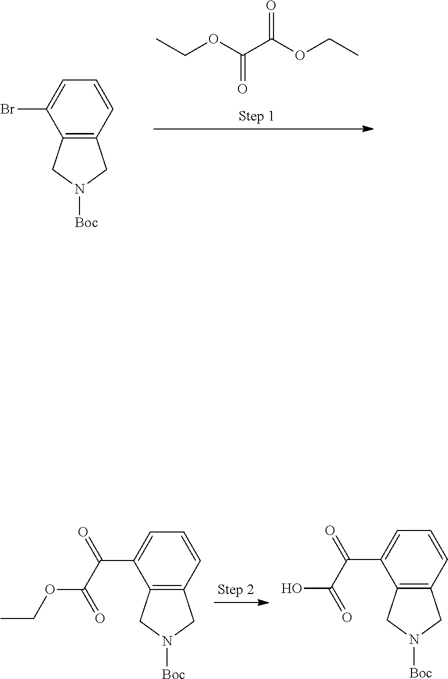

### Step 1. tert-butyl 4-(2-ethoxy-2-oxoacetyl)-2,3-dihydro-1H-isoindole-2-carboxylate

To a solution of tert-buty 4-bromo-2,3-dihydro-1H-isoindole-2-carboxylate in (2.0 g, 6.4 mmol) in THF (20 mL) was added a solution of n-BuLi (2.6 mL, 2.5 M in THF) dropwise with stirring at −78° C. After stirring for 15 min at −78° C., diethyl oxalate (3.1 mL, 32 mmol) was added in. The resulting mixture was stirred for 1 h at −60° C. The reaction mixture was poured into saturated ammonium chloride solution (20 mL) and then extracted with ethyl acetate (3×20 mL). The combined organic layers were dried over anhydrous sodium sulfate, filtered and concentrated under vacuum. The resulting crude product was purified by silica gel chromatography (eluting with 0:100 to 20:80 ethyl acetate/petroleum ether) to afford tert-butyl 4-(2-ethoxy-2-oxoacetyl)-2,3-dihydro-1H-isoindole-2-carboxylate as a light yellow solid (1.12 g, 47%). LCMS (ES, m/z) 320 [M+H]⁺.

### Step 2. 2-{2-[(tert-butoxy)carbonyl]-2,3-dihydro-1H-isoindol-4-yl}-2-oxoacetic Acid

To a solution of tert-butyl 4-(2-ethoxy-2-oxoacetyl)-2,3-dihydro-1H-isoindole-2-carboxylate (1.12 g, 2.98 mmol) in tetrahydrofuran (6 mL) was added water (6 mL) and LiOH (421 mg, 16.70 mmol). The resulting mixture was stirred for 16 h at room temperature. The reaction mixture was washed with diethyl ether (1×10 mL) and then acidified to pH=5 with saturated citric acid. The resulting solution was extracted with ethyl acetate (2×10 mL). The combined organic layers were combined, dried over anhydrous sodium sulfate, filtered and concentrated under vacuum to afford 2-[2-[(tert-butoxy)carbonyl]-2,3-dihydro-1H-isoindol-4-yl]-2-oxoacetic acid as a light yellow solid (1.0 g, crude). LCMS (ES, m/z) 292 [M+H]⁺.

The Intermediates in Table 5 were synthesized according to the procedure described for Intermediate 7-1 above.

TABLE 5

| Inter- | | LCMS: |
|---|---|---|
| me- | | (ESI) m/z |
| diate | Structure and Name | [M + H]⁺ |
| 7-2 | 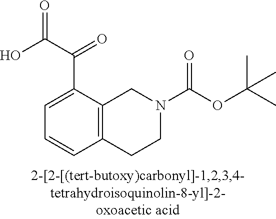 [SMILES: CC(C)(C)OC(=O)N1CCc2cccc(C(=O)C(=O)O)c2C1] | 306 |
| 7-3 | 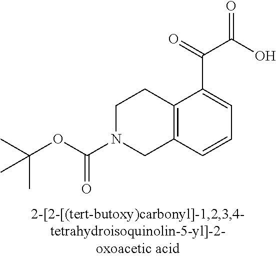 [SMILES: CC(C)(C)OC(=O)N1CCc2c(cccc2C(=O)C(=O)O)C1] | 306 |
| 7-4 | 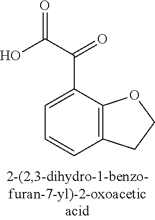 [SMILES: O=C(O)C(=O)c1cccc2c1OCC2] | 193 |
| 7-5 | 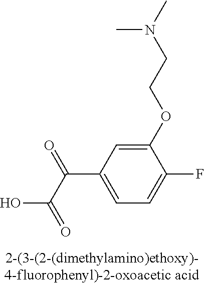 [SMILES: CN(C)CCOc1cc(C(=O)C(=O)O)ccc1F] | 256 |
| 7-6 | 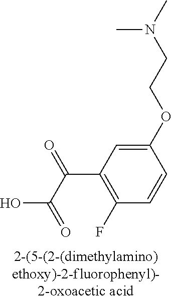 [SMILES: CN(C)CCOc1ccc(F)c(C(=O)C(=O)O)c1] | 256 |
| 7-7 | 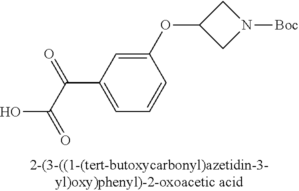 [SMILES: CC(C)(C)OC(=O)N1CC(Oc2cccc(C(=O)C(=O)O)c2)C1] | 322 |

### Intermediate 8-1. Methyl 2-(3-cyclopropoxyphenyl) acetate

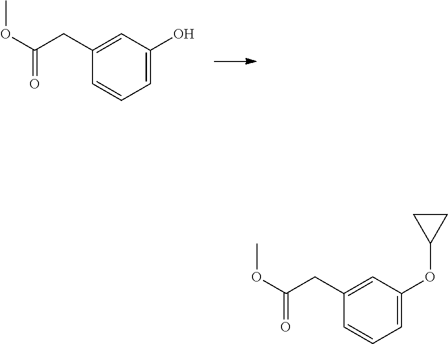

Into a 200 mL sealed tube purged and maintained with an inert atmosphere of nitrogen was added methyl 2-(3-hydroxyphenyl)acetate (5 g, 30.1 mmol), bromocyclopropane (20 g, 151 mmol), DMA (100 ml) and Cs₂CO₃ (30 g, 90.4 mmol). The resulting mixture was stirred for 48 h at 150° C. in an oil bath. After cooling to room temperature, the reaction mixture was treated with 100 mL of water and then extracted with ethyl acetate (3×100 mL). The organic layers were combined, dried over anhydrous sodium sulfate, filtered and concentrated under vacuum. The residue was purified by silica gel chromatography (eluting with 1:10 ethyl acetate/petroleum ether) to afford 1.5 g (22%) of methyl 2-(3-cyclopropoxyphenyl) acetate as a red oil. MS: (EI, m/z): 206[M]⁺.

### Intermediate 9-1. Ethyl 2-(quinolin-8-yl) acetate

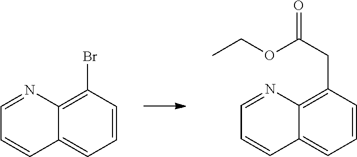 [SMILES: Brc1cccc2cccnc12 ; CCOC(=O)Cc1cccc2cccnc12]

Into a 250 mL 3-necked round-bottom flask purged and maintained with an inert atmosphere of nitrogen was added 8-bromoquinoline (5 g, 24.0 mmol), potassium 3-ethoxy-3-oxopropanoate (8.2 g, 48.2 mmol), BINAP (1.8 g, 2.9 mmol), 4-dimethylaminopyridine (295 mg, 2.4 mmol), Pd₂(allyl)₂Cl₂ (352 mg, 0.96 mmol) and mesitylene (60 mL). The resulting mixture was stirred for 7 h at 140° C. After cooling to room temperature, the reaction mixture was treated with water (60 mL) and then extracted with ethyl acetate (3×100 mL). The organic layers were combined, washed with brine (80 mL), dried over anhydrous sodium sulfate, filtered and concentrated under vacuum. The residue was purified by silica gel chromatography (eluting with 7/3 ethyl acetate/petroleum ether) to afford 3 g (58%) of ethyl 2-(quinolin-8-yl) acetate as a brown oil. MS: (ESI, m/z): 216[M+H]⁺.

### Intermediate 10-1. tert-Butyl 3-(5-(2,3-dihydrobenzo[b][1,4]dioxin-6-ylsulfonyl)-4,5-dihydropyrrolo[3,4-c]pyrrol-2(1H,3H,4H)-yl)-2-(3-ethylphenyl)-3-oxopropyl(methyl)carbamate

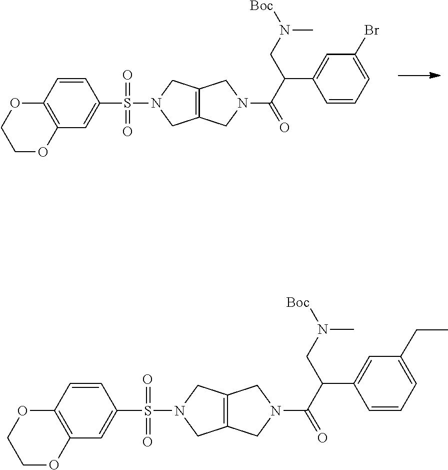 [SMILES: CN(CC(C(=O)N1CC2=C(C1)CN(S(=O)(=O)c1ccc3c(c1)OCCO3)C2)c1cccc(Br)c1)C(=O)OC(C)(C)C ; CCc1cccc(C(CN(C)C(=O)OC(C)(C)C)C(=O)N2CC3=C(C2)CN(S(=O)(=O)c2ccc4c(c2)OCCO4)C3)c1]

Into an 8 mL vial purged and maintained with an inert atmosphere of nitrogen was added tert-butyl 2-(3-bromophenyl)-3-(5-(2,3-dihydrobenzo[b][1,4]dioxin-6-ylsulfonyl)-4,5-dihydropyrrolo[3,4-c]pyrrol-2(1H,3H,4H)-yl)-3-oxopropyl(methyl)carbamate (120 mg, 0.18 mmol), Pd(dppf)Cl₂.CH₂Cl₂ (15 mg, 0.02 mmol), potassium carbonate (51 mg, 0.37 mmol), THF (1 mL) and a solution of diethylzinc in THF (1.0 M, 0.3 mL, 0.3 mmol). The resulting solution was stirred overnight at 20° C. The resulting solution was poured into 10 mL of water and then extracted with ethyl acetate (3×10 mL). The organic layers were combined, dried over anhydrous sodium sulfate, filtered and concentrated under vacuum. The residue was purified by silica gel chromatography (eluting with 1:1 ethyl acetate/petroleum ether) to afford 40 mg (38%) of tert-butyl 3-(5-(2,3-dihydrobenzo[b][1,4]dioxin-6-ylsulfonyl)-4,5-dihydropyrrolo[3,4-c]pyrrol-2(1H,3H,4H)-yl)-2-(3-ethylphenyl)-3-oxopropyl(methyl)carbamate as a light yellow oil. MS: (ESI, m/z): 598 [M+H]⁺.

### Intermediate 11-1. Methyl 2-(3,5-dichlorophenyl)acetate

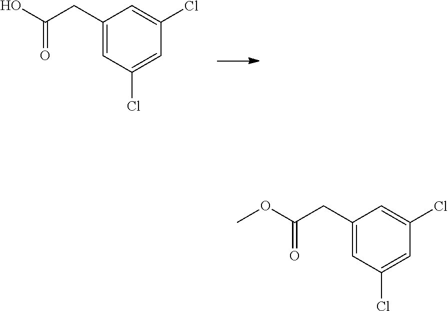

Into a 100 mL round-bottom flask was added 2-(3,5-dichlorophenyl)acetic acid (3 g, 14.6 mmol) and thionyl chloride (30 mL). The resulting solution was stirred for 1 h at 40° C. The reaction mixture was concentrated under vacuum and dissolved in 20 mL of methanol. The resulting solution was stirred for 10 min at 60° C. After cooling to room temperature, the reaction mixture was concentrated, treated with 10 mL of saturated sodium bicarbonate solution and then extracted with ethyl acetate (3×30 mL). The organic layers were combined, dried over anhydrous sodium sulfate, filtered and concentrated under vacuum to afford 2.6 g (73%) of methyl 2-(3,5-dichlorophenyl)acetate as a colorless oil. MS: (EI, m/z): 218, 220[M]⁺.

### Intermediate 12-1. tert-Butyl 2-(3-cyclopropylphenyl)-3-(5-(2,3-dihydrobenzo[b][1,4]dioxin-6-ylsulfonyl)-4,5-dihydropyrrolo[3,4-c]pyrrol-2(1H,3H,4H)-yl)-3-oxopropyl(methyl)carbamate

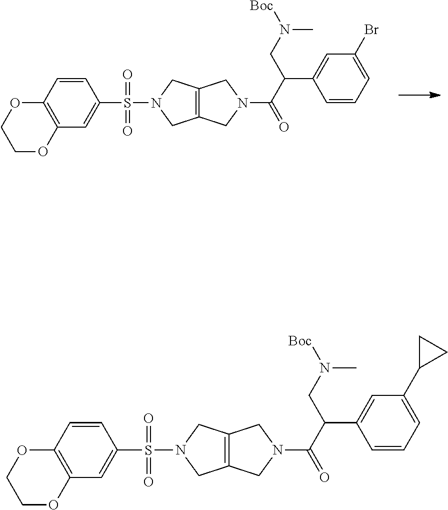 [SMILES: CN(CC(C(=O)N1CC2=C(C1)CN(S(=O)(=O)c1ccc3c(c1)OCCO3)C2)c1cccc(Br)c1)C(=O)OC(C)(C)C ; CN(CC(C(=O)N1CC2=C(C1)CN(S(=O)(=O)c1ccc3c(c1)OCCO3)C2)c1cccc(C2CC2)c1)C(=O)OC(C)(C)C]

In a 8 mL vial purged and maintained with an inert atmosphere of nitrogen was added tert-butyl 2-(3-bromophenyl)-3-(5-(2,3-dihydrobenzo[b][1,4]dioxin-6-ylsulfonyl)-4,5-dihydropyrrolo[3,4-c]pyrrol-2(1H,3H,4H)-yl)-3-oxopropyl(methyl)carbamate (120 mg, 0.18 mmol), cyclopropylboronic acid (19 mg, 0.22 mmol), Cs2CO3 (121 mg, 0.37 mmol), 3rd Generation XPhos precatalyst (16 mg, 0.02 mmol), 1,4-dioxane (0.9 mL) and water (0.3 mL). The resulting mixture was stirred for 3 h at 80° C. in an oil bath. After cooling to room temperature, the resulting mixture was concentrated under vacuum. The residue was purified by silica gel chromatography (eluting with 1:1 ethyl acetate/petroleum ether) to afford 80 mg (75%) of tert-butyl 2-(3-cyclopropylphenyl)-3-(5-(2,3-dihydrobenzo[b][1,4]dioxin-6-ylsulfonyl)-4,5-dihydropyrrolo[3,4-c]pyrrol-2(1H,3H,4H)-yl)-3-oxopropyl(methyl)carbamate as a yellow oil. MS: (ESI, m/z): 610[M+H]⁺.

### Intermediate 13-1. Methyl 2-(3-chloro-5-methoxyphenyl) acrylate

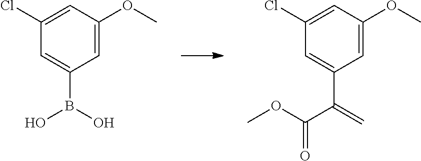

In a 50 mL round-bottom flask purged and maintained with an inert atmosphere of nitrogen was added (3-chloro-5-methoxyphenyl)boronic acid (500 mg, 2.6 mmol), methyl 2-bromoprop-2-enoate (440 mg, 2.6 mmol), 1,4-dioxane (0.9 mL), water (0.3 mL), Cs2CO3 (1.75 g, 5.37 mmol) and 3rd Generation XPhos precatalyst (225 mg, 0.27 mmol). The resulting mixture was stirred for 3 h at 100° C. in an oil bath. After cooling to room temperature, the reaction mixture was concentrated under vacuum. The residue was purified by silica gel chromatography (eluting with 1:5 ethyl acetate/petroleum ether) to afford 230 mg (39%) of methyl 2-(3-chloro-5-methoxyphenyl) acrylate as a yellow oil. MS: (ESI, m/z): 227[M+H]+.

The Intermediates in Table 6 were synthesized according to the procedure described for Intermediate 13-1 above.

TABLE 6

| | | LCMS: (ESI) |
|---|---|---|
| Intermediate | Structure and Name | m/z [M + H]⁺ |
| 13-2 | 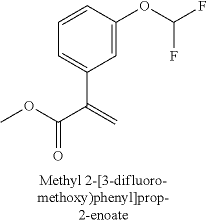 [SMILES: C=C(C(=O)OC)c1cccc(OC(F)F)c1] | 229 |
| 13-3 | 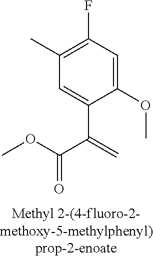 | 225 |
| 13-4 | 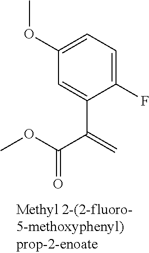 | 211 |

### Intermediate 14-1. methyl 3-{[(tert-butoxy)carbonyl](methyl)amino}-2-(3-chloro-4-fluoro-2-methoxyphenyl)propanoate

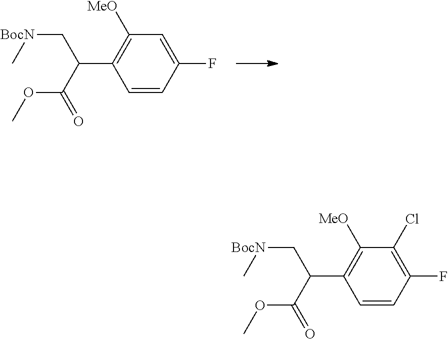 [SMILES: COC(=O)C(CN(C)C(=O)OC(C)(C)C)c1ccc(F)cc1OC ; COC(=O)C(CN(C)C(=O)OC(C)(C)C)c1ccc(F)c(Cl)c1OC]

To a solution of methyl 3-{[(tert-butoxy)carbonyl](methyl)amino}-2-(4-fluoro-2-methoxyphenyl)propanoate (180 mg, 0.53 mmol) in dichloromethane (3 mL) was added NCS (211 mg, 1.58 mmol). The resulting solution was stirred for 16 h at room temperature. The reaction mixture were filtered, and concentrated under vacuum. The resulting crude product was purified by prep-TLC (eluting with 1:5 ethyl acetate/petroleum ether) to afford methyl 3-{[(tert-butoxy)carbonyl](methyl)amino}-2-(3-chloro-4-fluoro-2-methoxyphenyl)propanoate as a light yellow oil (160 mg, 81%). LCMS (ES, m/z) 376, 378 [M+H]⁺.

### Intermediate 15-1. Tert-butyl N-(methoxymethyl)-N-methylcarbamate

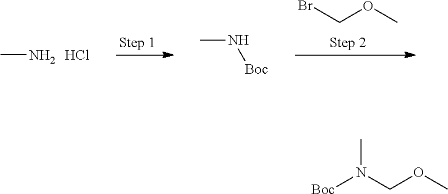

To a solution of tert-butyl N-methylcarbamate (10 g, 0.72 mol) in tetrahydrofuran (200 mL) was added a solution of NaHMDS (44 mL, 2 M in THF) slowly with stirring at 0° C. After stirring for 30 min at this temperature, this was followed by addition of a solution of bromo(methoxy)methane (11.4 g, 0.91 mol) in tetrahydrofuran (10 mL) dropwise with stirring at 0° C. The resulting solution was allowed to warm to room temperature naturally and stirred for 16 h. The reaction mixture was treated with saturated ammonium chloride solution (200 mL) and then extracted with ethyl acetate (3×200 mL). The combined organic layers were dried over anhydrous sodium sulfate, filtered, and concentrated under vacuum. The resulting crude product was purified by silica gel chromatography (eluting with 0:100 to 5:95 ethyl acetate/petroleum ether) to afford tert-butyl N-(methoxymethyl)-N-methylcarbamate as a yellow oil (12.5 g, 89%). ¹H-NMR (CDCl₃, 400 MHz) δ (ppm): 4.65 (s, 2H), 3.25 (s, 3H), 2.87 (s, 3H), 1.46 (s, 9H). LCMS (ES, m/z) 176 [M+H]⁺.

### Intermediate 16-1. (2R)-3-{[(tert-butoxy)carbonyl](methyl)amino}-2-(3-chlorophenyl)propanoic Acid

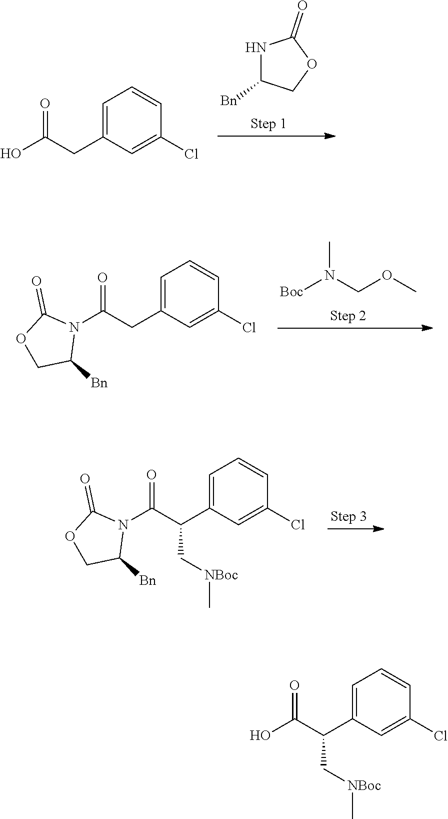 [SMILES: O=C(O)Cc1cccc(Cl)c1 ; O=C1N[C@@H](Cc2ccccc2)CO1 ; O=C(Cc1cccc(Cl)c1)N1C(=O)OC[C@@H]1Cc1ccccc1 ; COCN(C)C(=O)OC(C)(C)C ; CN(C[C@H](C(=O)N1C(=O)OC[C@@H]1Cc1ccccc1)c1cccc(Cl)c1)C(=O)OC(C)(C)C ; CN(C[C@H](C(=O)O)c1cccc(Cl)c1)C(=O)OC(C)(C)C]

### Step 1. (4S)-4-benzyl-3-[2-(3-chlorophenyl)acetyl]-1,3-Oxazolidin-2-one

To a solution of 2-(3-chlorophenyl)acetic acid (23.2 g, 0.14 mol) in toluene (300 mL) was added (4S)-4-benzyl-1,3-oxazolidin-2-one (20 g, 0.11 mol) followed by the slow addition of TEA (46 g, 0.45 mol) with stirring at 15° C., and then slow addition of 2,2-dimethylpropanoyl chloride (17.4 g, 0.14 mol) with stirring at 30° C. The resulting mixture was stirred for 3 h at 110° C., and then cooled to room temperature. The mixture was concentrated under vacuum and the resulting crude product was purified by silica gel chromatography (eluting with 0:100 to 5:95 ethyl acetate/petroleum ether) to afford (4S)-4-benzyl-3-[2-(3-chlorophenyl)acetyl]-1,3-Oxazolidin-2-one as a yellow solid (18 g, 40%). ¹H-NMR (CDCl₃, 400 MHz) δ (ppm): 7.43-7.12 (m, 9H), 4.76-4.64 (m, 1H), 4.41-4.15 (m, 4H), 3.35-3.23 (m, 1H), 2.87-2.74 (m, 1H). LCMS (ES, m/z) 330, 332 [M+H]⁺.

### Step 2. tert-butyl N-[(2R)-3-[(4S)-4-benzyl-2-oxo-1,3-oxazolidin-3-yl]-2-(3-chlorophenyl)-3-oxopropyl]-N-methylcarbamate

To a solution of (4S)-4-benzyl-3-[2-(3-chlorophenyl)acetyl]-1,3-oxazolidin-2-one (15 g, 0.41 mol) in dichloromethane (180 mL) was added a solution of titanium(IV) chloride (48.4 mL, 1 M in DCM) dropwise with stirring at −20° C. After stirring for 2 h at −20° C., a solution of DIEA (5.1 mL, 0.31 mol) in DCM (10 mL) was added slowly with stirring. After 1.5 h at −20° C., tert-butyl N-(methoxymethyl)-N-methylcarbamate (10.4 g, 0.59 mol) in dichloromethane (10 mL) was added dropwise. The reaction mixture was stirred for 2 h at −20° C. and then treated with saturate ammonium chloride solution (100 mL). The product was extracted with ethyl acetate (3×300 mL). The combined organic layers were dried over anhydrous sodium sulfate, filtered, and concentrated under vacuum to afford tert-butyl N-[(2R)-3-[(4S)-4-benzyl-2-oxo-1,3-oxazolidin-3-yl]-2-(3-chlorophenyl)-3-oxopropyl]-N-methylcarbamate as a yellow solid (18 g, 84%).

### Step 3. (2R)-3-{[(tert-butoxy)carbonyl](methyl)amino}-2-(3-chlorophenyl)propanoic Acid

To a solution of lithium hydroxide (2.3 g, 0.09 mol) in water (125 mL) was added THF (170 mL) followed by the sequential addition of a solution of hydrogen peroxide (9.2 mL, 30% in water) and a solution of tert-butyl N-[(2R)-3-[(4S)-4-benzyl-2-oxo-1,3-oxazolidin-3-yl]-2-(3-chlorophenyl)-3-oxopropyl]-N-methylcarbamate (18 g, 0.04 mol) in tetrahydrofuran (10 mL) dropwise with stirring at 0° C. The resulting mixture was stirred for 3 h at 0° C. The reaction was carefully quenched with aqueous sodium sulfite solution (100 mL, 12.5 wt %) while maintaining reaction temperature <10° C. After stirring for 30 min at room temperature, the mixture (pH=14) was concentrated to remove organic solvent and then washed with diethyl ether (3×200 mL). The aqueous layer was then acidified to pH=2-3 with aqueous potassium bisulfate solution (27 wt %) while maintaining temperature <15° C. The resulting solution was extracted with ethyl acetate (3×300 mL). The combined organic layers were dried over anhydrous sodium sulfate, filtered and concentrated under vacuum to afford (2R)-3-{[(tert-butoxy)carbonyl](methyl)amino}-2-(3-chlorophenyl)propanoic acid as a yellow oil (10 g, 84%). Note: the material contains about 20 wt % of (4S)-4-benzyl-1,3-oxazolidin-2-one based on HNMR determination, and its ee value is about 96%.

The crude material (4.5 g) was dissolved in MeCN (5 mL) and N-cyclohexylcyclohexanamine (3 g, 16.5 mmol) was added. The reaction was heated to 60° C. for 3 h and cooled to room temperature slowly over 16 h without stirring. The solids were collected by filtration and dried under vacuum to afford N-cyclohexylcyclohexanamine (2R)-3-{[(tert-butoxy)carbonyl](methyl)amino}-2-(3-chlorophenyl) propanoic acid complex as a white solid (5 g). The complex was then dissolved with aqueous solution of KHSO₄ (50 mL, 27 wt %) and EtOAc (50 mL). The resulting solution was stirred for 1.5 h at rt and extracted with EtOAc (3×50 mL). The combined organic layers were dried over anhydrous sodium sulfate, filtered and concentrated under vacuum to afford pure (2R)-3-{[(tert-butoxy)carbonyl](methyl)amino}-2-(3-chlorophenyl)propanoic acid as a white solid (2.30 g, 99% purity, >99% ee). ¹H-NMR (DMSO-d₆, 400 MHz) δ (ppm): 7.43-7.21 (m, 4H), 4.07-3.90 (m, 1H), 3.94-3.89 (m, 2H), 2.84-2.70 (m, 3H), 1.39 (s, 9H). LCMS (ES, m/z) 314, 316 [M+H]⁺.

### Intermediate 17-1. 1-Bromo-4-fluoro-2-methoxy-5-methylbenzene

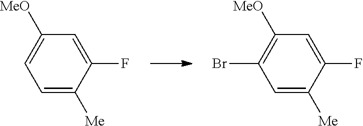 [SMILES: COc1ccc(C)c(F)c1 ; COc1cc(F)c(C)cc1Br]

To a solution of 2-fluoro-4-methoxy-1-methylbenzene (8.00 g, 57.1 mmol) in MeCN (60 mL) was added NBS (11.2 g, 62.8 mmol). The resulting mixture was stirred for 3 h at room temperature. The reaction mixture was poured into water (40 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 1-bromo-4-fluoro-2-methoxy-5-methylbenzene as colorless oil (5.2 g, 35%). LCMS (ES, m/z): 219, 221 [M+H]⁺.

The Intermediates in Table 7 were synthesized according to the procedure described for Intermediate 17-1 above.

TABLE 7

| | | LCMS: (ESI) |
|---|---|---|
| Intermediate | Structure and Name | m/z [M + H]⁺ |
| 17-2 | 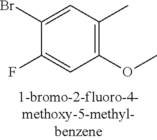 | 219 |

### Intermediate 18-1. 4-fluoro-2-methoxy-5-methylphenyl)boronic Acid

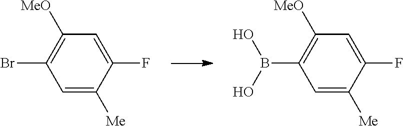 [SMILES: COc1cc(F)c(C)cc1Br ; COc1cc(F)c(C)cc1B(O)O]

To a solution of 1-bromo-4-fluoro-2-methoxy-5-methylbenzene (5.20 g, 23.7 mmol) in THF (25 mL) was added tris(propan-2-yl) borate (7.74 mL, 33.5 mmol). Then n-BuLi (11 mL, 2.50 M in THF) was added at −78° C. dropwise. The resulting mixture was stirred for 2 h at −30° C. and then was allowed to warm to room temperature. The reaction was quenched by addition of sulfuric acid (25 mL, 0.2 M) and extracted with ethyl acetate (3×60 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford (4-fluoro-2-methoxy-5-methylphenyl)boronic acid as a white solid (2.20 g, 50%). LCMS (ES, m/z): 185 [M+H]⁺.

### Intermediate 19-1. Tert-butyl 5-bromo-1,2,3,4-tetrahydroisoquinoline-2-carboxylate

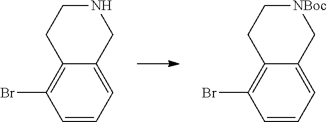 [SMILES: Brc1cccc2c1CCNC2 ; CC(C)(C)OC(=O)N1CCc2c(Br)cccc2C1]

To a solution of 5-bromo-1,2,3,4-tetrahydroisoquinoline (3.00 g, 14.1 mmol) in dichloromethane (30 mL) was added TEA (6.17 mL, 44.5 mmol), di-tert-butyl dicarbonate (9.30 g, 42.6 mmol). The solution was stirred for 16 h at room temperature and concentrated under vacuum. The crude product was purified by silica gel chromatography (eluting with 1:5 ethyl acetate/petroleum ether) to afford tert-butyl 5-bromo-1,2,3,4-tetrahydroisoquinoline-2-carboxylate as a white solid (2.70 g, 61%). LCMS (ES, m/z): 312, 314 [M+H]⁺.

### Intermediate 20-1. 3-{[(tert-butoxy)carbonyl]amino}-2-(2-methylcyclopentyl)propanoic Acid

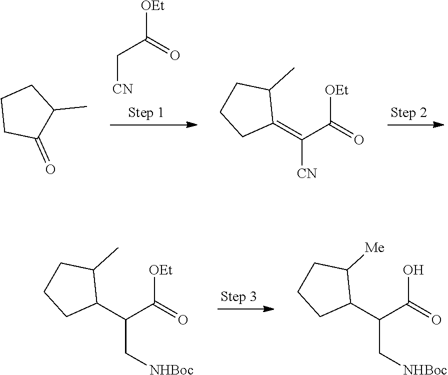

### Step 1. Ethyl 2-cyano-2-[(1Z)-2-methylcyclopentylidene]acetate

To a solution of 2-methylcyclopentan-1-one (5.00 g, 50.9 mmol) in toluene (100 mL) was added ammonium acetate (2.70 g, 35.7 mmol), acetic acid (2.8 mL), and ethyl 2-cyanoacetate (5.70 g, 50.9 mmol). The resulting mixture was stirred for 24 h at 100° C. and then cooled to room temperature. The solution was poured into water (100 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford ethyl 2-cyano-2-[(1Z)-2-methylcyclopentylidene]acetate as colorless oil (2.50 g, 25%). LCMS (ES, m/z): 194 [M+H]⁺.

### Step 2. Ethyl 3-{[(tert-butoxy)carbonyl]amino}-2-(2-methylcyclopentyl)propanoate

To a solution of ethyl 2-cyano-2-[(1Z)-2-methylcyclopentylidene]acetate (1.30 g, 6.73 mmol) in ethanol (100 mL) was added palladium carbon (200 mg, 10 wt % Pd). Hydrogen was introduced with a balloon. The resulting mixture was stirred for 16 h at room temperature. The reaction mixture was filtered and then treated with Raney Ni (400 mg), triethylamine (2.74 mL, 20.2 mmol) and di-tert-butyl dicarbonate (4.40 g, 20.2 mmol). Hydrogen was introduced with a balloon. The solution was stirred for another 16 h at room temperature. The reaction mixture was filtered and concentrated under vacuum. The crude product was purified by silica gel chromatography (eluting with 0:100 to 10:90 ethyl acetate/petroleum ether) to afford ethyl 3-[[(tert-butoxy)carbonyl]amino]-2-(2-methylcyclopentyl)propanoate as a colorless oil (1.00 g, 50%). LCMS (ES, m/z): 300 [M+H]⁺.

### Step 3. 3-{[(tert-butoxy)carbonyl]amino}-2-(2-methylcyclopentyl)propanoic Acid

To a solution of ethyl 3-[[(tert-butoxy)carbonyl]amino]-2-(2-methylcyclopentyl)propanoate (500 mg, 1.67 mmol) in tetrahydrofuran (8 mL) and water (8 mL) was added lithium hydroxide (200 mg, 8.35 mmol). The resulting mixture was stirred for 14 h at 45° C. The mixture was diluted with water (20 mL) and washed with ethyl acetate (15 mL). The pH value of the aqueous solution was adjusted to 5-6 with saturated aqueous citric acid. The product was extracted with ethyl acetate (3×15 mL). The combined organic layers were dried over anhydrous sodium sulfate, filtered, and concentrated under vacuum to afford 3-[[(tert-butoxy)carbonyl]amino]-2-(2-methylcyclopentyl)propanoic acid as a colorless oil (350 mg, 77%). LCMS (ES, m/z): 272 [M+H]⁺.

### Intermediate 21-1. Tert-butyl 4-formyl-2,3-dihydro-1H-isoindole-2-carboxylate

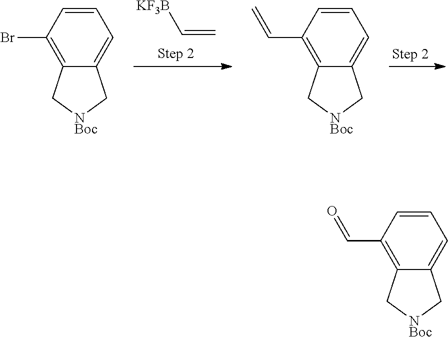

### Step 1. Tert-butyl 4-ethenyl-2,3-dihydro-1H-isoindole-2-carboxylate

To a solution of tert-butyl 4-bromo-2,3-dihydro-1H-isoindole-2-carboxylate (6.10 g, 20.5 mmol) in 1,4-dioxane (60 mL) was added potassium ethenyltrifluoroborate (4.34 g, 40.9 mmol), Pd(dppf)Cl₂ (1.50 g, 2.05 mmol), and a solution of sodium carbonate (4.10 g, 30.6 mmol) in water (20 mL). The resulting mixture was stirred for 16 h at 100° C. and then cooled to room temperature. The reaction mixture was poured into water (30 mL) and extracted with ethyl acetate (3×30 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 4-ethenyl-2,3-dihydro-1H-isoindole-2-carboxylate as a yellow solid (4.00 g, 60%). LCMS (ES, m/z) 190 [M+H-Bu]⁺.

### Step 2. Tert-butyl 4-formyl-2,3-dihydro-1H-isoindole-2-carboxylate

To a solution of tert-butyl 4-ethenyl-2,3-dihydro-1H-isoindole-2-carboxylate (4.00 g, 16.3 mmol) in tetrahydrofuran (40 mL), water (20 mL) was added sodium periodate (6.93 g, 32.5 mmol), and Osmium (VIII) oxide (413 mg, 1.63 mmol). The resulting solution was stirred for 2 h at room temperature. The reaction mixture was poured into water (20 mL) and extracted with ethyl acetate (3×50 mL). The combined organic layers were dried over anhydrous sodium sulfate, filtered, and concentrated under vacuum. The resulting crude product was purified by silica gel chromatography (eluting with 1:3 ethyl acetate/petroleum ether) to afford tert-butyl 4-formyl-2,3-dihydro-1H-isoindole-2-carboxylate as a yellow solid (2.50 g, 60%). LCMS (ES, m/z) 192 [M+H-Bu]⁺.

### Intermediate 22. 3-[[(tert-butoxy)carbonyl](methyl)amino]-2-(3-chloro-4,5-difluorophenyl) propanoic Acid

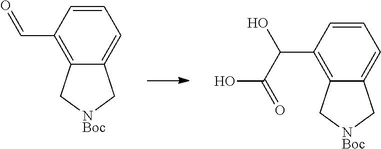 [SMILES: CC(C)(C)OC(=O)N1Cc2cccc(C=O)c2C1 ; CC(C)(C)OC(=O)N1Cc2cccc(C(O)C(=O)O)c2C1]

To a solution of tert-butyl 4-formyl-2,3-dihydro-1H-isoindole-2-carboxylate (2.50 g, 10.1 mmol) in 1,4-dioxane (8.1 mL) was added water (8.1 mL), potassium hydroxide (2.25 g, 40.3 mmol), tribromomethane (2.54 g, 10.1 mmol), and lithium chloride (847 mg, 20.2 mmol) at 0° C. The resulting mixture was stirred for 24 h at room temperature and then another 24 h at 35° C. while keeping pH value >12. The reaction mixture was washed with diethyl ether (50 mL) and then acidified to pH=1 with saturated citric acid. The product was extracted with ethyl acetate (2×50 mL). The combined organic layers were dried over anhydrous sodium sulfate, filtered, and concentrated under vacuum. The crude product was purified by prep-HPLC (Column: XBridge Shield RP18 OBD Column, 5 μm, 30×150 mm; Mobile Phase, A: water (containing 10 mmol/L NH₄HCO₃) and B: CH₃CN (5% to 40% B over 7 min); Flow rate: 60 mL/min; Detector: UV 254 nm) to afford 3-[[(tert-butoxy)carbonyl](methyl)amino]-2-(3-chloro-4,5-difluorophenyl)propanoic acid as a white solid (580 mg, 5%). LCMS (ES, m/z) 294 [M+H]⁺.

The Intermediates in Table 8 were synthesized according to the procedure described for Intermediate 22 above.

TABLE 8

| | | LCMS: (ESI) |
|---|---|---|
| Intermediate | Structure and Name | m/z [M + H]⁺ |
| 22-2B | 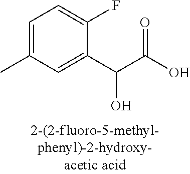 | 185 |

### Intermediate 22-1. Ethyl 2-[2-methylimidazo[1,2-a]pyridin-8-yl]prop-2-enoate

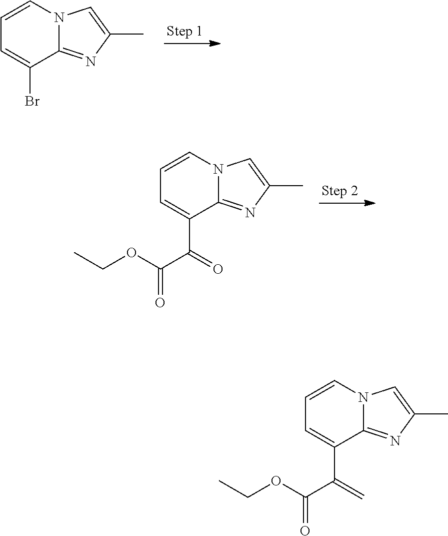 [SMILES: Cc1cn2cccc(Br)c2n1 ; CCOC(=O)C(=O)c1cccn2cc(C)nc12 ; C=C(C(=O)OCC)c1cccn2cc(C)nc12]

### Step 1. Ethyl 2-[2-methylimidazo[1,2-a]pyridin-8-yl]-2-oxoacetate

To a solution of 8-bromo-2-methylimidazo[1,2-a]pyridine (1.00 g, 4.64 mmol) in tetrahydrofuran (15 mL) was added n-BuLi (2.30 mL, 2.5 M in THF) dropwise with stirring at −78° C. The resulting solution was stirred for 10 min before adding diethyl oxalate (1.30 mL, 9.58 mmol). The mixture was stirred for 1 h at −78° C. and then quenched by addition of saturated aqueous ammonium chloride solution (5 mL). The product was extracted with ethyl acetate (3×15 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford ethyl 2-[2-methylimidazo[1,2-a]pyridin-8-yl]-2-oxoacetate as a yellow oil (565 mg, 50%). LCMS (ES, m/z): 233 [M+H]⁺.

### Step 2. Ethyl 2-[2-methylimidazo[1,2-a]pyridin-8-yl]prop-2-enoate

To a solution of bromo(methyl)triphenyl-lambda5-phosphane (292 mg, 0.82 mmol) in THF (2 mL) was added LiHMDS (137 mg, 0.82 mmol) at −78° C. The mixture was stirred for 20 min at room temperature and added into a solution of ethyl 2-[2-methylimidazo[1,2-a]pyridin-8-yl]-2-oxoacetate (200 mg, 0.82 mmol) in THF (2 mL) at 70° C. The resulting mixture was stirred for 2 h at 70° C. and warmed to room temperature. The reaction mixture was poured into saturated aqueous ammonium chloride solution (10 mL) and then extracted with ethyl acetate (3×10 mL). The combined organic layers were dried over anhydrous sodium sulfate, filtered, and concentrated under vacuum. The resulting crude product was purified by prep-TLC (eluting with 1:1 ethyl acetate/petroleum ether) to afford ethyl 2-[2-methylimidazo[1,2-a]pyridin-8-yl]prop-2-enoate as a yellow oil (35 mg, 19%). LCMS (ES, m/z): 231[M+H]⁺.

The Intermediates in Table 9 were synthesized according to the procedure described for Intermediate 22-1 above.

TABLE 9

| | | LCMS: |
|---|---|---|
| | | (ESI) m/z |
| Intermediate | Compound | [M + H] |
| 22-2 | 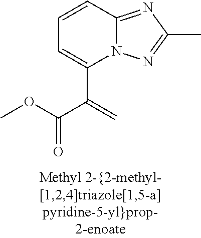 | 218 |
| 22-3 | 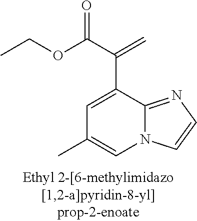 [SMILES: C=C(C(=O)OCC)c1cc(C)cn2ccnc12] | 231 |
| 22-4 | 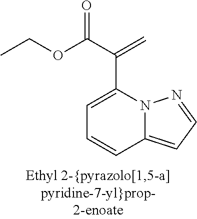 [SMILES: C=C(C(=O)OCC)c1cccc2ccnn12] | 217 |

### Intermediate 23-1 4-bromo-6-methoxy-2-methyl-1,3-benzothiazole

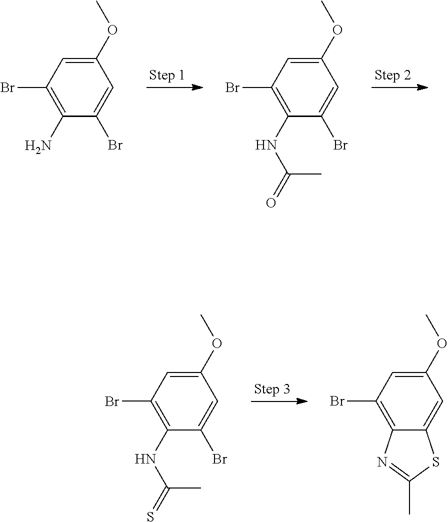

### Step 1. N-(2,6-dibromo-4-methoxyphenyl)acetamide

To a solution of 2,6-dibromo-4-methoxyaniline (5.00 g, 17.4 mmol) in acetic acid (30 mL) was added acetic anhydride (2.18 mL, 22.7 mmol). The solution was stirred for 30 min at 90° C. and cooled to room temperature. The reaction mixture was poured into ice water (30 mL). The resulting solids were collected by filtration, washed with water (2×30 mL), and dried under vacuum to afford N-(2,6-dibromo-4-methoxyphenyl)acetamide as a light yellow solid (3.20 g, 48%). LCMS (ES, m/z): 322, 324, 326 [M+H]⁺.

### Step 2. N-(2,6-dibromo-4-methoxyphenyl)ethanethioamide

To a solution of N-(2,6-dibromo-4-methoxyphenyl)acetamide (3.20 g, 8.42 mmol) in toluene (100 mL) was added Lawesson's Reagent (1.74 g, 4.21 mmol). The resulting mixture was stirred for 3 h at 110° C. and then cooled to room temperature. The mixture was concentrated under vacuum. The resulting crude product was purified by silica gel chromatography (eluting with 1:2 ethyl acetate/petroleum ether) to afford N-(2,6-dibromo-4-methoxyphenyl)ethanethioamide as a white solid (2.30 g, 68%). LCMS (ES, m/z): 338, 340, 342 [M+H]⁺.

### Step 3. 4-bromo-6-methoxy-2-methyl-1,3-benzothiazole

To a solution of N-(2,6-dibromo-4-methoxyphenyl)ethanethioamide (2.50 g, 6.27 mmol) in 1,4-dioxane (16 mL) was added cesium carbonate (3.60 g, 10.8 mmol), copper (I) iodide (70 mg, 0.36 mmol), 1,10-phenanthroline (132 mg, 0.72 mmol). The resulting mixture was stirred for 16 h at 85° C. and cooled to room temperature. The reaction mixture was filtered, poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 1:3 ethyl acetate/petroleum ether) to afford 4-bromo-6-methoxy-2-methyl-1,3-benzothiazole as a white solid (1.10 g, 58%). LCMS (ES, m/z): 258, 260 [M+H]⁺.

### Intermediate 24-1. 4-Bromo-7-methyl-2,1,3-benzothiadiazole

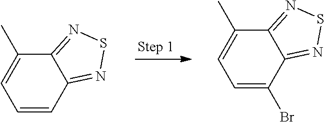

To 4-methyl-2,1,3-benzothiadiazole (4.00 g, 25.8 mmol) was added hydrogen bromide (40 mL, 40% in acetic acid) followed by addition of bromine (1.38 mL, 25.8 mmol) at 0° C. The mixture was stirred for 16 h at 120° C. and then cooled to room temperature. The reaction mixture was poured into water (200 mL) and then extracted with ethyl acetate (3×50 mL). The combined organic layers were dried over anhydrous sodium sulfate, filtered, and concentrated under vacuum to afford 4-bromo-7-methyl-2,1,3-benzothiadiazole as a light yellow solid (2.80 g, 45%). LCMS (ES, m/z): 229, 231 [M+H]⁺.

### Intermediate 25-1. 5-Bromo-2-methyl-[1,2,4]triazolo[1,5-a]pyridine

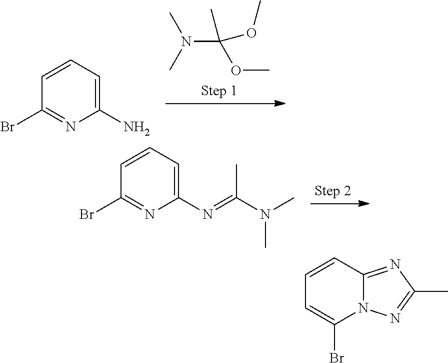

### Step 1. (E)-N′-(6-bromopyridin-2-yl)-N,N-dimethylethanimidamide

To a solution of 6-bromopyridin-2-amine (5.00 g, 28.9 mmol) in DMF (50 mL) was added (1,1-dimethoxyethyl)dimethylamine (12.0 g, 90.1 mmol). The resulting mixture was stirred for 16 h at 140° C. and then cooled to room temperature. The reaction mixture was concentrated under vacuum to afford (E)-N′-(6-bromopyridin-2-yl)-N,N-dimethylethanimidamide as a light yellow solid (6.90 g, crude). LCMS (ES, m/z): 242, 244 [M+H]⁺.

### Step 2. 5-Bromo-2-methyl-[1,2,4]triazolo[1,5-a]pyridine

To a solution of (E)-N′-(6-bromopyridin-2-yl)-N,N-dimethylethanimidamide (6.90 g, 28.5 mmol) in methanol (50 mL) was added pyridine (4.58 mL, 56.9 mmol) and (aminooxy)sulfonic acid (4.50 g, 39.9 mmol). The resulting mixture was stirred for 16 h at room temperature. The reaction mixture was poured into water (20 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford 5-bromo-2-methyl-[1,2,4]triazolo[1,5-a]pyridine as a light yellow solid (1.50 g, 25%). LCMS (ES, m/z): 212, 214 [M+H]⁺.

### Intermediate 26-1. 3-[[(tert-butoxy)carbonyl]amino]-2-cyclohexylpropanoic Acid

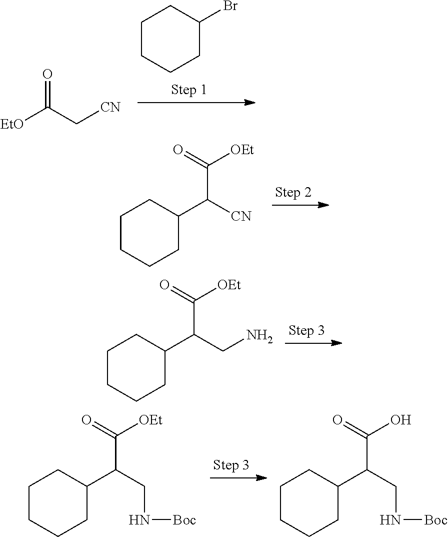

### Step 1. Ethyl 2-cyano-2-cyclohexylacetate

To a solution of ethyl 2-cyanoacetate (1.00 g, 8.84 mmol) in DMF (10 mL) was added potassium t-butoxide (991 mg, 8.83 mmol). The resulting mixture was stirred for 30 min at room temperature and was added bromocyclohexane (1.20 g, 7.36 mmol). The resulting mixture was stirred for 16 h at 100° C. and cooled to room temperature. The reaction mixture was poured into water (10 mL) and then extracted with ethyl acetate (3×10 mL). The combined organic layers were dried over anhydrous sodium sulfate, filtered and concentrated under vacuum. The resulting crude product was purified by silica gel chromatography (eluting with 1:10 ethyl acetate/petroleum ether) to afford ethyl 2-cyano-2-cyclohexylacetate as a light yellow oil (470 mg, 27%). LCMS (ES, m/z): 196 [M+H]⁺.

### Step 2. Ethyl 3-amino-2-cyclohexylpropanoate

To a solution of ethyl 2-cyano-2-cyclohexylacetate (470 mg, 2.41 mmol) in ethanol (5 mL) was added Pd/C (50 mg, 10 wt % Pd on active carbon). The resulting mixture was stirred for 16 h at room temperature under hydrogen (2-3 atm). The reaction mixture was filtered and concentrated under vacuum. The resulting crude product was purified by silica gel chromatography (eluting with 1:5 ethyl acetate/petroleum ether) to afford ethyl 3-amino-2-cyclohexylpropanoate as a light yellow oil (420 mg, 88%). LCMS (ES, m/z): 200 [M+H]⁺.

### Step 3. Ethyl 3-[[(tert-butoxy)carbonyl]amino]-2-cyclohexylpropanoate

To a solution of ethyl 3-amino-2-cyclohexylpropanoate (420 mg, 2.11 mmol) in dichloromethane (6 mL) was added TEA (0.88 mL, 6.32 mmol), and di-tert-butyl dicarbonate (920 mg, 4.22 mmol). The resulting mixture was stirred for 2 h at room temperature. The reaction mixture was poured into water (10 mL) and then extracted with dichloromethane (3×10 mL). The combined organic layers were dried over anhydrous sodium sulfate, filtered and concentrated under vacuum. The resulting crude product was purified by silica gel chromatography (eluting with 1:4 ethyl acetate/petroleum ether) to afford ethyl 3-[[(tert-butoxy)carbonyl]amino]-2-cyclohexylpropanoate as a light yellow solid (510 mg, 80%). LCMS (ES, m/z): 300 [M+H]⁺.

### Step 4. 3-[[(tert-butoxy)carbonyl]amino]-2-cyclohexylpropanoic Acid

To a solution of ethyl 3-[[(tert-butoxy)carbonyl]amino]-2-cyclohexylpropanoate (250 mg, 0.83 mmol) in tetrahydrofuran (1.5 mL) and water (1.5 mL) was added lithium hydroxide (100 mg, 4.18 mmol). The resulting mixture was stirred for 16 h at 50° C. and cooled to room temperature. The reaction mixture was washed with diethyl ether (5 mL) and then acidified to pH=5 with citric acid solution (10%). The resulting solution was extracted with ethyl acetate (3×5 mL). The combined organic layers were dried over anhydrous sodium sulfate, filtered and concentrated under vacuum to afford 3-[[(tert-butoxy)carbonyl]amino]-2-cyclohexylpropanoic acid as light yellow oil (110 mg, 49%). LCMS (ES, m/z): 272 [M+H]⁺.

### Intermediate 27-1. Methyl 2-(3-cyclopropyl-4,5-difluorophenyl)prop-2-enoate

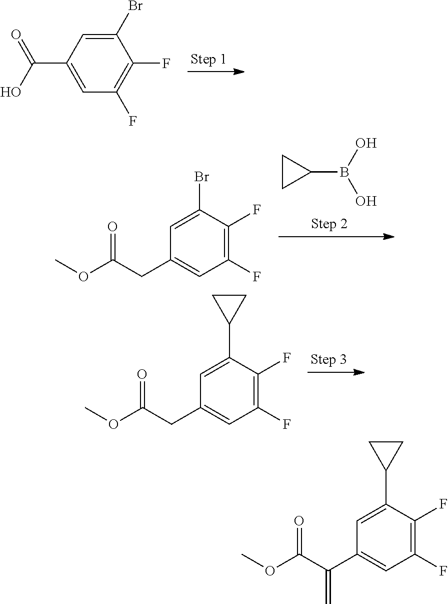

### Step 1. Methyl 2-(3-bromo-4,5-difluorophenyl)acetate

To a solution of 3-bromo-4,5-difluorobenzoic acid (2.50 g, 10.6 mmol) in toluene (15 mL), was added thionyl chloride (15 mL). The resulting solution was refluxed for 3 h, then cooled to room temperature and concentrated under vacuum. The resulting mixture was dissolved in THF (15 mL) and treated with triethylamine (2.47 mL, 17.9 mmol) and (diazomethyl)trimethylsilane (8.8 mL, 2.0 M in THF) at 0° C. The resulting mixture was stirred for 16 h at room temperature and then poured into saturated aqueous sodium bicarbonate (50 mL) and extracted with ethyl acetate (3×100 mL). The combined organic layers were dried over anhydrous sodium sulfate, filtered, and concentrated under vacuum. The resulting mixture was dissolved in methanol (40 mL) and treated with triethylamine (2.47 mL, 17.9 mmol) and silver (I) benzoate (1.40 g, 6.33 mmol) at 0° C. The mixture was stirred for 16 h at room temperature and then concentrated under vacuum. The crude product was purified by silica gel chromatography (eluting with 0:100 to 10:90 ethyl acetate/petroleum ether) to afford methyl 2-(3-bromo-4,5-difluorophenyl)acetate as a colorless oil (0.98 g, 35%). LCMS (ES, m/z): 265, 267 [M+H]⁺.

### Step 2. Methyl 2-(3-cyclopropyl-4,5-difluorophenyl)acetate

To a solution of methyl 2-(3-bromo-4,5-difluorophenyl)acetate (1.70 g, 6.72 mmol) in 1,4-dioxane (40 mL) was added cyclopropylboronic acid (865 mg, 10.1 mmol), potassium phosphate (4.20 g, 20.1 mmol), Pd(dppf)Cl₂ (246 mg, 0.34 mmol) and water (8 mL). The mixture was stirred for 16 h at 90° C. and cooled to room temperature. The reaction mixture was poured into water (50 mL) and then extracted with ethyl acetate (3×50 mL). The combined organic layers were dried over anhydrous sodium sulfate, filtered, and concentrated under vacuum. The crude product was purified by silica gel chromatography (eluting with 0:100 to 30:70 ethyl acetate/petroleum ether) to afford methyl 2-(3-cyclopropyl-4,5-difluorophenyl)acetate as a colorless oil (550 mg, 36%). LCMS (ES, m/z): 227 [M+H]⁺.

### Step 3. Methyl 2-(3-cyclopropyl-4,5-difluorophenyl)prop-2-enoate

To a solution of methyl 2-(3-cyclopropyl-4,5-difluorophenyl)acetate (550 mg, 2.43 mmol) in DMF (15 mL), was added potassium carbonate (840 mg, 6.08 mmol), tetrabutylammonium iodide (90 mg, 0.24 mmol) and paraformaldehyde (263 mg, 2.92 mmol). The resulting mixture was stirred for 10 min at 60° C. and then cooled to room temperature. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford methyl 2-(3-cyclopropyl-4,5-difluorophenyl)prop-2-enoate as a colorless oil (159 mg, 27%). LCMS (ES, m/z): 239 [M+H]⁺.

### Intermediate 28-1. Ethyl 2-(2-bromo-1,3-thiazol-4-yl)prop-2-enoate

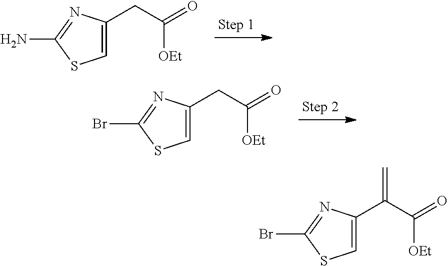

### Step 1. Ethyl 2-(2-bromo-1,3-thiazol-4-yl)acetate

To a solution of ethyl 2-(2-amino-1,3-thiazol-4-yl)acetate (5.00 g, 26.3 mmol) in MeCN (50 mL) was added tert-butyl nitrite (4.80 mL, 40.0 mmol) and copper (I) bromide (3.77 g, 26.3 mmol) at 60° C. The mixture was stirred for 2 h at 75° C. and then cooled to room temperature. The reaction mixture was concentrated under vacuum. The crude product was purified by silica gel chromatography (eluting with 0:100 to 10:90 ethyl acetate/petroleum ether) to afford ethyl 2-(2-bromo-1,3-thiazol-4-yl)acetate as a yellow oil (2.70 g, 41%). LCMS (ES, m/z): 250, 252 [M+H]⁺.

### Step 2. Ethyl 2-(2-bromo-1,3-thiazol-4-yl)prop-2-enoate

To a solution of ethyl 2-(2-bromo-1,3-thiazol-4-yl)acetate (2.50 g, 9.50 mmol) in DMF (20 mL) was added paraformaldehyde (1.20 g, 13.3 mmol), potassium carbonate (3.45 g, 25.0 mmol), and tetrabutylammonium iodide (369 mg, 1.00 mmol). The resulting mixture was stirred for 10 min at 60° C. and then cooled to room temperature. The reaction mixture was poured into water (50 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 1:100 ethyl acetate/petroleum ether) to afford ethyl 2-(2-bromo-1,3-thiazol-4-yl)prop-2-enoate as a yellow oil (350 mg, 14%). LCMS (ES, m/z): 262, 264 [M+H]⁺.

### Intermediate 29-1. Ethyl 3-[[(tert-butoxy)carbonyl](methyl)amino]-2-(2-cyclopropyl-1,3-thiazol-4-yl)propanoate

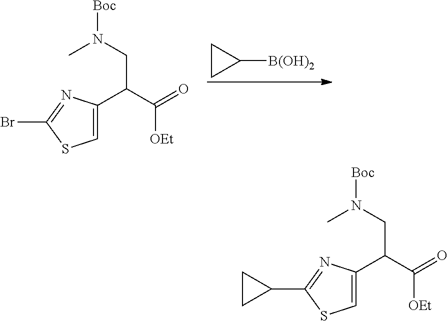

To a solution of ethyl 2-(2-bromo-1,3-thiazol-4-yl)-3-[[(tert-butoxy)carbonyl](methyl)amino]propanoate (350 mg, 0.85 mmol) in 1,4-dioxane (10 mL) and water (1 mL) was added cyclopropylboronic acid (115 mg, 1.31 mmol), potassium phosphate (566 mg, 2.67 mmol), and Pd(dppf)Cl₂ (33 mg, 0.05 mmol). The resulting mixture was stirred for 18 h at 100° C. and then cooled to room temperature. The reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford ethyl 3-[[(tert-butoxy)carbonyl](methyl)amino]-2-(2-cyclopropyl-1,3-thiazol-4-yl)propanoate as a yellow oil (180 mg, 60%). LCMS (ES, m/z): 355 [M+H]⁺.

### Intermediate 30-1. 3-[[(tert-butoxy)carbonyl]amino]-2-(2-methyl-1,3-benzothiazol-4-yl)propanoic Acid

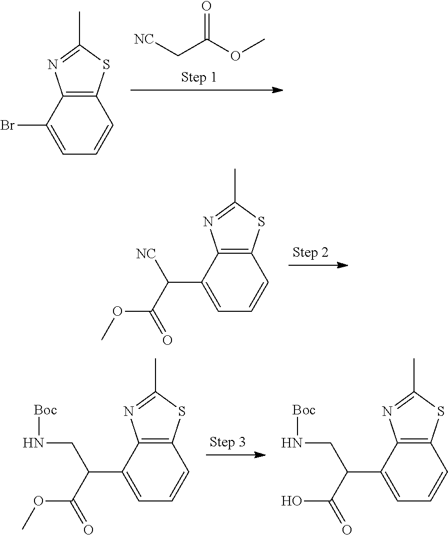

### Step 1. Methyl 2-cyano-2-(2-methyl-1,3-benzothiazol-4-yl)acetate

To a solution of 4-bromo-2-methyl-1,3-benzothiazole (1.00 g, 4.40 mmol) in toluene (10 mL) was added methyl 2-cyanoacetate (1.30 g, 13.1 mmol), Pd₂(dba)₃.CHCl₃(227 mg, 0.22 mmol), P(t-Bu)₃.HBF₄(318 mg, 1.09 mmol) and sodium phosphate (2.19 g, 0.01 mol). The resulting mixture was irradiated in a microwave for 3 h at 125° C. and cooled to room temperature. The reaction was repeated three times and the batches were combined and poured into water (10 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford methyl 2-cyano-2-(2-methyl-1,3-benzothiazol-4-yl)acetate as a yellow oil (1.80 g, 44%). LCMS (ES, m/z): 247 [M+H]⁺.

### Step 2. Methyl 3-(tert-butoxycarbonylamino)-2-(2-methylbenzo[d]thiazol-4-yl)propanoate

To a solution of methyl 2-cyano-2-(2-methyl-1,3-benzothiazol-4-yl)acetate (1.80 g, 7.31 mmol) in methanol (90 mL) and tetrahydrofuran (90 mL) was added TEA (5.08 mL, 36.6 mmol), di-tert-butyl dicarbonate (3.19 g, 14.6 mmol) and Raney Ni (800 mg). The resulting mixture was stirred for 16 h at room temperature under hydrogen atmosphere (2-3 atm). The reaction mixture was filtered and concentrated under vacuum. The resulting crude product was purified by silica gel chromatography (eluting with 0:100 to 30:70 ethyl acetate/petroleum ether) to afford methyl 3-(tert-butoxycarbonylamino)-2-(2-methylbenzo[d]thiazol-4-yl)propanoate as yellow oil (730 mg, 27%). LCMS (ES, m/z): 351 [M+H]⁺.

### Step 3. 3-[[(tert-butoxy)carbonyl]amino]-2-(2-methyl-1,3-benzothiazol-4-yl)propanoic Acid

To a solution of methyl 3-[[(tert-butoxy)carbonyl]amino]-2-(2-methyl-1,3-benzothiazol-4-yl)propanoate (730 mg, 1.90 mmol) in tetrahydrofuran (4 mL) and water (4 mL) was added lithium hydroxide (251 mg, 10.5 mmol). The resulting mixture was stirred for 16 h at room temperature. The reaction mixture was washed with diethyl ether (1×8 mL) and then acidified to pH=5 with saturated citric acid. The resulting solution was extracted with ethyl acetate (2×8 mL). The organic layers were combined, dried over anhydrous sodium sulfate, filtered, and concentrated under vacuum to afford 3-[[(tert-butoxy)carbonyl]amino]-2-(2-methyl-1,3-benzothiazol-4-yl)propanoic acid as a yellow oil (570 mg, 86%). LCMS (ES, m/z): 337 [M+H]⁺.

### Intermediate 31-1. 4-bromo-2-methyl-1,3-benzoxazole

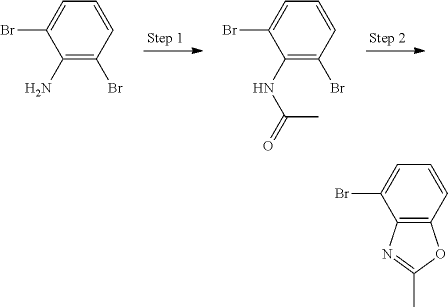

### Step 1. N-(2,6-dibromophenyl)acetamide

To a solution of 2,6-dibromoaniline (5.00 g, 19.5 mmol) in AcOH (50 mL) was added acetyl acetate (2.78 mL, 29.3 mmol). The resulting mixture was stirred for 1 h at 90° C. and then cooled to room temperature. The reaction was quenched in ice water (100 mL). The solids were collected by filtration and dried under vacuum to afford N-(2,6-dibromophenyl)acetamide as a white solid (2.00 g, 33%). LCMS (ES, m/z): 292, 294, 296 [M+H]⁺.

### Step 2. 4-bromo-2-methyl-1,3-benzoxazole

To a solution of N-(2,6-dibromophenyl)acetamide (2.00 g, 6.49 mmol) in 1,4-dioxane (20 mL) was added copper (I) iodide (124 mg, 0.65 mmol), 1,10-phenanthroline (117 mg, 0.65 mmol), and potassium carbonate (1.79 g, 12.9 mmol). The resulting mixture was stirred for 16 h at 90° C. and then cooled to room temperature. The reaction mixture was poured into water (50 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 4-bromo-2-methyl-1,3-benzoxazole as a yellow solid (900 mg, 65%). LCMS (ES, m/z): 212, 214 [M+H]⁺.

### Intermediate 32-1. 4-Bromo-7-fluoro-2-methyl-1,3-benzothiazole

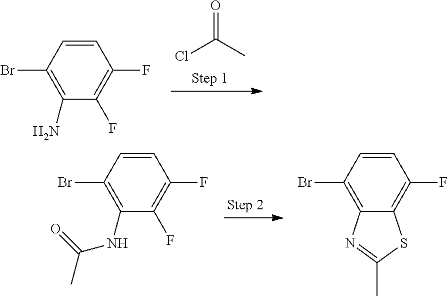

### Step 1. N-(6-bromo-2,3-difluorophenyl)acetamide

To a solution of 6-bromo-2,3-difluoroaniline (4.00 g, 16.4 mmol) in dichloromethane (40 mL) was added 4-dimethylaminopyridine (116 mg, 0.90 mmol), TEA (3.64 mL, 27.5 mmol), and acetyl chloride (1.98 g, 24 mmol). The resulting mixture was stirred for 2 h at room temperature. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 1:5 ethyl acetate/petroleum ether) to afford N-(6-bromo-2,3-difluorophenyl)acetamide as yellow oil (4.30 g, 92%). LCMS (ES, m/z): 250, 252 [M+H]⁺.

### Step 2. 4-Bromo-7-fluoro-2-methyl-1,3-benzothiazole

To a solution of N-(6-bromo-2,3-difluorophenyl)acetamide (2.50 g, 8.50 mmol) in toluene (20 mL) was added Lawesson reagent (2.43 g, 5.71 mmol). The resulting mixture was stirred for 4 h at 110° C. and then cooled to room temperature. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 1:4 ethyl acetate/petroleum ether) to afford 4-bromo-7-fluoro-2-methyl-1,3-benzothiazole as a yellow solid (2.20 g, 89%). LCMS (ES, m/z): 246, 248 [M+H]⁺.

### Intermediate 33-1. 2-{1-[(tert-butoxy)carbonyl]pyrrolidin-2-yl}-2-(3-chlorophenyl)acetic Acid

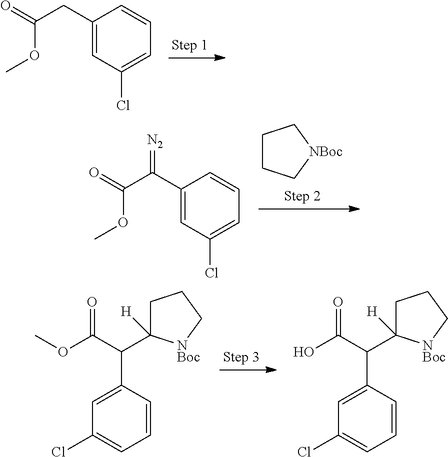

### Step 1. Methyl 2-(3-chlorophenyl)-2-diazoacetate

To a solution of methyl 2-(3-chlorophenyl)acetate (5.00 g, 25.7 mmol) in CH₃CN (50 mL) was added 1,8-diazabicyclo[5.4.0]undec-7-ene (4.87 mL, 32.6 mmol) dropwise with stirring at 0° C. followed by the addition of 4-methylbenzene-1-sulfonyl azide (6.40 g, 32.5 mmol) added dropwise with stirring at 0° C. The solution was stirred for 4 h at 25° C. The reaction mixture was treated with water (50 mL) and extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 ethyl acetate/dichloromethane) to afford methyl 2-(3-chlorophenyl)-2-diazoacetate as a yellow solid (5.00 g, 83%). LCMS (ES, m/z): 211, 213 [M+H]⁺.

### Step 2. Tert-butyl 2-[1-(3-chlorophenyl)-2-methoxy-2-oxoethyl]pyrrolidine-1-carboxylate

To a solution of tert-butyl pyrrolidine-1-carboxylate (894 mg, 5.22 mmol) in hexane (150 mL) was added tetrakis [(R)-(+)-N—(P-dodecylphenylsulfonyl)prolinato]dirhodium (II) (49 mg, 0.026 mmol) followed by treatment with methyl 2-(3-chlorophenyl)-2-diazoacetate (550 mg, 2.61 mmol) as a solution in hexane (100 mL) over 60 min with stirring at −50° C. The resulting solution was stirred for 10 h at −50° C. and then 16 h at room temperature. The reaction was poured into saturated ammonium chloride solution (100 mL) and then extracted with ethyl acetate (3×150 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 2-[1-(3-chlorophenyl)-2-methoxy-2-oxoethyl]pyrrolidine-1-carboxylate as a yellow solid (400 mg, 39%). LCMS (ES, m/z): 354, 356 [M+H]⁺.

### Step 3. 2-{1-[(tert-butoxy)carbonyl]pyrrolidin-2-yl}-2-(3-chlorophenyl)acetic Acid

To a solution of tert-butyl 2-[1-(3-chlorophenyl)-2-methoxy-2-oxoethyl]pyrrolidine-1-carboxylate (400 mg, 1.13 mmol) in tetrahydrofuran (20 mL) and water (5 mL) was added lithium hydroxide (135 mg, 5.65 mmol). The resulting mixture was stirred for 18 h at room temperature. The reaction mixture was washed with diethyl ether (1×10 mL) and then acidified to pH=6 with saturated citric acid. The resulting solution was extracted with ethyl acetate (3×20 mL). The combined organic layers were dried over anhydrous sodium sulfate, filtered, and concentrated under vacuum to afford 2-{1-[(tert-butoxy)carbonyl]pyrrolidin-2-yl}-2-(3-chlorophenyl)acetic acid as yellow oil (300 mg, 78%). LCMS (ES, m/z): 340, 342 [M+H]⁺.

The Intermediates in Table 10 were synthesized according to the procedure described for Intermediate 33-1 above.

TABLE 10

| | | LCMS: (ESI) |
|---|---|---|
| Intermediate | Structure and Name | m/z [M + H]⁺ |
| 33-2 | 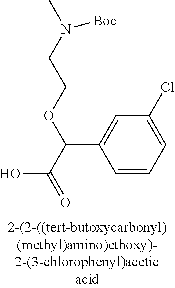 [SMILES: CN(CCOC(C(=O)O)c1cccc(Cl)c1)C(=O)OC(C)(C)C] | 344 |

### Intermediate 34-1. 3-[(oxetan-3-yl)amino]-2-phenylpropanoic Acid

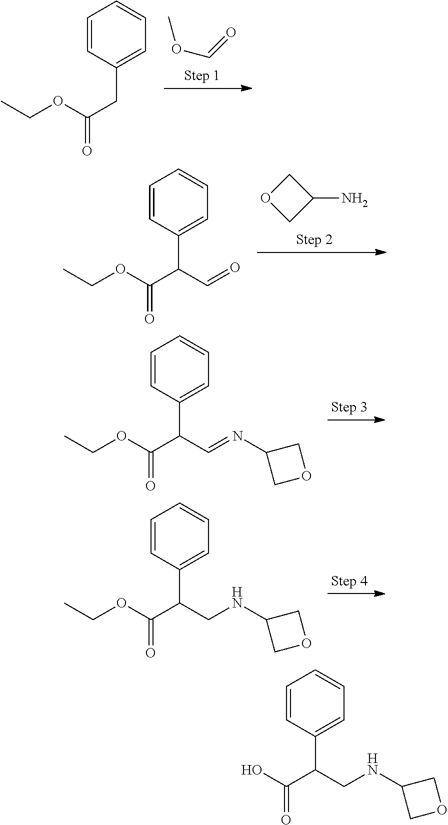

### Step 1. Ethyl 3-oxo-2-phenylpropanoate

To a solution of ethyl 2-phenylacetate (5.00 g, 30.4 mmol) in toluene (25 mL) was added sodium hydride (2.20 g, 91.7 mmol, 60% dispersion in mineral oil) in portions with stirring at 0° C. The resulting mixture was stirred for 1 h at 0° C. and treated with methyl formate (9.34 mL, 152 mmol). The mixture was stirred for 16 h at 25° C. The reaction was treated with saturation aqueous ammonium chloride (20 mL) and extracted with ethyl acetate (3×50 mL). The combined organic layers were dried over anhydrous sodium sulfate, filtered, and concentrated under vacuum. The crude product was purified by silica gel chromatography (eluting with 0:100 to 10:90 ethyl acetate/dichloromethane) to afford ethyl 3-oxo-2-phenylpropanoate as a yellow solid (2.00 g, 22%). LCMS (ES, m/z): 193 [M+H]⁺.

### Step 2. Ethyl (3E)-3-[(oxetan-3-yl)imino]-2-phenylpropanoate

To a solution of ethyl 3-oxo-2-phenylpropanoate (500 mg, 2.60 mmol) in dichloromethane (5 mL), was added oxetan-3-amine (949 mg, 12.9 mmol). The resulting mixture was stirred for 16 h at 25° C. The reaction was poured into water (10 mL) and then extracted with ethyl acetate (3×15 mL). The combined organic layers were dried over anhydrous sodium sulfate, filtered, and concentrated under vacuum. The crude product was purified by silica gel chromatography (eluting with 0:100 to 25:75 ethyl acetate/petroleum ether) to afford ethyl (3E)-3-[(oxetan-3-yl)imino]-2-phenylpropanoate as a yellow oil (350 mg, 44%). LCMS (ES, m/z): 248 [M+H]⁺.

### Step 3. Ethyl 3-[(oxetan-3-yl)amino]-2-phenylpropanoate

To a solution of ethyl 3-[(oxetan-3-yl)amino]-2-phenylpropanoate (350 mg, 1.40 mmol) in methanol (5 mL) was added palladium on activated carbon (35 mg, 10 wt %). The resulting mixture was stirred for 16 h at 25° C. under hydrogen atmosphere (2-3 atm). The reaction mixture was filtered and concentrated under vacuum to afford ethyl 3-[(oxetan-3-yl)amino]-2-phenylpropanoate as a yellow oil (100 mg, 28%). LCMS (ES, m/z): 250 [M+H]⁺.

### Step 4. 3-[(oxetan-3-yl)amino]-2-phenylpropanoic Acid

To a solution of ethyl 3-[(oxetan-3-yl)amino]-2-phenylpropanoate (100 mg, 0.36 mmol) in tetrahydrofuran (4 mL) and water (4 mL) was added lithium hydroxide (48 mg, 2.00 mmol). The resulting mixture was stirred for 6 h at room temperature. The reaction mixture was washed with diethyl ether (1×8 mL) and then acidified to pH=5 with saturated aqueous citric acid. The product was extracted with ethyl acetate (2×8 mL). The combined organic layers were dried over anhydrous sodium sulfate, filtered, and concentrated under vacuum to afford 3-[(oxetan-3-yl)amino]-2-phenylpropanoic acid as a yellow oil (60 mg, 68%). LCMS (ES, m/z): 222 [M+H]⁺.

### Intermediate 35-1. 3-(3-hydroxyazetidin-1-yl)-2-phenylpropanoic Acid

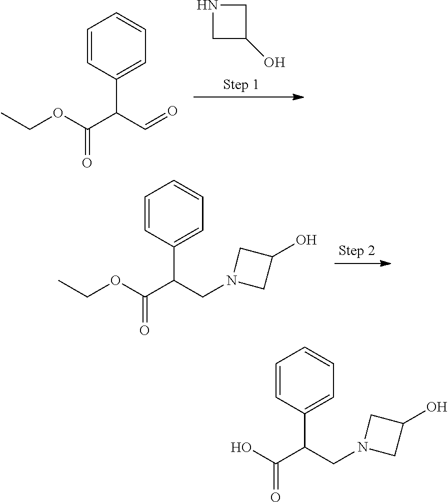 [SMILES: CCOC(=O)C(C=O)c1ccccc1 ; OC1CNC1 ; CCOC(=O)C(CN1CC(O)C1)c1ccccc1 ; O=C(O)C(CN1CC(O)C1)c1ccccc1]

### Step 1. Ethyl 3-(3-hydroxyazetidin-1-yl)-2-phenylpropanoate

To a solution of ethyl 3-oxo-2-phenylpropanoate (500 mg, 2.60 mmol) in dichloromethane (10 mL) was added azetidin-3-ol hydrochloride (1.45 g, 13.2 mmol). The resulting mixture was stirred for 2 hours and treated with sodium triacetoxyborohydride (1.65 g, 7.80 mmol). The mixture was stirred for 16 h at room temperature and then poured into water (5 mL). The resulting mixture was extracted with dichloromethane (3×5 mL). The combined organic layers were dried over anhydrous sodium sulfate, filtered, and concentrated under vacuum. The crude product was purified by prep-TLC (eluting with 1:5 ethyl acetate/petroleum ether) to afford ethyl 3-(3-hydroxyazetidin-1-yl)-2-phenylpropanoate as a yellow oil (300 mg, 46%). LCMS (ES, m/z): 250 [M+H]⁺.

### Step 2. 3-(3-hydroxyazetidin-1-yl)-2-phenylpropanoic Acid

To a solution of ethyl 3-(3-hydroxyazetidin-1-yl)-2-phenylpropanoate (200 mg, 0.72 mmol) in tetrahydrofuran (3 mL) was added water (3 mL), and lithium hydroxide (120 mg, 5.01 mmol). The resulting mixture was stirred for 16 h at room temperature. The reaction mixture was washed with diethyl ether (1×5 mL) and then acidified to pH=5 with saturated aqueous citric acid. The resulting solution was extracted with ethyl acetate (3×5 mL). The combined organic layers were dried over anhydrous sodium sulfate, filtered, and concentrated under vacuum to afford 3-(3-hydroxyazetidin-1-yl)-2-phenylpropanoic acid as a white solid (100 mg, 56%). LCMS (ES, m/z): 222 [M+H]⁺.

The Intermediates in Table 11 were synthesized according to the procedure described for Intermediate 35-1 above.

TABLE 11

| | | LCMS: (ESI) |
|---|---|---|
| Intermediate | Structure and Name | m/z [M + H]⁺ |
| 35-2 | 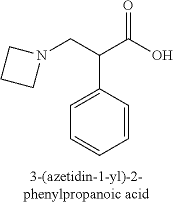 [SMILES: O=C(O)C(CN1CCC1)c1ccccc1] | 206 |

### Intermediate 36-1. 2-([1-[(tert-butoxy)carbonyl]azetidin-3-yl]oxy)-2-phenylacetic Acid

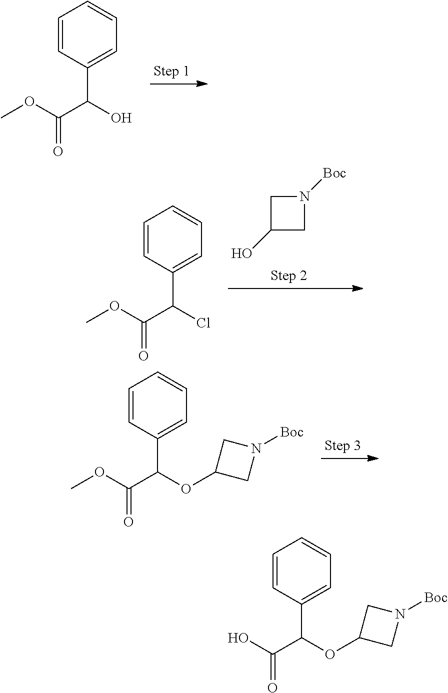

### Step 1. Methyl 2-chloro-2-phenylacetate

To methyl 2-hydroxy-2-phenylacetate (6.00 g, 35.8 mmol) was added sulfuryl chloride (30 mL). The reaction was stirred for 30 min at 80° C. and cooled to room temperature. The reaction was then poured into water (60 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford methyl 2-chloro-2-phenylacetate as a colorless oil (6.00 g, 91%). LCMS (ES, m/z): 185, 187 [M+H]⁺.

### Step 2. Tert-butyl 3-(2-methoxy-2-oxo-1-phenylethoxy)azetidine-1-carboxylate

To a solution of tert-butyl 3-hydroxyazetidine-1-carboxylate (6.20 g, 35.4 mmol) in tetrahydrofuran (200 mL) was added sodium hydride (1.90 g, 47.5 mmol, 60% dispersion in mineral oil) in portions with stirring at 0° C. After stirring for 30 min, 2-chloro-2-phenylacetate (6.00 g, 32.5 mmol) was added. The reaction was stirred for 5 h at 0° C. and then poured into water (100 mL). The product was extracted with ethyl acetate (3×200 mL). The combined organic layers were dried over anhydrous sodium sulfate, filtered, and concentrated under vacuum. The crude product was purified by silica gel chromatography (eluting with 0:100 to 100:0 ethyl acetate/petroleum ether) to afford tert-butyl 3-(2-methoxy-2-oxo-1-phenylethoxy)azetidine-1-carboxylate as a yellow oil (2.00 g, 19%). LCMS (ES, m/z): 322 [M+H]⁺.

### Step 3. 2-([1-[(tert-butoxy)carbonyl]azetidin-3-yl]oxy)-2-phenylacetic Acid

To a solution of tert-butyl 3-(2-methoxy-2-oxo-1-phenylethoxy)azetidine-1-carboxylate (2.00 g, 5.73 mmol) in tetrahydrofuran (15 mL) was added water (15 mL) and lithium hydroxide (747 mg, 31.2 mmol). The resulting mixture was stirred for 18 h at 25° C. The reaction mixture was diluted with water (10 mL) and then acidified to pH=4 with hydrochloric acid (1 N). The product was extracted with ethyl acetate (3×20 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 19×150 mm; Mobile Phase, A: water (containing 0.03% NH₃) and B: MeCN (5% to 50% over 30 min); Flow rate: 100 mL/min; Detector: UV 254 nm). The product fractions were concentrated and lyophilized to afford 2-([1-[(tert-butoxy)carbonyl]azetidin-3-yl]oxy)-2-phenylacetic acid as a yellow oil (700 mg, 40%). LCMS (ES, m/z): 308 [M+H]⁺.

### Intermediate 37-1. 2-[(1-[2-[(tert-butyldimethylsilyl)oxy]ethyl]azetidin-3-yl)oxy]-1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one

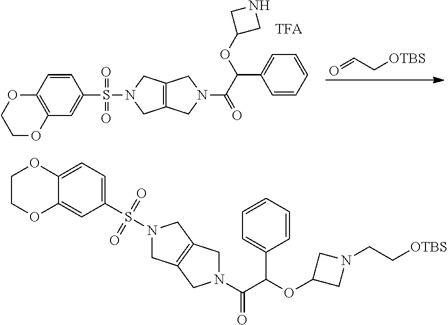

To a solution of 2-(azetidin-3-yloxy)-1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one TFA salt (330 mg, 0.52 mmol) in methanol (8 mL) was added acetic acid (2 mL) and 2-[(tert-butyldimethylsilyl)oxy]acetaldehyde (127 mg, 0.73 mmol). The resulting mixture was stirred for 1 hour at 25° C. Then it was added sodium triacetoxyborohydride (422 mg, 1.99 mmol). The resulting mixture was stirred for 18 h at 25° C. The reaction mixture was poured into water (10 mL) and then extracted with ethyl acetate (3×10 mL). The combined organic layers were dried over anhydrous sodium sulfate, filtered and concentrated under vacuum. The resulting crude product was purified by silica gel chromatography (eluting with 1:10 MeOH/DCM) to afford 2-[(1-[2-[(tert-butyldimethylsilyl)oxy]ethyl]azetidin-3-yl)oxy]-1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one as a yellow oil (150 mg, 44%). LCMS (ES, m/z): 656 [M+H]⁺.

### Intermediate 38-1. 1,2,3,4,5,6-hexahydropyrrolo[3,4-c]pyrrole Hydrogen Bromide Salt

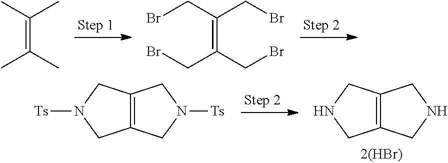

### Step 1. 1,4-dibromo-2,3-bis(bromomethyl)but-2-ene

To a solution of 2,3-dimethylbut-2-ene (1000 g, 11.9 mol) in DCM (500 mL) in 4 L 4-necked round bottom flask was added aqueous hydrogen bromide solution (150 mL, 48%). The reaction was treated with bromine (9.90 kg, 62.0 mol) while stirring at 0° C. and then heated to 45° C. in an oil bath and stirred for an additional 2 days. After cooling to room temperature, the reaction mixture was carefully poured into saturated sodium hydrogen sulfite solution (10 L). The precipitate was collected by filtration and dried in oven to afford 1,4-dibromo-2,3-bis(bromomethyl)but-2-ene as a light yellow solid (3345 g, 49%). GCMS: (EI, m/z): 398, 400, 402 [M]⁺.

### Step 2. 2,5-ditosyl-1,2,3,4,5,6-hexahydropyrrolo[3,4-c]pyrrole

To a solution of 1,4-dibromo-2,3-bis(bromomethyl)but-2-ene (2000 g, 3.50 mol) in DMF (20 L) was added 4-methylbenzene-1-sulfonamide (2137 g, 12.5 mol), and potassium carbonate (5175 g, 37.4 mol). The resulting mixture was stirred for 2 days at room temperature. The reaction mixture was then slowly poured into water/ice (20 L). The precipitate was collected by filtration, washed with ethanol and dried in oven to afford 2,5-ditosyl-1,2,3,4,5,6-hexahydropyrrolo[3,4-c]pyrrole as alight yellow solid (1345 g, 78%). LCMS: (ES, m/z): 419 [M+H]⁺.

### Step 3. 1,2,3,4,5,6-hexahydropyrrolo[3,4-c]pyrrole Hydro Bromide Salt

To a solution of 2,5-ditosyl-1,2,3,4,5,6-hexahydropyrrolo[3,4-c]pyrrole (1345 g, 2.73 mol) in aqueous hydrogen bromide solution (4500 mL, 48%) in 10 L 4-necked round-bottom flask, was added phenol (1270 g, 13.5 mol). The resulting mixture was stirred for 2 days at 120° C. After cooling to room temperature, the aqueous layer was collected and concentrated under vacuum. The resulting solids were washed with DCM/MeOH (v:v=10:1, 3×300 mL) and dried in an oven to afford 1,2,3,4,5,6-hexahydropyrrolo[3,4-c]pyrrole hydrogen bromide salt as a yellow solid (480 g, 61%). LCMS: (ES, m/z): 111 [M+H]⁺.

### Intermediate 39-1. 7-(4,5-dihydropyrrolo[3,4-c]pyrrol-2(1H,3H,4H)-ylsulfonyl)-2,3-dihydro-[1,4]dioxino[2,3-b]pyridine

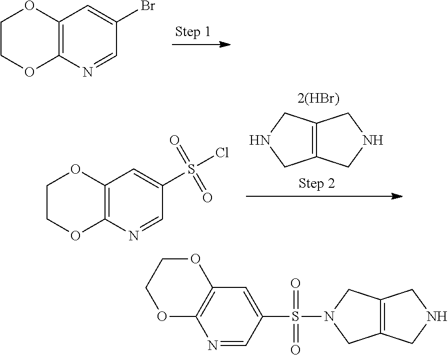

### Step 1. 2H,3H-[1,4]dioxino[2,3-b]pyridine-7-sulfonyl Chloride

To a solution of n-BuLi (2.0 mL, 2.5 M in hexane) was added n-Bu₂Mg (4.8 mL, 1.0 M in heptane). The resulting mixture was stirred for 10 min at room temperature. The reaction was treated with 7-bromo-2H,3H-[1,4]dioxino[2,3-b]pyridine (2.0 g, 9.26 mmol) in tetrahydrofuran (16 mL) added dropwise with stirring at −10° C. over a period of 10 min. The mixture was stirred for 1 h at −10° C. and then slowly added to a solution of sulfuryl dichloride (16 mL, 0.20 mol) in toluene (16 mL) at −10° C. and stirred for an additional 1 h. The reaction was quenched by the careful addition of saturated aqueous ammonium chloride solution (30 mL) at 0° C. The product was extracted with dichloromethane (3×50 mL). The organic layers were combined, dried over anhydrous sodium sulfate, filtered and concentrated under vacuum. The resulting crude product was purified by silica gel column (eluted with 1:3 ethyl acetate/petroleum ether) to afford 2H,3H-[1,4]dioxino[2,3-b]pyridine-7-sulfonyl chloride as a white solid (1.3 g, 60%). LCMS: (ES, m/z): 236, 238 [M+H]⁺.

### Step 2. 7-(4,5-dihydropyrrolo[3,4-c]pyrrol-2(1H,3H,4H)-ylsulfonyl)-2,3-dihydro-[1,4]dioxino[2,3-b]pyridine

To a solution of 1H-imidazole (14.5 g, 212 mmol) in dichloromethane (140 mL) was added 2H,3H-[1,4]dioxino[2,3-b]pyridine-7-sulfonyl chloride (25.0 g, 96 mmol) in dichloromethane (250 mL) dropwise with stirring at 0° C. The resulting mixture was stirred for 2 h at room temperature and then filtered and concentrated under vacuum. The solids were dissolved in absolute ethanol (125 mL) and added dropwise to a solution of 1H,2H,3H,4H,5H,6H-pyrrolo[3,4-c]pyrrole dihydrobromide (86.8 g, 319 mmol) in water (125 mL). The reaction was stirred for 18 h at room temperature and then 48 h at 60° C. After cooling to room temperature, the mixture was rendered basic (pH=14) with aqueous sodium hydroxide (50 wt %). The product was extracted with dichloromethane (3×300 mL). The organic layers were combined, dried over anhydrous sodium sulfate, filtered and concentrated under vacuum to afford of 2-[2H,3H-[1,4]dioxino[2,3-b]pyridine-7-sulfonyl]-1H,2H,3H,4H,5H,6H-pyrrolo[3,4-c]pyrrole as a yellow solid (13 g, 39.5%). LCMS: (ES, m/z): 310 [M+H]⁺.

### Intermediate 40-1. 2-(2-fluoro-5-methylphenyl)-2-hydroxyacetic Acid

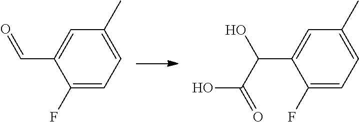

To a solution of potassium hydroxide (3.25 g, 58.0 mmol) in water (11.6 mL) was added LiCl (1.30 g, 29.0 mmol), 1,4-dioxane (11.6 mL), 2-fluoro-5-methylbenzaldehyde (2.00 g, 14.5 mmol) and tribromomethane (3.67 g, 14.5 mmol). The resulting mixture was stirred for 24 h at 5° C. and then for 24 h at 35° C. The reaction was washed with diethyl ether (1×8 mL) and then acidified to pH=1 with hydrochloric acid (1 N). The resulting solution was extracted with ethyl acetate (2×20 mL). The combined organic layers were dried over anhydrous sodium sulfate, filtered, and concentrated under vacuum to afford 2-(2-fluoro-5-methylphenyl)-2-hydroxyacetic acid as yellow oil (1.80 g, 60%). LCMS (ES, m/z): 183 [M−H]⁻.

### Intermediate 41-1. 2-(2-{[(tert-butoxy)carbonyl](methyl)amino}ethoxy)-2-(3-chloro-4,5-difluorophenyl)acetic Acid

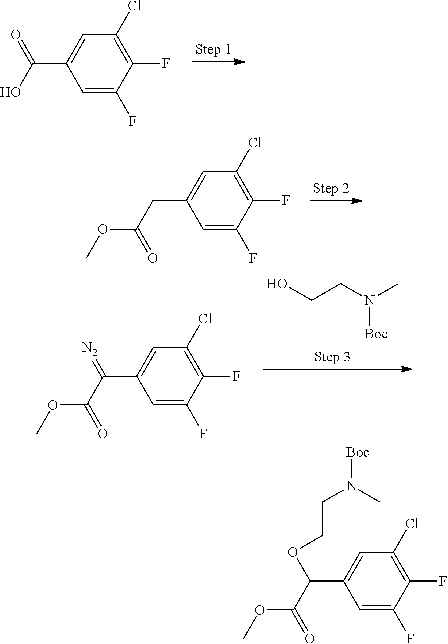

### Step 1. Methyl 2-(3-chloro-4,5-difluorophenyl)acetate

To a solution of 3-chloro-4,5-difluorobenzoic acid (3.00 g, 15.6 mmol) in toluene (30 mL), was added thionyl chloride (15 mL). The resulting solution was refluxed for 3 h, then cooled to room temperature and concentrated under vacuum. The resulting mixture was dissolved with THF (30 mL). The reaction was treated with triethylamine (3.70 mL, 26.5 mmol) and (diazomethyl)trimethylsilane (13.2 mL, 2.0 M in THF) at 0° C. and stirred for 16 h at room temperature. The reaction was poured into saturated aqueous sodium bicarbonate (50 mL) and extracted with ethyl acetate (3×100 mL). The combined organic layers were dried over anhydrous sodium sulfate, filtered, and concentrated under vacuum. The resulting mixture was dissolved in methanol (40 mL) and treated with triethylamine (3.70 mL, 26.5 mmol) and silver (I) benzoate (2.10 g, 9.35 mmol) at 0° C. The resulting mixture was stirred for 16 h at room temperature and then concentrated under vacuum. The crude product was purified by silica gel chromatography (eluting with 0:100 to 10:90 ethyl acetate/petroleum ether) to afford methyl 2-(3-chloro-4,5-difluorophenyl)acetate as a colorless oil (2.00 g, 58%). LCMS (ES, m/z): 221, 223 [M+H]⁺.

### Step 2. Methyl 2-(3-chloro-4,5-difluorophenyl)-2-diazoacetate

To a solution of methyl 2-(3-chloro-4,5-difluorophenyl)acetate (600 mg, 2.72 mmol) in MeCN (20 mL), was added DBU (496 mg, 3.26 mmol) and 4-methylbenzene-1-sulfonyl azide (751 mg, 3.81 mmol) at 0° C. The resulting solution was stirred for 16 h at room temperature. The reaction mixture was poured into water (25 mL) and then extracted with ethyl acetate (3×25 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford methyl 2-(3-chloro-4,5-difluorophenyl)-2-diazoacetate as a yellow solid (600 mg, 85%). LCMS (ES, m/z): 247, 249 [M+H]⁺.

### Step 3. Methyl 2-(2-{[(tert-butoxy)carbonyl](methyl)amino}ethoxy)-2-(3-chloro-4,5-difluorophenyl)acetate

To a solution of methyl 2-(3-chloro-4,5-difluorophenyl)-2-diazoacetate (689 mg, 2.79 mmol) in DCM (40 mL), was added tert-butyl N-(2-hydroxyethyl)-N-methylcarbamate (587 mg, 3.35 mmol), and Rhodium (II) acetate dimer (49 mg, 0.11 mmol). The resulting mixture was stirred for 16 h at room temperature and concentrated under vacuum. The resulting crude product was purified by silica gel chromatography (eluting with 0:100 to 10:90 ethyl acetate/petroleum ether) to afford methyl 2-(2-{[(tert-butoxy)carbonyl](methyl)amino}ethoxy)-2-(3-chloro-4,5-difluorophenyl)acetate as a light yellow oil (700 mg, 63%). LCMS (ES, m/z): 394, 396 [M+H]⁺.

### Step 4. 2-(2-{[(tert-butoxy)carbonyl](methyl)amino}ethoxy)-2-(3-chloro-4,5-difluorophenyl)acetic Acid

To a solution of methyl 2-(2-{[(tert-butoxy)carbonyl](methyl)amino}ethoxy)-2-(3-chloro-4,5-difluorophenyl)acetate (200 mg, 0.51 mmol) in THF (7 mL), was added water (7 mL) and lithium hydroxide (61 mg, 2.54 mmol). The resulting mixture was stirred for 16 h at 25° C. The reaction mixture was acidified to pH=6-7 with saturated citric acid and then extracted with ethyl acetate (3×15 mL). The combined organic layers were dried over anhydrous sodium sulfate, filtered, and concentrated under vacuum to afford 2-(2-{[(tert-butoxy)carbonyl](methyl)amino}ethoxy)-2-(3-chloro-4,5-difluorophenyl)acetic acid as a light yellow oil (180 mg, 93%). LCMS (ES, m/z): 380, 382 [M+H]⁺.

### Intermediate 42-1. 2-[3-(difluoromethoxy)phenyl]-2-(2-methoxyethoxy)acetic Acid

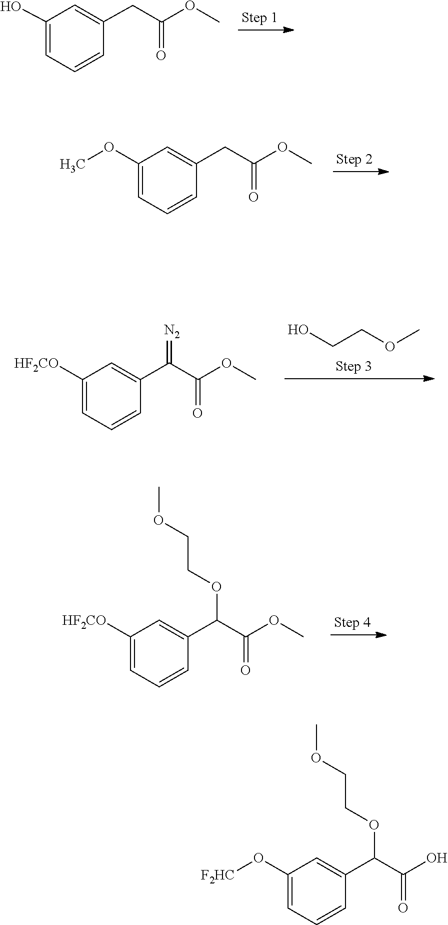

### Step 1. Methyl 2-[3-(difluoromethoxy)phenyl]acetate

To a solution of methyl 2-(3-hydroxyphenyl)acetate (2.00 g, 11.4 mmol) in DMF (20 mL) was added sodium 2-chloro-2,2-difluoroacetate (2.75 g, 17.1 mmol) and cesium carbonate (7.84 g, 22.9 mmol). The resulting mixture was stirred for 12 h at 90° C. and cooled to room temperature. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 3:1 ethyl acetate/petroleum ether) to afford methyl 2-[3-(difluoromethoxy)phenyl]acetate as yellow oil (1.00 g, 34%). LCMS (ES, m/z): 217 [M+H]⁺.

### Step 2. Methyl 2-diazo-2-[3-(difluoromethoxy)phenyl]acetate

To a solution of methyl 2-[3-(difluoromethoxy)phenyl]acetate (1.00 g, 3.93 mol) in MeCN (15 mL) was added 1-(azidodimethylidene-lambda6-sulfanyl)-4-methylbenzene (979 mg, 4.72 mmol) and DBU (882 mg, 5.51 mmol) at 0° C. The resulting mixture was stirred for 12 h at rt. The reaction mixture was poured into water (10 mL) and then extracted with ethyl acetate (3×10 mL). The combined organic layers were washed with saturated ammonium chloride solution (10 mL) and then dried over anhydrous sodium sulfate, filtered, and concentrated under vacuum. The resulting crude product was purified by silica gel chromatography (eluting with 4:1 ethyl acetate/petroleum ether) to afford methyl 2-diazo-2-[3-(difluoromethoxy)phenyl]acetate as a yellow oil (800 mg, 71%). LCMS (ES, m/z): 243 [M+H]⁺.

### Step 3. Methyl 2-[3-(difluoromethoxy)phenyl]-2-(2-methoxyethoxy)acetate

To a solution of methyl 2-diazo-2-[3-(difluoromethoxy)phenyl]acetate (400 mg, 1.40 mmol) in DCM (5 mL) was added Rhodium (II) acetate dimer (26 mg, 0.06 mmol) and 2-methoxyethan-1-ol (135 mg, 1.69 mmol). The resulting mixture was stirred for 4 h at rt. The resulting mixture was filtered and concentrated under vacuum. The residue was purified by silica gel column chromatography (eluted with 1:3 EtOAc/petroleum ether) to afford methyl 2-[3-(difluoromethoxy)phenyl]-2-(2-methoxyethoxy)acetate as a yellow oil (300 mg, 63%). LCMS (ES, m/z): 291 [M+H]⁺.

### Step 4. 2-[3-(difluoromethoxy)phenyl]-2-(2-methoxyethoxy)acetic Acid

To a solution of methyl 2-[3-(difluoromethoxy)phenyl]-2-(2-methoxyethoxy)acetate (300 mg, 0.88 mmol) in THF (3 mL) was added water (3 mL) and lithium hydroxide (111 mg, 4.39 mmol). The resulting mixture was stirred for 16 h at rt. The resulting mixture was washed with diethyl ether (1×5 mL) and acidified to pH=5 with hydrochloric acid (0.5 N). The resulting solution was extracted with ethyl acetate (3×5 mL). The combined organic layers were dried over anhydrous sodium sulfate, filtered, and concentrated under vacuum to afford 2-[3-(difluoromethoxy)phenyl]-2-(2-methoxyethoxy)acetic acid as a white solid (150 mg, 53%). LCMS (ES, m/z): 277 [M+H]⁺.

### Intermediate 43-1. 2-hydroxy-2-(2-methyl-1,3-benzoxazol-4-yl)acetic Acid

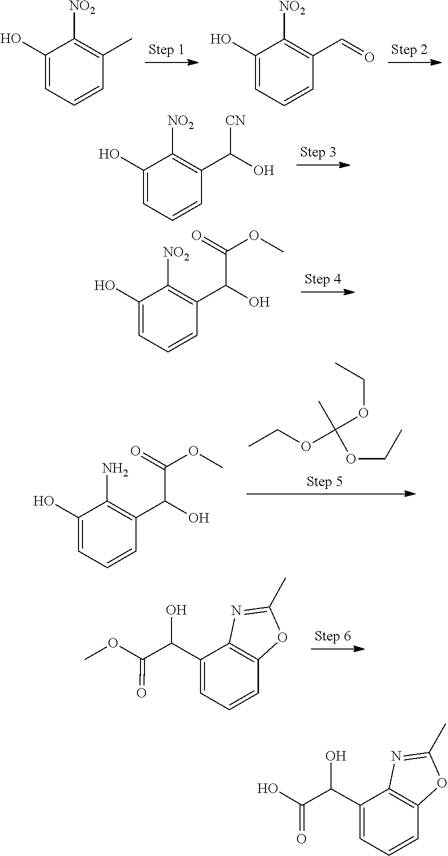

### Step 1. 3-hydroxy-2-nitrobenzaldehyde

To a solution of 3-methyl-2-nitrophenol (200 g, 1.29 mol) in acetic anhydride (1600 mL) was added sulfuric acid (240 mL) and acetic acid (1620 mL). This was followed by the addition of chromium trioxide (280 g, 2.77 mol) in several batches with stirring at 0° C. The resulting mixture was stirred for 2.5 h at 0° C. and then poured into ice/water (5000 mL). The solids were collected by filtration and then washed with water (3×1 L), saturated sodium carbonate solution (3×800 mL), and water (3×1 L). The solids were dissolved in ethanol (380 mL) and concentrated hydrochloric acid (617 mL). The resulting solution was stirred for 1.5 h at 110° C. and then cooled to room temperature. The reaction mixture was concentrated under vacuum to afford 3-hydroxy-2-nitrobenzaldehyde as a yellow solid (38.0 g, 18%). LCMS (ES, m/z): 166 [M−H]⁻.

### Step 2. 2-hydroxy-2-(3-hydroxy-2-nitrophenyl)acetonitrile

To a solution of 3-hydroxy-2-nitrobenzaldehyde (38.0 g, 204 mmol) in dichloromethane (500 mL) was added ZnI₂ (14.5 g, 44.5 mmol). The reaction was treated with trimethylsilyl cyanide (100 mL, 708 mmol) added dropwise with stirring at 0° C. The resulting mixture was stirred for 2.5 h at 25° C. The reaction was poured into brine (200 mL) and then extracted with ethyl acetate (3×500 mL). The combined organic layers were dried over anhydrous sodium sulfate, filtered, and concentrated under vacuum to afford 2-hydroxy-2-(3-hydroxy-2-nitrophenyl)acetonitrile as a yellow solid (34.0 g, 73%). LCMS (ES, m/z): 195 [M+H]⁺.

### Step 3. Methyl 2-hydroxy-2-(3-hydroxy-2-nitrophenyl)acetate

To a solution of 2-hydroxy-2-(3-hydroxy-2-nitrophenyl)acetonitrile (34.0 g, 157 mmol) in methanol (80 mL) was added hydrochloric acid (80 mL, 4 N in 1,4-dioxane). The resulting solution was stirred for 45 min at 60° C. and cooled to room temperature. The reaction mixture was concentrated under vacuum and purified by silica gel chromatography (eluting with 0:100 to 35:65 ethyl acetate/petroleum ether) to afford methyl 2-hydroxy-2-(3-hydroxy-2-nitrophenyl)acetate as a yellow solid (23.0 g, 58%). LCMS (ES, m/z): 228 [M+H]⁺.

### Step 4. Methyl 2-(2-amino-3-hydroxyphenyl)-2-hydroxyacetate

To a solution of methyl 2-hydroxy-2-(3-hydroxy-2-nitrophenyl)acetate (23.0 g, 0.11 mol) in methanol (500 mL) was added anhydrous palladium carbon (2.3 g, 10 wt % Pd). The resulting mixture was stirred for 16 h at 25° C. under hydrogen atmosphere (3 atm). The reaction mixture was filtered and concentrated under vacuum to afford methyl 2-(2-amino-3-hydroxyphenyl)-2-hydroxyacetate as a yellow solid (14.0 g, 60%). LCMS (ES, m/z): 198 [M+H]⁺.

### Step 5. Methyl 2-hydroxy-2-(2-methyl-1,3-benzoxazol-4-yl)acetate

To a solution of methyl 2-(2-amino-3-hydroxyphenyl)-2-hydroxyacetate (9.0 g, 43.4 mmol) in 1,1,1-triethoxyethane (150 mL) was added bismuth (III) trifluoromethanesulfonate (800 mg, 1.18 mmol). The resulting mixture was stirred for 10 min at 85° C. After cooling to room temperature, the reaction mixture was concentrated under vacuum. The resulting crude product was purified by silica gel chromatography (eluting with 0:100 to 50:50 ethyl acetate/petroleum ether) to afford methyl 2-hydroxy-2-(2-methyl-1,3-benzoxazol-4-yl)acetate as a white solid (6.3 g, 63%). LCMS (ES, m/z): 222 [M+H]⁺.

### Step 6. 2-hydroxy-2-(2-methyl-1,3-benzoxazol-4-yl)acetic Acid

To a solution of methyl 2-hydroxy-2-(2-methyl-1,3-benzoxazol-4-yl)acetate (500 mg, 2.26 mmol) in tetrahydrofuran (20 mL) and water (2 mL) was added lithium hydroxide (271 mg, 11.3 mmol). The resulting mixture was stirred for 1 h at room temperature. The reaction mixture was washed with diethyl ether (1×10 mL) and then acidified to pH=6 with hydrochloric acid (1 N). The resulting solution was extracted with ethyl acetate (2×20 mL). The combined organic layers were dried over anhydrous sodium sulfate, filtered, and concentrated under vacuum to afford 2-hydroxy-2-(2-methyl-1,3-benzoxazol-4-yl)acetic acid as a white solid (386 mg, 82%). LCMS (ES, m/z): 208 [M+H]⁺.

### Intermediate 44-1. 2-methyl-1,3-benzothiazole-4-carbaldehyde

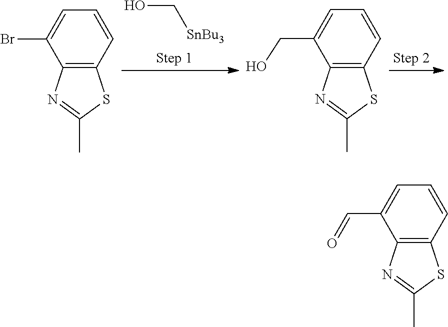

### Step 1. (2-methyl-1,3-benzothiazol-4-yl)methanol

To a solution of 4-bromo-2-methyl-1,3-benzothiazole (2.10 g, 9.21 mmol) in 1,4-dioxane (70 mL) was added (tributylstannyl)methanol (3.84 g, 12.0 mmol), and Pd(PPh₃)₄(1.60 g, 1.38 mmol). The resulting mixture was stirred for 16 h at 100° C. and then cooled to room temperature. The reaction mixture was poured into water (50 mL) and then extracted with ethyl acetate (3×70 mL). The combined organic layers were dried over anhydrous sodium sulfate, filtered, and concentrated under vacuum. The crude product was purified by silica gel chromatography (eluting with 0:100 to 80:20 ethyl acetate/petroleum ether) to afford (2-methyl-1,3-benzothiazol-4-yl)methanol as a yellow oil (1.20 g, 73%). LCMS (ES, m/z): 180 [M+H]⁺.

### Step 2. 2-methyl-1,3-benzothiazole-4-carbaldehyde

To a solution of oxalyl chloride (1.39 mL, 13.39 mmol) in dichloromethane (30 mL) was added DMSO (1.43 mL, 20.1 mmol) dropwise with stirring at −78° C. The resulting mixture was stirred for 30 min at −78° C. The reaction was treated with (2-methyl-1,3-benzothiazol-4-yl)methanol (1.20 g, 6.69 mmol) in dichloromethane (10 mL) added slowly at −78° C. After 2 h TEA (5.58 mL, 40.1 mmol) was added and the mixture was warmed to room temperature and stirred for 2 h. The reaction was poured into brine (30 mL) and then extracted with ethyl acetate (3×50 mL). The combined organic layers were dried over anhydrous sodium sulfate, filtered, and concentrated under vacuum to afford 2-methyl-1,3-benzothiazole-4-carbaldehyde as a yellow oil (900 mg, 76%). LCMS (ES, m/z): 178 [M+H]⁺.

### Intermediate 45-1. 2-hydroxy-2-(2-methyl-1,3-benzothiazol-4-yl)acetic Acid

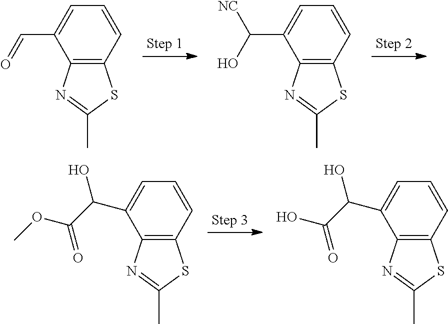

### Step 1. 2-hydroxy-2-(2-methyl-1,3-benzothiazol-4-yl)acetonitrile

To a solution of 2-methyl-1,3-benzothiazole-4-carbaldehyde (900 mg, 5.08 mmol) in DCM (10 mL) was added trimethylsilyl cyanide (2.02 mL, 15.2 mmol) and ZnI₂ (162 mg, 0.51 mmol). The resulting mixture was stirred for 1 h at room temperature. The reaction mixture was poured into water (5 mL) and then extracted with ethyl acetate (3×10 mL). The combined organic layers were dried over anhydrous sodium sulfate, filtered, and concentrated under vacuum to afford 2-hydroxy-2-(2-methyl-1,3-benzothiazol-4-yl)acetonitrile as yellow oil (800 mg, 65%). LCMS (ES, m/z): 205 [M+H]⁺.

### Step 2. methyl 2-hydroxy-2-(2-methyl-1,3-benzothiazol-4-yl)acetate

To a solution of 2-hydroxy-2-(2-methyl-1,3-benzothiazol-4-yl)acetonitrile (800 mg, 3.92 mmol) in methanol (30 mL) was added concentrated hydrochloric acid (30 mL, 12 N). The resulting solution was stirred for 4 h at 60° C. and cooled to room temperature. The reaction mixture was poured into water (5 mL) and then extracted with ethyl acetate (3×10 mL). The combined organic layers were dried over anhydrous sodium sulfate, filtered, and concentrated under vacuum to afford methyl 2-hydroxy-2-(2-methyl-1,3-benzothiazol-4-yl)acetate as a yellow oil (300 mg, crude). LCMS (ES, m/z): 238 [M+H]V.

### Step 3. 2-hydroxy-2-(2-methyl-1,3-benzothiazol-4-yl)acetic Acid

To a solution of 2-hydroxy-2-(2-methyl-1,3-benzothiazol-4-yl)acetic acetate (500 mg, 2.24 mmol) in tetrahydrofuran (3 mL) and water (3 mL) was added lithium hydroxide (253 mg, 10.6 mmol). The resulting mixture was stirred for 16 h at room temperature. The reaction mixture was washed with diethyl ether (1×8 mL) and then acidified to pH=5 with saturated citric acid. The resulting solution was extracted with ethyl acetate (2×8 mL). The organic layers were combined, dried over anhydrous sodium sulfate, filtered, and concentrated under vacuum to afford 2-hydroxy-2-(2-methyl-1,3-benzothiazol-4-yl)acetic acid as yellow oil (300 mg, 58%). LCMS (ES, m/z): 224 [M+H]⁺.

The Intermediates in Table 12 were synthesized according to the procedure described for Intermediate 45-2 above.

TABLE 12

| Inter- | | LCMS: (ESI) |
|---|---|---|
| mediate | Structure and Name | m/z [M + H]⁺ |
| 45-2 | 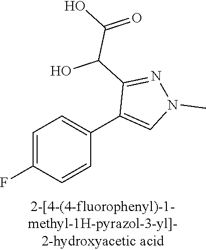 [SMILES: Cn1cc(-c2ccc(F)cc2)c(C(O)C(=O)O)n1] | 251 |
| 45-3 | 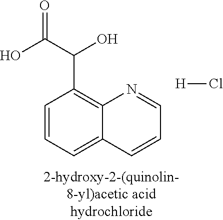 | 240 |
| 45-4 | 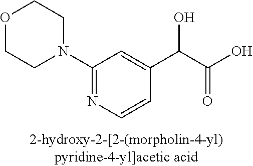 [SMILES: O=C(O)C(O)c1ccnc(N2CCOCC2)c1] | 239 |

### Intermediate 46-1. 4-(4-fluorophenyl)-1-methyl-1H-pyrazole-3-carbaldehyde

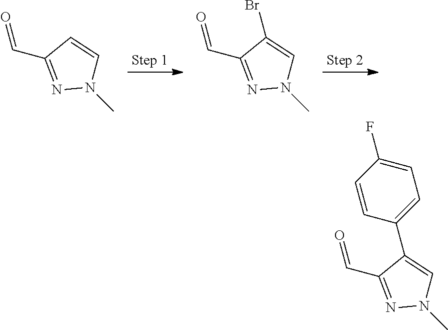

### Step 1. 4-bromo-1-methyl-1H-pyrazole-3-carbaldehyde

To a solution of 1-methyl-1H-pyrazole-3-carbaldehyde (10.0 g, 89.0 mmol) in N,N-dimethylformamide (120 mL), was added a solution of NBS (16.0 g, 88.1 mmol) in N,N-dimethylformamide (30 mL) with stirring at 0° C. The solution was stirred for 2 h at 25° C. The reaction mixture was poured into water (300 mL) and treated with saturated sodium hydroxide solution (15 mL). The product was extracted with ethyl acetate (3×150 mL). The combined organic layers were dried over anhydrous sodium sulfate, filtered, and concentrated under vacuum. The crude product was purified by silica gel chromatography (eluting with 0:100 to 50:50 ethyl acetate/petroleum ether) to afford 4-bromo-1-methyl-1H-pyrazole-3-carbaldehyde as a white solid (13.0 g, 77%). LCMS (ES, m/z): 189, 191 [M+H]⁺.

### Step 2. 4-(4-fluorophenyl)-1-methyl-1H-pyrazole-3-carbaldehyde

To a solution of 4-bromo-1-methyl-1H-pyrazole-3-carbaldehyde (1.0 g, 5.18 mmol) in 1,4-dioxane (20 mL), was added (4-fluorophenyl)boronic acid (894 mg, 6.26 mmol), cesium carbonate (3.5 g, 10.7 mmol), Pd(dppf)Cl₂.CH₂Cl₂ (435 mg, 0.53 mmol) and water (5 mL). The resulting mixture was stirred for 3 h at 90° C. and cooled to room temperature. The reaction mixture was poured into water (50 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 4-(4-fluorophenyl)-1-methyl-1H-pyrazole-3-carbaldehyde as a light brown solid (900 mg, 85%). LCMS (ES, m/z): 205 [M+H]⁺.

### Intermediate 47-1. 3,4-dihydro-2H-1-benzopyran-7-sulfonyl Chloride

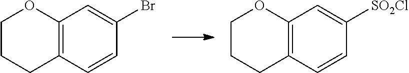 [SMILES: Brc1ccc2c(c1)OCCC2 ; O=S(=O)(Cl)c1ccc2c(c1)OCCC2]

To a solution of n-BuLi (8.5 mL, 2.5 M in heptane) was added n-Bu₂Mg (21 mL, 1.0 M in hexane). The resulting mixture was stirred for 30 min at room temperature. This was followed by the dropwise addition of a solution of 7-bromo-3,4-dihydro-2H-1-benzopyran (3.0 g, 13.8 mmol) in tetrahydrofuran (25 mL) dropwise with stirring at −10° C. The resulting mixture was stirred for 2 h at −10° C. and was then slowly added to a solution of sulfuryl dichloride (25 mL) in toluene (25 mL) at −10° C. The resulting mixture was stirred for 30 min at −10° C. The reaction was then quenched by the careful addition saturated aqueous ammonium chloride solution (100 mL) at 0° C. The resulting solution was extracted with ethyl acetate (3×100 mL). The organic layers were combined, dried over anhydrous sodium sulfate, filtered and concentrated under vacuum. The resulting crude product was purified by silica gel column (eluting with 0:100 to 30:70 ethyl acetate/petroleum ether) to afford 3,4-dihydro-2H-1-benzopyran-7-sulfonyl chloride as a yellow oil (2.5 g, 78%). GCMS (ES): 232 [M]⁺.

### Intermediate 48-1. 4-bromo-3-methyl-1,2-benzoxazole

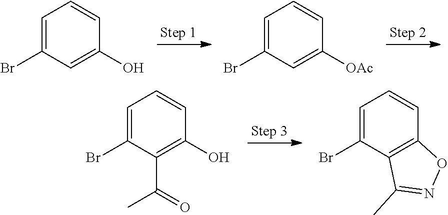

### Step 1. 3-bromophenyl Acetate

To a solution of 3-bromophenol (10 g, 57.2 mmol) in acetic anhydride (17.4 mL, 184 mmol) was added sulfuric acid (0.2 mL). The resulting mixture was stirred for 18 h at 25° C. The reaction mixture was poured into hydrochloric acid (100 mL, 2 wt %) and then extracted with ethyl acetate (3×100 mL). The combined organic layers were washed with sodium bicarbonate solution (2 N) twice and brine. The solution was dried over anhydrous sodium sulfate, filtered and concentrated under vacuum to afford 3-bromophenyl acetate as a colorless oil (13 g, crude). LCMS (ES, m/z): 215, 217 [M+H]⁺.

### Step 2. 1-(2-bromo-6-hydroxyphenyl)ethan-1-one

To 3-bromophenyl acetate (13 g, 56.2 mmol) was added trichloroaluminum (12 g, 90.0 mmol). The resulting mixture was stirred for 2 h at 145° C. After cooling to room temperature, the reaction mixture was poured into hydrochloric acid (32 mL, 5 wt %) and then extracted with DCM (3×50 mL). The combined organic layers were washed with sodium hydroxide (100 mL, 5 N). The aqueous layer was acidified to pH=2 with concentrated hydrochloric acid and then extracted with ethyl acetate (5×50 mL). The combined organic layers were dried over anhydrous sodium sulfate, filtered and concentrated under vacuum to afford 1-(2-bromo-6-hydroxyphenyl)ethan-1-one as a white solid (7.4 g, 61%). LCMS (ES, m/z): 215, 217 [M+H]⁺.

### Step 3. 4-bromo-3-methyl-1,2-benzoxazole

To 1-(2-bromo-6-hydroxyphenyl)ethan-1-one (3 g, 13.0 mmol) was added ammonia solution (15 mL, 7 M in methanol). The resulting mixture was stirred for 3 h at 25° C. The solids were collected by filtration, washed with diethyl ether and then dissolved with THF (20 mL). The reaction was treated with N-Chlorosuccinimide (2.1 g, 15.7 mmol) and potassium carbonate (2.85 g, 20.5 mmol). The resulting mixture was stirred for 18 h at 25° C. The reaction mixture was poured into water (50 mL) and then extracted with ethyl acetate (3×50 mL). The combined organic layers were dried over anhydrous sodium sulfate, filtered and concentrated under vacuum. The resulting crude product was purified by silica gel chromatography (eluting with 0:100 to 20/80 ethyl acetate/petroleum ether) to afford 4-bromo-3-methyl-1,2-benzoxazole as a yellow solid (1.8 g, 65%). LCMS (ES, m/z): 212, 214 [M+H]⁺.

### Intermediate 49-1. 2-(morpholin-4-yl)pyridine-4-carbaldehyde

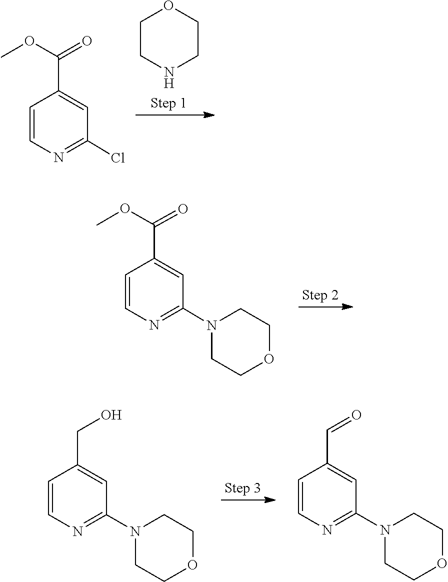 [SMILES: COC(=O)c1ccnc(Cl)c1 ; C1COCCN1 ; COC(=O)c1ccnc(N2CCOCC2)c1 ; OCc1ccnc(N2CCOCC2)c1 ; O=Cc1ccnc(N2CCOCC2)c1]

### Step 1. Methyl 2-(morpholin-4-yl)pyridine-4-carboxylate

To a solution of morpholine (1.53 g, 17.2 mmol) in DMF (30 mL), was added methyl 2-chloropyridine-4-carboxylate (2.00 g, 11.4 mmol), and potassium carbonate (3.20 g, 22.7 mmol). The resulting mixture was stirred for 18 h at 100° C. and then cooled to room temperature. The reaction mixture was poured into water (30 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford methyl 2-(morpholin-4-yl)pyridine-4-carboxylate as a yellow oil (375 mg, 15%). LCMS (ES, m/z): 223 [M+H]⁺.

### Step 2. [2-(morpholin-4-yl)pyridine-4-yl]methanol

To a solution of methyl 2-(morpholin-4-yl)pyridine-4-carboxylate (375 mg, 1.65 mmol) in tetrahydrofuran (3 mL), was added lithium aluminium hydride (2.1 mL, 1M in THF) dropwise at −10° C. The resulting mixture was stirred for 15 min at −10° C. and quenched by careful addition of Na₂SO₄.10H₂O (500 mg). The resulting mixture was filtered and concentrated under vacuum to afford [2-(morpholin-4-yl)pyridin-4-yl]methanol as a yellow oil (210 mg, 65%). LCMS (ES, m/z): 195 [M+H]⁺.

### Step 3. 2-(morpholin-4-yl)pyridine-4-carbaldehyde

To a solution of [2-(morpholin-4-yl)pyridin-4-yl]methanol (150 mg, 0.77 mmol) in dichloromethane (2 mL), was added DMP (120 mg, 0.85 mmol) at 0° C. The resulting mixture was stirred for 18 h at room temperature. The reaction mixture was poured into water (2 mL) and then extracted with ethyl acetate (3×3 mL). The combined organic layers were dried over anhydrous sodium sulfate, filtered, and concentrated under vacuum to afford 2-(morpholin-4-yl)pyridine-4-carbaldehyde as a yellow oil (130 mg, 88%). LCMS (ES, m/z): 193 [M+H]⁺.

### Intermediate 50-1. Methyl 2-(1,2-benzoxazol-3-yl)-2-hydroxyacetate

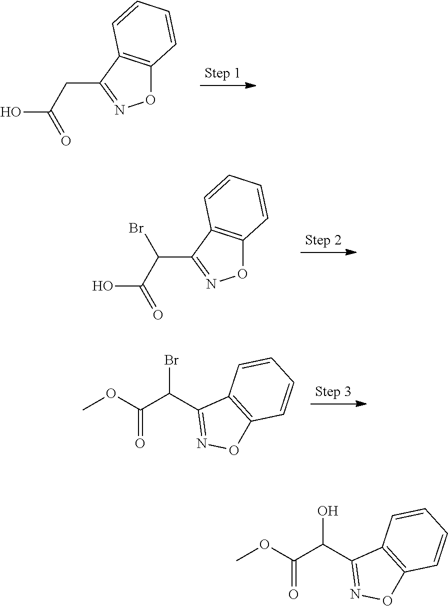

### Step 1. Ethyl 2-[2-methylimidazo[1,2-a]pyridin-8-yl]-2-oxoacetate

To a solution of 2-(1,2-benzoxazol-3-yl)acetic acid (2.50 g, 14.1 mmol) in acetic acid (25 mL) was added bromine (0.74 mL, 14.4 mmol) at 0° C. The resulting mixture was stirred for 16 h at room temperature. The reaction mixture was concentrated under vacuum. The resulting solid was washed with DCM and dried under vacuum to afford 2-(1,2-benzoxazol-3-yl)-2-bromoacetic acid as a light yellow solid (2.60 g, 72%). LCMS (ES, m/z): 256, 258 [M+H]⁺.

### Step 2. Methyl 2-(1,2-benzoxazol-3-yl)-2-bromoacetate

To a solution of 2-(1,2-benzoxazol-3-yl)-2-bromoacetic acid (2.60 g, 10.2 mmol) in diethyl ether (30 mL) was added thionyl chloride (3.66 mL, 50.4 mmol). The resulting solution was stirred for 15 min at 40° C. Then the resulting mixture was concentrated under vacuum and then dissolved with methanol (30 mL). The resulting solution was stirred for 16 h at room temperature. The reaction mixture was poured into brine (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 15:85 ethyl acetate/petroleum ether) to afford methyl 2-(1,2-benzoxazol-3-yl)-2-bromoacetate as a light yellow oil (2.00 g, 73%). LCMS (ES, m/z): 270, 272 [M+H]⁺.

### Step 3. Methyl 2-(1,2-benzoxazol-3-yl)-2-hydroxyacetate

To a solution of methyl 2-(1,2-benzoxazol-3-yl)-2-bromoacetate (2.00 g, 7.41 mmol) in methanol (30 mL) and water (15 mL) was added sodium nitrite (2.30 g, 33.3 mmol). The resulting mixture was stirred for 3 days at room temperature. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford methyl 2-(1,2-benzoxazol-3-yl)-2-hydroxyacetate as a light yellow oil (180 mg, 12%). LCMS (ES, m/z): 208 [M+H]⁺.

The Intermediates in Table 13 were synthesized according to the procedure described for Intermediate 50-1 above.

TABLE 13

| | | LCMS: (ESI) |
|---|---|---|
| Intermediate | Structure and Name | m/z [M + H]⁺ |
| 50-2 | 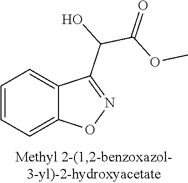 | 208 |

### Intermediate 51-1. 2-hydroxy-2-(1-methyl-1H-indol-2-yl)acetic Acid

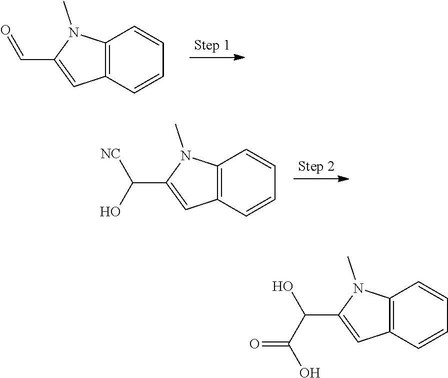

### Step 1. 2-hydroxy-2-(1-methyl-1H-indol-2-yl)acetonitrile

To a solution of 1-methyl-1H-indole-2-carbaldehyde (2.0 g, 12.4 mmol) in dichloromethane (40 mL) was added ZnI₂ (800 mg, 2.51 mmol). This was followed by the addition of trimethylsilyl cyanide (4.97 mL, 37.3 mmol) with stirring at 0° C. The resulting solution was stirred for 2 h at 0° C. and then concentrated under vacuum to afford 2-hydroxy-2-(1-methyl-1H-indol-2-yl)acetonitrile as yellow oil (2.0 g, 86%). LCMS (ES, m/z): 187 [M+H]⁺.

### Step 2. 2-hydroxy-2-(1-methyl-1H-indol-2-yl)acetic Acid

To a solution of 2-hydroxy-2-(1-methyl-1H-indol-2-yl)acetonitrile (2.0 g, 9.67 mmol) in 1,4-dioxane (10 mL) was added aqueous sodium tetraborate decahydrate solution (60 mL, 0.5 M). The reaction mixture was stirred for 2 h at 80° C. The reaction was treated with aqueous sodium hydroxide (60 mL, 1.0 M) and stirred for 3 h at 100° C. Upon cooling to room temperature, the solution was acidified to pH=8 with concentrated hydrochloric acid. The resulting mixture was lyophilized. The resulting crude product was purified by silica gel chromatography (eluting with 0:100 to 10:90 dichloromethane/methanol) to afford 2-hydroxy-2-(1-methyl-1H-indol-2-yl) acetic acid as a yellow solid (200 mg, 10%). LCMS (ES, m/z): 206 [M+H]⁺.

### Intermediate 52-1. 5-fluoro-2,3-dihydro-1,4-benzodioxine-6-sulfonyl Chloride

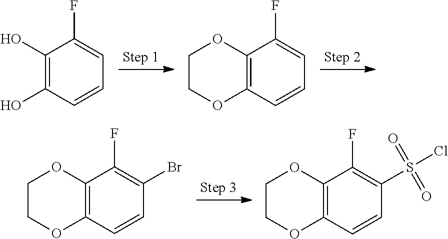

### Step 1. 5-fluoro-2,3-dihydrobenzo[b][1,4]dioxine

To a solution of 3-fluorobenzene-1,2-diol (10.0 g, 76.5 mmol) in N,N-dimethylformamide (150 mL) was added 1,2-dibromoethane (18.9 g, 98.6 mmol) and cesium carbonate (76.0 g, 233 mmol). The resulting mixture was stirred at 16 h at 120° C. and then cooled to room temperature. The resulting solution was poured into water (500 mL) and then extracted with ethyl acetate (3×150 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 5-fluoro-2,3-dihydrobenzo[b][1,4]dioxine as a yellow oil (5 g, 43%). LCMS (ES, m/z): 155 [M+H]⁺.

### Step 2. 6-bromo-5-fluoro-2,3-dihydro-1,4-benzodioxine

To a solution of 5-fluoro-2,3-dihydro-1,4-benzodioxine (3.00 g, 18.5 mmol) in methanol (115 mL) was added bromine (1.2 mL, 23.0 mmol). The resulting solution was stirred for 18 h at room temperature. The reaction mixture was poured into aqueous sodium hydrogen sulfite solution (50 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 6-bromo-5-fluoro-2,3-dihydro-1,4-benzodioxine as a yellow oil (2.00 g, 44%). LCMS (ES, m/z): 233, 235 [M+H]⁺.

### Step 3. 5-fluoro-2,3-dihydro-1,4-benzodioxine-6-sulfonyl Chloride

To n-Bu₂Mg (13 mL, 1.0 M in hexane) was added n-BuLi (15 mL, 2.5 M in heptane). After stirring for 5 min at room temperature, the reaction was cooled to −15° C. and treated with a 6-bromo-5-fluoro-2,3-dihydrobenzo[b][1,4]dioxine (2.00 g, 8.15 mmol) in tetrahydrofuran (15 mL) at −15° C. The resulting mixture was stirred for 1 h at −15° C. and then a solution of sulfuryl dichloride (15 mL) in toluene (15 mL) was added. The resulting mixture was warmed to −10° C. and stirred for 0.5 h. The reaction mixture was poured into saturated ammonium chloride solution (50 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford 5-fluoro-2,3-dihydro-1,4-benzodioxine-6-sulfonyl chloride as yellow oil (200 mg, 10%). LCMS (ES, m/z): 253, 255 [M+H]⁺.

### Intermediate 53-1. 2-(5-fluoro-2,3-dihydro-1,4-benzodioxine-6-sulfonyl)-1H,2H,3H,4H,5H,6H-pyrrolo[3,4-c]pyrrole

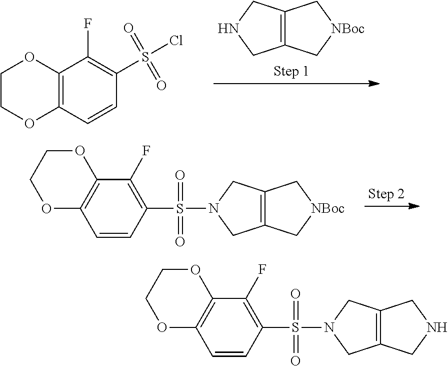

### Step 1. 2-tert-butyl-7-(5-fluoro-2,3-dihydro-1,4-benzodioxine-6-sulfonyl)-1H,3H,4H,5H,6H,7H,8H-2lambda3,4,7-pyrrolo[3,4-e][1lambda3,3]oxazepin-3-one

To a solution of 5-fluoro-2,3-dihydro-1,4-benzodioxine-6-sulfonyl chloride (200 mg, 0.71 mmol) in dichloromethane (5 mL) was added 2-tert-butyl-1H,3H,4H,5H,6H,7H,8H-2lambda3,4,7-pyrrolo[3,4-e][1lambda3,3]oxazepin-3-one (167 mg, 0.75 mmol) and DIEA (0.26 mL, 1.55 mmol). The resulting solution was stirred for 2 h at 25° C. The reaction mixture was poured into water (15 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 2-tert-butyl-7-(5-fluoro-2,3-dihydro-1,4-benzodioxine-6-sulfonyl)-1H,3H,4H,5H,6H,7H,8H-2lambda3,4,7-pyrrolo[3,4-e][1lambda3,3]oxazepin-3-one as colorless oil (150 mg, 49%). LCMS (ES, m/z): 427 [M+H]⁺.

### Step 2. 2-(5-fluoro-2,3-dihydro-1,4-benzodioxine-6-sulfonyl)-1H,2H,3H,4H,5H,6H-pyrrolo[3,4-c]pyrrole

To a solution of 2-tert-butyl-7-(5-fluoro-2,3-dihydro-1,4-benzodioxine-6-sulfonyl)-1H,3H,4H,5H,6H,7H,8H-2lambda3,4,7-pyrrolo[3,4-e][1lambda3,3]oxazepin-3-one (70 mg, 0.16 mmol) in dichloromethane (5 mL) was added trifluoroacetic acid (1 mL). The resulting solution was stirred for 1 h at room temperature. The resulting mixture was concentrated under vacuum and then basified to pH=8 with saturated potassium carbonate solution. The resulting solution was extracted with dichloromethane (2×5 mL). The organic layers were combined, dried over anhydrous sodium sulfate, filtered, and concentrated under vacuum to afford 2-(5-fluoro-2,3-dihydro-1,4-benzodioxine-6-sulfonyl)-1H,2H,3H,4H,5H,6H-pyrrolo[3,4-c]pyrrole as brown oil (40 mg, 76%). LCMS (ES, m/z): 327 [M+H]⁺.

### Intermediate 54-1. 2-hydroxy-2-(3-iodophenyl)acetic Acid

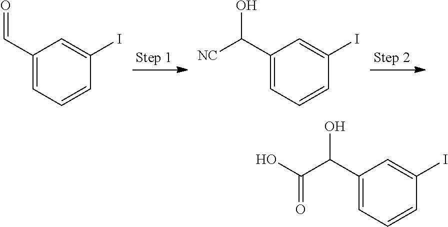

### Step 1. 2-hydroxy-2-(3-iodophenyl)acetonitrile

To a solution of 3-iodobenzaldehyde (1.00 g, 4.22 mmol) in DCM (30 mL), was added ZnI₂ (274 mg, 0.84 mmol) and trimethylsilyl cyanide (1.72 mL, 12.6 mmol) added dropwise with stirring at 0° C. for 3 h. The reaction was poured into water (20 mL) and then extracted with ethyl acetate (3×100 mL). The combined organic layers were dried over anhydrous sodium sulfate, filtered and concentrated under vacuum to afford 2-hydroxy-2-(3-iodophenyl)acetonitrile as yellow oil (0.8 g, 73%). LCMS (ES, m/z): 260 [M+H]⁺.

### Step 2. 2-hydroxy-2-(3-iodophenyl)acetic Acid

To a solution of 2-hydroxy-2-(3-iodophenyl)acetonitrile (1.47 g, 4.54 mmol) in 1,4-dioxane (8 mL) was added concentrated hydrochloric acid (3.5 mL). The resulting mixture was stirred for 3 h at 95° C. After cooling to room temperature, the reaction mixture was diluted with water (5 mL) and then extracted with ethyl acetate (3×20 mL). The organic layers were combined, dried over anhydrous sodium sulfate, filtered and concentrated under vacuum to afford 2-hydroxy-2-(3-iodophenyl)acetic acid as a yellow oil (750 mg, 59%). LCMS (ES, m/z): 279 [M+H]⁺.

The Intermediates in Table 14 were synthesized according to the procedure described for Intermediate 54-1 above.

TABLE 14

| Inter- | | LCMS: |
|---|---|---|
| me- | | (ESI) m/z |
| diate | Structure and Name | [M + H]⁺ |
| 54-2 | 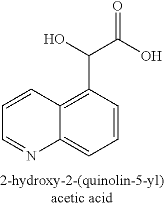 [SMILES: O=C(O)C(O)c1cccc2ncccc12] | 204 |
| 54-3 | 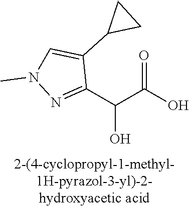 | 197 |
| 54-4 | 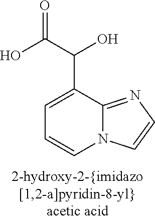 [SMILES: O=C(O)C(O)c1cccn2ccnc12] | 193 |
| 54-5 | 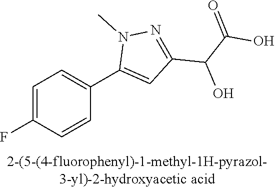 [SMILES: Cn1nc(C(O)C(=O)O)cc1-c1ccc(F)cc1] | 197 |
| 54-6 | 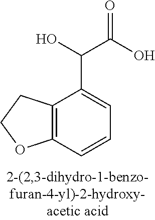 [SMILES: O=C(O)C(O)c1cccc2c1CCO2] | 195 |
| 54-7 | 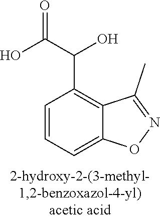 | 208 |
| 54-8 | 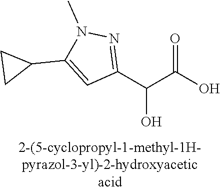 | 197 |
| 54-9 | 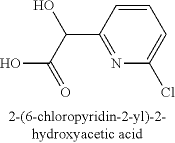 [SMILES: O=C(O)C(O)c1cccc(Cl)n1] | 188 |
| 54-10 | 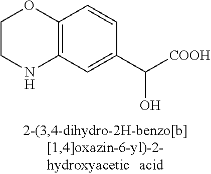 [SMILES: O=C(O)C(O)c1ccc2c(c1)NCCO2] | 210 |
| 54-11 | 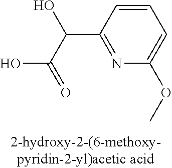 | 184 |
| 54-12 | 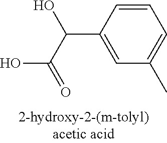 | 167 |
| 54-13 | 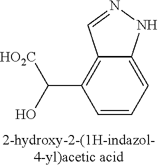 [SMILES: O=C(O)C(O)c1cccc2[nH]ncc12] | 193 |
| 54-14 | 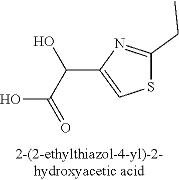 | 188 |

### Intermediate 55-1. 2-[(8-fluoro-2,3-dihydro-1,4-benzodioxin-6-yl)sulfonyl]-1H,2H,3H,4H,5H,6H-pyrrolo[3,4-c]pyrrole TFA Salt

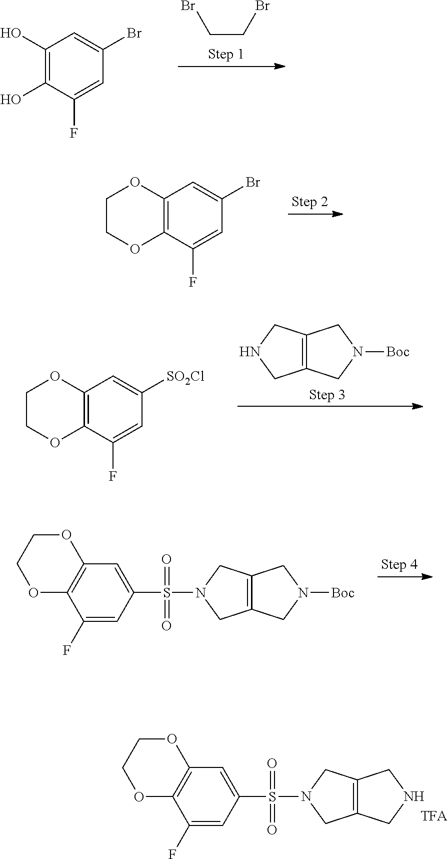

### Step 1. 7-bromo-5-fluoro-2,3-dihydro-1,4-benzodioxine

To a solution of 5-bromo-3-fluorobenzene-1,2-diol (5.00 g, 23.7 mmol) in N,N-dimethylformamide (80 mL) was added 1,2-dibromoethane (2.72 mL, 30.8 mmol), and cesium carbonate (24.0 g, 73.7 mmol). The resulting mixture was stirred for 16 h at 120° C. and then cooled to room temperature. The reaction mixture was poured into water (150 mL) and then extracted with ethyl acetate (3×150 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford 7-bromo-5-fluoro-2,3-dihydro-1,4-benzodioxine as a white solid (2.80 g, 51%). GCMS (EI, m/z): 233, 235 [M+H]⁺.

### Step 2. 8-fluoro-2,3-dihydro-1,4-benzodioxine-6-sulfonyl Chloride

To a stirred solution of n-BuLi (15 mL, 2.5 M in heptane) was added n-Bu₂Mg (13 mL, 1.0 M in hexane). After 5 min, the reaction was cooled to −10° C. and treated with 7-bromo-5-fluoro-2,3-dihydro-1,4-benzodioxine (2.0 g, 8.15 mmol) in tetrahydrofuran (15 mL). The resulting mixture was warmed to room temperature and stirred for 1 h. To the reaction was added sulfonylchloride (15 mL) in toluene (15 mL) and stirred for 0.5 h at −10° C. The reaction mixture was poured into saturated aqueous ammonium chloride (50 mL) and extracted with ethyl acetate (3×40 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford 8-fluoro-2,3-dihydro-1,4-benzodioxine-6-sulfonyl chloride as an off-white solid (800 mg, 38%). LCMS (ES, m/z): 253, 255 [M+H]⁺.

### Step 3. Tert-butyl 5-[(8-fluoro-2,3-dihydro-1,4-benzodioxin-6-yl)sulfonyl]-1H,2H,3H,4H,5H,6H-pyrrolo[3,4-c]pyrrole-2-carboxylate

To a solution of methyl tert-butyl 1H,2H,3H,4H,5H,6H-pyrrolo[3,4-c]pyrrole-2-carboxylate (1.00 g, 4.52 mmol) in dichloromethane (10 mL) was added DIEA (1.97 mL, 11.9 mmol). The reaction was treated with 8-fluoro-2,3-dihydro-1,4-benzodioxine-6-sulfonyl chloride (1.00 g, 3.80 mmol) in DCM (5 mL) added dropwise with stirring. The resulting mixture was stirred for 3 h at room temperature and then poured into water (50 mL). The product was extracted with ethyl acetate (3×50 mL). The combined organic layers were dried over anhydrous sodium sulfate, filtered, and concentrated under vacuum. The crude product was purified by silica gel chromatography (eluting with 0:100 to 50:50 ethyl acetate/petroleum ether) to afford tert-butyl 5-[(8-fluoro-2,3-dihydro-1,4-benzodioxin-6-yl)sulfonyl]-1H,2H,3H,4H,5H,6H-pyrrolo[3,4-c]pyrrole-2-carboxylate as a white solid (1.20 g, 74%). LCMS (ES, m/z): 427 [M+H]⁺.

### Step 4. 2-[(8-fluoro-2,3-dihydro-1,4-benzodioxin-6-yl)sulfonyl]-1H,2H,3H,4H,5H,6H-pyrrolo[3,4-c]pyrrole TFA Salt

To a solution of tert-butyl 5-[(8-fluoro-2,3-dihydro-1,4-benzodioxin-6-yl)sulfonyl]-1H,2H,3H,4H,5H,6H-pyrrolo[3,4-c]pyrrole-2-carboxylate (500 mg, 1.15 mmol) in dichloromethane (5 mL) was added trifluoroacetic acid (1 mL). The resulting solution was stirred for 1 h at room temperature and concentrated under vacuum to afford 2-[(8-fluoro-2,3-dihydro-1,4-benzodioxin-6-yl)sulfonyl]-1H,2H,3H,4H,5H,6H-pyrrolo[3,4-c]pyrrole TFA salt as a brown solid (400 mg, crude). LCMS (ES, m/z): 327 [M+H]⁺.

### Intermediate 56-1. Lithium 2-hydroxy-2-(4-methyl-3,4-dihydro-2H-1,4-benzoxazin-6-yl)acetate

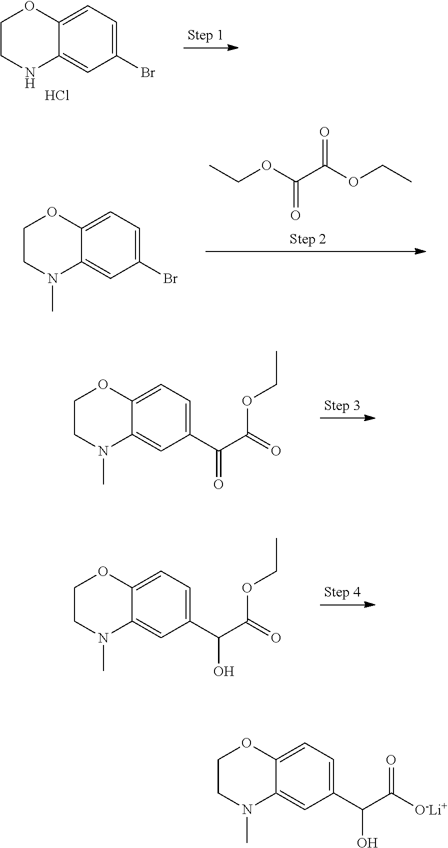

### Step 1. 6-bromo-4-methyl-3,4-dihydro-2H-1,4-benzoxazine

To a solution of 6-bromo-3,4-dihydro-2H-1,4-benzoxazine (5.00 g, 20.1 mmol) in CH₃CN (150 mL) was added paraformaldehyde (3.26 g, 40.2 mmol) and sodium cyanoborohydride (2.30 g, 36.49 mmol). The resulting mixture was stirred for 15 min at 0° C. The reaction was treated with acetic acid (5 mL) and stirred for 16 h at room temperature. The reaction was quenched with water (50 mL) and then extracted with ethyl acetate (3×150 mL). The combined organic layers were dried over anhydrous sodium sulfate, filtered, and concentrated under vacuum. The resulting crude product was purified by silica gel chromatography (eluting with 0:100 to 80:20 ethyl acetate/petroleum ether) to afford 6-bromo-4-methyl-3,4-dihydro-2H-1,4-benzoxazine as a red oil (2.50 g, 55%). LCMS (ES, m/z): 228, 230 [M+H]⁺.

### Step 2. Ethyl 2-(4-methyl-3,4-dihydro-2H-1,4-benzoxazin-6-yl)-2-oxoacetate

To a solution of 6-bromo-4-methyl-3,4-dihydro-2H-1,4-benzoxazine (2.50 g, 11.0 mmol) in THF (25 mL) was added n-BuLi (13.2 mL, 2.5 M in n-hexane) dropwise with stirring at −78° C. After 15 min diethyl oxalate (4.46 mL, 33.0 mmol) was added and stirring continued for 2 h. The reaction was poured into saturated aqueous ammonium chloride (10 mL). The product was extracted with ethyl acetate (3×25 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford ethyl 2-(4-methyl-3,4-dihydro-2H-1,4-benzoxazin-6-yl)-2-oxoacetate as a yellow oil (500 mg, 18%). LCMS (ES, m/z): 250 [M+H]⁺.

### Step 3. Ethyl 2-hydroxy-2-(4-methyl-3,4-dihydro-2H-1,4-benzoxazin-6-yl)acetate

To a solution of ethyl 2-(4-methyl-3,4-dihydro-2H-1,4-benzoxazin-6-yl)-2-oxoacetate (250 mg, 1.00 mmol) in tetrahydrofuran (10 mL) was added sodium borohydride (57 mg, 1.51 mmol). The resulting mixture was stirred for 10 min at 0° C. and then poured into water (10 mL). The product was extracted with ethyl acetate (3×10 mL). The combined organic layers were dried over anhydrous sodium sulfate, filtered, and concentrated under vacuum. The crude product was purified by silica gel chromatography (eluting with 0:100 to 20:80 ethyl acetate/petroleum ether) to afford ethyl 2-hydroxy-2-(4-methyl-3,4-dihydro-2H-1,4-benzoxazin-6-yl)acetate as a colorless oil (180 mg, 71%). LCMS (ES, m/z): 252 [M+H]⁺.

### Step 4 Lithium 2-hydroxy-2-(4-methyl-3,4-dihydro-2H-1,4-benzoxazin-6-yl)acetate

To a solution of ethyl 2-hydroxy-2-(4-methyl-3,4-dihydro-2H-1,4-benzoxazin-6-yl)acetate (180 mg, 0.72 mmol) in tetrahydrofuran (2 mL) and water (2 mL) was added lithium hydroxide (87 mg, 3.63 mmol). The resulting mixture was stirred for 16 h at room temperature. The reaction mixture was washed with diethyl ether (1×8 mL) and then concentrated under vacuum to afford lithium 2-hydroxy-2-(4-methyl-3,4-dihydro-2H-1,4-benzoxazin-6-yl)acetate as a yellow oil (100 mg, 16%). LCMS (ES, m/z): 224 [M+H]⁺.

### Intermediate 57-1. 2-[(2,2,3,3-tetrafluoro-2,3-dihydro-1,4-benzodioxin-6-yl)sulfonyl]-1H,2H,3H,4H,5H,6H-pyrrolo[3,4-c]pyrrole TFA Salt

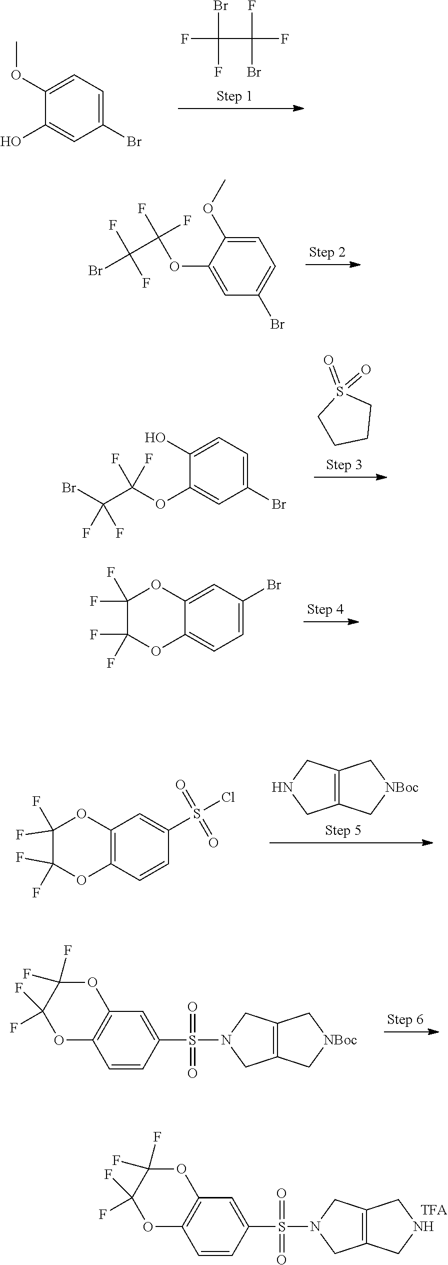

### Step 1. 4-bromo-2-(2-bromo-1,1,2,2-tetrafluoroethoxy)-1-methoxybenzene

To a solution of 5-bromo-2-methoxyphenol (10.0 g, 48.3 mmol) in DMSO (150 mL) was added 1,2-dibromo-1,1,2,2-tetrafluoroethane (26 g, 98.1 mmol) and potassium carbonate (10.0 g, 70.9 mmol). The resulting mixture was stirred for 16 h at 100° C. and then cooled to room temperature. The reaction mixture was poured into water (200 mL) and then extracted with ethyl acetate (3×150 mL). The combined organic layers were dried over anhydrous sodium sulfate, filtered, and concentrated under vacuum. The crude product was purified by silica gel chromatography (eluting with 0:100 to 20:80 ethyl acetate/petroleum ether) to afford 4-bromo-2-(2-bromo-1,1,2,2-tetrafluoroethoxy)-1-methoxybenzene as light a yellow oil (15.0 g, 81%). GCMS (EI, m/z): 380, 382 [M+H]⁺.

### Step 2. 4-bromo-2-(2-bromo-1,1,2,2-tetrafluoroethoxy)phenol

To a solution of 4-bromo-2-(2-bromo-1,1,2,2-tetrafluoroethoxy)-1-methoxybenzene (10 g, 25.1 mmol) in acetic acid (72 mL) was added hydrobromic acid (28 mL, 48% in acetic acid). The resulting mixture was stirred for 16 h at 120° C. and then cooled to room temperature. The reaction mixture was poured into water (200 mL) and then extracted with ethyl acetate (3×150 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford 8-fluoro-2,3-dihydro-1,4-benzodioxine-6-sulfonyl chloride as a yellow oil (5.0 g, 54%). GCMS (EI, m/z): 366, 368, 370 [M+H]⁺.

### Step 3. 6-bromo-2,2,3,3-tetrafluoro-2,3-dihydro-1,4-benzodioxine

To a solution of 4-bromo-2-(2-bromo-1,1,2,2-tetrafluoroethoxy)-1-methoxybenzene (4.0 g, 10.3 mmol) in MeOH (6 mL) was added potassium methoxide (1.08 g, 15.4 mmol). The resulting mixture was stirred for 0.5 h at room temperature and concentrated under vacuum. The residue was dissolved in tetrahydrothiophene 1,1-dioxide (20 mL). The resulting solution was stirred for 14 h at 140° C. and then cooled to room temperature. The reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 6-bromo-2,2,3,3-tetrafluoro-2,3-dihydro-1,4-benzodioxine as a light yellow oil (200 mg, 7%). GCMS (EI, m/z): 286, 288 [M+H]⁺.

### Step 4. 2,2,3,3-tetrafluoro-2,3-dihydro-1,4-benzodioxine-6-sulfonyl Chloride

To a solution of n-BuLi (0.63 mL, 2.5 M in heptane) was added n-Bu₂Mg (1.57 mL, 1.0 M in hexane). The resulting mixture was stirred for 10 min at room temperature before adding 6-bromo-2,2,3,3-tetrafluoro-2,3-dihydro-1,4-benzodioxine (300 mg, 0.94 mmol) in tetrahydrofuran (1 mL) dropwise with stirring at −10° C. The resulting mixture was warmed to room temperature and stirred for 2 h. The reaction was added to a cooled solution of sulfonylchloride (1.7 mL) in toluene (3 mL) dropwise with stirring at −10° C. The resulting mixture was stirred for 0.5 h and then poured into saturated aqueous ammonium chloride (15 mL). The product was extracted with ethyl acetate (3×10 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford 2,2,3,3-tetrafluoro-2,3-dihydro-1,4-benzodioxine-6-sulfonyl chloride as a light yellow oil (120 mg, 42%). GCMS (EI, m/z): 306 [M+H]⁺.

### Step 5. Tert-butyl 5-[(2,2,3,3-tetrafluoro-2,3-dihydro-1,4-benzodioxin-6-yl)sulfonyl]-1H,2H,3H,4H,5H,6H-pyrrolo[3,4-c]pyrrole-2-carboxylate

To a solution of 2,2,3,3-tetrafluoro-2,3-dihydro-1,4-benzodioxine-6-sulfonyl chloride (100 mg, 0.29 mmol) in dichloromethane (2 mL) was added DIEA (0.11 mL, 0.65 mmol) and 2-tert-butyl-1H,3H,4H,5H,6H,7H,8H-2lambda3,4,7-pyrrolo[3,4-e][1lambda3,3]oxazepin-3-one (69 mg, 0.31 mmol) in DCM (5 mL) added dropwise with stirring. The resulting mixture was stirred for 14 h at room temperature. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford tert-butyl 5-[(2,2,3,3-tetrafluoro-2,3-dihydro-1,4-benzodioxin-6-yl)sulfonyl]-1H,2H,3H,4H,5H,6H-pyrrolo[3,4-c]pyrrole-2-carboxylate as a yellow solid (60 mg, 42%). LCMS (ES, m/z): 481 [M+H]⁺.

### Step 6. 2-[(2,2,3,3-tetrafluoro-2,3-dihydro-1,4-benzodioxin-6-yl)sulfonyl]-1H,2H,3H,4H,5H,6H-pyrrolo[3,4-c]pyrrole TFA Salt

To a solution of tert-butyl 5-[(2,2,3,3-tetrafluoro-2,3-dihydro-1,4-benzodioxin-6-yl)sulfonyl]-1H,2H,3H,4H,5H,6H-pyrrolo[3,4-c]pyrrole-2-carboxylate (40 mg, 0.08 mmol) in dichloromethane (5 mL) was added trifluoroacetic acid (1 mL). The resulting solution was stirred for 1 h at room temperature and concentrated under vacuum to afford 2-[(2,2,3,3-tetrafluoro-2,3-dihydro-1,4-benzodioxin-6-yl)sulfonyl]-1H,2H,3H,4H,5H,6H-pyrrolo[3,4-c]pyrrole TFA salt as yellow oil (50 mg, crude). LCMS (ES, m/z): 381 [M+H]⁺.

### Intermediate 58-1. 5-(4-fluorophenyl)-1-methyl-1H-pyrazole-3-carbaldehyde

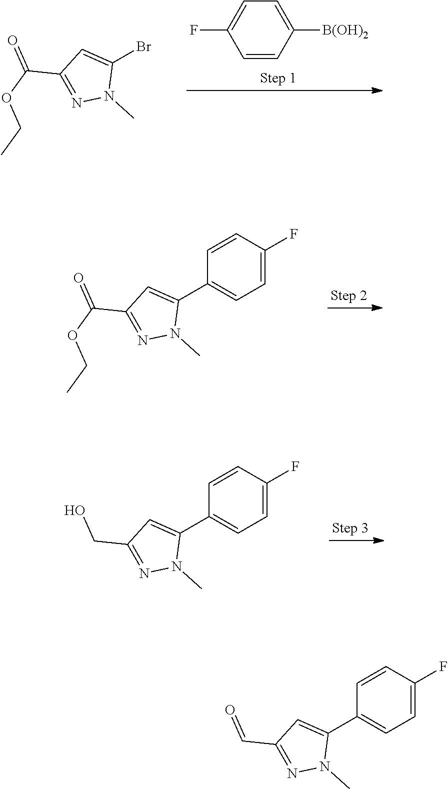 [SMILES: CCOC(=O)c1cc(Br)n(C)n1 ; OB(O)c1ccc(F)cc1 ; CCOC(=O)c1cc(-c2ccc(F)cc2)n(C)n1 ; Cn1nc(CO)cc1-c1ccc(F)cc1 ; Cn1nc(C=O)cc1-c1ccc(F)cc1]

### Step 1. Ethyl 5-(4-fluorophenyl)-1-methyl-1H-pyrazole-3-carboxylate

To a solution of ethyl 5-bromo-1-methyl-1H-pyrazole-3-carboxylate (1.00 g, 4.20 mmol) in 1,4-dioxane (12 mL) was added (4-fluorophenyl)boronic acid (724 mg, 5.07 mmol), potassium carbonate (1.19 g, 8.61 mmol), Pd(dppf)Cl₂.CH₂Cl₂ (352 mg, 0.43 mmol) and water (4 mL). The mixture was stirred for 3 h at 100° C., cooled to room temperature and then poured into water (5 mL). The product was extracted with ethyl acetate (3×15 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford ethyl 5-(4-fluorophenyl)-1-methyl-1H-pyrazole-3-carboxylate as a yellow solid (900 mg, 86%). LCMS (ES, m/z): 249 [M+H]⁺.

### Step 2. (5-(4-fluorophenyl)-1-methyl-1H-pyrazol-3-yl)methanol

To a solution of ethyl 5-(4-fluorophenyl)-1-methyl-1H-pyrazole-3-carboxylate (900 mg, 3.55 mmol) in THF (5 mL) was added lithium aluminium hydride (152 mg, 4.00 mmol) at 0° C. The resulting mixture was stirred for 5 min at 0° C. The reaction was then quenched by careful addition of Na₂SO₄.10H₂O (500 mg). The resulting mixture was filtered and concentrated under vacuum to afford (5-(4-fluorophenyl)-1-methyl-1H-pyrazol-3-yl)methanol as a yellow solid (700 mg, 96%). LCMS (ES, m/z): 207 [M+H]⁺.

### Step 3. 5-(4-fluorophenyl)-1-methyl-1H-pyrazole-3-carbaldehyde

To a solution of (5-(4-fluorophenyl)-1-methyl-1H-pyrazol-3-yl)methanol (650 mg, 2.99 mmol) in dichloromethane (15 mL) was added DMP (2.67 g, 6.23 mmol) at 0° C. The resulting mixture was stirred for 3 h at 25° C. The reaction mixture was poured into water (5 mL) and then extracted with ethyl acetate (3×15 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford ethyl 5-(4-fluorophenyl)-1-methyl-1H-pyrazole-3-carbaldehyde as an off-white solid (600 mg, 98%). LCMS (ES, m/z): 205 [M+H]⁺.

The Intermediates in Table 15 were synthesized according to the procedure described for Intermediate 58-1 above.

TABLE 15

| | | LCMS: (ESI) |
|---|---|---|
| Intermediate | Structure and Name | m/z [M + H]⁺ |
| 58-2 | 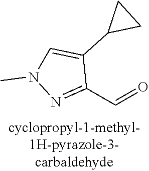 | 151 |

### Intermediate 59-1. 2,3-dihydro-1-benzofuran-4-carbaldehyde

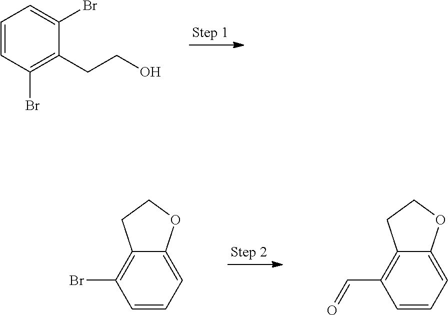 [SMILES: OCCc1c(Br)cccc1Br ; Brc1cccc2c1CCO2 ; O=Cc1cccc2c1CCO2]

### Step 1. 4-bromo-2,3-dihydro-1-benzofuran

To a solution of 2-(2,6-dibromophenyl)ethan-1-ol (600 mg, 2.14 mmol) in 1,4-dioxane (10 mL) was added cuprous iodide (41 mg, 0.22 mmol), and lithium t-butoxide (520 mg, 6.50 mmol). The mixture was stirred for 2 days at 110° C. and then cooled to room temperature. The reaction mixture was filtered and concentrated under vacuum. The resulting crude product was purified by silica gel chromatography (eluting with 0:100 to 5:95 ethyl acetate/petroleum ether) to afford 4-bromo-2,3-dihydro-1-benzofuran as a yellow oil (0.3 g, 45%). GCMS (EI, m/z): 198, 200 [M]⁺.

### Step 2. 2,3-dihydro-1-benzofuran-4-carbaldehyde

To a solution of 4-bromo-2,3-dihydro-1-benzofuran (800 mg, 2.57 mmol) in THF (20 mL) was added n-BuLi (2 mL, 2.5 M in n-hexane) dropwise with stirring at −78° C. After stirring for 10 min at −78° C., DMF (0.40 mL, 5.20 mmol) was added and stirred for 1 h. The reaction mixture was poured into saturated aqueous ammonium chloride (10 mL) and then extracted with ethyl acetate (3×20 mL). The combined organic layers were dried over anhydrous sodium sulfate, filtered, and concentrated under vacuum. The resulting crude product was purified by prep-TLC (eluting with 1:5 ethyl acetate/petroleum ether) to afford 2,3-dihydro-1-benzofuran-4-carbaldehyde as a yellow oil (410 mg, 97%). LCMS (ES, m/z): 149 [M+H]⁺.

### Intermediate 60-1. 2-acetamido-2-(2-methyl-1,3-benzoxazol-4-yl)acetic Acid

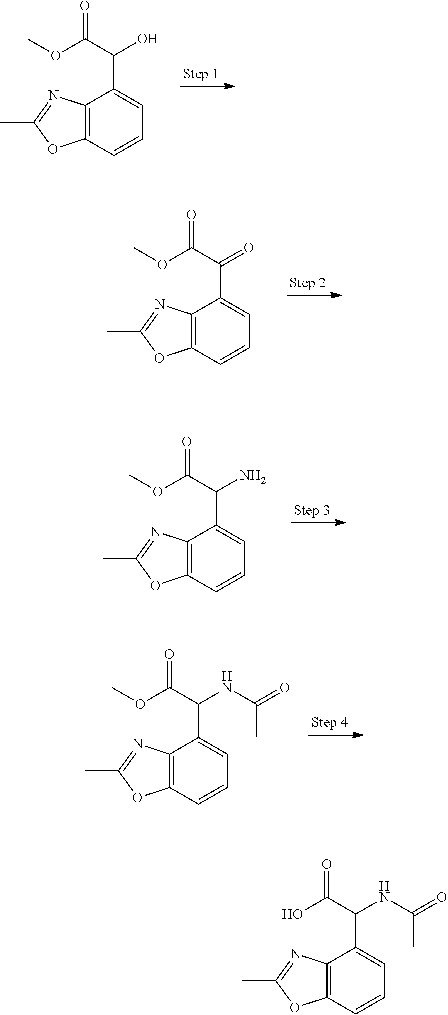 [SMILES: COC(=O)C(O)c1cccc2oc(C)nc12 ; COC(=O)C(=O)c1cccc2oc(C)nc12 ; COC(=O)C(N)c1cccc2oc(C)nc12 ; COC(=O)C(NC(C)=O)c1cccc2oc(C)nc12 ; CC(=O)NC(C(=O)O)c1cccc2oc(C)nc12]

### Step 1. Methyl 2-(2-methyl-1,3-benzoxazol-4-yl)-2-oxoacetate

To a stirred solution of methyl 2-hydroxy-2-(2-methyl-1,3-benzoxazol-4-yl)acetate (600 mg, 2.71 mmol) in dichloromethane (15 mL) was added DMP (1.27 g, 2.99 mmol). After 16 h, the reaction mixture was poured into brine (5 mL) and then extracted with ethyl acetate (3×20 mL). The combined organic layers were dried over anhydrous sodium sulfate, filtered, and concentrated under vacuum to afford methyl 2-(2-methyl-1,3-benzoxazol-4-yl)-2-oxoacetate as a light yellow oil (400 mg, 73%). LCMS (ES, m/z): 220 [M+H]⁺.

### Step 2. Methyl 2-amino-2-(2-methyl-1,3-benzoxazol-4-yl)acetate

To a solution of methyl 2-(2-methyl-1,3-benzoxazol-4-yl)-2-oxoacetate (300 mg, 1.37 mmol) in acetic acid (6 mL) was added phenylhydrazine (178 mg, 1.65 mmol), and zinc powder (891 mg, 13.6 mmol). The resulting mixture was stirred for 2 d. The reaction was filtered and concentrated under vacuum. The resulting crude product was purified by silica gel chromatography (eluting with 0:100 to 4:96 MeOH/DCM) to afford methyl 2-amino-2-(2-methyl-1,3-benzoxazol-4-yl)acetate as a brown oil (200 mg, 66%). LCMS (ES, m/z): 221 [M+H]⁺.

### Step 3. Methyl 2-acetamido-2-(2-methyl-1,3-benzoxazol-4-yl)acetate

To a solution of methyl 2-amino-2-(2-methyl-1,3-benzoxazol-4-yl)acetate (100 mg, 0.45 mmol) in dichloromethane (2 mL) was added acetic anhydride (0.05 mL, 0.55 mmol), and TEA (0.19 mL, 1.36 mmol). The resulting mixture was stirred for 1 h at room temperature and concentrated under vacuum. The resulting crude product was purified by silica gel chromatography (eluting with 0:100 to 10:90 ethyl acetate/petroleum ether) to afford methyl 2-acetamido-2-(2-methyl-1,3-benzoxazol-4-yl)acetate as a light yellow oil (90 mg, 76%). LCMS (ES, m/z): 263 [M+H]⁺.

### Step 4. 2-acetamido-2-(2-methyl-1,3-benzoxazol-4-yl)acetic Acid

To a solution of methyl 2-acetamido-2-(2-methyl-1,3-benzoxazol-4-yl)acetate (90 mg, 0.34 mmol) in tetrahydrofuran (0.75 mL) and water (0.75 mL) was added lithium hydroxide (83 mg, 3.47 mmol). The resulting mixture was stirred for 16 h. The reaction mixture was washed with diethyl ether (1×8 mL) and then acidified to pH=5 with saturated citric acid. The resulting solution was extracted with ethyl acetate (2×8 mL). The organic layers were combined, dried over anhydrous sodium sulfate, filtered, and concentrated under vacuum to afford 2-acetamido-2-(2-methyl-1,3-benzoxazol-4-yl)acetic acid as a light yellow oil (70 mg, 82%). LCMS (ES, m/z): 249 [M+H]⁺.

### Intermediate 61-1. 3-methyl-1,2-benzoxazole-4-carbaldehyde

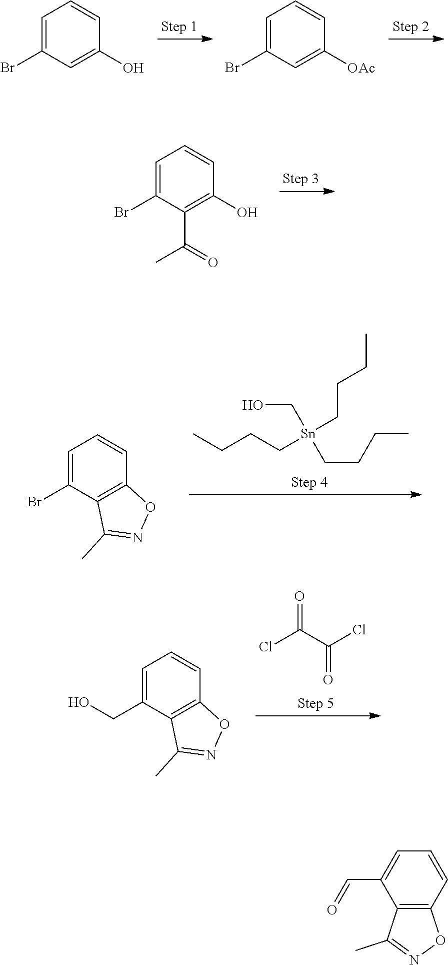

### Step 1. 3-bromophenyl Acetate

To a solution of 3-bromophenol (10 g, 57.2 mmol) in acetic anhydride (17.4 mL, 184 mmol) was added sulfuric acid (0.2 mL). The resulting mixture was stirred for 18 h. The reaction mixture was poured into hydrochloric acid (100 mL, 2 wt %) and then extracted with ethyl acetate (3×100 mL). The combined organic layers were washed with aqueous sodium bicarbonate (2 N) twice and brine, dried over anhydrous sodium sulfate, filtered and concentrated under vacuum to afford 3-bromophenyl acetate as a colorless oil (13 g, crude). LCMS (ES, m/z): 215, 217 [M+H]⁺.

### Step 2. 1-(2-bromo-6-hydroxyphenyl)ethan-1-one

To 3-bromophenyl acetate (13 g, 56.2 mmol) was added trichloroalumane (12 g, 90.0 mmol). The resulting mixture was stirred for 2 h at 145° C. After cooling to room temperature, the reaction mixture was poured into hydrochloric acid (32 mL, 5 wt %) and extracted with DCM (3×50 mL). The combined organic layers were washed with sodium hydroxide (100 mL, 5 N). The aqueous layer was acidified to pH=2 with concentrated hydrochloric acid and then extracted with ethyl acetate (5×50 mL). The combined organic layers were dried over anhydrous sodium sulfate, filtered and concentrated under vacuum to afford 1-(2-bromo-6-hydroxyphenyl)ethan-1-one as a white solid (7.4 g, 61%). LCMS (ES, m/z): 215, 217 [M+H]⁺.

### Step 3. 4-bromo-3-methyl-1,2-benzoxazole

To 1-(2-bromo-6-hydroxyphenyl)ethan-1-one (3 g, 13.0 mmol) was added ammonia solution (15 mL, 7 M in methanol). The resulting mixture was stirred for 3 h at 25° C. The generated solids were collected by filtration, washed with diethyl ether and then dissolved with THF (20 mL). Then N-Chlorosuccinimide (2.1 g, 15.7 mmol) and potassium carbonate (2.85 g, 20.5 mmol) was added. The resulting mixture was stirred for 18 h at 25° C. The reaction mixture was poured into water (50 mL) and then extracted with ethyl acetate (3×50 mL). The combined organic layers were dried over anhydrous sodium sulfate, filtered and concentrated under vacuum. The resulting crude product was purified by silica gel chromatography (eluting with 0:100 to 20/80 ethyl acetate/petroleum ether) to afford 4-bromo-3-methyl-1,2-benzoxazole as a yellow solid (1.8 g, 65%). LCMS (ES, m/z): 212, 214 [M+H]⁺.

### Step 4. (3-methyl-1,2-benzoxazol-4-yl)methanol

To a solution of 4-bromo-3-methyl-1,2-benzoxazole (1.72 g, 8.11 mmol) in 1,2-dioxane (20 mL) was added Pd(PPh₃)₄(938 mg, 0.81 mmol), and (tributylstannyl)methanol (3.39 g, 10.6 mmol). The resulting mixture was stirred for 16 h at 100° C. After cooling to room temperature, the reaction mixture was poured into saturated aqueous ammonium chloride (10 mL) and then extracted with ethyl acetate (3×10 mL). The combined organic layers were dried over anhydrous sodium sulfate, filtered and concentrated under vacuum. The resulting crude product was purified by silica gel chromatography (eluting with 0:100 to 20/80 ethyl acetate/petroleum ether) to afford (3-methyl-1,2-benzoxazol-4-yl)methanol as a light yellow oil (900 mg, 68%). LCMS (ES, m/z): 164 [M+H]⁺.

### Step 5. 3-methyl-1,2-benzoxazole-4-carbaldehyde

To a solution of oxalyl chloride (1.40 g, 11.1 mmol) in dichloromethane (10 mL) was added DMSO (1.17 mL, 16.5 mmol) dropwise with stirring at −78° C. The reaction was treated with 1,2-benzoxazol-4-ylmethanol (900 mg, 6.03 mmol) in DCM (5 mL). After 2 h, TEA (4.60 mL, 33.1 mmol) was added. The resulting mixture was warmed to room temperature for 2 h. The reaction mixture was poured into brine (5 mL) and then extracted with dichloromethane (3×10 mL). The combined organic layers were dried over anhydrous sodium sulfate, filtered and concentrated under vacuum. The resulting crude product was purified by silica gel chromatography (eluting with 1:5 ethyl acetate/petroleum ether) to afford 3-methyl-1,2-benzoxazole-4-carbaldehyde as a colorless oil (370 mg, 38%). LCMS (ES, m/z): 162 [M+H]⁺.

### Intermediate 63-1. tert-butyl (R)-(3-(5-((2,3-dihydrobenzo[b][1,4]dioxin-6-yl)sulfonyl)-3,4,5,6-tetrahydropyrrolo[3,4-c]pyrrol-2(1H)-yl)-3-oxo-2-phenylpropyl)carbamate

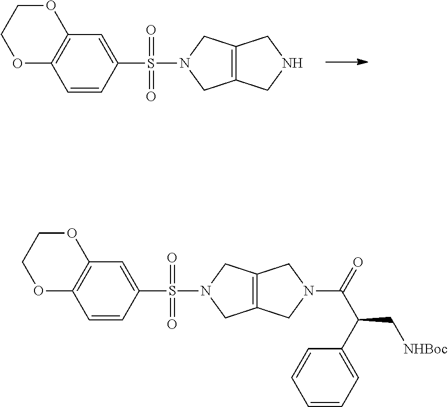 [SMILES: O=S(=O)(c1ccc2c(c1)OCCO2)N1CC2=C(CNC2)C1 ; CC(C)(C)OC(=O)NC[C@H](C(=O)N1CC2=C(C1)CN(S(=O)(=O)c1ccc3c(c1)OCCO3)C2)c1ccccc1]

In a dry 25 ml RBF under N₂ was added 2-((2,3-dihydrobenzo[b][1,4]dioxin-6-yl)sulfonyl)-1,2,3,4,5,6-hexahydropyrrolo[3,4-c]pyrrole (200 mg, 0.649 mmol), (R)-3-((tert-butoxycarbonyl)amino)-2-phenylpropanoic acid (189 mg, 0.713 mmol), DMF (1 mL), DIEA (170 μl, 0.973 mmol) and HATU (271 mg, 0.713 mmol). After 3 h, the reaction was diluted with 50 ml of saturated aqueous bicarbonate solution (50 mL) and extracted with EtOAc (4×20 mL). The extracts were dried over Na₂SO₄, filtered and the solvent was removed in vacuo to afford 524 mg of a brown gummy solid. The crude material was purified by a Biotage SNAP-25 Silica column and eluted with an EtOAc/Hexane gradient (0-100% EtOAc). The desired product was isolated affording 309 mg of a white solid.

The Intermediates in Table 16 were synthesized according to the procedure described for Intermediate 63-1 above.

TABLE 16

| | | LCMS |
|---|---|---|
| | | (ESI) m/z |
| Intermediate | Structure and Name | [M + H]⁺ |
| 63-2 | 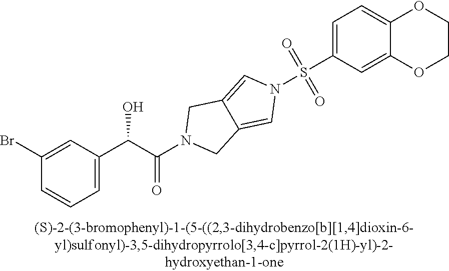 [SMILES: O=C([C@@H](O)c1cccc(Br)c1)N1Cc2cn(S(=O)(=O)c3ccc4c(c3)OCCO4)cc2C1] | 519 |
| 63-3 | 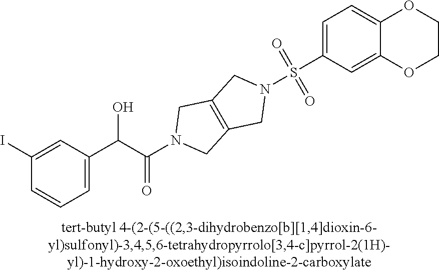 [SMILES: O=C(C(O)c1cccc(I)c1)N1CC2=C(C1)CN(S(=O)(=O)c1ccc3c(c1)OCCO3)C2] | 569 |
| 63-4 | 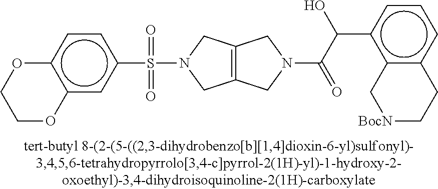 | 598 |
| 63-5 | 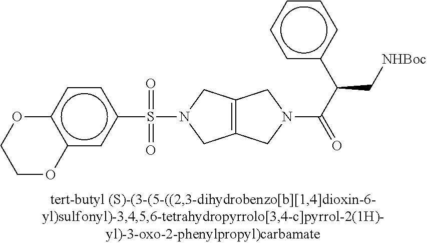 | 556 |
| 63-6 | 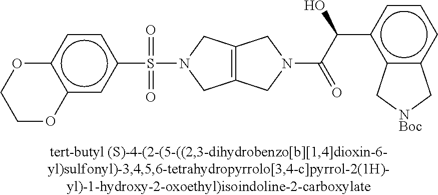 | 584 |
| 63-7 | 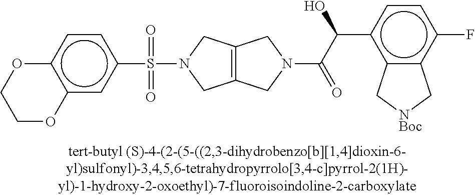 | 602 |
| 63-8 | 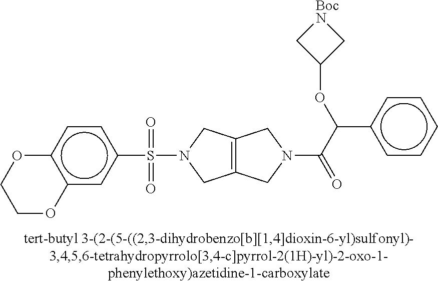 | 598 |
| 63-9 | 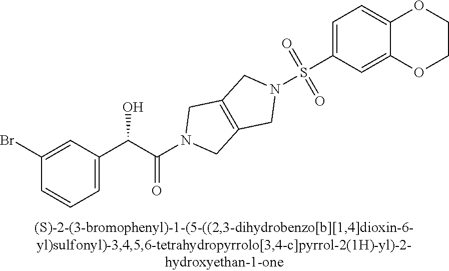 [SMILES: O=C([C@@H](O)c1cccc(Br)c1)N1CC2=C(C1)CN(S(=O)(=O)c1ccc3c(c1)OCCO3)C2] | 521 |
| 63-10 | 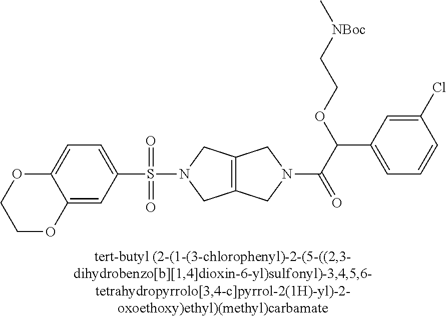 [SMILES: CN(CCOC(C(=O)N1CC2=C(C1)CN(S(=O)(=O)c1ccc3c(c1)OCCO3)C2)c1cccc(Cl)c1)C(=O)OC(C)(C)C] | 634 |

### Intermediate 66. tert-butyl 5-[3-[(1S)-2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]phenyl]-octahydropyrrolo[3,4-c]pyrrole-2-carboxylate

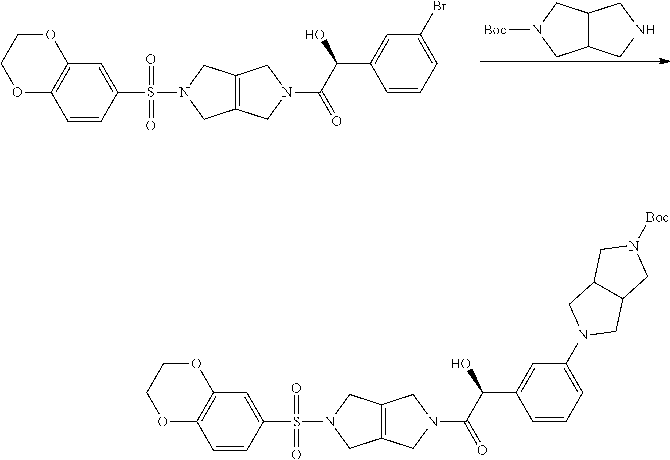 [SMILES: O=C([C@@H](O)c1cccc(Br)c1)N1CC2=C(C1)CN(S(=O)(=O)c1ccc3c(c1)OCCO3)C2 ; CC(C)(C)OC(=O)N1CC2CNCC2C1 ; CC(C)(C)OC(=O)N1CC2CN(c3cccc([C@H](O)C(=O)N4CC5=C(C4)CN(S(=O)(=O)c4ccc6c(c4)OCCO6)C5)c3)CC2C1]

To a solution of (2S)-2-(3-bromophenyl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (100 mg, 0.20 mmol) in toluene (5 mL) was added tert-butyl octahydropyrrolo[3,4-c]pyrrole-2-carboxylate (609 mg, 2.87 mmol), RuPhos 2G (15 mg, 0.02 mmol), RuPhos (18 mg, 0.04 mmol), and cesium carbonate (189 mg, 0.58 mmol). The resulting mixture was stirred overnight at 100° C. After cooling to room temperature, the reaction was concentrated under vacuum. The crude product was purified by silica gel chromatography (eluting with 1:25 MeOH/DCM) to afford tert-butyl 5-[3-[(1S)-2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]phenyl]-octahydropyrrolo-[3,4-c]pyrrole-2-carboxylate as a light yellow solid (100 mg, 80%). LCMS (ES, m/z): 653 [M+H]⁺.

The Intermediates in Table 18 were synthesized according to the procedure described for Intermediate 66 above.

TABLE 18

| | | LCMS: |
|---|---|---|
| | | (ESI) m/z |
| Intermediate | Structure and Name | [M + H]⁺ |
| 66-2 | 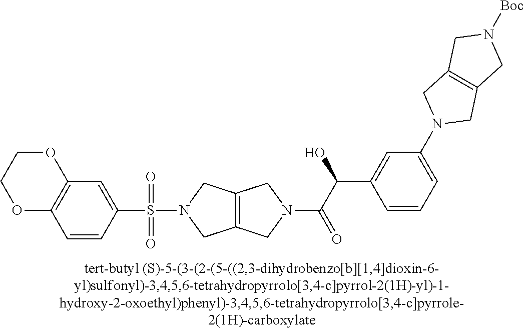 [SMILES: CC(C)(C)OC(=O)N1CC2=C(C1)CN(c1cccc([C@H](O)C(=O)N3CC4=C(C3)CN(S(=O)(=O)c3ccc5c(c3)OCCO5)C4)c1)C2] | 651 |
| 66-3 | 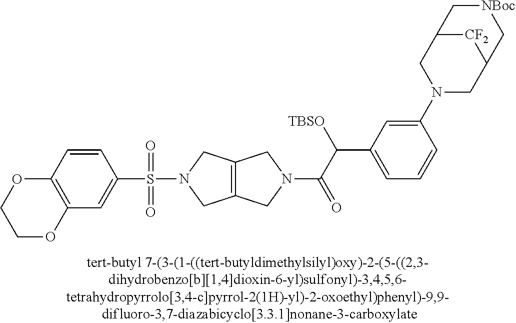 [SMILES: CC(C)(C)OC(=O)N1CC2CN(c3cccc(C(O[Si](C)(C)C(C)(C)C)C(=O)N4CC5=C(C4)CN(S(=O)(=O)c4ccc6c(c4)OCCO6)C5)c3)CC(C1)C2(F)F] | 817 |
| 66-4 | 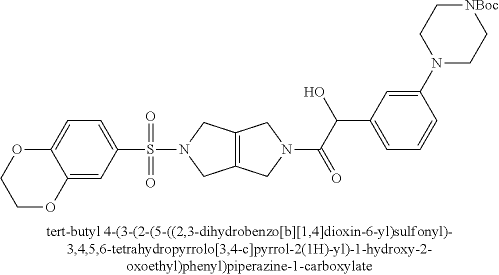 [SMILES: CC(C)(C)OC(=O)N1CCN(c2cccc(C(O)C(=O)N3CC4=C(C3)CN(S(=O)(=O)c3ccc5c(c3)OCCO5)C4)c2)CC1] | 627 |

### Intermediate 67. 2-acetamido-2-(benzo[d]isoxazol-3-yl)acetic Acid

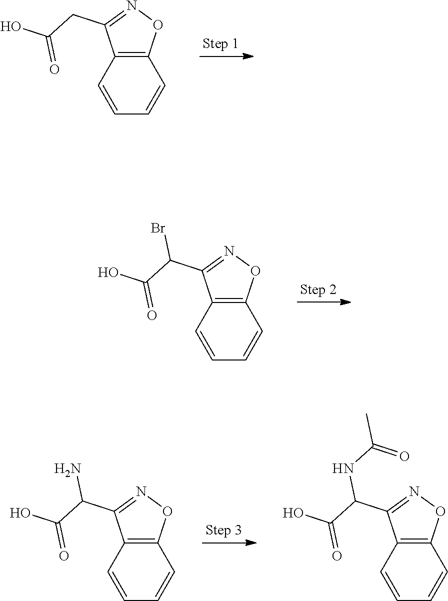 [SMILES: O=C(O)Cc1noc2ccccc12 ; O=C(O)C(Br)c1noc2ccccc12 ; NC(C(=O)O)c1noc2ccccc12 ; CC(=O)NC(C(=O)O)c1noc2ccccc12]

### Step 1. 2-(1,2-benzoxazol-3-yl)-2-bromoacetic Acid

To a solution of 2-(1,2-benzoxazol-3-yl)acetic acid (3 g, 16.9 mmol) in acetic acid (30 mL), was added bromine (3.21 g, 20.1 mmol) at 0-10° C. The solution was stirred for 16 h at room temperature and then concentrated under vacuum. The crude product was washed with DCM and dried under vacuum to afford 2-(1,2-benzoxazol-3-yl)-2-bromoacetic acid as an off-white solid (3.2 g, 74%). LCMS (ES, m/z): 509, 511, 513 [2M−H]⁻.

### Step 2. 2-amino-2-(1,2-benzoxazol-3-yl)acetic Acid

To a solution of 2-(1,2-benzoxazol-3-yl)-2-bromoacetic acid (500 mg, 1.95 mmol) in methanol (5 mL) was added ammonia (15 mL, 7 M in MeOH). The resulting solution was stirred for 16 h at 40° C. and then concentrated under vacuum. The crude product was washed with MeOH and dried under vacuum to afford 2-amino-2-(1,2-benzoxazol-3-yl)acetic acid as an off-white solid (0.7 g, 47%). LCMS (ES, m/z): 193 [M+H]⁺.

### Step 3. 2-acetamido-2-(benzo[d]isoxazol-3-yl)acetic Acid

To a solution of 2-amino-2-(1,2-benzoxazol-3-yl) acetic acid (600 mg, 3.12 mmol) in MeOH (25 mL) and water (5 mL), was added sodium hydroxide (500 mg, 12.5 mmol) and acetic anhydride (1.59 g, 15.6 mmol) at 0° C. The mixture was stirred for 30 min at 0° C. and concentrated under vacuum. The crude product was purified by reversed phase chromatography (eluting with 0:100 to 50:50 MeCN/water (containing 10 mM NH₄HCO₃)) to afford 2-(1,2-benzoxazol-3-yl)-2-acetamido acetic acid as a white solid (0.6 g, 82%). LCMS (ES, m/z): 235 [M+H]⁺.

### Intermediate 68. 4-[[(tert-butoxy)carbonyl]amino]-2-(3-chlorophenyl)butanoic Acid

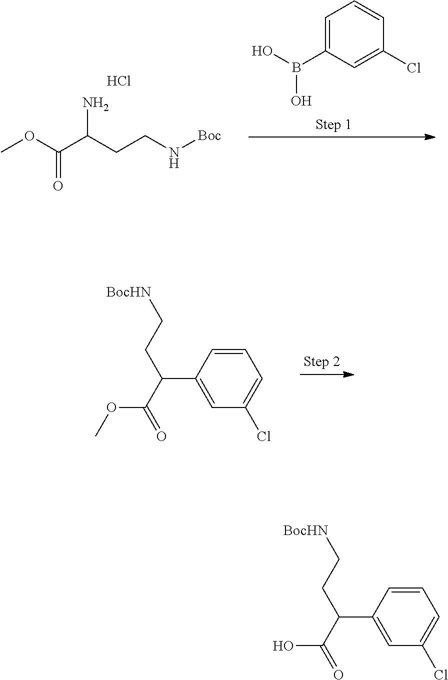 [SMILES: Cl ; OB(O)c1cccc(Cl)c1 ; COC(=O)C(N)CCNC(=O)OC(C)(C)C ; COC(=O)C(CCNC(=O)OC(C)(C)C)c1cccc(Cl)c1 ; CC(C)(C)OC(=O)NCCC(C(=O)O)c1cccc(Cl)c1]

### Step 1. Methyl 4-[[(tert-butoxy)carbonyl]amino]-2-(3-chlorophenyl)butanoate

To a solution of methyl 2-amino-4-[[(tert-butoxy)carbonyl]amino]butanoate hydrochloride (2.00 g, 7.07 mmol) in THF (2 mL) was added water (1 mL), sodium nitrite (858 mg, 12.4 mmol), (3-chlorophenyl)boronic acid (776 mg, 4.96 mmol), and potassium formate (417 mg, 4.96 mmol). The resulting mixture was stirred for 48 h at 80° C. After cooling to room temperature, the reaction mixture was poured into saturated aqueous ammonium chloride solution (10 mL) and then extracted with ethyl acetate (3×10 mL). The combined organic layers were dried over anhydrous sodium sulfate, filtered, and concentrated under vacuum. The crude product was purified by prep-TLC (eluting with 1:1 ethyl acetate/petroleum ether) to afford methyl 4-[[(tert-butoxy)carbonyl]amino]-2-(3-chlorophenyl)butanoate as yellow oil (827 mg, 36%). LCMS (ES, m/z): 328, 330 [M+H]⁺.

### Step 2. 4-[[(tert-butoxy)carbonyl]amino]-2-(3-chlorophenyl)butanoic Acid

To a solution of ethyl methyl 4-[[(tert-butoxy)carbonyl]amino]-2-(3-chlorophenyl)butanoate (827 mg, 2.27 mmol) in THF (3 mL) and water (3 mL) was added lithium hydroxide (303 mg, 12.7 mmol). The resulting mixture was stirred for 18 h at room temperature. The reaction mixture was washed with diethyl ether (1×5 mL) and then acidified to pH=7 with saturated aqueous citric acid. The resulting solution was extracted with ethyl acetate (3×10 mL). The combined organic layers were dried over anhydrous sodium sulfate, filtered, and concentrated under vacuum to afford 4-[[(tert-butoxy)carbonyl]amino]-2-(3-chlorophenyl)butanoic acid as a yellow oil (524 mg, 75%). LCMS (ES, m/z): 314, 316 [M+H]⁺.

### Intermediate 70. 2-[(8-fluoro-2,3-dihydro-1,4-benzodioxin-6-yl)sulfonyl]-1H,2H,3H,4H,5H,6H-pyrrolo[3,4-c]pyrrole

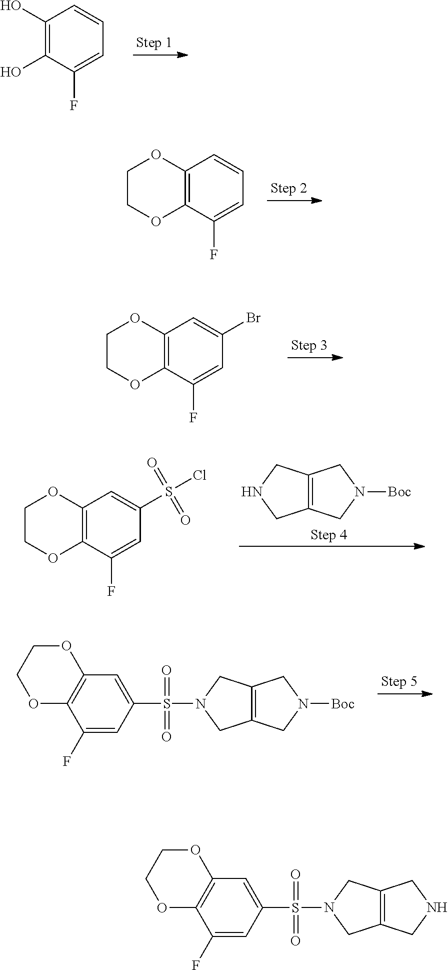

### Step 1. 5-fluoro-2,3-dihydro-1,4-benzodioxine

To a solution of 3-fluorobenzene-1,2-diol (4.40 g, 34.4 mmol) in N,N-dimethylformamide (50 mL) was added potassium carbonate (14.3 g, 103 mmol) and 1,2-dibromoethane (14.8 mL, 172 mmol). The resulting mixture was stirred for 2 h at 80° C. and then cooled to room temperature. The reaction mixture was poured into water (50 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 5-fluoro-2,3-dihydro-1,4-benzodioxine as a yellow oil (4.30 g, 81%). LCMS (ES, m/z): 155 [M+H]⁺.

### Step 2. 7-bromo-5-fluoro-2,3-dihydro-1,4-benzodioxine

To a solution of 5-fluoro-2,3-dihydro-1,4-benzodioxine (4.40 g, 28.6 mmol) in methanol (40 mL) was added NBS (5.09 g, 28.6 mmol). The resulting mixture was stirred for 1 h at 65° C. After cooling to room temperature, the reaction mixture was concentrated under vacuum. The reaction was poured into water (20 mL) and extracted with ethyl acetate (3×30 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 7-bromo-5-fluoro-2,3-dihydro-1,4-benzodioxine as a light yellow solid (5.00 g, 75%). LCMS (ES, m/z): 233, 235 [M+H]⁺.

### Step 3. 8-fluoro-2,3-dihydro-1,4-benzodioxine-6-sulfonyl Chloride

To a solution of 7-bromo-5-fluoro-2,3-dihydro-1,4-benzodioxine (2.00 g, 8.58 mmol) in tetrahydrofuran (20 mL), was added n-BuLi (3.62 mL, 2.5 M in hexane) dropwise at −78° C. After stirring for 30 min at −78° C., SO₂ (g) was bubbled through the solution in for 30 min. The mixture was warmed to 20° C., stirred for 15 min and concentrated under vacuum. The residue was dissolved in dichloromethane (20 mL) and treated with NCS (1.26 g, 9.44 mmol). After 45 min at 0 degrees, the reaction was poured into water (20 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 100:0 ethyl acetate/petroleum ether) to afford 8-fluoro-2,3-dihydro-1,4-benzodioxine-6-sulfonyl chloride as a yellow solid (1.20 g, 55%). LCMS (ES, m/z): 253, 255 [M+H]⁺.

### Step 4. Tert-butyl 5-[(8-fluoro-2,3-dihydro-1,4-benzodioxin-6-yl)sulfonyl]-1H,2H,3H,4H,5H,6H-pyrrolo [3,4-c]pyrrole-2-carboxylate

To a solution of tert-butyl 1H,2H,3H,4H,5H,6H-pyrrolo[3,4-c]pyrrole-2-carboxylate (138 mg, 0.66 mmol) in dichloromethane (3 mL), was added triethylamine (0.27 mL, 1.98 mmol) and 8-fluoro-2,3-dihydro-1,4-benzodioxine-6-sulfonyl chloride (200 mg, 0.79 mmol). The solution was stirred for 1 h at room temperature and concentrated under vacuum. The crude product was purified by silica gel chromatography (eluting with 0:100 to 100:0 ethyl acetate/petroleum ether) to afford tert-butyl 5-(8-fluoro-2,3-dihydro-1,4-benzodioxine-6-sulfonyl)-1H,2H,3H,4H,5H,6H-pyrrolo[3,4-c]pyrrole-2-carboxylate as a yellow solid (340 mg, 77%). LCMS (ES, m/z): 427 [M+H]⁺.

### Step 5. 2-[(8-fluoro-2,3-dihydro-1,4-benzodioxin-6-yl)sulfonyl]-1H,2H,3H,4H,5H,6H-pyrrolo[3,4-c]pyrrole

To a solution of tert-butyl 5-(8-fluoro-2,3-dihydro-1,4-benzodioxine-6-sulfonyl)-1H,2H,3H,4H,5H,6H-pyrrolo[3,4-c]pyrrole-2-carboxylate (340 mg, 0.61 mmol) in dichloromethane (5 mL) was added trifluoroacetic acid (1 mL). The resulting solution was stirred for 1 h at room temperature. The resulting mixture was concentrated under vacuum to afford 2-(8-fluoro-2,3-dihydro-1,4-benzodioxine-6-sulfonyl)-1H,2H,3H,4H,5H,6H-pyrrolo[3,4-c]pyrrole as a brown oil (60 mg, 30%). LCMS (ES, m/z): 327 [M+H]⁺.

### Intermediate 71. Tert-butyl 6-formyl-2,3-dihydrobenzo[b][1,4]oxazine-4-carboxylate

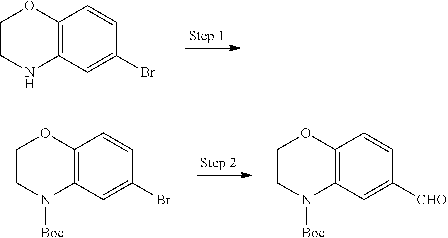 [SMILES: Brc1ccc2c(c1)NCCO2 ; CC(C)(C)OC(=O)N1CCOc2ccc(Br)cc21 ; CC(C)(C)OC(=O)N1CCOc2ccc(C=O)cc21]

### Step 1. Tert-butyl 6-bromo-2,3-dihydrobenzo[b][1,4]oxazine-4-carboxylate

To a solution of 6-bromo-3,4-dihydro-2H-1,4-benzoxazine (1.40 g, 6.54 mmol) in dichloromethane (10 mL) was added di-tert-butyl dicarbonate (1.71 g, 7.84 mmol), 4-dimethylaminopyridine (80 mg, 0.65 mmol) and TEA (1.81 mL, 13.0 mmol). The resulting solution was stirred overnight at room temperature and concentrated under vacuum. The resulting crude product was purified by prep-TLC (eluting with 1:5 ethyl acetate/petroleum ether) to afford tert-butyl 6-bromo-2,3-dihydrobenzo[b][1,4]oxazine-4-carboxylate as a white solid (1.40 g, 68%). LCMS (ES, m/z): 314, 316 [M+H]⁺.

### Step 2. Tert-butyl 6-formyl-2,3-dihydrobenzo[b][1,4]oxazine-4-carboxylate

To a solution of tert-butyl 6-bromo-2,3-dihydrobenzo[b][1,4]oxazine-4-carboxylate (3.13 g, 9.96 mmol) in tetrahydrofuran (40 mL) was added n-butyllithium (12 mL, 2.50 M in hexane) dropwise with stirring at −78° C. The resulting solution was stirred for 30 min and treated with N,N-dimethylformamide (23.8 mL, 308 mmol). The resulting solution was warmed to room temperature and stirred for 1 h before pouring the contents into water (50 mL). The product was extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 6-formyl-2,3-dihydrobenzo[b][1,4]oxazine-4-carboxylate as a light yellow oil (1.80 g, 69%). LCMS (ES, m/z): 264 [M+H]⁺.

### Intermediate 72. tert-butyl 2-(2,3-dihydro-1,4-benzodioxine-6-sulfonyl)-2H,4H,5H,6H-pyrrolo[3,4-c]pyrazole-5-carboxylate

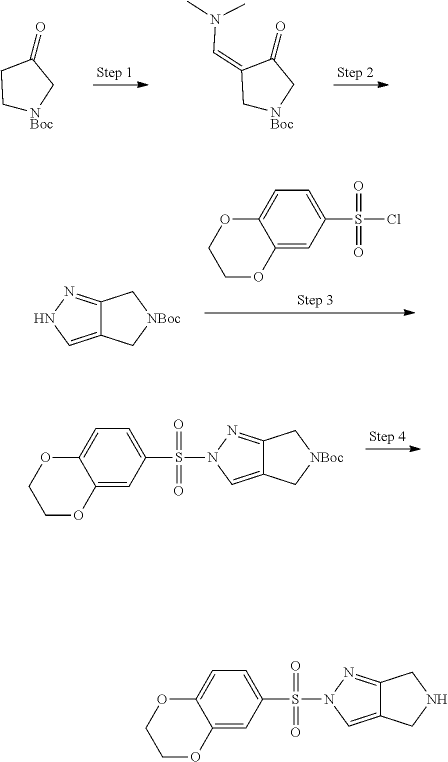 [SMILES: CC(C)(C)OC(=O)N1CCC(=O)C1 ; CN(C)/C=C1/CN(C(=O)OC(C)(C)C)CC1=O ; O=S(=O)(Cl)c1ccc2c(c1)OCCO2 ; CC(C)(C)OC(=O)N1Cc2c[nH]nc2C1 ; CC(C)(C)OC(=O)N1Cc2cn(S(=O)(=O)c3ccc4c(c3)OCCO4)nc2C1 ; O=S(=O)(c1ccc2c(c1)OCCO2)n1cc2c(n1)CNC2]

### Step 1. tert-butyl (3Z)-3-[(dimethylamino)methylidene]-4-oxopyrrolidine-1-carboxylate

To a solution of tert-butyl 3-oxopyrrolidine-1-carboxylate (5 g, 27.0 mmol) in 1,4-dioxane (50 mL) was added (dimethoxymethyl)dimethylamine (7.80 mL, 1.74 mol). The solution was stirred overnight at 100° C. and cooled to room temperature. The resulting mixture was treated with hexane (200 mL). The solids were collected by filtration and dried under vacuum to afford tert-butyl (3Z)-3-[(dimethylamino)methylidene]-4-oxopyrrolidine-1-carboxylate as a yellow solid (3.5 g, 51%). LCMS (ES, m/z): 241 [M+H]⁺.

### Step 2. tert-butyl 2H,4H,5H,6H-pyrrolo[3,4-c]pyrazole-5-carboxylate

To a solution of tert-butyl (3Z)-3-[(dimethylamino)methylidene]-4-oxopyrrolidine-1-carboxylate (2.5 g, 10.40 mmol) in ethanol (20 mL) was added hydrazine (0.65 mL, 20.8 mmol). The resulting solution was stirred for 48 h at 90° C. After cooling to room temperature, the mixture was concentrated under vacuum and washed with hexane to afford tert-butyl 2H,4H,5H,6H-pyrrolo[3,4-c]pyrazole-5-carboxylate as a red oil (620 mg, 28%). LCMS (ES, m/z): 210 [M+H]⁺.

### Step 3. tert-butyl 2-(2,3-dihydro-1,4-benzodioxine-6-sulfonyl)-2H,4H,5H,6H-pyrrolo[3,4-c]pyrazole-5-carboxylate

To a solution of tert-butyl 2H,4H,5H,6H-pyrrolo[3,4-c]pyrazole-5-carboxylate (268 mg, 1.28 mmol) in tetrahydrofuran (6 mL) was added sodium hydride (77 mg, 1.92 mmol, 60% dispersion in mineral oil) in portions at 0° C. The resulting mixture was stirred for 30 min at room temperature. Then 2,3-dihydro-1,4-benzodioxine-6-sulfonyl chloride (300 mg, 1.28 mmol) was added and stirred overnight. The reaction mixture was quenched with water (1 mL) and the product was extracted with ethyl acetate (3×2 mL). The combined organic layers were concentrated under vacuum. The crude product was purified by silica gel chromatography (eluting with 1:1 ethyl acetate/petroleum ether) to afford tert-butyl 2-(2,3-dihydro-1,4-benzodioxine-6-sulfonyl)-2H,4H,5H,6H-pyrrolo[3,4-c]pyrazole-5-carboxylate as a light yellow solid (200 mg, 38%). LCMS (ES, m/z): 408 [M+H]⁺.

### Step 4. tert-butyl 2-(2,3-dihydro-1,4-benzodioxine-6-sulfonyl)-2H,4H,5H,6H-pyrrolo[3,4-c]pyrazole-5-carboxylate

To a solution of tert-butyl 2-(2,3-dihydro-1,4-benzodioxine-6-sulfonyl)-2H,4H,5H,6H-pyrrolo[3,4-c]pyrazole-5-carboxylate (300 mg, 0.74 mmol) in dichloromethane (3 mL) was added trifluoroacetic acid (0.3 mL). The solution was stirred for 2 h at room temperature and concentrated under vacuum. The reaction was rendered basic (pH=8) with saturated sodium bicarbonate solution. The solids were collected by filtration and dried under vacuum to afford 2-(2,3-dihydro-1,4-benzodioxine-6-sulfonyl)-2H,4H,5H,6H-pyrrolo[3,4-c]pyrazole as a white solid (170 mg, 75%). LCMS (ES, m/z): 308 [M+H]⁺.

### Intermediate 73. 2-(difluoromethyl)piperazine HCl Salt

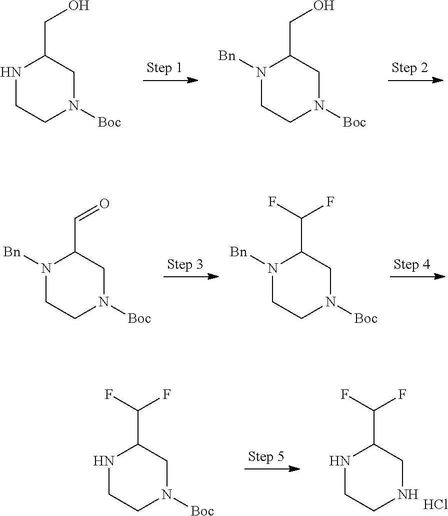

### Step 1. Tert-butyl 4-benzyl-3-(hydroxymethyl)piperazine-1-carboxylate

To a solution of tert-butyl 3-(hydroxymethyl)piperazine-1-carboxylate (7.00 g, 32.4 mmol) in acetonitrile (56 mL) was added TEA (9.00 mL, 64.7 mmol) and (bromomethyl)benzene (5.77 mL, 48.6 mmol). The resulting mixture was stirred for 4 h at 80° C. and then cooled to room temperature. The reaction mixture was concentrated under vacuum. The resulting crude product was purified by silica gel chromatography (eluting with 0:100 to 80:40 ethyl acetate/petroleum ether) to afford tert-butyl 4-benzyl-3-(hydroxymethyl)piperazine-1-carboxylate as a yellow solid (6.60 g, 67%). LCMS (ES, m/z): 307 [M+H]⁺.

### Step 2. Tert-butyl 4-benzyl-3-formylpiperazine-1-carboxylate

To a solution of DMSO (2.29 mL, 32.3 mmol) in DCM (100 mL) was added oxalic dichloride (1.83 mL, 21.5 mmol) dropwise with stirring at −78° C. The reaction was treated with tert-butyl 4-benzyl-3-(hydroxymethyl)piperazine-1-carboxylate (5.50 g, 18.0 mmol) in dichloromethane at −78° C. and stirred for 1 h at −78° C. The solution was treated with TEA (12.5 mL, 89.8 mL) and warmed to room temperature. The reaction mixture was diluted with DCM (100 mL) and then washed with saturated sodium bicarbonate solution (2×100 mL). The organic layer was dried over anhydrous sodium sulfate, filtered and concentrated under vacuum to afford tert-butyl 4-benzyl-3-formylpiperazine-1-carboxylate as a yellow oil (3.5 g, 64%). LCMS (ES, m/z): 305 [M+H]⁺.

### Step 3. Tert-butyl 4-benzyl-3-(difluoromethyl)piperazine-1-carboxylate

To a solution of tert-butyl 4-benzyl-3-formylpiperazine-1-carboxylate (3.50 g, 11.5 mmol) in DCM (80 mL) was added bis(2 methoxyethyl)aminosulfur trifluoride (17 mL, 92.0 mmol) at 0° C. The solution was stirred for 2 h at 0° C. The reaction was treated with ethanol (1.9 mL, 32.7 mmol) and stirred at 0° C. for 1 h. The contents were poured into saturated aqueous sodium bicarbonate solution (200 mL) and extracted with DCM (3×100 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 4-benzyl-3-(difluoromethyl)piperazine-1-carboxylate as a yellow oil (2.0 g, 53%). LCMS (ES, m/z): 327 [M+H]⁺.

### Step 4. Tert-butyl 3-(difluoromethyl)piperazine-1-carboxylate

To a solution of tert-butyl 4-benzyl-3-(difluoromethyl)piperazine-1-carboxylate (500 mg, 1.53 mmol) in methanol (20 mL) was added Pd(OH)₂/C (50 mg, 10 wt % Pd). The resulting mixture was stirred for 2 h at room temperature under hydrogen (2-3 atm). The reaction was purged with nitrogen gas, filtered and concentrated under vacuum to afford tert-butyl 3-(difluoromethyl)piperazine-1-carboxylate as a yellow oil (300 mg, crude). LCMS (ES, m/z): 237 [M+H]⁺.

### Step 5. 2-(difluoromethyl)piperazine HCl Salt

To a stirred solution of tert-butyl 3-(difluoromethyl)piperazine-1-carboxylate (300 mg, 1.28 mmol) in 1,4-dioxane (5 mL) was added hydrochloric acid (5 mL, 3 N). After 4 h, the reaction was concentrated under vacuum to afford 2-(difluoromethyl)piperazine HCl salt as a white solid (300 mg, crude). LCMS (ES, m/z): 137 [M+H]⁺.

### Intermediate 74. 2-acetamido-2-phenylpropanoic Acid

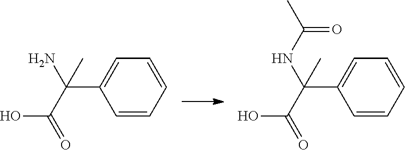

To a solution of 2-amino-2-phenylpropanoic acid (2.00 g, 12.1 mmol) in methanol/water (70 mL, v:v=5:1), was added sodium hydroxide (1.94 g, 48.5 mmol) and acetic anhydride (5.72 mL, 60.5 mmol) at 0° C. The resulting solution was stirred for 1 h at 0° C. The reaction mixture was acidified to pH=5-6 with saturated aqueous citric acid. The resulting solution was extracted with DCM (5×100 mL). The combined organic layers were dried over anhydrous sodium sulfate, filtered, and concentrated under vacuum to afford 2-acetamido-2-phenylpropanoic acid as a light yellow solid (150 mg, 6%). MS (ESI, m/z): 208 [M+H]⁺.

### Intermediate 75. 4-[[(tert-butoxy)carbonyl]amino]-2-(3,5-dichlorophenyl)butanoic Acid

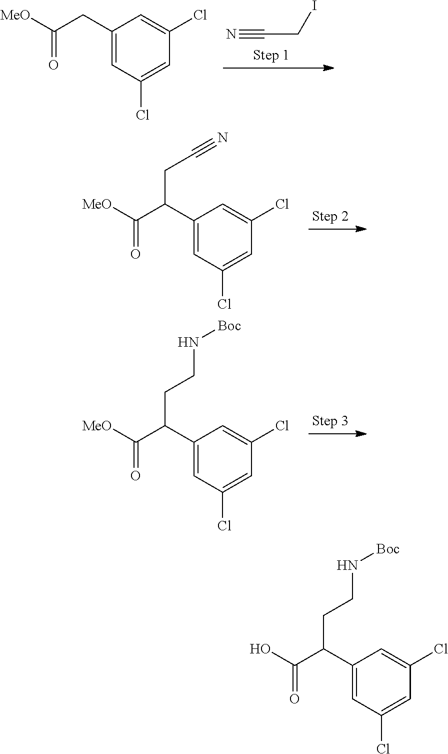

### Step 1. Methyl 3-cyano-2-(3,5-dichlorophenyl)propanoate

To a solution of methyl 2-(3,5-dichlorophenyl)acetate (2.00 g, 8.67 mmol) in tetrahydrofuran (20 mL) was added LDA (5.5 mL, 2 M in THF) dropwise with stirring at −78° C. The solution was stirred for 30 min at −78° C. The reaction was treated with 2-iodoacetonitrile (2.30 g, 13.8 mmol) and stirred for 1 h at −78° C. The contents were poured into saturated aqueous ammonium chloride solution (50 mL) and then extracted with ethyl acetate (3×50 mL). The combined organic layers were dried over anhydrous sodium sulfate, filtered, and concentrated under vacuum. The crude product was purified by silica gel chromatography (eluting with 0:100 to 20:80 ethyl acetate/petroleum ether) to afford methyl 3-cyano-2-(3,5-dichlorophenyl)propanoate as a yellow oil (1.30 g, 58%). LCMS (ES, m/z): 258, 260 [M+H]⁺.

### Step 2. Methyl 4-[[(tert-butoxy)carbonyl]amino]-2-(3,5-dichlorophenyl)butanoate

To a solution of methyl 3-cyano-2-(3,5-dichlorophenyl)propanoate (1.50 g, 5.52 mmol) in methanol (20 mL) was added Raney Ni (946 mg, 11.0 mmol), and di-tert-butyl dicarbonate (6.03 g, 27.6 mmol). The resulting mixture was stirred for 4 h at room temperature under hydrogen (2-3 atm). The reaction mixture was filtered and concentrated under vacuum. The resulting crude product was purified by silica gel chromatography (eluting with 0:100 to 20:80 ethyl acetate/petroleum ether) to afford methyl 4-[[(tert-butoxy)carbonyl]amino]-2-(3,5-dichlorophenyl)butanoate as a yellow oil (1.80 g, 90%). LCMS (ES, m/z): 362, 364 [M+H]⁺.

### Step 3. 4-[[(tert-butoxy)carbonyl]amino]-2-(3,5-dichlorophenyl)butanoic Acid

To a solution of methyl 4-[[(tert-butoxy)carbonyl]amino]-2-(3,5-dichlorophenyl)butanoate (300 mg, 0.79 mmol) in tetrahydrofuran (2 mL) and water (2 mL) was added lithium hydroxide (94 mg, 3.93 mmol). The resulting mixture was stirred for 16 h at room temperature. The reaction mixture was washed with diethyl ether (1×10 mL) and then acidified to pH=7 with saturated aqueous citric acid. The product was extracted with ethyl acetate (3×20 mL). The combined organic layers were dried over anhydrous sodium sulfate, filtered, and concentrated under vacuum to afford 4-[[(tert-butoxy)carbonyl]amino]-2-(3,5-dichlorophenyl)butanoic acid as a yellow oil (180 mg, 66%). LCMS (ES, m/z): 348, 350 [M+H]⁺.

### Intermediate 76. 2-(2-oxopyrrolidin-1-yl)-2-phenylacetic Acid

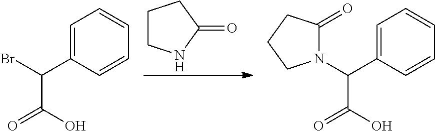

To a solution of pyrrolidin-2-one (800 mg, 9.40 mmol) in toluene (20 mL) was added sodium hydride (820 mg, 20.5 mmol, 60% dispersion in mineral oil) in portions with stirring at 0° C. The resulting mixture was stirred for 1 h at 60° C. A solution of 2-bromo-2-phenylacetic acid (2.00 g, 9.30 mmol) in toluene (20 mL) was added dropwise and stirred for an 1 h at 60° C. The reaction was cooled to room temperature and concentrated under vacuum. The residue was dissolved in water (20 mL) and rendered acidic (pH=2) with hydrochloric acid (6 N). The product was extracted with dichloromethane (5×20 mL). The combined organic layers were dried over anhydrous sodium sulfate, filtered, and concentrated under vacuum. The resulting crude product was purified by Prep-HPLC (XBridge Prep C18 OBD Column, 5 μm, 19×150 mm; Mobile Phase, A: water (containing 5 mmol/L TFA) and B: MeCN (keep 50% B over 40 min); Flow rate: 25 mL/min; Detector: UV 254 nm). The product fractions were concentrated under vacuum to afford 2-(2-oxopyrrolidin-1-yl)-2-phenylacetic acid as a yellow oil (700 mg, 34%). LCMS (ES, m/z): 220 [M+H]⁺.

### Intermediate 77. 2-(3,4-dihydro-2H-benzo[b][1,4]oxazin-6-yl)-2-hydroxyacetic Acid

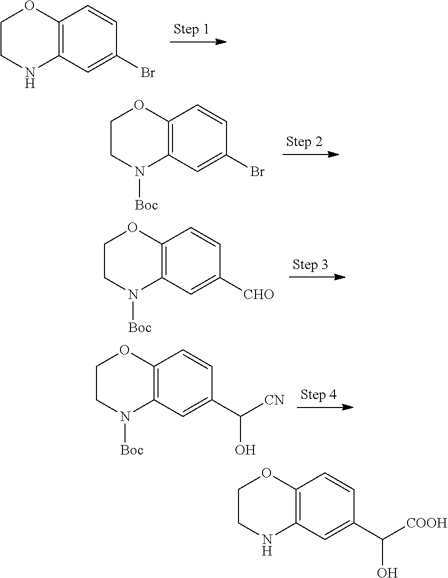

### Step 1. Tert-butyl 6-bromo-2,3-dihydrobenzo[b][1,4]oxazine-4-carboxylate

To a solution of 6-bromo-3,4-dihydro-2H-1,4-benzoxazine (1.40 g, 6.54 mmol) in dichloromethane (10 mL) was added di-tert-butyl dicarbonate (1.71 g, 7.84 mmol), 4-dimethylaminopyridine (80 mg, 0.65 mmol) and TEA (1.81 mL, 13.0 mmol). The solution was stirred overnight at room temperature and concentrated under vacuum. The resulting crude product was purified by prep-TLC (eluting with 1:5 ethyl acetate/petroleum ether) to afford tert-butyl 6-bromo-2,3-dihydrobenzo[b][1,4]oxazine-4-carboxylate as a white solid (1.40 g, 68%). LCMS (ES, m/z): 314, 316 [M+H]⁺.

### Step 2. Tert-butyl 6-formyl-2,3-dihydrobenzo[b][1,4]oxazine-4-carboxylate

To a solution of tert-butyl 6-bromo-2,3-dihydrobenzo[b][1,4]oxazine-4-carboxylate (3.13 g, 9.96 mmol) in tetrahydrofuran (40 mL) was added n-butyllithium (12 mL, 2.50 M in hexane) dropwise with stirring at −78° C. The resulting solution was stirred for 30 min before adding N,N-dimethylformamide (23.8 mL, 308 mmol). The resulting solution was warmed to room temperature and stirred for 1 h. The reaction mixture was poured into water (50 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 6-formyl-2,3-dihydrobenzo[b][1,4]oxazine-4-carboxylate as a light yellow oil (1.80 g, 69%). LCMS (ES, m/z): 264 [M+H]⁺.

### Step 3. Tert-butyl 6-[cyano(hydroxy)methyl]-3,4-dihydro-2H-1,4-benzoxazine-4-carboxylate

To a solution of tert-butyl 6-formyl-2,3-dihydrobenzo[b][1,4]oxazine-4-carboxylate (1.20 g, 4.56 mmol) in dichloromethane (20 mL) was added trimethylsilanecarbonitrile (1.71 mL, 3.00 equiv), and ZnI₂ (145 mg, 0.45 mmol). The mixture was stirred for 1 h at room temperature. The reaction was poured into water (30 mL) and then extracted with ethyl acetate (3×30 mL). The combined organic layers were washed with brine (2×30 mL), dried over anhydrous sodium sulfate, filtered, and concentrated under vacuum to afford tert-butyl 6-[cyano(hydroxy)methyl]-3,4-dihydro-2H-1,4-benzoxazine-4-carboxylate as a yellow oil (900 mg, 68%). LCMS (ES, m/z): 291 [M+H]⁺.

### Step 4. 2-(3,4-dihydro-2H-benzo[b][1,4]oxazin-6-yl)-2-hydroxyacetic Acid

To tert-butyl 6-(cyano(hydroxy)methyl)-2,3-dihydrobenzo[b][1,4]oxazine-4-carboxylate (600 mg, 2.07 mmol) was added hydrochloric acid (8 mL, 6 N). The resulting solution was stirred for 2 h at 60° C. After cooling to room temperature, the mixture was concentrated under vacuum to afford 2-(3,4-dihydro-2H-benzo[b][1,4]oxazin-6-yl)-2-hydroxyacetic acid as a yellow solid (450 mg, 70%). LCMS (ES, m/z): 210 [M+H]⁺.

### Intermediate 78. (2R)-2-(3-bromophenyl)propanoic acid; (2S)-2-(3-bromophenyl)propanoic Acid

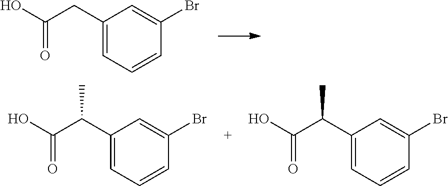

To a solution of 2-(3-bromophenyl)acetic acid (5.00 g, 23.25 mmol) in tetrahydrofuran (100 mL) was added sodium hydride (2.23 g, 55.8 mmol, 60% dispersion in mineral oil) in portions at 0° C. The resulting mixture was stirred for 30 min at 25° C. The reaction was treated with iodomethane (8.68 mL, 139 mmol) at 0° C. and then warmed to room temperature and stirred for 30 min. The reaction mixture was poured into water (25 mL). The resulting solution was acidified to pH=6 with hydrochloric acid (6 N). The product was extracted with ethyl acetate (5×50 mL). The combined organic layers were dried over anhydrous sodium sulfate, filtered and concentrated under vacuum. The resulting crude product was purified by reversed phase chromatography (eluting with 10:90 to 38/62 MeCN/water (containing 0.05% TFA)) to afford 2-(3-bromophenyl)propanoic acid as a yellow oil (3 g, 56%). LCMS (ES, m/z): 227, 229 [M−H]⁻.

The two enantiomers were separated by Prep-SFC (Column: (R,R)-WHELK-O1-Kromasil, 5 μm, 100×460 mm; Mobile Phase, A: CO₂ and B: EtOH (keep 15% B over 6 min); Flow rate: 40 mL/min; Detector: UV 254/220 nm; Retention time: A, 3.72 min; B, 4.23 min). The fraction of the first eluting isomer were collected and concentrated under vacuum to afford (2R)-2-(3-bromophenyl)propanoic acid as a yellow oil (1.4 g, 47%). LCMS (ES, m/z): 227, 229 [M−H]⁻. The fractions of the second eluting isomer were collected and concentrated under vacuum to afford (2S)-2-(3-bromophenyl)propanoic acid as a yellow oil (950 mg, 32%). LCMS (ES, m/z): 227, 229 [M−H]⁻.

### Intermediate 79. Lithium 2-(2-(tert-butoxycarbonyl)isoindolin-4-yl)-2-hydroxyacetate

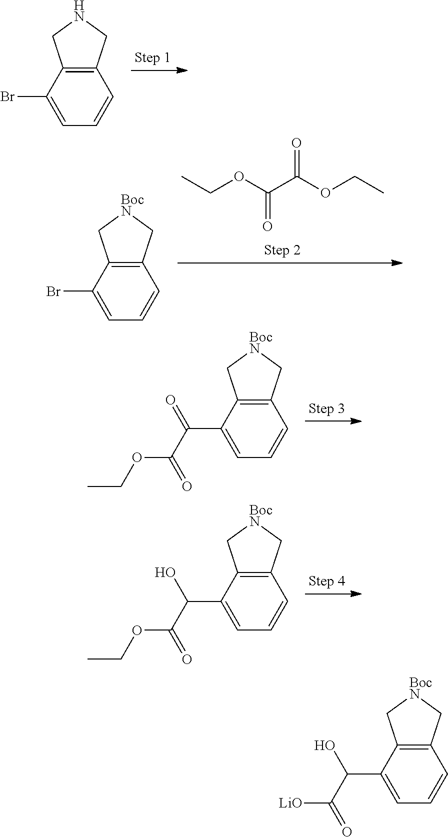

### Step 1. Tert-butyl 4-bromo-2,3-dihydro-1H-isoindole-2-carboxylate

To a solution of 4-bromo-2,3-dihydro-1H-isoindole hydrochloride (4.00 g, 17.1 mmol) in DCM (40 mL) was added triethylamine (7.17 mL, 51.6 mmol) and di-tert-butyl dicarbonate (5.62 g, 25.75 mmol). The resulting solution was stirred for 1 h at room temperature. The reaction mixture was poured into water (40 mL) and then extracted with DCM (3×40 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 4-bromo-2,3-dihydro-1H-isoindole-2-carboxylate as a white solid (5.8 g, 85%). LCMS (ES, m/z): 242, 244 [M+H-t-Bu]⁺.

### Step 2. Tert-butyl 4-(2-ethoxy-2-oxoacetyl)-2,3-dihydro-1H-isoindole-2-carboxylate

To a solution of tert-butyl 4-bromo-2,3-dihydro-1H-isoindole-2-carboxylate (3 g, 10.1 mmol) in THF (100 mL), was added n-BuLi (8 mL, 2.5 moL/L in hexane) at −78° C. After stirring for 1 h at −78° C., diethyl oxalate (2.73 mL, 20.2 mmol) was added dropwise with stirring. The reaction was quenched by pouring the contents into saturated aqueous ammonium chloride (50 mL) and the product was extracted with DCM (5×100 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 4-(2-ethoxy-2-oxoacetyl)-2,3-dihydro-1H-isoindole-2-carboxylate as a yellow oil (0.95 g, 30%). LCMS (ES, m/z): 264 [M+H-t-Bu]⁺.

### Step 3. Tert-butyl 4-(2-ethoxy-1-hydroxy-2-oxoethyl)-2,3-dihydro-1H-isoindole-2-carboxylate

To a solution of tert-butyl 4-(2-ethoxy-2-oxoacetyl)-2,3-dihydro-1H-isoindole-2-carboxylate (650 mg, 2.04 mmol) in THF (10 mL), was added sodium borohydride (23 mg, 0.61 mmol). The resulting mixture was stirred for 10 min at room temperature and concentrated under vacuum to afford tert-butyl 4-(2-ethoxy-1-hydroxy-2-oxoethyl)-2,3-dihydro-1H-isoindole-2-carboxylate as a yellow solid (0.65 g, crude). LCMS (ES, m/z): 266 [M+H-t-Bu]⁺.

### Step 4. Lithium 2-(2-(tert-butoxycarbonyl)isoindolin-4-yl)-2-hydroxyacetate

To a solution of tert-butyl 4-(2-ethoxy-1-hydroxy-2-oxoethyl)-2,3-dihydro-1H-isoindole-2-carboxylate (650 mg, 2.02 mmol) in THF (10 mL) and water (5 mL), was added hydroxide (146 mg, 6.10 mmol). The resulting mixture was stirred for 1 h at room temperature and concentrated under vacuum. The crude product was purified by reversed phase chromatography (eluting with water (containing 10 mmol/L NH₄HCO₃)) to afford lithium 2-(2-(tert-butoxycarbonyl)isoindolin-4-yl)-2-hydroxyacetate as a white solid (0.23 g, 38%). LCMS (ES, m/z): 292 [M-Li⁺]⁻.

### Intermediate 80. Methyl 3-(azetidin-1-yl)-2-(2-methyl-1,3-benzothiazol-4-yl)propanoate

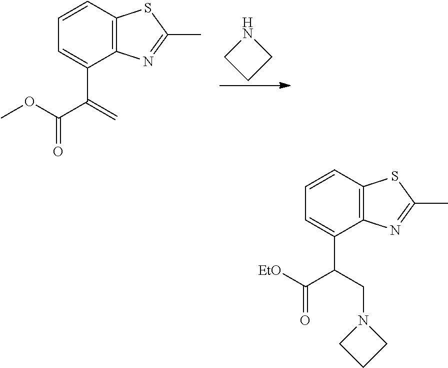

To a solution of methyl 2-(2-methyl-1,3-benzothiazol-4-yl)prop-2-enoate (200 mg, 0.86 mmol) in tetrahydrofuran (2 mL) was added azetidine (98 mg, 1.72 mmol). The resulting mixture was stirred for 2 h at room temperature. The reaction mixture was concentrated under vacuum. The resulting crude product was purified by prep-TLC (eluting with 20:1 dichloromethane/methanol) to afford methyl 3-(azetidin-1-yl)-2-(2-methyl-1,3-benzothiazol-4-yl)propanoate as a brown oil (170 mg, 68%). LCMS (ES, m/z): 305 [M+H]⁺.

### Intermediate 81. 2-hydroxy-2-(1H-indazol-3-yl)acetic Acid

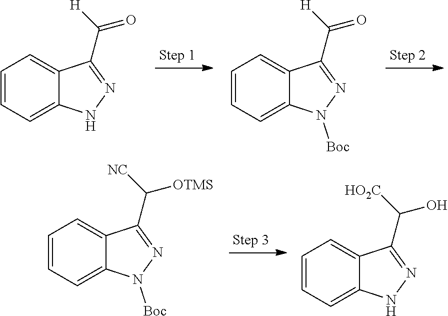

### Step 1. tert-butyl 3-formyl-1H-indazole-1-carboxylate

To a solution of 1H-indazole-3-carbaldehyde (4.0 g, 27.4 mmol) in dichloromethane (50 mL) was added di-tert-butyl dicarbonate (7.17 g, 32.9 mmol), 4-dimethylaminopyridine (334 mg, 2.73 mmol), and TEA (5.70 mL, 41.0 mmol). The resulting solution was stirred for 12 h at room temperature. The reaction mixture was poured into water (100 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 3-formyl-1H-indazole-1-carboxylate as a white solid (5.12 g, 76%). LCMS (ES, m/z): 247 [M+H]⁺.

### Step 2. tert-butyl 3-(cyano((trimethylsilyl)oxy)methyl)-1H-indazole-1-carboxylate

To a solution of tert-butyl 3-formyl-1H-indazole-1-carboxylate (5.10 g, 20.7 mmol) in dichloromethane (80 mL) was added ZnI₂ (660 mg, 2.07 mmol) and trimethylsilanecarbonitrile (9.08 mL, 72.6 mmol) with stirring at 0° C. The resulting solution was warmed to room temperature and stirred for 16 h. The reaction mixture was poured into water (100 mL) and then extracted with ethyl acetate (3×100 mL). The combined organic layers were dried over anhydrous sodium sulfate, filtered, and concentrated under vacuum to afford tert-butyl 3-[cyano(hydroxy)methyl]-1H-indazole-1-carboxylate as a yellow oil (5.00 g, 88%). LCMS (ES, m/z): 346 [M+H]⁺.

### Step 3. 2-hydroxy-2-(1H-indazol-3-yl)acetic Acid

To a solution of tert-butyl 3-[cyano(hydroxy)methyl]-1H-indole-1-carboxylate (1.00 g, 3.67 mmol) in 1,4-dioxane (10 mL) was added hydrochloric acid (10 mL, 6 N). The resulting solution was stirred for 2 h at 80° C. The mixture was concentrated under vacuum to afford 2-hydroxy-2-(1H-indol-3-yl)acetic acid as yellow oil (650 mg, 93%). LCMS (ES, m/z): 193 [M+H]⁺.

### Intermediate 82. 2-{4-[(tert-butoxy)carbonyl]-3,4-dihydro-2H-1,4-benzoxazin-6-yl}-2-acetamidoacetic Acid

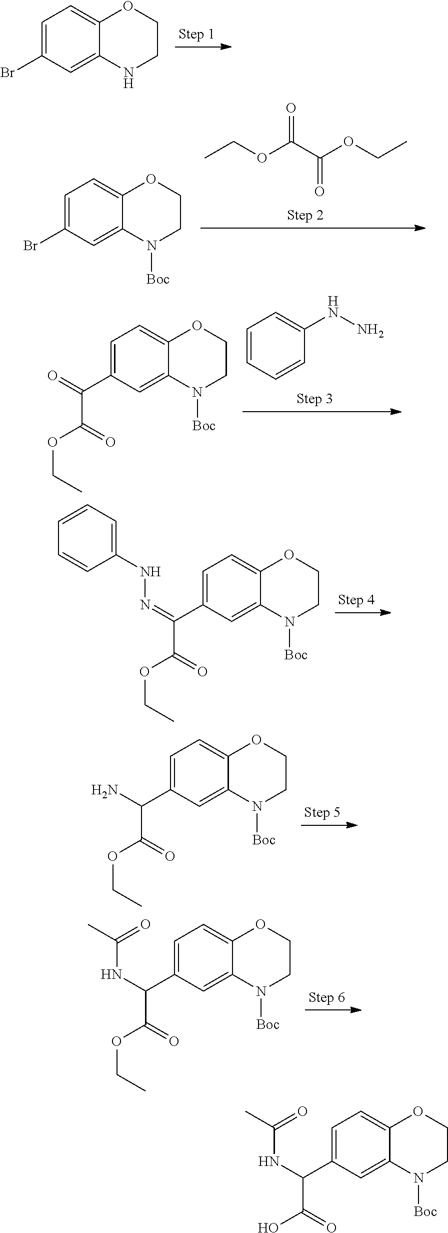

### Step 1. Tert-butyl 6-bromo-3,4-dihydro-2H-1,4-benzoxazine-4-carboxylate

To a solution of 6-bromo-3,4-dihydro-2H-1,4-benzoxazine (3.00 g, 14.0 mmol) in dichloromethane (20 mL) was added triethylamine (11.9 mL, 62.3 mmol) and di-tert-butyl dicarbonate (6.12 g, 28.0 mmol). The solution was stirred for 2 h at 40° C. and concentrated under vacuum. The crude product was purified by silica gel chromatography (eluting with 0:100 to 20:80 ethyl acetate/petroleum ether) to afford tert-butyl 6-bromo-3,4-dihydro-2H-1,4-benzoxazine-4-carboxylate as a yellow oil (3.00 g, 68%). LCMS (ES, m/z): 314, 316 [M+H]⁺.

### Step 2. Tert-butyl 6-(2-ethoxy-2-oxoacetyl)-3,4-dihydro-2H-1,4-benzoxazine-4-carboxylate

To a solution of tert-butyl 6-bromo-3,4-dihydro-2H-1,4-benzoxazine-4-carboxylate (1.00 g, 3.18 mmol) in tetrahydrofuran (30 mL), was added n-butyllithium (1.70 mL, 2.5 M in hexane) dropwise with stirring at −78° C. After 1 h, the reaction was treated with diethyl oxalate (0.98 mL, 6.38 mmol) added dropwise with stirring. After 3 h, the reaction was poured into saturated aqueous ammonium chloride solution (10 mL) and the product was extracted with ethyl acetate (3×20 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 6-(2-ethoxy-2-oxoacetyl)-3,4-dihydro-2H-1,4-benzoxazine-4-carboxylate as a greenish solid (700 mg, 68%). LCMS (ES, m/z): 336 [M+H]⁺.

### Step 3. Tert-butyl 6-[(1E)-2-ethoxy-2-oxo-1-(2-phenylhydrazin-1-ylidene)ethyl]-3,4-dihydro-2H-1,4-benzoxazine-4-carboxylate

To a solution of tert-butyl 6-(2-ethoxy-2-oxoacetyl)-3,4-dihydro-2H-1,4-benzoxazine-4-carboxylate (700 mg, 2.09 mmol) in acetic acid (15 mL) was added phenylhydrazine (554 mg, 5.22 mmol). The resulting mixture was stirred for 1 h at room temperature and concentrated under vacuum. The resulting crude product was purified by silica gel chromatography (eluting with 0:100 to 50:50 ethyl acetate/petroleum ether) to afford 6-[(1E)-2-ethoxy-2-oxo-1-(2-phenylhydrazin-1-ylidene)ethyl]-3,4-dihydro-2H-1,4-benzoxazine-4-carboxylate as a yellow solid (600 mg, 68%). LCMS (ES, m/z): 426 [M+H]⁺.

### Step 4. Tert-butyl 6-(1-amino-2-ethoxy-2-oxoethyl)-3,4-dihydro-2H-1,4-benzoxazine-4-carboxylate

To a solution of 6-[(1E)-2-ethoxy-2-oxo-1-(2-phenylhydrazin-1-ylidene)ethyl]-3,4-dihydro-2H-1,4-benzoxazine-4-carboxylate (500 mg, 1.18 mmol) in acetic acid (10 mL) was added zinc powder (1.15 g, 17.5 mmol) at 0° C. The resulting mixture was stirred overnight at room temperature. The reaction mixture were filtered and concentrated under vacuum. The resulting crude product was purified by reversed phase chromatography (eluting with 0:100 to 40:60 MeCN/water (containing 0.05% NH₄HCO₃)) to afford tert-butyl 6-(1-amino-2-ethoxy-2-oxoethyl)-3,4-dihydro-2H-1,4-benzoxazine-4-carboxylate as a white solid (400 mg, crude). LCMS (ES, m/z): 337 [M+H]⁺.

### Step 5. Tert-butyl 6-(1-acetamido-2-ethoxy-2-oxoethyl)-3,4-dihydro-2H-1,4-benzoxazine-4-carboxylate

To a solution of tert-butyl 6-(1-amino-2-ethoxy-2-oxoethyl)-3,4-dihydro-2H-1,4-benzoxazine-4-carboxylate (400 mg, 1.19 mmol) in dichloromethane (10 mL), was added triethylamine (0.25 mL, 1.80 mmol) and acetyl acetate (0.33 mL, 3.53 mmol). The resulting mixture was stirred for 30 min at room temperature and concentrated under vacuum. The resulting crude product was purified by silica gel chromatography (eluting with 0:100 to 70:30 ethyl acetate/petroleum ether) to afford tert-butyl 6-(1-acetamido-2-ethoxy-2-oxoethyl)-3,4-dihydro-2H-1,4-benzoxazine-4-carboxylate as a white solid (420 mg, 93%). LCMS (ES, m/z): 379 [M+H]⁺.

### Step 6. 2-{4-[(tert-butoxy)carbonyl]-3,4-dihydro-2H-1,4-benzoxazin-6-yl}-2-acetamidoacetic Acid

To a solution of tert-butyl 6-(1-acetamido-2-ethoxy-2-oxoethyl)-3,4-dihydro-2H-1,4-benzoxazine-4-carboxylate (420 mg, 1.11 mmol) in tetrahydrofuran (5 mL) and water (5 mL) was added lithium hydroxide (222 mg, 5.55 mmol). The resulting mixture was stirred overnight at room temperature. The reaction mixture was washed with diethyl ether (1×10 mL) and then acidified to pH=6 with hydrochloric acid (1 N). The resulting solution was extracted with ethyl acetate (3×20 mL). The combined organic layers were dried over anhydrous sodium sulfate, filtered, and concentrated under vacuum to afford 2-[4-[(tert-butoxy)carbonyl]-3,4-dihydro-2H-1,4-benzoxazin-6-yl]-2-acetamidoacetic acid as a yellow solid (370 mg, 95%). LCMS (ES, m/z): 351 [M+H]⁺.

### Intermediate 83. 2-(3-bromophenyl)-2-[(tert-butyldimethylsilyl)oxy]-1-[5-(2,3-dihydro-1,4-benzodioxine-6-sulfonyl)-1H,2H,3H,4H,5H,6H-pyrrolo[3,4-c]pyrrol-2-yl]ethan-1-one

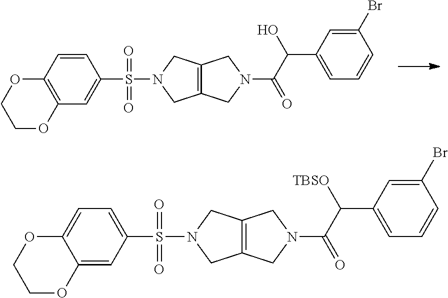 [SMILES: O=C(C(O)c1cccc(Br)c1)N1CC2=C(C1)CN(S(=O)(=O)c1ccc3c(c1)OCCO3)C2 ; CC(C)(C)[Si](C)(C)OC(C(=O)N1CC2=C(C1)CN(S(=O)(=O)c1ccc3c(c1)OCCO3)C2)c1cccc(Br)c1]

To a solution of 2-(3-bromophenyl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (800 mg, 1.53 mmol) in DMF (10 mL) was added imidazole (314 mg, 4.61 mmol), tert-butyl(chloro)dimethylsilane (0.43 mL, 2.30 mmol) and DMAP (20 mg, 0.164 mmol). The resulting solution was stirred for 2 h at 70° C. and then cooled to room temperature. The reaction mixture was poured into water (10 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 2-(3-bromophenyl)-2-[(tert-butyldimethylsilyl)oxy]-1-[5-(2,3-dihydro-1,4-benzodioxine-6-sulfonyl)-1H,2H,3H,4H,5H,6H-pyrrolo[3,4-c]pyrrol-2-yl]ethan-1-one as a light yellow oil (750 mg, 69%). LCMS (ES, m/z): 635, 637 [M+H]⁺.

The Intermediates in Table 19 were synthesized according to the procedure described for Intermediate 83 above.

TABLE 19

| | | LCMS: |
|---|---|---|
| | | (ESI) m/z |
| Intermediate | Structure and Name | [M + H]⁺ |
| 83-2 | 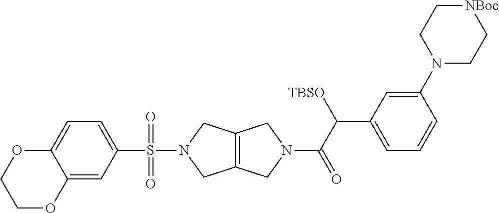 [SMILES: CC(C)(C)OC(=O)N1CCN(c2cccc(C(O[Si](C)(C)C(C)(C)C)C(=O)N3CC4=C(C3)CN(S(=O)(=O)c3ccc5c(c3)OCCO5)C4)c2)CC1] | 741 |
| | tert-butyl 4-(3-(1-((tert-butyldimethylsilyl)oxy)-2-(5-((2,3-dihydrobenzo[b][1,4] | |
| | dioxin-6-yl)sulfonyl)-3,4,5,6-tetrahydropyrrolo[3,4-c]pyrrol-2(1H)-yl)-2- | |
| | oxoethyl)phenyl)piperazine-1-carboxylate | |

### Intermediate 84. Tert-butyl 9,9-difluoro-3,7-diazabicyclo[3.3.1]nonane-3-carboxylate

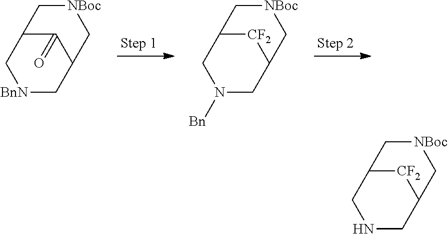 [SMILES: CC(C)(C)OC(=O)N1CC2CN(Cc3ccccc3)CC(C1)C2=O ; CC(C)(C)OC(=O)N1CC2CN(Cc3ccccc3)CC(C1)C2(F)F ; CC(C)(C)OC(=O)N1CC2CNCC(C1)C2(F)F]

### Step 1. Tert-butyl 7-benzyl-9,9-difluoro-3,7-diazabicyclo[3.3.1]nonane-3-carboxylate

To a solution of tert-butyl7-benzyl-9-oxo-3,7-diazabicyclo[3.3.1]nonane-3-carboxylate (5.00 g, 15.1 mmol) in dichloromethane (50 mL), was added DAST (6.0 mL, 45.4 mmol) dropwise with stirring at 0° C. The resulting solution was stirred for 18 h at room temperature. The reaction mixture was poured into ice/water (40 mL) and then extracted with dichloromethane (3×70 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 7-benzyl-9,9-difluoro-3,7-diazabicyclo[3.3.1]nonane-3-carboxylate as a light yellow oil (600 mg, 10%). LCMS (ES, m/z): 353 [M+H]⁺.

### Step 2. Tert-butyl 9,9-difluoro-3,7-diazabicyclo[3.3.1]nonane-3-carboxylate

To a solution of tert-butyl 7-benzyl-9,9-difluoro-3,7-diazabicyclo[3.3.1]nonane-3-carboxylate (600 mg, 1.70 mmol) in MeOH (10 mL), was added Pd(OH)₂/C (60 mg, 20% Pd). The resulting mixture was stirred for 4 h at room temperature under hydrogen atmosphere (2-3 atm). The reaction mixture was filtered and concentrated under vacuum. The resulting crude product was purified by Prep-HPLC (Column: Sunfire Prep C18 OBD Column, 10 μm, 19×250 mm; Mobile Phase A: water (10 MMOL/L NH₄HCO₃), Mobile Phase B: MeCN; Flow rate: 20 mL/min; Gradient: 30% B to 50% B over 7 min; Detector: 254 nm). The fractions of product were concentrated under vacuum to afford tert-butyl 9,9-difluoro-3,7-diazabicyclo[3.3.1]nonane-3-carboxylate as a light yellow solid (360 mg, 72%). LCMS (ES, m/z): 263 [M+H]⁺.

### Intermediate 85. Tert-butyl 7-(3-{2-[5-(2,3-dihydro-1,4-benzodioxine-6-sulfonyl)-1H,2H,3H,4H,5H,6H-pyrrolo [3,4-c]pyrrol-2-yl]-1-hydroxy-2-oxoethyl}phenyl)-9,9-difluoro-3,7-diazabicyclo[3.3.1]nonane-3-carboxylate

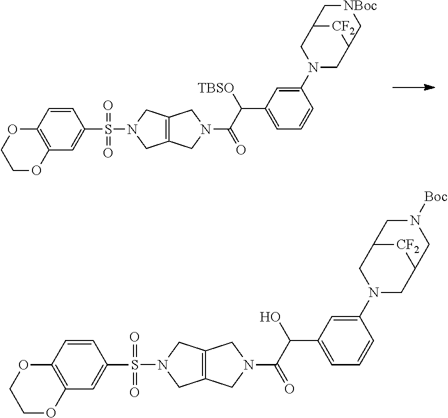 [SMILES: CC(C)(C)OC(=O)N1CC2CN(c3cccc(C(O[Si](C)(C)C(C)(C)C)C(=O)N4CC5=C(C4)CN(S(=O)(=O)c4ccc6c(c4)OCCO6)C5)c3)CC(C1)C2(F)F ; CC(C)(C)OC(=O)N1CC2CN(c3cccc(C(O)C(=O)N4CC5=C(C4)CN(S(=O)(=O)c4ccc6c(c4)OCCO6)C5)c3)CC(C1)C2(F)F]

To 2-[(tert-butyldimethylsilyl)oxy]-2-(3-{9,9-difluoro-3,7-diazabicyclo[3.3.1]nonan-3-yl}phenyl)-1-[5-(2,3-dihydro-1,4-benzodioxine-6-sulfonyl)-1H,2H,3H,4H,5H,6H-pyrrolo[3,4-c]pyrrol-2-yl]ethan-1-one (280 mg, 0.34 mmol) was added TBAF (5 mL, 1 M in THF). The resulting solution was stirred for 30 min at room temperature. The reaction mixture was poured into water (5 mL) and then extracted with ethyl acetate (3×10 mL). The combined organic layers were dried over anhydrous sodium sulfate, filtered, and concentrated under vacuum. The resulting crude product was purified by prep-TLC (eluting with 3:1 ethyl acetate/petroleum ether) to afford tert-butyl 7-(3-[2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]phenyl)-9,9-difluoro-3,7-diazabicyclo[3.3.1]nonane-3-carboxylate as a yellow oil (220 mg, 82%). LCMS (ES, m/z): 703 [M+H]⁺.

The Intermediates in Table 20 were synthesized according to the procedure described for Intermediate 85 above.

TABLE 20

| | | LCMS: |
|---|---|---|
| | | (ESI) m/z |
| Intermediate | Structure and Name | [M + H]⁺ |
| 85-2 | 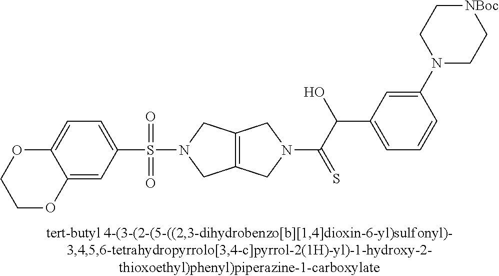 [SMILES: CC(C)(C)OC(=O)N1CCN(c2cccc(C(O)C(=S)N3CC4=C(C3)CN(S(=O)(=O)c3ccc5c(c3)OCCO5)C4)c2)CC1] | 643 |

### Intermediate 86. (3aS,6aS)-3a-fluoro-2-methyl-octahydropyrrolo[3,4-c]pyrrole

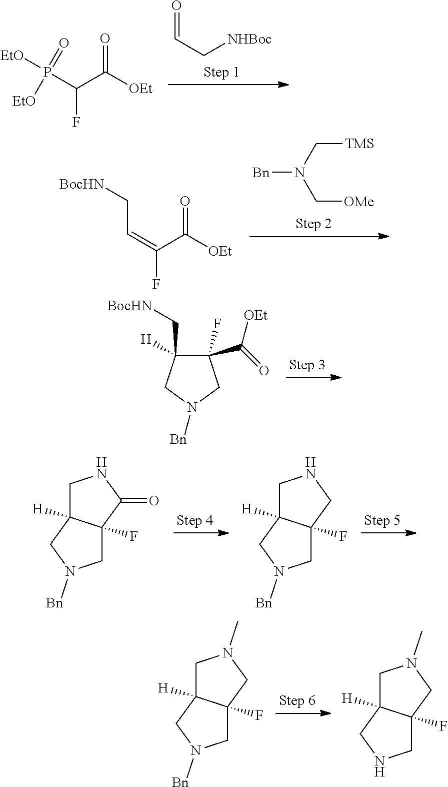

### Step 1. Ethyl (2E)-4-{[(tert-butoxy)carbonyl]amino}-2-fluorobut-2-enoate

To a solution of ethyl 2-(diethoxyphosphoryl)-2-fluoroacetate (10.0 g, 40.5 mmol) in THF (60 mL), was added n-BuLi (16.5 mL, 2.5 M in hexane) dropwise with stirring at −78° C. After 1 h, tert-butyl N-(2-oxoethyl)carbamate (6.57 g, 40.4 mmol) in THF (10 mL) was added. The resulting mixture was stirred for 3 h at −78° C. Then the reaction mixture was poured into saturated sodium bicarbonate (100 mL) and extracted with ethyl acetate (3×100 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford ethyl (2E)-4-{[(tert-butoxy)carbonyl]amino}-2-fluorobut-2-enoate as a light yellow oil (4.1 g, 38%). LCMS (ES, m/z): 248 [M+H]⁺.

### Step 2. Ethyl (3S,4R)-1-benzyl-4-({[(tert-butoxy)carbonyl]amino}methyl)-3-fluoropyrrolidine-3-carboxylate

To a solution of ethyl (2E)-4-[[(tert-butoxy)carbonyl]amino]-2-fluorobut-2-enoate (2.5 g, 10.1 mmol) in DCM (100 mL) was added TFA (0.1 mL) and benzyl(methoxymethyl)[(trimethylsilyl)methyl]amine (2.64 mg, 11.1 mmol). The resulting solution was stirred for 16 h at room temperature and then concentrated under vacuum. The resulting crude product was purified by silica gel chromatography (eluting with 0:100 to 10:90 ethyl acetate/petroleum ether) to afford ethyl (3S,4R)-1-benzyl-4-({[(tert-butoxy)carbonyl]amino}methyl)-3-fluoropyrrolidine-3-carboxylate as a light yellow oil (1.2 g, 31%). LCMS (ES, m/z): 381 [M+H]⁺.

### Step 3. (3aR,6aS)-5-benzyl-6a-fluoro-octahydropyrrolo[3,4-c]pyrrol-1-one

To a solution of ethyl (3S,4R)-1-benzyl-4-({[(tert-butoxy)carbonyl]amino}methyl)-3-fluoropyrrolidine-3-carboxylate (1.20 g, 3.15 mmol) in DCM (20 mL) was added TFA (10 mL). The resulting solution was stirred for 2 h at room temperature and then concentrated under vacuum. The reaction mixture was poured into saturated sodium bicarbonate (100 mL) and then extracted with dichloromethane (3×100 mL). The combined organic layers were dried over anhydrous sodium sulfate, filtered, and concentrated under vacuum to afford (3aR,6aS)-5-benzyl-6a-fluoro-octahydropyrrolo[3,4-c]pyrrol-1-one as a white solid (510 mg, 69%). LCMS (ES, m/z): 235 [M+H]⁺.

### Step 4. (3aR,6aR)-2-benzyl-3a-fluoro-octahydropyrrolo[3,4-c]pyrrole

To a solution of (3aR, 6aS)-5-benzyl-6a-fluoro-octahydropyrrolo[3,4-c]pyrrol-1-one (500 mg, 2.13 mmol) in THF (10 mL) was added borane (10.8 mL, 1 M in THF). The resulting solution was stirred for 16 h at 60° C. The reaction was quenched by the addition of aqueous hydrochloric acid (10 mL, 1.0 N). The resulting mixture was stirred for 2 h at 60° C. and concentrated under vacuum. The resulting crude product was purified by silica gel chromatography (eluting with 0:100 to 10:90 MeOH/DCM) to afford (3aR,6aR)-2-benzyl-3a-fluoro-octahydropyrrolo[3,4-c]pyrrole as light a yellow oil (300 mg, 63%). LCMS (ES, m/z): 221 [M+H]⁺.

### Step 5. (3aR,6aR)-2-benzyl-3a-fluoro-5-methyl-octahydropyrrolo[3,4-c]pyrrole

To a solution of (3aR,6aR)-2-benzyl-3a-fluoro-octahydropyrrolo[3,4-c]pyrrole (300 mg, 1.36 mmol) in MeOH (15 mL) was added formaldehyde (2 mL, 40% in water). The resulting solution was stirred for 30 min followed by addition of STAB (865 mg, 4.09 mmol). The resulting mixture was stirred for 16 h at room temperature and then concentrated under vacuum. The resulting crude product was purified by silica gel chromatography (eluting with 0:100 to 10:90 MeOH/DCM) to afford (3aR,6aR)-2-benzyl-3a-fluoro-5-methyl-octahydropyrrolo[3,4-c]pyrrole as a colorless oil (220 mg, 68%). LCMS (ES, m/z): 235 [M+H]⁺.

### Step 6. (3aS,6aS)-3a-fluoro-2-methyl-octahydropyrrolo[3,4-c]pyrrole

To a solution of (3aR,6aR)-2-benzyl-3a-fluoro-5-methyl-octahydropyrrolo[3,4-c]pyrrole (220 mg, 0.939 mmol) in MeOH (15 mL) was added Pd/C (21.9 mg, 5 wt % Pd). The resulting mixture was stirred for 3 h at 25° C. under hydrogen (2-3 atm). The reaction mixture was filtered and concentrated to afford (3aS,6aS)-3a-fluoro-2-methyl-octahydropyrrolo[3,4-c]pyrrole as light yellow oil (100 mg, 73%). LCMS (ES, m/z): 145 [M+H]⁺.

### Intermediate 87. Tert-butyl 4-(3-{1-[(tert-butyldimethylsilyl)oxy]-2-[5-(2,3-dihydro-1,4-benzodioxine-6-sulfonyl)-1H,2H,3H,4H,5H,6H-pyrrolo[3,4-c]pyrrol-2-yl]-2-sulfanylideneethyl}phenyl)piperazine-1-carboxylate

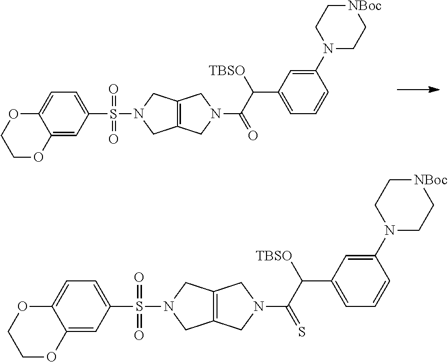 [SMILES: CC(C)(C)OC(=O)N1CCN(c2cccc(C(O[Si](C)(C)C(C)(C)C)C(=O)N3CC4=C(C3)CN(S(=O)(=O)c3ccc5c(c3)OCCO5)C4)c2)CC1 ; CC(C)(C)OC(=O)N1CCN(c2cccc(C(O[Si](C)(C)C(C)(C)C)C(=S)N3CC4=C(C3)CN(S(=O)(=O)c3ccc5c(c3)OCCO5)C4)c2)CC1]

To a solution of tert-butyl 4-(3-[1-[(tert-butyldimethylsilyl)oxy]-2-[5-(2,3-dihydro-1,4-benzodioxine-6-sulfonyl)-1H,2H,3H,4H,5H,6H-pyrrolo[3,4-c]pyrrol-2-yl]-2-oxoethyl]phenyl)piperazine-1-carboxylate (320 mg, 0.43 mmol) in dichloromethane (5 mL), was added Lawesson Reagent (88 mg, 0.22 mmol). The resulting mixture was stirred for 16 h at room temperature. The reaction mixture was concentrated under vacuum. The resulting crude product was purified by Prep-TLC eluting with 1:1 ethyl acetate/petroleum ether to give the product as a light yellow oil (130 mg, 36%). LCMS (ES, m/z) 757 [M+H]⁺.

### Intermediate 88. Tert-butyl 3-(3-bromophenoxy)azetidine-1-carboxylate

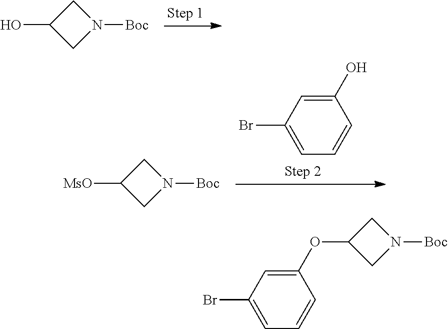 [SMILES: CC(C)(C)OC(=O)N1CC(O)C1 ; Oc1cccc(Br)c1 ; CC(C)(C)OC(=O)N1CC(OS(C)(=O)=O)C1 ; CC(C)(C)OC(=O)N1CC(Oc2cccc(Br)c2)C1]

### Step 1. Tert-butyl 3-(methanesulfonyloxy)azetidine-1-carboxylate

To a solution of tert-butyl 3-hydroxyazetidine-1-carboxylate (5.00 g, 29 mmol) in DCM (200 mL) was added TEA (4.80 mL, 34.8 mmol) and methanesulfonyl chloride (2.50 mL, 31.9 mmol) at 0° C. The resulting solution was stirred for 1 h at 0° C. The reaction was then poured into brine (50 mL) and then extracted with dichloromethane (3×100 mL). The organic layers were combined, dried over anhydrous sodium sulfate, filtered and concentrated under vacuum to afford tert-butyl 3-(methanesulfonyloxy)azetidine-1-carboxylate as a yellow oil (11.1 g, crude). LCMS (ES, m/z): 252 [M+H]⁺.

### Step 2. Tert-butyl 3-(3-bromophenoxy)azetidine-1-carboxylate

To a solution of 3-bromophenol (10.7 g, 61.8 mmol) in DMF (250 mL) was added cesium carbonate (22.5 g, 69.0 mmol) and tert-butyl 3-(methanesulfonyloxy)azetidine-1-carboxylate (11.1 g, 44.2 mmol). The resulting mixture was stirred for 16 h at 80° C. After cooling to room temperature, the reaction mixture was poured into water (250 mL) and then extracted with ethyl acetate (3×50 mL). The combined organic layers were dried over anhydrous sodium sulfate, filtered and concentrated under vacuum. The resulting crude product was purified by silica gel chromatography (eluting with 1:5 ethyl acetate/petroleum ether) to afford tert-butyl 3-(3-bromophenoxy)azetidine-1-carboxylate as a yellow oil (5.58 g, 58%). LCMS (ES, m/z): 328, 330 [M+H]⁺.

### Intermediate 89. 2-[2,3-dihydro(2,2,3,3-2D4)-1,4-benzodioxine-6-sulfonyl]-1H,2H,3H,4H,5H,6H-pyrrolo[3,4-c]pyrrole

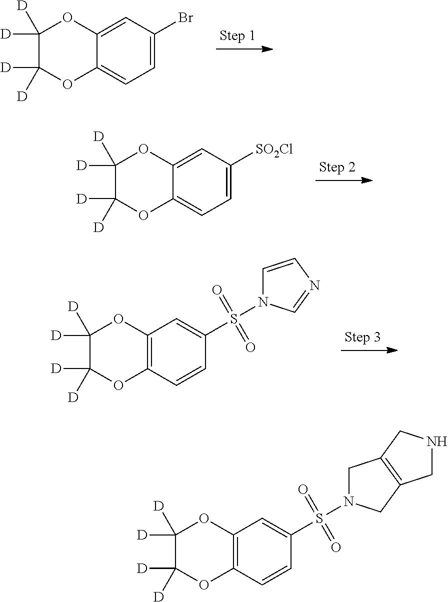 [SMILES: [2H]C1([2H])Oc2ccc(Br)cc2OC1([2H])[2H] ; [2H]C1([2H])Oc2ccc(S(=O)(=O)Cl)cc2OC1([2H])[2H] ; [2H]C1([2H])Oc2ccc(S(=O)(=O)n3ccnc3)cc2OC1([2H])[2H] ; [2H]C1([2H])Oc2ccc(S(=O)(=O)N3CC4=C(CNC4)C3)cc2OC1([2H])[2H]]

### Step 1. 2,3-dihydro(2,2,3,3-2H4)-1,4-benzodioxine-6-sulfonyl Chloride

To a solution of n-BuLi (5.6 mL, 2.5 M in THF) was added n-Bu₂Mg (14 mL, 1 M in THF) at room temperature. The resulting mixture was stirred for 10 min at room temperature and treated with 6-bromo-2,3-dihydro(2,2,3,3-2H4)-1,4-benzodioxine (2.0 g, 9.04 mmol) in tetrahydrofuran (10 mL) added dropwise with stirring at −10° C. The resulting mixture was stirred for 1 h and then added to a solution of sulfuryl chloride (16 mL) in toluene (8 mL) with stirring at −10° C. The resulting mixture was stirred for 0.5 h and quenched with saturated aqueous ammonium chloride solution (30 mL). The product was extracted with ethyl acetate (3×30 mL). The organic layers were combined, dried over anhydrous sodium sulfate, filtered and concentrated under vacuum. The resulting crude product was purified by silica gel chromatography (eluting with 0 to 50% ethyl acetate/petroleum ether) to afford 2,3-dihydro(2,2,3,3-2H4)-1,4-benzodioxine-6-sulfonyl chloride as a yellow oil (1.3 g, 60%).

### Step 2. 1-[2,3-dihydro(2,2,3,3-2H4)-1,4-benzodioxine-6-sulfonyl]-1H-imidazole

To a solution of 2,3-dihydro(2,2,3,3-H)-1,4-benzodioxine-6-sulfonyl chloride (1.3 g, 5.01 mmol) in DCM (20 mL) was added 1H-imidazole (742 mg, 10.9 mmol). The resulting solution was stirred for 2 h at room temperature. Then the reaction mixture was filtered and concentrated under vacuum to afford 1-[2,3-dihydro(2,2,3,3-2H4)-1,4-benzodioxine-6-sulfonyl]-1H-imidazole as a white solid (1.2 g, 89%). LCMS (ES, m/z): 271 [M+H]⁺.

### Step 3. 2-[2,3-dihydro(2,2,3,3-2H4)-1,4-benzodioxine-6-sulfonyl]-1H,2H,3H,4H,5H,6H-pyrrolo[3,4-c]pyrrole

To a solution of 1H,2H,3H,4H,5H,6H-pyrrolo[3,4-c]pyrrole dihydrobromide (3.6 g, 13.2 mmol) in water (15 mL) and ethanol (15 mL) was added 1-[2,3-dihydro(2,2,3,3-2H4)-1,4-benzodioxine-6-sulfonyl]-1H-imidazole (1.2 g, 4.08 mmol). The resulting solution was stirred for 18 h at room temperature and then 48 h at 60° C. After cooling to room temperature, the solution was basified to pH=14 with sodium hydroxide and then extracted with DCM (3×20 mL). The organic layers were combined, dried over anhydrous sodium sulfate, filtered and concentrated under vacuum to afford 2-[2,3-dihydro(2,2,3,3-2H4)-1,4-benzodioxine-6-sulfonyl]-1H,2H,3H,4H,5H,6H-pyrrolo[3,4-c]pyrrole as a yellow solid (500 mg, 39%). LCMS (ES, m/z): 313 [M+H]⁺.

### Intermediate 90. Tert-butyl 5-[(2,2,3,3-²H₄)-2H,3H-[1,4]dioxino[2,3-b]pyridine-7-sulfonyl]-1H,2H,3H,4H,5H,6H-pyrrolo[3,4-c]pyrrole-2-carboxylate

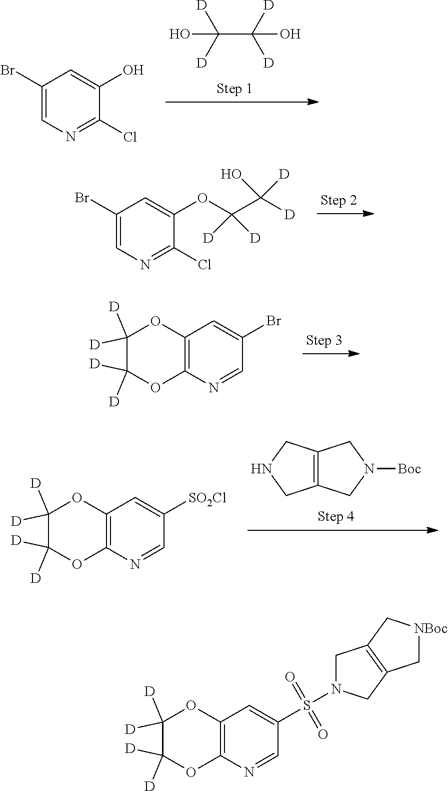

### Step 1. 2-[(5-bromo-2-chloropyridin-3-yl)oxy](²H₄)ethan-1-ol

To a solution of triphenylphosphane (5.7 g, 21.7 mmol) in tetrahydrofuran (90 mL) was added DIAD (4.28 mL, 21.8 mmol) dropwise with stirring at 0° C. The resulting solution was stirred for 30 minutes at room temperature. This was followed by the addition of 5-bromo-2-chloropyridin-3-ol (3 g, 14.1 mmol) and (²H₄)ethane-1,2-diol (957 mg, 14.2 mmol). The resulting solution was stirred overnight at room temperature. The reaction mixture was poured into water (250 mL) and then extracted with ethyl acetate (3×200 mL). The combined organic layers were dried over anhydrous sodium sulfate, filtered and concentrated under vacuum. The resulting crude product was purified by silica gel chromatography (eluting with 1:5 ethyl acetate/petroleum ether) to afford 2-[(5-bromo-2-chloropyridin-3-yl)oxy](²H₄)ethan-1-ol as a light yellow oil (2 g, 55%). LCMS (ES, m/z): 256, 258 [M+H]⁺.

### Step 2. 7-bromo(2,2,3,3-²H₄)-2H,3H-[1,4]dioxino[2,3-b]pyridine

To a solution of 2-[(5-bromo-2-chloropyridin-3-yl)oxy](²H₄)ethan-1-ol (2 g, 7.41 mmol) in ethylene glycol dimethyl ether (20 mL) was added sodium hydride (470 mg, 11.8 mmol, 60% dispersion in mineral oil) in portions at 0° C. The resulting mixture was stirred overnight at 80° C. in an oil bath. After cooling to room temperature, the reaction mixture was poured into water (50 mL) and then extracted with ethyl acetate (3×30 mL). The combined organic layers were dried over anhydrous sodium sulfate, filtered and concentrated under vacuum. The resulting crude product was purified by silica gel chromatography (eluting with 1:5 ethyl acetate/petroleum ether) to afford 7-bromo(2,2,3,3-²H₄)-2H,3H-[1,4]dioxino[2,3-b]pyridine as a light yellow solid (600 mg, 37%). LCMS (ES, m/z): 220, 222 [M+H]⁺.

### Step 3. (2,2,3,3-²H₄)-2H,3H-[1,4]dioxino[2,3-b]pyridine-7-sulfonyl Chloride

To a solution of n-BuLi (2 mL, 2.5 M in heptane) was added n-Bu₂Mg (5 mL, 1.0 M in hexane) at 0° C. The resulting mixture was stirred for 30 min at room temperature. This was followed by addition of a solution of 7-bromo(2,2,3,3-²H₄)-2H,3H-[1,4]dioxino[2,3-b]pyridine (700 mg, 2.86 mmol) in THF (6 mL) dropwise with stirring at −10° C. The resulting mixture was stirred for 2 h at this temperature. A solution of sulfuryl chloride (6 mL) in toluene (6 mL) was added dropwise with stirring at −10° C. After 0.5 h, the mixture was poured into saturated aqueous ammonium chloride solution (20 mL) and then extracted with ethyl acetate (3×15 mL). The combined organic layers were washed with brine (1×15 mL), dried over anhydrous sodium sulfate, filtered and concentrated under vacuum. The resulting crude product was purified by silica gel chromatography (eluting with 0-30% ethyl acetate/petroleum ether) to afford (2,2,3,3-²H₄)-2H,3H-[1,4]dioxino[2,3-b]pyridine-7-sulfonyl chloride as a light yellow oil (200 mg, 29%). LCMS (ES, m/z): 240, 242 [M+H]⁺.

### Step 4. Tert-butyl 5-[(2,2,3,3-²H₄)-2H,3H-[1,4]dioxino[2,3-b]pyridine-7-sulfonyl]-1H,2H,3H,4H,5H,6H-pyrrolo[3,4-c]pyrrole-2-carboxylate

To a solution of (2,2,3,3-2H4)-2H,3H-[1,4]dioxino[2,3-b]pyridine-7-sulfonyl chloride (170 mg, 0.64 mmol) in dichloromethane (3 mL) was added tert-butyl 4,5-dihydropyrrolo[3,4-c]pyrrole-2(1H,3H,4H)-carboxylate (149 mg, 0.67 mmol) and DIEA (0.23 mL, 1.42 mmol). The resulting solution was stirred for 2 h at room temperature. The reaction mixture was poured into water (10 mL) and then extracted with ethyl acetate (3×10 mL). The organic layers were combined, dried over anhydrous sodium sulfate, filtered and concentrated under vacuum. The resulting crude product was purified by silica gel chromatography (eluting with 1:3 ethyl acetate/petroleum ether) to afford tert-butyl 5-[(2,2,3,3-²H₄)-2H,3H-[1,4]dioxino[2,3-b]pyridine-7-sulfonyl]-1H,2H,3H,4H,5H,6H-pyrrolo[3,4-c]pyrrole-2-carboxylate as a yellow solid (200 mg, 76%). LCMS (ES, m/z): 414 [M+H]⁺.

### Intermediate 91. (1S)-2-(5-(2,3-Dihydrobenzo[b][1,4]dioxine-6-sulfonimidoyl)-3,4,5,6-tetrahydropyrrolo[3,4-c]pyrrol-2(1H)-yl)-2-oxo-1-phenylethyl Acetate

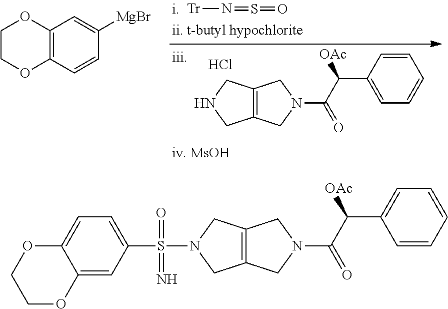

A sample of (2,3-dihydrobenzo[b][1,4]dioxin-6-yl)magnesium bromide (0.5 M in THF) (2.201 ml, 1.100 mmol) in a 2-dram amber vial was cooled on an ice bath, then treated with N-sulfinyltritylamine (339.7 mg, 1.112 mmol). The sample was stirred 5 minutes, then in low light tert-butyl hypochlorite (127.4 mg, 1.173 mmol; caution: fire hazard) was added; the amber vial the hypochlorite was weighed in was rinsed with THF (0.10 mL), and the rinse was added to the rest. The sample was stirred 15 minutes, then (S)-2-oxo-1-phenyl-2-(3,4,5,6-tetrahydropyrrolo[3,4-c]pyrrol-2(1H)-yl)ethyl acetate hydrochloride (359.5 mg, 1.114 mmol) was added. The ice bath was allowed to melt and the sample was stirred at ambient temperature over the weekend. MsOH (0.40 ml, 6.16 mmol) was added and the mixture was triturated. The sample was partitioned between EtOAc and 0.3M aq sodium thiosulfate (20 mL each). The organic layer was dried (Na₂SO₄), filtered, treated with silica gel, and evaporated under reduced pressure. The material was chromatographed by Biotage MPLC (25 g silica gel column, 0 to 100% EtOAc in hexanes, then 1% MeOH in EtOAC) to provide a mixture (˜1:1) of (1S)-2-(5-(2,3-dihydrobenzo[b][1,4]dioxine-6-sulfonimidoyl)-3,4,5,6-tetrahydropyrrolo[3,4-c]pyrrol-2(1H)-yl)-2-oxo-1-phenylethyl acetate and (2S)-1-(5-(2,3-dihydrobenzo[b][1,4]dioxine-6-sulfonimidoyl)-3,4,5,6-tetrahydropyrrolo[3,4-c]pyrrol-2(1H)-yl)-2-hydroxy-2-phenylethan-1-one as a pale orange solid (215 mg). LCMS: Rt 2.15 min., m/z +442.33 [deacetylated product M+H]+; Rt 2.29 min, m/z +484.59 [product, M+H]⁺.

### Methods for Preparing Final Compounds

### Method A

### (2S)-1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one and (2R)-1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one

### Example 94-1. 1^{st} Eluting Isomer; Example 94-2. 2^{nd} Eluting Isomer

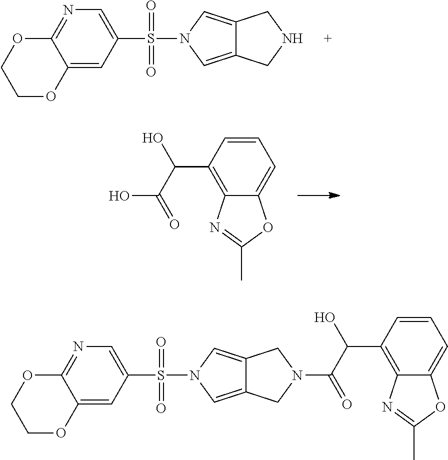 [SMILES: O=S(=O)(c1cnc2c(c1)OCCO2)n1cc2c(c1)CNC2 ; Cc1nc2c(C(O)C(=O)O)cccc2o1 ; Cc1nc2c(C(O)C(=O)N3Cc4cn(S(=O)(=O)c5cnc6c(c5)OCCO6)cc4C3)cccc2o1]

To a solution of 5-[2H,3H-[1,4]dioxino[2,3-b]pyridine-7-sulfonyl]-1H,2H,3H,5H-pyrrolo[3,4-c]pyrrole (180 mg, 0.43 mmol) in N,N-dimethylformamide (10 mL) was added 2-hydroxy-2-(2-methyl-1,3-benzoxazol-4-yl)acetic acid (89 mg, 0.43 mmol), DIEA (110.4 mg, 0.86 mmol), HOBt (63.5 mg, 0.47 mmol) and EDCI (90 mg, 0.47 mmol). The resulting mixture was stirred for 2 h at room temperature and poured into water (50 mL). The resulting solution was extracted with EtOAc (3×30 mL). The combined organic layers were dried over anhydrous sodium sulfate, filtered and concentrated under vacuum. The resulting crude product was purified by prep-TLC (eluting with 1:10 MeOH/DCM). The two enantiomers were separated by Chiral Prep-HPLC (Column: CHIRALPARK IC, 5 μm, 20×250 mm; Mobile Phase, A: DCM and B: MeOH (hold 85% B for 25 min); flow rate: 20 mL/min; Detector: UV 254/220 nm; RT: A (1^{st}), 16.24 min; B (2^{nd}), 21.61 min). The fractions of A were concentrated and lyophilized to afford 1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one, 1^{st} eluting isomer, as a white solid (39.8 mg, 19%). The fractions of B were concentrated and lyophilized to afford 1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one, 2^{nd} eluting isomer, as a white solid (31.7 mg, 15%).

### Method B

### (2R)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one) and (2S)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one)

### Example 95-1. 1st Eluting Isomer; Example 95-2. 2^{nd} Eluting Isomer

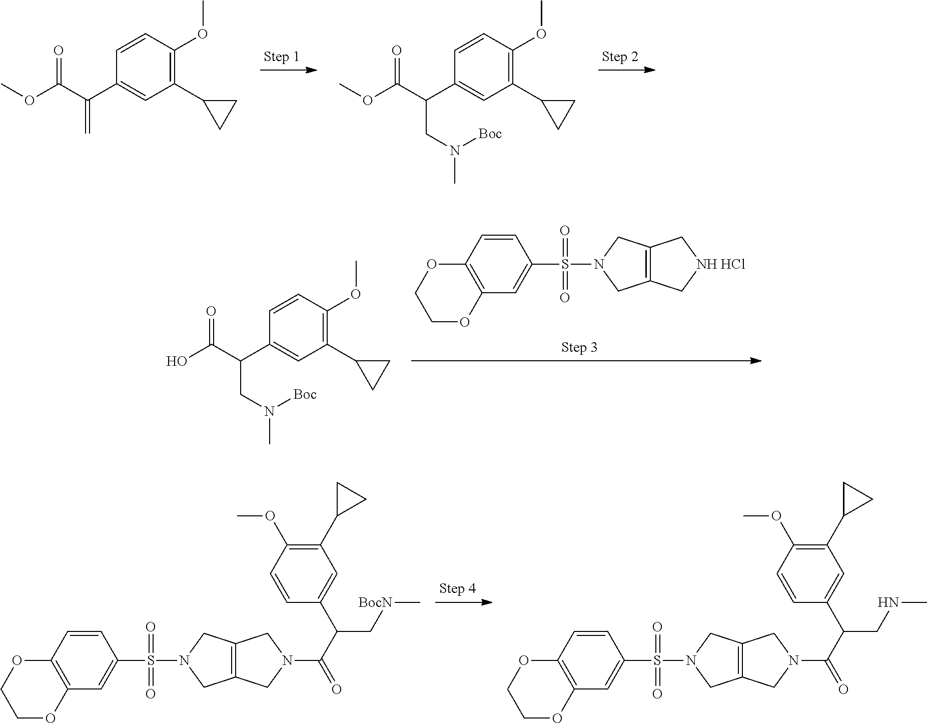

### Step 1. methyl 3-{1[(tert-butoxy)carbonyl](methyl)amino}-2-(3-cyclopropyl-4-methoxyphenyl)propanoate

To a solution of methyl 2-(3-cyclopropyl-4-methoxyphenyl)prop-2-enoate (300 mg, 1.29 mmol) in tetrahydrofuran (10 mL) was added methylamine (2 mL). The resulting mixture was stirred for 30 min at room temperature and concentrated under vacuum to remove excess methylamine. The residue was dissolved in tetrahydrofuran (5 mL) and treated with di-tert-butyl dicarbonate (423 mg, 1.94 mmol). The reaction stirred for 16 h and was concentrated under vacuum. The resulting crude product was purified by silica gel chromatography (eluting with 0:100 to 5:95 ethyl acetate/petroleum ether) to afford methyl 3-{[(tert-butoxy)carbonyl](methyl)amino}-2-(3-cyclopropyl-4-methoxyphenyl)propanoate as a light yellow oil (200 mg, 43%). LCMS (ES, m/z) 364 [M+H]⁺.

### Step 2. 3-{1[(tert-butoxy)carbonyl](methyl)amino}-2-(3-cyclopropyl-4-methoxyphenyl)propanoic Acid

To a solution of methyl 3-{[(tert-butoxy)carbonyl](methyl)amino}-2-(3-cyclopropyl-4-methoxyphenyl)propanoate (200 mg, 0.55 mmol) in tetrahydrofuran (5 mL) was added water (5 mL), and lithium hydroxide (66 mg, 2.75 mmol). The resulting mixture was stirred for 16 h at room temperature. The reaction mixture was washed with diethyl ether (1×5 mL) and then acidified to pH=5 with saturated citric acid. The resulting solution was extracted with ethyl acetate (3×5 mL). The combined organic layers were dried over anhydrous sodium sulfate, filtered, and concentrated under vacuum to afford 3-{[(tert-butoxy)carbonyl](methyl)amino}-2-(3-cyclopropyl-4-methoxyphenyl)propanoic acid as a light yellow oil (150 mg, 87%). LCMS (ES, m/z) 350 [M+H]⁺.

### Step 3. tert-butyl N-[2-(3-cyclopropyl-4-methoxyphenyl)-3-[5-(2,3-dihydro-1,4-benzodioxine-6-sulfonyl)-1H,2H,3H,4H,5H,6H-pyrrolo[3,4-c]pyrrol-2-yl]-3-oxopropyl]-N-methylcarbamate

To a solution of 3-{[(tert-butoxy)carbonyl](methyl)amino}-2-(3-cyclopropyl-4-methoxyphenyl)propanoic acid (150 mg, 0.43 mmol) in DMF (5 mL) was added 2-(2,3-dihydro-1,4-benzodioxine-6-sulfonyl)-1H,2H,3H,4H,5H,6H-pyrrolo[3,4-c]pyrrole (177 mg, 0.51 mmol), DIEA (0.23 mL, 1.29 mmol) and HATU (196 mg, 0.51 mmol). The resulting solution was stirred for 1 h and poured into water (5 mL). The product was extracted with ethyl acetate (3×5 mL). The combined organic layers were dried over anhydrous sodium sulfate, filtered, and concentrated under vacuum. The resulting crude product was purified by prep-TLC (eluting with 1:1 ethyl acetate/petroleum ether) to afford tert-butyl N-[2-(3-cyclopropyl-4-methoxyphenyl)-3-[5-(2,3-dihydro-1,4-benzodioxine-6-sulfonyl)-1H,2H,3H,4H,5H,6H-pyrrolo[3,4-c]pyrrol-2-yl]-3-oxopropyl]-N-methylcarbamate as a light yellow oil (150 mg, 55%). LCMS (ES, m/z) 640 [M+H]⁺.

### Step 4. (2R)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one) and (2S)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one)

To a solution of tert-butyl N-[2-(3-cyclopropyl-4-methoxyphenyl)-3-[5-(2,3-dihydro-1,4-benzodioxine-6-sulfonyl)-1H,2H,3H,4H,5H,6H-pyrrolo[3,4-c]pyrrol-2-yl]-3-oxopropyl]-N-methylcarbamate (150 mg, 0.16 mmol) in dichloromethane (4 mL) was added trifluoroacetic acid (1 mL). The resulting solution was stirred for 2 h at room temperature and concentrated under vacuum. The reaction was quenched with saturated potassium carbonate solution (5 mL) and then extracted with dichloromethane (3×5 mL). The combined organic layers were dried over anhydrous sodium sulfate, filtered, and concentrated under vacuum. The crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (30% to 58% over 7 min); Flow rate: 20 mL/min; Detector: UV 254 nm). The product fractions were concentrated under vacuum. The two enantiomers were further separated by (Column: CHIRALPAK IC, 5 μm, 20×250 mm; Mobile Phase, A: MTBE (containing 0.1% DEA) and B: EtOH (keep 50% B over 18 min); Detector: UV 254/220 nm; Retention time: A (1^{st}), 9.54 min; B (2^{nd}), 12.96 min). The product fractions were concentrated and lyophilized to afford 2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one), 1^{st} eluting isomer, as a white solid (44.3 mg, 70%), and 2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one), 2^{nd} eluting isomer, as a white solid (30.2 mg 48%).

### Method C

### (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one and (2R)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one

### Example 96-1. 1^{st} Eluting Isomer; Example 96-2. 2^{nd} Eluting Isomer

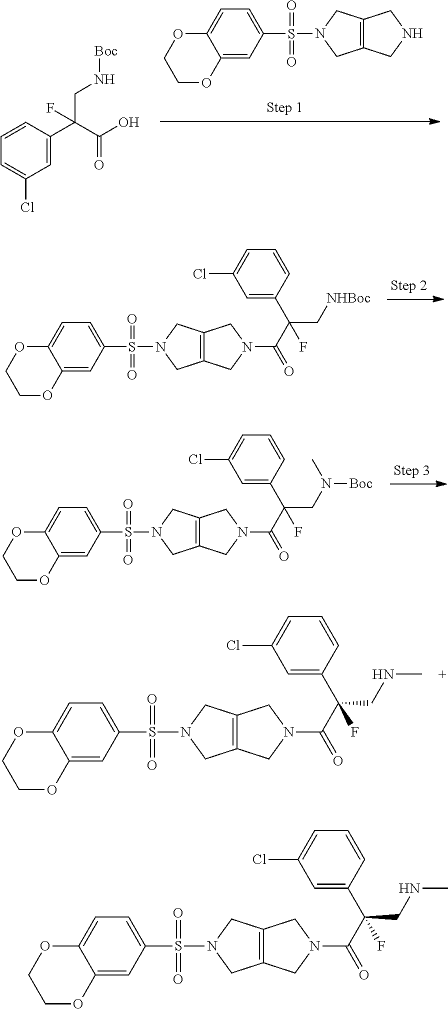 [SMILES: CC(C)(C)OC(=O)NCC(F)(C(=O)O)c1cccc(Cl)c1 ; O=S(=O)(c1ccc2c(c1)OCCO2)N1CC2=C(CNC2)C1 ; CC(C)(C)OC(=O)NCC(F)(C(=O)N1CC2=C(C1)CN(S(=O)(=O)c1ccc3c(c1)OCCO3)C2)c1cccc(Cl)c1 ; CN(CC(F)(C(=O)N1CC2=C(C1)CN(S(=O)(=O)c1ccc3c(c1)OCCO3)C2)c1cccc(Cl)c1)C(=O)OC(C)(C)C ; CNC[C@](F)(C(=O)N1CC2=C(C1)CN(S(=O)(=O)c1ccc3c(c1)OCCO3)C2)c1cccc(Cl)c1 ; CNC[C@@](F)(C(=O)N1CC2=C(C1)CN(S(=O)(=O)c1ccc3c(c1)OCCO3)C2)c1cccc(Cl)c1]

### Step 1. Tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]carbamate

To a solution of 3-[[(tert-butoxy)carbonyl]amino]-2-(3-chlorophenyl)-2-fluoropropanoic acid (120 mg, 0.34 mmol) in DMF (2 mL) was added HATU (155 mg, 0.41 mmol), 2-(2,3-dihydro-1,4-benzodioxine-6-sulfonyl)-1H,2H,3H,4H,5H,6H-pyrrolo[3,4-c]pyrrole (110 mg, 0.34 mmol) and DIEA (132 mg, 1.02 mmol). The resulting solution was stirred for 1 h at room temperature. The reaction mixture was poured into water (10 mL) and then extracted with ethyl acetate (3×20 mL). The combined organic layers were dried over anhydrous sodium sulfate, filtered, and concentrated under vacuum. The resulting crude product was purified by prep-TLC (eluting with 2:1 ethyl acetate/petroleum ether) to afford tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]carbamate as a yellow oil (120 mg, 58%). LCMS (ES, m/z) 608, 610 [M+H]⁺.

### Step 2. Tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]-N-methylcarbamate

To a solution of tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]carbamate (120 mg, 0.20 mmol) in THF (2 mL) was added sodium hydride (10 mg, 0.25 mmol, 60% dispersion in mineral oil) at 0° C. The resulting solution was stirred for 30 min at 0° C. and then treated with iodomethane (28 mg, 0.20 mmol). The resulting mixture was stirred for 6 h at room temperature. The reaction mixture was poured into aqueous ammonium chloride solution (10 mL) and then extracted with ethyl acetate (3×20 mL). The combined organic layers were dried over anhydrous sodium sulfate, filtered, and concentrated under vacuum to afford tert-butyl N-[2-(3-chlorophenyl)-3-[5-(2,3-dihydro-1,4-benzodioxine-6-sulfonyl)-1H,2H,3H,4H,5H,6H-pyrrolo[3,4-c] pyrrol-2-yl]-2-fluoro-3-oxopropyl]-N-methylcarbamate as a yellow oil (120 mg, 98%). LCMS (ES, m/z) 622, 624 [M+H]⁺.

### Step 3. (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one and (2R)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one

To a solution of tert-butyl N-[2-(3-chlorophenyl)-3-[5-(2,3-dihydro-1,4-benzodioxine-6-sulfonyl)-1H,2H,3H,4H,5H,6H-pyrrolo[3,4-c] pyrrol-2-yl]-2-fluoro-3-oxopropyl]-N-methylcarbamate (120 mg, 0.18 mmol) in dichloromethane (2 mL) was added TFA (0.4 mL). The resulting solution was stirred for 1 h at room temperature and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (28% to 50% over 15 min); Flow rate: 20 mL/min; Detector: UV 254 nm). The two enantiomers were further separated by (Column: CHIRALPAK IF, 5 μm, 20×250 mm; Mobile Phase, A: methanol (containing 0.1% DEA) and B: DCM (hold 50% B over 15 min); Detector: UV 254/220 nm; Retention time: A (1^{st}), 8.817 min; B (2^{nd}), 11.059 min). The product fractions of A were concentrated and lyophilized to afford 2-(3-chlorophenyl)-1-[5-(2,3-dihydro-1,4-benzodioxine-6-sulfonyl)-1H,2H,3H,4H,5H,6H-pyrrolo[3,4-c]pyrrol-2-yl]-2-fluoro-3-(methylamino)propan-1-one, 1^{st} eluting isomer, as a white solid (24.4 mg, 26%). The product fractions of B were concentrated and lyophilized to afford 2-(3-chloro-4-cyclopropoxyphenyl)-1-[5-(2,3-Dihydro-1,4-benzodioxine-6-sulfonyl)-1H,2H,3H,4H,5H,6H-pyrrolo[3,4-c]pyrrol-2-yl]-3-(methylamino)propan-1-one, 2^{nd} eluting isomer, as a white solid (11.9 mg, 12%).

### Method D

### (2S)-1-[5-(2,3-dihydro-1,4-benzodioxine-6-sulfonyl)-1H,2H,3H,4H,5H,6H-pyrrolo[3,4-c]pyrrol-2-yl]-2-(7-fluoro-2-methyl-2,3-dihydro-1H-isoindol-4-yl)-2-hydroxyethan-1-one and (2R)-1-[5-(2,3-dihydro-1,4-benzodioxine-6-sulfonyl)-1H,2H,3H,4H,5H,6H-pyrrolo[3,4-c]pyrrol-2-yl]-2-(7-fluoro-2-methyl-2,3-dihydro-1H-isoindol-4-yl)-2-hydroxyethan-1-one

### Example 97-1. 1^{st} Eluting Isomer; Example 97-2. 2^{nd} Eluting Isomer

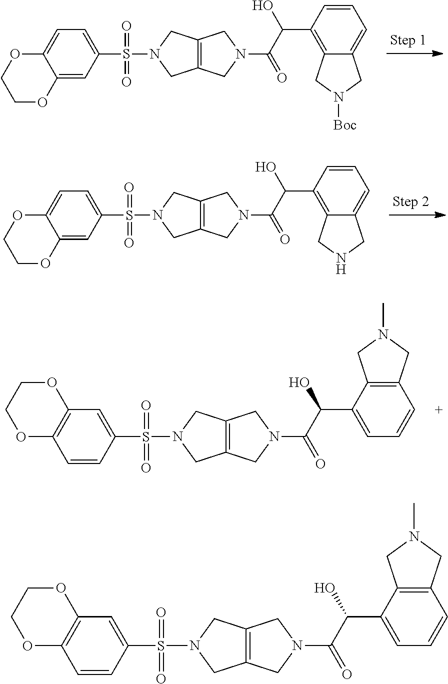

### Step 1. 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)-2-hydroxyethan-1-one

To a solution of tert-butyl 4-[2-[5-(2,3-dihydro-1,4-benzodioxine-6-sulfonyl)-1H,2H,3H,4H,5H,6H-pyrrolo[3,4-c] pyrrol-2-yl]-1-hydroxy-2-oxoethyl]-2,3-dihydro-1H-isoindole-2-carboxylate (780 mg, 1.34 mmol) in dichloromethane (6 mL) was added hydrochloric acid (6 mL, 4 N in 1,4-dioxane). The resulting solution was stirred for 2 h at room temperature and concentrated under vacuum to afford 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)-2-hydroxyethan-1-one HCl salt as a dark red solid (680 mg, 62%). LCMS (ES, m/z) 484 [M+H]⁺.

### Step 2. (2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-2,3-dihydro-1H-isoindol-4-yl)ethan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-2,3-dihydro-1H-isoindol-4-yl)ethan-1-one

To a solution of 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)-2-hydroxyethan-1-one (680 mg, 1.40 mmol) in methanol (7 mL) was added formaldehyde (7 mL, 40 wt % in water). The resulting solution was stirred for 2 h at room temperature and then treated with sodium triacetoxyborohydride (893 mg, 4.21 mmol). The resulting mixture was stirred for 16 h at room temperature and concentrated under vacuum. The crude product was purified by prep-TLC (eluting with 1:10 MeOH/DCM). The enantiomers were separated by prep-Chiral HPLC (Column: CHIRAL ART Cellulose-SB, 5 μm, 20×250 mm; Mobile Phase, A: DCM and B: EtOH (0.1% DEA) (keep 40% B over 10 min); Detector: UV 254/220 nm; Retention time: 1^{st}, 6.63 min; 2^{nd}, 8.63 min. The product fractions were concentrated and lyophilized to afford 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-2,3-dihydro-1H-isoindol-4-yl)ethan-1-one, 1^{st} eluting isomer, as a white solid (41.2 mg, 6%), and 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-methyl-2,3-dihydro-1H-isoindol-4-yl)ethan-1-one, 2^{nd} eluting isomer, as a white solid (42.4 mg, 5%).

### Method E

### (2S)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one and (2R)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one

### Example 98-1. 1^{st} Eluting Isomer; Example 98-2. 2^{nd} Eluting Isomer

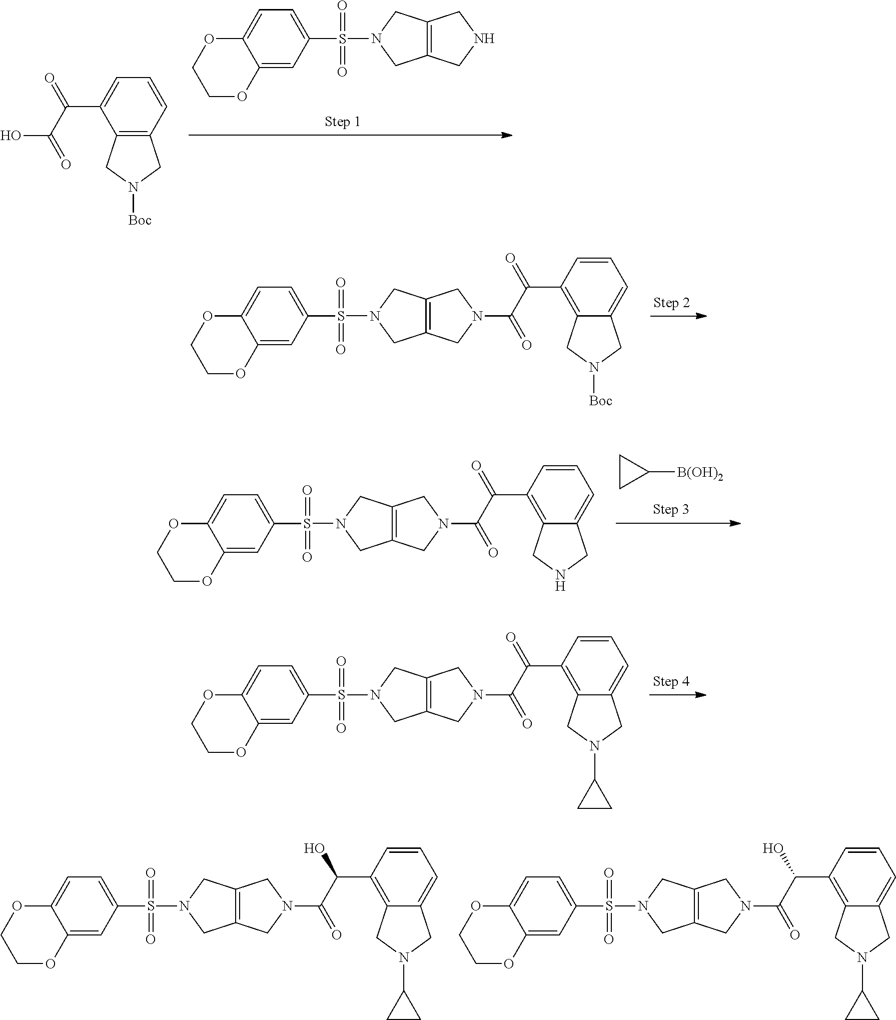

### Step 1. Tert-butyl4-{2-[5-(2,3-dihydro-1,4-benzodioxine-6-sulfonyl)-1H,2H,3H,4H,5H,6H-pyrrolo[3,4-c]pyrrol-2-yl]-2-oxoacetyl}-2,3-dihydro-1H-isoindole-2-carboxylate

To a solution of 2-(2,3-dihydro-1,4-benzodioxine-6-sulfonyl)-1H,2H,3H,4H,5H,6H-pyrrolo [3,4-c]pyrrole (529 mg, 1.63 mmol) in DMF (3 mL) was added 2-[2-[(tert-butoxy)carbonyl]-2,3-dihydro-1H-isoindol-4-yl]-2-oxoacetic acid (500 mg, 1.46 mmol), DIEA (665 mg, 4.89 mmol) and HATU (783 mg, 1.96 mmol). The resulting solution was stirred for 1 h at room temperature. The reaction mixture was poured into water (5 mL) and then extracted with EtOAc (3×5 mL). The combined organic layers were dried over anhydrous sodium sulfate, filtered, and concentrated under vacuum. The resulting crude product was purified by prep-TLC (eluting with 3:1 ethyl acetate/petroleum ether) to afford tert-butyl 4-[2-[5-(2,3-dihydro-1,4-benzodioxine-6-sulfonyl)-1H,2H,3H,4H,5H,6H-pyrrolo[3,4-c]pyrrol-2-yl]-2-oxoacetyl]-2,3-dihydro-1H-isoindole-2-carboxylate as a yellow oil (300 mg, 30%). LCMS (ES, m/z) 582 [M+H]⁺.

### Step 2. 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)ethane-1,2-dione

To a solution of tert-butyl 4-[2-[5-(2,3-dihydro-1,4-benzodioxine-6-sulfonyl)-1H,2H,3H,4H,5H,6H-pyrrolo [3,4-c]pyrrol-2-yl]-2-oxoacetyl]-2,3-dihydro-1H-isoindole-2-carboxylate (300 mg, 0.45 mmol) in DCM (6 mL) was added TFA (1.5 mL). The resulting mixture was stirred for 2 h at room temperature and concentrated under vacuum. The resulting mixture was basified to pH=8 with saturated potassium carbonate solution and then extracted with dichloromethane (2×5 mL). The combined organic layers were dried over anhydrous sodium sulfate, filtered and concentrated under vacuum to afford 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)ethane-1,2-dione as a yellow solid (200 mg, crude). LCMS (ES, m/z) 482 [M+H]⁺.

### Step 3. 1-(2-cyclopropyl-2,3-dihydro-1H-isoindol-4-yl)-2-[5-(2,3-dihydro-1,4-benzodioxine-6-sulfonyl)-1H,2H,3H,4H,5H,6H-pyrrolo[3,4-c]pyrrol-2-yl]ethane-1,2-dione

To a solution of 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)ethane-1,2-dione (217 mg, 0.38 mmol) in 1,2-dichloroethane (15 ml) was added copper (II) acetate (90 mg, 0.43 mmol), 2,2′-bipyridine (70 mg, 0.43 mmol), cyclopropylboronic acid (77 mg, 0.85 mmol) and sodium carbonate (95 mg, 0.86 mmol). The resulting mixture was stirred for 16 h at 70° C. under air atmosphere and cooled to room temperature. The reaction mixture was filtered and poured into water (10 mL). The resulting solution was extracted with ethyl acetate (3×10 mL). The combined organic layers were dried over anhydrous sodium sulfate, filtered and concentrated under vacuum. The resulting crude product was purified by prep-TLC (eluting with 3:1 ethyl acetate/petroleum ether) to afford 1-(2-cyclopropyl-2,3-dihydro-1H-isoindol-4-yl)-2-[5-(2,3-dihydro-1,4-benzodioxine-6-sulfonyl)-1H,2H,3H,4H,5H,6H-pyrrolo[3,4-c]pyrrol-2-yl]ethane-1,2-dione as a white solid (60 mg, 26%). LCMS (ES, m/z) 522 [M+H]⁺.

### Step 4. (2S)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one and (2R)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one

To a solution of 1-(2-cyclopropyl-2,3-dihydro-1H-isoindol-4-yl)-2-[5-(2,3-dihydro-1,4-benzodioxine-6-sulfonyl)-1H,2H,3H,4H,5H,6H-pyrrolo[3,4-c]pyrrol-2-yl]ethane-1,2-dione (60 mg, 0.10 mmol) in methanol (1.5 mL) was added sodium borohydride (9 mg, 0.23 mmol). The resulting solution was stirred for 2 h at room temperature. The reaction mixture was poured into water (5 mL) and then extracted with EtOAc (3×5 mL). The combined organic layers were dried over anhydrous sodium sulfate, filtered and concentrated under vacuum. The resulting crude product was purified by silica gel chromatography (eluting with 10:1 DCM/MeOH) and further purified by Prep-HPLC (Column: XBridge Prep C18 OBD Column (19×150 mm) 5 um; Mobile Phase A: Water (10 mmoL/L NH₄HCO₃), Mobile Phase B: MeCN (30% B to 55% B over 7 min); Flow rate: 20 mL/min; Detector: 254/220 nm). The two enantiomers were further separated by Chiral-Prep-HPLC (Column: CHIRALPAK IE, 2×25 cm, 5 μm; Mobile Phase A: MeOH (containing 0.1% DEA), Mobile Phase B: DCM (Hold 35% B over 14 min); Flow rate: 19 mL/min; Detector: 220/254 nm; A: 9.39 min; B: 12.4 min). The fractions of A were concentrated and lyophilized to afford 2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one, 1^{st} eluting isomer, as a white solid (5.0 mg, 10%). The fractions of B were concentrated and lyophilized to afford 2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one, 2^{nd} eluting isomer, as a white solid (5.3 mg, 10%).

### Method F

### Example 99-1. (2S)-2-(3-chlorophenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one

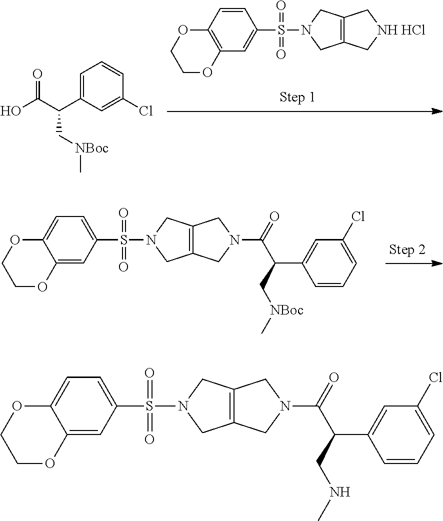

### Step 1. Tert-butyl N-[(2S)-2-(3-chlorophenyl)-3-[5-(2,3-Dihydro-1,4-benzodioxine-6-sulfonyl)1H,2H,3H,4H,5H, 6H-pyrrolo[3,4-c]pyrrol-2-yl]-3-oxopropyl]-N-methylcarbamate

To a solution of (2R)-3-{[(tert-butoxy)carbonyl](methyl)amino}-2-(3-chlorophenyl)propanoic acid (2.30 g, 7.34 mmol) in N,N-dimethylformamide (20 mL) was added HATU (3.07 g, 8.08 mmol), 2-(2,3-dihydro-1,4-benzodioxine-6-sulfonyl)-1H,2H,3H,4H,5H,6H-pyrrolo[3,4-c]pyrrole hydrochloride (2.52 g, 7.34 mmol), and DIEA (3.82 mL, 22.1 mmol). The resulting solution was stirred for 2 h at rt. The reaction mixture was poured into water (100 mL) and then extracted with ethyl acetate (3×100 mL). The combined organic layers were dried over anhydrous sodium sulfate, filtered, and concentrated under vacuum. The crude product was purified by silica gel chromatography (eluting with 1:15 ethyl acetate/dichloromethane) to afford tert-butyl N-[(2S)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate as a white solid (3.5 g, 79%). ¹H-NMR (DMSO-d₆, 400 MHz) δ (ppm): 7.40-7.18 (m, 6H), 7.07-7.06 (m, 1H), 4.45-4.22 (m, 5H), 4.11-3.88 (m, 7H), 3.88-3.58 (m, 2H), 3.44-3.36 (m, 1H), 2.75-2.67 (m, 3H), 1.27-1.16 (m, 9H). LCMS (ES, m/z) 604, 606 [M+H]⁺.

### Step 2. (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-3-(methylamino)propan-1-one

To a solution of tert-butyl N-[(2S)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate (1.5 g, 2.49 mmol) in ethyl acetate (10 mL) was added a solution of hydrochloric acid (10 mL, 4 N in 1,4-dioxane). The resulting solution was stirred for 3 h at 25° C. The mixture was concentrated under vacuum to about ⅓ volume and the solids were collected by filtration. The solids were treated with EtOAc (10 mL) at 70° C., filtered at room temperature, and dissolved with saturated potassium carbonate solution/EA (1:1, 10 mL). The resulting solution was stirred for 3 h and then extracted with EA (3×5 mL). The combined organic layers were dried over anhydrous sodium sulfate, filtered, and concentrated under vacuum to afford (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-3-(methylamino)propan-1-one as a white solid (1 g, 80%). ¹H-NMR (DMSO-d₆, 400 MHz) δ (ppm): 7.36-7.25 (m, 6H), 7.07-7.06 (m, 1H), 4.40-4.29 (m, 5H), 4.06-3.88 (m, 8H), 3.06-3.01 (m, 1H), 2.60-2.50 (m, 1H), 2.23 (s, 3H), 1.66 (s, 1H). LCMS (ES, m/z) 504, 506 [M+H]⁺.

### Method F-1

### (2S)-2-(3-chlorophenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one (Second Eluting Isomer)

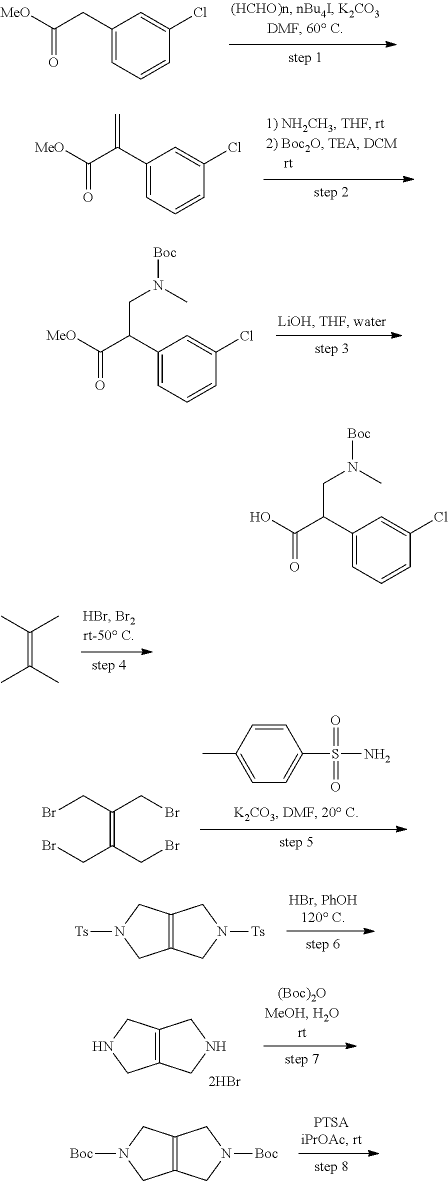

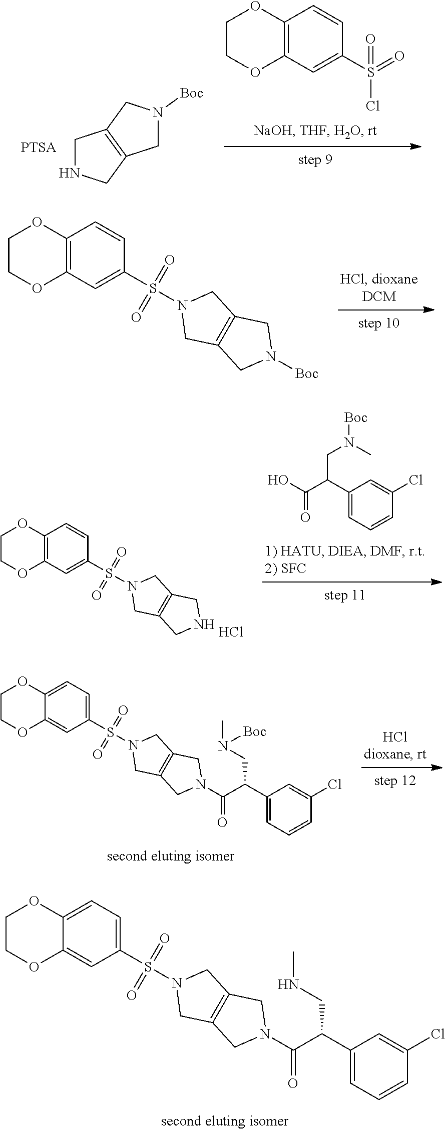

### Step 1. methyl 2-(3-chlorophenyl)prop-2-enoate

To a solution of methyl 2-(3-chlorophenyl)acetate (200 g, 1.09 mol) in N,N-dimethylformamide (1.5 L) was added paraformaldehyde (117 g, 1.30 mol), tetrabutylammonium iodide (40 g, 0.11 mol), and potassium carbonate (373 g, 2.70 mol) successively. The resulting solution was stirred for 10 min at 60° C. and then cooled to rt. The reaction mixture was poured into water (300 mL) and extracted with ethyl acetate (3×500 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford methyl 2-(3-chlorophenyl)prop-2-enoate as colorless oil (60 g, 28%). ¹H-NMR (CDCl₃, 400 MHz) δ (ppm): 7.43 (s, 1H), 7.35-7.27 (m, 3H), 6.43 (s, 1H), 5.94 (s, 1H), 3.94 (s, 3H). LCMS (ES, m/z) 197, 199 [M+H]⁺.

### Step 2. methyl 3-{[(tert-butoxy)carbonyl](methyl)amino}-2-(3-chlorophenyl)propanoate

To a solution of methyl 2-(3-chlorophenyl) prop-2-enoate (60 g, 0.31 mol) in THF (100 mL) was added a solution of methylamine (100 mL, 2 M in THF). The resulting mixture was stirred for 2 h at rt. The resulting solution was concentrated under vacuum, and then dissolved in THF (200 mL). To the above solution was added TEA (84.9 mL, 0.61 mol), and Di-tert-butyl dicarbonate (80.1 g, 0.37 mol). The resulting mixture was stirred for 16 h at rt and then concentrated under vacuum. The resulting crude product was purified by silica gel chromatography (eluting with 0:100 to 5:95 ethyl acetate/petroleum ether) to afford methyl 3-{[(tert-butoxy)carbonyl](methyl)amino}-2-(3-chlorophenyl)propanoate as yellow oil (65 g, 65%). ¹H-NMR (CDCl₃, 400 MHz) δ (ppm): 7.34-7.10 (m, 4H), 3.95-3.87 (m, 1H), 3.78-3.52 (m, 5H), 2.96-2.64 (m, 3H), 1.44 (s, 9H). LCMS (ES, m/z) 328, 330 [M+H]⁺.

### Step 3. 3-{[(tert-butoxy)carbonyl](methyl)amino}-2-(3-chlorophenyl)propanoic Acid

To a solution of methyl 3-{[(tert-butoxy)carbonyl](methyl)amino}-2-(3-chlorophenyl)propanoate (60 g, 0.18 mol) in THF (300 mL) was added water (300 mL), and lithium hydroxide (22 g, 0.91 mol). The resulting mixture was stirred for 16 h at room temperature. The resulting mixture was washed with diethyl ether (1×500 mL), and then acidified to pH=5 with saturated citric acid solution. The resulting solution was extracted with ethyl acetate (3×500 mL). The combined organic layers were dried over anhydrous sodium sulfate, filtered, and concentrated under vacuum to afford 3-{[(tert-butoxy)carbonyl](methyl)amino}-2-(3-chlorophenyl)propanoic acid as yellow oil (23 g, 74%). ¹H-NMR (CD₃OD, 400 MHz) δ (ppm): 7.46-7.19 (m, 4H), 4.05-3.87 (m, 1H), 3.85-3.57 (m, 2H), 2.88-2.69 (m, 3H), 1.44 (s, 9H). LCMS (ES, m/z) 314, 316 [M+H]⁺.

### Step 4. 1,4-dibromo-2,3-bis(bromomethyl)but-2-ene

To a solution of 2,3-dimethylbut-2-ene (1000 g, 11.9 mol) in dichloromethane (500 mL) was slow added a solution of hydrobromic acid (150 mL, 48 wt % in water). This was followed by the addition of bromine (9.90 kg, 61.9 mol) dropwise with stirring at 0-15° C. for about 8 h. The resulting mixture was stirred for 72 h at 45° C. The reaction was then quenched by the careful addition of a saturated aqueous solution of sodium hydrogen sulfite (10 L). The solids formed were collected by filtration, and dried under infrared lamp to afford 1,4-dibromo-2,3-bis(bromomethyl)but-2-ene as a light yellow solid (3.34 kg, 49%). ¹H-NMR (CDCl₃, 400 MHz) δ (ppm): 4.35 (s, 8H). GCMS (EI, m/z): 398, 400, 402 [M]⁺.

### Step 5. 2,5-bis(4-methylbenzenesulfonyl)-1H,2H,3H,4H,5H,6H-pyrrolo[3,4-c]pyrrole

To a solution of 1,4-dibromo-2,3-bis(bromomethyl)but-2-ene (2.00 kg, 3.50 mol) in N,N-dimethylformamide (20 L) was added 4-methylbenzene-1-sulfonamide (2.14 kg, 12.5 mol) and potassium carbonate (5.18 kg, 37.4 mol) successively. The resulting solution was stirred for 48 h at room temperature. The reaction was then quenched by the addition of water/ice (20 L). The solids were then collected by filtration, treated with ethanol (1 L), filtered and dried under vacuum to afford 2,5-bis(4-methylbenzenesulfonyl)-1H,2H,3H,4H,5H,6H-pyrrolo[3,4-c]pyrrole as alight yellow solid (1.34 kg, 78%). ¹H-NMR (DMSO-d₆, 400 MHz) δ (ppm): 7.79-7.62 (m, 4H), 7.49-7.35 (m, 4H), 3.92 (s, 8H), 2.43-2.35 (m, 6H). LCMS (ES, m/z) 419 [M+H]⁺.

### Step 6. 1H,2H,3H,4H,5H,6H-pyrrolo[3,4-c]pyrrole Dihydrobromide

To 2,5-bis(4-methylbenzenesulfonyl)-1H,2H,3H,4H,5H,6H-pyrrolo[3,4-c]pyrrole (1.34 kg, 2.73 mol) was carefully added a solution of hydrobromic acid (4.5 L, 48 wt % in water), and phenol (1.27 kg). The resulting mixture was stirred for 72 h at 120° C. and then cooled to room temperature. The resulting mixture was concentrated under vacuum. The resulting solids were treat with DCM/MeOH (10:1, 1 L), filtered and dried under vacuum to afford 1H,2H,3H,4H,5H,6H-pyrrolo[3,4-c]pyrrole dihydrobromide as a yellow solid (480 g, 55%). ¹H-NMR (D₂O, 400 MHz) δ (ppm): 4.17 (s, 8H). LCMS (ES, m/z) 111 [M+H]⁺.

### Step 7. 2,5-di-tert-butyl 1H,2H,3H,4H,5H,6H-pyrrolo[3,4-c]pyrrole-2,5-dicarboxylate

To a solution of 1H,2H,3H,4H,5H,6H-pyrrolo[3,4-c]pyrrole dihydrobromide (458 g, 1.68 mol) in water (4 L) was added sodium bicarbonate (424 g, 5.05 mol). This was followed by the dropwise addition of a solution of di-tert-butyl dicarbonate (807 g, 3.70 mol) in methanol (500 mL) with stirring at 0° C. The resulting mixture was stirred for 16 h at room temperature. The solids were collected by filtration and dried under vacuum to afford 2,5-di-tert-butyl 1H,2H,3H,4H,5H,6H-pyrrolo[3,4-c]pyrrole-2,5-dicarboxylate as a white solid (300 g, 61%). ¹H-NMR (CDCl₃, 400 MHz) δ (ppm): 4.11 (s, 8H), 1.47 (s, 18H). LCMS (ES, m/z) 296 [M+H-Me]⁺.

### Step 8. Tert-butyl 1H,2H,3H,4H,5H,6H-pyrrolo[3,4-c]pyrrole-2-carboxylate PTSA Salt

To a solution of 2,5-di-tert-butyl 1H,2H,3H,4H,5H,6H-pyrrolo[3,4-c]pyrrole-2,5-dicarboxylate (200 g, 0.65 mol) in propan-2-yl acetate (5 L) was added 4-methylbenzene-1-sulfonic acid (123 g, 0.65 mol). The resulting mixture was stirred for 16 h at 55° C. and then cooled to room temperature. The solids were collected by filtration and dried under vacuum to afford tert-butyl 1H,2H,3H,4H,5H,6H-pyrrolo[3,4-c]pyrrole-2-carboxylate PTSA salt as a yellow solid (197 g, 80%). ¹H-NMR (CD₃OD, 400 MHz) δ (ppm): 7.73-7.71 (d, J=8.0 Hz, 2H), 7.27-7.25 (d, J=8.0 Hz, 2H), 4.09 (s, 8H), 2.39 (s, 3H), 1.51 (s, 9H). LCMS (ES, m/z) 211 [M+H]⁺.

### Step 9. Tert-butyl 5-(2,3-dihydro-1,4-benzodioxine-6-sulfonyl)-1H,2H,3H,4H,5H,6H-pyrrolo[3,4-c]pyrrole-2-carboxylate

To a mixture of tert-butyl 1H,2H,3H,4H,5H,6H-pyrrolo[3,4-c]pyrrole-2-carboxylate PTSA salt (61 g, 0.16 mol) in THF (90 mL) was added a solution of sodium hydroxide (13 g, 0.32 mol) in water (300 mL), and then 2,3-dihydro-1,4-benzodioxine-6-sulfonyl chloride (25 g, 0.11 mol). The resulting mixture was stirred for 3 h at room temperature. The reaction mixture was treated with water (200 mL) and extracted with ethyl acetate (3×400 mL). The combined organic layers were dried over anhydrous sodium sulfate, filtered, and concentrated under vacuum. The resulting crude product was purified by silica gel chromatography (eluting with 0:100 to 10:90 ethyl acetate/dichloromethane) to afford tert-butyl 5-(2,3-dihydro-1,4-benzodioxine-6-sulfonyl)-1H,2H,3H,4H,5H,6H-pyrrolo[3,4-c]pyrrole-2-carboxylate (30 g, 46%). ¹H-NMR (CDCl₃, 400 MHz) δ (ppm): 7.37-7.28 (m, 2H), 6.99-6.97 (m, 1H), 4.34-4.29 (m, 4H), 4.09-4.01 (m, 8H), 1.45 (s, 9H). LCMS (ES, m/z) 409 [M+H]⁺.

### Step 10. 2-(2,3-dihydro-1,4-benzodioxine-6-sulfonyl)-1H,2H,3H,4H,5H,6H-pyrrolo[3,4-c]pyrrole Hydrochloride

To a solution of tert-butyl 5-(2,3-dihydro-1,4-benzodioxine-6-sulfonyl)-1H,2H,3H,4H,5H,6H-pyrrolo[3,4-c]pyrrole-2-carboxylate (30 g, 0.69 mol) in dichloromethane (100 mL) was added a solution of hydrochloric acid (200 mL, 4 N in 1,4-dioxane). The resulting solution was stirred for 2 h at room temperature and then concentrated under vacuum to afford 2-(2,3-dihydro-1,4-benzodioxine-6-sulfonyl)-1H,2H,3H,4H,5H,6H-pyrrolo[3,4-c]pyrrole hydrochloride as a yellow solid (20 g, 79%). ¹H-NMR (DMSO-d₆, 400 MHz) δ (ppm): 10.29 (s, 2H), 7.32-7.22 (m, 2H), 7.11-7.05 (m, 1H), 4.36-4.32 (m, 4H), 4.04 (s, 4H), 3.94-3.87 (m, 4H). LCMS (ES, m/z) 309 [M+H]⁺.

### Step 11. Tert-butyl N-[(2S)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate

To a solution of 3-{[(tert-butoxy)carbonyl](methyl)amino}-2-(3-chlorophenyl)propanoic acid (50.9 g, 0.16 mol) in N,N-dimethylformamide (1 L) was added HATU (57.4 g, 0.15 mol), 2-(2,3-dihydro-1,4-benzodioxine-6-sulfonyl)-1H,2H,3H,4H,5H,6H-pyrrolo[3,4-c]pyrrole hydrochloride (40 g, 0.11 mol), and DIEA (40 mL, 0.23 mol). The resulting solution was stirred for 1 h at rt. The reaction mixture was poured into water (1 L) and then extracted with ethyl acetate (3×1.5 L). The combined organic layers were dried over anhydrous sodium sulfate, filtered, and concentrated under vacuum. The crude product was purified by silica gel chromatography (eluting with 10:90 ethyl acetate/dichloromethane). The two enantiomers were further separated by SFC with the following conditions: Column: Enantiocel-C1, 5 μm, 19×150 mm; Mobile Phase, A: CO₂ and B: MeOH (containing 2 mM NH₃) (hold 50% B for 10 min); flow rate: 180 mL/min; Detector: UV 254 nm; Rt: A, 6.02 min; B, 7.66 min). Then the fractions of B were concentrated and lyophilized to afford tert-butyl N-[(2S)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate as a white solid (14 g, 21%). ¹H-NMR (DMSO-d₆, 400 MHz) δ (ppm): 7.40-7.18 (m, 6H), 7.07-7.06 (m, 1H), 4.45-4.22 (m, 5H), 4.11-3.88 (m, 7H), 3.88-3.58 (m, 2H), 3.44-3.36 (m, 1H), 2.75-2.67 (m, 3H), 1.27-1.16 (m, 9H). LCMS (ES, m/z) 604, 606 [M+H]⁺.

### Step 12. (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-3-(methylamino)propan-1-one

To a solution of tert-butyl N-[(2S)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate (3.0 g, 4.92 mmol) in DCM (80 mL) was added a solution of hydrochloric acid (10 mL, 4 N in 1,4-dioxane). The resulting solution was stirred for 4 h at 25° C. The resulting mixture was concentrated under vacuum to about ⅓ volume. The solids formed were collected by filtration, washed with EA (100 mL), and then dissolved with saturated potassium carbonate solution/EA (1:1, 200 mL). The resulting solution was stirred for 3 h and then extracted with EA (3×50 mL). The combined organic layers were dried over anhydrous sodium sulfate, filtered, and concentrated under vacuum to afford (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-3-(methylamino)propan-1-one as a white solid (1.9 g, 75.9%). ¹H-NMR (DMSO-d₆, 400 MHz) δ (ppm): 7.36-7.25 (m, 6H), 7.07-7.05 (m, 1H), 4.42-4.37 (m, 1H), 4.35-4.29 (m, 4H), 4.06-3.88 (m, 8H), 3.06-3.01 (m, 1H), 2.60-2.55 (m, 1H), 2.23 (s, 3H), 1.66 (s, 1H). LCMS (ES, m/z) 504, 506 [M+H]⁺.

### Method G

### (2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,2,3,4-tetrahydroisoquinolin-5-yl)ethan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,2,3,4-tetrahydroisoquinolin-5-yl)ethan-1-one

### Example 100-1. 1^{st} Eluting Isomer; Example 100-2. 2^{nd} Eluting Isomer

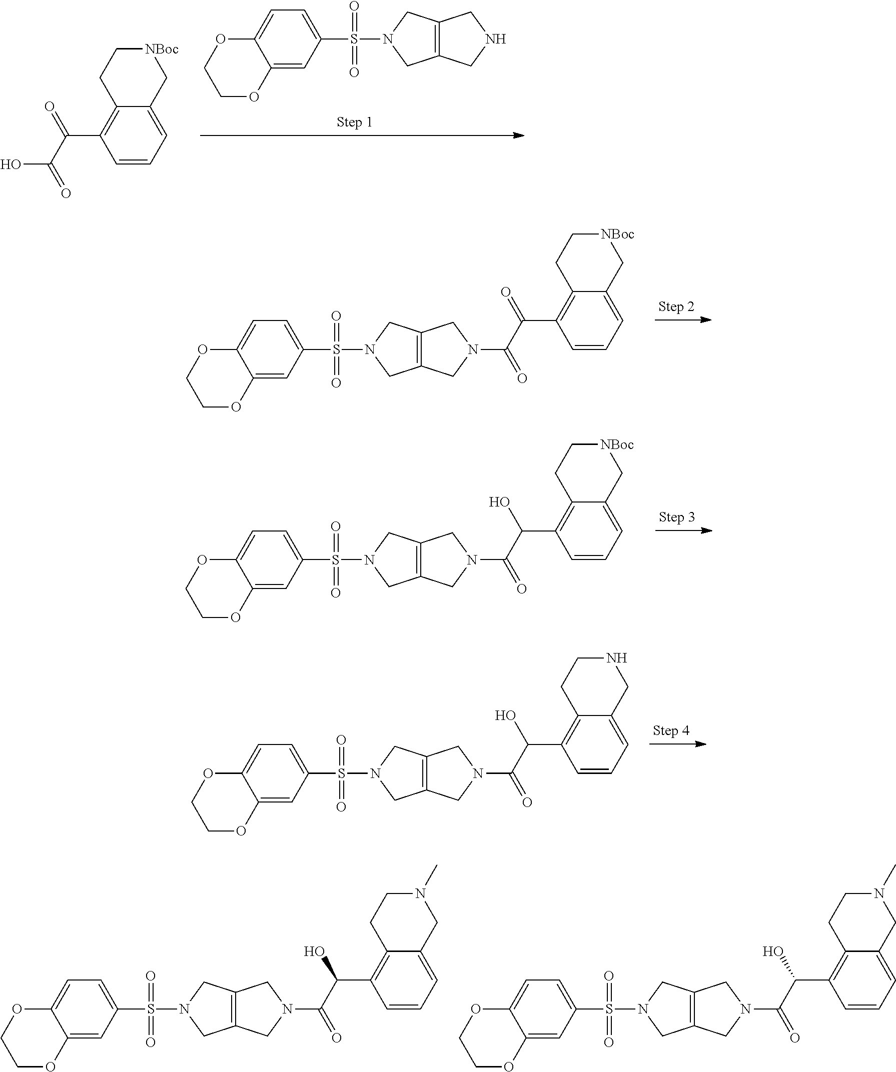

### Step 1. tert-Butyl 5-[2-[5-(2,3-dihydro-1,4-benzodioxine-6-sulfonyl)-1H,2H,3H,4H,5H,6H-pyrrolo[3,4-c]pyrrol-2-yl]-2-oxoacetyl]-1,2,3,4-tetrahydroisoquinoline-2-carboxylate

To a solution of 2-[2-[(tert-butoxy)carbonyl]-1,2,3,4-tetrahydroisoquinolin-5-yl]-2-oxoacetic acid (200 mg, 0.66 mmol) in DMF (3 mL) was added 2-(2,3-dihydro-1,4-benzodioxine-6-sulfonyl)-1H,2H,3H,4H,5H,6H-pyrrolo[3,4-c]pyrrole (242 mg, 0.78 mmol), DIEA (0.33 mL, 1.96 mmol), and HATU (298 mg, 0.78 mmol). The resulting solution was stirred for 1 h at room temperature. The reaction mixture was poured into water (20 mL) and then extracted with EtOAc (4×50 mL). The combined organic layers were dried over anhydrous sodium sulfate, filtered, and concentrated under vacuum. The resulting crude product was purified by prep-TLC (eluting with 2:1 ethyl acetate/petroleum ether) to afford tert-butyl 5-[2-[5-(2,3-dihydro-1,4-benzodioxine-6-sulfonyl)-1H,2H,3H,4H,5H,6H-pyrrolo[3,4-c]pyrrol-2-yl]-2-oxoacetyl]-1,2,3,4-tetrahydroisoquinoline-2-carboxylate as a white solid (180 mg, 39%). LCMS (ES, m/z): 596 [M+H]⁺.

### Step 2. tert-butyl 5-[2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]-1,2,3,4-tetrahydroisoquinoline-2-carboxylate

To a solution of tert-butyl 5-[2-[5-(2,3-dihydro-1,4-benzodioxine-6-sulfonyl)-1H,2H,3H,4H,5H,6H-pyrrolo[3,4-c]pyrrol-2-yl]-2-oxoacetyl]-1,2,3,4-tetrahydroisoquinoline-2-carboxylate (180 mg, 0.30 mmol) in methanol (3 mL) was added sodium borohydride (23 mg, 0.61 mmol). The resulting solution was stirred for 1 h at rt. The mixture was concentrated under vacuum and treated with water (20 mL) and then extracted with EtOAc (3×50 mL). The combined organic layers were dried over anhydrous sodium sulfate, filtered, and concentrated under vacuum. The resulting crude product was purified by Prep-TLC (eluting with 5:1 ethyl acetate/petroleum ether) to afford tert-butyl 5-[2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]-1,2,3,4-tetrahydroisoquinoline-2-carboxylate as a white solid (120 mg, 66%). LCMS (ES, m/z): 598 [M+H]⁺.

### Step 3. 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(1,2,3,4-tetrahydroisoquinolin-5-yl)ethan-1-one

To a solution of tert-butyl 5-[2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]-1,2,3,4-tetrahydroisoquinoline-2-carboxylate (120 mg, 0.20 mmol) in dichloromethane (1 mL) was added trifluoroacetic acid (4 mL). The resulting solution was stirred for 2 h at room temperature and concentrated under vacuum. The resulting mixture was basified to pH=8 with saturated potassium carbonate solution and then extracted with dichloromethane (2×5 mL). The combined organic layers were dried over anhydrous sodium sulfate, filtered, and concentrated under vacuum to afford 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(1,2,3,4-tetrahydroisoquinolin-5-yl)ethan-1-one as a white solid (100 mg, 60%). LCMS (ES, m/z): 498 [M+H]⁺.

### Step 4. (2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,2,3,4-tetrahydroisoquinolin-5-yl)ethan-1-one and (2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,2,3,4-tetrahydroisoquinolin-5-yl)ethan-1-one

To a solution of 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(1,2,3,4-tetrahydroisoquinolin-5-yl)ethan-1-one (100 mg, 0.20 mmol) in dichloromethane (3 mL) was added paraformaldehyde (30 mg, 1.00 mmol) and trifluoroacetic acid (20 mg). The resulting solution was stirred for 1 h at rt. To the reaction was added sodium triacetoxyborohyride (212 mg, 1.00 mmol) was added stirred for 16 h. The resulting mixture was concentrated under vacuum, treated with water (20 mL) and then extracted with EtOAc (3×50 mL). The combined organic layers were dried over anhydrous sodium sulfate, filtered, and concentrated under vacuum. The crude product was purified by Prep-HPLC (Column: XBridge Prep C18 OBD Column 19×150 mm, 5 μm; Mobile Phase A: water (10 mmoL/L NH₄HCO₃), Mobile Phase B: MeCN (28% B to 48% B over 7 min); Flow rate: 20 mL/min; Detector: 254/220 nm). The product fractions were concentrated under vacuum. The two enantiomers were further separated by Prep-Chiral HPLC (Column: CHIRAL ART Cellulose-SB, 2×25 cm, 5 μm; Mobile Phase A: MTBE (0.1% DEA), Mobile Phase B: MeOH (keep 50% B over 23 min); Flow rate: 20 mL/min; Detector: 220/254 nm; 1^{st}: 12.0 min; 2^{nd}: 18.9 min). The product fractions were concentrated and lyophilized to afford 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,2,3,4-tetrahydroisoquinolin-5-yl)ethan-1-one, 1^{st} eluting isomer, as a white solid (3.8 mg, 1%), and 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,2,3,4-tetrahydroisoquinolin-5-yl)ethan-1-one, 2^{nd} eluting isomer, as a white solid (3 mg, 1%).

### Method H

### (2R)-1-[5-(2,3-dihydro-1,4-benzodioxine-6-sulfonyl)-1H,2H,3H,4H,5H,6H-pyrrolo[3,4-c]pyrrol-2-yl]-3-(dimethylamino)-2-(5-fluoro-2-methoxyphenyl)propan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-3-(dimethylamino)-2-(5-fluoro-2-methoxyphenyl)propan-1-one

### Example 101-1. 1^{st} Eluting Isomer; Example 101-2. 2^{nd} Eluting Isomer

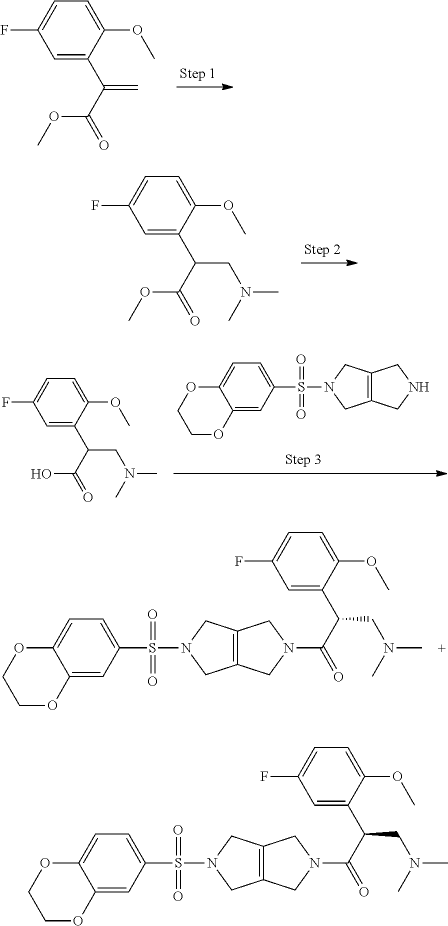

### Step 1. Methyl 2-(dimethylamino)-2-(4-fluoro-2-methoxyphenyl)acetate

To a solution of dimethylamine (10 mL, 2 M in THF) was added methyl 2-(4-fluoro-2-methoxyphenyl)prop-2-enoate (500 mg, 2.02 mmol). The resulting mixture was stirred for 4 h at rt and then concentrated under vacuum. The resulting crude product was purified by silica gel chromatography (eluting with 1:1 ethyl acetate/petroleum ether) to afford methyl 2-(dimethylamino)-2-(4-fluoro-2-methoxyphenyl) acetate as a yellow oil (500 mg, 87%). LCMS (ES, m/z): 256 [M+H]⁺.

### Step 2. 3-(dimethylamino)-2-(5-fluoro-2-methoxyphenyl)propanoic Acid

To a solution of methyl 3-(dimethylamino)-2-(5-fluoro-2-methoxyphenyl)propanoate (500 mg, 1.67 mmol) in THF (5 mL) was added lithium hydroxide (210 mg, 8.32 mmol) and water (5 mL). The mixture was stirred for 2 h at rt. The resulting solution was washed with diethyl ether (1×10 mL) and acidified to pH=5 with hydrochloric acid (0.5 N). The resulting solution was extracted with ethyl acetate (3×10 mL). The combined organic layers were dried over anhydrous sodium sulfate, filtered, and concentrated under vacuum to afford 3-(dimethylamino)-2-(5-fluoro-2-methoxyphenyl)propanoic acid as a white solid (400 mg, 85%). LCMS (ES, m/z): 242 [M+H]⁺.

### Step 3. (2R)-1-[5-(2,3-dihydro-1,4-benzodioxine-6-sulfonyl)-1H,2H,3H,4H,5H,6H-pyrrolo[3,4-c]pyrrol-2-yl]-3-(dimethylamino)-2-(5-fluoro-2-methoxyphenyl)propan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-3-(dimethylamino)-2-(5-fluoro-2-methoxyphenyl)propan-1-one

To a solution of 3-(dimethylamino)-2-(5-fluoro-2-methoxyphenyl)propanoic acid (150 mg, 0.56 mmol) in DMF (3 mL) was added HATU (269 mg, 0.67 mmol), 2-(2,3-dihydro-1,4-benzodioxine-6-sulfonyl)-1H,2H,3H,4H,5H,6H-pyrrolo[3,4-c]pyrrole (218 mg, 0.67 mmol), and DIEA (2.92 mL, 1.68 mmol). The resulting mixture was stirred for 2 h at rt. The reaction was poured into water (5 mL) and then extracted with ethyl acetate (3×5 mL). The combined organic layers were dried over anhydrous sodium sulfate, filtered, and concentrated under vacuum. The resulting crude product was purified by silica gel chromatography (eluting with 3:1 ethyl acetate/petroleum ether) and further purified by Prep-HPLC (Column: XBridge Shield RP18 OBD Column, 5 μm, 19×150 mm; Mobile Phase, A: water (containing 10 mmol/L NH₄HCO₃) and B: MeCN (30% to 55% over 7 min); Flow rate: 20 mL/min; Detector: UV 254 nm). The product fractions were concentrated under vacuum. The two enantiomers were further separated by Chiral Prep-HPLC (Column: CHIRAL ART Cellulose-SB, 5 μm, 20×250 mm; Mobile Phase, A: hexane/DCM (3:1) and B: EtOH (keep 50% B over 10 min); Flow rate: 20 mL/min; Detector: UV 254/220 nm; Retention time: 1^{st}, 6.201 min; 2^{nd}, 8.07 min). The product fractions were concentrated and lyophilized to afford 1-[5-(2,3-dihydro-1,4-benzodioxine-6-sulfonyl)-1H,2H,3H,4H,5H,6H-pyrrolo[3,4-c]pyrrol-2-yl]-3-(dimethylamino)-2-(5-fluoro-2-methoxyphenyl)propan-1-one, 1^{st} eluting isomer, as a white solid (12.1 mg, 3.9%), and 1-[5-(2,3-dihydro-1,4-benzodioxine-6-sulfonyl)-1H,2H,3H,4H,5H,6H-pyrrolo[3,4-c]pyrrol-2-yl]-3-(dimethylamino)-2-(5-fluoro-2-methoxyphenyl)propan-1-one, 2^{nd} eluting isomer, as a white solid (15.3 mg, 4.9%).

### Method I

### Example 102-1. 1-[(1S)-2-[5-(2,3-dihydro-1,4-benzodioxine-6-sulfonyl)-1H,2H,3H,4H,5H,6H-pyrrolo[3,4-c]pyrrol-2-yl]-2-oxo-1-phenylethyl]-3-methylurea

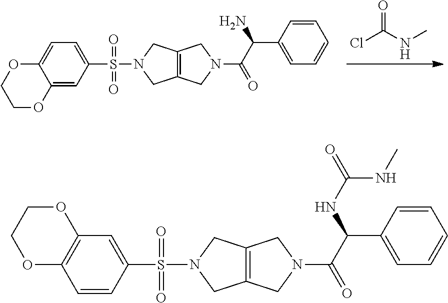

To a solution of (2S)-2-amino-1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one (65 mg, 0.15 mmol) in dichloromethane (5 mL) was added triethylamine (45 mg, 0.44 mmol) and N-methylcarbamoyl chloride (18 mg, 0.19 mmol) at 0° C. The resulting solution was stirred for 3 h at room temperature. The resulting mixture was concentrated under vacuum. The resulting crude product was purified by Prep-HPLC (Column: SunFire Prep C18 column, 5 μm, 19×150 mm; Mobile Phase, A: water (containing 0.1% formic acid) and B: MeCN (15% to 70% over 12 min); Flow rate: 20 mL/min; Detector: UV 254 nm). The product fractions were concentrated under vacuum to afford 1-[(1S)-2-[5-(2,3-dihydro-1,4-benzodioxine-6-sulfonyl)-1H,2H,3H,4H,5H,6H-pyrrolo[3,4-c]pyrrol-2-yl]-2-oxo-1-phenylethyl]-3-methylurea as a white solid (11.1 mg, 15%). ¹H-NMR (CD₃OD, 300 MHz) δ (ppm): 7.41-7.31 (m, 7H), 7.01 (d, J=9.3 Hz, 1H), 5.46 (s, 1H), 4.49-4.44 (m, 1H), 4.33-4.30 (m, 4H), 4.25-4.19 (m, 1H), 4.10-4.04 (m, 5H), 3.92-3.88 (m, 1H), 2.69 (s, 3H). LCMS (ESI, m/z): 499 [M+H]⁺.

### Method J

### (2R)-1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one

### Example 103-1. 1^{st} Eluting Isomer; Example 103-2. 2^{nd} Eluting Isomer

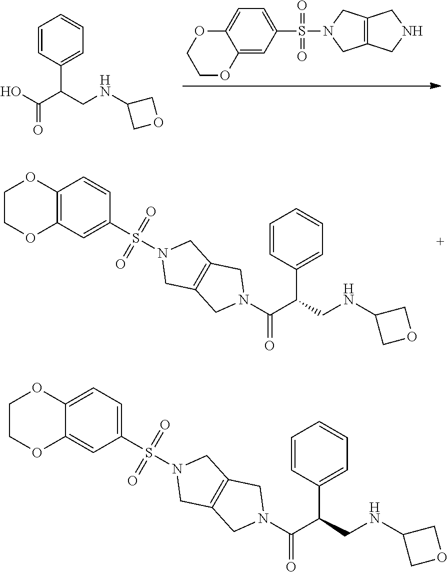 [SMILES: O=C(O)C(CNC1COC1)c1ccccc1 ; O=S(=O)(c1ccc2c(c1)OCCO2)N1CC2=C(CNC2)C1 ; O=C([C@@H](CNC1COC1)c1ccccc1)N1CC2=C(C1)CN(S(=O)(=O)c1ccc3c(c1)OCCO3)C2 ; O=C([C@H](CNC1COC1)c1ccccc1)N1CC2=C(C1)CN(S(=O)(=O)c1ccc3c(c1)OCCO3)C2]

To a solution of 3-[(oxetan-3-yl)amino]-2-phenylpropanoic acid (60 mg, 0.27 mmol) in DMF (10 mL) was added HATU (123 mg, 0.32 mmol), 2-(2,3-dihydro-1,4-benzodioxine-6-sulfonyl)-1H,2H,3H,4H,5H,6H-pyrrolo[3,4-c]pyrrole hydrochloride (93 mg, 0.27 mmol) and DIEA (0.13 mL, 0.81 mmol). The resulting mixture was stirred for 1 h at room temperature. The reaction mixture was poured into water (5 mL) and then extracted with ethyl acetate (3×10 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 30×150 mm; Mobile Phase, A: water (containing 10 mmol/L NH₄HCO₃) and B: CH₃CN (25% to 45% over 7 min); Flow rate: 60 mL/min; Detector: UV 254 nm). The product fractions were concentrated under vacuum. The two enantiomers were further separated by Chiral Prep-HPLC (Column: CHIRALPAK IF, 5 μm, 20×250 mm; Mobile Phase, A: MeOH (containing 0.1% DEA) and B: DCM (keep 10% B over 16 min); Flow rate: 20 mL/min; Detector: UV 254/220 nm; Retention time: 1^{st}, 17.285 min; 2^{nd}, 21.532 min). The product fractions were concentrated and lyophilized to afford 1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one, 1^{st} eluting isomer, as a white solid (1 mg, 1.4%), and 1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one, 2^{nd} eluting isomer, as a white solid (1 mg, 1.4%).

### Method K

### (2R)-1-[5-(2,3-dihydro-1,4-benzodioxine-6-sulfonyl)-1H,2H,3H,4H,5H,6H-pyrrolo[3,4-c]pyrrol-2-yl]-2-[[1-(2-hydroxyethyl)azetidin-3-yl]oxy]-2-phenylethan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-2-[[1-(2-hydroxyethyl)azetidin-3-yl]oxy]-2-phenylethan-1-one

### Example 104-1. 1^{st} Eluting Isomer: Example 104-2. 2^{nd} Eluting Isomer

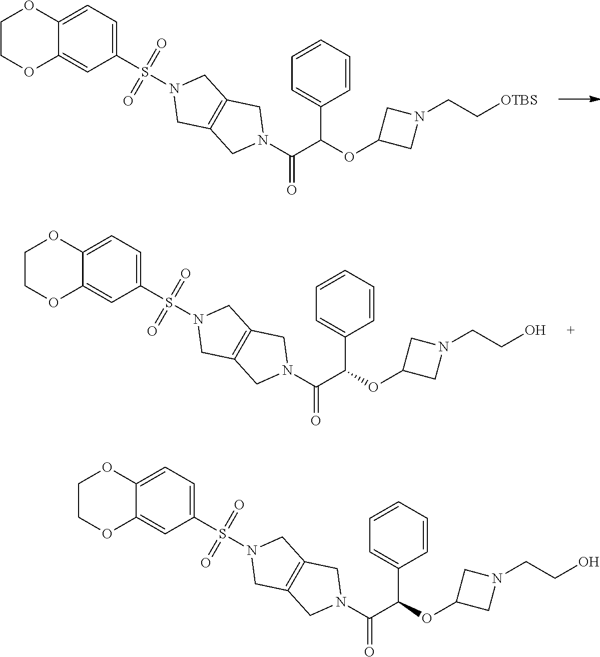 [SMILES: CC(C)(C)[Si](C)(C)OCCN1CC(OC(C(=O)N2CC3=C(C2)CN(S(=O)(=O)c2ccc4c(c2)OCCO4)C3)c2ccccc2)C1 ; O=C([C@@H](OC1CN(CCO)C1)c1ccccc1)N1CC2=C(C1)CN(S(=O)(=O)c1ccc3c(c1)OCCO3)C2 ; O=C([C@H](OC1CN(CCO)C1)c1ccccc1)N1CC2=C(C1)CN(S(=O)(=O)c1ccc3c(c1)OCCO3)C2]

To a solution of 2-[(1-[2-[(tert-butyldimethylsilyl)oxy]ethyl]azetidin-3-yl)oxy]-1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one (150 mg, 0.20 mmol) in acetonitrile (1.9 mL) was added hydrofluoric acid (0.1 mL). The resulting mixture was stirred for 18 h at 25° C. The reaction mixture was concentrated and dissolved in methanol (2 mL). The resulting solution was basified to pH=8 with ammonia (7 M in MeOH) and then concentrated under vacuum. The crude product was purified by Prep-HPLC (Column: XBridge Prep C18 OBD Column, 5 μm, 19×150 mm; Mobile Phase, A: water (containing 10 mmol/L NH₄HCO₃) and B: MeCN (10% to 50% over 8 min); Flow rate: 20 mL/min; Detector: UV 254 nm). The product fractions were concentrated under vacuum. The two enantiomers were separated by Chiral Prep-HPLC (Column: CHIRAL ART Cellulose-SB, 5 μm, 20×250 mm; Mobile Phase, A: MeOH (containing 0.1% IPA) and B: DCM (keep 100% A over 13 min); Flow rate: 14 mL/min; Detector: UV 254/220 nm; Retention time: 1^{st}, 7.566 min; 2^{nd}, 8.977 min). The product fractions were concentrated and lyophilized to 1-[5-(2,3-dihydro-1,4-benzodioxine-6-sulfonyl)-1H,2H,3H,4H,5H,6H-pyrrolo[3,4-c]pyrrol-2-yl]-2-[[1-(2-hydroxyethyl)azetidin-3-yl]oxy]-2-phenylethan-1-one, 1^{st} eluting isomer, as a light yellow solid (3.2 mg, 6%), and 1-[5-(2,3-dihydro-1,4-benzodioxine-6-sulfonyl)-1H,2H,3H,4H,5H,6H-pyrrolo[3,4-c]pyrrol-2-yl]-2-[[1-(2-hydroxyethyl)azetidin-3-yl]oxy]-2-phenylethan-1-one, 2^{nd} eluting isomer, as a light yellow solid (1.8 mg, 3%).

### Method L

### (2S)-1-[5-(2,3-dihydro-1,4-benzodioxine-6-sulfonyl)-1H,2H,3H,4H,5H,6H-pyrrolo[3,4-c]pyrrol-2-yl]-3-(3-fluoroazetidin-3-yl)-2-phenylpropan-1-one and (2R)-1-[5-(2,3-dihydro-1,4-benzodioxine-6-sulfonyl)-1H,2H,3H,4H,5H,6H-pyrrolo[3,4-c]pyrrol-2-yl]-3-(3-fluoroazetidin-3-yl)-2-phenylpropan-1-one

### Example 105-1. 1^{st} Eluting Isomer; Example 105-2. 2^{nd} Eluting Isomer

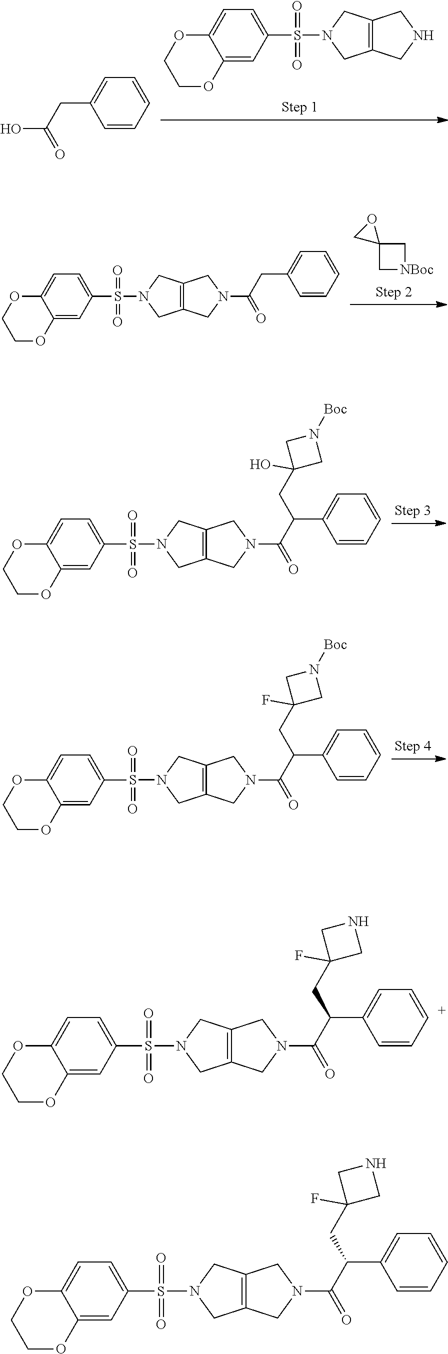

### Step 1. 1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one

To a solution of 2-phenylacetic acid (79 mg, 0.58 mmol) in N,N-dimethylformamide (2 mL) was added 2-(2,3-dihydro-1,4-benzodioxine-6-sulfonyl)-1H,2H,3H,4H,5H,6H-pyrrolo[3,4-c]pyrrole hydrochloride (200 mg, 0.58 mmol), DIEA (0.17 mL, 1.05 mmol) and HATU (331 mg, 0.87 mmol). The resulting mixture was stirred for 2 h at room temperature. The reaction mixture was poured into brine (2 mL) and then extracted with EA (3×2 mL). The combined organic layers were dried over anhydrous sodium sulfate, filtered and concentrated under vacuum. The resulting crude product was purified by silica gel chromatography (eluting with 1:20 EA/PE) to afford 1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one as a light brown solid (150 mg, 61%). LCMS (ES, m/z): 427 [M+H]⁺.

### Step 2. Tert-butyl 3-[3-[5-(2,3-dihydro-1,4-benzodioxine-6-sulfonyl)-1H,2H,3H,4H,5H,6H-pyrrolo[3,4-c]pyrrol-2-yl]-3-oxo-2-phenylpropyl]-3-hydroxyazetidine-1-carboxylate

To a solution of 1-[5-(2,3-dihydro-1,4-benzodioxine-6-sulfonyl)-1H,2H,3H,4H,5H,6H-pyrrolo[3,4-c]pyrrol-2-yl]-2-phenylethan-1-one (150 mg, 0.35 mmol) in tetrahydrofuran (2 mL) in 8 mL vial was added lithium hexamethyldisilazide (0.5 mL, 1 M in THF) at −60° C. The resulting mixture was stirred for 45 min at −60° C. The reaction was treated with tert-butyl 1-oxa-5-azaspiro[2.3]hexane-5-carboxylate (66 mg, 0.36 mmol). The resulting mixture was allowed to warm to room temperature and stirred overnight. The reaction mixture was poured into brine (2 mL) and then extracted with EtOAc (3×2 mL). The combined organic layers were dried over anhydrous sodium sulfate, filtered and concentrated under vacuum. The resulting crude product was purified by silica gel chromatography (eluting with 1:3 EtOAc/petroleum ether) to afford tert-butyl 3-[3-[5-(2,3-dihydro-1,4-benzodioxine-6-sulfonyl)-1H,2H,3H,4H,5H,6H-pyrrolo[3,4-c]pyrrol-2-yl]-3-oxo-2-phenylpropyl]-3-hydroxyazetidine-1-carboxylate as a light yellow oil (110 mg, 51%). LCMS (ES, m/z): 612 [M+H]⁺.

### Step 3. Tert-butyl 3-[3-[5-(2,3-dihydro-1,4-benzodioxine-6-sulfonyl)-1H,2H,3H,4H,5H,6H-pyrrolo[3,4-c]pyrrol-2-yl]-3-oxo-2-phenylpropyl]-3-fluoroazetidine-1-carboxylate

To a solution of tert-butyl 3-[3-[5-(2,3-dihydro-1,4-benzodioxine-6-sulfonyl)-1H,2H,3H,4H,5H,6H-pyrrolo[3,4-c]pyrrol-2-yl]-3-oxo-2-phenylpropyl]-3-hydroxyazetidine-1-carboxylate (110 mg, 0.18 mmol) in dichloromethane (1 mL) was added DAST (29 mg, 0.18 mmol) at 0° C. The resulting mixture was stirred overnight at room temperature. The reaction mixture was poured into brine (2 mL) and then extracted with DCM (3×2 mL). The combined organic layers were dried over anhydrous sodium sulfate, filtered and concentrated under vacuum. The resulting crude product was purified by silica gel chromatography (eluting with 1:3 EtOAc/petroleum ether) to afford tert-butyl 3-[3-[5-(2,3-dihydro-1,4-benzodioxine-6-sulfonyl)-1H,2H,3H,4H,5H,6H-pyrrolo[3,4-c]pyrrol-2-yl]-3-oxo-2-phenylpropyl]-3-fluoroazetidine-1-carboxylate as a light yellow oil (40 mg, 36%). LCMS (ES, m/z): 614 [M+H]⁺.

### Step 4. (2S)-1-[5-(2,3-dihydro-1,4-benzodioxine-6-sulfonyl)-1H,2H,3H,4H,5H,6H-pyrrolo[3,4-c]pyrrol-2-yl]-3-(3-fluoroazetidin-3-yl)-2-phenylpropan-1-one and (2R)-1-[5-(2,3-dihydro-1,4-benzodioxine-6-sulfonyl)-1H,2H,3H,4H,5H,6H-pyrrolo[3,4-c]pyrrol-2-yl]-3-(3-fluoroazetidin-3-yl)-2-phenylpropan-1-one

To a solution of tert-butyl 3-[3-[5-(2,3-dihydro-1,4-benzodioxine-6-sulfonyl)-1H,2H,3H,4H,5H,6H-pyrrolo[3,4-c]pyrrol-2-yl]-3-oxo-2-phenylpropyl]-3-fluoroazetidine-1-carboxylate (35 mg, 0.06 mmol) in dichloromethane (1 mL) was added trifluoroacetic acid (0.2 mL) at 0° C. The resulting mixture was stirred for 1 h at room temperature. The pH value of the solution was adjusted to 9 with ammonia (7 M in MeOH) and then concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: MeCN (5% to 40% over 7 min); Flow rate: 20 mL/min; Detector: UV 254 nm). The product fractions were concentrated under vacuum. The two enantiomers were separated by Chiral Prep-HPLC (Column: CHIRALPAK IC, 5 μm, 20×250 mm; Mobile Phase, A: MeOH (containing 0.1% DEA) and B: DCM (keep 50% B over 15 min); Flow rate: 18 mL/min; Detector: UV 254/220 nm; Retention time: 1^{st}, 7.87 min; 2^{nd}, 11.8 min). The product fractions were concentrated and lyophilized to afford 1-[5-(2,3-dihydro-1,4-benzodioxine-6-sulfonyl)-1H,2H,3H,4H,5H,6H-pyrrolo[3,4-c]pyrrol-2-yl]-3-(3-fluoroazetidin-3-yl)-2-phenylpropan-1-one, 1^{st} eluting isomer, as a white solid (2.1 mg, 14%), and 1-[5-(2,3-dihydro-1,4-benzodioxine-6-sulfonyl)-1H,2H,3H,4H,5H,6H-pyrrolo[3,4-c]pyrrol-2-yl]-3-(3-fluoroazetidin-3-yl)-2-phenylpropan-1-one, 2^{nd} eluting isomer, as a white solid (0.4 mg, 3%).

### Method M

### Example 106-1. N-[(1S)-2-[5-(2,3-dihydro-1,4-benzodioxine-6-sulfonyl)-1H,2H,3H,4H,5H,6H-pyrrolo[3,4-c]pyrrol-2-yl]-2-oxo-1-phenylethyl]methanesulfonamide

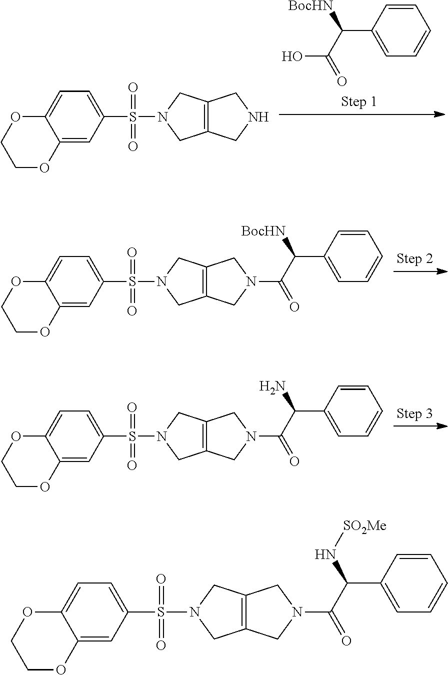 [SMILES: CC(C)(C)OC(=O)N[C@H](C(=O)O)c1ccccc1 ; O=S(=O)(c1ccc2c(c1)OCCO2)N1CC2=C(CNC2)C1 ; CC(C)(C)OC(=O)N[C@H](C(=O)N1CC2=C(C1)CN(S(=O)(=O)c1ccc3c(c1)OCCO3)C2)c1ccccc1 ; N[C@H](C(=O)N1CC2=C(C1)CN(S(=O)(=O)c1ccc3c(c1)OCCO3)C2)c1ccccc1 ; CS(=O)(=O)N[C@H](C(=O)N1CC2=C(C1)CN(S(=O)(=O)c1ccc3c(c1)OCCO3)C2)c1ccccc1]

To a solution of 2-((2,3-dihydrobenzo[b][1,4]dioxin-6-yl)sulfonyl)-1,2,3,4,5,6-hexahydropyrrolo[3,4-c]pyrrole core (1 eq) was added (S)-2-((tert-butoxycarbonyl)amino)-2-phenylacetic acid (1.1 eq), HATU (1.2 eq) and DIEA (6 eq). The reaction was stirred at 25° C. for 3 days and then dried under a stream of nitrogen. The material was taken up in 4N HCl in dioxane (13.3 eq) and stirred for 5 h at 50° C. Upon cooling to 23° C., the solvent was removed with a stream of nitrogen and then diluted with toluene. The solvent was azeotroped with toluene by removing with a stream of nitrogen and then dissolved in DCE. To the solution was added methanesulfonic anhydride (3 eq), DIEA (3 eq), and DMAP as a 0.2M solution in DCE (1eq). The reaction stirred at 80° C. for 18 h and was then diluted with Ethyl Acetate and brine. The organic layer was collected, dried under a stream of nitrogen, and submitted for HPLC purification.

### Method N

### Example 107-1. (2S)-1-[5-(3,4-dihydro-2H-1-benzopyran-6-sulfonyl)-1H,2H,3H,4H,5H,6H-pyrrolo[3,4-c]pyrrol-2-yl]-2-hydroxy-2-[3-(4-methylpiperazin-1-yl)phenyl]ethan-1-one

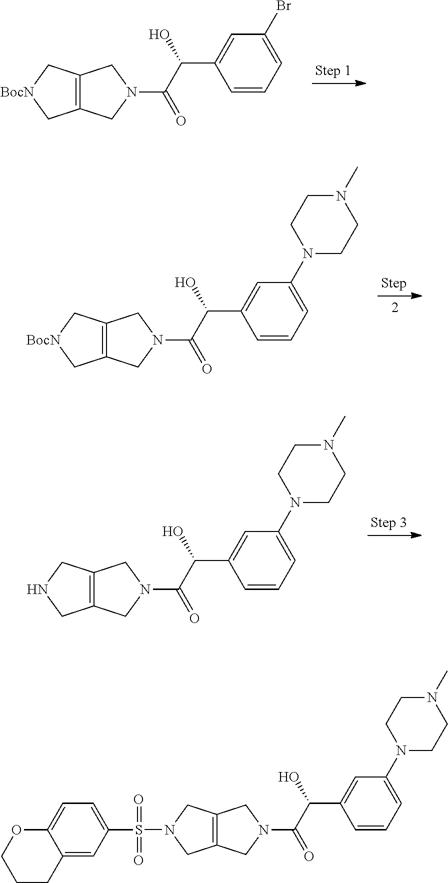 [SMILES: CC(C)(C)OC(=O)N1CC2=C(C1)CN(C(=O)[C@H](O)c1cccc(Br)c1)C2 ; CN1CCN(c2cccc([C@@H](O)C(=O)N3CC4=C(CN(C(=O)OC(C)(C)C)C4)C3)c2)CC1 ; CN1CCN(c2cccc([C@@H](O)C(=O)N3CC4=C(CNC4)C3)c2)CC1 ; CN1CCN(c2cccc([C@@H](O)C(=O)N3CC4=C(C3)CN(S(=O)(=O)c3ccc5c(c3)CCCO5)C4)c2)CC1]

To a solution of 2-((2,3-dihydrobenzo[b][1,4]dioxin-6-yl)sulfonyl)-1,2,3,4,5,6-hexahydropyrrolo[3,4-c]pyrrole (30 umol) and aryl bromide (30 mmol) in dioxane was added Cs₂CO₃ (3 eq), Pd(PPh₃)₄(0.1 eq) and secondary amine added as a 0.2 M solution in dioxane. The reaction was shaken at 100° C. for 18 h. The products were dried under a stream of nitrogen and diluted with dioxane (200 uL) and 4M HCl in dioxane (100 uL). The reaction was shaken at 50° C. for 4 h. The products were dried under a stream of nitrogen and sulfonyl chloride (43 umol) as a 0.2 M solution in dioxane was added. The reaction was shaken at 23° C. for 18 h. The products were dried under a stream of nitrogen and the material was diluted with EtOAc (0.5 mL) and shaken at 50° C. until solid material was dissolved. LLSE w/ DMT SPE cartridges was performed. 0.5 mL of brine was added and the organic layer was extracted to DMT columns. 0.5 mL EtOAc was added to vials and extracted with DMT columns. The columns were washed with 3 mL of 3:1 EtOAc/MeOH. The solutions were dried and dissolved in 0.5 mL DMSO for preparatory reverse phase HPLC.

### Method O

### Example 108-1. (2S)-1-[5-(1-benzofuran-6-sulfonyl)-1H,2H,3H,4H,5H,6H-pyrrolo[3,4-c]pyrrol-2-yl]-2-hydroxy-2-(2-methyl-1,3-benzoxazol-4-yl)ethan-1-one

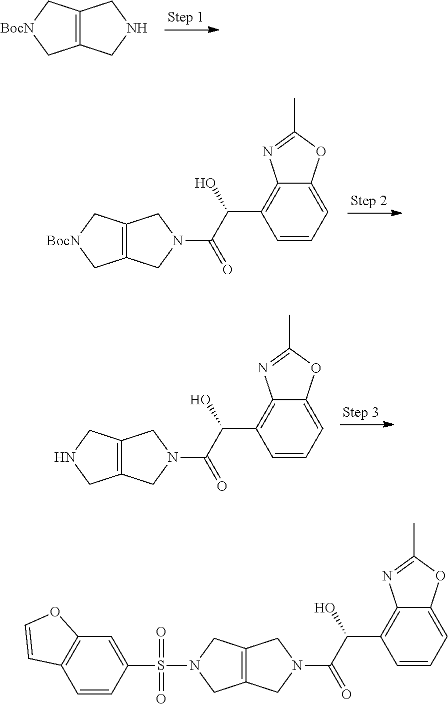 [SMILES: CC(C)(C)OC(=O)N1CC2=C(CNC2)C1 ; Cc1nc2c([C@@H](O)C(=O)N3CC4=C(CN(C(=O)OC(C)(C)C)C4)C3)cccc2o1 ; Cc1nc2c([C@@H](O)C(=O)N3CC4=C(CNC4)C3)cccc2o1 ; Cc1nc2c([C@@H](O)C(=O)N3CC4=C(C3)CN(S(=O)(=O)c3ccc5ccoc5c3)C4)cccc2o1]

To a solution of tert-butyl 3,4,5,6-tetrahydropyrrolo[3,4-c]pyrrole-2(1H)-carboxylate (1 eq) and (R)-2-hydroxy-2-(2-methylbenzo[d]oxazol-4-yl)acetic acid (1.1 eq) was added HATU (1.2 eq), and DIEA (5 eq) at 50° C. for 2 hours. The reaction was concentrated under a stream of nitrogen and diluted with Ethyl Acetate and saturated aqueous sodium bicarbonate solution. The organic layers were collected and dried under vacuum. The material was treated with 4N HCl in dioxane at 50° C. for two hours and then concentrated under a stream of nitrogen. Water was removed by adding toluene and concentrating under a stream of nitrogen. To the material was added benzofuran-6-sulfonyl chloride as a solution in DCE and DIEA (5 eq). After two hours, the solvent was removed under a stream of nitrogen and the residue was diluted with ethyl acetate and aqueous saturated sodium bicarbonate solution. The organic layers were collected, concentrated under a stream of nitrogen and submitted for HPLC purification.

### Method OA

### Example 109-1. 1-(5-((2,3-dihydrobenzo[b][1,4]dioxin-6-yl)sulfonyl)-5,6-dihydropyrrolo[3,4-c]pyrrol-2(1H,3H,4H)-yl)-2-hydroxy-2-(3-morpholinophenyl)ethanone

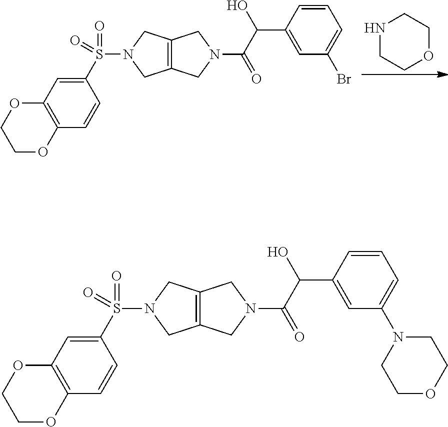

To a solution of 2-(3-bromophenyl)-1-(5-((2,3-dihydrobenzo[b][1,4]dioxin-6-yl)sulfonyl)-5,6-dihydropyrrolo[3,4-c]pyrrol-2(1H,3H,4H)-yl)-2-hydroxyethanone (0.2 M anhydrous 1,4-dioxane, 100 μL, 20 μmol) was added morpholine (0.2 M anhydrous 1,4-dioxane, 150 μL, 30 μmol), XPhos Pd-G2 precatalyst (0.02 M anhydrous 1,4-dioxane, 50 μL, 1 μmol) and cesium carbonate (26 mg, 0.08 mmol). The resulting mixture was heated under nitrogen atmosphere at 100° C. overnight. After cooling to room temperature, the mixture was diluted with brine (0.35 mL) and EtOAc (0.5 mL). The organic layer was separated and the aqueous layer was extracted again with EtOAc (0.6 mL). The combined organic layers were concentrated in vacuo, and the residue was purified by HPLC. RuPhos 3G can also be used as catalyst in arylhalide/amine crosscoupling reactions.

### Method P

### (2S)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-methoxy-2-(2-methyl-1,3-benzoxazol-4-yl)ethan-1-one and (2R)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-methoxy-2-(2-methyl-1,3-benzoxazol-4-yl)ethan-1-one

### Example 110-1. 1^{st} Eluting Isomer; Example 110-2. 2^{nd} Eluting Isomer

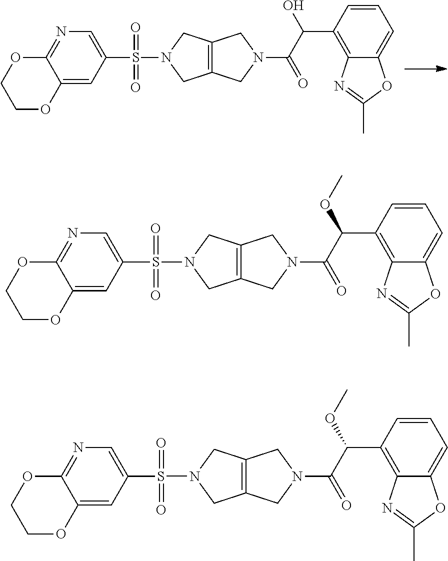 [SMILES: Cc1nc2c(C(O)C(=O)N3CC4=C(C3)CN(S(=O)(=O)c3cnc5c(c3)OCCO5)C4)cccc2o1 ; CO[C@H](C(=O)N1CC2=C(C1)CN(S(=O)(=O)c1cnc3c(c1)OCCO3)C2)c1cccc2oc(C)nc12 ; CO[C@@H](C(=O)N1CC2=C(C1)CN(S(=O)(=O)c1cnc3c(c1)OCCO3)C2)c1cccc2oc(C)nc12]

To a solution of 1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one (100 mg, 0.20 mmol) in dichloromethane (5 mL), was added iodomethane (316 mg, 2.23 mmol) and silver oxide (140 mg, 0.60 mmol). The resulting mixture was stirred for 24 h at room temperature. The reaction mixture was filtered and concentrated under vacuum. The enantiomers were separated by Chiral Prep-HPLC (Column: CHIRAL IC, 2×25 cm, 5 μm; Mobile Phase A: DCM, Mobile Phase B: MeOH; Flow rate: 17 mL/min; Gradient: 50 B to 50 B over 17 min; Detector: UV 254/220 nm; Retention time: 1^{st}, 12.78 min; 2^{nd}, 15.03 min). The product fractions were concentrated and lyophilized to afford 1-(5-[2H,3H-[1,4]dioxino[2,3-b]pyridine-7-sulfonyl]-1H,2H,3H,4H,5H,6H-pyrrolo [3,4-c]pyrrol-2-yl)-2-methoxy-2-(2-methyl-1,3-benzoxazol-4-yl)ethan-1-one, 1^{st} eluting isomer, as a white solid (6.3 mg, 6%), and 1-(5-[2H,3H-[1,4]dioxino[2,3-b]pyridine-7-sulfonyl]-1H,2H,3H,4H,5H,6H-pyrrolo[3,4-c]pyrrol-2-yl)-2-methoxy-2-(2-methyl-1,3-benzoxazol-4-yl)ethan-1-one, 2^{nd} eluting isomer, as a white solid (8.0 mg, 8%).

### Method Q

### (2S)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxy-2-(3-{2-oxa-7-azaspiro[3.5]nonan-7-yl}phenyl)ethan-1-one and (2R)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxy-2-(3-{2-oxa-7-azaspiro[3.5]nonan-7-yl}phenyl)ethan-1-one

### Example 111-1. 1^{st} Eluting Isomer; Example 111-2. 2^{nd} Eluting Isomer

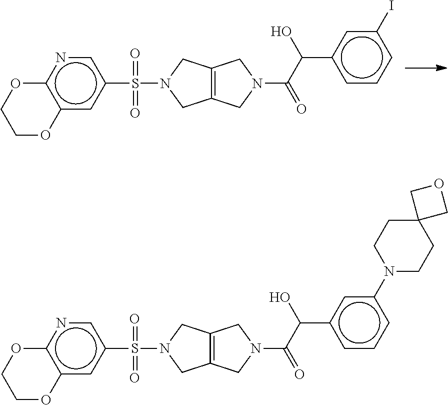

To a solution of 1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(3-iodophenyl)ethan-1-one (75 mg, 0.12 mmol) in 1,4-dioxane (2 mL), was added 2-oxa-7-azaspiro[3.5]nonane (100 mg, 0.72 mmol), RuPhos (12 mg, 0.024 mmol), potassium phosphate (84 mg, 0.36 mmol) and RuPhos 3G (22 mg, 0.024 mmol). The resulting mixture was stirred for 18 h at 100° C. and then cooled to room temperature. The reaction mixture was poured into water (5 mL) and then extracted with ethyl acetate (3×10 mL). The combined organic layers were dried over anhydrous sodium sulfate, filtered, and concentrated under vacuum. The resulting crude product was purified by Prep-HPLC (Column: XBridge Prep C18 OBD Column, 19×150 mm, 5 μm; Mobile Phase A: water (10 mmol/L NH₄HCO₃), Mobile Phase B: MeCN; Gradient: 25% B to 55% B over 7 min; Flow rate: 20 mL/min; Detector: UV 254 nm). The product fractions were concentrated under vacuum. The two enantiomers were separated by Chiral Prep-HPLC (Column: CHIRALPAK IF, 2×25 cm, 5 μm; Mobile Phase A: DCM, Mobile Phase B: MeOH; Flow rate: 17 mL/min; Gradient: 50 B to 50 B over 12 min; Detector: UV 254/220 nm; Retention time: 1^{st}, 8.01 min; 2^{nd}, 10.07 min). The product fractions were concentrated and lyophilized to afford 1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(3-[2-oxa-7-azaspiro[3.5]nonan-7-yl]phenyl)ethan-1-one, 1^{st} eluting isomer, as a white solid (6.2 mg, 9%), and 1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(3-[2-oxa-7-azaspiro[3.5]nonan-7-yl]phenyl)ethan-1-one, 2^{nd} eluting isomer, as a white solid (6.2 mg, 9%).

### Method R

### (2S)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one and (2R)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one

### Example 112-1. 1^{st} Eluting Isomer; Example 112-2. 2^{nd} Eluting Isomer

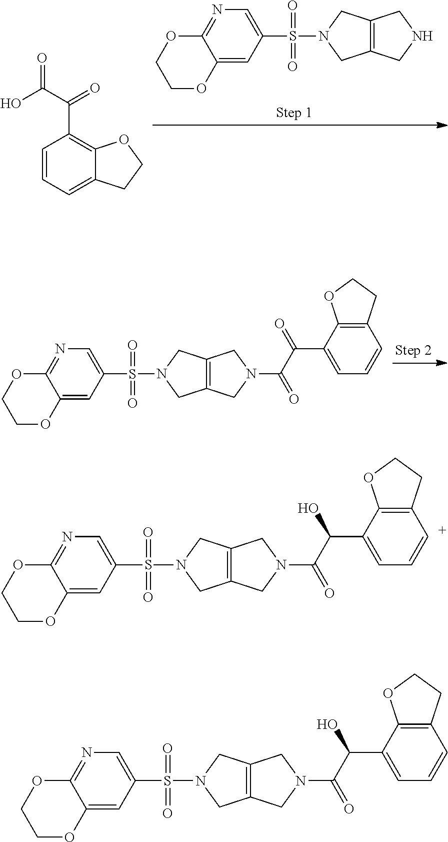 [SMILES: O=C(O)C(=O)c1cccc2c1OCC2 ; O=S(=O)(c1cnc2c(c1)OCCO2)N1CC2=C(CNC2)C1 ; O=C(C(=O)N1CC2=C(C1)CN(S(=O)(=O)c1cnc3c(c1)OCCO3)C2)c1cccc2c1OCC2 ; O=C([C@@H](O)C1=CC=[C+]C2=C1OCC2)N1CC2=C(C1)CN(S(=O)(=O)c1cnc3c(c1)OCCO3)C2 ; O=C([C@@H](O)c1cccc2c1OCC2)N1CC2=C(C1)CN(S(=O)(=O)c1cnc3c(c1)OCCO3)C2]

### Step 1. 1-(2,3-dihydro-1-benzofuran-7-yl)-2-(5-{2H,3H-[1,4]dioxino[2,3-b]pyridine-7-sulfonyl}-1H,2H,3H,4H,5H,6H-pyrrolo[3,4-c]pyrrol-2-yl)ethane-1,2-dione

To a solution of 2-(2,3-dihydro-1-benzofuran-7-yl)-2-oxoacetic acid (250 mg, 1.30 mmol) in DMF (4 mL) was added 2-[2H,3H-[1,4]dioxino[2,3-b]pyridine-7-sulfonyl]-1H,2H,3H,4H,5H,6H-pyrrolo[3,4-c]pyrrole (450 mg, 1.30 mmol), DIEA (0.43 mL, 2.60 mmol), and HATU (544 mg, 1.43 mmol). The resulting solution was stirred for 1 h at room temperature. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by prep-TLC (eluting with 1:1 ethyl acetate/petroleum ether) to afford 1-(2,3-dihydro-1-benzofuran-7-yl)-2-(5-{2H,3H-[1,4]dioxino[2,3-b]pyridine-7-sulfonyl}-1H,2H,3H,4H,5H,6H-pyrrolo[3,4-c]pyrrol-2-yl)ethane-1,2-dione as a light yellow solid (200 mg, 32%). LCMS (ES, m/z): 484 [M+H]⁺.

### Step 2. (2S)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one and (2R)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one

To a solution of 1-(2,3-dihydro-1-benzofuran-7-yl)-2-(5-{2H,3H-[1,4]dioxino[2,3-b]pyridine-7-sulfonyl}-1H,2H,3H,4H,5H,6H-pyrrolo[3,4-c]pyrrol-2-yl)ethane-1,2-dione (200 mg, 0.41 mmol) in tetrahydrofuran (2 mL) was added sodium borohydride (8 mg, 0.21 mmol). The resulting solution was stirred for 30 min at 0° C. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: MeCN (15% to 45% over 10 min); Flow rate: 20 mL/min; Detector: UV 254 nm). The product fractions were concentrated under vacuum. The two enantiomers were separated by Chiral Prep-HPLC (Column: CHIRALPAK IF, 5 μm, 20×250 mm; Mobile Phase, A: MeOH (containing 0.1% DEA) and B: DCM (keep 40% B over 50 min); Flow rate: 15 mL/min; Detector: UV 254/220 nm; Retention time: 1^{st}, 19.223 min; 2^{nd}, 29.404 min). The product fractions were concentrated and lyophilized to afford 2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one, 1^{st} eluting isomer, as a white solid (30.5 mg, 15%), and 2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one, 2^{nd} eluting isomer, as a white solid (33.5 mg, 17%).

### Method S

### Example 114-1. (R)-3-((cyclopentylmethyl)amino)-1-(5-((2,3-dihydrobenzo[b][1,4]dioxin-6-yl)sulfonyl)-3,4,5,6-tetrahydropyrrolo[3,4-c]pyrrol-2(1H)-yl)-2-phenylpropan-1-one

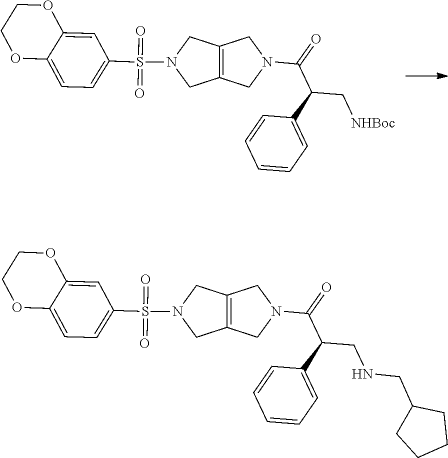 [SMILES: CC(C)(C)OC(=O)NC[C@H](C(=O)N1CC2=C(C1)CN(S(=O)(=O)c1ccc3c(c1)OCCO3)C2)c1ccccc1 ; O=C([C@@H](CNCC1CCCC1)c1ccccc1)N1CC2=C(C1)CN(S(=O)(=O)c1ccc3c(c1)OCCO3)C2]

To tert-butyl (R)-(3-(5-((2,3-dihydrobenzo[b][1,4]dioxin-6-yl)sulfonyl)-3,4,5,6-tetrahydropyrrolo[3,4-c]pyrrol-2(1H)-yl)-3-oxo-2-phenylpropyl)carbamate (180 μl, 36.0 μmol; 0.2M in dioxane) was added acetic acid (150 μl, 30.0 μmol; 0.2M in dioxane) and DCE, cyclopentanecarbaldehyde (180 μl, 36.0 μmol; 0.2M in dioxane) and sodium triacetoxyborohydride (300 μl, 60.0 μmol; 0.2M in dioxane). The reaction was heated at 50° C. for 4 h. The reaction was run through an SCX-SPE cartridge and eluted with 2 ml of 10% MeOH/EtOAc (ETW) followed by 2 ml of 2M Ammonia/MeOH (ETC). The basic eluent was dried under a stream of N₂ and the product was purified by reverse phase HPLC.

### Method T

### Example 115. (2S)-1-(5-(2,3-Dihydrobenzo[b][1,4]dioxine-6-sulfonimidoyl)-3,4,5,6-tetrahydropyrrolo[3,4-c]pyrrol-2(1H)-yl)-2-hydroxy-2-phenylethan-1-one

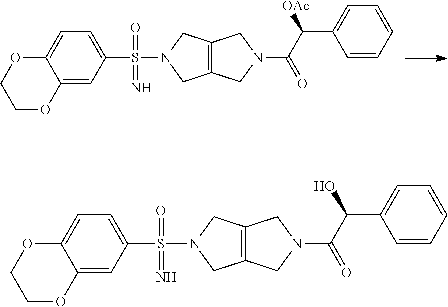 [SMILES: CC(=O)O[C@H](C(=O)N1CC2=C(C1)CN(S(=N)(=O)c1ccc3c(c1)OCCO3)C2)c1ccccc1 ; N=S(=O)(c1ccc2c(c1)OCCO2)N1CC2=C(CN(C(=O)[C@@H](O)c3ccccc3)C2)C1]

A mixture of impure (1S)-2-(5-(2,3-dihydrobenzo[b][1,4]dioxine-6-sulfonimidoyl)-3,4,5,6-tetrahydropyrrolo[3,4-c]pyrrol-2(1H)-yl)-2-oxo-1-phenylethyl acetate (100.5 mg, 0.208 mmol) (about half the material is already deacetylated) and potassium carbonate (35.0 mg, 0.253 mmol) was treated with MeOH (6 ml) and stirred at ambient temperature 70 minutes. The solution was diluted with EtOAc (75 mL) and washed sequentially with water (75 mL) and brine (25 mL). The organic layer was dried (Na₂SO₄), filtered, treated with silica gel, and evaporated under reduced pressure. The material was chromatographed by Biotage MPLC (10 g silica gel column, 0 to 4% MeOH in DCM) to provide (2S)-1-(5-(2,3-dihydrobenzo[b][1,4]dioxine-6-sulfonimidoyl)-3,4,5,6-tetrahydropyrrolo[3,4-c]pyrrol-2(1H)-yl)-2-hydroxy-2-phenylethan-1-one (31.9 mg, 0.072 mmol, 34.8% yield).

### Method U

### (2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one and (2R)-1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one]

### Example 94-27. 1^{st} Eluting Isomer; Example 94-28. 2^{nd} Eluting Isomer

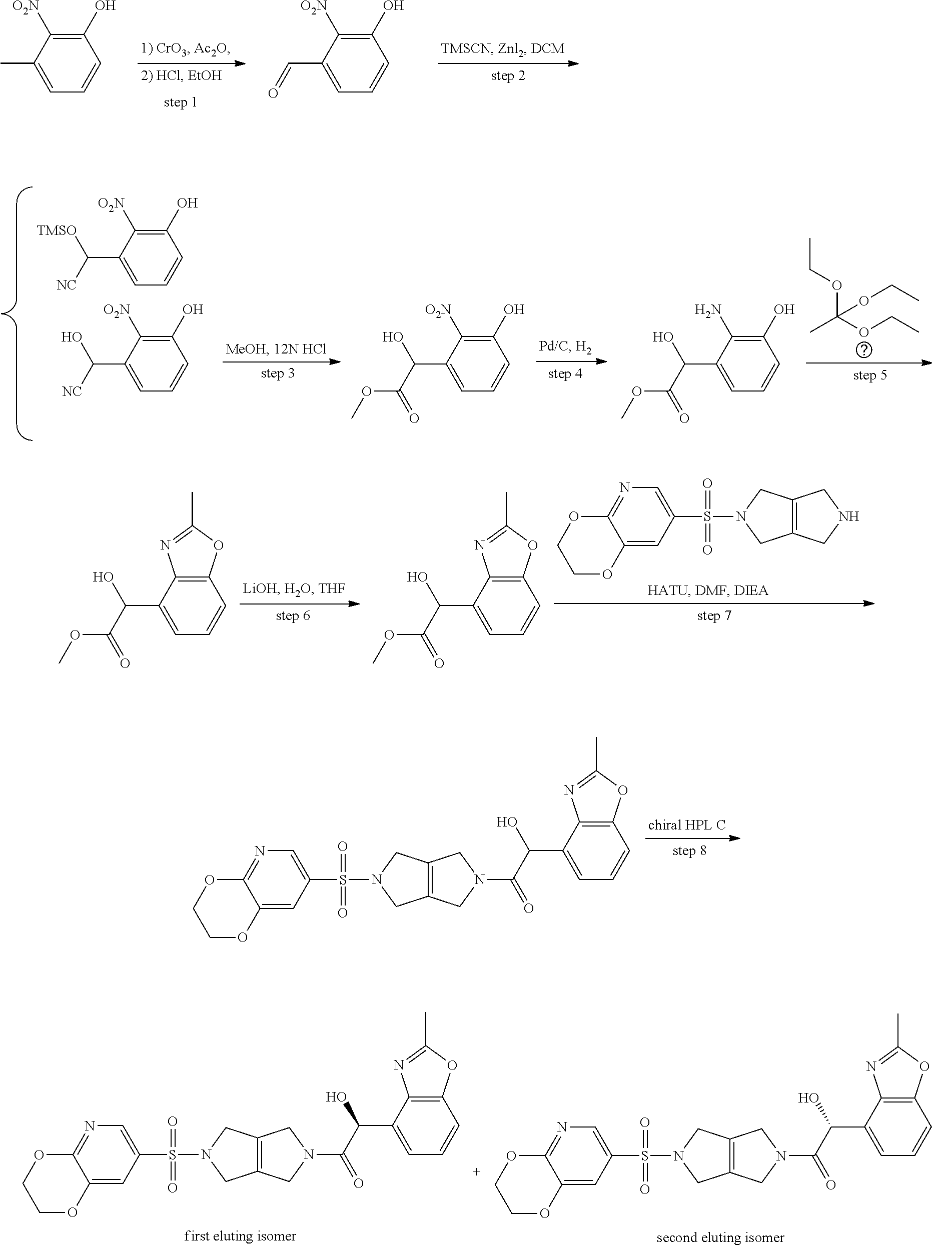

### Step 1. 3-hydroxy-2-nitrobenzaldehyde

Into a 2000-mL 3-necked round-bottom flask, was placed 3-methyl-2-nitrophenol (50 g, 326.51 mmol, 1.00 equiv), HOAc (405 mL), Ac₂O (400 mL), H₂SO₄ (60 mL). Then Cr₂O₃(70 g, 460.56 mmol, 2.14 equiv) was added at 5-10° C. After the addition was completed, stirring was continued for 6 h at the temperature and the dark green mixture was then poured into ice-water. Stirring was continued for 1 h then the mixture was placed in refrigeratory overnight whereupon the reaction product which had solidified was collected and washed with cold water until colorless. The solids were then digested and stirred mechanically for 1.5 h with 360 mL of cold 2% Na₂CO₃ solution. The orange mixture was filtered and the solid washed with water. After drying there was obtained 27 g of colorless material which was hydrolyzed by refluxing for 1.5 h with EtOH (60 mL), H₂O (16 mL), con. HCl (98 mL). The mixture was concentrated under vacuum to give 6.8 g (10%) of 3-hydroxy-2-nitrobenzaldehyde as a brown solid. GCMS (EI, m/z): 167.

### Step 2. 2-hydroxy-2-(3-hydroxy-2-nitrophenyl) acetonitrile

Into a 500-mL 3-necked round-bottom flask, was placed 3-hydroxy-2-nitrobenzaldehyde (4.4 g, 26.33 mmol, 1.00 equiv), DCM (200 mL). This was followed by the addition of TMSCN (7.8 g, 78.62 mmol, 3.00 equiv) and ZnI₂ (829 mg, 2.60 mmol, 0.10 equiv) at 0° C. The resulting solution was stirred for 3 h at room temperature (15° C.). The reaction was then quenched by the addition of 200 mL of brine. The resulting solution was extracted with 3×300 mL of DCM, dried over anhydrous Na₂SO₄, filtered and concentrated under vacuum. The residue was applied onto a silica gel column with EA/PE (1:1) to afford 1.8 g (35%) of 2-hydroxy-2-(3-hydroxy-2-nitrophenyl)acetonitrile as a yellow solid. MS (ESI, m/z): 195[M+H]⁺.

### Step 3. Methyl 2-hydroxy-2-(3-hydroxy-2-nitrophenyl)acetate

Into a 50-mL round-bottom flask, was placed 2-hydroxy-2-(3-hydroxy-2-nitrophenyl) acetonitrile (1.8 g, 9.27 mmol, 1.00 equiv), MeOH (5 ml). This was followed by the addition of HCl (12 moL/L, 5.7 mL, 68.4 mmol, 7.40 equiv). The resulting solution was stirred for 45 min at 60° C. After cooled to room temperature, the resulting mixture was concentrated under vacuum to afford 1.8 g (85%) of methyl 2-hydroxy-2-(3-hydroxy-2-nitrophenyl)acetate as a yellow solid. MS (ESI, m/z): 228[M+H]⁺.

### Step 4. Methyl 2-(2-amino-3-hydroxyphenyl)-2-hydroxyacetate

Into a 100-mL round-bottom flask, was placed methyl 2-hydroxy-2-(3-hydroxy-2-nitrophenyl)acetate (1.8 g, 7.92 mmol, 1.00 equiv), MeOH (30 mL), Pd/C (180 mg). To the above H₂ (g) was introduced in. The resulting mixture was stirred for 4 h at room temperature (13° C.). The solids were filtered out. The resulting mixture was concentrated under vacuum to give 2.0 g (89%, 70% purity) of methyl 2-(2-amino-3-hydroxyphenyl)-2-hydroxyacetate as red oil. The product was used in the next step directly without further purification. MS (ESI, m/z): 198[M+H]⁺.

### Step 5. Methyl 2-hydroxy-2-(2-methyl-1,3-benzoxazol-4-yl)acetate

Into a 20-mL microwave tube, was placed methyl 2-(2-amino-3-hydroxyphenyl)-2-hydroxyacetate (300 mg, 1.52 mmol, 1.00 equiv), Bi(OTf)₃(27 mg, 0.04 mmol, 0.027 equiv), 1,1,1-triethoxyethane (5 mL). The final reaction mixture was irradiated with microwave radiation for 10 min at 85° C. (The reaction was repeated 2 times). After cooled to r.t., the resulting mixture was concentrated under vacuum and purified by silica gel chromatography with PE/EA (0-100%) to give 320 mg (49%) of methyl 2-hydroxy-2-(2-methyl-1,3-benzoxazol-4-yl)acetate as a light yellow solid. MS (ESI, m/z): 222 [M+H]⁺.

### Step 6. Lithium 2-hydroxy-2-(2-methylbenzo[d]oxazol-4-yl)acetate

Into a 50-mL round-bottom flask, was placed methyl 2-hydroxy-2-(2-methyl-1,3-benzoxazol-4-yl)acetate (320 mg, 1.45 mmol, 1.00 equiv), THF (5 mL), H₂O (2 mL), LiOH (174 mg, 7.27 mmol, 5.00 equiv). The resulting solution was stirred for 10 min at room temperature (15° C.). The resulting mixture was concentrated under vacuum, the residue was purified by Flash with the following conditions (IntelFlash-1): Column, C18 silica gel; mobile phase, 10 mmoL/L NH₄HCO₃ in H₂O/ACN=100%/0% increasing to 10 mmoL/L NH₄HCO₃ in H₂O/ACN=70%/30% within 30 min; Detector, UV 220 nm. The collected fraction was concentrated under vacuum to afford 150 mg (crude) of lithium 2-hydroxy-2-(2-methylbenzo[d]oxazol-4-yl)acetate as yellow oil. MS (ESI, m/z): 208 [M+H]⁺.

### Step 7. Synthesis of 1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one

Into a 50-mL round-bottom flask, was placed lithium 2-hydroxy-2-(2-methylbenzo[d]oxazol-4-yl)acetate (67 mg, crude), 2-2H,3H-[1,4]dioxino[2,3-b]pyridine-7-sulfonyl-1H,2H,3H,4H,5H,6H-pyrrolo[3,4-c]pyrrole (100 mg, 0.32 mmol, 1.00 equiv), DMF (10 mL), HATU (148 mg, 0.39 mmol, 1.20 equiv), DIEA (126 mg, 0.974 mmol, 3.00 equiv). The resulting solution was stirred for 1 h at room temperature (18° C.). The mixture was purified by Prep-HPLC with the following conditions (2 #-Analyse HPLC-SHIMADZU (HPLC-10)): Column: XBridge C18 OBD Prep Column, 100A, 5 um, 19 mm×250 mm; Mobile Phase A: H₂O (10 mmoL/L NH₄HCO₃), Mobile Phase B: ACN; Flow rate: 20 mL/min; Gradient: 14% B to 44% B in 8 min; 254 nm. The collected fraction was lyophilized to give 30 mg (19%) of 1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one as a white solid. MS (ESI, m/z): 499 [M+H]⁺.

### Step 8. Chiral Separation of (2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one (First Eluting Isomer) and (2R)-1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one (Second Eluting Isomer) (Absolute Stereochemistry Determined)

1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one (30 mg, 0.06 mmol, 1.00 equiv) was separated by Chiral-Prep-HPLC with the following conditions (Prep-HPLC-009): Column: CHIRALPAK IF, 250×20 mm; Mobile Phase A: DCM-HPLC, Mobile Phase B: MeOH-HPLC; Flow rate: 16 mL/min; Gradient: 60 B to 60 B in 18 min; 254/220 nm. The first eluting isomer (Rt=11.23 min) was collected and concentrated under vacuum then lyophilized to give 5.1 mg (17%) of (2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one as a white solid. The second eluting isomer (Rt=15.39 min) was collected and concentrated under vacuum then lyophilized to give 9.2 mg (31%) of (2R)-1-(5-[2H,3H-[1,4]dioxino[2,3-b]pyridine-7-sulfonyl]-1H,2H,3H,4H,5H,6H-pyrrolo[3,4-c]pyrrol-2-yl)-2-hydroxy-2-(2-methyl-1,3-benzoxazol-4-yl)ethan-1-one as a white solid.

First eluting isomer: ¹H-NMR (400 MHz, DMSO-d6) δ (ppm): 2.63 (s, 3H), 3.88-4.13 (m, 7H), 4.32-4.40 (m, 3H), 4.50-4.52 (m, 2H), 5.70 (s, 2H), 7.32-7.33 (m, 2H), 7.58-7.63 (m, 2H), 8.16 (d, J=2.4 Hz, 1H). MS (ESI, m/z): 499 [M+H]⁺. ee=100%.

Second eluting isomer: ¹H-NMR (400 MHz, DMSO-d6) δ (ppm): 2.63 (s, 3H), 3.88-4.13 (m, 7H), 4.32-4.40 (m, 3H), 4.50-4.52 (m, 2H), 5.70 (s, 2H), 7.31-7.33 (m, 2H), 7.58-7.63 (m, 2H), 8.16 (d, J=2.4 Hz, 1H). MS (ESI, m/z): 499 [M+H]⁺. ee=99.4%.

### Method V

### (2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (First Eluting Isomer) and (2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (Second Eluting Isomer)

### Example 94-31. 1^{st} Eluting Isomer; Example 94-32. 2^{nd} Eluting Isomer

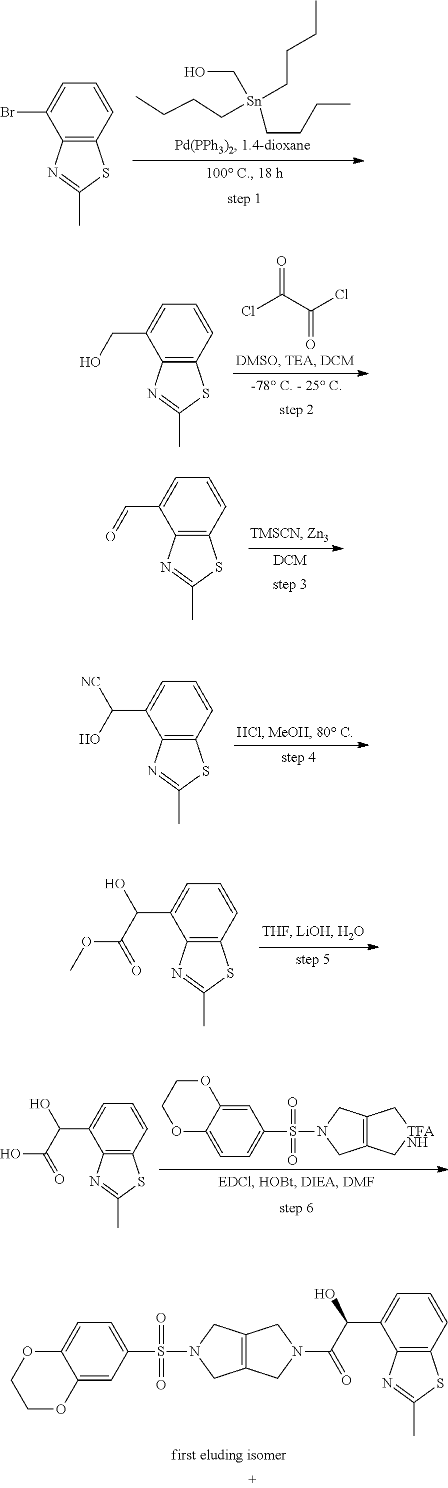

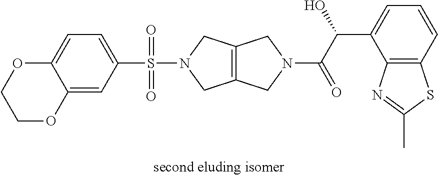 [SMILES: Cc1nc2c([C@@H](O)C(=O)N3CC4=C(C3)CN(S(=O)(=O)c3ccc5c(c3)OCCO5)C4)cccc2s1]

### Step 1. (2-methyl-1,3-benzothiazol-4-yl)methanol

To a mixture of 4-bromo-2-methyl-1,3-benzothiazole (2.1 g, 9.21 mmol) in 1,4-dioxane (70 mL) was added (tributylstannyl)methanol (3.84 g, 11.96 mmol), Pd(PPh3)4 (1.6 g, 1.38 mmol). The resulting mixture was stirred overnight at 100° C. The reaction was then quenched by the addition of NH₄Cl (sat.aq)(100 mL). The resulting solution was extracted with 3×100 mL of ethyl acetate and the organic layers combined, dried over anhydrous sodium sulfate and concentrated under vacuum. The residue was applied onto a silica gel column with ethyl acetate/petroleum ether (4:1). This resulted in 1.2 g (73%) of (2-methyl-1,3-benzothiazol-4-yl)methanol as yellow oil. LCMS (ES, m/z) 180 [M+H]+

### Step 2. 2-methyl-1,3-benzothiazole-4-carbaldehyde

To a solution of oxalic dichloride (1.7 g, 13.39 mmol) in dichloromethane (30 mL) was added DMSO (1.57 g, 20.09 mmol) dropwise at −78° C. The mixture was stirred for 0.5 h at −78° C., then a solution of (2-methyl-1,3-benzothiazol-4-yl)methanol (1.2 g, 6.69 mmol) in dichloromethane (10 mL) was added at −78° C. and stirred for 2 h, at last, TEA (4.06 g, 40.12 mmol, 6.00 equiv) was added at −78° C. The mixture was stirred for 2 h from −78° C. to room temperature. The reaction was then quenched by the addition of 30 mL of brine. The resulting solution was extracted with 3×30 mL of dichloromethane and the organic layers combined and dried over anhydrous sodium sulfate and concentrated under vacuum. This resulted in 0.9 g (76%) of 2-methyl-1,3-benzothiazole-4-carbaldehyde as a yellow solid. LCMS (ES, m/z) 178 [M+H]+

### Step 3. 2-hydroxy-2-(2-methyl-1,3-benzothiazol-4-yl)acetonitrile

To a mixture of 2-methyl-1,3-benzothiazole-4-carbaldehyde (900 mg, 5.08 mmol) in dichloromethane (15 mL) was added TMSCN (1.51 g, 15.22 mmol) and ZnI2 (162 mg, 0.51 mmol). The mixture was stirred for 2 h at 25° C. The reaction was then quenched by the addition of brine, extracted with 3×30 mL of dichloromethane and the organic layers combined and dried over anhydrous sodium sulfate and concentrated under vacuum. This resulted in 0.8 g (65%) of 2-hydroxy-2-(2-methyl-1,3-benzothiazol-4-yl)acetonitrile as light yellow oil. LCMS (ES, m/z) 205 [M+H]⁺

### Step 4. 2-hydroxy-2-(2-methyl-1,3-benzothiazol-4-yl)acetate

To a mixture of 2-hydroxy-2-(2-methyl-1,3-benzothiazol-4-yl)acetonitrile (800 mg, 3.92 mmol) in methanol (30 mL) was added hydrogen chloride (30 mL, 36.5%), The resulting solution was stirred for 4 h at 60° C. The reaction was then quenched by the addition of 30 mL of brine, extracted with 3×30 mL of ethyl acetate and the organic layers combined, concentrated under vacuum. This resulted in 300 mg of methyl 2-hydroxy-2-(2-methyl-1,3-benzothiazol-4-yl)acetate as light yellow oil. LCMS (ES, m/z) 238 [M+H]⁺

### Step 5. 2-hydroxy-2-(2-methyl-1,3-benzothiazol-4-yl)acetic Acid

To a mixture of 2-hydroxy-2-(2-methyl-1,3-benzothiazol-4-yl)acetic acetate (500 mg, 2.24 mmol) in tetrahydrofuran (3 mL). was added a solution of lithium hydroxide (253 mg, 10.56 mmol) in water (3 mL), The resulting solution was stirred overnight at room temperature. The resulting solution was diluted with 30 mL of H₂O. The resulting solution was extracted with 3×10 mL of ether and the aqueous layers combined. The pH value of the solution was adjusted to 5 with HCl (4M, aq). The resulting solution was extracted with 3×30 mL of ethyl acetate and the organic layers combined and dried over anhydrous sodium sulfate and concentrated under vacuum. This resulted in 300 mg (58%) of 2-hydroxy-2-(2-methyl-1,3-benzothiazol-4-yl)acetic acid as a light yellow solid. LCMS (ES, m/z) 224 [M+H]⁺

### Step 6. (2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (First Eluting Isomer) and (2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (Second Eluting Isomer)

To a mixture of 2-hydroxy-2-(2-methyl-1,3-benzothiazol-4-yl)acetic acid (112 mg, 0.49 mmol) and 2-(2,3-dihydro-1,4-benzodioxine-6-sulfonyl)-1H,2H,3H,4H,5H,6H-pyrrolo[3,4-c]pyrrole; trifluoroacetic acid (203 mg, 0.46 mmol) in N,N-dimethylformamide (4 mL), were added DIEA (129 mg, 1.00 mmol), EDCI (105 mg, 0.55 mmol), HOBt (74 mg, 0.55 mmol). The mixture was stirred for 2 h at room temperature. The resulting solution was diluted with water (30 mL), extracted with 3×30 of ethyl acetate and the organic layers combined and concentrated under vacuum. The crude product was purified by Prep-HPLC with the following conditions: Column: XBridge Shield RP18 OBD Column, 5 μm, 19×150 mm; Mobile Phase A: water (0.05% TFA), Mobile Phase B: ACN; Flow rate: 20 mL/min; Gradient: 25% B to 50% B in 10 min; 254 nm; Rt: 8 min.

The two enantiomers were separated by Chiral-Prep-HPLC with the following conditions: Column: CHIRALPAK IF, 2×25 cm, 5 um; Mobile Phase A: DCM-HPLC, Mobile Phase B: MeOH-HPLC; Flow rate: 16 mL/min; Gradient: 50 B to 50 B in 25 min; 220/254 nm; RT1:17.97; RT2:21.34. This resulted in 13.9 mg (6%) of (2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (first eluting isomer) as a white solid. And 10.4 mg (4%) of (2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (second eluting isomer) as a white solid. Absolute stereochemistry was determined.

(2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (first eluting isomer: 1H NMR (400 MHz, CDCl₃) δ 7.81 (d, J=5.8 Hz, 1H), 7.39-7.31 (m, 2H), 7.30-7.25 (m, 2H), 6.99-6.93 (m, 1H), 6.09 (s, 1H), 4.38-4.25 (m, 6H), 4.20-4.02 (m, 4H), 4.00-3.89 (m, 1H), 3.59-3.50 (m, 1H), 2.89 (s, 3H), 1.25 (s, 1H). LCMS (ES, m/z) 514 [M+H]+

(2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-1,3-benzothiazol-4-yl)ethan-1-one (second eluting isomer): 1H NMR (400 MHz, CDCl3) δ 7.81 (d, J=5.8 Hz, 1H), 7.38-7.31 (m, 2H), 7.30-7.25 (m, 2H), 6.99-6.93 (m, 1H), 6.11 (s, 1H), 4.38-4.25 (m, 6H), 4.16-4.02 (m, 4H), 4.00-3.89 (m, 1H), 3.59-3.50 (m, 1H), 2.91 (s, 3H), 1.25 (s, 1H). LCMS (ES, m/z) 514 [M+H]+

### Method W

### (2S)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (First Eluting Isomer); and (2R)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (Second Eluting Isomer); and (2S)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (Third Eluting Isomer); and (2R)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (Fourth Eluting Isomer)

### Example 109-2. 1^{st} Eluting Isomer; Example 109-3. 2^{nd} Eluting Isomer; Example 109-4. 3^{rd} Eluting Isomer; Example 109-5. 4^{th} Eluting Isomer

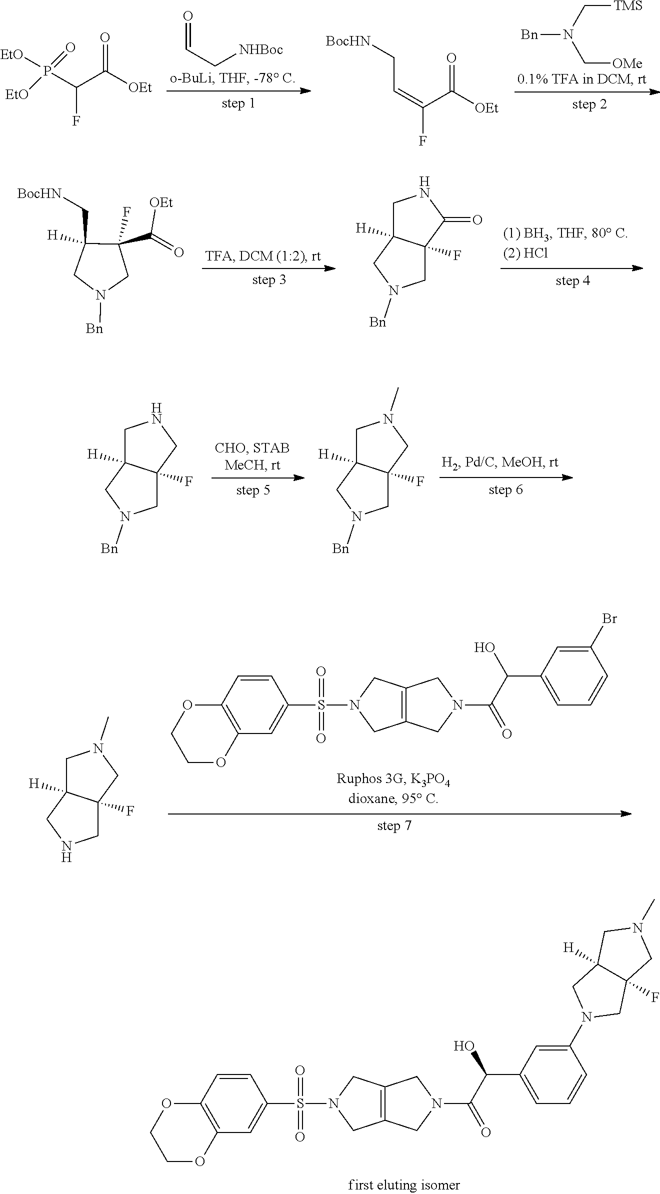

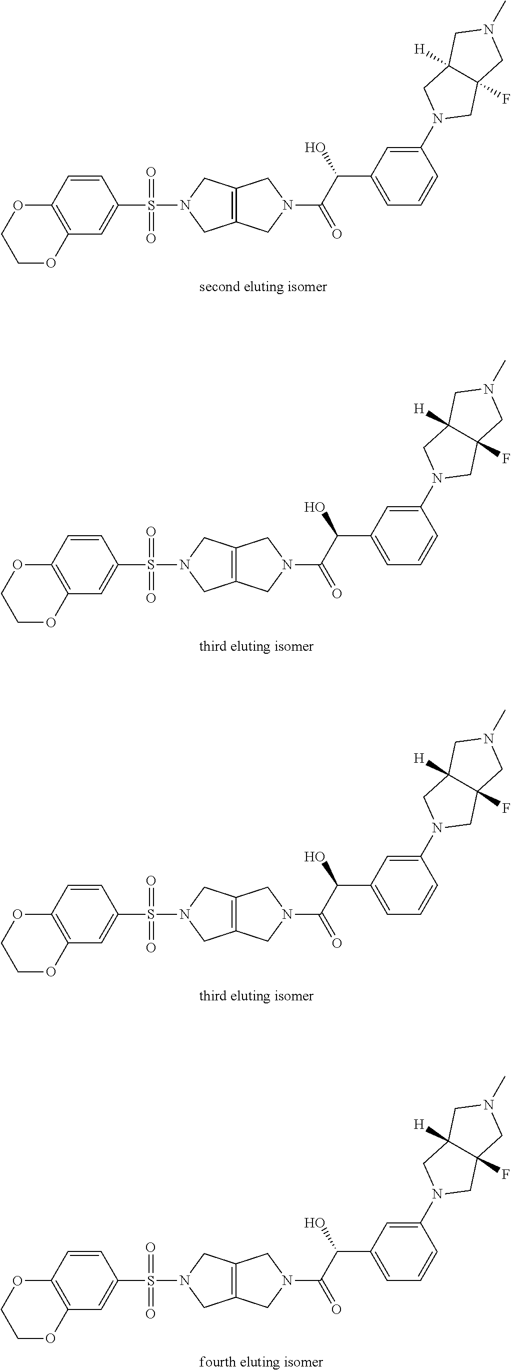 [SMILES: CN1C[C@@H]2CN(c3cccc([C@@H](O)C(=O)N4CC5=C(C4)CN(S(=O)(=O)c4ccc6c(c4)OCCO6)C5)c3)C[C@]2(F)C1 ; CN1C[C@H]2CN(c3cccc([C@H](O)C(=O)N4CC5=C(C4)CN(S(=O)(=O)c4ccc6c(c4)OCCO6)C5)c3)C[C@@]2(F)C1 ; CN1C[C@H]2CN(c3cccc([C@H](O)C(=O)N4CC5=C(C4)CN(S(=O)(=O)c4ccc6c(c4)OCCO6)C5)c3)C[C@@]2(F)C1 ; CN1C[C@H]2CN(c3cccc([C@@H](O)C(=O)N4CC5=C(C4)CN(S(=O)(=O)c4ccc6c(c4)OCCO6)C5)c3)C[C@@]2(F)C1]

### Step 1. Ethyl (2E)-4-{[(tert-butoxy)carbonyl]amino}-2-fluorobut-2-enoate

To a solution of ethyl 2-(diethoxyphosphoryl)-2-fluoroacetate (10 g, 40.5 mmol) in THF (60 mL), was added n-BuLi (16.5 mL, 2.5 M in hexane) at −78° C. and stirred for 1 h, then tert-butyl N-(2-oxoethyl)carbamate (6.57 g, 40.4 mmol) in THF (10 mL) was added. The resulting solution was stirred for 3 h at −78° C. The reaction mixture was poured into saturated sodium bicarbonate (100 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford ethyl (2E)-4-{[(tert-butoxy)carbonyl]amino}-2-fluorobut-2-enoate as light yellow oil (4.1 g, 38.93%). LCMS (ES, m/z): 248 [M+H]⁺.

### Step 2. Ethyl (3S,4R)-1-benzyl-4-({[(tert-butoxy)carbonyl]amino}methyl)-3-fluoropyrrolidine-3-carboxylate

To a solution of ethyl (2E)-4-[[(tert-butoxy)carbonyl]amino]-2-fluorobut-2-enoate (2.5 g, 10.111 mmol) in TFA (100 mL) and DCM (0.1 mL) was added benzyl(methoxymethyl)[(trimethylsilyl)methyl]amine (2.64 mg, 11.1 mmol). The resulting solution was stirred for 16 h at 25° C. and then concentrated under vacuum. The crude product was purified by silica gel chromatography (eluting with 0:100 to 10:90 ethyl acetate/petroleum ether) to afford ethyl (3S,4R)-1-benzyl-4-({[(tert-butoxy)carbonyl]amino}methyl)-3-fluoropyrrolidine-3-carboxylate as light yellow oil (1.2 g, 31.20%). LCMS (ES, m/z): 381 [M+H]⁺.

### Step 3. (3aR,6aS)-5-benzyl-6a-fluoro-octahydropyrrolo[3,4-c]pyrrol-1-one

To a solution of ethyl (3S,4R)-1-benzyl-4-({[(tert-butoxy)carbonyl]amino}methyl)-3-fluoropyrrolidine-3-carboxylate (1.20 g, 3.15 mmol) in DCM (20 mL) was added TFA (10 mL). The resulting solution was stirred for 2 h at 25° C. and then concentrated under vacuum. The reaction mixture was poured into saturated sodium bicarbonate (100 mL) and then extracted with dichloromethane (3×100 mL). The combined organic layers were dried over anhydrous sodium sulfate, filtered, and concentrated under vacuum to afford (3aR,6aS)-5-benzyl-6a-fluoro-octahydropyrrolo[3,4-c]pyrrol-1-one as a white solid (510 mg, 69.02%). LCMS (ES, m/z): 235 [M+H]⁺.

### Step 4. (3aR,6aR)-2-benzyl-3a-fluoro-octahydropyrrolo[3,4-c]pyrrole

To a solution of (3aR,6aS)-5-benzyl-6a-fluoro-octahydropyrrolo[3,4-c]pyrrol-1-one (500 mg, 2.13 mmol) in THF (10 mL) was added 1.0 M BH₃-THF (10.8 mL, 10.7 mmol). The resulting solution was stirred for 16 h at 60° C. and quenched by the addition of 10 mL of 1.0 M HCl and stirred for 2 h at 60° C., concentrated under vacuum. The resulting crude product was purified by silica gel chromatography (eluting with 0:100 to 10:90 methylene chloride/methanol) to afford (3aR,6aR)-2-benzyl-3a-fluoro-octahydropyrrolo[3,4-c]pyrrole as light yellow oil (300 mg, 63.81%). LCMS (ES, m/z): 221 [M+H]⁺.

### Step 5. (3aR,6aR)-2-benzyl-3a-fluoro-5-methyl-octahydropyrrolo[3,4-c]pyrrole

To a solution of (3aR,6aR)-2-benzyl-3a-fluoro-octahydropyrrolo[3,4-c]pyrrole (300 mg, 1.36 mmol) in MeOH (15 mL) was added HCHO (2 mL) and stirred for 30 min, then STAB (865 mg, 4.09 mmol) was added. The resulting solution was stirred for 16 h at 25° C. and then concentrated under vacuum. The resulting crude product was purified by silica gel chromatography (eluting with 0:100 to 10:90 methylene chloride/methanol) to afford (3aR,6aR)-2-benzyl-3a-fluoro-5-methyl-octahydropyrrolo[3,4-c]pyrrole as colorless oil (220 mg, 68.94%). LCMS (ES, m/z): 235 [M+H]⁺.

### Step 6. (3aS,6aS)-3a-fluoro-2-methyl-octahydropyrrolo[3,4-c]pyrrole

To a solution of (3aR,6aR)-2-benzyl-3a-fluoro-5-methyl-octahydropyrrolo[3,4-c]pyrrole (220 mg, 0.939 mmol) in MeOH (15 mL), was added Pd/C (21.9 mg) under hydrogen. The resulting solution was stirred for 3 h at 25° C. The reaction mixture was filtered and concentrated to afford (3aS,6aS)-3a-fluoro-2-methyl-octahydropyrrolo[3,4-c]pyrrole as light yellow oil (100 mg, 73.87%). LCMS (ES, m/z): 145 [M+H]⁺.

### Step 7. (2S)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (First Eluting Isomer)

### (2R)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (Second Eluting Isomer)

### (2S)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (Third Eluting Isomer)

### (2R)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (Fourth Eluting Isomer)

To a solution of (3aS,6aS)-3a-fluoro-2-methyl-octahydropyrrolo[3,4-c]pyrrole (85 mg, 0.59 mmol) in toluene (15 mL), was added RuPhos (27.5 mg, 0.06 mmol), RuPhos Palladacycle Gen.3 (49.3 mg, 0.06 mmol), 2-(3-bromophenyl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (307 mg, 1.77 mmol) and cesium carbonate (577 mg, 1.77 mmol). The resulting solution was stirred for 16 h at 100° C. and cooled to room temperature. The reaction mixture was poured into water (50 mL) and then extracted with ethyl acetate (3×50 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH3CN (30% to 60% over 7 min); Flow rate: 25 mL/min; Detector: UV 254 nm; Rt: 6.5 min). The product fractions were concentrated under vacuum. The two enantiomers were further separated by Chiral Prep-HPLC (Column: CHIRALPAK IE, 5 μm, 20×250 mm; Mobile Phase, A: MTBE (containing 0.2% IPA) and B: MeOH (keep 50% B over 30 min); Flow rate: 13 mL/min; Detector: UV 254/220 nm; M1, 16.249 min; M2, 23.328 min). From M1: Column: CHIRAL ART Amylose-SA S-5 um, 250×20 mm; Mobile Phase A: CO₂: 65, Mobile Phase B: MeOH:DCM=1:1(2 nM NH₃-MEOH): 35; Flow rate: 50 mL/min; 220 nm; 0A: RT:14.53 min; 0B: RT:16.5 min. From M2: Column: Chiralpak IA, 20×250 mm, 5 um; Mobile Phase A: MTBE (containing 0.2% IPA), Mobile Phase B: EtOH; Flow rate: 12 mL/min; Gradient: 50 B to 50 B in 40 min; 220/254 nm; OC: RT: 24.603 min; OD: RT: 32.591 min. The product fractions were concentrated and lyophilized to afford (2S)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one as a white solid (1.3 mg, 0.38%). (2R)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one as a white solid (2.0 mg, 0.58%). (2S)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one as a white solid (5.7 mg, 1.6%). (2R)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one as a white solid (5.3 mg, 1.5%).

(2S)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (first eluting isomer): ¹H NMR (400 MHz, DMSO-d₆) δ: 7.28-7.23 (m, 2H), 7.17-7.13 (m, 1H), 7.06 (d, J=8.4 Hz, 1H), 6.70-6.68 (m, 2H), 6.63-6.61 (m, 1H), 5.52 (d, J=6.8 Hz, 1H), 5.07 (d, J=6.4 Hz, 1H), 4.33-4.29 (m, 4H), 4.26-4.22 (m, 1H), 4.14-4.08 (m, 1H), 4.05-3.96 (m, 5H), 3.95-3.82 (m, 1H), 3.65-3.40 (m, 3H), 3.02-2.96 (m, 1H), 2.95-2.71 (m, 4H), 2.38-2.31 (m, 1H), 2.24 (s, 3H). LCMS (ES, m/z) 585 [M+H]⁺.

(2R)-2-{3-[(3aR,6aR)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (second eluting isomer): ¹H NMR (400 MHz, DMSO-d₆) δ: 7.28-7.23 (m, 2H), 7.17-7.13 (m, 1H), 7.06 (d, J=8.4 Hz, 1H), 6.70-6.68 (m, 2H), 6.63-6.61 (m, 1H), 5.52 (d, J=6.8 Hz, 1H), 5.07 (d, J=6.4 Hz, 1H), 4.33-4.29 (m, 4H), 4.26-4.22 (m, 1H), 4.14-4.08 (m, 1H), 4.05-3.96 (m, 5H), 3.95-3.82 (m, 1H), 3.65-3.40 (m, 3H), 3.02-2.96 (m, 1H), 2.95-2.71 (m, 4H), 2.38-2.31 (m, 1H), 2.26 (s, 3H). LCMS (ES, m/z) 585 [M+H]⁺.

(2S)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (third eluting isomer): ¹H NMR (400 MHz, DMSO-d₆) δ: 7.28-7.23 (m, 2H), 7.17-7.13 (m, 1H), 7.06 (d, J=8.4 Hz, 1H), 6.70-6.68 (m, 2H), 6.63-6.61 (m, 1H), 5.52 (d, J=6.8 Hz, 1H), 5.07 (d, J=6.4 Hz, 1H), 4.33-4.29 (m, 4H), 4.26-4.22 (m, 1H), 4.14-4.08 (m, 1H), 4.05-3.96 (m, 5H), 3.95-3.82 (m, 1H), 3.65-3.40 (m, 3H), 3.02-2.96 (m, 1H), 2.95-2.71 (m, 4H), 2.38-2.31 (m, 1H), 2.26 (s, 3H). LCMS (ES, m/z) 585 [M+H]⁺. LCMS (ES, m/z) 585 [M+H]⁺

(2R)-2-{3-[(3aS,6aS)-3a-fluoro-5-methyl-octahydropyrrolo[3,4-c]pyrrol-2-yl]phenyl}-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (fourth eluting isomer): ¹H NMR (400 MHz, DMSO-d₆) δ: 7.28-7.23 (m, 2H), 7.17-7.13 (m, 1H), 7.06 (d, J=8.4 Hz, 1H), 6.70-6.68 (m, 2H), 6.63-6.61 (m, 1H), 5.52 (d, J=6.8 Hz, 1H), 5.07 (d, J=6.4 Hz, 1H), 4.33-4.29 (m, 4H), 4.26-4.22 (m, 1H), 4.14-4.08 (m, 1H), 4.05-3.96 (m, 5H), 3.95-3.82 (m, 1H), 3.65-3.40 (m, 3H), 3.02-2.96 (m, 1H), 2.95-2.71 (m, 4H), 2.38-2.31 (m, 1H), 2.26 (s, 3H). LCMS (ES, m/z) 585 [M+H]⁺. LCMS (ES, m/z) 585 [M+H]⁺

TABLE 21

| Example(s) | Purification Method |
|---|---|
| 94-1 and 94-2 | Chiral Prep-HPLC (Column: CHIRALPAK IC, 5 μm, 20 × 250 mm; Mobile Phase, A: |
| | DCM and B: MeOH (hold 85% B for 25 min); flow rate: 20 mL/min; Detector: UV |
| | 254/220 nm; RT: 1^{st}, 16.24 min; 2^{nd}, 21.61 min) |
| 94-5 | Prep-HPLC (Column: XBridge Shield RP18 OBD Column, 5 μm, 19 × 150 mm; Mobile |
| | Phase, A: water (containing 0.1% TFA) and B: MeCN (20% to 65% over 4 min, 65% to |
| | 85% over 8 min); Flow rate: 20 mL/min; Detector: UV 254 nm) |
| 94-6 | Prep-HPLC (Column: XBridge Shield RP18 OBD Column, 5 μm, 30 × 150 mm; Mobile |
| | Phase, A: water (containing 10 mmol/L NH4HCO3) and B: CH3CN (35% to 65% over 8 |
| | min); Flow rate: 60 mL/min; Detector: UV 254 nm) |
| 94-7 | Prep-HPLC (Column: XBridge Shield RP18 OBD Column, 5 μm, 19 × 150 mm; Mobile |
| | Phase, A: water (containing 10 mmol/L NH4HCO3 + 0.05% ammonia) and B: CH3CN |
| | (45% to 65% over 8 min); Flow rate: 60 mL/min; Detector: UV 254 nm) |
| 94-8 | Prep-HPLC (Column: SunFire Prep C18, 5 μm, 19 × 150 mm; Mobile Phase, A: water |
| | (containing 0.05% NH4HCO3) and B: MeCN (40% to 80% over 6 min); Flow rate: 15 |
| | mL/min; Detector: UV 254 nm) |
| 94-9 | Prep-HPLC (Column, X Bridge C18, 19 × 150 mm, 5 μm; mobile phase, water |
| | (containing10 mM NH4HCO3 + 0.05% ammonia) and MeCN (40% to 50% MeCN in 8 |
| | min); Flow rate: 15 mL/min; Detector, 254 nm) |
| 94-12 | Prep-HPLC (Column: Waters XBridge RP18, 19 × 150 mm, 5 μm; mobile phase: water |
| | (containing 10 mM NH4HCO3 + 0.05% ammonia) and MeCN (40% to 50% MeCN over 8 |
| | min); Flow rate: 20 mL/min, Detector: UV 254 nm) |
| 94-13 | Prep-HPLC (Column: XBridge Prep C18 OBD Column, 19 × 150 mm 5 μm; Mobile Phase |
| | A: Waters (10 mmoL/L NH4HCO3), Mobile Phase B: ACN; Flow rate: 20 mL/min; |
| | Gradient: 40% B to 40% B in 8 min; Detector: UV 254 nm) |
| 94-14 and 94-15 | Chiral Prep-HPLC (Column: Chiralpak IB, 5 μm, 20 × 250 mm; Mobile Phase, A: DCM |
| | and B: MeOH (keep 50% B over 16.5 min); Flow rate: 20 mL/min; Detector: UV 254/220 |
| | nm; Retention time: 1^{st}, 9.696 min; 2^{nd}, 11.262 min) |
| 94-16 | Prep-HPLC (Column: XBridge Prep C18 OBD, 5 μm, 19 × 150 mm; Mobile Phase, A: |
| | water (containing 5 mmol/L ammonia) and B: MeCN (50% to 62% over 8 min); Flow rate: |
| | 25 mL/min; Detector: UV 254/220 nm) |
| 94-17 and 94-18 | Chiral Prep-HPLC (Column: CHIRALPAK IA, 5 μm, 20 × 250 mm; Mobile Phase, A: |
| | MeOH (containing 0.1% DEA) and B: DCM (keep 10% B over 18 min); Flow rate: 15 |
| | mL/min; Detector: UV 254/220 nm; Retention time: 1^{st}, 11.81 min; 2^{nd}, 13.94 min) |
| 94-19 and 94-20 | Chiral Prep-HPLC (Column: Lux 5u Cellulose-4, AXIA Packed, 5 μm, 21.2 × 250 mm; |
| | Mobile Phase, A: hexane and B: EtOH (keep 10% B over 19 min); Flow rate: 20 mL/min; |
| | Detector: UV 254/220 nm; Retention time: 1^{st}, 12.279 min; 2^{nd}, 15.629 min) |
| 94-21 and 94-22 | Chiral Prep-HPLC (Column: CHIRALPAK ID, 5 μm, 20 × 250 mm; Mobile Phase, A: |
| | DCM and B: EtOH (keep 40% B over 12 min); Flow rate: 16 mL/min; Detector: UV |
| | 254/220 nm; Retention time: 1^{st}, 6.506 min; 2^{nd}, 8.913 min) |
| 94-23 and 94-24 | Chiral Prep-HPLC (Column: CHIRALPAK IF, 5 μm, 20 × 250 mm; Mobile Phase, A: |
| | MeOH and B: DCM (keep 50% B over 11 min); Flow rate: 16 mL/min; Detector: UV |
| | 254/220 nm; Retention time: 1^{st}, 7.99 min; 2^{nd}, 9.68 min) |
| 94-25 and 94-26 | Chiral Prep-HPLC (Column: CHIRAL ART Cellulose-SB, 5 μm, 20 × 250 mm; Mobile |
| | Phase, A: DCM and B: EtOH (keep 40% B over 9 min); Flow rate: 20 mL/min; Detector: |
| | UV 254/220 nm; Retention time: 1^{st}, 4.45 min; 2^{nd}, 7.80 min) |
| 94-27 and 94-28 | Chiral Prep-HPLC (Column: CHIRALPAK IF, 5 μm, 20 × 250 mm; Mobile Phase, A: |
| | DCM and B: MeOH (keep 60% B over 18 min); Flow rate: 16 mL/min; Detector: UV |
| | 254/220 nm; Retention time: 1^{st}, 11.23 min; 2^{nd}, 15.39 min) |
| 94-29 and 94-30 | Chiral Prep-HPLC (Column: CHIRALPAK IF, 5 μm, 10 × 460 mm; Mobile Phase, A: |
| | DCM and B: MeOH (containing 0.1% DEA) (keep 50% B over 16 min); Flow rate: 20 |
| | mL/min; Detector: UV 254/220 nm; Retention time: 1^{st}, 9.72 min; 2^{nd}, 11.81 min) |
| 94-31 and 94-32 | Chiral Prep-HPLC (Column: CHIRALPAK IF, 5 μm, 10 460 mm; Mobile Phase, A: DCM |
| | and B: MeOH (containing 0.1% DEA) (keep 50% B over 16 min); Flow rate: 20 mL/min; |
| | Detector: UV 254/220 nm; Retention time: 1^{st}, 17.97 min; 2^{nd}, 21.34 min) |
| 94-33 and 94-34 | Chiral Prep-HPLC (Column: CHIRALPAK IC, 5 μm, 100 × 460 mm; Mobile Phase, A: |
| | MeOH (containing 0.1% DEA) and B: DCM (keep 40% B over 30 min); Flow rate: 20 |
| | mL/min; Detector: UV 254/220 nm; Retention time: 1^{st}, 18.129 min; 2^{nd}, 25.414 min) |
| 94-35 and 94-36 | Chiral Prep-HPLC (Column: CHIRALPAK IF, 5 μm, 100 × 460 mm; Mobile Phase, A: |
| | MeOH (containing 0.1% DEA) and B: DCM (keep 70% B over 16 min); Flow rate: 20 |
| | mL/min; Detector: UV 254/220 nm; Retention time: 1^{st}, 2.182 min; 2^{nd}, 2.988 min) |
| 94-37 and 94-38 | Chiral Prep-HPLC (Column: CHIRALPAK IF, 5 μm, 100 × 460 mm; Mobile Phase, A: |
| | DCM and B: MeOH (containing 0.1% DEA) (keep 80% B over 16 min); Flow rate: 20 |
| | mL/min; Detector: UV 254/220 nm; Retention time: 1^{st}, 22.274 min; 2^{nd}, 26.604 min) |
| 94-39 and 94-40 | Chiral Prep-HPLC (Column: CHIRALPAK IA, 5 μm, 100 × 460 mm; Mobile Phase, A: |
| | MeOH (containing 0.1% DEA) and B: DCM (keep 15% B over 16 min); Flow rate: 20 |
| | mL/min; Detector: UV 254/220 nm; Retention time: 1^{st}, 2.182 min; 2^{nd}, 2.988 min) |
| 94-41 and 94-42 | Chiral Prep-HPLC (Column: CHIRALPAK IA, 5 μm, 20 × 250 mm; Mobile Phase, A: |
| | MeOH and B: DCM (keep 50% B over 25 min); Flow rate: 16 mL/min; Detector: UV |
| | 254/220 nm; Retention time: 1^{st}, 10.3 min; 2^{nd}, 15.9 min) |
| 94-43 and 94-44 | Chiral Prep-HPLC (Column: CHIRALPAK IE, 2 cm × 25 cm, 5 μm; Mobile Phase A: |
| | MeOH, Mobile Phase B: DCM; Flow rate: 20 mL/min; Gradient: 20 B to 20 B over 30 |
| | min; Detector: UV 254/220 nm; Retention time: 1^{st}, 16.02 min; 2^{nd}, 20.398 min) |
| 94-45 and 94-46 | Chiral Prep-HPLC (Column: CHIRALPAK IF, 5 μm, 20 × 250 mm; Mobile Phase, A: |
| | MeOH (containing 0.1% DEA) and B: DCM (keep 50% B over 10 min); Detector: UV |
| | 254/220 nm; Retention time: 1^{st}, 15.495 min; 2^{nd}, 24.488 min) |
| 94-47 and 94-48 | Chiral Prep-HPLC (Column: CHIRALPAK IF, 5 μm, 20 × 250 mm; Mobile Phase, A: |
| | MeOH (containing 0.1% DEA) and B: DCM (keep 20% B over 23 min); Detector: UV |
| | 254/220 nm; Retention time: 1^{st}, 13.562 min; 2^{nd}, 15.721 min) |
| 94-49 and 94-50 | Chiral Prep-HPLC (Column: CHIRALPAK IA, 5 μm, 20 × 250 mm; Mobile Phase, A: |
| | MeOH; B: DCM (keep 20% B over 24 min); Flow rate: 17 mL/min; Detector: UV 254/220 |
| | nm; Retention time: 1^{st}, 17.3 min; 2^{nd}, 20.4 min) |
| 94-51 and 94-52 | Chiral Prep-HPLC (Column: CHIRALPAK IC, 5 μm, 20 × 250 mm; Mobile Phase, A: |
| | MeOH and B: DCM (keep 30% B over 25 min); Detector: UV 254/220 nm; Retention |
| | time: 1^{st}, 21.424 min; 2^{nd}, 27.827 min) |
| 94-55 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 94-56 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 95-1 and 95-2 | CHIRALPAK IC, 5 μm, 20 × 250 mm; Mobile Phase, A: MTBE (containing 0.1% DEA) |
| | and B: EtOH (keep 50% B over 18 min); Detector: UV 254/220 nm; Retention time: 1^{st}, |
| | 9.54 min; 2^{nd}, 12.96 min). |
| 95-3 and 95-4 | Chiral Prep-HPLC (Column: CHIRALPAK IF, 5 μm, 20 × 250 mm; Mobile Phase, A: |
| | MeOH (containing 0.1% DEA) and B: DCM (keep 50% B over 15 min); Detector: UV |
| | 254/220 nm; Retention time: 1^{st}, 4.88 min; 2^{nd}, 6.81 min) |
| 95-29 and 95-30 | CHIRALPAK IF, 5 μm, 20 × 250 mm; Mobile Phase, A: methanol (containing 0.1% DEA) |
| | and B: DCM (keep 20% B over 13 min); Detector: UV 254/220 nm; Retention time: 1^{st}, |
| | 8.649 min; 0 B, 9.625 min). |
| 95-31 and 95-32 | Chiral Prep-HPLC (Column: CHIRALPAK IF, 5 μm, 20 × 250 mm; Mobile Phase, A: |
| | methanol (containing 0.1% DEA) and B: DCM (keep 20% B over 13 min); Detector: UV |
| | 254/220 nm; Retention time: 1^{st}, 7.96 min; 2^{nd}, 8.97 min) |
| 95-33 and 95-34 | Chiral Prep-HPLC (Column: CHIRAL ART Cellulose-SB, 5 μm, 2 × 25 cm; Mobile Phase |
| | A: MeOH (0.1% DEA), Mobile Phase B: DCM; Flow rate: 20 mL/min; Gradient: 0 B to 0 |
| | B over 20 min; Detector: UV 254/220 nm; Retention time: 1^{st}, 11.752 min; 2^{nd}, 15.221 min) |
| 95-35 and 95-36 | Column: CHIRALPAK IF, 5 μm, 20 × 250 mm; Mobile Phase, A: CO2 and B: EtOH/ACN |
| | (1:1, containing 2 mM NH3); (keep 45 B over 9 min); Flow rate: 45 mL/min; Detector: UV |
| | 254/220 nm; Retention time: 1^{st}, 6.47 min, 2^{nd}: 8.30 min). |
| 95-37 and 95-38 | Prep-HPLC (Column: XBridge Shield RP18 OBD Column, 5 μm, 19 × 150 mm; Mobile |
| | Phase, A: water (containing 10 mmol/L NH4HCO3) and B: CH3CN (30% to 55% over 8 |
| | min); Flow rate: 20 mL/min; Detector: UV 254 nm) |
| 95-39 and 95-40 | Chiral Prep-HPLC (Column: CHIRALPAK IF, 5 μm, 20 × 250 mm; Mobile Phase, A: |
| | methanol (containing 0.1% DEA) and B: DCM (keep 20% B over 25 min); Detector: UV |
| | 254/220 nm; Retention time: 1^{st}, 9.54 min; 2^{nd}, 12.96 min) |
| 95-41 and 95-42 | Chiral Prep-HPLC (Column: CHIRALPAK IF, 5 μm, 20 × 250 mm; Mobile Phase, A: |
| | MeOH (containing 0.1% DEA) and B: DCM (keep 100% A over 18 min); Detector: UV |
| | 254/220 nm; Retention time: 1^{st}: 10.871 min; 2^{nd}: 13.336 min) |
| 95-43 and 95-44 | Chiral Prep-HPLC (Column: CHIRAL ART Cellulose-SB, 5 μm, 20 × 250 mm; Mobile |
| | Phase, A: MeOH (containing 0.1% DEA) and B: DCM (keep 100% A over 22 min); Flow |
| | rate: 20 mL/min; Detector: UV 254/220 nm; Retention time: 1^{st}, 12.88 min; 2^{nd}, 16.003 min) |
| 95-45 | Chiral Prep-HPLC (Column: CHIRALPAK IF, 5 μm, 20 × 250 mm; Mobile Phase, A: |
| | MeOH (containing 0.1% DEA) and B: DCM (keep 0% B over 16 min); Flow rate: 20 |
| | mL/min; Detector: UV 254/220 nm; Retention time: 1^{st}, 9.065 min; 2^{nd}, 10.825 min) |
| 95-47 and 95-48 | Chiral Prep-HPLC (Column: CHIRAL ART Cellulose-SB, 5 μm, 20 × 250 mm; Mobile |
| | Phase, A: MeOH (containing 0.1% DEA) and B: DCM (keep 100% A over 22 min); Flow |
| | rate: 20 mL/min; Detector: UV 254/220 nm; Retention time: 1^{st}, 15.889 min; 2^{nd}, 19.448 min) |
| 95-49 and 95-50 | Chiral Prep-HPLC (Column: CHIRAL ART Cellulose-SB, 5 μm, 20 × 250 mm; Mobile |
| | Phase, A: methanol (containing 0.1% DEA) and B: DCM (keep 20% B over 25 min); |
| | Detector: UV 254/220 nm; Retention time: 1^{st}, 9.538 min; 2^{nd}, 12.958 min) |
| 95-51 and 95-52 | Chiral Prep-HPLC (Column: CHIRAL ART Cellulose-SB, 5 μm, 20 × 250 mm; Mobile |
| | Phase, A: MeOH (containing 0.1% DEA) and B: DCM (keep 100% A over 28 min); Flow |
| | rate: 20 mL/min; Detector: UV 254/220 nm; Retention time: 1^{st}, 16.543 min; 2^{nd}, 20.104 min) |
| 95-53 and 95-54 | Chiral Prep-HPLC (Column: CHIRALPAK IF, 5 μm, 20 × 250 mm; Mobile Phase, A: |
| | MeOH (containing 0.1% DEA) and B: DCM (keep 0% B over 25 min); Detector: UV |
| | 254/220 nm; Retention time: 1^{st}, 11.791 min; 2^{nd}, 18.323 min) |
| 95-55 and 95-56 | Chiral Prep-HPLC (Column: CHIRALPAK IE, 2 × 25 cm, 5 μm; Mobile Phase A: MeOH |
| | (8 mmol/L NH3), Mobile Phase B: DCM; Flow rate: 18 mL/min; Gradient: 50 B to 50 B |
| | over 18 min; Detector: UV 254/220 nm; Retention time: 1^{st}, 9.307 min; 2^{nd}, 14.918 min) |
| Example 95-57 | Prep-HPLC (Column: XBridge Prep C18 OBD Column, 5 μm, 19 × 150 mm; Mobile |
| | Phase, A: water (containing 10 mmol/L NH4HCO3) and B: MeCN (20% to 50% over 7 |
| | min); Flow rate: 20 mL/min; Detector: UV 254/220 nm) |
| 95-58 and 95-59 | Chiral Prep-HPLC (Column: CHIRALPAK IF, 5 μm, 20 × 250 mm; Mobile Phase A: |
| | DCM, Mobile Phase B: MeOH (containing 0.1% DEA) (keep 80% B over 20 min); Flow |
| | rate: 17 mL/min; Detector: UV 254/220 nm; Retention time: 1^{st}, 10.614 min; 2^{nd}, 12.944 |
| | min) |
| 95-60 and 95-61 | Chiral Prep-HPLC (Column: CHIRALPAK IF, 5 μm, 20 × 250 mm; Mobile Phase, A: |
| | methanol (containing 0.1% DEA) and B: DCM (keep 25% B over 17 min); Flow rate: 20 |
| | mL/min; Detector: UV 254/220 nm; Retention time: 1^{st}, 6.912 min; 2^{nd}, 9.942 min) |
| 95-62 and 95-63 | Chiral Prep-HPLC (Column: CHIRALPAK IF, 5 μm, 2 × 25 cm; Mobile Phase A: MeOH |
| | (0.1% DEA), and Mobile Phase B: DCM (keep 25% B over 15 min); Flow rate: 17 |
| | mL/min; Detector: UV 254/220 nm; Rt: 1^{st}: 9.671 min; 2^{nd}: 12.24 min) |
| 95-64 and 95-65 | Chiral Prep-HPLC (Column: CHIRAL ART Cellulose-SB, 5 μm, 20 × 250 mm; Mobile |
| | Phase A: MeOH (containing 0.1% DEA), Mobile Phase B: DCM (keep 100% A over 14 |
| | min); Flow rate: 20 mL/min; Detector: UV 220/254 nm; Retention time: 1^{st}, 9.427 min; 2^{nd}, |
| | 11.568 min) |
| 95-66 and 95-67 | Column: CHIRAL ART Cellulose-SB, 2 × 25 cm, 5 μm; Mobile Phase A: MTBE (2 mM |
| | NH3), Mobile Phase B: EtOH (keep 50% B over 15 min); Flow rate: 18 mL/min; Detector: |
| | 220/254 nm; 1^{st}: 7.932 min; 2^{nd}: 10.552 min). |
| 95-68 and 95-69 | CHIRALPAK IC, 5 μm, 2 × 25 cm; Mobile Phase, A: MeOH (0.1% DEA) and B: DCM |
| | (keep 35% B over 16 min); Flow rate: 20 mL/min; Detector: UV 254/220 nm; Retention |
| | time: 1^{st}, 12.597 min; 2^{nd}, 14.899 min). |
| 95-70 and 95-71 | CHIRAL ART Cellulose-SB, 5 μm, 20 × 250 mm; Mobile Phase, A: MeOH (containing |
| | 0.1% NH3) and B: DCM (keep 0% B over 13 min); Flow rate: 20 mL/min; Detector: UV |
| | 254/220 nm; Retention time: 1^{st}, 9.16 min; 2^{nd}, 10.4 min). |
| 95-72 and 95-73 | Chiral Prep-HPLC (Column: CHIRAL ART Cellulose-SB, 5 μm, 20 × 250 mm; Mobile |
| | Phase, A: MeOH (containing 0.1% DEA) and B: DCM (keep 25% B over 15 min); Flow |
| | rate: 20 mL/min; Detector: UV 254/220 nm; Retention time: 1^{st}: 8.87 min; 2^{nd}: 10.4 min) |
| 95-74 and 95-75 | CHIRAL ART Cellulose-SB, 2 × 25 cm, 5 μm; Mobile Phase A: MTBE (2 mM NH3), |
| | Mobile Phase B: EtOH (keep 20% B over 19 min); Flow rate: 50 mL/min; Detector: |
| | 220/254 nm; 1^{st}: 13.46 min; 2^{nd}: 16.29 min). |
| 95-76 and 95-77 | CHIRALPAK IC, 2 × 25 cm, 5 μm; Mobile Phase A: MTBE (0.1% DEA), Mobile Phase |
| | B: EtOH (keep 30% B over 14 min); Flow rate: 20 mL/min; Detector: 220/254 nm; Rt: 1^{st}: |
| | 8.69 mm, 2^{nd}: 11.31 min). |
| 95-78 and 95-79 | CHIRALPAK IC, 5 μm, 20 × 250 mm; Mobile Phase, A: MTBE (containing 0.1% DEA) |
| | and B: EtOH (keep 30% B over 16 min); Detector: UV 254/220 nm; Retention time: 1^{st}, |
| | 2.182 mm; 2^{nd}, 2.988 min). |
| 95-80 and 95-81 | CHIRALPAK IF, 5 μm, 20 × 250 mm; Mobile Phase, A: MTBE (containing 0.1% DEA) |
| | and B: EtOH (keep 30% B over 25 min); Detector: UV 254/220 nm; Rt: 1^{st}, 1.883 min; 2^{nd}, |
| | 2.396 min). |
| 95-82 and 95-83 | Chiral Prep-HPLC (Column: CHIRALPAK IC, 5 μm, 20 × 250 mm; Mobile Phase, A: |
| | MeOH (containing 0.1% DEA) and B: DCM (keep 100% A over 10 min); Flow rate: 20 |
| | mL/min; Detector: UV 254/220 nm; Retention time: 1^{st}, 7.213 min; 2^{nd}, 8.148 min) |
| 96-1 and 96-2 | Prep-HPLC (Column: XBridge Shield RP18 OBD Column, 5 μm, 19 × 150 mm; Mobile |
| | Phase, A: water (containing 10 mmol/L NH4HCO3) and B: CH3CN (17% to 51% over |
| | 6.33 min); Flow rate: 60 mL/min; Detector: UV 254 nm) |
| 96-3 and 96-4 | Chiral Prep-HPLC (Column: CHIRALPAK IF, 5 μm, 20 × 250 mm; Mobile Phase, A: |
| | MeOH (containing 0.1% DEA) and B: DCM (keep 20% B over 20 min); Detector: UV |
| | 254/220 nm; Retention time: 1^{st}, 11.791 min; 2^{nd}, 18.323 min) |
| 96-5 and 96-6 | Chiral Prep-HPLC (Column: CHIRAL ART Cellulose-SB, 5 μm, 20 × 250 mm; Mobile |
| | Phase, A: methanol (containing 0.1% DEA) and B: DCM (keep 0% B over 14 min); Flow |
| | rate: 20 mL/min; Detector: UV 254/220 nm; Retention time: 1^{st}, 8.82 min; 2^{nd}, 10.702 min) |
| 96-7 | Prep-HPLC (Column: XBridge Shield RP18 OBD Column, 5 μm, 19 × 150 mm; Mobile |
| | Phase, A: water (containing 10 mmol/L NH4HCO3) and B: CH3CN (20% to 35% over 7 |
| | min); Flow rate: 20 mL/min; Detector: UV 254 nm) |
| 96-8 and 96-9 | Chiral Prep-HPLC (Column: CHIRALPAK IF, 5 μm, 20 × 250 mm; Mobile Phase, A: |
| | methanol (containing 0.1% DEA) and B: DCM (keep 20% B over 20 min); Detector: UV |
| | 254/220 nm; Retention time: 1^{st}, 11.312 min; 2^{nd}, 14.721 min) |
| 96-10, 96-11, | Prep-HPLC (Column: XBridge Prep C18 OBD Column 19 × 150 mm, 5 μm; Mobile Phase |
| 96-12, 96-13, | A: water (10 mmoL/L NH4HCO3), Mobile Phase B: CAN (25% B to 44% B over 7 min); |
| 96-14, 96-15, | Flow rate: 20 mL/min; Detector: 254/220 nm). The product fractions were concentrated |
| 96-16, and | under vacuum. The eight enantiomers were further separated by Prep-Chiral PHLC or SFC |
| 96-17 | for several times. Firstly, Mix 1, Mix 2, 7^{th} and 8^{th} were separated by Prep-Chiral HPLC |
| | (CHIRALPAK IC, 2 × 25 cm, 5 μm; Mobile Phase A: MeOH(0.1% DEA), Mobile Phase B: |
| | DCM (keep 20% B over 16 min); Flow rate: 20 mL/min; Detector: 220/254 nm; Rt: |
| | Mix1: 8.464 min; Mix 2: 10.722 min; 7^{th}: 12.192 min; 8^{th}: 13.768 min). Secondly, 1^{st}, Mix 3 |
| | and 4^{th} were separated from Mix 1 by Prep-Chiral HPLC (CHIRALPAK IE, 2 × 25 cm, |
| | 5 μm; Mobile Phase A: MTBE(0.1% DEA), Mobile Phase B: MeOH (keep 15% B over 25 |
| | min); Flow rate: 20 mL/min; Detector: 220/254 nm; Rt: 1^{st}: 13.919 min; Mix 3: 7.894 min; |
| | 4^{th}: 20.885 min). Thirdly, 5^{th} and 6^{th} were separated from Mix 2 by Prep-Chiral HPLC |
| | (Column: CHIRAL ART Cellulose-SB, 2 × 25 cm, 5 μm; Mobile Phase A: MTBE (10 mM |
| | NH3), Mobile Phase B: MeOH (keep 10% B over 20 min); Flow rate: 20 mL/min; |
| | Detector: 220/254 nm; Rt: 5^{th}: 14.317 min; 6^{th}: 17.891 min). Finally, 2^{nd} and 3^{rd} were |
| | separated from Mix 3 by SFC (CHIRALPAK AS-H, 2 × 25 cm, 5 μm; Mobile Phase A: |
| | CO2; Mobile Phase B: MeOH (0.1% DEA) (keep 30% B over 5 min); Flow rate: 45 |
| | mL/min; Detector: 220 nm; Rt: 2^{nd}: 3.64 min; 3^{rd}: 4.69 min). |
| 97-1 and 97-2 | CHIRAL ART Cellulose-SB, 5 μm, 20 × 250 mm; Mobile Phase, A: DCM and B: EtOH |
| | (0.1% DEA) (keep 40% B over 10 min); Detector: UV 254/220 nm; Retention time: 1^{st}, |
| | 6.631 min; 2^{nd}, 8.632 min. |
| 97-3 and 97-4 | Chiral Prep-HPLC (Column: CHIRALPAK IF, 5 μm, 10 460 mm; Mobile Phase, A: |
| | MeOH (containing 0.1% DEA) and B: DCM (keep 70% B over 16 min); Flow rate: 20 |
| | mL/min; Detector: UV 254/220 nm; Retention time: 1^{st}, 2.182 min; 2^{nd}, 2.988 min) |
| 97-5 and 97-6 | Chiral Prep-HPLC (Column: CHIRALPAK IA, 5 μm, 20 × 250 mm; Mobile Phase, A: |
| | MeOH (containing 0.1% DEA) and B: DCM (keep 8% B over 20 min); Flow rate: 14 |
| | mL/min; Detector: UV 254/220 nm; Retention time: 1^{st}, 14.22 min; 2^{nd}, 16.86 min) |
| 97-7 and 97-8 | Prep-HPLC (Column: CHIRALPAK IE, 2 × 25 cm, 5 μm; Mobile Phase A: MTBE |
| | (containing 10 mM NH3), Mobile Phase B: MeOH (Hold 50% B over 10 min); Flow rate: |
| | 16 mL/min; Detector: UV 220/254 nm; 1^{st}: 12.833 min; 2^{nd}: 21.366 min) |
| 97-9 and 97-10 | Prep-HPLC (Column: CHIRALPAK IF, 3 μm, 20 × 250 mm; Mobile Phase, A: methanol |
| | (containing 0.1% DEA) and B: DCM (hold 20% B over 25 min); Detector: UV 254/220 |
| | nm; Retention time: 1^{st}, 3.343 min; 2^{nd}, 4.733 min) |
| 97-11 and 97-12 | Chiral Prep-HPLC (Column: CHIRALPAK IF, 5 μm, 20 × 250 mm; Mobile Phase, A: |
| | MTBE (containing 0.1% DEA) and B: MeOH (keep 50% B over 15 min); Detector: UV |
| | 254/220 nm; Retention time: 1^{st}, 4.673 min; 2^{nd}, 8.258 min) |
| 97-13 and 97-14 | Chiral Prep-HPLC (Column: CHIRALPAK IE, 5 μm, 20 × 250 mm; Mobile Phase, A: |
| | MTBE (containing 0.1% DEA) and B: MeOH; Gradient: keep 50% B over 14 min; Flow |
| | rate: 18 mL/min; Detector: UV 254/220 nm; Retention time: 1^{st}, 9.558 min; 2^{nd}, 11.05 min) |
| 97-15 and 97-16 | Chiral Prep-HPLC (Column: CHIRALPAK IC, 2 × 25 cm, 5 μm; Mobile Phase A: MeCN, |
| | Mobile Phase B: MeOH (0.1% IPA); Flow rate: 20 mL/min; Gradient: keep 10% B over 16 |
| | mm; Detector: UV 254/220 nm; Retention time: 1^{st}, 8.794 min; 2^{nd}, 13.19 min) |
| 97-17 and 97-18 | Chiral Prep-HPLC (Column: CHIRALPAK IF, 5 μm, 20 × 250 mm; Mobile Phase, A: |
| | MeOH (containing 0.1% DEA) and B: DCM; Gradient: keep 55% B over 11 min; |
| | Detector: UV 254/220 nm; Retention time: 1^{st}, 6.057 min; 2^{nd}, 8.405 min) |
| 98-1 and 98-2 | Prep-HPLC (Column: CHIRAL ART Cellulose-SB, 2 × 25 cm, 5 μm; Mobile Phase A: |
| | MTBE (containing 2 mM NH3), Mobile Phase B: EtOH (Hold 30% B over 10 min); Flow |
| | rate: 20 mL/min; Detector: 220/254 nm; RT: 1^{st}: 6.953 min; 2^{nd}: 8.329 min) |
| 99-2 and 99-3 | Chiral Prep-HPLC (Column: CHIRALPAK IC, 5 μm, 20 × 250 mm; Mobile Phase, A: |
| | MeOH (containing 0.1% DEA) and B: DCM (keep 50% B over 15 min); Flow rate: 20 |
| | mL/min; Detector: UV 254/220 nm; Retention time: 1^{st}, 8.537 min; 2^{nd}, 12.174 min) |
| 99-4 and 99-5 | CHIRALPAK IC, 2 × 25 cm, 5 μm; Mobile Phase A: MTBE (0.1% DEA), Mobile Phase B: |
| | EtOH; Flow rate: 20 mL/min; Gradient: 30 B to 30 B over 9 min; Detector 220/254 nm; |
| | Retention time: 1^{st}: 5.273 min, 2^{nd}: 6.987 min). |
| 99-6 and 99-7 | Chiral Prep-HPLC (Column: CHIRALPAK IA, 5 μm, 20 × 250 mm; Mobile Phase, A: |
| | MTBE (containing 0.1% DEA) and B: EtOH (keep 0% B over 15 mm); Flow rate: 14 |
| | mL/min; Detector: UV 254/220 nm; Retention time: 1^{st}, 10.23 min; 2^{nd}, 12.913 min) |
| 99-8 and 99-9 | Chiral Prep-HPLC (Column: CHIRALPAK IF, 5 μm, 20 × 250 mm; Mobile Phase, A: |
| | MTBE (containing 0.1% DEA) and B: MeOH (keep 0% B over 17 min); Flow rate: 15 |
| | mL/min; Detector: UV 254/220 nm; Retention time: 1^{st}, 11.326 min; 2^{nd}, 13.972 min) |
| 99-10 and 99-11 | Chiral Prep-HPLC (Column: CHIRALPAK IA, 5 μm, 20 × 250 mm; Mobile Phase, A: |
| | MeOH (containing 0.1% NH3) and B: EtOH (keep 30% B over 27 min); Flow rate: 14 |
| | mL/min; Detector: UV 254/220 nm; Retention time: 1^{st}, 12.853 min; 2^{nd}, 20.024 min) |
| 99-12 and 99-13 | Chiral Prep-HPLC (Column: CHIRALPAK IE, 2 × 25 cm, 5 μm; Mobile Phase A: MTBE |
| | (0.1% DEA), Mobile Phase B: EtOH; Flow rate: 14 mL/min; Gradient: keep 50% B over |
| | 40 min; Detector: UV 254/220 nm; Retention time: 1^{st}, 21.858 min; 2^{nd}, 27.788 min) |
| 99-14 | Prep-HPLC (Column: Sun Fire Prep C18 OBD column, 5 μm, 19 × 150 mm; Mobile |
| | Phase, A: water (containing 0.1% formic acid) and B: MeCN (15% to 70% over 12 min); |
| | Flow rate: 20 mL/min; Detector: UV 254 nm) |
| 99-15 | Prep-HPLC (Column: X Bridge C18 column, 5 μm, 19 × 150 mm; Mobile Phase, A: water |
| | (containing 10 mM NH4HCO3 and 0.05% ammonia) and B: MeCN (20% to 70% over 10 |
| | min); Flow rate: 20 mL/min; Detector: UV 254 nm) |
| 99-16 and 99-17 | Chiral Prep-HPLC (Column: CHIRALPAK IA, 5 μm, 20 × 250 mm; Mobile Phase, A: |
| | hexane and B: iPrOH (keep 50% B over 44 min); Flow rate: 15 mL/min; Detector: UV |
| | 254/220 nm; Retention time: 1^{st}, 23.86 min; 2^{nd}, 35.74 min) |
| 99-18 | Chiral Prep-HPLC (Column: CHIRALPAK IF, 5 μm, 20 × 250 mm; Mobile Phase, A: |
| | MeOH and B: DCM (keep 30% B over 17 min); Flow rate: 15 mL/min; Detector: UV |
| | 254/220 nm; Retention time: 1^{st}, 11.94 min; 2^{nd}, 14.43 min) |
| 100-1 and 100-2 | CHIRAL ART Cellulose-SB, 2 × 25 cm, 5 μm; Mobile Phase A: MTBE (0.1% DEA), |
| | Mobile Phase B: MeOH (keep 50% B over 23 min); Flow rate: 20 mL/min; Detector: |
| | 220/254 nm; 1^{st}: 12.053 min; 2^{nd}: 18.916 min). |
| 100-3 and 100-4 | CHIRAL ART Cellulose-SB, 2 × 25 cm, 5 μm; Mobile Phase A: MTBE (0.1% DEA), |
| | Mobile Phase B: MeOH (keep 50% B over 23 min); Flow rate: 20 mL/min; Detector: |
| | 220/254 nm; 1^{st}: 12.053 min; 2^{nd}: 18.916 min). |
| 100-5 and 100-6 | Chira Prep-HPLC (Column: CHIRALPAK IG, 2 × 25 cm, 5 μm; Mobile Phase A: MTBE |
| | (0.2% IPA), Mobile Phase B: MeOH; Flow rate: 20 mL/min; Gradient: 40% B to 40% B |
| | over 13 min; Detector: UV 254/220 nm; Retention time: 1^{st}, 6.207 min; 2^{nd}, 9.299 min). |
| 101-1 and 101-2 | Chiral Prep-HPLC (Column: CHIRAL ART Cellulose-SB, 5 μm, 20 × 250 mm; Mobile |
| | Phase, A: hexane/DCM (3:1) and B: EtOH (keep 50% B over 10 min); Flow rate: 20 |
| | mL/min; Detector: UV 254/220 nm; Retention time: 1^{st}, 6.201 min; 2^{nd}, 8.07 min) |
| 101-3 and 101-4 | Prep-HPLC (Column: CHIRALPAK IC, 2 × 25 cm, 5 μm; Mobile Phase A: MeOH, |
| | Mobile Phase B: DCM; Flow rate: 20 mL/min; Gradient: kepp 20% B over 14 min; |
| | Detector: UV 254/220 nm; Retention time: 1^{st}, 10.13 min; 2^{nd}, 11.76 min) |
| 101-5 | Prep-HPLC (Column: X Bridge C18 column, 5 μm, 19 × 150 mm; Mobile Phase, A: water |
| | (containing 10 mM NH4HCO3 and 0.05% ammonia) and B: MeCN (40% to 50% over 9 |
| | min); Flow rate: 15 mL/min; Detector: UV 254 nm) |
| 101-6 | Prep-HPLC (Column: X Bridge C18 column, 5 μm, 19 × 150 mm; Mobile Phase, A: water |
| | (containing 10 mM NH4HCO3 and 0.05% ammonia) and B: MeCN (15% to 75% over 10 |
| | min); Flow rate: 15 mL/min; Detector: UV 254 nm) |
| 102-1 | Prep-HPLC (Column: SunFire Prep C18 column, 5 μm, 19 × 150 mm; Mobile Phase, A: |
| | water (containing 0.1% formic acid) and B: MeCN (15% to 70% over 12 min); Flow rate: |
| | 20 mL/min; Detector: UV 254 nm) |
| 102-2 | Prep-HPLC (Column: XBridge Prep C18 OBD, 5 μm, 19 × 150 mm; Mobile Phase, A: |
| | water (containing 5 mmol/L ammonia) and B: MeCN (43% to 49% over 8 min); Flow rate: |
| | 25 mL/min; Detector: UV 254/220 nm) |
| 103-1 and 103-2 | Chiral Prep-HPLC (Column: CHIRALPAK IF, 5 μm, 20 × 250 mm; Mobile Phase, A: |
| | MeOH (containing 0.1% DEA) and B: DCM (keep 10% B over 16 min); Flow rate: 20 |
| | mL/min; Detector: UV 254/220 nm; Retention time: 1^{st}, 17.285 min; 2^{nd}, 21.532 min) |
| 103-3 and 103-4 | Chiral Prep-HPLC (Column: CHIRALPAK IC, 5 μm, 20 × 250 mm; Mobile Phase, A: |
| | MeOH (containing 0.1% DEA) and B: DCM (keep 30% B over 16 min); Flow rate: 16 |
| | mL/min; Detector: UV 254/220 nm; Retention time: 1^{st}, 11.04 min; 2^{nd}, 13.71 min) |
| 103-5 and 103-6 | Chiral Prep-HPLC (Column: CHIRAL ART Cellulose-SB, 5 μm, 20 × 250 mm; Mobile |
| | Phase, A: MTBE and B: EtOH (keep 30% B over 25 min); Detector: UV 254/220 nm; |
| | Retention time: 1^{st}, 11.42 min; 2^{nd}, 16.71 min) |
| 103-7 and 103-8 | Chiral Prep-HPLC (Column: CHIRALPAK IF, 5 μm, 20 × 250 mm; Mobile Phase A: |
| | MTBE (0.1% DEA), Mobile Phase B: MeOH (keep 50% B over 20 min); Detector: UV |
| | 254/220 nm; Retention time: 1^{st}, 12.327 min; 2^{nd}, 17.189 min) |
| 103-9 and 103-10 | Chiral Prep-HPLC (Column: CHIRALPAK IF, 5 μm, 20 × 250 mm; Mobile Phase, A: |
| | MeOH (containing 0.1% DEA) and B: DCM (keep 40% B over 14 min); Detector: UV |
| | 254/220 nm; Retention time: 1^{st}, 9.08 min; 2^{nd}, 12.115 min) |
| 103-11 and 103-12 | CHIRALPAK IF, 5 μm, 2 × 25 cm; Mobile Phase A: hexane/DCM (v:v = 3:1, containing |
| | 0.1% DEA), and Mobile Phase B: MeOH (keep 50% B over 9 min); Flow rate: 20 mL/min; |
| | Detector: 254/220 nm; Retention time: 1^{st}: 13.313 min; 2^{nd}: 15.174 min). |
| 103-17 and 103-18 | Chiral Prep-HPLC (Column: CHIRALPAK IA, 5 μm, 20 × 250 mm; Mobile Phase, A: |
| | methanol (containing 0.1% DEA) and B: DCM (keep 35% B over 15 min); Flow rate: 18 |
| | mL/min; Detector: UV 254/220 nm; Retention time: 1^{st}, 7.06 min; 2^{nd}, 13.10 min) |
| 103-20 and 103-21 | Chiral Prep-HPLC (Column: CHIRALPAK IF, 5 μm, 100 × 460 mm; Mobile Phase, A: |
| | MeOH (containing 0.1% DEA) and B: DCM (keep 50% B over 16 min); Flow rate: 20 |
| | mL/min; Detector: UV 254/220 nm; Retention time: 1^{st}, 7.437 min; 2^{nd}, 8.523 min) |
| 103-22 and 103-23 | Chiral Prep-HPLC (Column: CHIRALPAK IF, 5 μm, 100 × 460 mm; Mobile Phase, A: |
| | DCM, and B: MeOH (containing 0.1% DEA) (keep 75% B over 16 min); Flow rate: 20 |
| | mL/min; Detector: UV 254/220 nm; Retention time: 1^{st}, 2.182 min; 2^{nd}, 2.988 min) |
| 103-24 and 103-25 | Chiral Prep-HPLC (Column: CHIRALPAK IF, 5 μm, 100 × 460 mm; Mobile Phase, A: |
| | DCM and B: MeOH (containing 0.1% DEA) (keep 70% B over 16 min); Flow rate: 20 |
| | mL/min; Detector: UV 254/220 nm; Retention time: 1^{st}, 8.61 min; 2^{nd}, 12.10 min) |
| 103-26 and 103-27 | Chiral Prep-HPLC (Column: CHIRALPAK IA, 5 μm, 20 × 250 mm; Mobile Phase, A: |
| | MTBE (containing 0.2% IPA) and B: MeOH (keep 34% B over 24 min); Flow rate: 20 |
| | mL/min; Detector: UV 254/220 nm; Retention time: 1^{st}, 15.27 min; 2^{nd}, 21.02 min) |
| 103-28 and 103-29 | Chiral Prep-HPLC (Column: CHIRALPAK IF, 2 × 25 cm, 5 μm; Mobile Phase A: DCM, |
| | Mobile Phase B: MeOH; Flow rate: 17 mL/min; Gradient: 50 B to 50 B over 12 min; |
| | Detector: UV 254/220 nm; Retention time: 1^{st}, 8.01 min; 0 B, 10.07 min) |
| 103-30 and 103-31 | Chiral Prep-HPLC (Column: CHIRALPAK IF, 5 μm, 20 × 250 mm; Mobile Phase, A: |
| | MeOH (containing 0.1% DEA) and B: DCM (keep 40% B over 30 min); Detector: UV |
| | 254/220 nm; Retention time: 1^{st}, 10.634 min; 2^{nd}, 12.944 min) |
| 103-32 and 103-33 | Chiral Prep-HPLC (Column: CHIRALPAK IF, 5 μm, 20 × 250 mm; Mobile Phase, A: |
| | MeOH (containing 0.1% DEA) and B: DCM (keep 30% B over 30 min); Detector: UV |
| | 254/220 nm; Retention time: 1^{st}, 13.9 min; 2^{nd}, 22.3 min) |
| 103-34 and 103-35 | Chiral Prep-HPLC (Column: CHIRALPAK IF, 5 μm, 20 × 250 mm; Mobile Phase, A: |
| | MeOH (containing 0.1% DEA) and B: DCM (keep 50% B over 9 min); Detector: UV |
| | 254/220 nm; Retention time: 1^{st}, 6.68 min; 2^{nd}, 8.17 min) |
| 103-36 and 103-37 | Chiral Prep-HPLC (Column: CHIRALPAK IF, 5 μm, 20 × 250 mm; Mobile Phase, A: |
| | MeOH (containing 0.1% DEA) and B: DCM (keep 4% B over 52 min); Detector: UV |
| | 254/220 nm; Retention time: 1^{st}, 20.7 min; 2^{nd}, 31.6 min) |
| 103-38 and 103-39 | Chiral Prep-HPLC (Column: CHIRALPAK IF, 5 μm, 20 × 250 mm; Mobile Phase, A: |
| | MeOH and B: DCM (keep 30% B over 30 min); Detector: UV 254/220 nm; Retention |
| | time: 1^{st}, 8.57 min; 0 B, 15.48 min) |
| 103-40 and 103-41 | Chiral Prep-HPLC (Column: CHIRALPAK IA, 5 μm, 20 × 250 mm; Mobile Phase, A: |
| | MeOH (containing 0.1% DEA) and B: DCM (keep 10% B over 14 min); Flow rate: 20 |
| | mL/min; Detector: UV 254/220 nm; Retention time: 1^{st}, 10.46 min; 2^{nd}, 12.16 min) |
| 103-42 | Prep-HPLC (Column: XBridge Shield RP18 OBD, 5 μm, 19 × 150 mm; Mobile Phase A: |
| | water (10 mmol/L NH4HCO3) and B: MeCN (30% to 65% over 7 min); Flow rate: 60 |
| | mL/min; Detector: UV 254/220 nm) |
| 103-44 and 103-45 | Chiral Prep-HPLC (Column: CHIRALPAK IA, 2 × 25 cm, 5 μm; Mobile Phase A: MeOH, |
| | Mobile Phase B: DCM; Flow rate: 17 mL/min; Gradient: keep 50% B over 20 min; |
| | Detector: UV 254/220 nm; Retention time: 1^{st}, 14.7 min; 2^{nd}, 17.1 min). |
| 103-46 and 103-47 | Chiral Prep-HPLC (Column: CHIRALPAK IG, 2 × 25 cm, 5 μm; Mobile Phase A: MeOH, |
| | Mobile Phase B: DCM; Flow rate: 18 mL/min; Gradient: keep 40% B over 15 min; |
| | Detector: UV 254/220 nm; Retention time: 1^{st}, 7.253 min; 2^{nd}, 10.19 min). |
| 104-1 and 104-2 | Chiral Prep-HPLC (Column: CHIRAL ART Cellulose-SB, 5 μm, 20 × 250 mm; Mobile |
| | Phase, A: MeOH (containing 0.1% IPA) and B: DCM (keep 100% A over 13 min); Flow |
| | rate: 14 mL/min; Detector: UV 254/220 nm; Retention time: 0 A, 7.566 min; 2^{nd}, 8.977 min) |
| 105-1 and 105-2 | Chiral Prep-HPLC (Column: CHIRALPAK IC, 5 μm, 20 × 250 mm; Mobile Phase, A: |
| | MeOH (containing 0.1% DEA) and B: DCM (keep 50% B over 15 min); Flow rate: 18 |
| | mL/min; Detector: UV 254/220 nm; Retention time: 1^{st}, 7.866 min; 2^{nd}, 11.752 min) |
| 106-1 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 106-2 | Prep-HPLC (XBridge Shield RP18 OBD Column, 5 μm, 19 × 150 mm; Mobile Phase, A: |
| | water (containing 10 mmol/L NH4HCO3 and 0.05% ammonia) and B: MeCN (15% to |
| | 60% over 4 min, 60% B to 85% B over 7 min); Flow rate: 15 mL/min; Detector: UV 254/220 nm) |
| 106-3 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 106-4 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 106-5 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 106-6 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 106-8 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 107-1 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 107-2 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (5% to 35% over 6 min); Flow rate: 25 mL/min; Detector: UV 254/220 nm). |
| 107-3 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 107-4 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 108-3 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 109-1 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 109-2, 109-3, | Chiral Prep-HPLC (Column: CHIRALPAK IA, 5 μm, 20 × 250 mm; Mobile Phase A: |
| 109-4, and | MTBE (containing 0.2% IPA) and B: EtOH; Gradient: keep 50% B over 40 min; Flow |
| 109-5 | rate: 12 mL/min; Detector: UV 254/220 nm. |
| 109-6 and 109-7 | Chiral Prep-HPLC (Column: CHIRALCEL OD-H, 5 μm, 20 × 250 mm; Mobile Phase, A: |
| | hexane (containing 0.1% DEA) and B: ethanol (keep 50% B over 60 min); Flow rate: 20 |
| | mL/min; Detector: UV 254/220 nm; Retention time: 1^{st}, 31.26 min; 2^{nd}, 45.32 min) |
| 109-8 | Prep-HPLC (Column: XBridge Shield RP18 OBD Column, 5 μm, 30 × 150 mm; Mobile |
| | Phase, A: water (containing 10 mmol/L NH4HCO3) and B: MeCN (25% to 50% over 8 |
| | min); Flow rate: 60 mL/min; Detector: UV 254 nm) |
| 109-9 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (5% to 35% over 6 min); Flow rate: 25 mL/min; Detector: UV 254/220 nm). |
| 109-10 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 109-11 | Prep-HPLC (Column: XBridge Prep C18 OBD, 5 μm, 19 × 50 mm; Mobile Phase, A: |
| | Water with 0.1% Ammonium Hydroxide and B: Acetonitrile with 0.1% Ammonium |
| | Hydroxide (25% to 65% over 6 min); Flow rate: 25 mL/min; Detector: UV 254/220 nm). |
| 110-1 and 110-2 | Prep-HPLC (Column: XBridge Shield RP18 OBD Column, 5 μm, 19 × 150 mm; Mobile |
| | Phase, A: water (containing 10 mmol/L NH4HCO3) and B: MeCN (25% to 65% over 7 |
| | min); Flow rate: 20 mL/min; Detector: UV 254 nm) |
| 111-1 and 111-2 | Chiral Prep-HPLC (Column: CHIRALPAK IF, 2 × 25 cm, 5 μm; Mobile Phase A: DCM, |
| | Mobile Phase B: MeOH; Flow rate: 17 mL/min; Gradient: 50 B to 50 B over 12 min; |
| | Detector: UV 254/220 nm; Retention time: 1^{st}, 8.01 min; 2^{nd}, 10.07 min) |
| 112-1 and 112-2 | Chiral Prep-HPLC (Column: CHIRALPAK IF, 5 μm, 20 × 250 mm; Mobile Phase, A: |
| | MeOH (containing 0.1% DEA) and B: DCM (keep 40% B over 50 min); Flow rate: 15 |
| | mL/min; Detector: UV 254/220 nm; Retention time: 1^{st}, 19.223 min; 2^{nd}, 29.404 min) |
| 112-3 and 112-4 | Chiral Prep-HPLC (Column: CHIRALPAK IG, 5 μm, 20 × 250 mm; Mobile Phase, A: |
| | MeOH (containing 0.1% DEA) and B: DCM; Gradient: keep 30% B in 18 min; Flow rate: |
| | 20 mL/min; Detector: UV 254/220 nm; Retention time: 1^{st}, 11.142 min; 2^{nd}, 14.7 min) |
| 112-5 and 112-6 | Chiral Prep-HPLC (Column: CHIRALPAK IE, 5 μm, 20 × 250 mm; Mobile Phase A: |
| | MeOH (8 mmol/L NH3), Mobile Phase B: DCM; Gradient: keep 50% B over 35 min; Flow |
| | rate: 14 mL/min; Detector: UV 254/220 nm; Retention time: 1^{st}, 17.887 min; 2^{nd}, 27.313 min) |
| 113-1 | Prep-HPLC (Waters SunFire C18, 5 um, 19 × 50 mm; Mobile Phase, A: Water with 0.1% |
| | Ammonium Hydroxide and B: Acetonitrile with 0.1% Ammonium Hydroxide (45% to |
| | 85% over 6 min); Flow rate: 25 mL/min; Detector: UV 254/220 nm). |
| 114-1 and 114-2 | Prep-HPLC (Column: CHIRALPAK IE, 2 × 25 cm, 5 μm; Mobile Phase A: MTBE (0.2% |
| | IPA), Mobile Phase B: MeOH; Flow rate: 14 mL/min; Gradient: 50% B to 50% B over 40 |
| | min; Detector: UV 254/220 nm; Retention time: 1^{st}, 22.759 min; 2^{nd}, 34.952 min) |
| 114-3 | Prep-HPLC (Waters SunFire C18, 5 um, 19 × 50 mm; Mobile Phase, A: Water with 0.1% |
| | Ammonium Hydroxide and B: Acetonitrile with 0.1% Ammonium Hydroxide (45% to |
| | 85% over 6 min); Flow rate: 25 mL/min; Detector: UV 254/220 nm). |
| 114-4 | Prep-HPLC (Waters SunFire C18, 5 um, 19 × 50 mm; Mobile Phase, A: Water with 0.1% |
| | Ammonium Hydroxide and B: Acetonitrile with 0.1% Ammonium Hydroxide (25% to |
| | 65% over 6 min); Flow rate: 25 mL/min; Detector: UV 254/220 nm). |
| 114-5 | Prep-HPLC (Waters SunFire C18, 5 um, 19 × 50 mm; Mobile Phase, A: Water with 0.1% |
| | Ammonium Hydroxide and B: Acetonitrile with 0.1% Ammonium Hydroxide (5% to 35% |
| | over 6 min); Flow rate: 25 mL/min; Detector: UV 254/220 nm). |

As set forth in Tables 22 and 23, IC₅₀ values are defined as follows: ≤25 μM and >2 μM (+); ≤2 μM and >0.2 μM (++); ≤0.2 μM and >0.05 μM (+++); ≤0.05 μM and >0.001 μM (++++); and not tested (−−), based upon the Biochemical Assay of Example 1.

In Tables 1 and 22, absolute stereochemistry has not been determined for some Examples. Accordingly, assignment ofany Examples as the “R” or “S” stereoisomer is arbitrary, unless otherwise noted. In some cases, Examples are labeled with “1^{st} eluting isomer” ˜, “2^{nd} eluting isomer”, etc. based on the purification method used to separate the stereoisomers (see Table 21).

TABLE 22

| | IC₅₀ | Inter- | Synth. | | MS m/z | |
|---|---|---|---|---|---|---|
| Ex. | (μM) | mediates | Method | Chemical Name | [M + H]⁺ | ¹H NMR |
| 94-1 | ++ | 2-1, 43-1 | A | (2S)-1-(5-{2H,3H- | 497.1 | (CDCl₃, 400 MHz) δ |
| (1^{st} | | | | [1,4]dioxino[2,3-b]pyridine-7- | | (ppm): 8.29 (d, J = 2.4 |
| eluting | | | | sulfonyl}-1H,2H,3H,5H- | | Hz, 1H), 7.53-7.40 (m, |
| isomer) | | | | pyrrolo[3,4-c]pyrrol-2-yl)-2- | | 2H), 7.30-7.28 (m, |
| 94-2 | + | | | hydroxy-2-(2-methyl-1,3- | | 2H), 6.92 (s, 1H), 6.82 |
| (2^{nd} | | | | benzoxazol-4-yl)ethan-1-one; | | (s, 1H), 5.83 (d, J = 6.4 |
| eluting | | | | (2R)-1-(5-{2H,3H- | | Hz, 1H), 4.70-4.68 (m, |
| isomer) | | | | [1,4]dioxino[2,3-b]pyridine-7- | | 2H), 4.60-4.43 (m, |
| | | | | sulfonyl}-1H,2H,3H,5H- | | 4H), 4.28-4.26 (m, |
| | | | | pyrrolo[3,4-c]pyrrol-2-yl)-2- | | 2H), 3.91-3.89 (m, |
| | | | | hydroxy-2-(2-methyl-1,3- | | 1H), 2.68 (s, 3H). |
| | | | | benzoxazol-4-yl)ethan-1-one | | |
| 94-5 | +++ | 2-2, 54-13 | A | 1-[5-(2,3-dihydro-1,4- | 483.3 | (CD₃OD, 300 MHz) δ |
| | | | | benzodioxine-6-sulfonyl)- | | (ppm): 8.26 (s, 1H), |
| | | | | 1H,2H,3H,4H,5H,6H-pyrrolo[3,4- | | 7.18-7.53 (m, 5H), |
| | | | | c]pyrrol-2-yl]-2-hydroxy-2-(1H- | | 6.96-6.99 (m, 1H), 5.58 |
| | | | | indazol-4-yl)ethan-1-one | | (s, 1H), 4.24-4.31 (m, |
| | | | | | | 6H), 3.92-4.05 (m, |
| | | | | | | 5H), 3.70-3.79 (m, |
| | | | | | | 1H). |
| 94-6 | ++++ | 2-2, 71, | A | (2S)-1-[5-(2,3-dihydro-1,4- | 500.1 | (CD₃OD, 300 MHz) δ |
| | | 54-10 | | benzodioxine-6-sulfonyl)- | | (ppm): 7.36-7.28 (m, |
| | | | | 1H,2H,3H,4H,5H,6H-pyrrolo[3,4- | | 2H), 7.08-6.99 (m, |
| | | | | c]pyrrol-2-yl]-2-(3,4-dihydro-2H- | | 1H), 6.73-6.58 (m, |
| | | | | 1,4-benzoxazin-6-yl)-2- | | 3H), 5.03 (s, 1H), 4.88- |
| | | | | hydroxyethan-1-one; or (2R)-1-[5- | | 4.85 (m, 2H), 4.47-4.00 |
| | | | | (2,3-dihydro-1,4-benzodioxine-6- | | (m, 13H), 3.85-3.72 |
| | | | | sulfonyl)-1H,2H,3H,4H,5H,6H- | | (m, 1H). |
| | | | | pyrrolo[3,4-c]pyrrol-2-yl]-2-(3,4- | | |
| | | | | dihydro-2H-1,4-benzoxazin-6-yl)- | | |
| | | | | 2-hydroxyethan-1-one | | |
| 94-7 | ++ | 39-1 | A | (2S)-1-(5-{2H,3H- | 444.1 | (DMSO-d₆, 300 MHz) |
| | | | | [1,4]dioxino[2,3-b]pyridine-7- | | δ (ppm): 8.10 (d, J = |
| | | | | sulfonyl}-1H,2H,3H,4H,5H,6H- | | 2.1 Hz, 1H), 7.59 (d, |
| | | | | pyrrolo[3,4-c]pyrrol-2-yl)-2- | | J = 2.1 Hz, 1H), 7.29- |
| | | | | hydroxy-2-phenylethan-1-one; or | | 7.32 (m, 5H), 5.13 (s, |
| | | | | (2R)-1-(5-{2H,3H- | | 1H), 4.45 (br s, 2H), |
| | | | | [1,4]dioxino[2,3-b]pyridine-7- | | 4.26 (br s, 3H), 4.09- |
| | | | | sulfonyl}-1H,2H,3H,4H,5H,6H- | | 3.76 (m, 7H). |
| | | | | pyrrolo[3,4-c]pyrrol-2-yl)-2- | | |
| | | | | hydroxy-2-phenylethan-1-one | | |
| 94-8 | ++ | — | A | (2S)-1-[2-(2,3-dihydro-1,4- | 472.0 | (DMSO-d₆, 300 MHz) |
| | | | | benzodioxine-6-sulfonyl)- | | δ (ppm): 3.73 (s, 3H), |
| | | | | 2H,4H,5H,6H-pyrrolo[3,4- | | 4.29-4.76 (m, 8H), |
| | | | | c]pyrazol-5-yl]-2-hydroxy-2-(3- | | 5.24-5.28 (m, 1H), |
| | | | | methoxyphenyl)ethan-1-one; or | | 5.73-5.75 (m, 1H), |
| | | | | (2R)-1-[2-(2,3-dihydro-1,4- | | 6.84-7.45 (m, 7H), 8.21 |
| | | | | benzodioxine-6-sulfonyl)- | | (d, J = 7.8 Hz, 1H). |
| | | | | 2H,4H,5H,6H-pyrrolo[3,4- | | |
| | | | | c]pyrazol-5-yl]-2-hydroxy-2-(3- | | |
| | | | | methoxyphenyl)ethan-1-one | | |
| 94-9 | +++ | 72, 77 | A | (2S)-1-[2-(2,3-dihydro-1,4- | 499.3 | (DMSO-d₆, 300 MHz) |
| | | | | benzodioxine-6-sulfonyl)- | | δ (ppm): 8.22-8.20 (m, |
| | | | | 2H,4H,5H,6H-pyrrolo[3,4- | | 1H), 7.47-7.36 (m, |
| | | | | c]pyrazol-5-yl]-2-(3,4-dihydro- | | 2H), 7.13-7.04 (m, |
| | | | | 2H-1,4-benzoxazin-6-yl)-2- | | 1H), 6.59-6.47 (m, |
| | | | | hydroxyethan-1-one; or (2R)-1-[2- | | 3H), 5.79 (s, 1H), 5.38- |
| | | | | (2,3-dihydro-1,4-benzodioxine-6- | | 5.35 (m, 1H), 5.07-5.03 |
| | | | | sulfonyl)-2H,4H,5H,6H- | | (m, 1H), 4.74-4.23 (m, |
| | | | | pyrrolo[3,4-c]pyrazol-5-yl]-2-(3,4- | | 8H), 4.09-4.07 (m, |
| | | | | dihydro-2H-1,4-benzoxazin-6-yl)- | | 2H), 3.22 (s, 2H). |
| | | | | 2-hydroxyethan-1-one | | |
| 94-12 | +++ | 2-2 | A | (2S)-1-[5-(2,3-dihydro-1,4- | 443.2 | (DMSO-d₆, 300 MHz) |
| | | | | benzodioxine-6-sulfonyl)- | | δ (ppm): 7.35-7.24 (m, |
| | | | | 1H,2H,3H,4H,5H,6H-pyrrolo[3,4- | | 7H), 7.07-7.04 (m, |
| | | | | c]pyrrol-2-yl]-2-hydroxy-2- | | 1H). 5.65-5.63 (m, |
| | | | | phenylethan-1-one | | 1H), 5.17-5.15 (m, |
| | | | | | | 1H), 4.32-4.30 (m, |
| | | | | | | 5H), 4.07-3.93 (m, |
| | | | | | | 7H). |
| 94-13 | ++ | 70 | A | (2S)-1-{5-[(8-fluoro-2,3-dihydro- | 461.3 | (DMSO-d₆, 300 MHz) |
| | | | | 1,4-benzodioxin-6-yl)sulfonyl]- | | δ (ppm): 7.36-7.20 (m, |
| | | | | 1H,2H,3H,4H,5H,6H-pyrrolo[3,4- | | 6H), 6.90-6.86 (m, |
| | | | | c]pyrrol-2-yl}-2-hydroxy-2- | | 1H), 5.66 (s, 1H), 5.18 |
| | | | | phenylethan-1-one | | (s, 1H), 4.36-4.27 (m, |
| | | | | | | 5H), 4.15-3.93 (m, |
| | | | | | | 7H). |
| 94-14 | + | 2-2, 76 | A | 1-[(1S)-2-[5-(2,3-dihydro-1,4- | 510.0 | (DMSO-d₆, 400 MHz) |
| (1^{st} | | | | benzodioxine-6-sulfonyl)- | | δ (ppm): 7.42-7.23 (m, |
| eluting | | | | 1H,2H,3H,4H,5H,6H-pyrrolo[3,4- | | 7H), 7.06 (d, J = 8.4 |
| isomer) | | | | c]pyrrol-2-yl]-2-oxo-1- | | Hz, 1H), 5.80 (s, 1H), |
| 94-15 | ++ | | | phenylethyl]pyrrolidin-2-one; | | 4.33-4.30 (m, 5H), |
| (2^{nd} | | | | 1-[(1R)-2-[5-(2,3-dihydro-1,4- | | 4.09-3.98 (m, 6H), |
| eluting | | | | benzodioxine-6-sulfonyl)- | | 3.69-3.55 (m, 2H), |
| isomer) | | | | 1H,2H,3H,4H,5H,6H-pyrrolo[3,4- | | 2.82-2.76 (m, 1H), |
| | | | | c]pyrrol-2-yl]-2-oxo-1- | | 2.30-2.26 (m, 2H), |
| | | | | phenylethyl]pyrrolidin-2-one | | 1.93-1.80 (m, 2H). |
| 94-16 | ++ | 2-2, 54-14 | A | 1-[5-(2,3-dihydro-1,4- | 478.3 | (DMSO-d₆, 400 MHz) |
| | | | | benzodioxine-6-sulfonyl)- | | δ (ppm): 7.38 (m, 1H). |
| | | | | 1H,2H,3H,4H,5H,6H-pyrrolo[3,4- | | 7.30-7.25 (m, 2H), |
| | | | | c]pyrrol-2-yl]-2-(2-ethyl-1,3- | | 7.07(d, J = 8.4 Hz, 1H), |
| | | | | thiazol-4-yl)-2-hydroxyethan-1- | | 5.60(d, J = 6.8 Hz, 1H), |
| | | | | one | | 5.29(d, J = 7.2 Hz, 1H), |
| | | | | | | 4.33-4.32 (m, |
| | | | | | | 5H), 4.22-4.19 (m, 1H), |
| | | | | | | 4.11-4.03 (m, 6H), |
| | | | | | | 2.95-2.90 (m, 2H), |
| | | | | | | 1.27-1.23 (m, 3H). |
| 94-17 | −− | 74, 39-1 | A | N-[(2S)-1-(5-{2H,3H- | 499.4 | (DMSO-d₆, 400 MHz) |
| (1^{st} | | | | [1,4]dioxino[2,3-b]pyridine-7- | | δ (ppm): 8.29 (s, 1H), |
| eluting | | | | sulfonyl}-1H,2H,3H,4H,5H,6H- | | 8.13 (s, 1H), 7.60 (s, |
| isomer) | | | | pyrrolo[3,4-c]pyrrol-2-yl)-1-oxo- | | 1H), 7.40-7.23 (m, |
| 94-18 | + | | | 2-phenylpropan-2-yl]acetamide; | | 5H), 4.51-4.33 (m, |
| (2^{nd} | | | | N-[(2R)-1-(5-{2H,3H- | | 4H), 4.17-4.15 (m, |
| eluting | | | | [1,4]dioxino[2,3-b]pyridine-7- | | 1H), 4.06-3.96 (m, |
| isomer) | | | | sulfonyl}-1H,2H,3H,4H,5H,6H- | | 5H), 3.81-3.78 (m, |
| | | | | pyrrolo[3,4-c]pyrrol-2-yl)-1-oxo- | | 1H), 3.53-3.50 (m, |
| | | | | 2-phenylpropan-2-yl]acetamide | | 1H), 1.93 (s, 3H), 1.60 |
| | | | | | | (s, 3H). |
| 94-19 | +++ | 39-1, 54-12 | A | (2S)-1-(5-{2H,3H- | 458.3 | (DMSO-d₆, 300 MHz) |
| (1^{st} | | | | [1,4]dioxino[2,3-b]pyridine-7- | | δ (ppm): 8.15 (d, J = |
| eluting | | | | sulfonyl}-1H,2H,3H,4H,5H,6H- | | 2.1 Hz, 1H), 7.62 (d, J = |
| isomer) | | | | pyrrolo[3,4-c]pyrrol-2-yl)-2- | | 2.1 Hz, 1H), 7.25- |
| | | | | | | 7.09 (m, 4H), 5.59 (d, J = |
| 94-20 | + | | | hydroxy-2-(3-methylphenyl)ethan- | | 6.3 Hz, 1H), 5.13 (d, |
| (2^{nd} | | | | 1-one; | | J = 6.0 Hz, 1H), 4.51 |
| eluting | | | | (2R)-1-(5-{2H,3H- | | (s, 2H), 4.33-4.23 (m, |
| isomer) | | | | [1,4]dioxino[2,3-b]pyridine-7- | | 3H), 4.13-3.88 (m, |
| | | | | sulfonyl}-1H,2H,3H,4H,5H,6H- | | 7H), 2.29 (s, 3H). |
| | | | | pyrrolo[3,4-c]pyrrol-2-yl)-2- | | |
| | | | | hydroxy-2-(3-methylphenyl)ethan- | | |
| | | | | 1-one | | |
| 94-21 | +++ | 2-2, 67 | A | N-[(1S)-1-(1,2-benzoxazol-3-yl)- | 525.4 | (DMSO-d₆, 400 MHz) |
| (1^{st} | | | | 2-[5-(2,3-dihydro-1,4- | | δ (ppm): 9.01 (d, J = |
| eluting | | | | benzodioxine-6-sulfonyl)- | | 7.6 Hz, 1H), 7.92 (d, J = |
| isomer) | | | | 1H,2H,3H,4H,5H,6H-pyrrolo[3,4- | | 8.0 Hz, 1H), 7.75 (d, |
| 94-22 | + | | | c]pyrrol-2-yl]-2- | | J = 8.4 Hz, 1H), 7.68- |
| (2^{nd} | | | | oxoethyl]acetamide; | | 7.65 (m, 1H), 7.42-7.39 |
| eluting | | | | N-[(1R)-1-(1,2-benzoxazol-3-yl)- | | (m, 1H), 7.29-7.24 (m, |
| isomer) | | | | 2-[5-(2,3-dihydro-1,4- | | 2H), 7.06 (d, J = 8.4 |
| | | | | benzodioxine-6-sulfonyl)- | | Hz, 1H), 6.14 (d, J = |
| | | | | 1H,2H,3H,4H,5H,6H-pyrrolo[3,4- | | 8.0 Hz, 1H), 4.44-4.40 |
| | | | | c]pyrrol-2-yl]-2- | | (m, 1H), 4.32-4.31 (m, |
| | | | | oxoethyl]acetamide | | 4H), 4.12-4.02 (m, |
| | | | | | | 7H), 1.92 (s, 3H). |
| 94-23 | ++ | 39-1, 54-9 | A | (2S)-2-(6-chloropyridin-2-yl)-1- | 479.2 | (DMSO-d₆, 300 MHz) |
| (1^{st} | | | | (5-{2H,3H-[1,4]dioxino[2,3- | | δ (ppm): 8.17 (d, J = |
| eluting | | | | b]pyridine-7-sulfonyl}- | | 2.1 Hz, 1H), 7.92-7.87 |
| isomer) | | | | 1H,2H,3H,4H,5H,6H-pyrrolo[3,4- | | (m, 1H), 7.65 (d, J = |
| 94-24 | −− | | | c]pyrrol-2-yl)-2-hydroxyethan-1- | | 2.4 Hz, 1H), 7.52 (d, |
| (2^{nd} | | | | one; | | J = 7.5 Hz, 1H), 7.43 (d, |
| eluting | | | | (2R)-2-(6-chloropyridin-2-yl)-1- | | J = 7.8 Hz, 1H), 6.11 |
| isomer) | | | | (5-{2H,3H-[1,4]dioxino[2,3- | | (d, J = 6.9 Hz, 1H), |
| | | | | b]pyridine-7-sulfonyl}- | | 5.26 (d, J = 6.9 Hz, |
| | | | | 1H,2H,3H,4H,5H,6H-pyrrolo[3,4- | | 1H), 4.52-4.51 (m, |
| | | | | c]pyrrol-2-yl)-2-hydroxyethan-1- | | 2H), 4.40-4.26 (m, |
| | | | | one | | 4H), 4.10-3.97 (m, |
| | | | | | | 6H). |
| 94-25 | + | 39-1, 54-11 | A | (2S)-1-(5-{2H,3H- | 475.0 | (DMSO-d₆, 300 MHz) |
| (1^{st} | | | | [1,4]dioxino[2,3-b]pyridine-7- | | δ (ppm): 8.18 (d, J = |
| eluting | | | | sulfonyl}-1H,2H,3H,4H,5H,6H- | | 1.8 Hz, 1H), 7.74-7.65 |
| isomer) | | | | pyrrolo[3,4-c]pyrrol-2-yl)-2- | | (m, 2H), 7.12 (d, J = |
| 94-26 | + | | | hydroxy-2-(6-methoxypyridin-2- | | 7.5 Hz, 1H), 6.72 (d, J = |
| (2^{nd} | | | | yl)ethan-1-one; | | 8.4 Hz, 1H), 5.74 (d, J = |
| eluting | | | | (2R)-1-(5-{2H,3H- | | 7.2 Hz, 1H), 5.22 (d, |
| isomer) | | | | [1,4]dioxino[2,3-b]pyridine-7- | | J = 7.2 Hz, 1H), 4.53- |
| | | | | sulfonyl}-1H,2H,3H,4H,5H,6H- | | 4.35 (m, 6H), 4.14-4.04 |
| | | | | pyrrolo[3,4-c]pyrrol-2-yl)-2- | | (m, 6H), 3.72 (s, 3H). |
| | | | | hydroxy-2-(6-methoxypyridin-2- | | |
| | | | | yl)ethan-1-one | | |
| 94-27 | +++ | 43-1, 39-1 | A | (2S)-1-(5-{2H,3H- | 499.3 | (DMSO-d₆, 400 MHz) |
| (1^{st} | | | | [1,4]dioxino[2,3-b]pyridine-7- | | δ (ppm): 8.16 (d, J = |
| eluting | | | | sulfonyl}-1H,2H,3H,4H,5H,6H- | | 2.4 Hz, 1H), 7.63-7.58 |
| isomer) | | | | pyrrolo[3,4-c]pyrrol-2-yl)-2- | | (m, 2H), 7.33-7.32 (m, |
| | | | | hydroxy-2-(2-methyl-1,3- | | 2H), 5.70 (s, 2H), 4.52- |
| | | | | benzoxazol-4-yl)ethan-1-one | | 4.50 (m, 2H), 4.34-4.32 |
| | | | | | | (m, 3H), 4.09-4.05 (m, |
| | | | | | | 7H), 2.63 (s, 3H). |
| 94-28 | + | 43-1,39-1 | A | (2R)-1-(5-{2H,3H- | 499.3 | (DMSO-d₆, 400 MHz) |
| (2^{nd} | | | | [1,4]dioxino[2,3-b]pyridine-7- | | δ (ppm): 8.16 (d, J = |
| eluting | | | | sulfonyl}-1H,2H,3H,4H,5H,6H- | | 2.4 Hz, 1H), 7.63-7.58 |
| isomer) | | | | pyrrolo[3,4-c]pyrrol-2-yl)-2- | | (m, 2H), 7.33-7.32 (m, |
| | | | | hydroxy-2-(2-methyl-1,3- | | 2H), 5.70 (s, 2H), 4.52- |
| | | | | benzoxazol-4-yl)ethan-1-one | | 4.50 (m, 2H), 4.34-4.32 |
| | | | | | | (m, 3H), 4.09-4.05 (m, |
| | | | | | | 7H), 2.63 (s, 3H). |
| 94-29 | +++ | 45-3, 39-1 | A | (2S)-1-(5-{2H,3H- | 495.1 | (DMSO-d₆, 300 MHz) δ |
| (1^{st} | | | | [1,4]dioxino[2,3-b]pyridine-7- | | (ppm): 8.91-8.89 (m, |
| eluting | | | | sulfonyl}-1H,2H,3H,4H,5H,6H- | | 1H), 8.41-8.38 (m, |
| isomer) | | | | pyrrolo[3,4-c]pyrrol-2-yl)-2- | | 1H), 8.18-8.17 (s, 1H), |
| 94-30 | + | | | hydroxy-2-(quinolin-8-yl)ethan-1- | | 7.95-7.92 (m, 1H), |
| (2^{nd} | | | | one; | | 7.86-7.84 (m, 1H), |
| eluting | | | | (2R)-1-(5-{2H,3H- | | 7.65-7.54 (m, 3H), 6.35 |
| isomer) | | | | [1,4]dioxino[2,3-b]pyridine-7- | | (d, J = 6.8 Hz, 1H), |
| | | | | sulfonyl}-1H,2H,3H,4H,5H,6H- | | 5.86 (d, J = 6.8 Hz, |
| | | | | pyrrolo[3,4-c]pyrrol-2-yl)-2- | | 1H), 4.53-4.51 (m, |
| | | | | hydroxy-2-(quinolin-8-yl)ethan-1- | | 3H), 4.46 (m, 2H), |
| | | | | one | | 4.36-3.95 (m, 7H). |
| 94-31 | ++++ | 2-2, 44-1 | A | (2S)-1-[5-(2,3-dihydro-1,4- | 514.2 | (CDCl₃, 400 MHz) δ |
| (1^{st} | | | | benzodioxine-6-sulfonyl)- | | (ppm): 7.81 (d, J = 5.6 |
| eluting | | | | 1H,2H,3H,4H,5H,6H-pyrrolo[3,4- | | Hz, 1H), 7.39-7.31 (m, |
| isomer) | | | | c]pyrrol-2-yl]-2-hydroxy-2-(2- | | 2H), 7.35-7.25 (m, |
| | | | | methyl-1,3-benzothiazol-4- | | 2H), 6.95 (d, J = 8.4 |
| | | | | yl)ethan-1-one | | Hz, 1H), 6.10 (s, 1H), |
| | | | | | | 4.38-4.25 (m, 6H), |
| | | | | | | 4.22-4.19 (m, 1H), |
| | | | | | | 4.16-4.02 (m, 3H), 4.01 |
| | | | | | | (s, 1H), 3.94 (s, 1H), |
| | | | | | | 3.55-3.53 (m, 1H), 2.89 |
| | | | | | | (s, 3H). |
| 94-32 | ++ | 2-2, 44-1 | A | (2R)-1-[5-(2,3-dihydro-1,4- | 514.2 | (CDCl₃, 400 MHz) δ |
| (2^{nd} | | | | benzodioxine-6-sulfonyl)- | | (ppm): 7.81 (d, J = 5.6 |
| eluting | | | | 1H,2H,3H,4H,5H,6H-pyrrolo[3,4- | | Hz, 1H), 7.39-7.31 (m, |
| isomer) | | | | c]pyrrol-2-yl]-2-hydroxy-2-(2- | | 2H), 7.35-7.25 (m, |
| | | | | methyl-1,3-benzothiazol-4- | | 2H), 6.95 (d, J = 8.4 |
| | | | | yl)ethan-1-one | | Hz, 1H), 6.10 (s, 1H), |
| | | | | | | 4.38-4.25 (m, 6H), |
| | | | | | | 4.22-4.19 (m, 1H), |
| | | | | | | 4.16-4.02 (m, 3H), 4.01 |
| | | | | | | (s, 1H), 3.94 (s, 1H), |
| | | | | | | 3.55-3.53 (m, 1H), 2.89 |
| | | | | | | (s, 3H). |
| 94-33 | ++ | 54-2, 39-1 | A | (2S)-1-(5-{2H,3H- | 495.1 | (DMSO-d₆, 300 MHz) δ |
| (1^{st} | | | | [1,4]dioxino[2,3-b]pyridine-7- | | (ppm): 8.91-8.89 (m, |
| eluting | | | | sulfonyl}-1H,2H,3H,4H,5H,6H- | | 1H), 8.73-8.70 (m, |
| isomer) | | | | pyrrolo[3,4-c]pyrrol-2-yl)-2- | | 1H), 8.13 (s, 1H), 7.98 |
| 94-34 | −− | | | hydroxy-2-(quinolin-5-yl)ethan-1- | | (d, J = 8.4 Hz, 1H), |
| (2^{nd} | | | | one; | | 7.75-7.70 (m, 1H), |
| eluting | | | | (2R)-1-(5-{2H,3H- | | 7.60-7.53 (m, 3H), |
| isomer) | | | | [1,4]dioxino[2,3-b]pyridine-7- | | 5.91-5.76 (m, 2H), |
| | | | | sulfonyl}-1H,2H,3H,4H,5H,6H- | | 4.51-4.49 (m, 2H), |
| | | | | pyrrolo[3,4-c]pyrrol-2-yl)-2- | | 4.37-4.31 (m, 3H), |
| | | | | hydroxy-2-(quinolin-5-yl)ethan-1- | | 4.13-4.06 (m, 3H), 4.00 |
| | | | | one | | (s, 2H), 3.73-3.72 (m, |
| | | | | | | 1H), 3.17 (d, J = 5.1 |
| | | | | | | Hz, 1H). |
| 94-35 | +++ | 45-1, 44-1, | A | (2S)-1-(5-{2H,3H- | 515.2 | (CDCl₃, 400 MHz) δ |
| (1^{st} | | 39-1 | | [1,4]dioxino[2,3-b]pyridine-7- | | (ppm): 8.25 (d, J = 2.0 |
| eluting | | | | sulfonyl}-1H,2H,3H,4H,5H,6H- | | Hz, 1H), 7.87-7.77 (m, |
| isomer) | | | | pyrrolo[3,4-c]pyrrol-2-yl)-2- | | 1H), 7.54 (d, J = 2.4 |
| 94-36 | + | | | hydroxy-2-(2-methyl-1,3- | | Hz, 1H), 7.43-7.33 (m, |
| (2^{nd} | | | | benzothiazol-4-yl)ethan-1-one; | | 2H), 6.13 (s, 1H), 4.55- |
| eluting | | | | (2R)-1-(5-{2H,3H- | | 4.48 (m, 2H), 4.40-4.27 |
| isomer) | | | | [1,4]dioxino[2,3-b]pyridine-7- | | (m, 4H), 4.22-4.01 (m, |
| | | | | sulfonyl}-1H,2H,3H,4H,5H,6H- | | 4H), 3.99-3.96 (m, |
| | | | | pyrrolo[3,4-c]pyrrol-2-yl)-2- | | 1H), 3.59-3.56 (m, |
| | | | | hydroxy-2-(2-methyl-1,3- | | 1H), 2.92 (s, 3H). |
| | | | | benzothiazol-4-yl)ethan-1-one | | |
| 94-37 | ++++ | 44-2, 2-2 | A | (2S)-1-[5-(2,3-dihydro-1,4- | 498.2 | (CDCl₃, 400 MHz,) δ |
| (1^{st} | | | | benzodioxine-6-sulfonyl)- | | (ppm): 7.45-7.43 (m, |
| eluting | | | | 1H,2H,3H,4H,5H,6H-pyrrolo[3,4- | | 1H), 7.35-7.20 (m, |
| isomer) | | | | c]pyrrol-2-yl]-2-hydroxy-2-(2- | | 4H), 6.95 (d, J = 8.4 |
| 94-38 | ++ | | | methyl-1,3-benzoxazol-4-yl)ethan- | | Hz, 1H), 5.74 (s, 1H), |
| (2^{nd} | | | | 1-one; | | 4.38-4.13 (m, 5H), |
| eluting | | | | (2R)-1-[5-(2,3-dihydro-1,4- | | 4.11-4.02 (m, 5H), |
| isomer) | | | | benzodioxine-6-sulfonyl)- | | 3.98-3.92 (m, 1H), |
| | | | | 1H,2H,3H,4H,5H,6H-pyrrolo[3,4- | | 3.56-3.54 (m, 1H), 2.67 |
| | | | | c]pyrrol-2-yl]-2-hydroxy-2-(2- | | (s, 3H). |
| | | | | methyl-1,3-benzoxazol-4-yl)ethan- | | |
| | | | | 1-one | | |
| 94-39 | −− | 52-1, 43-1, | A | (2S)-1-{5-[(5-fluoro-2,3-dihydro- | 516.3 | (CD₃OD, 400 MHz) δ |
| (1^{st} | | 53-1 | | 1,4-benzodioxin-6-yl)sulfonyl]- | | (ppm): 7.54-7.51 (m, |
| eluting | | | | 1H,2H,3H,4H,5H,6H-pyrrolo[3,4- | | 1H), 7.46-7.33 (m, |
| isomer) | | | | c]pyrrol-2-yl}-2-hydroxy-2-(2- | | 3H), 6.85-6.80 (m, |
| 94-40 | −− | | | methyl-1,3-benzoxazol-4-yl)ethan- | | 1H), 5.90 (s, 1H), 4.50- |
| (2^{nd} | | | | 1-one; | | 4.27 (m, 6H), 4.26-4.07 |
| eluting | | | | (2R)-1-{5-[(5-fluoro-2,3-dihydro- | | (m, 5H), 3.99-3.79 (m, |
| isomer) | | | | 1,4-benzodioxin-6-yl)sulfonyl]- | | 1H), 2.66 (s, 3H). |
| | | | | 1H,2H,3H,4H,5H,6H-pyrrolo[3,4- | | |
| | | | | c]pyrrol-2-yl}-2-hydroxy-2-(2- | | |
| | | | | methyl-1,3-benzoxazol-4-yl)ethan- | | |
| | | | | 1-one | | |
| 94-41 | ++ | 57-1, 43-1 | A | (2S)-2-hydroxy-2-(2-methyl-1,3- | 570.4 | (CD₃OD, 400 MHz) δ |
| (1^{st} | | | | benzoxazol-4-yl)-1-{5-[(2,2,3,3- | | (ppm): 7.84-7.73 (m, |
| eluting | | | | tetrafluoro-2,3-dihydro-1,4- | | 2H), 7.55-7.47 (m, |
| isomer) | | | | benzodioxin-6-yl)sulfonyl]- | | 2H), 7.41-7.29 (m, |
| 94-42 | −− | | | 1H,2H,3H,4H,5H,6H-pyrrolo[3,4- | | 2H), 5.84 (s, 1H), 4.41- |
| (2^{nd} | | | | c]pyrrol-2-yl}ethan-1-one; | | 4.32 (m, 1H), 4.24-3.98 |
| eluting | | | | (2R)-2-hydroxy-2-(2-methyl-1,3- | | (m, 6H), 3.90-3.79 (m, |
| isomer) | | | | benzoxazol-4-yl)-1-{5-[(2,2,3,3- | | 1H), 2.63 (s, 3H). |
| | | | | tetrafluoro-2,3-dihydro-1,4- | | |
| | | | | benzodioxin-6-yl)sulfonyl]- | | |
| | | | | 1H,2H,3H,4H,5H,6H-pyrrolo[3,4- | | |
| | | | | c]pyrrol-2-yl}ethan-1-one | | |
| 94-43 | +++ | 49-1, 45-4, | A | (2S)-1-[5-(2,3-dihydro-1,4- | 529.4 | (DMSO-d₆, 300 MHz) |
| (1^{st} | | 2-2 | | benzodioxine-6-sulfonyl)- | | δ (ppm): 8.05-8.03 (m, |
| eluting | | | | 1H,2H,3H,4H,5H,6H-pyrrolo[3,4- | | 1H), 7.32-7.19 (m, |
| isomer) | | | | c]pyrrol-2-yl]-2-hydroxy-2-[2- | | 2H), 7.05-7.03 (m, |
| 94-44 | + | | | (morpholin-4-yl)pyridin-4- | | 1H), 6.78 (s, 1H), 6.63- |
| (2^{nd} | | | | yl]ethan-1-one; | | 6.61(m, 1H), 5.89-5.87 |
| eluting | | | | (2R)-1-[5-(2,3-dihydro-1,4- | | (m, 1H), 5.10-5.09 (m, |
| isomer) | | | | benzodioxine-6-sulfonyl)- | | 1H), 4.32-4.20 (m, |
| | | | | 1H,2H,3H,4H,5H,6H-pyrrolo[3,4- | | 5H), 4.12-4.01 (m, |
| | | | | c]pyrrol-2-yl]-2-hydroxy-2-[2- | | 7H), 3.67-3.59 (m, |
| | | | | (morpholin-4-yl)pyridin-4- | | 4H), 3.47-2.36 (m, |
| | | | | yl]ethan-1-one | | 4H). |
| 94-45 | + | 47-1, 38-1 | A | (2S)-1-(5-{2H,3H- | 499.3 | (DMSO-d₆, 400 MHz) |
| (1^{st} | | | | [1,4]dioxino[2,3-b]pyridine-7- | | δ (ppm): 8.12 (d, J = |
| eluting | | | | sulfonyl}-1H,2H,3H,4H,5H,6H- | | 2.2 Hz, 1H), 7.79 (d, |
| isomer) | | | | pyrrolo[3,4-c]pyrrol-2-yl)-2- | | J = 8.4 Hz, 1H), 7.65 (s, |
| 94-46 | −− | | | hydroxy-2-(3-methyl-1,2- | | 1H), 7.59 (d, J = 2.0 |
| (2^{nd} | | | | benzoxazol-4-yl)ethan-1-one; | | Hz, 1H), 7.35 (dd, J = |
| eluting | | | | (2R)-1-(5-{2H,3H- | | 8.4, 1.2 Hz, 1H), 6.01 |
| isomer) | | | | [1,4]dioxino[2,3-b]pyridine-7- | | (d, J = 6.4 Hz, 1H), |
| | | | | sulfonyl}-1H,2H,3H,4H,5H,6H- | | 5.36 (d, J = 6.4 Hz |
| | | | | pyrrolo[3,4-c]pyrrol-2-yl)-2- | | 1H), 4.48 (dd, J = 5.2 |
| | | | | hydroxy-2-(3-methyl-1,2- | | 2.8 Hz, 2H), 4.33-4.26 |
| | | | | benzoxazol-4-yl)ethan-1-one | | (m, 2H), 4.23 (s, 1H) |
| | | | | | | 4.08-3.934 (m,7H), |
| | | | | | | 2.52 (s, 3H). |
| 94-47 | +++ | 50-1, 2-2 | A | (2S)-2-(1,2-benzoxazol-3-yl)-1-[5- | 484.4 | (DMSO-d₆, 400 MHz) |
| (1^{st} | | | | (2,3-dihydro-1,4-benzodioxine-6- | | δ (ppm): 7.90 (d, J = |
| eluting | | | | sulfonyl)-1H,2H,3H,4H,5H,6H- | | 8.0 Hz, 1H), 7.72 (d, |
| isomer) | | | | pyrrolo[3,4-c]pyrrol-2-yl]-2- | | J = 8.4 Hz, 1H), 7.63 (t, |
| 94-48 | ++ | | | hydroxyethan-1-one; | | J = 7.6 Hz, 1H), 7.36 (t, |
| (2^{nd} | | | | (2R)-2-(1,2-benzoxazol-3-yl)-1- | | J = 7.6 Hz, 1H), 7.30- |
| eluting | | | | [5-(2,3-dihydro-1,4-benzodioxine- | | 7.20 (m, 2H), 7.04 (d, |
| isomer) | | | | 6-sulfonyl)-1H,2H,3H,4H,5H,6H- | | J = 8.4 Hz, 1H), 6.31 (d, |
| | | | | pyrrolo[3,4-c]pyrrol-2-yl]-2- | | J = 6.4 Hz, 1H), 5.71 |
| | | | | hydroxyethan-1-one | | (d, J = 6.4 Hz, 1H), |
| | | | | | | 4.44-4.42 (m, 1H), |
| | | | | | | 4.31-4.29 (m, 4H), |
| | | | | | | 4.16-3.94 (m, 7H). |
| 94-49 | + | 45-2, 46-1, | A | (2S)-1-[5-(2,3-dihydro-1,4- | 541.3 | (CDCl₃, 400 MHz) δ |
| (1^{st} | | 2-2 | | benzodioxine-6-sulfonyl)- | | (ppm): 7.53-7.40 (m, |
| eluting | | | | 1H,2H,3H,4H,5H,6H-pyrrolo[3,4- | | 3H), 7.34-7.27 (m, |
| isomer) | | | | c]pyrrol-2-yl]-2-[4-(4- | | 2H), 7.13-7.04 (m, |
| 94-50 | ++ | | | fluorophenyl)-1-methyl-1H- | | 2H), 6.97-6.95 (m, |
| (2^{nd} | | | | pyrazol-3-yl]-2-hydroxyethan-1- | | 1H), 5.10 (s, 1H), 4.38- |
| eluting | | | | one; | | 3.98 (m, 10H), 3.90 (s, |
| isomer) | | | | (2R)-1-[5-(2.3-dihydro-1,4- | | 3H), 3.86-3.71 (m, |
| | | | | benzodioxine-6-sulfonyl)- | | 1H), 3.44-3.41 (m, |
| | | | | 1H,2H,3H,4H,5H,6H-pyrrolo[3,4- | | 1H). |
| | | | | c]pyrrol-2-yl]-2-[4-(4- | | |
| | | | | fluorophenyl)-1-methyl-1H- | | |
| | | | | pyrazol-3-yl]-2-hydroxyethan-1- | | |
| | | | | one | | |
| 94-51 | +++ | 39-1, 51-1 | A | (2S)-1-(5-{2H,3H- | 497.0 | (CD₃OD, 400 MHz) |
| (1^{st} | | | | [1,4]dioxino[2,3-b]pyridine-7- | | (ppm): 8.14 (s, 1H), |
| eluting | | | | sulfonyl}-1H,2H,3H,4H,5H,6H- | | 7.63 (d, J = 2.4 Hz, |
| isomer) | | | | pyrrolo[3,4-c]pyrrol-2-yl)-2- | | 1H), 7.48 (d, J = 8.0 |
| 94-52 | + | | | hydroxy-2-(1-methyl-1H-indol-2- | | Hz, 1H), 7.40-7.32 (m, |
| (2^{nd} | | | | yl)ethan-1-one; | | 1H), 7.17-7.15 (m, |
| eluting | | | | (2R)-1-(5-{2H,3H- | | 1H), 7.02-6.99 (m, |
| isomer) | | | | [1,4]dioxino[2,3-b]pyridine-7- | | 1H), 6.34 (s, 1H), 5.53 |
| | | | | sulfonyl}-1H,2H,3H,4H,5H,6H- | | (s, 1H), 4.54-4.47 (m, |
| | | | | pyrrolo[3,4-c]pyrrol-2-yl)-2- | | 2H), 4.34-3.97 (m, |
| | | | | hydroxy-2-(1-methyl-1H-indol-2- | | 9H), 3.82 (s, 3H), 3.77- |
| | | | | yl)ethan-1-one | | 3.74 (m, 1H). |
| 94-53 | + | 47-1, 38-1 | A | (2S)-1-[5-(3,4-dihydro-2H-1- | 441.3 | (DMSO-d₆, 400 MHz) |
| | | | | benzopyran-7-sulfonyl)- | | δ (ppm): 7.34-7.27 (m, |
| | | | | 1H,2H,3H,4H,5H,6H-pyrrolo[3,4- | | 6H), 7.22-7.20 (m, |
| | | | | c]pyrrol-2-yl]-2-hydroxy-2- | | 1H), 7.07-7.06 (m, |
| | | | | phenylethan-1-one | | 1H), 5.63 (d, J = 5.6 |
| | | | | | | Hz, 1H), 5.15 (d, J = |
| | | | | | | 5.2 Hz, 1H), 4.19-4.06 |
| | | | | | | (m, 3H), 4.05-3.85 (m, |
| | | | | | | 7H), 2.80 (t, J = 6.4 Hz, |
| | | | | | | 2H), 1.92 (t, J = 5.2 Hz, |
| | | | | | | 2H). |
| 94-54 | ++ | 2-1 | A | (2S)-1-[5-(2,3-dihydro-1,4- | 441.4 | |
| | | | | benzodioxine-6-sulfonyl)- | | |
| | | | | 1H,2H,3H,5H-pyrrolo[3,4- | | |
| | | | | c]pyrrol-2-yl]-2-hydroxy-2- | | |
| | | | | phenylethan-1-one | | |
| 94-55 | ++ | 2-2 | A | 1-[5-(2,3-dihydro-1,4- | 427.2 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-phenylethan-1- | | |
| | | | | one | | |
| 94-56 | ++ | — | A | 2-(2,3-dihydro-1-benzofuran-5- | 484.1 | |
| | | | | yl)-1-(5-{2H,3H-[1,4]dioxino[2,3- | | |
| | | | | b]pyridine-7-sulfonyl}- | | |
| | | | | 1H,2H,3H,5H-pyrrolo[3,4- | | |
| | | | | c]pyrrol-2-yl)-2-hydroxyethan-1- | | |
| | | | | one | | |
| 95-1 | +++ | 2-3, 2-2 | B | (2R)-2-(3-cyclopropyl-4- | 540.5 | (DMSO-d₆, 300 MHz) |
| (1^{st} | | | | methoxyphenyl)-1-[5-(2,3- | | δ (ppm): 7.29-7.24 (m, |
| eluting | | | | dihydro-1,4-benzodioxine-6- | | 2H), 7.07-7.01 (m, |
| isomer) | | | | sulfonyl)-1H,2H,3H,4H,5H,6H- | | 2H), 6.99-6.84 (m, |
| 95-2 | +++ | | | pyrrolo[3,4-c]pyrrol-2-yl]-3- | | 1H), 6.72 (s, 1H), 4.31 |
| (2^{nd} | | | | (methylamino)propan-1-one; | | (s, 5H), 4.11-3.99 (m, |
| eluting | | | | (2S)-2-(3-cyclopropyl-4- | | 6H), 3.94-3.74 (m, |
| isomer) | | | | methoxyphenyl)-1-[5-(2,3- | | 5H), 3.11-3.04 (m, |
| | | | | dihydro-1,4-benzodioxine-6- | | 1H), 2.59-2.50 (m, |
| | | | | sulfonyl)-1H,2H,3H,4H,5H,6H- | | 1H), 2.28 (s, 3H), 2.08- |
| | | | | pyrrolo[3,4-c]pyrrol-2-yl]-3- | | 1.99 (m, 1H), 0.90-0.84 |
| | | | | (methylamino)propan-1-one | | (m, 2H), 0.58-0.54 (m, |
| | | | | | | 2H). |
| 95-3 | + | 3-1, 2-2 | B | (2R)-1-[5-(2,3-dihydro-1,4- | 500.5 | (CD₃OD, 400 MHz) δ |
| (1^{st} | | | | benzodioxine-6-sulfonyl)- | | (ppm): 7.32-7.24 (m, |
| eluting | | | | 1H,2H,3H,4H,5H,6H-pyrrolo[3,4- | | 3H), 7.07-6.99 (m, |
| isomer) | | | | c]pyrrol-2-yl]-2-(3- | | 1H), 6.88-6.85 (m, |
| 95-4 | ++++ | | | methoxyphenyl)-3- | | 3H), 4.38-4.31 (m, |
| (2^{nd} | | | | (methylamino)propan-1-one; | | 5H), 4.20-4.17 (m, |
| eluting | | | | (2S)-1-[5-(2,3-dihydro-1,4- | | 1H), 4.08-4.04 (m, |
| isomer) | | | | benzodioxine-6-sulfonyl)- | | 5H), 3.89-3.86 (m, |
| | | | | 1H,2H,3H,4H,5H,6H-pyrrolo[3,4- | | 2H), 3.78 (s, 3H), 3.32- |
| | | | | c]pyrrol-2-yl]-2-(3- | | 3.23 (m, 1H), 2.74-2.69 |
| | | | | methoxyphenyl)-3- | | (m, 1H), 2.39 (s, 3H). |
| | | | | (methylamino)propan-1-one | | |
| 95-5 | ++++ | 3-3, 10-1, | B | (2R)-1-[5-(2,3-dihydro-1,4- | 498.4 | (DMSO-d₆, 400 MHz) |
| | | 2-2 | | benzodioxine-6-sulfonyl)- | | δ (ppm): 7.28-7.18 (m, |
| 95-6 | ++ | | | 1H,2H,3H,4H,5H,6H-pyrrolo[3,4- | | 3H), 7.11-7.04 (m, |
| | | | | c]pyrrol-2-yl]-2-(3-ethylphenyl)-3- | | 4H), 4.03-4.30 (m, |
| | | | | (methylamino)propan-1-one; | | 5H), 4.06-3.80 (m, |
| | | | | (2S)-1-[5-(2,3-dihydro-1,4- | | 8H), 3.11-3.06 (m, |
| | | | | benzodioxine-6-sulfonyl)- | | 1H), 2.56-2.50 (m, |
| | | | | 1H,2H,3H,4H,5H,6H-pyrrolo[3,4- | | 4H), 2.32 (s, 3H), |
| | | | | c]pyrrol-2-yl]-2-(3-ethylphenyl)-3- | | 1.23-1.12 (m, 3H). |
| | | | | (methylamino)propan-1-one | | |
| 95-7 | ++ | 3-4, 12-1, | B | (2R)-2-(3-cyclopropylphenyl)-1- | 510.4 | (DMSO-d₆, 400 MHz) |
| | | 2-2 | | [5-(2,3-dihydro-1,4-benzodioxine- | | δ (ppm): 7.28-7.24 (m, |
| 95-8 | ++++ | | | 6-sulfonyl)-1H,2H,3H,4H,5H,6H- | | 2H), 7.17-7.13 (m, |
| | | | | pyrrolo[3,4-c]pyrrol-2-yl]-3- | | 1H), 7.06-7.01 (m, |
| | | | | (methylamino)propan-1-one; (2S)- | | 3H), 6.88-6.86 (d, J = |
| | | | | 2-(3-cyclopropylphenyl)-1-[5- | | 7.6 Hz, 1H), 4.38-4.28 |
| | | | | (2,3-dihydro-1,4-benzodioxine-6- | | (m, 5H), 4.05-3.86 (m, |
| | | | | sulfonyl)-1H,2H,3H,4H,5H,6H- | | 7H), 3.80-3.76 (m, |
| | | | | pyrrolo[3,4-c]pyrrol-2-yl]-3- | | 1H), 3.07-3.02 (m, |
| | | | | (methylamino)propan-1-one | | 1H), 2.54-2.50 (m, |
| | | | | | | 1H), 2.23 (s, 3H), 1.88- |
| | | | | | | 1.81 (m, 1H), 1.60 (s, |
| | | | | | | 1H), 0.94-0.89 (m, |
| | | | | | | 2H), 0.65-0.59 (m, |
| | | | | | | 2H). |
| 95-9 | ++ | 3-5, 8-1, | B | (2R)-2-(3-cyclopropoxyphenyl)-1- | 526.4 | (DMSO-d₆, 400 MHz) |
| | | 2-2 | | [5-(2,3-dihydro-1,4-benzodioxine- | | δ (ppm): 7.28-7.19 (m, |
| 95-10 | ++++ | | | 6-sulfonyl)-1H,2H,3H,4H,5H,6H- | | 3H), 7.06-7.04 (d, J = |
| | | | | pyrrolo[3,4-c]pyrrol-2-yl]-3- | | 8.4 Hz, 1H), 6.95-6.93 |
| | | | | (methylamino)propan-1-one; (2S)- | | (m, 1H), 6.91 (s, 1H), |
| | | | | 2-(3-cyclopropoxyphenyl)-1-[5- | | 6.87-6.85 (m, 1H), |
| | | | | (2,3-dihydro-1,4-benzodioxine-6- | | 4.38-4.30 (m, 5H), |
| | | | | sulfonyl)-1H,2H,3H,4H,5H,6H- | | 4.00-3.88 (m, 7H), |
| | | | | pyrrolo[3,4-c]pyrrol-2-yl]-3- | | 3.82-3.75 (m, 2H), |
| | | | | (methylamino)propan-1-one | | 3.07-3.02 (m, 1H), |
| | | | | | | 2.55-2.50 (m, 1H), 2.23 |
| | | | | | | (s, 3H), 1.52 (s, 1H), |
| | | | | | | 0.77-0.63 (m, 2H), |
| | | | | | | 0.65-0.61 (m, 2H). |
| 95-11 | + | 3-6, 11-1, | B | (2R)-2-(3,5-dichlorophenyl)-1-[5- | 538.3 | (CD₃OD, 400 MHz) |
| | | 2-2 | | (2,3-dihydro-1,4-benzodioxine-6- | | (ppm): 7.36-7.29 (m, |
| 95-12 | ++++ | | | sulfonyl)-1H,2H,3H,4H,5H,6H- | | 5H), 7.00-6.97 (d, J = |
| | | | | pyrrolo[3,4-c]pyrrol-2-yl]-3- | | 8.8 Hz, 1H), 4.40-4.36 |
| | | | | (methylamino)propan-1-one; (2S)- | | (m, 1H), 4.30-4.27 (m, |
| | | | | 2-(3,5-dichlorophenyl)-1-[5-(2,3- | | 5H), 4.18-4.06 (m, |
| | | | | dihydro-1,4-benzodioxine-6- | | 5H), 3.95-3.90 (m, |
| | | | | sulfonyl)-1H,2H,3H,4H,5H,6H- | | 2H), 3.21-3.15 (m, |
| | | | | pyrrolo[3,4-c]pyrrol-2-yl]-3- | | 1H), 2.74-2.70 (m, |
| | | | | (methylamino)propan-1-one | | 1H), 2.36 (s, 3H). |
| 95-13 | ++++ | 3-7, 13-1, | B | (2R)-2-(3-chloro-5- | 534.3 | (CD₃OD, 400 MHz) δ |
| | | 2-2 | | methoxyphenyl)-1-[5-(2,3- | | (ppm): 7.34-7.31 (m, |
| | | | | dihydro-1,4-benzodioxine-6- | | 2H), 7.02-7.00 (d, J = |
| | | | | sulfonyl)-1H,2H,3H,4H,5H,6H- | | 8.8 Hz, 1H), 6.94-6.90 |
| | | | | pyrrolo[3,4-c]pyrrol-2-yl]-3- | | (m, 1H), 6.89-6.86 (m, |
| | | | | (methylamino)propan-1-one; (2S)- | | 1H), 6.84-6.83 (m, |
| 95-14 | ++ | | | 2-(3-chloro-5-methoxyphenyl)-1- | | 1H), 4.37-4.29 (m, |
| | | | | [5-(2,3-dihydro-1,4-benzodioxine- | | 5H), 4.21-4.07 (m, |
| | | | | 6-sulfonyl)-1H,2H,3H,4H,5H,6H- | | 6H), 3.92-3.86 (m, |
| | | | | pyrrolo[3,4-c]pyrrol-2-yl]-3- | | 2H), 3.79 (s, 3H), 3.25- |
| | | | | (methylamino)propan-1-one | | 3.19 (m, 1H), 2.75-2.71 |
| | | | | | | (m, 1H), 2.40 (s, 3H). |
| 95-15 | ++++ | 3-8, 9-1, | B | (2R)-1-[5-(2,3-dihydro-1,4- | 521.2 | (DMSO-d₆, 300 MHz) |
| | | 2-2 | | benzodioxine-6-sulfonyl)- | | δ (ppm): 8.97-8.95 (m, |
| 95-16 | ++ | | | 1H,2H,3H,4H,5H,6H-pyrrolo[3,4- | | 1H), 8.40-8.36 (m, |
| | | | | c]pyrrol-2-yl]-3-(methylamino)-2- | | 1H), 7.90-7.87 (m, |
| | | | | (quinolin-8-yl)propan-1-one; (2S)- | | 1H), 7.69-7.67 (m, |
| | | | | 1-[5-(2,3-dihydro-1,4- | | 1H), 7.59-7.55 (m, |
| | | | | benzodioxine-6-sulfonyl)- | | 2H), 7.26-7.22 (m, |
| | | | | 1H,2H,3H,4H,5H,6H-pyrrolo[3,4- | | 2H), 7.04-7.02 (d, J = |
| | | | | c]pyrrol-2-yl]-3-(methylamino)-2- | | 8.4 Hz, 1H), 5.44-5.38 |
| | | | | (quinolin-8-yl)propan-1-one | | (m, 1H), 4.30-4.27 (m, |
| | | | | | | 5H), 4.13-3.92 (m, |
| | | | | | | 7H), 3.22-3.19 (m, |
| | | | | | | 1H), 2.71-2.63 (m, |
| | | | | | | 1H), 2.28 (s, 3H). |
| 95-17 | ++++ | 3-9, 2-2 | B | (2R)-1-[5-(2,3-dihydro-1,4- | 502.2 | (DMSO-d₆, 300 MHz) |
| 95-18 | ++ | | | benzodioxine-6-sulfonyl)- | | δ (ppm): 7.29-7.23 (m, |
| | | | | 1H,2H,3H,4H,5H,6H-pyrrolo[3,4- | | 2H), 7.12-7.03 (m, |
| | | | | c]pyrrol-2-yl]-2-(2-fluoro-5- | | 4H), 4.32-4.29 (m, |
| | | | | methylphenyl)-3- | | 5H), 4.10-3.96 (m, |
| | | | | (methylamino)propan-1-one; (2S)- | | 8H), 3.09-3.02 (m, |
| | | | | 1-[5-(2,3-dihydro-1,4- | | 1H), 2.59-2.57 (m, |
| | | | | benzodioxine-6-sulfonyl)- | | 1H), 2.23 (s, 6H). |
| | | | | 1H,2H,3H,4H,5H,6H-pyrrolo[3,4- | | |
| | | | | c]pyrrol-2-yl]-2-(2-fiuoro-5- | | |
| | | | | methylphenyl)-3- | | |
| | | | | (methylamino)propan-1-one | | |
| 95-19 | + | 3-10, 2-2 | B | (2S)-2-(5-chloro-2-fluorophenyl)- | 522.3 | (CD₃OD, 400 MHz) δ |
| | | | | 1-[5-(2,3-dihydro-1,4- | | (ppm): 7.36-7.29 (m, |
| | | | | benzodioxine-6-sulfonyl)- | | 4H), 7.16-7.11 (m, |
| | | | | 1H,2H,3H,4H,5H,6H-pyrrolo[3,4- | | 1H), 7.00-6.97 (m, |
| | | | | c]pyrrol-2-yl]-3- | | 1H), 4.42-4.39 (m, |
| | | | | (methylamino)propan-1-one | | 1H), 4.29-4.23 (m, |
| 95-20 | +++ | | | (2R)-2-(5-chloro-2-fluorophenyl)- | | 5H), 4.20-4.16 (m, |
| | | | | 1-[5-(2,3-dihydro-1,4- | | 1H), 4.07-4.05 (m, |
| | | | | benzodioxine-6-sulfonyl)- | | 5H), 3.92-3.88 (m, |
| | | | | 1H,2H,3H,4H,5H,6H-pyrrolo[3,4- | | 1H), 3.23-3.18 (m, |
| | | | | c]pyrrol-2-yl]-3- | | 1H), 2.72-2.68 (m, |
| | | | | (methylamino)propan-1-one | | 1H), 2.36 (s, 3H). |
| 95-21 | ++ | 3-11, 2-2 | B | (2R)-2-(3-chloro-5-fluorophenyl)- | 522.2 | (CD₃OD, 400 MHz) δ |
| | | | | 1-[5-(2,3-dihydro-1,4- | | (ppm): 7.32-7.29 (m, |
| | | | | benzodioxine-6-sulfonyl)- | | 2H), 7.21 (s, 1H), 7.14- |
| | | | | 1H,2H,3H,4H,5H,6H-pyrrolo[3,4- | | 7.08 (m, 2H), 7.05-6.96 |
| | | | | c]pyrrol-2-yl]-3- | | (m, 1H), 4.40-4.36 (m, |
| | | | | (methylamino)propan-1-one; (2S)- | | 1H), 4.28 (s, 4H), 4.18- |
| 95-22 | ++++ | | | 2-(3-chloro-5-fluorophenyl)-1-[5- | | 4.14 (m, 1H), 4.06-3.94 |
| | | | | (2,3-dihydro-1,4-benzodioxine-6- | | (m, 5H), 3.93-3.91 |
| | | | | sulfonyl)-1H,2H,3H,4H,5H,6H- | | (m, 2H), 3.21-3.18 (m, |
| | | | | pyrrolo[3,4-c]pyrrol-2-yl]-3- | | 1H), 2.73-2.70 (m, |
| | | | | (methylamino)propan-1-one | | 1H), 2.36 (s, 3H). |
| 95-23 | + | 3-12, 2-2 | B | (2R)-2-(3-chloro-4-fluorophenyl)- | 522.1 | (CD₃OD, 400 MHz) δ |
| 95-24 | +++ | | | 1-[5-(2,3-dihydro-1,4- | | (ppm): 7.50-7.47 (m, |
| | | | | benzodioxine-6-sulfonyl)- | | 1H), 7.34-7.30 (m, |
| | | | | 1H,2H,3H,4H,5H,6H-pyrrolo[3,4- | | 3H), 7.29-7.21 (m, |
| | | | | c]pyrrol-2-yl]-3- | | 1H), 7.02-7.00 (m, |
| | | | | (methylamino)propan-1-one; (2S)- | | 1H), 4.41-4.30 (m, |
| | | | | 2-(3-chloro-4-fluorophenyl)-1-[5- | | 5H), 4.22-4.06 (m, |
| | | | | (2,3-dihydro-1,4-benzodioxine-6- | | 6H), 3.97-3.89 (m, |
| | | | | sulfonyl)-1H,2H,3H,4H,5H,6H- | | 2H), 3.32-3.21 (m, |
| | | | | pyrrolo[3,4-c]pyrrol-2-yl]-3- | | 1H), 2.79-2.74 (m, |
| | | | | (methylamino)propan-1-one | | 1H), 2.40 (s, 3H). |
| 95-25 | ++ | 3-13, 2-2 | B | (2R)-2-(3-chloro-2-fluorophenyl)- | 522.1 | (CD₃OD, 300 MHz) δ |
| 95-26 | ++++ | | | 1-[5-(2,3-dihydro-1,4- | | (ppm): 7.45-7.39 (m, |
| | | | | benzodioxine-6-sulfonyl)- | | 1H), 7.31-7.29 (m, |
| | | | | 1H,2H,3H,4H,5H,6H-pyrrolo[3,4- | | 3H), 7.18-7.13 (m, |
| | | | | c]pyrrol-2-yl]-3- | | 1H), 7.00-6.97 (d, J = |
| | | | | (methylamino)propan-1-one; (2S)- | | 9.0 Hz, 1H), 4.31-4.28 |
| | | | | 2-(3-chloro-2-fluorophenyl)-1-[5- | | (m, 7H), 4.08-4.04 (m, |
| | | | | (2,3-dihydro-1,4-benzodioxine-6- | | 5H), 3.83-3.78 (m, |
| | | | | sulfonyl)-1H,2H,3H,4H,5H,6H- | | 1H), 3.30-3.29 (m, |
| | | | | pyrrolo[3,4-c]pyrrol-2-yl]-3- | | 1H), 2.82-2.78 (m, |
| | | | | (methylamino)propan-1-one | | 1H), 2.42 (s, 3H). |
| 95-27 | ++ | 3-14, 2-2 | B | (2R)-2-(5-chloro-2- | 534.2 | (DMSO-d₆, 400 MHz) |
| 95-28 | ++++ | | | methoxyphenyl)-1-[5-(2,3- | | δ (ppm): 7.29-7.24 (m, |
| | | | | dihydro-1,4-benzodioxine-6- | | 3H), 7.18-7.17 (d, J = |
| | | | | sulfonyl)-1H,2H,3H,4H,5H,6H- | | 3.2 Hz, 1H), 7.07-7.00 |
| | | | | pyrrolo[3,4-c]pyrrol-2-yl]-3- | | (m, 2H), 4.36-4.29 (m, |
| | | | | (methylamino)propan-1-one; (2S)- | | 5H), 4.24-4.20 (m, |
| | | | | 2-(5-chloro-2-methoxyphenyl)-1- | | 1H), 4.01-3.90 (m, |
| | | | | [5-(2,3-dihydro-1,4-benzodioxine- | | 7H), 3.80-3.77 (s, 3H), |
| | | | | 6-sulfonyl)-1H,2H,3H,4H,5H,6H- | | 3.01-2.96 (m, 1H), |
| | | | | pyrrolo[3,4-c]pyrrol-2-yl]-3- | | 2.53-2.50 (m, 1H), |
| | | | | (methylamino)propan-1-one | | 2.22 (s, 3H), 1.66 (s, |
| | | | | | | 1H). |
| 95-29 | ++++ | 2-8, 2-2, | B | (2R)-2-(3-chloro-4- | 560.4 | (DMSO-d₆, 400 MHz) |
| (1^{st} | | 3-16, 5-2 | | cyclopropoxyphenyl)-1-[5-(2,3- | | δ (ppm): 7.41-7.35 (m, |
| eluting | | | | dihydro-1,4-benzodioxine-6- | | 2H), 7.29-7.22 (m, |
| isomer) | | | | sulfonyl)-1H,2H,3H,4H,5H,6H- | | 3H), 7.06 (d, J = 8.4 |
| 95-30 | ++ | | | pyrrolo[3,4-c]pyrrol-2-yl]-3- | | Hz, 1H), 4.33-4.29 (m, |
| (2^{nd} | | | | (methylamino)propan-1-one; | | 5H), 4.08-3.76 (m, |
| eluting | | | | (2S)-2-(3-chloro-4- | | 9H), 3.35 (d, J = 12.0 |
| isomer) | | | | cyclopropoxyphenyl)-1-[5-(2,3- | | Hz, 1H), 2.91-3.12 (m, |
| | | | | dihydro-1,4-benzodioxine-6- | | 1H), 2.51-2.48 (m, |
| | | | | sulfonyl)-1H,2H,3H,4H,5H,6H- | | 3H), 1.23 (s, 1H), 0.82 |
| | | | | pyrrolo[3,4-c]pyrrol-2-yl]-3- | | (d, J = 6.8 Hz, 2H), |
| | | | | (methylamino)propan-1-one | | 0.68 (s, 2H). |
| 95-31 | ++++ | 2-9, 14-1, | B | (2R)-2-(3-chloro-4-fluoro-2- | 552.4 | (CD₃OD, 400 MHz) δ |
| (1^{st} | | 2-2 | | methoxyphenyl)-1-[5-(2,3- | | (ppm): 7.30-7.24 (m, |
| eluting | | | | dihydro-1,4-benzodioxine-6- | | 3H), 6.99-6.97 (m, |
| isomer) | | | | sulfonyl)-1H,2H,3H,4H,5H,6H- | | 2H), 4.36-4.28 (m, |
| 95-32 | ++ | | | pyrrolo[3,4-c]pyrrol-2-yl]-3- | | 6H), 4.27-4.18 (m, |
| (2^{nd} | | | | (methylamino)propan-1-one; | | 1H), 4.15-4.04 (m, |
| eluting | | | | (2S)-2-(3-chloro-4-fluoro-2- | | 5H), 3.87 (s, 3H), 3.82- |
| isomer) | | | | methoxyphenyl)-1-[5-(2,3- | | 3.78 (m, 1H), 3.20-3.14 |
| | | | | dihydro-1,4-benzodioxine-6- | | (m, 1H), 2.71-2.67 (m, |
| | | | | sulfonyl)-1H,2H,3H,4H,5H,6H- | | 1H), 2.43 (s, 3H). |
| | | | | pyrrolo[3,4-c]pyrrol-2-yl]-3- | | |
| | | | | (methylamino)propan-1-one | | |
| 95-33 | ++++ | 2-17, 5-3, | B | (2R)-2-(3-chloro-4-fluoro-5- | 552.2 | (CD₃OD, 400 MHz) δ |
| (1^{st} | | 2-2 | | methoxyphenyl)-1-[5-(2,3- | | (ppm): 7.31-7.28 (m, |
| eluting | | | | dihydro-1,4-benzodioxine-6- | | 2H), 7.06-6.97 (m, |
| isomer) | | | | sulfonyl)-1H,2H,3H,4H,5H,6H- | | 3H), 4.39-4.35 (m, |
| 95-34 | + | | | pyrrolo[3,4-c]pyrrol-2-yl]-3- | | 1H), 4.30-4.27 (m, |
| (2^{nd} | | | | (methylamino)propan-1-one; | | 4H), 4.19-4.15 (m, |
| eluting | | | | (2S)-2-(3-chloro-4-fluoro-5- | | 1H), 4.07-4.05 (m, |
| isomer) | | | | methoxyphenyl)-1-[5-(2,3- | | 5H), 3.95-3.85 (m, |
| | | | | dihydro-1,4-benzodioxine-6- | | 5H), 3.22-3.17 (m, |
| | | | | sulfonyl)-1H,2H,3H,4H,5H,6H- | | 1H), 2.75-2.71 (m, |
| | | | | pyrrolo[3,4-c]pyrrol-2-yl]-3- | | 1H), 2.38 (s, 3H). |
| | | | | (methylamino)propan-1-one | | |
| 95-35 | ++++ | 13-3, 17-1, | B | (2R)-1-[5-(2,3-dihydro-1,4- | 532.5 | (DMSO-d₆, 400 MHz) |
| (1^{st} | | 18-1, 2-2 | | benzodioxine-6-sulfonyl)- | | δ (ppm): 7.29-7.24 (m, |
| eluting | | | | 1H,2H,3H,4H,5H,6H-pyrrolo[3,4- | | 2H), 7.08-7.02 (m, |
| isomer) | | | | c]pyrrol-2-yl]-2-(4-fluoro-2- | | 2H), 6.84 (d, J = 11.6 |
| 95-36 | + | | | methoxy-5-methylphenyl)-3- | | Hz, 1H), 4.32-4.31 (m, |
| (2^{nd} | | | | (methylamino)propan-1-one; | | 5H), 4.17-4.13 (m, |
| eluting | | | | (2S)-1-[5-(2,3-dihydro-1,4- | | 1H), 4.01-3.92 (m, |
| isomer) | | | | benzodioxine-6-sulfonyl)- | | 6H), 3.86-3.78 (m, |
| | | | | 1H,2H,3H,4H,5H,6H-pyrrolo[3,4- | | 4H), 3.01 (t, J = 11.2 |
| | | | | c]pyrrol-2-yl]-2-(4-fluoro-2- | | Hz, 1H), 2.47-2.43 (m, |
| | | | | methoxy-5-methylphenyl)-3- | | 1H), 2.23 (s, 3H), 2.10 |
| | | | | (methylamino)propan-1-one | | (s, 3H), 1.51 (br s, 1H). |
| 95-37 | ++++ | 2-6, 17-2, | B | (2R)-1-[5-(2,3-dihydro-1,4- | 532.4 | (DMSO-d₆, 300 MHz) |
| (1^{st} | | 2-2 | | benzodioxine-6-sulfonyl)- | | δ (ppm): 7.29-7.24 (m, |
| eluting | | | | 1H,2H,3H,4H,5H,6H-pyrrolo[3,4- | | 2H), 7.08-7.03 (m, |
| isomer) | | | | c]pyrrol-2-yl]-2-(2-fluoro-4- | | 2H), 6.80 (d, J = 12.3 |
| 95-38 | + | | | methoxy-5-methylphenyl)-3- | | Hz, 1H), 4.40-4.01 (m, |
| (2^{nd} | | | | (methylamino)propan-1-one; | | 5H), 3.99-3.91 (m, |
| eluting | | | | (2S)-1-[5-(2,3-dihydro-1,4- | | 7H), 3.86-3.82 (m, |
| isomer) | | | | benzodioxine-6-sulfonyl)- | | 1H), 3.76 (s, 3H), 3.08- |
| | | | | 1H,2H,3H,4H,5H,6H-pyrrolo[3,4- | | 3.02 (m, 1H), 2.57-2.51 |
| | | | | c]pyrrol-2-yl]-2-(2-fluoro-4- | | (m, 1H), 2.24 (s, 3H), |
| | | | | methoxy-5-methylphenyl)-3- | | 2.05-2.00 (m, 3H), |
| | | | | (methylamino)propan-1-one | | 1.91-1.63 (m, 1H). |
| 95-39 | ++++ | 2-7, 3-15, | B | (2R)-2-(5-chloro-2-methoxy-4- | 548.4 | (CD₃OD, 300 MHz) δ |
| (1^{st} | | 2-2, 5-1 | | methylphenyl)-1-[5-(2,3-dihydro- | | (ppm): 7.33-7.29 (m, |
| eluting | | | | 1,4-benzodioxine-6-sulfonyl)- | | 2H), 7.14 (s, 1H), 7.00- |
| isomer) | | | | 1H,2H,3H,4H,5H,6H-pyrrolo[3,4- | | 6.96 (m, 2H), 4.38-4.29 |
| 95-40 | ++ | | | c]pyrrol-2-yl]-3- | | (m, 6H), 4.21-4.05 |
| (2^{nd} | | | | (methylamino)propan-1-one; | | (m, 6H), 3.88-3.77 (m, |
| eluting | | | | (2S)-2-(5-chloro-2-methoxy-4- | | 4H), 3.33-3.13 (m, |
| isomer) | | | | methylphenyl)-1-[5-(2,3-dihydro- | | 1H), 2.66-2.62 (m, |
| | | | | 1,4-benzodioxine-6-sulfonyl)- | | 1H), 2.40 (s, 3H), 2.34 |
| | | | | 1H,2H,3H,4H,5H,6H-pyrrolo[3,4- | | (s, 3H). |
| | | | | c]pyrrol-2-yl]-3- | | |
| | | | | (methylamino)propan-1-one | | |
| 95-41 | ++++ | | | (2R)-1-[5-(2,3-dihydro-1,4- | | (DMSO-d₆, 400 MHz) |
| (1^{st} | | | | benzodioxine-6-sulfonyl)- | | δ (ppm): 7.28-7.23 (m, |
| eluting | | | | 1H,2H,3H,4H,5H,6H-pyrrolo[3,4- | | 2H), 7.05 (d, J = 11.2 |
| isomer) | | | | c]pyrrol-2-yl]-2-(4-methoxy-2,5- | | Hz, 1H), 6.87 (s, 1H), |
| 95-42 | ++ | 2-23, 2-2 | B | dimethylphenyl)-3- | 528.5 | 6.75 (s, 1H), 4.36-4.30 |
| (2^{nd} | | | | (methylamino)propan-1-one; | | (s, 5H), 4.02-3.97 (m, |
| eluting | | | | (2S)-1-[5-(2,3-dihydro-1,4- | | 6H), 3.87-3.82 (m, |
| isomer) | | | | benzodioxine-6-sulfonyl)- | | 1H), 3.53-3.48 (m, |
| | | | | 1H,2H,3H,4H,5H,6H-pyrrolo[3,4- | | 1H), 3.02-2.95 (m, |
| | | | | c]pyrro1-2-yl]-2-(4-methoxy-2,5- | | 1H), 2.61-2.32 (m, |
| | | | | dimethylphenyl)-3- | | 4H), 2.24 (s, 3H), 2.11 |
| | | | | (methylamino)propan-1-one | | (s, 3H). |
| 95-43 | ++++ | | B | (2R)-1-[5-(2,3-dihydro-1,4- | 516.4 | (CD₃OD, 300 MHz) δ |
| (1^{st} | | | | benzodioxine-6-sulfonyl)- | | (ppm): 7.30-7.26 (m, |
| eluting | | | | 1H,2H,3H,4H,5H,6H-pyrrolo[3,4- | | 2H), 7.00-6.89 (m, |
| isomer) | | | | c]pyrrol-2-yl]-2-(4-fluoro-2,5- | | 3H), 4.33-4.27 (m, |
| 95-44 | ++ | 2-16, 2-2 | | dimethylphenyl)-3- | | 5H), 4.27-3.99 (m, |
| (2^{nd} | | | | (methylamino)propan-1-one; | | 7H), 3.55-3.50 (m, |
| eluting | | | | (2S)-1-[5-(2,3-dihydro-1,4- | | 1H), 3.20-3.16 (m, |
| isomer) | | | | benzodioxine-6-sulfonyl)- | | 1H), 2.61-2.56 (m, |
| | | | | 1H,2H,3H,4H,5H,6H-pyrrolo[3,4- | | 1H), 2.47 (s, 3H), 2.39 |
| | | | | c]pyrrol-2-yl]-2-(4-fluoro-2,5- | | (s, 3H), 2.17 (s, 3H). |
| | | | | dimethylphenyl)-3- | | |
| | | | | (methylamino)propan-1-one | | |
| 95-45 | +++ | 28-1, 2-2, | B | (2S)-2-(2-cyclopropyl-1,3-thiazol- | 517.4 | (CDCl₃, 400 MHz) δ |
| | | 29-1 | | 4-yl)-1-[5-(2,3-dihydro-1,4- | | (ppm): 7.55-7.34 (m, |
| | | | | benzodioxine-6-sulfonyl)- | | 1H), 7.29 (s, 1H), 7.01 |
| | | | | 1H,2H,3H,4H,5H,6H-pyrrolo[3,4- | | (d, J = 8.4 Hz, 1H), |
| | | | | c]pyrrol-2-yl]-3- | | 6.90 (s, 1H), 4.34-4.33 |
| | | | | (methylamino)propan-1-one; or | | (m, 5H), 4.20-4.00 (m, |
| | | | | (2R)-2-(2-cyclopropyl-1,3-thiazol- | | 9H), 3.33-3.30 (m, |
| | | | | 4-yl)-1-[5-(2,3-dihydro-1,4- | | 1H), 2.91-2.86 (m, |
| | | | | benzodioxine-6-sulfonyl)- | | 1H), 2.46 (s, 3H), 2.34- |
| | | | | 1H,2H,3H,4H,5H,6H-pyrrolo[3,4- | | 2.27 (m, 1H), 1.17-1.11 |
| | | | | c]pyrrol-2-yl]-3- | | (m, 2H), 1.01-0.97 (m, |
| | | | | (methylamino)propan-1-one | | 2H). |
| 95-46 | ++++ | 28-1, 2-2, | B | (2R)-2-(3-cyclobutoxy-4- | 568.5 | (CDCl₃, 300 MHz) δ |
| | | 29-1 | | methylphenyl)-1-[5-(2,3-dihydro- | | (ppm): 7.36-7.28 (m, |
| | | | | 1,4-benzodioxine-6-sulfonyl)- | | 2H), 7.06-7.04 (m, |
| | | | | 1H,2H,3H,4H,5H,6H-pyrrolo[3,4- | | 1H), 6.99-6.96 (m, |
| | | | | c]pyrrol-2-yl]-4- | | 1H), 6.72-6.70 (m, |
| | | | | (methylamino)butan-1-one; or | | 1H), 6.65-6.63 (m, |
| | | | | (2S)-2-(3-cyclobutoxy-4- | | 1H), 4.63-4.62 (m, |
| | | | | methylphenyl)-1-[5-(2,3-dihydro- | | 1H), 4.33-4.25 (m, |
| | | | | 1,4-benzodioxine-6-sulfonyl)- | | 5H), 4.25-4.15 (m, |
| | | | | 1H,2H,3H,4H,5H,6H-pyrrolo[3,4- | | 1H), 4.09-3.95 (m, |
| | | | | c]pyrrol-2-yl]-4- | | 5H), 3.93-3.88 (m, |
| | | | | (methylamino)butan-1-one | | 1H), 3.72-3.70 (m, |
| | | | | | | 1H), 2.64-2.55 (m, |
| | | | | | | 2H), 2.50-2.38 (m, |
| | | | | | | 5H), 2.29-2.22 (m, |
| | | | | | | 1H), 2.19 (s, 3H), 2.14- |
| | | | | | | 2.05 (m, 2H), 1.95-1.79 |
| | | | | | | (m, 3H), 1.75-1.65 (m, |
| | | | | | | 1H). |
| 95-47 | ++++ | 2-20, 2-2 | B | (2R)-2-(2,1,3-benzothiadiazol-4- | 528.4 | (DMSO-d₆, 400 MHz) |
| (1^{st} | | | | yl)-1-[5-(2,3-dihydro-1,4- | | δ (ppm): 7.98-7.96 (m, |
| eluting | | | | benzodioxine-6-sulfonyl)- | | 1H), 7.70-7.68 (m, |
| isomer) | | | | 1H,2H,3H,4H,5H,6H-pyrrolo[3,4- | | 1H), 7.58-7.57 (m, |
| 95-48 | ++ | | | c]pyrrol-2-yl]-3- | | 1H), 7.28-7.22 (m, |
| (2^{nd} | | | | (methylamino)propan-1-one; | | 2H), 7.04 (d, J = 8.4 |
| eluting | | | | (2S)-2-(2,1,3-benzothiadiazol-4- | | Hz, 1H), 4.83-4.81 (m, |
| isomer) | | | | yl)-1-[5-(2,3-dihydro-1,4- | | 1H), 4.48-4.45 (m, |
| | | | | benzodioxine-6-sulfonyl)- | | 1H), 4.31-4.28 (m, |
| | | | | 1H,2H,3H,4H,5H,6H-pyrrolo[3,4- | | 4H), 4.08-3.94 (m, |
| | | | | c]pyrrol-2-yl]-3- | | 7H), 3.20-3.18 (m, |
| | | | | (methylamino)propan-1-one | | 1H), 2.82-2.80 (m, |
| | | | | | | 1H), 2.28 (s, 3H). |
| 95-49 | +++ | 22-4, 2-2 | B | (2S)-1-[5-(2,3-dihydro-1,4- | 510.4 | (DMSO-d₆, 400 MHz) |
| (1^{st} | | | | benzodioxine-6-sulfonyl)- | | δ (ppm): 8.01 (s, 1H), |
| eluting | | | | 1H,2H,3H,4H,5H,6H-pyrrolo[3,4- | | 7.64-7.62 (m, 1H), |
| isomer) | | | | c]pyrrol-2-yl]-3-(methylamino)-2- | | 7.31-7.26 (m, 2H), |
| 95-50 | ++ | | | {pyrazolo[1,5-a]pyridin-7- | | 7.22-7.18 (m, 1H), |
| (2^{nd} | | | | yl}propan-1-one; | | 7.09-7.07 (m, 1H), |
| eluting | | | | (2R)-1-[5-(2,3-dihydro-1,4- | | 6.83-6.81 (m, 1H), |
| isomer) | | | | benzodioxine-6-sulfonyl)- | | 6.66-6.65 (m, 1H), |
| | | | | 1H,2H,3H,4H,5H,6H-pyrrolo[3,4- | | 4.89-4.85 (m, 1H), |
| | | | | c]pyrrol-2-yl]-3-(methylamino)-2- | | 4.50-4.37 (m, 1H), |
| | | | | {pyrazolo[1,5-a]pyridin-7- | | 4.34-4.31 (m, 5H), |
| | | | | yl}propan-1-one | | 4.04-3.95 (m, 6H), |
| | | | | | | 3.18-3.13 (m, 1H), |
| | | | | | | 2.97-2.93 (m, 1H), 2.33 |
| | | | | | | (s, 3H), 2.29-2.01 (m, |
| | | | | | | 1H). |
| 95-51 | ++++ | 2-14, 2-2, | B | (2R)-1-[5-(2,3-dihydro-1,4- | 542.4 | (DMSO-d₆, 400 MHz) |
| (1^{st} | | 24-1 | | benzodioxine-6-sulfonyl)- | | δ (ppm): 7.46 (s, 2H), |
| eluting | | | | 1H,2H,3H,4H,5H,6H-pyrrolo[3,4- | | 7.27-7.22 (m, 2H), |
| isomer) | | | | c]pyrrol-2-yl]-2-(7-methyl-2,1,3- | | 7.05-7.02 (m, 1H), |
| | | | | benzothiadiazol-4-yl)-3- | | 4.75-4.73 (m, 1H), |
| | | | | (methylamino)propan-1-one; | | 4.46-4.41 (m, 1H), |
| 95-52 | ++ | | | (2S)-1-[5-(2,3-dihydro-1,4- | | 4.31-4.29 (m, 4H), |
| (2^{nd} | | | | benzodioxine-6-sulfonyl)- | | 4.08-3.92 (m, 7H), |
| eluting | | | | 1H,2H,3H,4H,5H,6H-pyrrolo[3,4- | | 3.18-3.15 (m, 1H), |
| isomer) | | | | c]pyrrol-2-yl]-2-(7-methyl-2,1,3- | | 2.77-2.74 (m, 1H), 2.62 |
| | | | | benzothiadiazol-4-yl)-3- | | (s, 3H), 2.27 (s, 3H). |
| | | | | (methylamino)propan-1-one | | |
| 95-53 | ++++ | | | (2R)-1-[5-(2,3-dihydro-1,4- | | (DMSO-d₆, 400 MHz) |
| (1^{st} | | | | benzodioxine-6-sulfonyl)- | | δ (ppm): 8.33-8.31 (m, |
| eluting | | | | 1H,2H,3H,4H,5H,6H-pyrrolo[3,4- | | 1H), 7.68 (s, 1H), 7.29- |
| isomer) | | | | c]pyrrol-2-yl]-3-(methylamino)-2- | | 7.24 (m, 2H), 7.07-7.01 |
| 95-54 | + | 22-1, 2-2 | B | {2-methylimidazo[1,2-a]pyridin- | 524.4 | (m, 2H), 6.78-6.74 (m, |
| (2^{nd} | | | | 8-yl}propan-1-one; | | 1H), 4.65-4.61 (m, |
| eluting | | | | (2S)-1-[5-(2,3-dihydro-1,4- | | 1H), 4.48-4.41 (m, |
| isomer) | | | | benzodioxine-6-sulfonyl)- | | 1H), 4.33-4.29 (m, |
| | | | | 1H,2H,3H,4H,5H,6H-pyrrolo[3,4- | | 5H), 4.01-3.97 (m, |
| | | | | c]pyrrol-2-yl]-3-(methylamino)-2- | | 6H), 3.14-3.09 (m, |
| | | | | {2-methylimidazo[1,2-a]pyridin- | | 1H), 2.72-2.68 (m, |
| | | | | 8-yl}propan-1-one | | 1H), 2.32 (s, 3H), 2.26 |
| | | | | | | (s, 3H). |
| 95-55 | ++ | 2-2, 2-26 | B | 4-[(2R)-1-[5-(2,3-dihydro-1,4- | 598.4 | (CD₃OD, 400 MHz) δ |
| (1^{st} | | | | benzodioxine-6-sulfonyl)- | | (ppm): 8.04-8.02 (m, |
| eluting | | | | 1H,2H,3H,4H,5H,6H-pyrrolo[3,4- | | 1H), 7.57-7.54 (m, |
| isomer) | | | | c]pyrrol-2-yl]-3-(methylamino)-1- | | 1H), 7.47-7.44 (m, |
| 95-56 | + | | | oxopropan-2-yl]-N,N-dimethyl- | | 1H), 7.40-7.38 (m, |
| (2^{nd} | | | | 1,3-benzothiazole-2-carboxamide; | | 2H), 7.06-7.03 (m, |
| eluting | | | | 4-[(2S)-1-[5-(2,3-dihydro-1,4- | | 1H), 5.19-5.16 (m, |
| isomer) | | | | benzodioxine-6-sulfonyl)- | | 1H), 5.08-4.97 (m, |
| | | | | 1H,2H,3H,4H,5H,6H-pyrrolo[3,4- | | 2H), 4.40-4.30 (m, |
| | | | | c]pyrrol-2-yl]-3-(methylamino)-1- | | 6H), 4.23 (s, 4H), 3.35- |
| | | | | oxopropan-2-yl]-N,N-dimethyl- | | 3.32 (m, 1H), 2.97 (s, |
| | | | | 1,3-benzothiazole-2-carboxamide | | 3H), 2.86-2.77 (m, |
| | | | | | | 4H), 2.41 (s, 3H). |
| 95-57 | ++ | 22-2, 25-1, | B | 1-[5-(2,3-dihydro-1,4- | 525.5 | (CDCl₃, 400 MHz) δ |
| | | 2-2 | | benzodioxine-6-sulfonyl)- | | (ppm): 7.59-7.57 (m, |
| | | | | 1H,2H,3H,4H,5H,6H-pyrrolo[3,4- | | 1H), 7.47-7.43 (m, |
| | | | | c]pyrrol-2-yl]-2-{2-methyl- | | 1H), 7.36-7.34 (m, |
| | | | | [1,2,4]triazolo[1,5-a]pyridin-5- | | 2H), 7.04-6.98 (m, |
| | | | | yl}-3-(methylamino)propan-1-one | | 2H), 5.06-5.04 (m, 1H), |
| | | | | | | 4.46-4.43 (m, 1H), |
| | | | | | | 4.36-4.28 (m, 5H), |
| | | | | | | 4.18-4.01 (m, 6H), |
| | | | | | | 3.43-3.42 (m, 1H), |
| | | | | | | 3.04-3.01 (m, 1H), |
| | | | | | | 2.62-2.59 (s, 3H), 2.51, |
| | | | | | | 2.48 (s, 3H). |
| 95-58 | ++++ | 2-18, 2-2 | B | (2R)-2-(5-chloro-4-fluoro-2- | 552.4 | (CD₃OD, 400 MHz) δ |
| (1^{st} | | | | methoxyphenyl)-1-[5-(2,3- | | (ppm): 7.30-7.27 (m, |
| eluting | | | | dihydro-1,4-benzodioxine-6- | | 2H), 7.24-7.22 (m, |
| isomer) | | | | sulfonyl)-1H,2H,3H,4H,5H,6H- | | 1H), 7.05-6.97 (m, |
| | | | | | | 2H), 4.41-4.38 (m, |
| 95-59 | ++ | | | pyrrolo[3,4-c]pyrrol-2-yl]-3- | | 1H), 4.30-4.18 (m, |
| (2^{nd} | | | | (methylamino)propan-1-one; | | 6H), 4.10-4.02 (m, |
| eluting | | | | (2S)-2-(5-chloro-4-fluoro-2- | | 5H), 3.89 (s, 3H), 3.72- |
| isomer) | | | | methoxyphenyl)-1-[5-(2,3- | | 3.65 (m, 1H), 3.37-3.29 |
| | | | | dihydro-1,4-benzodioxine-6- | | (m, 1H), 2.97-2.93 (m, |
| | | | | sulfonyl)-1H,2H,3H,4H,5H,6H- | | 1H), 2.56 (s, 3H). |
| | | | | pyrrolo[3,4-c]pyrrol-2-yl]-3- | | |
| | | | | (methylamino)propan-1-one | | |
| 95-60 | ++++ | 2-21, 2-2, | B | (2R)-1-[5-(2,3-dihydro-1,4- | 525.0 | (DMSO-d₆, 400 MHz) |
| (1^{st} | | 31-1 | | benzodioxine-6-sulfonyl)- | | δ (ppm): 7.51 (d, J = |
| eluting | | | | 1H,2H,3H,4H,5H,6H-pyrrolo[3,4- | | 8.0 Hz, 1H), 7.29-7.21 |
| isomer) | | | | c]pyrrol-2-yl]-2-(2-methyl-1,3- | | (m, 4H), 7.05 (d, J = |
| 95-61 | ++ | | | benzoxazol-4-yl)-3- | | 8.4 Hz, 1H), 4.54-4.50 |
| (2^{nd} | | | | (methylamino)propan-1-one; | | (m, 1H), 4.43-4.40 (m, |
| eluting | | | | (2S)-1-[5-(2,3-dihydro-1,4- | | 1H), 4.31 (d, J = 5.2 |
| isomer) | | | | benzodioxine-6-sulfonyl)- | | Hz, 4H), 4.08-3.95 (m, |
| | | | | 1H,2H,3H,4H,5H,6H-pyrrolo[3,4- | | 7H), 3.20-3.14 (m, |
| | | | | c]pyrrol-2-yl]-2-(2-methyl-1,3- | | 1H), 2.68-2.65 (m, |
| | | | | benzoxazol-4-yl)-3- | | 4H), 2.26 (s, 3H), 1.80 |
| | | | | (methylamino)propan-1-one | | (S, 1H). |
| 95-62 | ++++ | 2-24, 2-2, | B | (2R)-1-[5-(2,3-dihydro-1,4- | 559.0 | (CDCl₃, 400 MHz) δ |
| (1^{st} | | 32-1 | | benzodioxine-6-sulfonyl)- | | (ppm): 7.46-7.42 (m, |
| eluting | | | | 1H,2H,3H,4H,5H,6H-pyrrolo[3,4- | | 1H), 7.34-7.28 (m, |
| isomer) | | | | c]pyrrol-2-yl]-2-(7-fluoro-2- | | 2H), 7.08-7.03 (m, |
| 95-63 | ++ | | | methyl-1,3-benzothiazol-4-yl)-3- | | 1H), 6.99-6.96 (m, |
| (2^{nd} | | | | (methylamino)propan-1-one; | | 1H), 5.04-5.00 (m, |
| eluting | | | | (2S)-1-[5-(2,3-dihydro-1,4- | | 1H), 4.38-4.31 (m, |
| isomer) | | | | benzodioxine-6-sulfonyl)- | | 6H), 4.13-4.04 (m, |
| | | | | 1H,2H,3H,4H,5H,6H-pyrrolo[3,4- | | 4H), 4.00-3.98 (m, |
| | | | | c]pyrrol-2-yl]-2-(7-fluoro-2- | | 1H), 3.82-3.77 (m, |
| | | | | methyl-1,3-benzothiazol-4-yl)-3- | | 1H), 3.41-3.38 (m, |
| | | | | (methylamino)propan-1-one | | 1H), 2.92-2.85 (m, |
| | | | | | | 4H), 2.59-2.41 (m, |
| | | | | | | 3H). |
| 95-64 | +++ | 2-15, 2-2 | B | (2R)-2-(3,4-difluoro-5- | 536.5 | (CD₃OD, 400 MHz) δ |
| (1^{st} | | | | methoxyphenyl)-1-[5-(2,3- | | (ppm): 7.32 (s, 2H), |
| eluting | | | | dihydro-1,4-benzodioxine-6- | | 7.02-7.00 (m, 1H), |
| isomer) | | | | sulfonyl)-1H,2H,3H,4H,5H,6H- | | 6.94-6.89 (m, 1H), |
| 95-65 | + | | | pyrrolo[3,4-c]pyrrol-2-yl]-3- | | 6.86-6.81 (m, 1H), |
| (2^{nd} | | | | (methylamino)propan-1-one; | | 4.42-4.38 (m, 1H), 4.31 |
| eluting | | | | (2S)-2-(3,4-difluoro-5- | | (br s, 4H), 4.21-4.17 |
| isomer) | | | | methoxyphenyl)-1-[5-(2,3- | | (m, 1H), 4.09 (br s, |
| | | | | dihydro-1,4-benzodioxine-6- | | 5H), 3.98-3.94 (m, |
| | | | | sulfonyl)-1H,2H,3H,4H,5H,6H- | | 1H), 3.90-3.87 (m, |
| | | | | pyrrolo[3,4-c]pyrrol-2-yl]-3- | | 4H), 3.23-3.15 (m, |
| | | | | (methylamino)propan-1-one | | 1H), 2.74-2.71 (m, |
| | | | | | | 1H), 2.39 (s, 3H). |
| 95-66 | ++++ | 2-13A, 2-2 | B | (2R)-2-(3-chloro-4,5- | 540.3 | (DMSO-d₆, 400 MHz) |
| (1^{st} | | | | difluorophenyl)-1-[5-(2,3-dihydro- | | δ (ppm): 7.42-7.37 (m, |
| eluting | | | | 1,4-benzodioxine-6-sulfonyl)- | | 2H), 7.31-7.26 (m, |
| isomer) | | | | 1H,2H,3H,4H,5H,6H-pyrrolo[3,4- | | 2H), 7.07 (d, J = 8.0 |
| 95-67 | + | | | c]pyrrol-2-yl]-3- | | Hz, 1H), 4.39-4.30 (m, |
| (2^{nd} | | | | (methylamino)propan-1-one; | | 5H), 4.11-3.92 (m, |
| eluting | | | | (2S)-2-(3-chloro-4,5- | | 8H), 3.08-2.97 (m, |
| isomer) | | | | difluorophenyl)-1-[5-(2,3-dihydro- | | 1H), 2.61-2.50 (m, |
| | | | | 1,4-benzodioxine-6-sulfonyl)- | | 1H), 2.23 (s, 3H), 1.17 |
| | | | | 1H,2H,3H,4H,5H,6H-pyrrolo[3,4- | | (br s, 1H). |
| | | | | c]pyrrol-2-yl]-3- | | |
| | | | | (methylamino)propan-1-one | | |
| 95-68 | +++ | 2-13, 2-2, | B | (2R)-1-[5-(2,3-dihydro-1,4- | 571.0 | (DMSO-d₆, 400 MHz) |
| (1^{st} | | 23-1 | | benzodioxine-6-sulfonyl)- | | δ (ppm): 7.50 (s, 1H), |
| eluting | | | | 1H,2H,3H,4H,5H,6H-pyrrolo[3,4- | | 7.28-7.23 (m, 2H), |
| isomer) | | | | c]pyrrol-2-yl]-2-(6-methoxy-2- | | 7.07-7.04 (m, 1H), 6.92 |
| 95-69 | ++ | | | methyl-1,3-benzothiazol-4-yl)-3- | | (s, 1H), 4.82-4.75 (m, |
| (2^{nd} | | | | (methylamino)propan-1-one; | | 1H), 4.41-4.38 (m, |
| eluting | | | | (2S)-1-[5-(2,3-dihydro-1,4- | | 1H), 4.32-4.30 (m, |
| isomer) | | | | benzodioxine-6-sulfonyl)- | | 4H), 4.18-3.89 (m, |
| | | | | 1H,2H,3H,4H,5H,6H-pyrrolo[3,4- | | 7H), 3.77 (s, 3H), 3.16- |
| | | | | c]pyrrol-2-yl]-2-(6-methoxy-2- | | 3.10 (m, 1H), 2.76 (s, |
| | | | | methyl-1,3-benzothiazol-4-yl)-3- | | 3H), 2.68-2.59 (m, |
| | | | | (methylamino)propan-1-one | | 1H), 2.76 (s, 3H), 1.91- |
| | | | | | | 1.81 (m, 1H). |
| 95-70 | ++++ | 27-1, 2-2 | B | (2R)-2-(3-cyclopropyl-4,5- | 546.4 | (DMSO-d₆, 400 MHz) |
| (1^{st} | | | | difluorophenyl)-1-[5-(2,3-dihydro- | | δ (ppm): 7.29-7.24 (m, |
| eluting | | | | 1,4-benzodioxine-6-sulfonyl)- | | 2H), 7.13-7.04 (m, |
| isomer) | | | | 1H,2H,3H,4H,5H,6H-pyrrolo[3,4- | | 2H), 6.74 (d, J = 6.4 |
| 95-71 | ++ | | | c]pyrrol-2-yl]-3- | | Hz, 1H), 4.36-4.30 (m, |
| (2^{nd} | | | | (methylamino)propan-1-one; | | 5H), 4.01-3.83 (m, |
| eluting | | | | (2S)-2-(3-cyclopropyl-4,5- | | 7H), 3.82-3.80 (m, |
| isomer) | | | | difluorophenyl)-1-[5-(2,3-dihydro- | | 1H), 3.00-2.95 (m, |
| | | | | 1,4-benzodioxine-6-sulfonyl)- | | 1H), 2.59-2.52 (m, |
| | | | | 1H,2H,3H,4H,5H,6H-pyrrolo[3,4- | | 1H), 2.22 (s, 3H), 2.05- |
| | | | | c]pyrrol-2-yl]-3- | | 2.00 (m, 1H), 1.02-1.00 |
| | | | | (methylamino)propan-1-one | | (m, 2H), 0.73-0.69 (m, |
| | | | | | | 2H). |
| 95-72 | +++ | 2-19, 2-2 | B | (2R)-2-(3-chloro-5-fluoro-4- | 552.4 | (DMSO-d₆, 400 MHz) |
| (1^{st} | | | | methoxyphenyl)-1-[5-(2,3- | | δ (ppm): 7.31-7.21 (m, |
| eluting | | | | dihydro-1,4-benzodioxine-6- | | 4H), 7.07 (d, J = 8.8 |
| isomer) | | | | sulfonyl)-1H,2H,3H,4H,5H,6H- | | Hz, 1H), 4.39-4.30 (m, |
| 95-73 | + | | | pyrrolo[3,4-c]pyrrol-2-yl]-3- | | 5H), 4.04-3.92 (m, |
| (2^{nd} | | | | (methylamino)propan-1-one; | | 8H), 3.86 (s, 3H), 3.08- |
| eluting | | | | (2S)-2-(3-chloro-5-fluoro-4- | | 3.03 (m, 1H), 2.71-2.66 |
| isomer) | | | | methoxyphenyl)-1-[5-(2,3- | | (m, 1H), 2.28 (s, 3H). |
| | | | | dihydro-1,4-benzodioxine-6- | | |
| | | | | sulfonyl)-1H,2H,3H,4H,5H,6H- | | |
| | | | | pyrrolo[3,4-c]pyrrol-2-yl]-3- | | |
| | | | | (methylamino)propan-1-one | | |
| 95-74 | ++++ | 13-4, 2-2 | B | (2R)-1-[5-(2,3-dihydro-1,4- | 518.4 | (DMSO-d₆, 400 MHz) |
| (1^{st} | | | | benzodioxine-6-sulfonyl)- | | δ (ppm): 7.29-7.24 (m, |
| eluting | | | | 1H,2H,3H,4H,5H,6H-pyrrolo[3,4- | | 2H), 7.14-7.06 (m, |
| isomer) | | | | c]pyrrol-2-yl]-2-(2-fluoro-5- | | 2H), 6.86-6.82 (m, |
| 95-75 | ++ | | | methoxyphenyl)-3- | | 2H), 4.42-4.38 (m, 1 |
| (2^{nd} | | | | (methylamino)propan-1-one; | | H), 4.33-4.31 (m, 4H), |
| eluting | | | | (2S)-1-[5-(2,3-dihydro-1,4- | | 4.13-4.10 (m, 1H), |
| isomer) | | | | benzodioxine-6-sulfonyl)- | | 4.02-3.95 (m, 6H), |
| | | | | 1H,2H,3H,4H,5H,6H-pyrrolo[3,4- | | 3.87-3.84 (m, 1H), 3.70 |
| | | | | c]pyrrol-2-yl]-2-(2-fluoro-5- | | (s, 3H), 3.13-3.08 (m, |
| | | | | methoxyphenyl)-3- | | 1H), 2.66-2.51 (m, |
| | | | | (methylamino)propan-1-one | | 1H), 2.27 (s, 3H). |
| 95-76 | ++++ | 13-2, 2-2 | B | (2S)-2-[3- | 536.7 | (CD₃OD, 400 MHz) δ |
| (1^{st} | | | | (difluoromethoxy)phenyl]-1-[5- | | (ppm): 7.41-7.39 (m, |
| eluting | | | | (2,3-dihydro-1,4-benzodioxine-6- | | 1H), 7.37-7.31 (m, |
| isomer) | | | | sulfonyl)-1H,2H,3H,4H,5H,6H- | | 2H), 7.20-7.18 (m, |
| 95-77 | ++ | | | pyrrolo[3,4-c]pyrrol-2-yl]-3- | | 1H), 7.12-7.07 (m, |
| (2^{nd} | | | | (methylamino)propan-1-one; | | 2H), 7.02-7.00 (d, J = 8 |
| eluting | | | | (2R)-2-[3- | | Hz, 1H), 6.65 (s, 1H), |
| isomer) | | | | (difluoromethoxy)phenyl]-1-[5- | | 4.41-4.38 (d, J = 12 Hz, |
| | | | | (2,3-dihydro-1,4-benzodioxine-6- | | 1H), 4.32-4.29 (m, |
| | | | | sulfonyl)-1H,2H,3H,4H,5H,6H- | | 4H), 4.21-4.18 (d, J = |
| | | | | pyrrolo[3,4-c]pyrrol-2-yl]-3- | | 12 Hz, 1H), 4.09-4.05 |
| | | | | (methylamino)propan-1-one | | (m, 5H), 3.95-3.88 (m, |
| | | | | | | 2H), 3.27-3.22 (m, |
| | | | | | | 1H), 2.76-2.71 (m, |
| | | | | | | 1H), 2.47 (s, 3H). |
| 95-78 | ++++ | 2-10, 2-2 | B | (2S)-2-[3- | 520.6 | (DMSO-d₆, 400 MHz) |
| (1^{st} | | | | (difluoromethyl)phenyl]-1-[5-(2,3- | | δ (ppm): 7.55-7.46 (m, |
| eluting | | | | dihydro-1,4-benzodioxine-6- | | 4H), 7.29-7.25 (m, |
| isomer) | | | | sulfonyl)-1H,2H,3H,4H,5H,6H- | | 2H), 7.07-7.05 (m, |
| 95-79 | ++ | | | pyrrolo[3,4-c]pyrrol-2-yl]-3- | | 1H), 7.01 (t, J = 56.0 |
| (2^{nd} | | | | (methylamino)propan-1-one; | | Hz, 1H), 4.44-4.41 (m, |
| eluting | | | | (2R)-2-[3- | | 1H), 4.31-4.19 (m, |
| isomer) | | | | (difluoromethyl)phenyl]-1-[5-(2,3- | | 4H), 4.09-3.78 (m, |
| | | | | dihydro-1,4-benzodioxine-6- | | 8H), 3.16-3.11 (m, |
| | | | | sulfonyl)-1H,2H,3H,4H,5H,6H- | | 1H), 2.68-2.64 (m, |
| | | | | pyrrolo[3,4-c]pyrrol-2-yl]-3- | | 1H), 2.11 (s, 3H). |
| | | | | (methylamino)propan-1-one | | |
| 95-80 | +++ | 2-11, 2-2 | B | (2S)-1-[5-(2,3-dihydro-1,4- | 538.5 | (DMSO-d₆, 400 MHz) |
| (1^{st} | | | | benzodioxine-6-sulfonyl)- | | δ (ppm): 7.67-7.62 (m, |
| eluting | | | | 1H,2H,3H,4H,5H,6H-pyrrolo[3,4- | | 1H), 7.57-7.54 (m, |
| isomer) | | | | c]pyrrol-2-yl]-3-(methylamino)-2- | | 2H), 7.53-7.51 (m, |
| 95-81 | + | | | [3- | | 1H), 7.29-7.25 (m, |
| (2^{nd} | | | | (trifluoromethyl)phenyl]propan-1- | | 2H), 7.08-7.05 (m, |
| eluting | | | | one; | | 1H), 4.44-4.40 (m, |
| isomer) | | | | (2R)-1-[5-(2,3-dihydro-1,4- | | 1H), 4.32-4.31 (m, |
| | | | | benzodioxine-6-sulfonyl)- | | 4H), 4.09-43.94 (m, |
| | | | | 1H,2H,3H,4H,5H,6H-pyrrolo[3,4- | | 8H), 3.14-3.09 (m, |
| | | | | c]pyrrol-2-yl]-3-(methylamino)-2- | | 1H), 2.71-2.66 (m, |
| | | | | [3- | | 1H), 2.28 (s, 3H). |
| | | | | (trifluoromethyl)phenyl]propan-1- | | |
| | | | | one | | |
| 95-82 | +++ | 2-22, 2-2 | B | (2R)-1-[5-(2,3-dihydro-1,4- | 530.4 | (DMSO-d₆, 400 MHz) |
| (1^{st} | | | | benzodioxine-6-sulfonyl)- | | δ (ppm): 7.42-7.40 (m, |
| eluting | | | | 1H,2H,3H,5H-pyrrolo[3,4- | | 2H), 7.14-7.13 (m, |
| isomer) | | | | c]pyrrol-2-yl]-2-(4-fluoro-2- | | 2H), 7.06-7.04 (m, |
| 95-83 | ++ | | | methoxy-5-methylphenyl)-3- | | 2H), 6.88-6.85 (m, |
| (2^{nd} | | | | (methylamino)propan-1-one; | | 1H), 4.67-4.64 (m, |
| eluting | | | | (2S)-1-[5-(2,3-dihydro-1,4- | | 1H), 4.39-4.26 (m, |
| isomer) | | | | benzodioxine-6-sulfonyl)- | | 7H), 4.15-4.12 (m, |
| | | | | 1H,2H,3H,5H-pyrrolo[3,4- | | 1H), 3.81 (s, 3H), 3.07- |
| | | | | c]pyrrol-2-yl]-2-(4-fluoro-2- | | 3.05 (m, 1H), 2.51-2.50 |
| | | | | methoxy-5-methylphenyl)-3- | | (m, 1H), 2.26 (s, 3H), |
| | | | | (methylamino)propan-1-one | | 2.10 (s, 3H). |
| 95-84 | + | 3-2, 15-1, | B | (2S)-2-(3-chlorophenyl)-1-[5-(2,3- | 504.4 | (DMSO-d₆, 300 MHz) |
| | | 16-1, 2-2 | | dihydro-1,4-benzodioxine-6- | | δ (ppm): 7.35-7.241 |
| | | | | sulfonyl)-1H,2H,3H,4H,5H,6H- | | (m, 6H), 7.07-7.04 (d, |
| | | | | pyrrolo[3,4-c]pyrrol-2-yl]-3- | | J = 8.1 Hz, 1H), 4.40- |
| | | | | (methylamino)propan-1-one | | 4.29 (m, 5H), 4.06-3.87 |
| | | | | | | (m, 8H), 3.06-3.00 (m, |
| | | | | | | 1H), 2.60-2.49 (m, |
| | | | | | | 1H), 2.23 (s, 3H), 1.58 |
| | | | | | | (s, 1H). |
| 95-85, | ++++ | 3-2, 15-1, | B, F | (2R)-2-(3-chlorophenyl)-1-[5-(2,3- | 504.4 | (DMSO-d₆, 300 MHz) |
| 99-1 | | 16-1, 2-2 | | dihydro-1,4-benzodioxine-6- | | δ (ppm): 7.35-7.241 |
| | | | | sulfonyl)-1H,2H,3H,4H,5H,6H- | | (m, 6H), 7.07-7.04 (d, |
| | | | | pyrrolo[3,4-c]pyrrol-2-yl]-3- | | J = 8.1 Hz, 1H), 4.40- |
| | | | | (methylamino)propan-1-one | | 4.29 (m, 5H), 4.06-3.87 |
| | | | | | | (m, 8H), 3.06-3.00 (m, |
| | | | | | | 1H), 2.60-2.49 (m, |
| | | | | | | 1H), 2.23 (s, 3H), 1.58 |
| | | | | | | (s, 1H). |
| 96-1 | +++ | 4-1, 2-2 | C | (2S)-2-(3-chlorophenyl)-1-[5-(2,3- | 522.4 | (DMSO-d₆, 400 MHz) |
| (1^{st} | | | | dihydro-1,4-benzodioxine-6- | | δ (ppm): 7.53-7.37 (m, |
| eluting | | | | sulfonyl)-1H,2H,3H,4H,5H,6H- | | 4H), 7.30-7.24 (m, |
| isomer) | | | | pyrrolo[3,4-c]pyrrol-2-yl]-2- | | 2H), 7.07 (d, J = 8.0 |
| 96-2 | + | | | fluoro-3-(methylamino)propan-1- | | Hz, 1H), 4.44-441 (m, |
| (2^{nd} | | | | one; | | 1H), 4.32-4.21 (m, |
| eluting | | | | (2R)-2-(3-chlorophenyl)-1-[5-(2,3- | | 4H), 4.10-4.08 (m, |
| isomer) | | | | dihydro-1,4-benzodioxine-6- | | 2H), 4.00-3.90 (m, |
| | | | | sulfonyl)-1H,2H,3H,4H,5H,6H- | | 5H), 3.39-3.29 (m, |
| | | | | pyrrolo[3,4-c]pyrrol-2-yl]-2- | | 1H). 3.08-2.92 (m, |
| | | | | fluoro-3-(methylamino)propan-1- | | 1H), 2.30 (s, 3H), 1.83- |
| | | | | one | | 1.79 (m, 1H). |
| 96-3 | ++ | 2-2, 68 | C | (2S)-2-(3-chlorophenyl)-1-[5-(2,3- | 520.0 | (DMSO-d₆, 400 MHz) |
| (1^{st} | | | | dihydro-1,4-benzodioxine-6- | | δ (ppm): 7.36-7.31 (m |
| eluting | | | | sulfonyl)-1H,2H,3H,4H,5H,6H- | | 6H), 7.07-7.04 (m, |
| isomer) | | | | pyrrolo[3,4-c]pyrrol-2-yl]-4- | | 1H), 4.41-4.29 (m, |
| | | | | (methylamino)butan-1-one; | | 5H), 4.01-3.83 (m, |
| 96-4 | ++++ | | | (2R)-2-(3-chlorophenyl)-1-[5-(2,3- | | 8H), 2.67-2.49 (m, |
| (2^{nd} | | | | dihydro-1,4-benzodioxine-6- | | 2H), 2.45-2.37 (m, |
| eluting | | | | sulfonyl)-1H,2H,3H,4H,5H,6H- | | 3H), 2.10-1.98 (m, |
| isomer) | | | | pyrrolo[3,4-c]pyrrol-2-yl]-4- | | 1H), 1.74-1.71 (m, |
| | | | | (methylamino)butan-1-one | | 1H). |
| 96-5 | ++ | 26-1, 2-2 | C | (2R)-2-cyclohexyl-1-[5-(2,3- | 476.5 | (CD₃OD, 400 MHz,) δ |
| (1^{st} | | | | dihydro-1,4-benzodioxine-6- | | (ppm): 7.40-7.32 (m, |
| eluting | | | | sulfonyl)-1H,2H,3H,4H,5H,6H- | | 2H), 7.08-7.01 (m, |
| isomer) | | | | pyrrolo[3,4-c]pyrrol-2-yl]-3- | | 1H), 4.32-4.21 (m, |
| 96-6 | −− | | | (methylamino)propan-1-one; | | 6H), 4.26-4.14 (m, |
| (2^{nd} | | | | (2S)-2-cyclohexyl-1-[5-(2,3- | | 6H), 3.17-3.11 (m, |
| eluting | | | | dihydro-1,4-benzodioxine-6- | | 1H), 2.97-2.92 (m, |
| isomer) | | | | sulfonyl)-1H,2H,3H,4H,5H,6H- | | 1H), 2.61-2.59 (m, |
| | | | | pyrrolo[3,4-c]pyrrol-2-yl]-3- | | 1H), 2.52 (s, 3H), 1.89- |
| | | | | (methylamino)propan-1-one | | 1.55 (m, 6H), 1.32-1.09 |
| | | | | | | (m, 5H). |
| 96-7 | ++ | 22-3, 2-2 | C | 1-[5-(2,3-dihydro-1,4- | 524.4 | (CDCl³, 400 MHz) δ |
| | | | | benzodioxine-6-sulfonyl)- | | (ppm): 8.23 (s, 1H), |
| | | | | 1H,2H,3H,4H,5H,6H-pyrrolo[3,4- | | 7.54-7.50 (m, 1H), 7.45 |
| | | | | c]pyrrol-2-yl]-3-(methylamino)-2- | | (s, 1H), 7.40-7.28 (m, |
| | | | | {6-methylimidazo[1,2-a]pyridin- | | 2H), 7.08-7.02 (m, |
| | | | | 8-yl}propan-1-one | | 1H), 6.96 (d, J = 8.4 |
| | | | | | | Hz, 1H), 4.43-4.39 (m, |
| | | | | | | 1H), 4.31-4.23 (m, |
| | | | | | | 6H), 4.14-4.05 (m, |
| | | | | | | 4H), 3.96-3.93 (m, |
| | | | | | | 1H), 3.78-3.75 (m, |
| | | | | | | 1H), 3.49-3.44 (m, |
| | | | | | | 1H), 2.93-2.89 (m, |
| | | | | | | 1H), 2.51-2.36 (m, |
| | | | | | | 3H), 2.35-2.33 (m, |
| | | | | | | 3H). |
| 96-8 | ++++ | 2-2, 75 | C | (2S)-2-(3,5-dichlorophenyl)-1-[5- | 552.3 | (DMSO-d₆, 400 MHz) |
| (1^{st} | | | | (2,3-dihydro-1,4-benzodioxine-6- | | δ (ppm): 7.49-7.48 (m, |
| eluting | | | | sulfonyl)-1H,2H,3H,4H,5H,6H- | | 1H), 7.37 (d, J = 1.6 |
| isomer) | | | | pyrrolo[3,4-c]pyrrol-2-yl]-4- | | Hz, 2H), 7.30-7.25 (m, |
| 96-9 | ++ | | | (methylamino)butan-1-one; | | 2H), 7.07 (d, J = 8.4 |
| (2^{nd} | | | | (2R)-2-(3,5-dichlorophenyl)-1-[5- | | Hz, 1H), 4.45-4.34 (m, |
| eluting | | | | (2,3-dihydro-1,4-benzodioxine-6- | | 1H), 4.32-4.29 (m, |
| isomer) | | | | sulfonyl)-1H,2H,3H,4H,5H,6H- | | 4H), 4.14-3.89 (m, |
| | | | | pyrrolo[3,4-c]pyrrol-2-yl]-4- | | 9H), 2.33-2.31 (m, |
| | | | | (methylamino)butan-1-one | | 2H), 2.20 (s, 3H), 2.10- |
| | | | | | | 1.98 (m, 1H), 1.74-1.65 |
| | | | | | | (m, 1H). |
| 96-10 | + | 20-1, 2-2 | C | (2S)-1-[5-(2,3-dihydro-1,4- | 476.4 | 1^{st}: (DMSO-d₆, 400 |
| (1^{st} | | | | benzodioxine-6-sulfonyl)- | | MHz) δ (ppm): 7.32- |
| eluting | | | | 1H,2H,3H,4H,5H,6H-pyrrolo[3,4- | | 7.28 (m, 2H), 7.10-7.08 |
| isomer) | | | | c]pyrrol-2-yl]-3-(methylamino)-2- | | (m, 1H), 4.43-4.33 (m, |
| | | | | [(1S,2R)-2- | | 5H), 4.19-4.16 (m, |
| | | | | methylcyclopentyl]propan-1-one; | | 1H), 4.08-3.94 (m, |
| | | | | (2S)-1-[5-(2,3-dihydro-1,4- | | 6H), 2.67-2.51 (m, |
| | | | | benzodioxine-6-sulfonyl)- | | 2H), 2.45-2.33 (m, |
| | | | | 1H,2H,3H,4H,5H,6H-pyrrolo[3,4- | | 1H), 2.20 (s, 3H), 1.99- |
| | | | | c]pyrrol-2-yl]-3-(methylamino)-2- | | 1.89 (m, 2H), 1.72-1.57 |
| | | | | [(1S,2S)-2- | | (m, 3H), 1.54-1.46 (m, |
| | | | | methylcyclopentyl]propan-1-one; | | 1H), 1.31-1.19 (m, |
| | | | | (2S)-1-[5-(2,3-dihydro-1,4- | | 2H), 0.66 (d, J = 6.8 |
| | | | | benzodioxine-6-sulfonyl)- | | Hz, 3H). |
| 96-11 | ++ | | | 1H,2H,3H,4H,5H,6H-pyrrolo[3,4- | | 2^{nd}: (DMSO-d₆, 400 |
| (2^{nd} | | | | c]pyrrol-2-yl]-3-(methylamino)-2- | | MHz) δ (ppm): 7.33- |
| eluting | | | | [(1R,2S)-2- | | 7.27 (m, 2H), 7.10-7.08 |
| isomer) | | | | methylcyclopentyl]propan-1-one; | | (m, 1H), 4.35-4.28 (m, |
| | | | | (2S)-1-[5-(2,3-dihydro-1,4- | | 5H), 4.19-4.16 (m, |
| | | | | benzodioxine-6-sulfonyl)- | | 1H), 4.04-3.92 (m, |
| | | | | 1H,2H,3H,4H,5H,6H-pyrrolo[3,4- | | 6H), 2.69-2.64 (m, |
| | | | | c]pyrrol-2-yl]-3-(methylamino)-2- | | 1H), 2.56-2.53 (m, |
| | | | | [(1R,2R)-2- | | 2H), 2.20 (s, 3H), 1.77- |
| | | | | methylcyclopentyl]propan-1-one; | | 1.50 (m, 5H), 1.47-1.40 |
| | | | | (2R)-1-[5-(2,3-dihydro-1,4- | | (m, 2H), 1.37-1.30 (m, |
| | | | | benzodioxine-6-sulfonyl)- | | 1H), 1.15-1.08 (m, |
| | | | | 1H,2H,3H,4H,5H,6H-pyrrolo[3,4- | | 1H), 0.95 (d, J = 6.4 |
| | | | | c]pyrrol-2-yl]-3-(methylamino)-2- | | Hz, 3H). |
| 96-12 | +++ | | | [(1S,2R)-2- | | 3^{rd}: (DMSO-d₆, 400 |
| (3^{rd} | | | | methylcyclopentyl]propan-1-one; | | MHz) δ (ppm): 7.34- |
| eluting | | | | (2R)-1-[5-(2,3-dihydro-1,4- | | 7.26 (m, 2H), 7.09 (d, |
| isomer) | | | | benzodioxine-6-sulfonyl)- | | J = 8.4 Hz, 1H), 4.39- |
| | | | | 1H,2H,3H,4H,5H,6H-pyrrolo[3,4- | | 4.20 (m, 6H), 4.04-3.92 |
| | | | | c]pyrrol-2-yl]-3-(methylamino)-2- | | (m, 6H), 2.68-2.52 (m, |
| | | | | [(1S,2S)-2- | | 2H), 2.49-2.42 (m, |
| | | | | methylcyclopentyl]propan-1-one; | | 1H), 2.20 (s, 3H), 2.10- |
| | | | | (2R)-1-[5-(2,3-dihydro-1,4- | | 2.03 (m, 1H), 1.72-1.63 |
| | | | | benzodioxine-6-sulfonyl)- | | (m, 1H), 1.72-1.61 (m, |
| | | | | 1H,2H,3H,4H,5H,6H-pyrrolo[3,4- | | 1H), 1.61-1.49 (m, |
| | | | | c]pyrrol-2-yl]-3-(methylamino)-2- | | 1H), 1.44-1.36 (m, |
| | | | | [(1R,2R)-2- | | 2H), 1.34-1.26 (m, |
| | | | | methylcyclopentyl]propan-1-one; | | 1H), 1.24-1.16 (m, |
| | | | | (2R)-1-[5-(2,3-dihydro-1,4- | | 1H), 0.76 (d, J = 7.2 |
| | | | | benzodioxine-6-sulfonyl)- | | Hz, 3H). |
| 96-13 | ++ | | | 1H,2H,3H,4H,5H,6H-pyrrolo[3,4- | | 4^{th}: (DMSO-d₆, 400 |
| (4^{th} | | | | c]pyrrol-2-yl]-3-(methylamino)-2- | | MHz) δ (ppm): 7.32- |
| eluting | | | | [(1R,2S)-2- | | 7.28 (m, 2H), 7.09 (d, |
| isomer) | | | | methylcyclopentyl]propan-1-one | | J = 8.4 Hz, 1H), 4.34- |
| | | | | | | 4.29 (m, 5H), 4.22-4.18 |
| | | | | | | (m, 1H), 4.05 (s, 5H), |
| | | | | | | 3.96-3.92 (m, 1H), |
| | | | | | | 2.71-2.66 (m, 1H), 2.47 |
| | | | | | | (s, 2H), 2.20 (s, 3H), |
| | | | | | | 1.75-1.67 (m, 2H), |
| | | | | | | 1.62-1.43 (m, 4H), |
| | | | | | | 1.32-1.22 (m, 1H), |
| | | | | | | 1.13-1.05 (m, 1H), 0.85 |
| | | | | | | (d, J = 6.4 Hz, 3H). |
| 96-14 | + | | | | | 5^{th}: (DMSO-d₆, 400 |
| (5^{th} | | | | | | MHz) δ (ppm): 7.33- |
| eluting | | | | | | 7.29 (m, 2H), 7.10 (d, |
| isomer) | | | | | | J = 8.4 Hz, 1H), 4.44- |
| | | | | | | 4.33 (m, 5H), 4.20-4.93 |
| | | | | | | (m, 7H), 2.68-2.55 (m, |
| | | | | | | 2H), 2.45-2.39 (m, |
| | | | | | | 1H), 2.20 (s, 3H), 2.05- |
| | | | | | | 1.88 (m, 3H), 1.73-1.44 |
| | | | | | | (m, 4H), 1.32-1.19 (m, |
| | | | | | | 2H), 0.67 (d, J = 6.8 |
| | | | | | | Hz, 3H). |
| 96-15 | + | | | | | 6^{th}: (DMSO-d₆, 400 |
| (6^{th} | | | | | | MHz) δ (ppm): 7.32- |
| eluting | | | | | | 7.28 (m, 2H), 7.09 (d, |
| isomer) | | | | | | J = 8.4 Hz, 1H), 4.34- |
| | | | | | | 4.29 (m, 5H), 4.19-4.16 |
| | | | | | | (m, 1H), 4.04 (s, 5H), |
| | | | | | | 3.96-3.92 (m, 1H), |
| | | | | | | 2.72-2.67 (m, 1H), |
| | | | | | | 2.58-2.55 (m, 2H), 2.22 |
| | | | | | | (s, 3H), 1.77-1.50 (m, |
| | | | | | | 4H), 1.47-1.40 (m, |
| | | | | | | 2H), 1.38-1.29 (m, |
| | | | | | | 1H), 1.16-1.08 (m, |
| | | | | | | 1H), 0.95 (d, J = 6.4 |
| | | | | | | Hz, 3H). |
| 96-16 | + | | | | | 7^{th}: (DMSO-d₆, 400 |
| (7^{th} | | | | | | MHz) δ (ppm): 7.32- |
| eluting | | | | | | 7.28 (m, 2H), 7.09 (d, |
| isomer) | | | | | | J = 8.4 Hz, 1H), 4.34- |
| | | | | | | 4.28 (m, 5H), 4.22-4.18 |
| | | | | | | (m, 1H), 4.05-4.03 (m, |
| | | | | | | 5H), 3.96-3.91 (m, |
| | | | | | | 1H), 2.70-2.64 (m, |
| | | | | | | 1H), 2.48-2.45 (m, |
| | | | | | | 2H), 2.19 (s, 3H), 1.76- |
| | | | | | | 1.66 (m, 2H), 1.61-1.43 |
| | | | | | | (m, 4H), 1.32-1.22 (m, |
| | | | | | | 1H), 1.12-1.04 (m, |
| | | | | | | 1H), 0.84 (d, J = 6.4 |
| | | | | | | Hz, 3H). |
| 96-17 | + | | | | | 8^{th}: (DMSO-d₆, 400 |
| (8^{th} | | | | | | MHz) δ (ppm): 7.32- |
| eluting | | | | | | 7.28 (m, 2H), 7.09 (d, |
| isomer) | | | | | | J = 8.4 Hz, 1H), 4.34- |
| | | | | | | 4.22 (m, 6H), 4.05-4.03 |
| | | | | | | (m, 5H), 3.97-3.92 (m, |
| | | | | | | 1H), 2.67-2.57 (m, |
| | | | | | | 2H), 2.48-2.46 (m, |
| | | | | | | 1H), 2.23 (s, 3H), 2.10- |
| | | | | | | 2.02 (m, 1H), 1.82-1.73 |
| | | | | | | (m, 1H), 1.71-1.62 (m, |
| | | | | | | 1H), 1.60-1.51 (m, |
| | | | | | | 1H), 1.46-1.35 (m, |
| | | | | | | 2H), 1.33-1.11 (m, |
| | | | | | | 2H), 0.77 (d, J = 6.8 |
| | | | | | | Hz, 3H). |
| 96-18 | +++ | 2-2 | C | (2R)-1-[5-(2,3-dihydro-1,4- | 470.3 | (DMSO-d₆, 300 MHz) |
| | | | | benzodioxine-6-sulfonyl)- | | δ (ppm): 7.30-7.23 (m, |
| | | | | 1H,2H,3H,4H,5H,6H-pyrrolo[3,4- | | 7H), 7.05 (d, J = 9.0 |
| | | | | c]pyrrol-2-yl]-3-(methylamino)-2- | | Hz, 1H), 4.45-4.23 (m, |
| | | | | phenylpropan-1-one; or (2S)-1-[5- | | 5H), 4.04-3.84 (m, |
| | | | | (2,3-dihydro-1,4-benzodioxine-6- | | 8H), 3.11-3.05 (m, |
| | | | | sulfonyl)-1H,2H,3H,4H,5H,6H- | | 1H), 2.59-2.57 (m, |
| | | | | pyrrolo[3,4-c]pyrrol-2-yl]-3- | | 1H), 2.24 (s, 3H). |
| | | | | (methylamino)-2-phenylpropan-1- | | |
| | | | | one | | |
| 97-1 | +++ | 2-2, 21-1, | D | (2S)-1-[5-(2,3-dihydro-1,4- | 498.3 | (DMSO-d₆, 400 MHz) |
| (1^{st} | | 22, 63-6 | | benzodioxine-6-sulfonyl)- | | δ (ppm): 7.29-7.26 (m, |
| eluting | | | | 1H,2H,3H,4H,5H,6H-pyrrolo[3,4- | | 1H), 7.25-7.24 (m, |
| isomer) | | | | c]pyrrol-2-yl]-2-hydroxy-2-(2- | | 1H), 7.19-7.15 (m, 2H), |
| | | | | methyl-2,3-dihydro-1H-isoindol- | | 7.13-7.10 (m, 1H), |
| | | | | 4-yl)ethan-1-one | | 7.08-7.06 (m, 1H), 5.52 |
| 97-2 | ++ | | | (2R)-1-[5-(2,3-dihydro-1,4- | | (d, J = 6.0 Hz, 1H), |
| (2^{nd} | | | | benzodioxine-6-sulfonyl)- | | 5.15 (d, J = 5.6 Hz, |
| eluting | | | | 1H,2H,3H,4H,5H,6H-pyrrolo[3,4- | | 1H), 4.34-4.30 (m, |
| isomer) | | | | c]pyrrol-2-yl]-2-hydroxy-2-(2- | | 5H), 4.13-4.19 (m, |
| | | | | methyl-2,3-dihydro-1H-isoindol- | | 6H), 3.99-3.72 (m, |
| | | | | 4-yl)ethan-1-one | | 5H), 2.68-2.33 (m, |
| | | | | | | 3H). |
| 97-3 | ++++ | 30-1, 2-2 | D | (2R)-1-[5-(2,3-dihydro-1,4- | 541.4 | (DMSO-d₆, 300 MHz) |
| (1^{st} | | | | benzodioxine-6-sulfonyl)- | | δ (ppm): 7.91-7.88 (m, |
| eluting | | | | 1H,2H,3H,4H,5H,6H-pyrrolo[3,4- | | 1H), 7.38-7.22 (m, |
| isomer) | | | | c]pyrrol-2-yl]-2-(2-methyl-1,3- | | 4H), 7.40 (d, J = 8.4 |
| 97-4 | ++ | | | benzothiazol-4-yl)-3- | | Hz, 1H), 4.90-4.85 (m, |
| (2^{nd} | | | | (methylamino)propan-1-one; | | 1H), 4.42-4.28 (m, |
| eluting | | | | (2S)-1-[5-(2,3-dihydro-1,4- | | 5H), 4.10-3.92 (m, |
| isomer) | | | | benzodioxine-6-sulfonyl)- | | 7H), 3.20-3.14 (m, |
| | | | | 1H,2H,3H,4H,5H,6H-pyrrolo[3,4- | | 1H), 2.82 (s, 3H), 2.71- |
| | | | | c]pyrrol-2-yl]-2-(2-methyl-1,3- | | 2.66 (m, 1H), 2.27 (s, |
| | | | | benzothiazol-4-yl)-3- | | 3H). |
| | | | | (methylamino)propan-1-one | | |
| 97-5 | +++ | 36-1, 2-2, | D | (2S)-1-[5-(2,3-dihydro-1,4- | 512.2 | (CD₃OD, 300 MHz) δ |
| (1^{st} | | 63-8 | | benzodioxine-6-sulfonyl)- | | (ppm): 7.45-7.34 (m, |
| eluting | | | | 1H,2H,3H,4H,5H,6H-pyrrolo[3,4- | | 5H), 7.34-7.24 (m, |
| isomer) | | | | c]pyrrol-2-yl]-2-[(1- | | 2H), 7.03-6.95 (m, |
| 97-6 | ++ | | | methylazetidin-3-yl)oxy]-2- | | 1H), 5.08 (s, 1H), 4.32- |
| (2^{nd} | | | | phenylethan-1-one; | | 4.11 (m, 7H), 4.15-3.99 |
| eluting | | | | (2R)-1-[5-(2,3-dihydro-1,4- | | (m, 5H), 3.93-3.79 (m, |
| isomer) | | | | benzodioxine-6-sulfonyl)- | | 1H), 3.75-3.63 (m, |
| | | | | 1H,2H,3H,4H,5H,6H-pyrrolo[3,4- | | 1H), 3.58-3.41 (m, |
| | | | | c]pyrrol-2-yl]-2-[(1- | | 1H), 3.29-3.18 (m, |
| | | | | methylazetidin-3-yl)oxy]-2- | | 1H), 3.08-2.95 (m, |
| | | | | phenylethan-1-one | | 1H), 2.39 (s, 3H). |
| 97-7 | +++ | 6-1, 2-2, | D | (2S)-1-[5-(2,3-dihydro-1,4- | 516.5 | (CDCl₃, 400 MHz) δ |
| (1^{st} | | 63-6 | | benzodioxine-6-sulfonyl)- | | (ppm): 7.54-7.31(m, |
| eluting | | | | 1H,2H,3H,4H,5H,6H-pyrrolo[3,4- | | 2H), 7.16 (br s, 1H), |
| isomer) | | | | c]pyrrol-2-yl]-2-(7-fluoro-2- | | 7.03-6.94 (m, 2H), 4.99 |
| 97-8 | + | | | methyl-2,3-dihydro-1H-isoindol- | | (s, 1H), 4.64-4.30 (m, |
| (2^{nd} | | | | 4-yl)-2-hydroxyethan-1-one; | | 6H), 4.22-4.05 (m, |
| eluting | | | | (2R)-1-[5-(2,3-dihydro-1,4- | | 8H), 3.99-3.88 (m, |
| isomer) | | | | benzodioxine-6-sulfonyl)- | | 2H), 3.54-3.51(m, 1H), |
| | | | | 1H,2H,3H,4H,5H,6H-pyrrolo[3,4- | | 2.75-2.62 (m, 3H). |
| | | | | c]pyrrol-2-yl]-2-(7-fluoro-2- | | |
| | | | | methyl-2,3-dihydro-1H-isoindol- | | |
| | | | | 4-yl)-2-hydroxyethan-1-one | | |
| 97-9 | ++++ | 7-2, 2-2, | D | (2S)-1-[5-(2,3-dihydro-1,4- | 512.5 | (DMSO-d₆, 400 MHz) |
| (1^{st} | | 63-4 | | benzodioxine-6-sulfonyl)- | | δ (ppm): 7.29-7.23 (m, |
| eluting | | | | 1H,2H,3H,4H,5H,6H-pyrrolo[3,4- | | 2H), 7.11-7.04 (m, |
| isomer) | | | | c]pyrrol-2-yl]-2-hydroxy-2-(2- | | 3H), 6.96-6.94 (m, |
| 97-10 | +++ | | | methyl-1,2,3,4- | | 1H), 5.33 (d, J = 6.8 |
| (2^{nd} | | | | tetrahydroisoquinolin-8-yl)ethan- | | Hz, 1H), 5.19 (d, J = |
| eluting | | | | 1-one; | | 6.4 Hz, 1H), 4.34-4.25 |
| isomer) | | | | (2R)-1-[5-(2,3-dihydro-1,4- | | (m, 5H), 4.15-3.97 (m, |
| | | | | benzodioxine-6-sulfonyl)- | | 6H), 3.62-3.53 (m, |
| | | | | 1H,2H,3H,4H,5H,6H-pyrrolo[3,4- | | 3H), 2.85-2.82 (m, |
| | | | | c]pyrrol-2-yl]-2-hydroxy-2-(2- | | 2H), 2.68-2.51 (m, |
| | | | | methyl-1,2,3,4- | | 2H), 2.36 (s, 3H). |
| | | | | tetrahydroisoquinolin-8-yl)ethan- | | |
| | | | | 1-one | | |
| 97-11 | +++ | 2-2, 21-1, | D | (2S)-1-[5-(2,3-dihydro-1,4- | 540.4 | (DMSO-d₆, 400 MHz) |
| (1^{st} | | 22, 63-6 | | benzodioxine-6-sulfonyl)- | | δ (ppm): 7.26-7.24 (m, |
| eluting | | | | 1H,2H,3H,4H,5H,6H-pyrrolo[3,4- | | 2H), 7.20-7.19 (m, |
| isomer) | | | | c]pyrrol-2-yl]-2-hydroxy-2-[2- | | 3H), 7.06 (d, J = 8.4 Hz, |
| 97-12 | + | | | (oxetan-3-yl)-2,3-dihydro-1H- | | 1H), 5.52 (d, J = 4.0 |
| (2^{nd} | | | | isoindol-4-yl]ethan-1-one; | | Hz, 1H), 5.16 (d, J = |
| eluting | | | | (2R)-1-[5-(2,3-dihydro-1,4- | | 6.0 Hz, 1H), 4.64-4.62 |
| isomer) | | | | benzodioxine-6-sulfonyl)- | | (m, 2H), 4.56-4.55 (m, |
| | | | | 1H,2H,3H,4H,5H,6H-pyrrolo[3,4- | | 2H), 4.33-4.31 (m, |
| | | | | c]pyrrol-2-yl]-2-hydroxy-2-[2- | | 5H), 4.02-3.99 (m, |
| | | | | (oxetan-3-yl)-2,3-dihydro-1H- | | 8H), 3.85 (s, 3H), 3.84- |
| | | | | isoindol-4-yl]ethan-1-one | | 3.70 (m, 1H). |
| 97-13 | ++++ | 33-2, 2-2, | D | (2S)-2-(3-chlorophenyl)-1-[5-(2,3- | 550.0 | (DMSO-d₆, 400 MHz) |
| (1^{st} | | 63-10 | | dihydro-1,4-benzodioxine-6- | | δ (ppm): 7.44-7.38 (m, |
| eluting | | | | sulfonyl)-1H,2H,3H,4H,5H,6H- | | 3H), 7.37-7.25 (m, |
| isomer) | | | | pyrrolo[3,4-c]pyrrol-2-yl]-2-[2- | | 3H), 7.10-7.06 (m, |
| 97-14 | ++ | | | (dimethylamino)ethoxy]ethan-1- | | 1H), 5.13 (s, 1H), 4.36- |
| (2^{nd} | | | | one; | | 4.30 (m, 5H), 4.20-3.94 |
| eluting | | | | (2R)-2-(3-chlorophenyl)-1-[5-(2,3- | | (m, 7H), 3.57-3.51 (m, |
| isomer) | | | | dihydro-1,4-benzodioxine-6- | | 1H), 3.47-3.43 (m, |
| | | | | sulfonyl)-1H,2H,3H,4H,5H,6H- | | 1H), 2.44-2.41 (m, |
| | | | | pyrrolo[3,4-c]pyrrol-2-yl]-2-[2- | | 2H), 2.12 (s, 6H). |
| | | | | (dimethylamino)ethoxy]ethan-1- | | |
| | | | | one | | |
| 97-15 | ++++ | 63-9, 66-4, | D | (2S)-1-[5-(2,3-dihydro-1,4- | 557.0 | (CDCl₃, 400 MHz) δ |
| (1^{st} | | 83-2, 87 | | benzodioxine-6-sulfonyl)- | | (ppm): 7.36-7.31 (m, |
| eluting | | | | 1H,2H,3H,4H,5H,6H-pyrrolo[3,4- | | 2H), 7.28-7.23 (m, |
| isomer) | | | | c]pyrrol-2-yl]-2-hydroxy-2-[3-(4- | | 1H), 7.01-6.97 (m, |
| 97-16 | ++ | | | methylpiperazin-1- | | 2H), 6.90-6.88 (m, |
| (2^{nd} | | | | yl)phenyl]ethane-1-thione; | | 2H), 5.18-5.15 (m, |
| eluting | | | | (2R)-1-[5-(2,3-dihydro-1,4- | | 1H), 5.05-5.03 (m, |
| isomer) | | | | benzodioxine-6-sulfonyl)- | | 1H), 4.66-4.61 (m, |
| | | | | 1H,2H,3H,4H,5H,6H-pyrrolo[3,4- | | 1H), 4.49-4.44 (m, |
| | | | | c]pyrrol-2-yl]-2-hydroxy-2-[3-(4- | | 1H), 4.37-4.30 (m, |
| | | | | methylpiperazin-1- | | 5H), 4.20-4.11 (m, |
| | | | | yl)phenyl]ethane-1-thione | | 3H), 4.04-4.01 (m, |
| | | | | | | 1H), 3.92-3.87 (m, |
| | | | | | | 1H), 3.31 (s, 4H), 2.81- |
| | | | | | | 2.71 (m, 4H), 2.55-2.46 |
| | | | | | | (m, 3H). |
| 97-17 | −− | 66-2, 2-2, | D | (2S)-1-[5-(2,3-dihydro-1,4- | 565.0 | (DMSO-d6, 400 MHz) |
| (1^{st} | | 63-9 | | benzodioxine-6-sulfonyl)- | | δ (ppm): 7.30-7.21 (m, |
| eluting | | | | 1H,2H,3H,4H,5H,6H-pyrrolo[3,4- | | 2H), 7.13 (d, J = 7.6 |
| isomer) | | | | c]pyrrol-2-yl]-2-hydroxy-2-(3-{5- | | Hz, 1H), 7.06 (d, J = |
| 97-18 | ++++ | | | methyl-1H,2H,3H,4H,5H,6H- | | 8.4 Hz, 1H), 6.58 (d, |
| (2^{nd} | | | | pyrrolo[3,4-c]pyrrol-2- | | J = 7.6 Hz, 1H), 6.50 (d, |
| eluting | | | | yl}phenyl)ethan-1-one; | | J = 2.0 Hz, 1H), 6.42 |
| isomer) | | | | (2R)-1-[5-(2,3-dihydro-1,4- | | (d, J = 8.0 Hz, 1H), |
| | | | | benzodioxine-6-sulfonyl)- | | 5.47 (d, J = 6.4 Hz, |
| | | | | 1H,2H,3H,4H,5H,6H-pyrrolo[3,4- | | 1H), 5.06 (d, J = 5.6 |
| | | | | c]pyrrol-2-yl]-2-hydroxy-2-(3-{5- | | Hz, 1H), 4.33-4.29 (m, |
| | | | | methyl-1H,2H,3H,4H,5H,6H- | | 4H), 4.25-4.20 (m, |
| | | | | pyrrolo[3,4-c]pyrrol-2- | | 1H), 4.11-4.07 (m, |
| | | | | yl}phenyl)ethan-1-one | | 1H), 3.98-3.87 (m, |
| | | | | | | 10H), 3.48 (s, 4H), |
| | | | | | | 2.48 (s, 3H). |
| 97-19 | ++++ | 2-2, 63-5 | D | (2R)-1-[5-(2,3-dihydro-1,4- | 484.4 | (CD₃OD, 300 MHz) δ |
| | | | | benzodioxine-6-sulfonyl)- | | (ppm): 7.43-7.30 (m, |
| | | | | 1H,2H,3H,4H,5H,6H-pyrrolo[3,4- | | 7H), 7.02-6.99 (m, |
| | | | | c]pyrrol-2-yl]-3-(dimethylamino)- | | 1H), 4.41-4.09 (m, |
| | | | | 2-phenylpropan-1-one; or | | 12H), 3.78-3.56 (m, |
| | | | | (2S)-1-[5-(2,3-dihydro-1,4- | | 2H), 3.00-2.95 (m, |
| | | | | benzodioxine-6-sulfonyl)- | | 1H), 2.68 (s, 6H). |
| | | | | 1H,2H,3H,4H,5H,6H-pyrrolo[3,4- | | |
| | | | | c]pyrrol-2-yl]-3-(dimethylamino)- | | |
| | | | | 2-phenylpropan-1-one | | |
| 97-20 | ++++ | 2-2, 66, | D | (2S)-1-[5-(2,3-dihydro-1,4- | 567.5 | (CDCl₃, 300 MHz) δ |
| | | 63-9 | | benzodioxine-6-sulfonyl)- | | (ppm): 7.36-7.19 (m, |
| | | | | 1H,2H,3H,4H,5H,6H-pyrrolo[3,4- | | 3H), 6.98 (d, J = 8.4 |
| | | | | c]pyrrol-2-yl]-2-hydroxy-2-(3-{5- | | Hz, 1H), 6.71-6.60 (m, |
| | | | | methyl-octahydropyrrolo[3,4- | | 3H), 4.93 (s, 1H), 4.34- |
| | | | | c]pyrrol-2-yl}phenyl)ethan-1-one; | | 3.99 (m, 11H), 3.68- |
| | | | | or (2R)-1-[5-(2,3-dihydro-1,4- | | 3.64 (m, 1H), 3.40-3.27 |
| | | | | benzodioxine-6-sulfonyl)- | | (m, 4H), 3.07-2.94 (m, |
| | | | | 1H,2H,3H,4H,5H,6H-pyrrolo[3,4- | | 4H), 2.59-2.57 (m, |
| | | | | c]pyrrol-2-yl]-2-hydroxy-2-(3-{5- | | 2H), 2.46 (s, 3H). |
| | | | | methyl-octahydropyrrolo[3,4- | | |
| | | | | c]pyrrol-2-yl}phenyl)ethan-1-one | | |
| 98-1 | ++++ | 7-1, 2-2 | E | (2S)-2-(2-cyclopropyl-2,3- | 524.5 | (CDCl₃, 400 MHz) δ |
| (1^{st} | | | | dihydro-1H-isoindol-4-yl)-1-[5- | | (ppm): 7.35-7.28 (m, |
| eluting | | | | (2,3-dihydro-1,4-benzodioxine-6- | | 2H), 7.25-7.22 (m, |
| isomer) | | | | sulfonyl)-1H,2H,3H,4H,5H,6H- | | 2H), 7.14-7.12 (m, |
| 98-2 | + | | | pyrrolo[3,4-c]pyrrol-2-yl]-2- | | 1H), 7.03-6.97 (m, |
| (2^{nd} | | | | hydroxyethan-1-one; | | 1H), 5.02 (s, 1H), 4.37- |
| eluting | | | | (2R)-2-(2-cyclopropyl-2,3- | | 4.32 (m, 6H), 4.32-4.04 |
| isomer) | | | | dihydro-1H-isoindol-4-yl)-1-[5- | | (m, 8H), 3.97-3.85 (m, |
| | | | | (2,3-dihydro-1,4-benzodioxine-6- | | 1H), 3.52-3.48 (m, |
| | | | | sulfonyl)-1H,2H,3H,4H,5H,6H- | | 1H), 2.16-2.03 (s, 1H), |
| | | | | pyrrolo[3,4-c]pyrrol-2-yl]-2- | | 0.61-0.57 (br s, 4H). |
| | | | | hydroxyethan-1-one | | |
| 99-2 | ++ | 36-1, 2-2 | F | (2S)-2-(azetidin-3-yloxy)-1-[5- | 498.4 | (DMSO-d₆, 400 MHz) |
| (1^{st} | | | | (2,3-dihydro-1,4-benzodioxine-6- | | δ (ppm): 7.36-7.31 (m, |
| eluting | | | | sulfonyl)-1H,2H,3H,4H,5H,6H- | | 5H), 7.28-7.23 (m, |
| isomer) | | | | pyrrolo[3.4-c]pyrrol-2-yl]-2- | | 2H), 7.04 (d, J = 8.4 |
| 99-3 | +++ | | | phenylethan-1-one; | | Hz, 1H), 5.00 (s, 1H), |
| (2^{nd} | | | | (2R)-2-(azetidin-3-yloxy)-1-[5- | | 4.32-3.92 (m, 15H), |
| eluting | | | | (2,3-dihydro-1,4-benzodioxine-6- | | 3.43-3.44 (m, 2H). |
| isomer) | | | | sulfonyl)-1H,2H,3H,4H,5H,6H- | | |
| | | | | pyrrolo[3,4-c]pyrrol-2-yl]-2- | | |
| | | | | phenylethan-1-one | | |
| 99-4 | +++ | 3-2, 2-1 | F | (2R)-2-(3-chlorophenyl)-1-[5-(2,3- | 502.4 | (DMSO-d₆, 400 MHz) |
| (1^{st} | | | | dihydro-1,4-benzodioxine-6- | | δ (ppm): 7.49-7.40 (m, |
| eluting | | | | sulfonyl)-1H,2H,3H,5H- | | 3H), 7.39-7.30 (m, |
| isomer) | | | | pyrrolo[3,4-c]pyrrol-2-yl]-3- | | 3H), 7.15-7.13 (m, |
| 99-5 | + | | | (methylamino)propan-1-one; | | 2H), 7.07-7.05 (m, |
| (2^{nd} | | | | (2S)-2-(3-chlorophenyl)-1-[5-(2,3- | | 1H), 4.76-4.72 (m, |
| eluting | | | | dihydro-1,4-benzodioxine-6- | | 1H), 4.40-4.24 (m, |
| isomer) | | | | sulfonyl)-1H,2H,3H,5H- | | 7H), 4.07-4.01 (m, |
| | | | | pyrrolo[3,4-c]pyrrol-2-yl]-3- | | 1H), 3.10-3.05 (m, |
| | | | | (methylamino)propan-1-one | | 1H), 2.67-2.64 (m, |
| | | | | | | 1H), 2.26 (s, 3H). |
| 99-6 | + | 33-1, 2-2 | F | (2R)-2-(3-chlorophenyl)-1-[5-(2,3- | 530.4 | (DMSO-d₆, 400 MHz) |
| (1^{st} | | | | dihydro-1,4-benzodioxine-6- | | δ (ppm): 7.42 (s, 1H) |
| eluting | | | | sulfonyl)-1H,2H,3H,4H,5H,6H- | | 7.33-7.24 (m, 5H), |
| isomer) | | | | pyrrolo[3,4-c]pyrrol-2-yl]-2-[(2S)- | | 7.07-7.05 (m, 1H), |
| 99-7 | +++ | | | pyrrolidin-2-yl]ethan-1-one; | | 4.43-4.32 (m, 1H), |
| (2^{nd} | | | | (2S)-2-(3-chlorophenyl)-1-[5-(2,3- | | 4.31-4.29 (m, 4H), |
| eluting | | | | dihydro-1,4-benzodioxine-6- | | 4.06-3.89 (m, 7H), |
| isomer) | | | | sulfonyl)-1H,2H,3H,4H,5H,6H- | | 3.59-3.50 (m, 2H), |
| | | | | pyrrolo[3,4-c]pyrrol-2-yl]-2-[(2R)- | | 2.79-2.75 (m, 2H), 2.50 |
| | | | | pyrrolidin-2-yl]ethan-1-one | | (s, 1H), 1.65-1.60 (m, |
| | | | | | | 1H), 1.54-1.47 (m, |
| | | | | | | 1H), 1.34-1.29 (m, |
| | | | | | | 1H), 1.19-1.10 (m, |
| | | | | | | 1H). |
| 99-8 | ++++ | 33-1, 2-2 | F | (2S)-2-(3-chlorophenyl)-1-[5-(2,3- | 530.3 | (DMSO-d₆, 400 MHz) |
| (1^{st} | | | | dihydro-1,4-benzodioxine-6- | | δ (ppm): 7.38 (s, 1H), |
| eluting | | | | sulfonyl)-1H,2H,3H,4H,5H,6H- | | 7.33-7.23 (m, 5H), |
| isomer) | | | | pyrrolo[3,4-c]pyrrol-2-yl]-2-[(2S)- | | 7.07-7.05 (m, 1H), |
| 99-9 | ++ | | | pyrrolidin-2-yl]ethan-1-one; | | 4.42-4.31 (m, 1H), |
| (2^{nd} | | | | (2R)-2-(3-chlorophenyl)-1-[5-(2,3- | | 4.30-4.29 (m, 4H), |
| eluting | | | | dihydro-1,4-benzodioxine-6- | | 4.03-3.95 (m, 7H), |
| isomer) | | | | sulfonyl)-1H,2H,3H,4H,5H,6H- | | 3.53-3.43 (m, 2H), |
| | | | | pyrrolo[3,4-c]pyrrol-2-yl]-2-[(2R)- | | 2.77-2.72 (m, 1H), |
| | | | | pyrrolidin-2-yl]ethan-1-one | | 2.66-2.59 (m, 1H), 1.85 |
| | | | | | | (s, 1H), 1.84-1.81 (m, |
| | | | | | | 1H), 1.66-1.57 (m, |
| | | | | | | 2H), 1.28-1.19 (m, |
| | | | | | | 1H). |
| 99-10 | ++ | 2-2, 41-1 | F | (2S)-2-(3-chloro-4,5- | 570.3 | (DMSO-d₆, 400 MHz) |
| (1^{st} | | | | difluorophenyl)-1-[5-(2,3-dihydro- | | δ (ppm): 7.47-7.42 (m, |
| eluting | | | | 1,4-benzodioxine-6-sulfonyl)- | | 2H), 7.33-7.26 (m, |
| isomer) | | | | 1H,2H,3H,4H,5H,6H-pyrrolo[3,4- | | 2H), 7.10-7.07 (m, |
| 99-11 | +++ | | | c]pyrrol-2-yl]-2-[2- | | 1H), 5.14 (s, 1H), 4.33- |
| (2^{nd} | | | | (methylamino)ethoxy]ethan-1- | | 4.31 (m, 5H), 4.29-4.21 |
| eluting | | | | one; | | (m, 1H), 4.09-3.97 (m, |
| isomer) | | | | (2R)-2-(3-chloro-4,5- | | 6H), 3.52-3.47 (m, |
| | | | | difluorophenyl)-1-[5-(2,3-dihydro- | | 1H), 3.44-3.39 (m, |
| | | | | 1,4-benzodioxine-6-sulfonyl)- | | 1H), 2.69-2.57 (m, |
| | | | | 1H,2H,3H,4H,5H,6H-pyrrolo[3,4- | | 2H), 2.24 (s, 3H), 2.10 |
| | | | | c]pyrrol-2-yl]-2-[2- | | (s, 1H). |
| | | | | (methylamino)ethoxy]ethan-1-one | | |
| 99-12 | +++ | 2-2 | F | (3R)-3-[5-(2,3-dihydro-1,4- | 482.0 | (DMSO-d₆, 400 MHz) |
| (1^{st} | | | | benzodioxine-6-sulfonyl)- | | δ (ppm): 7.40-7.31 (m, |
| eluting | | | | 1H,2H,3H,4H,5H,6H-pyrrolo[3,4- | | 2H), 7.29-7.19 (m, |
| isomer) | | | | c]pyrrole-2-carbonyl]-3- | | 5H), 7.06-7.04 (m, |
| 99-13 | ++ | | | phenylpyrrolidine; | | 1H), 4.33-4.29 (m, |
| (2^{nd} | | | | (3S)-3-[5-(2,3-dihydro-1,4- | | 4H), 4.07-3.98 (m, |
| eluting | | | | benzodioxine-6-sulfonyl)- | | 4H), 3.86-3.80 (m, |
| isomer) | | | | 1H,2H,3H,4H,5H,6H-pyrrolo[3,4- | | 2H), 3.73-3.47 (m, |
| | | | | c]pyrrole-2-carbonyl]-3- | | 4H), 2.93-2.83 (m, |
| | | | | phenylpyrrolidine | | 2H), 2.34-2.27 (m, |
| | | | | | | 1H), 2.19-2.12 (m, |
| | | | | | | 1H). |
| 99-14 | ++++ | 2-2 | F | (2R)-3-amino-1-[5-(2,3-dihydro- | 456.3 | (DMSO-d₆, 300 MHz,) |
| | | | | 1,4-benzodioxine-6-sulfonyl)- | | δ (ppm): 7.33-7.23 (m, |
| | | | | 1H,2H,3H,4H,5H,6H-pyrrolo[3,4- | | 7H), 7.05 (d, J = 8.1, |
| | | | | c]pyrrol-2-yl]-2-phenylpropan-1- | | Hz, 1H), 4.45-4.30 (m, |
| | | | | one; or | | 5H), 4.05-3.90 (m, |
| | | | | (2S)-3-amino-1-[5-(2,3-dihydro- | | 6H), 3.87-3.76 (m, |
| | | | | 1,4-benzodioxine-6-sulfonyl)- | | 1H), 3.71-3.68 (m, |
| | | | | 1H,2H,3H,4H,5H,6H-pyrrolo[3,4- | | 1H), 3.10-3.00 (m, |
| | | | | c]pyrrol-2-yl]-2-phenylpropan-1- | | 1H), 2.67-2.36 (m, |
| | | | | one | | 1H). |
| 99-15 | ++ | 2-2 | F | (2S)-2-amino-1-[5-(2,3-dihydro- | 442.1 | (CD₃OD, 300 MHz) δ |
| | | | | 1,4-benzodioxine-6-sulfonyl)- | | (ppm): 7.49-7.40 (m, |
| | | | | 1H,2H,3H,4H,5H,6H-pyrrolo[3,4- | | 5H), 7.32-7.28 (m, |
| | | | | c]pyrrol-2-yl]-2-phenylethan-1- | | 2H), 7.01-6.98 (m, |
| | | | | one; or | | 1H), 5.16 (s, 1H), 4.43- |
| | | | | (2R)-2-amino-1-[5-(2,3-dihydro- | | 4.25 (m, 6H), 4.14-3.94 |
| | | | | 1,4-benzodioxine-6-sulfonyl)- | | (m, 5H), 3.63-3.58 (m, |
| | | | | 1H,2H,3H,4H,5H,6H-pyrrolo[3,4- | | 1H). |
| | | | | c]pyrrol-2-yl]-2-phenylethan-1- | | |
| | | | | one | | |
| 99-16 | −− | 2-2, 82 | F | N-[(1R)-2-[5-(2,3-dihydro-1,4- | 541.0 | (DMSO-d₆, 400 MHz) δ |
| (1^{st} | | | | benzodioxine-6-sulfonyl)- | | (ppm): 8.31-8.30 (m, |
| eluting | | | | 1H,2H,3H,4H,5H,6H-pyrrolo[3,4- | | 1H), 7.28-7.23 (m, |
| isomer) | | | | c]pyrrol-2-yl]-1-(3,4-dihydro-2H- | | 2H), 7.07-7.05 (m, |
| 99-17 | ++ | | | 1,4-benzoxazin-6-yl)-2- | | 1H), 6.58-6.55 (m, |
| (2^{nd} | | | | oxoethyl]acetamide; | | 2H), 6.44-6.43 (m, |
| eluting | | | | N-[(1S)-2-[5-(2,3-dihydro-1,4- | | 1H), 5.84 (s, 1H), 5.27- |
| isomer) | | | | benzodioxine-6-sulfonyl)- | | 5.24 (m, 1H), 4.33-4.30 |
| | | | | 1H,2H,3H,4H,5H,6H-pyrrolo[3,4- | | (m, 5H), 4.08-4.00 (m, |
| | | | | c]pyrrol-2-yl]-1-(3,4-dihydro-2H- | | 9H), 3.23 (s, 2H), 1.82 |
| | | | | 1,4-benzoxazin-6-yl)-2- | | (s, 3H). |
| | | | | oxoethyl]acetamide | | |
| 99-18 | +++ | 2-2, 79 | F | (2S)-1-[5-(2,3-dihydro-1,4- | 484.4 | (CD₃OD, 400 MHz) δ |
| (1^{st} | | | | benzodioxine-6-sulfonyl)- | | (ppm): 7.30-7.21 (m, |
| eluting | | | | 1H,2H,3H,4H,5H,6H-pyrrolo[3,4- | | 5H), 7.00-6.98 (m, |
| isomer) | | | | c]pyrrol-2-yl]-2-(2,3-dihydro-1H- | | 1H), 5.24-5.22 (m, |
| | | | | isoindol-4-yl)-2-hydroxyethan-1- | | 1H), 4.72-4.63 (m, |
| | | | | one; or | | 1H), 4.38-4.23 (m, |
| | | | | (2R)-1-[5-(2,3-dihydro-1,4- | | 9H), 4.07-3.97 (m, |
| | | | | benzodioxine-6-sulfonyl)- | | 5H), 3.70-3.67 (m, |
| | | | | 1H,2H,3H,4H,5H,6H-pyrrolo[3,4- | | 1H). |
| | | | | c]pyrrol-2-yl]-2-(2,3-dihydro-1H- | | |
| | | | | isoindol-4-yl)-2-hydroxyethan-1- | | |
| | | | | one | | |
| 100-1 | ++++ | 19-1, 7-3, | G | (2S)-1-[5-(2,3-dihydro-1,4- | 512.4 | (DMSO-d₆, 400 MHz,) |
| (1^{st} | | 2-2 | | benzodioxine-6-sulfonyl)- | | δ (ppm): 7.42-7.24 (m, |
| eluting | | | | 1H,2H,3H,4H,5H,6H-pyrrolo[3,4- | | 2H), 7.12-6.99 (m, |
| isomer) | | | | c]pyrrol-2-yl]-2-hydroxy-2-(2- | | 4H), 5.37-5.30 (m, |
| 100-2 | + | | | methyl-1,2,3,4- | | 1H), 5.29-5.23 (m, |
| (2^{nd} | | | | tetrahydroisoquinolin-5-yl)ethan- | | 1H), 4.42-4.18 (m, |
| eluting | | | | 1-one; | | 5H), 4.14-3.89 (m, |
| isomer) | | | | (2R)-1-[5-(2,3-dihydro-1,4- | | 6H), 3.67-3.47 (m, |
| | | | | benzodioxine-6-sulfonyl)- | | 3H), 3.08-2.86 (s, 2H), |
| | | | | 1H,2H,3H,4H,5H,6H-pyrrolo[3,4- | | 2.82-2.60 (m, 2H), |
| | | | | c]pyrrol-2-yl]-2-hydroxy-2-(2- | | 2.50-2.40 (s, 3H). |
| | | | | methyl-1,2,3,4- | | |
| | | | | tetrahydroisoquinolin-5-yl)ethan- | | |
| | | | | 1-one | | |
| 100-3 | ++++ | 19-1, 7-3, | G | (2R)-1-[5-(2,3-dihydro-1,4- | 539.4 | (DMSO-d₆, 400 MHz,) |
| (1^{st} | | 2-2 | | benzodioxine-6-sulfonyl)- | | δ (ppm): 7.91 (d, J = |
| eluting | | | | 1H,2H,3H,5H-pyrrolo[3,4- | | 8.0 Hz, 1H), 7.41-7.32 |
| isomer) | | | | c]pyrrol-2-yl]-2-(2-methyl-1,3- | | (m, 4H), 7.14-7.12 (m, |
| 100-4 | ++ | | | benzothiazol-4-yl)-3- | | 2H), 7.06-7.04 (m, |
| (2^{nd} | | | | (methylamino)propan-1-one; | | 1H), 5.00-4.98 (m, |
| eluting | | | | (2S)-1-[5-(2,3-dihydro-1,4- | | 1H), 4.77-4.73 (m, |
| isomer) | | | | benzodioxine-6-sulfonyl)- | | 1H), 4.40-4.26 (m, |
| | | | | 1H,2H,3H,5H-pyrrolo[3,4- | | 7H), 3.24-3.18 (m, |
| | | | | c]pyrrol-2-yl]-2-(2-methyl-1,3- | | 1H), 2.84 (s, 3H), 2.76- |
| | | | | benzothiazol-4-yl)-3- | | 2.72 (m, 1H), 2.29 (s, |
| | | | | (methylamino)propan-1-one | | 3H). |
| 100-5 | ++ | 2-2, 88, | G | (2S)-1-[5-(2,3-dihydro-1,4- | 528.0 | (DMSO-d₆, 400 MHz) |
| (1^{st} | | 7-7 | | benzodioxine-6-sulfonyl)- | | δ (ppm): 7.29-7.24 (m, |
| eluting | | | | 1H,2H,3H,4H,5H,6H-pyrrolo[3,4- | | 3H), 7.07-7.05 (m, |
| isomer) | | | | c]pyrrol-2-yl]-2-hydroxy-2-{3-[(1- | | 1H), 6.99-6.97 (m, |
| 100-6 | ++++ | | | methylazetidin-3- | | 1H), 6.85 (s, 1H), 6.77- |
| (2^{nd} | | | | yl)oxy]phenyl}ethan-1-one; | | 6.74 (m, 1H), 5.74 (br s, |
| eluting | | | | (2R)-1-[5-(2,3-dihydro-1,4- | | 1H), 5.15 (s, 1H), |
| isomer) | | | | benzodioxine-6-sulfonyl)- | | 4.88-4.84 (m,1H), |
| | | | | 1H,2H,3H,4H,5H,6H-pyrrolo[3,4- | | 4.32-4.11(m, 5H), |
| | | | | c]pyrrol-2-yl]-2-hydroxy-2-{3-[(1- | | 4.11-4.00 (m, 3H), |
| | | | | methylazetidin-3- | | 4.00-3.94 (m, 6H), |
| | | | | yl)oxy]phenyl}ethan-1-one | | 3.49-3.45 (m, 2H), 2.67 |
| | | | | | | (s, 3H). |
| 101-1 | +++ | 2-25, 2-2 | H | (2R)-1-[5-(2,3-dihydro-1,4- | 532.6 | (CDCl₃, 400 MHz) δ |
| (1^{st} | | | | benzodioxine-6-sulfonyl)- | | (ppm): 7.36-7.28 (m, |
| eluting | | | | 1H,2H,3H,4H,5H,6H-pyrrolo[3,4- | | 2H), 7.11-7.07 (m, |
| isomer) | | | | c]pyrrol-2-yl]-3-(dimethylamino)- | | 1H), 7.00-6.97 (m, |
| 101-2 | + | | | 2-(5-fluoro-2- | | 1H), 6.95-6.89 (m, |
| (2^{nd} | | | | methoxyphenyl)propan-1-one; | | 1H), 6.83-6.80 (m, |
| eluting | | | | (2S)-1-[5-(2,3-dihydro-1,4- | | 1H), 4.36-4.22 (m, |
| isomer) | | | | benzodioxine-6-sulfonyl)- | | 7H), 4.13-4.03 (m, |
| | | | | 1H,2H,3H,4H,5H,6H-pyrrolo[3,4- | | 5H), 3.93-3.89 (m, |
| | | | | c]pyrrol-2-yl]-3-(dimethylamino)- | | 1H), 3.86 (m, 3H), |
| | | | | 2-(5-fluoro-2- | | 3.28-3.26 (m, 1H), |
| | | | | methoxyphenyl)propan-1-one | | 2.33-2.23 (m, 7H). |
| 101-3 | ++ | 3-17, 2-2 | H | (2R)-1-[5-(2,3-dihydro-1,4- | 514.4 | (DMSO-d₆, 300 MHz) |
| (1^{st} | | | | benzodioxine-6-sulfonyl)- | | δ (ppm): 7.32-7.20 (m, |
| eluting | | | | 1H,2H,3H,4H,5H,6H-pyrrolo[3,4- | | 7H), 7.06-7.03 (m, |
| isomer) | | | | c]pyrrol-2-yl]-3-(3-fluoroazetidin- | | 1H), 5.16-4.93 (m, |
| 101-4 | −− | | | 1-yl)-2-phenylpropan-1-one; | | 1H), 4.41-4.28 (m, |
| (2^{nd} | | | | (2S)-1-[5-(2,3-dihydro-1,4- | | 5H), 4.04-3.93 (m, |
| eluting | | | | benzodioxine-6-sulfonyl)- | | 7H), 3.66-3.61 (m, |
| isomer) | | | | 1H,2H,3H,4H,5H,6H-pyrrolo[3,4- | | 1H), 3.53-3.42 (m, |
| | | | | c]pyrrol-2-yl]-3-(3-fluoroazetidin- | | 2H), 3.12-2.95 (m, |
| | | | | 1-yl)-2-phenylpropan-1-one | | 3H), 2.61-2.55 (m, |
| | | | | | | 1H). |
| 101-5 | ++ | 2-2, 3-17 | H | 1-[5-(2,3-dihydro-1,4- | 510.4 | (CD₃OD, 300 MHz) δ |
| | | | | benzodioxine-6-sulfonyl)- | | (ppm): 7.43-7.29 (m, |
| | | | | 1H,2H,3H,4H,5H,6H-pyrrolo[3,4- | | 7H), 7.02-6.99 (m, |
| | | | | c]pyrrol-2-yl]-2-phenyl-3- | | 1H), 4.41-4.29 (m, |
| | | | | (pyrrolidin-1-yl)propan-1-one | | 6H), 4.25-4.05 (m, |
| | | | | | | 6H), 3.81-3.74 (m, |
| | | | | | | 2H), 3.24-3.15 (m, |
| | | | | | | 5H), 2.03-1.94 (m, |
| | | | | | | 4H). |
| 101-6 | ++ | 2-2, 3-17 | H | 1-[5-(2,3-dihydro-1,4- | 526.4 | (CDCl₃, 300 MHz) δ |
| | | | | benzodioxine-6-sulfonyl)- | | (ppm): 7.48-7.01 (m, |
| | | | | 1H,2H,3H,4H,5H,6H-pyrrolo[3,4- | | 7H), 6.99-6.98 (m, |
| | | | | c]pyrrol-2-yl]-3-(morpholin-4-yl)- | | 1H), 4.49-4.25 (m, |
| | | | | 2-phenylpropan-1-one | | 7H), 4.18-3.82 (m, |
| | | | | | | 10H), 3.75-3.66 (m, |
| | | | | | | 1H), 3.49-3.26 (m, |
| | | | | | | 5H). |
| 102-1 | ++ | 2-2 | I, F | 1-[(1S)-2-[5-(2,3-dihydro-1,4- | 499.4 | (CD₃OD, 300 MHz) δ |
| | | | | benzodioxine-6-sulfonyl)- | | (ppm): 7.41-7.31 (m, |
| | | | | 1H,2H,3H,4H,5H,6H-pyrrolo[3,4- | | 7H), 7.01 (d, J = 9.3 |
| | | | | c]pyrrol-2-yl]-2-oxo-1- | | Hz, 1H), 5.46 (s, 1H), |
| | | | | phenylethyl]-3-methylurea; or | | 4.49-4.44 (m, 1H), |
| | | | | 1-[(1R)-2-[5-(2,3-dihydro-1,4- | | 4.33-4.30 (m, 4H), |
| | | | | benzodioxine-6-sulfonyl)- | | 4.25-4.19 (m, 1H), |
| | | | | 1H,2H,3H,4H,5H,6H-pyrrolo[3,4- | | 4.10-4.04 (m, 5H), |
| | | | | c]pyrrol-2-yl]-2-oxo-1- | | 3.92-3.88 (m, 1H), 2.69 |
| | | | | phenylethyl]-3-methylurea | | (s, 3H). |
| 102-2 | +++ | 2-2, 16-1 | I | N-[(1S)-1-(3-chlorophenyl)-2-[5- | 518.2 | (DMSO-d₆, 300 MHz,) |
| | | | | (2,3-dihydro-1,4-benzodioxine-6- | | δ (ppm): 8.63-8.61 (m, |
| | | | | sulfonyl)-1H,2H,3H,4H,5H,6H- | | 1H), 7.47-7.21 (m, |
| | | | | pyrrolo[3,4-c]pyrrol-2-yl]-2- | | 6H), 7.06 (d, J = 8.4 |
| | | | | oxoethyl]acetamide; or | | Hz, 1H), 5.56 (d, J = |
| | | | | N-[(1R)-1-(3-chlorophenyl)-2-]5- | | 7.8 Hz, 1H), 4.47-4.21 |
| | | | | (2,3-dihydro-1,4-benzodioxine-6- | | (m, 5H), 4.16-3.91 (m, |
| | | | | sulfonyl)-1H,2H,3H,4H,5H,6H- | | 7H), 1.86 (s, 3H). |
| | | | | pyrrolo[3,4-c]pyrrol-2-yl]-2- | | |
| | | | | oxoethyl]acetamide | | |
| 103-1 | ++ | 2-2, 34-1 | J | (2R)-1-[5-(2,3-dihydro-1,4- | 512.4 | (DMSO-d₆, 300 MHz) |
| (1^{st} | | | | benzodioxine-6-sulfonyl)- | | δ (ppm): 7.29-7.23 (m, |
| eluting | | | | 1H,2H,3H,4H,5H,6H-pyrrolo[3,4- | | 7H), 7.07-7.04 (d, J = |
| isomer) | | | | c]pyrrol-2-yl]-3-[(oxetan-3- | | 8.4 Hz, 1H), 4.56-4.53 |
| 103-2 | + | | | yl)amino]-2-phenylpropan-1-one; | | (m, 2H), 4.32-4.28 (m, |
| (2^{nd} | | | | (2S)-1-[5-(2,3-dihydro-1,4- | | 4H), 4.00-3.89 (m, |
| eluting | | | | benzodioxine-6-sulfonyl)- | | 8H), 3.20-3.14 (m, |
| isomer) | | | | 1H,2H,3H,4H,5H,6H-pyrrolo[3,4- | | 2H), 3.06-3.03 (m, |
| | | | | c]pyrrol-2-yl]-3-[(oxetan-3- | | 2H), 2.19-2.15 (m, |
| | | | | yl)amino]-2-phenylpropan-1-one | | 1H), 1.39-1.29 (m, |
| | | | | | | 2H). |
| 103-3 | +++ | 2-2, 35-2 | J | (2R)-3-(azetidin-1-yl)-1-[5-(2,3- | 496.4 | (DMSO-d₆, 300 MHz) |
| (1^{st} | | | | dihydro-1,4-benzodioxine-6- | | δ (ppm): 7.28-7.24 (m, |
| eluting | | | | sulfonyl)-1H,2H,3H,4H,5H,6H- | | 7H), 7.07-7.04 (d, J = |
| isomer) | | | | | | 8.4 Hz, 1H), 4.31-4.30 |
| 103-4 | + | | | pyrrolo[3,4-c]pyrrol-2-yl]-2- | | (m, 5H), 4.05-3.93 (m, |
| (2^{nd} | | | | phenylpropan-1-one; | | 7H), 3.62 (s, 1H), 3.17- |
| eluting | | | | (2S)-3-(azetidin-1-yl)-1-[5-(2,3- | | 3.11 (m, 4H), 2.50-2.49 |
| isomer) | | | | dihydro-1,4-benzodioxine-6- | | (m, 2H), 1.91-1.87 (m, |
| | | | | sulfonyl)-1H,2H,3H,4H,5H,6H- | | 2H). |
| | | | | pyrrolo[3,4-c]pyrrol-2-yl]-2- | | |
| | | | | phenylpropan-1-one | | |
| 103-5 | +++ | 35-1, 2-2 | J | (2R)-1-[5-(2,3-dihydro-1,4- | 512.5 | (DMSO-d₆, 400 MHz) |
| (1^{st} | | | | benzodioxine-6-sulfonyl)- | | δ (ppm): 7.27-7.24 (m, |
| eluting | | | | 1H,2H,3H,4H,5H,6H-pyrrolo[3,4- | | 7H), 7.05 (d, J = 8.4 |
| isomer) | | | | c]pyrrol-2-yl]-3-(3- | | Hz, 1H), 5.21 (d, J = |
| 103-6 | + | | | hydroxyazetidin-1-yl)-2- | | 6.0 Hz, 1H), 4.39-4.30 |
| (2^{nd} | | | | phenylpropan-1-one; | | (m, 5H), 4.07-3.88 (m, |
| eluting | | | | (2S)-1-[5-(2,3-dihydro-1,4- | | 8H), 3.62-3.58 (m, |
| isomer) | | | | benzodioxine-6-sulfonyl)- | | 1H), 3.41-3.39 (m, |
| | | | | 1H,2H,3H,4H,5H,6H-pyrrolo[3,4- | | 2H), 3.07-3.02 (m, |
| | | | | c]pyrrol-2-yl]-3-(3- | | 1H), 2.72-2.62 (m, |
| | | | | hydroxyazetidin-1-yl)-2- | | 2H), 2.50-2.49 (m, |
| | | | | phenylpropan-1-one | | 1H). |
| 103-7 | ++++ | 2-2, 2-27, | J | (2S)-3-(azetidin-1-yl)-1-[5-(2,3- | 567.4 | (DMSO-d₆, 400 MHz) |
| (1^{st} | | 80 | | dihydro-1,4-benzodioxine-6- | | δ (ppm): 7.89-7.87 (m, |
| eluting | | | | sulfonyl)-1H,2H,3H,4H,5H,6H- | | 1H), 7.38-7.36 (m, |
| isomer) | | | | pyrrolo[3,4-c]pyrrol-2-yl]-2-(2- | | 1H), 7.33-7.25 (m, |
| 103-8 | ++ | | | methyl-1,3-benzothiazol-4- | | 2H), 7.24-7.22 (m, |
| (2^{nd} | | | | yl)propan-1-one; | | 1H), 7.05 (d, J = 8.4 |
| eluting | | | | (2R)-3-(azetidin-1-yl)-1-[5-(2,3- | | Hz, 1H), 4.68-4.64 (m, |
| isomer) | | | | dihydro-1,4-benzodioxine-6- | | 1H), 4.42-4.23 (m, |
| | | | | sulfonyl)-1H,2H,3H,4H,5H,6H- | | 5H), 4.17-4.15 (m, |
| | | | | pyrrolo[3,4-c]pyrrol-2-yl]-2-(2- | | 1H), 4.06-3.88 (m, 6H), |
| | | | | methyl-1,3-benzothiazol-4- | | 3.15-3.05 (m, 5H), |
| | | | | yl)propan-1-one | | 2.81 (s, 3H), 2.56-2.51 |
| | | | | | | (m, 1H), 1.91-1.84 (m, |
| | | | | | | 2H). |
| 103-9 | ++ | 22-2B, | J | (2S)-1-(5-{2H,3H- | 476.3 | (DMSO-d₆, 400 MHz) |
| (1^{st} | | 38-1, 39-1, | | [1,4]dioxino[2,3-b]pyridine-7- | | δ (ppm): 8.16 (d, J = |
| eluting | | 40-1 | | sulfonyl}-1H,2H,3H,4H,5H,6H- | | 2.4 Hz, 1H), 7.62 (d, |
| isomer) | | | | pyrrolo[3,4-c]pyrrol-2-yl)-2-(2- | | J = 2.0 Hz, 1H), 7.19- |
| 103-10 | + | | | fluoro-5-methylphenyl)-2- | | 7.15 (m, 1H), 7.14-7.12 |
| (2^{nd} | | | | hydroxyethan-1-one; | | (m, 1H), 7.06-7.02 (m, |
| eluting | | | | (2R)-1-(5-{2H,3H- | | 1H), 5.69 (d, J = 6.4 |
| isomer) | | | | [1,4]dioxino[2,3-b]pyridine-7- | | Hz, 1H), 5.36 (d, J = |
| | | | | sulfonyl}-1H,2H,3H,4H,5H,6H- | | 6.8 Hz, 1H), 4.52-4.50 |
| | | | | pyrrolo[3,4-c]pyrrol-2-yl)-2-(2- | | (m, 2H), 4.38-4.32 (m, |
| | | | | fluoro-5-methylphenyl)-2- | | 3H), 4.11-4.02 (m, |
| | | | | hydroxyethan-1-one | | 6H), 3.99-3.85 (m, |
| | | | | | | 1H), 2.26 (s, 3H). |
| 103-11 | ++ | 42-1, 2-2 | J | (2S)-2-[3- | 567.4 | (CDCl₃, 400 MHz) δ |
| (1^{st} | | | | (difluoromethoxy)phenyl]-1-[5- | | (ppm): 7.37-7.25 (m, |
| eluting | | | | (2,3-dihydro-1,4-benzodioxine-6- | | 5H), 7.10-7.07 (m, |
| isomer) | | | | sulfonyl)-1H,2H,3H,4H,5H,6H- | | 1H), 6.99-6.96 (m, |
| 103-12 | +++ | | | pyrrolo[3,4-c]pyrrol-2-yl]-2-(2- | | 1H), 6.73-6.35 (m, |
| (2^{nd} | | | | methoxyethoxy)ethan-1-one; | | 1H), 5.11 (s, 1H), 4.35- |
| eluting | | | | (2R)-2-[3- | | 4.31 (m, 5H), 4.30-4.17 |
| isomer) | | | | (difluoromethoxy)phenyl]-1-[5- | | (m, 2H), 4.13-4.02 (m, |
| | | | | (2,3-dihydro-1,4-benzodioxine-6- | | 5H), 3.76-3.58 (m, |
| | | | | sulfonyl)-1H,2H,3H,4H,5H,6H- | | 4H), 3.38 (s, 3H). |
| | | | | pyrrolo[3,4-c]pyrrol-2-yl]-2-(2- | | |
| | | | | methoxyethoxy)ethan-1-one | | |
| 103-17 | +++ | 2-2, 81 | J | (2R)-1-[5-(2,3-dihydro-1,4- | 483.2 | (DMSO-d₆, 400 MHz) |
| (1^{st} | | | | benzodioxine-6-sulfonyl)- | | δ (ppm): 12.90 (s, 1H), |
| eluting | | | | 1H,2H,3H,4H,5H,6H-pyrrolo[3,4- | | 7.79 (d, J = 8.0 Hz, |
| isomer) | | | | c]pyrrol-2-yl]-2-hydroxy-2-(1H- | | 1H), 7.50-7.43 (m, |
| 103-18 | ++ | | | indazol-3-yl)ethan-1-one; | | 1H), 7.33-7.30 (m, |
| (2^{nd} | | | | (2S)-1-[5-(2,3-dihydro-1,4- | | 1H), 7.27-7.18 (m, |
| eluting | | | | benzodioxine-6-sulfonyl)- | | 2H), 7.10-7.00 (m, |
| isomer) | | | | 1H,2H,3H,4H,5H,6H-pyrrolo[3,4- | | 2H), 5.54 (br s, 2H), |
| | | | | c]pyrrol-2-yl]-2-hydroxy-2-(1H- | | 4.45-4.24 (m, 5H), |
| | | | | indazol-3-yl)ethan-1-one | | 4.14-4.09 (m, 1H), |
| | | | | | | 4.04-3.95 (m, 5H), |
| | | | | | | 3.76-3.73 (m, 1H). |
| 103-19 | + | 72 | J | (2S)-1-[2-(2,3-dihydro-1,4- | 441.9 | (CD₃OD, 300 MHz) δ |
| | | | | benzodioxine-6-sulfonyl)- | | (ppm): 8.06-7.99 (m, |
| | | | | 2H,4H,5H,6H-pyrrolo[3,4- | | 1H), 7.48-7.32 (m, |
| | | | | c]pyrazol-5-yl]-2-hydroxy-2- | | 7H), 7.01-6.99 (m, |
| | | | | phenylethan-1-one | | 1H), 5.35 (s, 1H), 4.71- |
| | | | | | | 4.23 (m, 8H). |
| 103-20 | +++ | 59-1, 54-6, | J | (2S)-2-(2,3-dihydro-1-benzofuran- | 486.3 | (DMSO-d₆, 300 MHz) |
| (1^{st} | | 39-1 | | 4-yl)-1-(5-{2H,3H- | | δ (ppm): 8.15 (d, J = |
| eluting | | | | [1,4]dioxino[2,3-b]pyridine-7- | | 2.1 Hz, 1H), 7.61 (d, |
| isomer) | | | | sulfonyl}-1H,2H,3H,4H,5H,6H- | | J = 2.1 Hz, 1H), 7.03- |
| 103-21 | + | | | pyrrolo[3,4-c]pyrrol-2-yl)-2- | | 7.08 (m, 1H), 6.77 (d, |
| (2^{nd} | | | | hydroxyethan-1-one; | | J = 7.5 Hz, 1H), 6.69 (d, |
| eluting | | | | (2R)-2-(2,3-dihydro-1-benzofuran- | | J = 8.1 Hz, 1H), 5.47 |
| isomer) | | | | 4-yl)-1-(5-{2H,3H- | | (d, J = 5.7 Hz, 1H), |
| | | | | [1,4]dioxino[2,3-b]pyridine-7- | | 5.14 (d, J = 5.7 Hz, |
| | | | | sulfonyl}-1H,2H,3H,4H,5H,6H- | | 1H), 4.52-4.46 (m, |
| | | | | pyrrolo[3,4-c]pyrrol-2-yl)-2- | | 4H), 4.35-4.24 (m, |
| | | | | hydroxyethan-1-one | | 3H), 4.14-3.99 (m, |
| | | | | | | 6H), 3.78-3.73 (m, |
| | | | | | | 1H), 3.24-3.07 (m, 2H). |
| 103-22 | ++ | 2-2, 56-1 | J | (2R)-1-[5-(2,3-dihydro-1,4- | 514.2 | (CDCl₃, 400 MHz) δ |
| (1^{st} | | | | benzodioxine-6-sulfonyl)- | | (ppm): 7.37-7.27 (m, |
| eluting | | | | 1H,2H,3H,4H,5H,6H-pyrrolo[3,4- | | 2H), 7.02-6.96 (m, |
| isomer) | | | | c]pyrrol-2-yl]-2-hydroxy-2-(4- | | 1H), 6.73 (d, J = 7.6 |
| 103-23 | ++++ | | | methyl-3,4-dihydro-2H-1,4- | | Hz, 1H), 6.63-6.61 (m, |
| (2^{nd} | | | | benzoxazin-6-yl)ethan-1-one; | | 2H), 4.89-4.87 (m, |
| eluting | | | | (2S)-1-[5-(2,3-dihydro-1,4- | | 1H), 4.33-4.25 (m, |
| isomer) | | | | benzodioxine-6-sulfonyl)- | | 7H), 4.15-4.01 (m, |
| | | | | 1H,2H,3H,4H,5H,6H-pyrrolo[3,4- | | 6H), 3.73-3.71 (m, |
| | | | | c]pyrrol-2-yl]-2-hydroxy-2-(4- | | 1H), 3.29-3.27 (m, |
| | | | | methyl-3,4-dihydro-2H-1,4- | | 2H), 2.90 (s, 3H). |
| | | | | benzoxazin-6-yl)ethan-1-one | | |
| 103-24 | +++ | 60-1, 43-1, | J | N-[(1S)-2-[5-(2,3-dihydro-1,4- | 539.3 | (CD₃OD, 400 MHz) δ |
| (l^{sl} | | 2-2 | | benzodioxine-6-sulfonyl)- | | (ppm): 7.55-7.53 (m, |
| eluting | | | | 1H,2H,3H,4H,5H,6H-pyrrolo[3,4- | | 1H), 7.42-7.26 (m, |
| isomer) | | | | c]pyrrol-2-yl]-1-(2-methyl-1,3- | | 4H), 6.99-6.98 (m, |
| 103-25 | + | | | benzoxazol-4-yl)-2- | | 1H), 6.15 (s, 1H), 4.53- |
| (2^{nd} | | | | oxoethyl]acetamide; | | 4.51 (m, 1H), 4.33-4.29 |
| eluting | | | | N-[(1R)-2-[5-(2,3-dihydro-1,4- | | (m, 4H), 4.23-4.20 (m, |
| isomer) | | | | benzodioxine-6-sulfonyl)- | | 1H), 4.09-3.91 (m, |
| | | | | 1H,2H,3H,4H,5H,6H-pyrrolo[3,4- | | 6H), 2.63 (s, 3H), 1.99 |
| | | | | c]pyrrol-2-yl]-1-(2-methyl-1,3- | | (s, 3H). |
| | | | | benzoxazol-4-yl)-2- | | |
| | | | | oxoethyl]acetamide | | |
| 103-26 | +++ | 55-1, 43-1, | J | (2S)-1-{5-[(8-fluoro-2,3-dihydro- | 516.2 | (CD₃OD, 300 MHz) δ |
| (1^{st} | | 2-2 | | 1,4-benzodioxin-6-yl)sulfonyl]- | | (ppm): 7.54-7.51 (m, |
| eluting | | | | 1H,2H,3H,4H,5H,6H-pyrrolo[3,4- | | 1H), 7.44-7.29 (m, |
| isomer) | | | | c]pyrrol-2-yl}-2-hydroxy-2-(2- | | 2H), 7.25-7.12 (m, |
| 103-27 | + | | | methyl-1,3-benzoxazol-4-yl)ethan- | | 2H), 5.85 (s, 1H), 4.41- |
| (2^{nd} | | | | 1-one; | | 4.30 (m, 6H), 4.16-3.81 |
| eluting | | | | (2R)-1-{5-[(8-fluoro-2,3-dihydro- | | (m, 6H), 2.64 (s, 3H). |
| isomer) | | | | 1,4-benzodioxin-6-yl)sulfonyl]- | | |
| | | | | 1H,2H,3H,4H,5H,6H-pyrrolo[3,4- | | |
| | | | | c]pyrrol-2-yl}-2-hydroxy-2-(2- | | |
| | | | | methyl-1,3-benzoxazol-4-yl)ethan- | | |
| | | | | 1-one | | |
| 103-28 | ++ | 54-7, 61-1, | J | (2S)-1-[5-(2,3-dihydro-1,4- | 498.3 | (DMSO-d₆, 400 MHz) |
| (1^{st} | | 2-2 | | benzodioxine-6-sulfonyl)- | | δ 7.79 (d, J = 8.0 Hz, |
| eluting | | | | 1H,2H,3H,4H,5H,6H-pyrrolo[3,4- | | 1H), 7.64 (s, 1H), 7.36- |
| isomer) | | | | c]pyrrol-2-yl]-2-hydroxy-2-(3- | | 7.32 (m, 1H), 7.27- |
| 103-29 | + | | | methyl-1,2-benzoxazol-4-yl)ethan- | | 7.20 (m, 2H), 7.03 (d, |
| (2^{nd} | | | | 1-one; | | J = 8.4 Hz, 1H), 6.00 (d, |
| eluting | | | | (2R)-1-[5-(2,3-dihydro-1,4- | | J = 6.0 Hz, 1H), 5.35 |
| isomer) | | | | benzodioxine-6-sulfonyl)- | | (d, J = 5.6 Hz, 1H), |
| | | | | 1H,2H,3H,4H,5H,6H-pyrrolo[3,4- | | 4.30-4.20 (m, 5H), |
| | | | | c]pyrrol-2-yl]-2-hydroxy-2-(3- | | 4.06-3.99 (m, 7H), 2.52 |
| | | | | methyl-1,2-benzoxazol-4-yl)ethan- | | (s, 3H). |
| | | | | 1-one | | |
| 103-30 | ++ | 50-2, 39-1 | J | (2S)-2-(1,2-benzoxazol-3-yl)-1-(5- | 485.3 | (DMSO-d₆, 400 MHz) |
| (1^{st} | | | | {2H,3H-[1,4]dioxino[2,3- | | δ (ppm): 8.14 (d, J = |
| eluting | | | | b]pyridine-7-sulfonyl}- | | 2.0 Hz. 1H), 7.90 (d, |
| isomer) | | | | 1H,2H,3H,4H,5H,6H-pyrrolo[3,4- | | J = 8.0 Hz, 1H), 7.72 (d, |
| 103-31 | + | | | c]pyrrol-2-yl)-2-hydroxyethan-1- | | J = 8.4 Hz, 1H), 7.68- |
| (2^{nd} | | | | one; | | 7.58 (m, 2H), 7.36 (t, |
| eluting | | | | (2R)-2-(1,2-benzoxazol-3-yl)-1- | | J = 7.6 Hz, 1H), 6.31 (d, |
| isomer) | | | | (5-{2H,3H-[1,4]dioxino[2,3- | | J = 6.4 Hz, 1H), 5.72 |
| | | | | b]pyridine-7-sulfonyl}- | | (d, J = 6.4 Hz, 1H), |
| | | | | 1H,2H,3H,4H,5H,6H-pyrrolo[3,4- | | 4.52-4.40 (m, 3H), 4.31 |
| | | | | c]pyrrol-2-yl)-2-hydroxyethan-1- | | (t, J = 4.0 Hz, 2H), |
| | | | | one | | 4.09-3.94 (m, 7H). |
| 103-32 | + | 39-1, 54-3 | J | (2S)-2-(4-cyclopropyl-1-methyl- | 488.3 | (CD₃OD, 400 MHz) δ |
| (1^{st} | | | | 1H-pyrazol-3-yl)-1-(5-{2H,3H- | | (ppm): 8.19 (s, 1H), |
| eluting | | | | [1,4]dioxino[2,3-b]pyridine-7- | | 7.69 (s, 1H), 7.20 (s, |
| isomer) | | | | sulfonyl}-1H,2H,3H,4H,5H,6H- | | 1H), 5.33 (s, 1H), 4.59- |
| 103-33 | −− | | | pyrrolo[3,4-c]pyrrol-2-yl)-2- | | 4.52 (m, 2H), 4.39-4.01 |
| (2^{nd} | | | | hydroxyethan-1-one; | | (m, 9H), 3.77 (s, 3H), |
| eluting | | | | (2R)-2-(4-cyclopropyl-1-methyl- | | 3.70-3.58 (m, 1H), |
| isomer) | | | | 1H-pyrazol-3-yl)-1-(5-{2H,3H- | | 1.76-1.55 (m, 1H), |
| | | | | [1,4]dioxino[2,3-b]pyridine-7- | | 0.89-0.72 (m, 2H), |
| | | | | sulfonyl}-1H,2H,3H,4H,5H,6H- | | 0.57-0.49 (m, 1H), |
| | | | | pyrrolo[3,4-c]pyrrol-2-yl)-2- | | 0.42-0.31 (m, 1H). |
| | | | | hydroxyethan-1-one | | |
| 103-34 | + | 58-1, 39-1, | J | (2S)-1-(5-{2H,3H- | 542.4 | (CDCl₃, 400 MHz) δ |
| (1^{st} | | 54-5 | | [1,4]dioxino[2,3-b]pyridine-7- | | (ppm): 8.32 (s, 1H), |
| eluting | | | | sulfonyl}-1H,2H,3H,4H,5H,6H- | | 7.62 (s, 1H), 7.40 (s, |
| isomer) | | | | pyrrolo[3,4-c]pyrrol-2-yl)-2-[5-(4- | | 2H), 7.20-7.10 (m, |
| 103-35 | −− | | | fluorophenyl)-1-methyl-1H- | | 2H), 6.33 (s, 1H), 5.26 |
| (2^{nd} | | | | pyrazol-3-yl]-2-hydroxyethan-1- | | (s, 1H), 4.55 (s, 2H), |
| eluting | | | | one; | | 4.44-4.08 (m, 10H), |
| isomer) | | | | (2R)-1-(5-{2H,3H- | | 3.86 (s, 3H). |
| | | | | [1,4]dioxino[2,3-b]pyridine-7- | | |
| | | | | sulfonyl}-1H,2H,3H,4H,5H,6H- | | |
| | | | | pyrrolo[3,4-c]pyrrol-2-yl)-2-[5-(4- | | |
| | | | | fluorophenyl)-1-methyl-1H- | | |
| | | | | pyrazol-3-yl]-2-hydroxyethan-1- | | |
| | | | | one | | |
| 103-36 | + | 39-1, 54-8, | J | (2S)-2-(5-cyclopropyl-1-methyl- | 488.3 | (CD₃OD, 300 MHz) δ |
| (1^{st} | | 58-2 | | 1H-pyrazol-3-yl)-1-(5-{2H,3H- | | (ppm): 8.15 (d, J = 2.1 |
| eluting | | | | [1,4]dioxino[2,3-b]pyridine-7- | | Hz, 1H), 7.64 (d, J = |
| isomer) | | | | sulfonyl}-1H,2H,3H,4H,5H,6H- | | 2.1 Hz, 1H), 5.85 (s, |
| 103-37 | −− | | | pyrrolo[3,4-c]pyrrol-2-yl)-2- | | 1H), 5.14 (s, 1H), 4.60- |
| (2^{nd} | | | | hydroxyethan-1-one; | | 4.45 (m, 2H), 4.36-4.17 |
| eluting | | | | (2R)-2-(5-cyclopropyl-1-methyl- | | (m, 2H), 4.14-3.88 (m, |
| isomer) | | | | 1H-pyrazol-3-yl)-1-(5-{2H,3H- | | 7H), 3.96-3.91 (m, |
| | | | | [1,4]dioxino[2,3-b]pyridine-7- | | 1H), 3.80 (s, 3H), 1.82- |
| | | | | sulfonyl}-1H,2H,3H,4H,5H,6H- | | 1.70 (m, 1H), 1.02-0.86 |
| | | | | pyrrolo[3,4-c]pyrrol-2-yl)-2- | | (m, 2H), 0.65-0.53 (m, |
| | | | | hydroxyethan-1-one | | 2H). |
| 103-38 | + | 54-4, 39-1 | J | (2S)-1-(5-{2H,3H- | 484.3 | (CD₃OD, 400 MHz) δ |
| (1^{st} | | | | [1,4]dioxino[2,3-b]pyridine-7- | | (ppm): 8.45 (d, J = 6.8 |
| eluting | | | | sulfonyl}-1H,2H,3H,4H,5H,6H- | | Hz, 1H), 8.18 (d, J = |
| isomer) | | | | pyrrolo[3,4-c]pyrrol-2-yl)-2- | | 2.0 Hz, 1H), 7.94-7.89 |
| 103-39 | −− | | | hydroxy-2-{imidazo[1,2- | | (m, 1H), 7.67 (d, J = |
| (2^{nd} | | | | a]pyridin-8-yl}ethan-1-one; | | 2.0 Hz, 1H), 7.61 (s, |
| eluting | | | | (2R)-1-(5-{2H,3H- | | 1H), 7.43 (d, J = 7.2 |
| isomer) | | | | [1,4]dioxino[2,3-b]pyridine-7- | | Hz, 1H), 6.99 (d, J = |
| | | | | sulfonyl}-1H,2H,3H,4H,5H,6H- | | 6.8 Hz, 1H), 5.90 (s, |
| | | | | pyrrolo[3,4-c]pyrrol-2-yl)-2- | | 1H), 4.56-4.49 (m, |
| | | | | hydroxy-2-{imidazo[1,2- | | 3H), 4.37-4.30 (m, |
| | | | | a]pyridin-8-yl}ethan-1-one | | 2H), 4.20-4.10 (m, |
| | | | | | | 7H). |
| 103-40 | − | 46-1, 39-1, | J | (2S)-1-(5-{2H,3H- | 542.3 | (CDCl₃, 400 MHz) δ |
| (1^{st} | | 58-1 | | [1,4]dioxino[2,3-b]pyridine-7- | | (ppm): 8.28 (s, 1H), |
| eluting | | | | sulfonyl}-1H,2H,3H,4H,5H,6H- | | 7.59 (s, 1H), 7.55-7.41 |
| isomer) | | | | pyrrolo[3,4-c]pyrrol-2-yl)-2-[4-(4- | | (m, 3H), 7.08-7.06 (m, |
| 103-41 | + | | | fluorophenyl)-1-methyl-1H- | | 2H), 5.15 (s, 1H), 4.56- |
| (2^{nd} | | | | pyrazol-3-yl]-2-hydroxyethan-1- | | 4.52 (m, 2H), 4.41-4.00 |
| eluting | | | | one; | | (m, 8H), 3.94 (s, 3H), |
| isomer) | | | | (2R)-1-(5-{2H,3H- | | 3.86-3.83 (m, 1H), |
| | | | | [1,4]dioxino[2,3-b]pyridine-7- | | 3.47-3.44 (m, 1H). |
| | | | | sulfonyl}-1H,2H,3H,4H,5H,6H- | | |
| | | | | pyrrolo[3,4-c]pyrrol-2-yl)-2-[4-(4- | | |
| | | | | fluorophenyl)-1-methyl-1H- | | |
| | | | | pyrazol-3-yl]-2-hydroxyethan-1- | | |
| | | | | one | | |
| 103-42 | ++ | 2-2 | J | (2R)-1-[5-(2,3-dihydro-1,4- | 457.0 | (DMSO-d₆, 400 MHz) |
| | | | | benzodioxine-6-sulfonyl)- | | δ (ppm): 7.38-7.20 (m, |
| | | | | 1H,2H,3H,4H,5H,6H-pyrrolo[3,4- | | 7H), 7.06-7.03 (m, |
| | | | | c]pyrrol-2-yl]-2-hydroxy-2- | | 1H), 6.10 (s, 1H), 4.47- |
| | | | | phenylpropan-1-one; or (2S)-1-[5- | | 4.30 (m, 5H), 4.07-3.87 |
| | | | | (2,3-dihydro-1,4-benzodioxine-6- | | (m, 6H), 3.44-3.38 (m, |
| | | | | sulfonyl)-1H,2H,3H,4H,5H,6H- | | 1H). |
| | | | | pyrrolo[3,4-c]pyrrol-2-yl]-2- | | |
| | | | | hydroxy-2-phenylpropan-1-one | | |
| 103-43 | ++ | 2-2 | J | (2S)-1-[5-(2,3-dihydro-1,4- | 441.1 | (DMSO-d₆, 300 MHz) |
| | | | | benzodioxine-6-sulfonyl)- | | δ (ppm): 7.33-7.25 (m, |
| | | | | 1H,2H,3H,4H,5H,6H-pyrrolo[3,4- | | 7H), 7.07-7.04 (m, |
| | | | | c]pyrrol-2-yl]-2-phenylpropan-1- | | 1H), 4.39-4.21 (m, |
| | | | | one; or (2R)-1-[5-(2,3-dihydro- | | 5H), 3.99-3.91 (m, |
| | | | | 1,4-benzodioxine-6-sulfonyl)- | | 6H), 3.82-3.78 (m, |
| | | | | 1H,2H,3H,4H,5H,6H-pyrrolo[3,4- | | 2H), 1.28 (d, J = 6.6 |
| | | | | c]pyrrol-2-yl]-2-phenylpropan-1- | | Hz, 3H). |
| | | | | one | | |
| 103-44 | ++ | 38-1, 43-1, | J | (2S)-1-{5-[(2,2,3,3-²H₄)-2H,3H- | 503.0 | (DMSO-d₆, 400 MHz) |
| (1^{st} | | 90 | | [1,4]dioxino[2,3-b]pyridine-7- | | δ (ppm): 8.13 (d, J = |
| eluting | | | | sulfonyl]-1H,2H,3H,4H,5H,6H- | | 2.2 Hz, 1H), 7.65-7.51 |
| isomer) | | | | pyrrolo[3,4-c]pyrrol-2-yl}-2- | | (m, 2H), 7.35-7.25 (m, |
| 103-45 | + | | | hydroxy-2-(2-methyl-1,3- | | 2H), 5.68 (s, 2H), 4.41- |
| (2^{nd} | | | | benzoxazol-4-yl)ethan-1-one; | | 4.30 (m, 1H), 4.14-3.95 |
| eluting | | | | (2R)-1-{5-[(2,2,3,3-²H₄)-2H,3H- | | (m, 6H), 3.94-3.85 (m, |
| isomer) | | | | [1,4]dioxino[2,3-b]pyridine-7- | | 1H), 2.60 (s, 3H). |
| | | | | sulfonyl]-1H,2H,3H,4H,5H,6H- | | |
| | | | | pyrrolo[3,4-c]pyrrol-2-yl}-2- | | |
| | | | | hydroxy-2-(2-methyl-1,3- | | |
| | | | | benzoxazol-4-yl)ethan-1-one | | |
| 103-46 | + | 38-1, 43-1, | J | (2S)-1-{5-[2,3-dihydro(2,2,3,3- | 502.0 | (DMSO-d₆, 400 MHz) |
| (1^{st} | | 89 | | ²H₄)-1,4-benzodioxine-6- | | δ (ppm): 7.58 (s, 1H), |
| eluting | | | | sulfonyl]-1H,2H,3H,4H,5H,6H- | | 7.37-7.21 (m, 4H), 7.06 |
| isomer) | | | | pyrrolo[3,4-c]pyrrol-2-yl}-2- | | (d, J = 8.4 Hz, 1H), |
| 103-47 | ++++ | | | hydroxy-2-(2-methyl-1,3- | | 5.71 (q, J = 6.8 Hz, |
| (2^{nd} | | | | benzoxazol-4-yl)ethan-1-one; | | 2H), 4.38-4.35 (m, |
| eluting | | | | (2R)-1-{5-[2,3-dihydro(2,2,3,3- | | 1H), 4.11-3.86 (m, |
| isomer) | | | | ²H₄)-1,4-benzodioxine-6- | | 7H), 2.62 (s, 3H). |
| | | | | sulfonyl]-1H,2H,3H,4H,5H,6H- | | |
| | | | | pyrrolo[3,4-c]pyrrol-2-yl}-2- | | |
| | | | | hydroxy-2-(2-methyl-1,3- | | |
| | | | | benzoxazol-4-yl)ethan-1-one | | |
| 104-1 | ++ | 2-2, 37-1, | K, F | (2S)-1-[5-(2,3-dihydro-1,4- | 542.2 | (DMSO-d₆, 300 MHz) |
| (1^{st} | | 36-1 | | benzodioxine-6-sulfonyl)- | | δ (ppm): 7.37-7.32 (m, |
| eluting | | | | 1H,2H,3H,4H,5H,6H-pyrrolo[3,4- | | 5H), 7.28-7.23 (m, |
| isomer) | | | | c]pyrrol-2-yl]-2-{[1-(2- | | 2H), 7.06-7.04 (m, |
| 104-2 | +++ | | | hydroxyethyl)azetidin-3-yl]oxy}- | | 1H), 5.00 (s, 1H), 4.34- |
| (2^{nd} | | | | 2-phenylethan-1-one; | | 4.29(m, 6H), 4.09-3.91 |
| eluting | | | | (2R)-1-[5-(2,3-dihydro-1,4- | | (m, 8H), 3.45-3.26 (m, |
| isomer) | | | | benzodioxine-6-sulfonyl)- | | 3H), 2.87-2.84 (m, |
| | | | | 1H,2H,3H,4H,5H,6H-pyrrolo[3,4- | | 1H), 2.73-2.70 (m, |
| | | | | c]pyrrol-2-yl]-2-{[1-(2- | | 1H), 2.50-2.38 (m, |
| | | | | hydroxyethyl)azetidin-3-yl]oxy}- | | 2H). |
| | | | | 2-phenylethan-1-one | | |
| 105-1 | + | | L | (2S)-1-[5-(2,3-dihydro-1,4- | 514.3 | (CD₃OD, 400 MHz,) δ |
| (1^{st} | | | | benzodioxine-6-sulfonyl)- | | (ppm): 7.37-7.25 (m, |
| eluting | | | | 1H,2H,3H,4H,5H,6H-pyrrolo[3,4- | | 7H), 7.00-6.98(m, 1H) |
| isomer) | | | | c]pyrrol-2-yl]-3-(3-fluoroazetidin- | | 4.37-4.33 (m, 1H), |
| 105-2 | +++ | 2-2 | | 3-yl)-2-phenylpropan-1-one; | | 4.30-4.26 (m, 4H), |
| (2^{nd} | | | | (2R)-1-[5-(2,3-dihydro-1,4- | | 4.15-3.96 (m, 6H), |
| eluting | | | | benzodioxine-6-sulfonyl)- | | 3.93-3.89 (m, 2H), |
| isomer) | | | | 1H,2H,3H,4H,5H,6H-pyrrolo[3,4- | | 3.77-3.65 (m, 2H), |
| | | | | c]pyrrol-2-yl]-3-(3-fluoroazetidin- | | 3.46-3.39 (m, 1H), |
| | | | | 3-yl)-2-phenylpropan-1-one | | 3.19-3.12 (m, 1H), |
| | | | | | | 2.72-2.62 (m, 1H), |
| | | | | | | 2.39-2.27 (m, 1H). |
| 106-1 | ++ | 2-2 | M | N-[(1S)-2-[5-(2,3-dihydro-1,4- | 520.3 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-oxo-1- | | |
| | | | | phenylethyl]methanesulfonamide; | | |
| | | | | or N-[(1R)-2-[5-(2,3-dihydro-1,4- | | |
| | | | | benzodioxine-6-sulfonyl)- | | |
| | | | | 1H,2H,3H,4H,5H,6H-pyrrolo[3,4- | | |
| | | | | c]pyrrol-2-yl]-2-oxo-1- | | |
| | | | | phenylethyl]methanesulfonamide | | |
| 106-2 | +++ | 2-2 | M | 1-[(2R)-3-[5-(2,3-dihydro-1,4- | 513.4 | (CD₃OD, 300 MHz) δ |
| | | | | benzodioxine-6-sulfonyl)- | | (ppm): 7.37-7.25 (m, |
| | | | | 1H,2H,3H,4H,5H,6H-pyrrolo[3,4- | | 7H), 7.02-6.99 (m, |
| | | | | c]pyrrol-2-yl]-3-oxo-2- | | 1H), 4.32-4.30 (m, |
| | | | | phenylpropyl]-3-methylurea; or 1- | | 5H), 4.20-3.95 (m, |
| | | | | [(2S)-3-[5-(2,3-dihydro-1,4- | | 7H), 3.84-3.79 (m, |
| | | | | benzodioxine-6-sulfonyl)- | | 1H), 3.61-3.41 (m, |
| | | | | 1H,2H,3H,4H,5H,6H-pyrrolo[3,4- | | 2H), 2.66 (s, 3H). |
| | | | | c]pyrrol-2-yl]-3-oxo-2- | | |
| | | | | phenylpropyl]-3-methylurea | | |
| 106-3 | ++ | 2-2 | M | 1-[5-(2,3-dihydro-1,4- | 455.1 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-methyl-2- | | |
| | | | | phenylpropan-1-one | | |
| 106-4 | ++ | 2-2 | M | 1-[5-(2,3-dihydro-1,4- | 485.0 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-phenyl-2-(propan- | | |
| | | | | 2-yloxy)ethan-1-one | | |
| 106-5 | ++ | 2-2 | M | (2S)-1-[5-(2,3-dihydro-1,4- | 457.0 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-methoxy-2- | | |
| | | | | phenylethan-1-one; or (2R)-1-[5- | | |
| | | | | (2,3-dihydro-1,4-benzodioxine-6- | | |
| | | | | sulfonyl)-1H,2H,3H,4H,5H,6H- | | |
| | | | | pyrrolo[3,4-c]pyrrol-2-yl]-2- | | |
| | | | | methoxy-2-phenylethan-1-one | | |
| 106-6 | ++ | 2-2 | M | 2-(2,3-dihydro-1,4-benzodioxine- | 453.1 | |
| | | | | 6-sulfonyl)-5-(1- | | |
| | | | | phenylcyclopropanecarbonyl)- | | |
| | | | | 1H,2H,3H,4H,5H,6H-pyrrolo[3,4- | | |
| | | | | c]pyrrole | | |
| 106-8 | ++ | 2-2 | M | 2-cyclopropyl-1-[5-(2,3-dihydro- | 467.1 | |
| | | | | 1,4-benzodioxine-6-sulfonyl)- | | |
| | | | | 1H,2H,3H,4H,5H,6H-pyrrolo[3,4- | | |
| | | | | c]pyrrol-2-yl]-2-phenylethan-1- | | |
| | | | | one | | |
| 107-1 | ++ | — | N | (2S)-1-[5-(3,4-dihydro-2H-1- | 539.5 | |
| | | | | benzopyran-6-sulfonyl)- | | |
| | | | | 1H,2H,3H,4H,5H,6H-pyrrolo[3,4- | | |
| | | | | c]pyrrol-2-yl]-2-hydroxy-2-[3-(4- | | |
| | | | | methylpiperazin-1- | | |
| | | | | yl)phenyl]ethan-1-one; or (2R)-1- | | |
| | | | | [5-(3,4-dihydro-2H-1-benzopyran- | | |
| | | | | 6-sulfonyl)-1H,2H,3H,4H,5H,6H- | | |
| | | | | pyrrolo[3,4-c]pyrrol-2-yl]-2- | | |
| | | | | hydroxy-2-[3-(4-methylpiperazin- | | |
| | | | | 1-yl)phenyl]ethan-1-one | | |
| 107-2 | ++ | — | N | (2S)-2-hydroxy-2-[3-(4- | 535.5 | |
| | | | | methylpiperazin-1-yl)phenyl]-1- | | |
| | | | | [5-(quinoxaline-6-sulfonyl)- | | |
| | | | | 1H,2H,3H,4H,5H,6H-pyrrolo[3,4- | | |
| | | | | c]pyrrol-2-yl]ethan-1-one; or (2R)- | | |
| | | | | 2-hydroxy-2-[3-(4- | | |
| | | | | methylpiperazin-1-yl)phenyl]-1- | | |
| | | | | [5-(quinoxaline-6-sulfonyl)- | | |
| | | | | 1H,2H,3H,4H,5H,6H-pyrrolo[3,4- | | |
| | | | | c]pyrrol-2-yl]ethan-1-one | | |
| 107-3 | ++ | — | N | (2S)-2-hydroxy-1-{5-[(4-methyl- | 554.5 | |
| | | | | 3,4-dihydro-2H-1,4-benzoxazin-6- | | |
| | | | | yl)sulfonyl]-1H,2H,3H,4H,5H,6H- | | |
| | | | | pyrrolo[3,4-c]pyrrol-2-yl}-2-[3-(4- | | |
| | | | | methylpiperazin-1- | | |
| | | | | yl)phenyl]ethan-1-one; or (2R)-2- | | |
| | | | | hydroxy-1-{5-[(4-methyl-3,4- | | |
| | | | | dihydro-2H-1,4-benzoxazin-6- | | |
| | | | | yl)sulfonyl]-1H,2H,3H,4H,5H,6H- | | |
| | | | | pyrrolo[3,4-c]pyrrol-2-yl}-2-[3-(4- | | |
| | | | | methylpiperazin-1- | | |
| | | | | yl)phenyl]ethan-1-one | | |
| 107-4 | ++ | — | N | (2S)-1-[5-(1,3-benzothiazole-5- | 529.4 | |
| | | | | sulfonyl)-1H,2H,3H,4H,5H,6H- | | |
| | | | | pyrrolo[3,4-c]pyrrol-2-yl]-2- | | |
| | | | | hydroxy-2-{3-[(2- | | |
| | | | | methoxyethyl)(methyl)amino]phenyl}ethan- | | |
| | | | | 1-one; or (2R)-1-[5- | | |
| | | | | (1,3-benzothiazole-5-sulfonyl)- | | |
| | | | | 1H,2H,3H,4H,5H,6H-pyrrolo[3,4- | | |
| | | | | c]pyrrol-2-yl]-2-hydroxy-2-{3-[(2- | | |
| | | | | methoxyethyl)(methyl)amino]phenyl}ethan- | | |
| | | | | 1-one | | |
| 108-1 | +++ | 43-1 | O | (2S)-1-[5-(1-benzofuran-6- | 480.4 | |
| | | | | sulfonyl)-1H,2H,3H,4H,5H,6H- | | |
| | | | | pyrrolo[3,4-c]pyrrol-2-yl]-2- | | |
| | | | | hydroxy-2-(2-methyl-1,3- | | |
| | | | | benzoxazol-4-yl)ethan-1-one; or | | |
| | | | | (2R)-1-[5-(1-benzofuran-6- | | |
| | | | | sulfonyl)-1H,2H,3H,4H,5H,6H- | | |
| | | | | pyrrolo[3,4-c]pyrrol-2-yl]-2- | | |
| | | | | hydroxy-2-(2-methyl-1,3- | | |
| | | | | benzoxazol-4-yl)ethan-1-one | | |
| 108-2 | + | 2-2 | O | (2S)-1-[5-(2,3-dihydro-1,4- | 443.2 | (DMSO-d₆, 300 MHz) |
| | | | | benzodioxine-6-sulfonyl)- | | δ (ppm): 7.35-7.23 (m, |
| | | | | 1H,2H,3H,4H,5H,6H-pyrrolo[3,4- | | 7H), 7.07-7.04 (m, |
| | | | | c]pyrrol-2-yl]-2-hydroxy-2- | | 1H), 5.65-6.63 (m, |
| | | | | phenylethan-1-one | | 1H), 5.18-5.16 (m, |
| | | | | | | 1H), 4.32-4.22 (m, |
| | | | | | | 5H), 4.07-3.93 (m, |
| | | | | | | 7H). |
| 108-3 | ++ | 2-2 | O | N-[(1S)-2-[5-(2,3-dihydro-1,4- | 484.1 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-oxo-1- | | |
| | | | | phenylethyl]acetamide; or N- | | |
| | | | | [(1R)-2-[5-(2,3-dihydro-1,4- | | |
| | | | | benzodioxine-6-sulfonyl)- | | |
| | | | | 1H,2H,3H,4H,5H,6H-pyrrolo[3,4- | | |
| | | | | c]pyrrol-2-yl]-2-oxo-1- | | |
| | | | | phenylethyl]acetamide | | |
| 109-1 | +++ | 2-2, 63-2 | OA | 1-[5-(2,3-dihydro-1,4- | 528.2 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-hydroxy-2-[3- | | |
| | | | | (morpholin-4-yl)phenyl]ethan-1- | | |
| | | | | one | | |
| 109-2 | ++++ | 63-9, 2-2, | OA | (2S)-2-{3-[(3aR,6aR)-3a-fluoro-5- | 585.0 | 1^{st}: (DMSO-d₆, 400 |
| (1^{st} | | 86 | | methyl-octahydropyrrolo[3,4- | | MHz) δ (ppm): 7.30- |
| eluting | | | | c]pyrrol-2-yl]phenyl}-1-[5-(2,3- | | 7.23 (m, 2H), 7.17-7.13 |
| isomer) | | | | dihydro-1,4-benzodioxine-6- | | (m, 1H), 7.06 (d, J = |
| | | | | sulfonyl)-1H,2H,3H,4H,5H,6H- | | 8.4 Hz, 1H), 6.72-6.66 |
| | | | | pyrrolo[3,4-c]pyrrol-2-yl]-2- | | (m, 2H), 6.63-6.61 (m, |
| | | | | hydroxyethan-1-one; | | 1H), 5.52 (d, J = 6.8 |
| | | | | (2R)-2-{3-[(3aS,6aS)-3a-fluoro-5- | | Hz, 1H), 5.07 (d, J = |
| | | | | methyl-octahydropyrrolo[3,4- | | 6.4 Hz, 1H), 4.33-4.29 |
| | | | | c]pyrrol-2-yl]phenyl}-1-[5-(2,3- | | (m, 4H), 4.26-4.22 (m, |
| | | | | dihydro-1,4-benzodioxine-6- | | 1H), 4.14-3.82 (m, |
| | | | | sulfonyl)-1H,2H,3H,4H,5H,6H- | | 7H), 3.65-3.60 (m, |
| | | | | pyrrolo[3,4-c]pyrrol-2-yl]-2- | | 1H), 3.58-3.40 (m, |
| | | | | hydroxyethan-1-one; | | 2H), 3.02-2.71 (m, |
| | | | | (2S)-2-{3-[(3aS,6aS)-3a-fluoro-5- | | 5H), 2.38-2.31 (m, |
| | | | | methyl-octahydropyrrolo[3,4- | | 1H), 2.25 (s, 3H). |
| 109-3 | ++++ | | | c]pyrrol-2-yl]phenyl}-1-[5-(2,3- | | 2^{nd}: (DMSO-d₆, 400 |
| (2^{nd} | | | | dihydro-1,4-benzodioxine-6- | | MHz) δ (ppm): 7.30- |
| eluting | | | | sulfonyl)-1H,2H,3H,4H,5H,6H- | | 7.23 (m, 2H), 7.17-7.13 |
| isomer) | | | | pyrrolo[3,4-c]pyrrol-2-yl]-2- | | (m, 1H), 7.06 (d, J = |
| | | | | hydroxyethan-1-one; | | 8.4 Hz, 1H), 6.72-6.66 |
| | | | | (2R)-2-{3-[(3aR,6aR)-3a-fluoro-5- | | (m, 2H), 6.63-6.61 (m, |
| | | | | methyl-octahydropyrrolo[3,4- | | 1H), 5.52 (d, J = 6.4 |
| | | | | c]pyrrol-2-yl]phenyl}-1-[5-(2,3- | | Hz, 1H), 5.06 (d, J = |
| | | | | dihydro-1,4-benzodioxine-6- | | 6.4 Hz, 1H), 4.33-4.20 |
| | | | | sulfonyl)-1H,2H,3H,4H,5H,6H- | | (m, 5H), 4.14-3.82 (m, |
| | | | | pyrrolo[3,4-c]pyrrol-2-yl]-2- | | 7H), 3.65-3.40 (m, |
| | | | | hydroxyethan-1-one | | 3H), 3.02-2.71 (m, |
| | | | | | | 5H), 2.38-2.31 (m, |
| | | | | | | 1H), 2.26 (s, 3H). |
| 109-4 | ++ | | | | | 3^{rd}: (DMSO-d₆, 400 |
| (3^{rd} | | | | | | MHz) δ (ppm): 7.30- |
| eluting | | | | | | 7.23 (m, 2H), 7.17-7.13 |
| isomer) | | | | | | (m, 1H), 7.06 (d, J = |
| | | | | | | 8.4 Hz, 1H), 6.72-6.66 |
| | | | | | | (m, 2H), 6.63-6.61 (m, |
| | | | | | | 1H), 5.52 (d, J = 6.4 |
| | | | | | | Hz, 1H), 5.07 (d, J = |
| | | | | | | 6.4 Hz, 1H), 4.33-4.29 |
| | | | | | | (m, 4H), 4.26-4.22 (m, |
| | | | | | | 1H), 4.14-3.82 (m, |
| | | | | | | 7H), 3.65-3.60 (m, |
| | | | | | | 1H), 3.58-3.40 (m, |
| | | | | | | 2H), 3.02-2.71 (m, |
| | | | | | | 5H), 2.38-2.31 (m, |
| | | | | | | 1H), 2.25 (s, 3H). |
| 109-5 | ++ | | | | | 4^{th}: (DMSO-d₆, 400 |
| (4^{th} | | | | | | MHz) δ (ppm): 7.30- |
| eluting | | | | | | 7.23 (m, 2H), 7.17-7.13 |
| isomer) | | | | | | (m, 1H), 7.06 (d, J = |
| | | | | | | 8.4 Hz, 1H), 6.72-6.66 |
| | | | | | | (m, 2H), 6.63-6.61 (m, |
| | | | | | | 1H), 5.52 (d, J = 6.4 |
| | | | | | | Hz, 1H), 5.07 (d, J = |
| | | | | | | 6.4 Hz, 1H), 4.33-4.29 |
| | | | | | | (m, 4H), 4.26-4.22 (m, |
| | | | | | | 1H), 4.14-3.82 (m, |
| | | | | | | 7H), 3.65-3.60 (m, |
| | | | | | | 1H), 3.58-3.40 (m, |
| | | | | | | 2H), 3.02-2.71 (m, |
| | | | | | | 5H), 2.38-2.31 (m, |
| | | | | | | 1H), 2.25 (s, 3H). |
| 109-6 | +++ | 63-2, 73 | OA | (2S)-2-{3-[(3S)-3- | 577.0 | 1^{st}: (DMSO-d₆, 300 |
| (1^{st} | | | | (difluoromethyl)piperazin-1- | | MHz) δ (ppm): 7.35- |
| eluting | | | | yl]phenyl}-1-[5-(2,3-dihydro-1,4- | | 7.30 (m, 2H), 7.28-7.26 |
| isomer) | | | | benzodioxine-6-sulfonyl)- | | (m, 1H), 6.99-6.85 (m, |
| | | | | 1H,2H,3H,4H,5H,6H-pyrrolo[3,4- | | 4H), 6.05-5.65 (m, |
| | | | | c]pyrrol-2-yl]-2-hydroxyethan-1- | | 1H), 4.96 (s, 1H), 4.34- |
| | | | | one; | | 4.30 (m, 5H), 4.19-4.07 |
| | | | | (2S)-2-{3-[(3R)-3- | | (m, 5H), 4.01-3.90 (m, |
| | | | | (difluoromethyl)piperazin-1- | | 1H), 3.66-3.64 (m, |
| | | | | yl]phenyl}-1-[5-(2,3-dihydro-1,4- | | 2H), 3.53-3.51 (m, |
| | | | | benzodioxine-6-sulfonyl)- | | 1H), 3.29-3.24 (m, |
| | | | | 1H,2H,3H,4H,5H,6H-pyrrolo[3,4- | | 2H), 3.10-2.84 (m, |
| | | | | c]pyrrol-2-yl]-2-hydroxyethan-1- | | 3H). |
| 109-7 | +++ | | | one; (2R)-2-{3-[(3S)-3- | | 2^{nd}: (DMSO-d₆, 300 |
| (2^{nd} | | | | (difluoromethyl)piperazin-1- | | MHz) δ (ppm): 7.35- |
| eluting | | | | yl]phenyl}-1-[5-(2,3-dihydro-1,4- | | 7.25 (m, 3H), 6.99-6.84 |
| isomer) | | | | benzodioxine-6-sulfonyl)- | | (m, 4H), 6.00-5.60 (m, |
| | | | | 1H,2H,3H,4H,5H,6H-pyrrolo[3,4- | | 1H), 4.96 (s, 1H), 4.34- |
| | | | | c]pyrrol-2-yl]-2-hydroxyethan-1- | | 4.30 (m, 5H), 4.19-4.01 |
| | | | | one; or | | (m, 6H), 3.67-3.58 (m, |
| | | | | 2R)-2-{3-[(3R)-3- | | 2H), 3.51-3.48 (m, |
| | | | | (difluoromethyl)piperazin-1- | | 1H), 3.25-3.19 (m, |
| | | | | yl]phenyl}-1-[5-(2,3-dihydro-1,4- | | 2H), 3.07-2.77 (m, |
| | | | | benzodioxine-6-sulfonyl)- | | 3H). |
| | | | | 1H,2H,3H,4H,5H,6H-pyrrolo[3,4- | | |
| | | | | c]pyrrol-2-yl]-2-hydroxyethan-1- | | |
| | | | | one; | | |
| 109-8 | +++ | 2-2, 78 | OA | (2S)-1-[5-(2,3-dihydro-1,4- | 539.5 | (DMSO-d₆, 400 MHz) |
| | | | | benzodioxine-6-sulfonyl)- | | δ (ppm): 7.28-7.23 (m, |
| | | | | 1H,2H,3H,4H,5H,6H-pyrrolo[3,4- | | 2H), 7.13-7.04 (m, |
| | | | | c]pyrrol-2-yl]-2-[3-(4- | | 2H), 6.80-6.76 (m, |
| | | | | methylpiperazin-1- | | 2H), 6.65-6.63 (m, |
| | | | | yl)phenyl]propan-1-one; or (2R)- | | 1H), 4.33-4.29 (m, |
| | | | | 1-[5-(2,3-dihydro-1,4- | | 5H), 4.04-3.90 (m, |
| | | | | benzodioxine-6-sulfonyl)- | | 6H), 3.83-3.69 (m, |
| | | | | 1H,2H,3H,4H,5H,6H-pyrrolo[3,4- | | 2H), 3.09-3.07 (m, |
| | | | | c]pyrrol-2-yl]-2-[3-(4- | | 4H), 2.44-2.41 (m, |
| | | | | methylpiperazin-1- | | 4H), 2.21 (s, 3H), 1.26- |
| | | | | yl)phenyl]propan-1-one | | 1.24 (m, 3H). |
| 109-9 | ++++ | 2-2 | OA | 1-[5-(2,3-dihydro-1,4- | 527.2 | |
| | | | | benzodioxine-6-sulfonyl)- | | |
| | | | | 1H,2H,3H,4H,5H,6H-pyrrolo[3,4- | | |
| | | | | c]pyrrol-2-yl]-2-hydroxy-2-[3- | | |
| | | | | (piperazin-1-yl)phenyl]ethan-1- | | |
| | | | | one | | |
| 109-10 | +++ | 2-2, 63-2 | OA | (2S)-2-[3-(4-cyclopropylpiperazin- | 567.5 | |
| | | | | 1-yl)pheny1]-1-[5-(2,3-dihydro- | | |
| | | | | 1,4-benzodioxine-6-sulfonyl)- | | |
| | | | | 1H,2H,3H,4H,5H,6H-pyrrolo[3,4- | | |
| | | | | c]pyrrol-2-yl]-2-hydroxyethan-1- | | |
| | | | | one; or (2R)-2-[3-(4- | | |
| | | | | cyclopropylpiperazin-1- | | |
| | | | | yl)phenyl]-1-[5-(2,3-dihydro-1,4- | | |
| | | | | benzodioxine-6-sulfonyl)- | | |
| | | | | 1H,2H,3H,4H,5H,6H-pyrrolo[3,4- | | |
| | | | | c]pyrrol-2-yl]-2-hydroxyethan-1- | | |
| | | | | one | | |
| 109-11 | ++++ | — | OA | (2S)-1-[5-(1-benzofuran-6- | 523.5 | |
| | | | | sulfonyl)-1H,2H,3H,4H,5H,6H- | | |
| | | | | pyrrolo[3,4-c]pyrrol-2-yl]-2- | | |
| | | | | hydroxy-2-[3-(4-methylpiperazin- | | |
| | | | | 1-yl)phenyl]ethan-1-one; or (2R)- | | |
| | | | | 1-[5-(1-benzofuran-6-sulfonyl)- | | |
| | | | | 1H,2H,3H,4H,5H,6H-pyrrolo[3,4- | | |
| | | | | c]pyrrol-2-yl]-2-hydroxy-2-[3-(4- | | |
| | | | | methylpiperazin-1- | | |
| | | | | yl)phenyl]ethan-1-one | | |
| 110-1 | ++ | 94-27, 94-28 | P | (2S)-1-(5-{2H,3H- | 513.2 | (CD₃OD, 300 MHz) δ |
| (1^{st} | | | | [1,4]dioxino[2,3-b]pyridine-7- | | (ppm): 8.18-8.16 (m, |
| eluting | | | | sulfonyl}-1H,2H,3H,4H,5H,6H- | | 1H), 7.67-7.65 (m, |
| isomer) | | | | pyrrolo[3,4-c]pyrrol-2-yl)-2- | | 1H), 7.57-7.55 (m, |
| 110-2 | −− | | | methoxy-2-(2-methyl-1,3- | | 1H), 7.44-7.35 (m, 2H), |
| (2^{nd} | | | | benzoxazol-4-yl)ethan-1-one; | | 5.60 (s, 1H), 4.55- |
| eluting | | | | (2R)-1-(5-{2H,3H- | | 4.52 (m, 3H), 4.48-3.98 |
| isomer) | | | | [1,4]dioxino[2,3-b]pyridine-7- | | (m, 9H), 3.41 (s, 3H), |
| | | | | sulfonyl}-1H,2H,3H,4H,5H,6H- | | 2.67 (s, 3H). |
| | | | | pyrrolo[3,4-c]pyrrol-2-yl)-2- | | |
| | | | | methoxy-2-(2-methyl-1,3- | | |
| | | | | benzoxazol-4-yl)ethan-1-one | | |
| 111-1 | ++ | 54-1, 39-1, | Q | (2S)-1-(5-{2H,3H- | 569.5 | (DMSO-d₆, 400 MHz) |
| (1^{st} | | 63-3 | | [1,4]dioxino[2,3-b]pyridine-7- | | δ (ppm): 8.15-8.14 (m, |
| eluting | | | | sulfonyl}-1H,2H,3H,4H,5H,6H- | | 1H), 7.61-7.60 (m, |
| isomer) | | | | pyrrolo[3,4-c]pyrrol-2-yl)-2- | | 1H), 7.15-7.09 (m, |
| 111-2 | −− | | | hydroxy-2-(3-{2-oxa-7- | | 1H), 6.93-6.91 (m, |
| (2^{nd} | | | | azaspiro[3.5]nonan-7- | | 1H), 6.85-6.77 (m, |
| eluting | | | | yl}phenyl)ethan-1-one; | | 1H), 6.73-6.71 (m, |
| isomer) | | | | (2R)-1-(5-{2H,3H- | | 1H), 5.52-5.50 (m, |
| | | | | [1,4]dioxino[2,3-b]pyridine-7- | | 1H), 5.07-5.05 (m, |
| | | | | sulfonyl}-1H,2H,3H,4H,5H,6H- | | 1H), 4.50-4.46 (m, |
| | | | | pyrrolo[3,4-c]pyrrol-2-yl)-2- | | 2H), 4.33-4.30 (m, |
| | | | | hydroxy-2-(3-{2-oxa-7- | | 6H), 4.24-4.22 (m, |
| | | | | azaspiro[3.5]nonan-7- | | 1H), 4.03 (s, 5H), 4.01- |
| | | | | yl}phenyl)ethan-1-one | | 3.81 (m, 2H), 3.05-3.02 |
| | | | | | | (m, 4H), 1.87-1.84 (m, |
| | | | | | | 4H). |
| 112-1 | +++ | 7-4, 39-1 | R | (2S)-2-(2,3-dihydro-1-benzofuran- | 486.3 | (DMSO-d₆, 400 MHz) |
| (1^{st} | | | | 7-yl)-1-(5-{2H,3H- | | δ (ppm): 8.15 (d, J = |
| eluting | | | | [1,4]dioxino[2,3-b]pyridine-7- | | 2.4 Hz, 1H), 7.62 (d, |
| isomer) | | | | sulfonyl}-1H,2H,3H,4H,5H,6H- | | J = 2.0 Hz, 1H), 7.14 (d, |
| 112-2 | −− | | | pyrrolo[3,4-c]pyrrol-2-yl)-2- | | J = 6.8 Hz, 1H), 7.05 |
| (2^{nd} | | | | hydroxyethan-1-one; | | (d, J = 7.2 Hz, 1H), |
| eluting | | | | (2R)-2-(2,3-dihydro-1-benzofuran- | | 6.77 (t, J = 7.6 Hz, 1H), |
| isomer) | | | | 7-yl)-1-(5-{2H,3H- | | 5.37 (d, J = 6.8 Hz, |
| | | | | [1,4]dioxino[2,3-b]pyridine-7- | | 1H), 5.22 (d, J = 6.8 |
| | | | | sulfonyl}-1H,2H,3H,4H,5H,6H- | | Hz, 1H), 4.56-4.46 (m, |
| | | | | pyrrolo[3,4-c]pyrrol-2-yl)-2- | | 4H), 4.34-4.24 (m, |
| | | | | hydroxyethan-1-one | | 3H), 4.10-4.06 (m, |
| | | | | | | 5H), 3.97-3.94 (m, |
| | | | | | | 1H), 3.79-3.76 (m, 1H), |
| | | | | | | 3.16 (t, J = 8.8 Hz, |
| | | | | | | 2H). |
| 112-3 | ++ | 7-5, 2-2, | R | (2S)-1-[5-(2,3-dihydro-1,4- | 548.0 | (DMSO-d₆, 400 MHz) |
| (1^{st} | | 5-4 | | benzodioxine-6-sulfonyl)- | | δ (ppm): 7.29-7.25 (m, |
| eluting | | | | 1H,2H,3H,4H,5H,6H-pyrrolo[3,4- | | 2H), 7.18-7.13 (m, |
| isomer) | | | | c]pyrrol-2-yl]-2-{3-[2- | | 2H), 7.06 (d, J = 8.4 |
| 112-4 | ++++ | | | (dimethylamino)ethoxy]-4- | | Hz, 1H), 6.91 (s, 1H), |
| (2^{nd} | | | | fluorophenyl}-2-hydroxyethan-1- | | 5.71 (d, J = 6.4 Hz, |
| eluting | | | | one; | | 1H), 5.14 (d, J = 6.4 |
| isomer) | | | | (2R)-1-[5-(2,3-dihydro-1,4- | | Hz, 1H), 4.32-4.25 (m, |
| | | | | benzodioxine-6-sulfonyl)- | | 5H), 4.10-3.96 (m, |
| | | | | 1H,2H,3H,4H,5H,6H-pyrrolo[3,4- | | 9H), 2.64-2.62 (m, |
| | | | | c]pyrrol-2-yl]-2-{3-[2- | | 2H), 2.21 (s, 6H). |
| | | | | (dimethylamino)ethoxy]-4- | | |
| | | | | fluorophenyl}-2-hydroxyethan-1- | | |
| | | | | one | | |
| 112-5 | ++++ | 2-2, 5-5, | R | (2S)-1-[5-(2,3-dihydro-1,4- | 548.0 | (DMSO-d₆, 400 MHz) |
| (1^{st} | | 7-6 | | benzodioxine-6-sulfonyl)- | | δ (ppm): 7.29-7.25 (m, |
| eluting | | | | 1H,2H,3H,4H,5H,6H-pyrrolo[3,4- | | 2H), 7.09-7.06 (m, |
| isomer) | | | | c]pyrrol-2-yl]-2-{5-[2- | | 2H), 6.90-6.87 (m, |
| 112-6 | ++ | | | (dimethylamino)ethoxy]-2- | | 2H), 5.87-5.83 (m, |
| (2^{nd} | | | | fluorophenyl}-2-hydroxyethan-1- | | 1H), 5.36-5.34 (m, |
| eluting | | | | one; | | 1H), 4.39-4.30 (m, |
| isomer) | | | | (2R)-1-[5-(2,3-dihydro-1,4- | | 5H), 4.10-3.98 (m, |
| | | | | benzodioxine-6-sulfonyl)- | | 8H), 3.88-3.84 (m, |
| | | | | 1H,2H,3H,4H,5H,6H-pyrrolo[3,4- | | 1H), 2.61-2.58 (m, |
| | | | | c]pyrrol-2-yl]-2-{5-[2- | | 2H), 2.19-2.17 (m, |
| | | | | (dimethylamino)ethoxy]-2- | | 6H). |
| | | | | fluorophenyl}-2-hydroxyethan-1- | | |
| | | | | one | | |
| 113-1 | +++ | 63-1, 2-2 | S | (2R)-3- | 538.5 | |
| | | | | [(cyclopentylmethyl)amino]-1-[5- | | |
| | | | | (2,3-dihydro-1,4-benzodioxine-6- | | |
| | | | | sulfonyl)-1H,2H,3H,4H,5H,6H- | | |
| | | | | pyrrolo[3,4-c]pyrrol-2-yl]-2- | | |
| | | | | phenylpropan-1-one; or (2S)-3- | | |
| | | | | [(cyclopentylmethyl)amino]-1-[5- | | |
| | | | | (2,3-dihydro-1,4-benzodioxine-6- | | |
| | | | | sulfonyl)-1H,2H,3H,4H,5H,6H- | | |
| | | | | pyrrolo[3,4-c]pyrrol-2-yl]-2- | | |
| | | | | phenylpropan-1-one | | |
| 114-1 | ++++ | 2-2, 83, | S | (2S)-2-(3-{9,9-difluoro-3,7- | 603.0 | (CD₃OD, 400 MHz) δ |
| (1^{st} | | 84, 66-3, | | diazabicyclo[3.3.1]nonan-3- | | (ppm): 7.33-7.26 (m, |
| eluting | | 85 | | yl}phenyl)-1-[5-(2,3-dihydro-1,4- | | 3H), 7.10-7.00 (m, |
| isomer) | | | | benzodioxine-6-sulfonyl)- | | 3H), 6.93-6.91 (m, |
| 114-2 | ++ | | | 1H,2H,3H,4H,5H,6H-pyrrolo[3,4- | | 1H), 5.20 (s, 1H), 4.33- |
| (2^{nd} | | | | c]pyrrol-2-yl]-2-hydroxyethan-1- | | 4.20 (m, 6H), 4.09-4.05 |
| eluting | | | | one; | | (m, 5H), 3.93-3.85 (m, |
| isomer) | | | | (2R)-2-(3-{9,9-difluoro-3,7- | | 3H), 3.36-3.33 (m, |
| | | | | diazabicyclo[3.3.1]nonan-3- | | 2H), 3.31-3.25 (m, |
| | | | | yl}phenyl)-1-[5-(2,3-dihydro-1,4- | | 2H), 3.17-3.12 (m, |
| | | | | benzodioxine-6-sulfonyl)- | | 2H), 2.22 (br s, 2H). |
| | | | | 1H,2H,3H,4H,5H,6H-pyrrolo[3,4- | | |
| | | | | c]pyrrol-2-yl]-2-hydroxyethan-1- | | |
| | | | | one | | |
| 114-3 | +++ | 63-1, 2-2 | S | (2R)-3- | 524.5 | |
| | | | | [(cyclobutylmethyl)amino]-1-[5- | | |
| | | | | (2,3-dihydro-1,4-benzodioxine-6- | | |
| | | | | sulfonyl)-1H,2H,3H,4H,5H,6H- | | |
| | | | | pyrrolo[3,4-c]pyrrol-2-yl]-2- | | |
| | | | | phenylpropan-1-one; or (2S)-3- | | |
| | | | | [(cyclobutylmethyl)amino]-1-[5- | | |
| | | | | (2,3-dihydro-1,4-benzodioxine-6- | | |
| | | | | sulfonyl)-1H,2H,3H,4H,5H,6H- | | |
| | | | | pyrrolo[3,4-c]pyrrol-2-yl]-2- | | |
| | | | | phenylpropan-1-one | | |
| 114-4 | +++ | 63-1, 2-2 | S | (2R)-1-[5-(2,3-dihydro-1,4- | 554.5 | |
| | | | | benzodioxine-6-sulfonyl)- | | |
| | | | | 1H,2H,3H,4H,5H,6H-pyrrolo[3,4- | | |
| | | | | c]pyrrol-2-yl]-3-[(oxan-4- | | |
| | | | | ylmethyl)amino]-2-phenylpropan- | | |
| | | | | 1-one; or (2S)-1-[5-(2,3-dihydro- | | |
| | | | | 1,4-benzodioxine-6-sulfonyl)- | | |
| | | | | 1H,2H,3H,4H,5H,6H-pyrrolo[3,4- | | |
| | | | | c]pyrrol-2-yl]-3-[(oxan-4- | | |
| | | | | ylmethyl)amino]-2-phenylpropan- | | |
| | | | | 1-one | | |
| 114-5 | +++ | 63-1, 2-2 | S | (2R)-1-[5-(2,3-dihydro-1,4- | 484.4 | |
| | | | | benzodioxine-6-sulfonyl)- | | |
| | | | | 1H,2H,3H,4H,5H,6H-pyrrolo[3,4- | | |
| | | | | c]pyrrol-2-yl]-3-(ethylamino)-2- | | |
| | | | | phenylpropan-1-one; or (2S)-1-[5- | | |
| | | | | (2,3-dihydro-1,4-benzodioxine-6- | | |
| | | | | sulfonyl)-1H,2H,3H,4H,5H,6H- | | |
| | | | | pyrrolo[3,4-c]pyrrol-2-yl]-3- | | |
| | | | | (ethylamino)-2-phenylpropan-1- | | |
| | | | | one | | |
| 115 | ++ | 41, 93 | T | (2S)-1-{5-[(2,3-dihydro-1,4- | 442.3 | |
| | | | | benzodioxin-6-yl)imino-λ⁶- | | |
| | | | | sulfanyl]-1H,2H,3H,4H,5H,6H- | | |
| | | | | pyrrolo[3,4-c]pyrrol-2-yl}-2- | | |
| | | | | hydroxy-2-phenylethan-1-one | | |

TABLE 23
Biological Data of Additional Compounds.

| Ex. | Compound | IC₅₀ (μM) |
|---|---|---|
| C-1 | 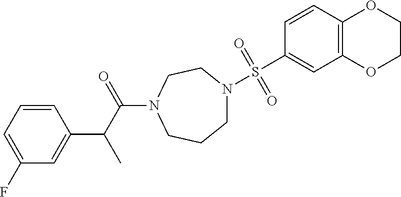 [SMILES: CC(C(=O)N1CCCN(S(=O)(=O)c2ccc3c(c2)OCCO3)CC1)c1cccc(F)c1] | (+) |
| C-2 | 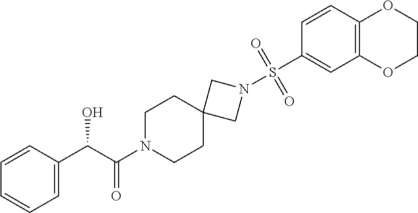 [SMILES: O=C([C@@H](O)c1ccccc1)N1CCC2(CC1)CN(S(=O)(=O)c1ccc3c(c1)OCCO3)C2] | (+) |
| C-3 | 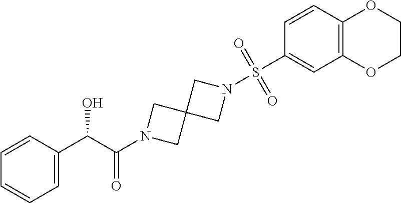 [SMILES: O=C([C@@H](O)c1ccccc1)N1CC2(C1)CN(S(=O)(=O)c1ccc3c(c1)OCCO3)C2] | (+) |
| C-4 | 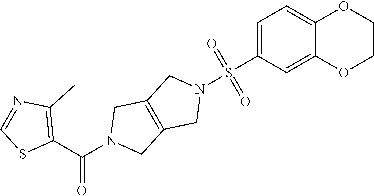 [SMILES: Cc1ncsc1C(=O)N1CC2=C(C1)CN(S(=O)(=O)c1ccc3c(c1)OCCO3)C2] | (+) |
| C-5 | 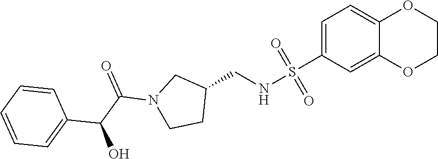 [SMILES: O=C([C@@H](O)c1ccccc1)N1CC[C@@H](CNS(=O)(=O)c2ccc3c(c2)OCCO3)C1] | (+) |
| C-6 | 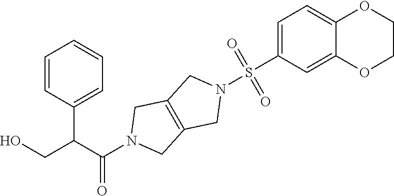 [SMILES: O=C(C(CO)c1ccccc1)N1CC2=C(C1)CN(S(=O)(=O)c1ccc3c(c1)OCCO3)C2] | (++) |
| C-7 | 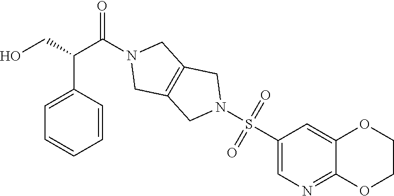 [SMILES: O=C([C@@H](CO)c1ccccc1)N1CC2=C(C1)CN(S(=O)(=O)c1cnc3c(c1)OCCO3)C2] | (++) |
| C-8 | 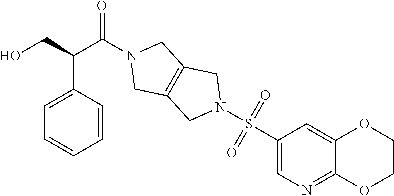 [SMILES: O=C([C@H](CO)c1ccccc1)N1CC2=C(C1)CN(S(=O)(=O)c1cnc3c(c1)OCCO3)C2] | (+) |
| C-9 | 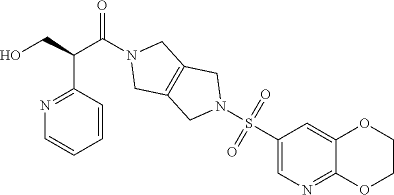 [SMILES: O=C([C@H](CO)c1ccccn1)N1CC2=C(C1)CN(S(=O)(=O)c1cnc3c(c1)OCCO3)C2] | (+) |

### Equivalents

Those skilled in the art will recognize, or be able to ascertain, using no more than routine experimentation, numerous equivalents to the specific embodiments described specifically herein. Such equivalents are intended to be encompassed in the scope of the following claims.

Embodiment 1. A compound of Formula I

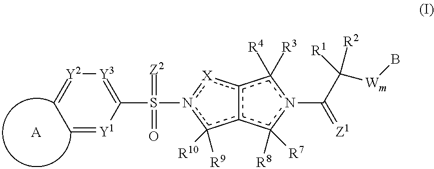

or a pharmaceutically acceptable salt thereof, wherein:
X is CR⁵R⁶, CR⁵, NR⁵, or N, as valency permits;
dashed bonds are each independently a single or a double bond, as valency permits,
   wherein at least one dashed bond is a double bond;
Y¹, Y², and Y³ are each independently N or CR^{a};
each R^{a} is independently —H, halogen, or —CN;
Ring A is a 5- to 6-membered aryl, 5- to 6-membered heteroaryl containing 1-4 heteroatoms independently selected from the group consisting of O, N, and S, 5- to 7-membered heterocyclyl containing 1-4 heteroatoms independently selected from the group consisting of O, N, and S, or 5- to 7-membered cycloalkyl,
   wherein each aryl, heteroaryl, heterocyclyl, or cycloalkyl is optionally substituted with one or more halogen, —C₁-C₆alkyl, —C₂-C₆alkenyl, —C₂-C₆alkynyl, oxo, or —C(O)R′;
Z¹ is O, S, or NR;
Z² is O or NR;
W is CR^{1′}R^{2′}, O, S, or NR;
m is 0 or 1;
R¹ and R² are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₁-C₁₄aryl, —(CR^{b}R^{c})ₙheteroaryl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each cycloalkyl, cycloalkenyl, heterocyclyl, aryl, or heteroaryl is optionally substituted with one or more R^{e},
   wherein each alkyl, alkenyl, or alkynyl is optionally substituted with one or more halogen,
   wherein each heterocyclyl is 3- to 14-membered and contains 1-4 heteroatoms independently selected from the group consisting of O, N, and S, and wherein the heterocyclyl does not contain an O in the γ-position relative to C(═Z¹), and
   wherein each heteroaryl is 5- to 14-membered and contains 1-4 heteroatoms independently selected from the group consisting of O, N, and S;
or R¹ and R² combine with the carbon to which they are attached to form oxo, a C₃-C₈cycloalkyl, or a 3- to 8-membered heterocyclyl containing 1-4 heteroatoms independently selected from the group consisting of O, N, and S, and wherein the heterocyclyl does not contain an O in the γ-position relative to C(═Z¹), and
   wherein each cycloalkyl or heterocyclyl is optionally substituted with one or more R^{e};
R^{1′} and R^{2′} are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each alkyl, alkenyl, alkynyl, cycloalkyl, cycloalkenyl, heterocyclyl, aryl, or heteroaryl is optionally substituted with one or more R^{e},
   wherein each heterocyclyl is 3- to 14-membered and contains 1-4 heteroatoms independently selected from the group consisting of O, N, and S, and
   wherein each heteroaryl is 5- to 14-membered and contains 1-4 heteroatoms independently selected from the group consisting of O, N, and S;
or R^{1′} and R^{2′} combine with the carbon to which they are attached to form oxo, a C₃-C₈cycloalkyl, or a 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e};
or R¹ and R^{1′} combine with the carbons to which they are attached to form a C₃-C₈cycloalkyl or 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e};
R^{b} and R^{c} are each independently selected from the group consisting of —H, halogen, and —C₁-C₆alkyl;
each n is independently 0, 1, 2, 3, or 4;
each R^{e} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S, and wherein —OR of R^{e} does not result in an O in the γ-position relative to C(═Z¹),
   wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
B is a monocyclic or bicyclic 3- to 14-membered ring,
   wherein the ring is saturated, fully or partially unsaturated, or aromatic, and
   wherein the ring contains 0-4 heteroatoms independently selected from the group consisting of O, N, and S,
   wherein the ring is optionally substituted with one or more R^{d}, and
   when m is 0 and the ring is saturated or partially unsaturated, then the ring does not contain an O in the γ-position relative to C(═Z¹);
each R^{d} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
each R³, R⁴, R⁵, R⁶, R⁷, R⁸, R⁹, and R¹⁰ is independently —H, —C₁-C₆alkyl, —C₃-C₈cycloalkyl, or 3- to 8-membered heterocyclyl containing 1-4 heteroatoms independently selected from the group consisting of O, N, and S,
   wherein each alkyl, cycloalkyl, or heterocyclyl is optionally substituted with one or more halogen, oxo, —C₁-C₆alkyl, —C₂-C₆alkenyl, —C₂-C₆alkynyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂,—C₃-C₈cycloalkyl, or 3- to 8-membered heterocyclyl containing 1-4 heteroatoms independently selected from the group consisting of O, N, and S, and
   wherein R³, R⁷, and R⁹ are each independently present or absent, as valency permits;
or R³ and R⁴, R⁵ and R⁶, R⁷ and R⁸, R⁹ and R¹⁰, or combinations thereof, combine with the carbon to which they are attached to form an oxo, C₃-C₈cycloalkyl, or 3- to 8-membered heterocyclyl containing 1-4 heteroatoms independently selected from the group consisting of O, N, and S;
each R is independently selected from the group consisting of —H, —OH, —O(C₁-C₆alkyl), —NH₂, —NH(C₁-C₆alkyl), —N(C₁-C₆alkyl)₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —O—C₁-C₆alkyl, —NH(C₁-C₆alkyl), —N(C₁-C₆alkyl)₂, —C₁-C₆alkyl optionally substituted with one or more oxo or —OH, —C₂-C₆alkenyl, —C₂-C₆alkynyl, —C₃-C₁₂cycloalkyl, or 3- to 8-membered heterocyclyl containing 1-4 heteroatoms independently selected from the group consisting of O, N, and S; and
each R′ is independently selected from the group consisting of —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —C₁-C₆alkyl optionally substituted with one or more oxo or —OH, —C₂-C₆alkenyl, —C₂-C₆alkynyl, —O—C₁-C₆alkyl, —NH(C₁-C₆alkyl), or —N(C₁-C₆alkyl)₂.

Embodiment 2. The compound of embodiment 1, wherein W is CR^{1′}R^{2′}.

Embodiment 3. The compound of embodiment 1 or 2, wherein R^{1′} and R^{2′} are each independently selected from the group consisting of —H, halogen, —C₁-C₆alkyl, —(CR^{b}R^{c})ₙC₃-C₁₂cycloalkyl, —(CR^{b}R^{c})ₙheterocyclyl, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, or —(CR^{b}R^{c})ₙNRC(O)OR,
wherein each alkyl, cycloalkyl, or heterocyclyl is optionally substituted with one or more substituents selected from the group consisting of halogen, —C₁-C₆alkyl, —OR, and oxo, and
wherein each heterocyclyl is 3- to 14-membered and contains 1-4 heteroatoms independently selected from the group consisting of O, N, and S and wherein the heterocyclyl does not contain an O in the γ-position relative to C(═Z¹);
or R^{1′} and R^{2′} combine with the carbon to which they are attached to form a C₃-C₈cycloalkyl or 3- to 8-membered heterocyclyl containing 1-4 heteroatoms independently selected from the group consisting of O, N and S,
   wherein the heterocyclyl does not contain an O in the γ-position relative to C(═Z¹);
or R and R^{1′} combine with the carbons to which they are attached to form a C₃-C₈cycloalkyl or 3- to 8-membered heterocyclyl containing 1-4 heteroatoms independently selected from the group consisting of O, N, and S,
   wherein each cycloalkyl or heterocyclyl is optionally substituted with one or more substituents independently selected from the group consisting of halogen, —C₁-C₆alkyl, —OR, and oxo, and
   wherein the heterocyclyl does not contain an O in the γ-position relative to C(═Z¹).

Embodiment 4. The compound of any one of embodiments 1-3, wherein R^{1′} and R^{2′} are each independently selected from the group consisting of —H, halogen, —C₁-C₆alkyl, —(CR^{b}R^{c})ₙC₃-C₁₂cycloalkyl, —(CR^{b}R^{c})ₙheterocyclyl, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, or —(CR^{b}R^{c})ₙNRC(O)OR,
wherein each alkyl, cycloalkyl, or heterocyclyl is optionally substituted with one or more substituents selected from the group consisting of halogen, —C₁-C₆alkyl, —OR, and oxo, and
wherein each heterocyclyl is 3- to 14-membered and contains 1-4 heteroatoms independently selected from the group consisting of O, N, and S and wherein the heterocyclyl does not contain an O in the γ-position relative to C(═Z¹);
or R^{1′} and R^{2′} combine with the carbon to which they are attached to form a C₃-C₈cycloalkyl or 3- to 8-membered heterocyclyl containing 1-4 heteroatoms independently selected from the group consisting of O, N and S,
   wherein the heterocyclyl does not contain an O in the γ-position relative to C(═Z¹).

Embodiment 5. The compound of any one of embodiments 1-4, wherein R^{1′} and R^{2′} are each —H.

Embodiment 6. The compound of embodiment 1, wherein m is 0.

Embodiment 7. The compound of any one of embodiments 1-6, wherein X is CR⁵R⁶, CR⁵, or N.

Embodiment 8. The compound of any one of embodiments 1-7, wherein Z¹ is O or S.

Embodiment 9. The compound of any one of embodiments 1-8, wherein Z² is O or NH.

Embodiment 10. The compound of any one of embodiments 1-9, wherein R³, R⁴, R⁵, R⁶, R⁷, R⁸, R⁹, and R¹⁰, if present, are each —H.

Embodiment 11. The compound of any one of embodiments 1-10, wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl is optionally substituted with one or more halogen or —C₁-C₆alkyl.

Embodiment 12. The compound of any one of embodiments 1-11, wherein the compound is of Formula II:

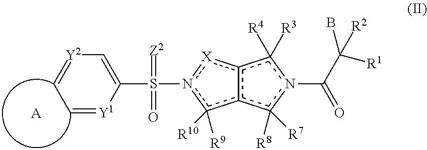

or a pharmaceutically acceptable salt thereof, wherein:
Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S,
   wherein each heteroaryl or heterocyclyl contains at least one oxygen atom and is optionally substituted with one or more halogen or —C₁-C₆alkyl.

Embodiment 13. The compound of any one of embodiments 1-12, wherein the compound is of Formula II-a:

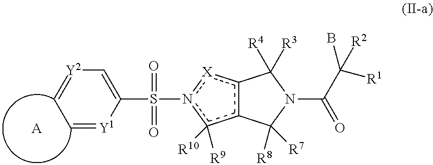

or a pharmaceutically acceptable salt thereof.

Embodiment 14. The compound of any one of embodiments 1-13, wherein the compound is of Formula II-b:

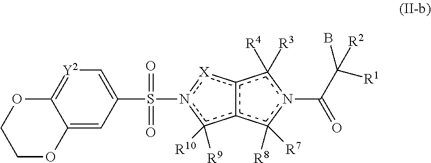

or a pharmaceutically acceptable salt thereof, wherein:

### Y² is CH or N.

Embodiment 15. The compound of any one of embodiments 1-14, wherein the compound is of Formula II-c:

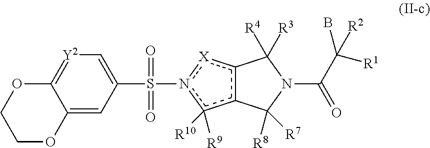

or a pharmaceutically acceptable salt thereof, wherein:

### Y² is CH or N.

Embodiment 16. The compound of any one of embodiments 1-15, wherein the compound is of Formula III:

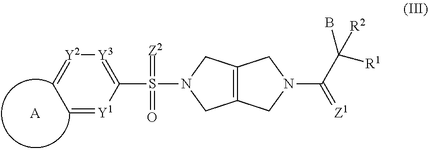

or a pharmaceutically acceptable salt thereof.

Embodiment 17. The compound of embodiment 16, wherein:
Y¹, Y², and Y³ are each independently N or CR^{a};
R^{a} is —H or halogen;
Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S,
   wherein each heteroaryl or heterocyclyl is optionally substituted with one or more halogen or —C₁-C₆alkyl;
Z¹ is O or S;
Z² is O or NR;
R¹ and R² are each independently —H, halogen, —C₁-C₆alkyl, —(CR^{b}R^{c})ₙC₃-C₁₂cycloalkyl, —(CR^{b}R^{c})ₙheterocyclyl, —OR, —(CR^{b}R^{c})ₙNR₂, —(CR^{b}R^{c})ₙNRC(O)R′, —(CR^{b}R^{c})ₙNRS(O)₂R′, or —(CR^{b}R^{c})ₙNRC(O)NR₂,
   wherein each heterocyclyl is optionally substituted with one or more substituents selected from the group consisting of halogen, —OR, and oxo, and wherein —OR does not result in an O in the γ-position relative to C(═Z¹), and
   wherein each heterocyclyl is 3- to 14-membered and contains 1-4 heteroatoms independently selected from the group consisting of O, N, and S, and wherein the heterocyclyl does not contain an O in the γ-position relative to C(═Z¹);
or R¹ and R² combine with the carbon to which they are attached to form a C₃-C₈cycloalkyl or 3- to 8-membered heterocyclyl containing 1-4 heteroatoms independently selected from the group consisting of O, N and S,
   wherein the heterocyclyl does not contain an O in the γ-position relative to C(═Z¹);
R^{b} and R^{c} are each independently —H;
n is 0, 1, or 2;
B is a monocyclic or bicyclic 3- to 14-membered ring,
   wherein the ring is saturated, fully or partially unsaturated, or aromatic, and
   wherein the ring contains 0-4 heteroatoms independently selected from the group consisting of O, N, and S,
   wherein the ring is optionally substituted with one or more R^{d}, and
   when the ring is saturated or partially unsaturated, then the ring does not contain an O in the γ-position relative to C(═Z);
each R^{d} is independently selected from the group consisting of halogen, —OR, —NR₂, and —C(O)NR₂, —C₁-C₆alkyl, —C₃-C₁₂cycloalkyl, 3- to 14-membered heterocyclyl containing 1-4 heteroatoms independently selected from the group consisting of O, N, and S, C₆-C₁₄aryl,
   wherein each alkyl, heterocyclyl, or aryl is optionally substituted with one or more substituents selected from the group consisting of halogen, —C₁-C₆alkyl optionally substituted with one or more halogen or —C₃-C₁₂cycloalkyl;
each R is independently selected from the group consisting of —H, —C₁-C₆alkyl, —C₃-C₁₂cycloalkyl, and 3- to 14-membered heterocyclyl containing 1-4 heteroatoms independently selected from the group consisting of O, N, and S,
   wherein each alkyl or heterocyclyl is optionally substituted with one or more halogen, —O—C₁-C₆alkyl,—NH—C₁-C₆alkyl, or —N(C₁-C₆alkyl)₂-C₁-C₆alkyl optionally substituted with —OH, —C₃-C₁₂cycloalkyl, or 3- to 8-membered heterocyclyl containing 1-4 heteroatoms independently selected from the group consisting of O, N, and S; and
each R′ is —C₁-C₆alkyl.

Embodiment 18. The compound of any one of embodiments 1-17, wherein the compound is of Formula III-a:

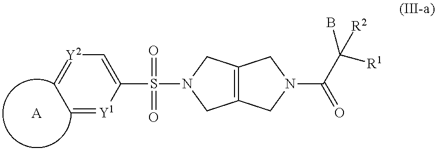

or a pharmaceutically acceptable salt thereof,
wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S,
   wherein each heteroaryl or heterocyclyl contains at least one oxygen atom and is optionally substituted with one or more halogen or —C₁-C₆alkyl.

Embodiment 19. The compound of any one of embodiments 1-18, wherein the compound is of Formula III-b:

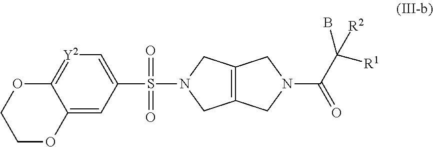

or a pharmaceutically acceptable salt thereof,
wherein Y² is CH or N.

Embodiment 20. The compound of any one of embodiments 1-19, wherein the compound is of Formula III-c:

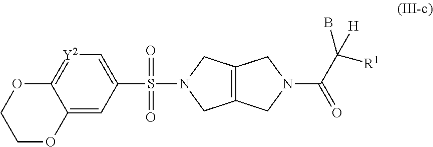

or a pharmaceutically acceptable salt thereof, wherein:
Y² is CH or N;
R¹ is —OH or —(CH₂)NHMe;
B is a phenyl ring,
   wherein the phenyl ring is optionally fused to an aromatic, saturated, or partially unsaturated 5- to 6-membered ring containing 0-4 heteroatoms independently selected from the group consisting of O, N, and S, and
   wherein each ring is optionally substituted with one or more R^{d};
each R^{d} is independently selected from the group consisting of halogen, —C₁-C₆alkyl, and —OR;
and
each R is independently —H, —C₁-C₆alkyl, or 3- to 8-membered heterocyclyl optionally substituted with —C₁-C₆alkyl.

Embodiment 21. The compound of any one of embodiments 1-11, wherein the compound is of Formula IV:

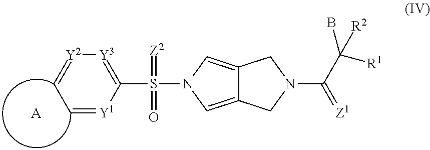

or a pharmaceutically acceptable salt thereof.

Embodiment 22. The compound of embodiment 21, wherein:
Y¹, Y², and Y³ are each independently N or CR^{a};
R^{a} is —H;
Ring A is a 6-membered heterocyclyl containing 0-4 heteroatoms independently selected from the group consisting of O, N, and S;
Z¹ is O;
Z² is O;
R¹ and R² are each independently —H, —OR, or —(CR^{b}R^{c})ₙNR₂;
R^{b} and R are each independently —H;
n is 1;
B is a monocyclic or bicyclic 3- to 14-membered ring,
   wherein the ring is partially unsaturated or aromatic, and
   wherein the ring contains 0-4 heteroatoms independently selected from the group consisting of O, N, and S, and
   wherein the ring is optionally substituted with one or more R^{d};
each R^{d} is independently selected from the group consisting of halogen, —C₁-C₆alkyl, and —OR; and
each R is independently selected from the group consisting of —H and —C₁-C₆alkyl.

Embodiment 23. The compound of any one of embodiments 1-11 and 21-22, wherein the compound is of Formula IV-a:

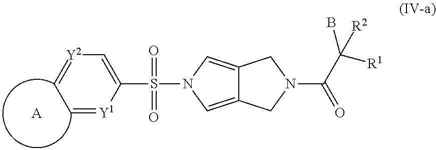

or a pharmaceutically acceptable salt thereof,
wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆alkyl.

Embodiment 24. The compound of any one of embodiments 1-11 and 21-23, wherein the compound is of Formula IV-b:

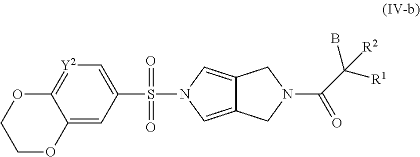

or a pharmaceutically acceptable salt thereof,
wherein Y² is CH or N.

Embodiment 25. The compound of any one of embodiments 1-11, wherein the compound is of Formula V:

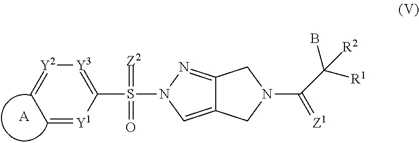

or a pharmaceutically acceptable salt thereof.

Embodiment 26. The compound of embodiment 25, wherein:
Y¹, Y², and Y³ are each independently CR^{a};
each R^{a} is —H;
Ring A is a 6-membered heterocyclyl containing 0-4 heteroatoms independently selected from the group consisting of O, N, and S;
Z¹ is O;
Z² is O;
R¹ and R² are each independently —H or —OR;
B is a monocyclic or bicyclic 3- to 14-membered ring,
   wherein the ring is partially unsaturated or aromatic, and
   wherein the ring contains 0-4 heteroatoms independently selected from the group consisting of O and N, and
   wherein the ring is optionally substituted with one or more —OR;
and
each R is independently selected from the group consisting of —H and —C₁-C₆alkyl.

Embodiment 27. The compound of any one of embodiments 1-11 and 25-26, wherein the compound is of Formula V-a:

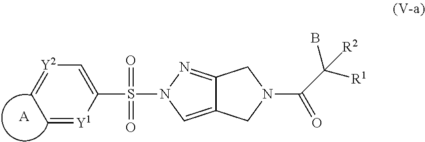

or a pharmaceutically acceptable salt thereof,
wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl contains at least one oxygen atom and is optionally substituted with one or more halogen or —C₁-C₆alkyl.

Embodiment 28. The compound of any one of embodiments 1-11 and 25-27, wherein the compound is of Formula V-b:

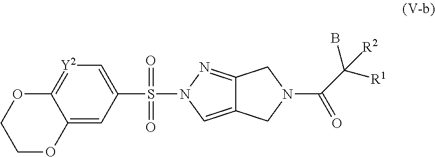

or a pharmaceutically acceptable salt thereof,
wherein Y² is CH or N.

Embodiment 29. The compound of any one of embodiments 1-28, wherein Y³ is CR^{a}.

Embodiment 30. The compound of any one of embodiments 1-29, wherein Y² is CR^{a}.

Embodiment 31. The compound of any one of embodiments 1-30, wherein R^{a} is —H.

Embodiment 32. The compound of any one of embodiments 1-31, wherein

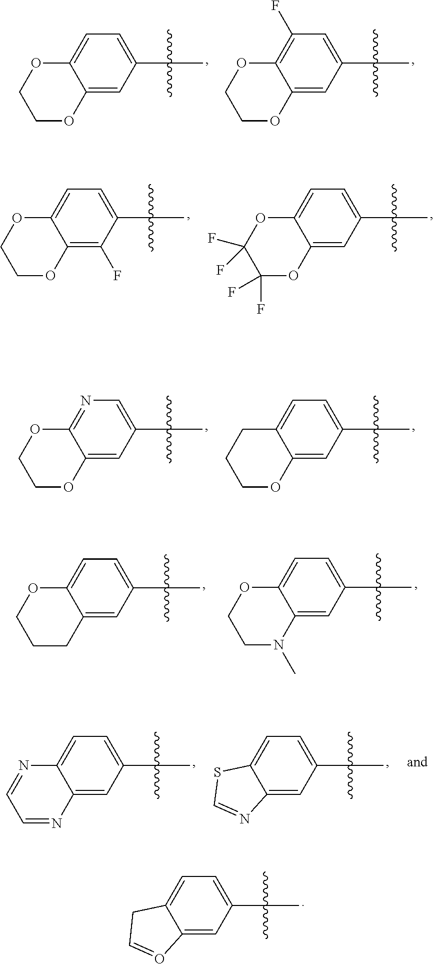

is selected from the group consisting of:

Embodiment 33. The compound of any one of embodiments 1-32, wherein

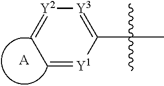

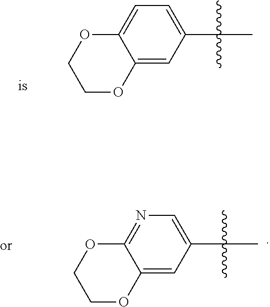 [SMILES: *c1ccc2c(c1)OCCO2 ; *c1cnc2c(c1)OCCO2]

Embodiment 34. The compound of any one of embodiments 1-33, wherein R¹ and R² are each independently —H, halogen, —C₁-C₆alkyl, —(CR^{b}R^{c})ₙC₃-C₁₂cycloalkyl, —(CR^{b}R^{c})ₙheterocyclyl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, or —(CR^{b}R^{c})ₙNRC(O)OR,
wherein each heterocyclyl is optionally substituted with one or more substituents selected from the group consisting of halogen, —OR, and oxo, and wherein —OR does not result in an O in the γ-position relative to C(═Z¹),
wherein each heterocyclyl is 3- to 14-membered and contains 1-4 heteroatoms independently selected from the group consisting of O, N, and S, and wherein the heterocyclyl does not contain an O in the γ-position relative to C(═Z¹);
or R¹ and R² combine with the carbon to which they are attached to form a C₃-C₈cycloalkyl or a 3- to 8-membered heterocyclyl containing 1-4 heteroatoms independently selected from the group consisting of O, N, and S,
   wherein the heterocyclyl does not contain an O in the γ-position relative to C(═Z¹).

Embodiment 35. The compound of any one of embodiments 1-34, wherein R¹ and R² are each independently selected from the group consisting of —H, halogen, —C₁-C₆alkyl, —(CR^{b}R^{c})ₙC₃-C₁₂cycloalkyl, —(CR^{b}R^{c})ₙheterocyclyl, —OR, —(CR^{b}R^{c})ₙNR₂, —(CR^{b}R^{c})ₙNRC(O)R′, —(CR^{b}R^{c})ₙNRS(O)₂R′, or —(CR^{b}R^{c})ₙNRC(O)NR₂,
wherein each heterocyclyl is optionally substituted with one or more substituents selected from the group consisting of halogen, —OR, and oxo, and wherein —OR does not result in an O in the γ-position relative to C(═Z¹), and
wherein each heterocyclyl is 3- to 14-membered and contains 1-4 heteroatoms independently selected from the group consisting of O, N, and S and wherein the heterocyclyl does not contain an O in the γ-position relative to C(═Z¹); or
or R¹ and R² combine with the carbon to which they are attached to form a C₃-C₈cycloalkyl or 3- to 8-membered heterocyclyl containing 1-4 heteroatoms independently selected from the group consisting of O, N, and S,
   wherein the heterocyclyl does not contain an O in the γ-position relative to C(═Z¹).

Embodiment 36. The compound of any one of embodiments 1-35, wherein R¹ and R² are each independently —H, —OH, or —CH₂NHMe.

Embodiment 37. The compound of any one of embodiments 1-36, wherein R² is —H.

Embodiment 38. The compound of any one of embodiments 1-37, wherein R¹ is —OH.

Embodiment 39. The compound of any one of embodiments 1-38, wherein R and R are each —H.

Embodiment 40. The compound of any one of embodiments 1-39, wherein n is 0, 1, or 2.

Embodiment 41. The compound of any one of embodiments 1-40, wherein B is a phenyl ring or a bicyclic ring,
wherein at least one of the rings in the bicyclic ring is a phenyl ring,
wherein the phenyl ring or bicyclic ring contains 0-4 heteroatoms independently selected from the group consisting of O, N, and S, and
wherein the phenyl ring or bicyclic ring is optionally substituted with one or more R^{d}.

Embodiment 42. The compound of any one of embodiments 1-41, wherein B is a ring selected from the group consisting of:

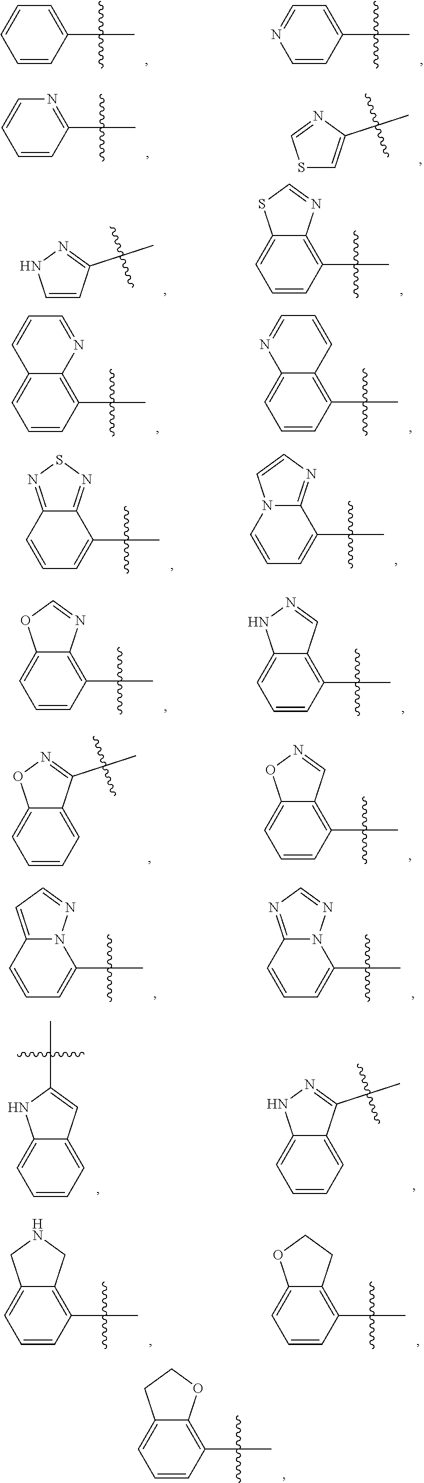

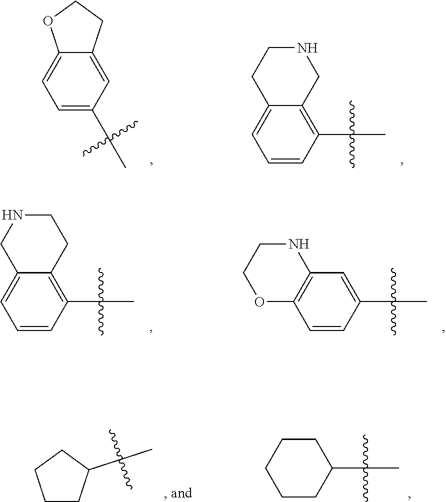 [SMILES: *c1ccc2c(c1)CCO2 ; *c1cccc2c1CNCC2 ; *c1cccc2c1CCNC2 ; *c1ccc2c(c1)NCCO2 ; *C1CCCC1 ; *C1CCCCC1]

and wherein the ring is optionally substituted with one or more R^{d}.

Embodiment 43. The compound of any one of embodiments 1-42, wherein B is phenyl, optionally substituted with one or more R^{d}.

Embodiment 44. The compound of any one of embodiments 1-43, wherein each R^{d} is independently selected from the group consisting of halogen, —OR, —NR₂, and —C(O)NR₂, —C₁-C₆alkyl, —C₃-C₁₂cycloalkyl, 3- to 14-membered heterocyclyl containing 1-4 heteroatoms independently selected from the group consisting of O, N, and S, and C₆-C₁₄aryl,
wherein each alkyl, heterocyclyl, or aryl is optionally substituted with one or more substituents selected from the group consisting of halogen, —C₁-C₆alkyl optionally substituted with one or more halogen, or —C₃-C₁₂cycloalkyl.

Embodiment 45. The compound of any one of embodiments 1-44, wherein each R^{d} is independently selected from the group consisting of halogen, —C₁-C₆alkyl, and —OR.

Embodiment 46. The compound of any one of embodiments 1-45, wherein each R is independently selected from the group consisting of —H, —C₁-C₆alkyl, —C₃-C₁₂cycloalkyl, and 3- to 14-membered heterocyclyl containing 1-4 heteroatoms independently selected from the group consisting of O, N, and S,
wherein each alkyl or heterocyclyl is optionally substituted with one or more halogen, —O—C₁-C₆alkyl,—NH—C₁-C₆alkyl, —N(C₁-C₆alkyl)₂, —C₁-C₆alkyl optionally substituted with —OH, —C₃-C₁₂cycloalkyl, or 3- to 8-membered heterocyclyl containing 1-4 heteroatoms independently selected from the group consisting of O, N, and S.

Embodiment 47. The compound of any one of embodiments 1-46, wherein each R is independently selected from the group consisting of —H, —C₁-C₆alkyl, or 3- to 8-membered heterocyclyl optionally substituted with C₁-C₆alkyl.

Embodiment 48. The compound of any one of embodiments 1-47, wherein each R is —H or —C₁-C₆alkyl.

Embodiment 49. The compound of any one of embodiments 1-48, wherein each R′ is independently —C₁-C₆alkyl, —C₃-C₁₂cycloalkyl, or 3- to 14-membered heterocyclyl containing 1-4 heteroatoms independently selected from the group consisting of O, N, and S.

Embodiment 50. The compound of any one of embodiments 1-49, wherein each R′ is —C₁-C₆alkyl.

Embodiment 51. A compound selected from Table 1.

Embodiment 52. A compound of any one of embodiments 1-51, wherein the compound is a USP9X Inhibitor having an IC₅₀ value ≤2 μM in the assay of Example 1.

Embodiment 53. The compound of any one of embodiments 1-52, wherein the compound is a USP9X Inhibitor having an IC₅₀ value ≤0.2 μM in the assay of Example 1.

Embodiment 54. The compound of any one of embodiments 1-53, wherein the compound is a USP9X Inhibitor having an IC₅₀ value ≤0.05 μM in the assay of Example 1.

Embodiment 55. A pharmaceutical composition, comprising a compound of any one of embodiments 1-54, and a pharmaceutically acceptable carrier.

1. A compound of Formula I: 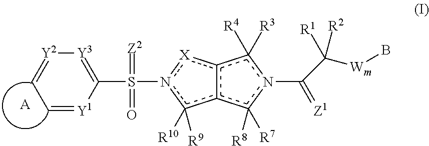
or a pharmaceutically acceptable salt thereof, wherein:
X is CR⁵R⁶, CR⁵, NR⁵, or N, as valency permits;
dashed bonds are each independently a single or a double bond, as valency permits,
wherein at least one dashed bond is a double bond;
Y¹, Y², and Y³ are each independently N or CR^{a};
each R^{a} is independently —H, halogen, or —CN;
Ring A is a 5- to 6-membered aryl, 5- to 6-membered heteroaryl containing 1-4 heteroatoms independently selected from the group consisting of O, N, and S, 5- to 7-membered heterocyclyl containing 1-4 heteroatoms independently selected from the group consisting of O, N, and S, or 5- to 7-membered cycloalkyl,
wherein each aryl, heteroaryl, heterocyclyl, or cycloalkyl is optionally substituted with one or more halogen, —C₁-C₆alkyl, —C₂-C₆alkenyl, —C₂-C₆alkynyl, oxo, or —C(O)R′;
Z¹ is O, S, or NR;
Z² is O or NR;
W is CR^{1′}R^{2′}, O, S, or NR;
m is 0 or 1;
R¹ and R² are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
wherein each cycloalkyl, cycloalkenyl, heterocyclyl, aryl, or heteroaryl is optionally substituted with one or more R^{e},
wherein each alkyl, alkenyl, or alkynyl is optionally substituted with one or more halogen,
wherein each heterocyclyl is 3- to 14-membered and contains 1-4 heteroatoms independently selected from the group consisting of O, N, and S, and wherein the heterocyclyl does not contain an O in the γ-position relative to C(═Z¹), and
wherein each heteroaryl is 5- to 14-membered and contains 1-4 heteroatoms independently selected from the group consisting of O, N, and S;
or R¹ and R² combine with the carbon to which they are attached to form oxo, a C₃-C₈cycloalkyl, or a 3- to 8-membered heterocyclyl containing 1-4 heteroatoms independently selected from the group consisting of O, N, and S, and wherein the heterocyclyl does not contain an O in the γ-position relative to C(═Z¹), and
wherein each cycloalkyl or heterocyclyl is optionally substituted with one or more R^{e};
R^{1′} and R^{2′} are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
wherein each alkyl, alkenyl, alkynyl, cycloalkyl, cycloalkenyl, heterocyclyl, aryl, or heteroaryl is optionally substituted with one or more R^{e},
wherein each heterocyclyl is 3- to 14-membered and contains 1-4 heteroatoms independently selected from the group consisting of O, N, and S, and
wherein each heteroaryl is 5- to 14-membered and contains 1-4 heteroatoms independently selected from the group consisting of O, N, and S;
or R^{1′} and R^{2′} combine with the carbon to which they are attached to form oxo, a C₃-C₈cycloalkyl, or a 3- to 8-membered heterocyclyl containing 1-4 heteroatoms independently selected from the group consisting of O, N and S,
wherein each heterocyclyl does not contain an O in the γ-position relative to C(═Z¹), and
wherein each cycloalkyl or heterocyclyl is optionally substituted with one or more R^{e};
or R¹ and R^{1′} combine with the carbons to which they are attached to form a C₃-C₈cycloalkyl or 3- to 8-membered heterocyclyl containing 1-4 heteroatoms independently selected from the group consisting of O, N and S,
wherein each heterocyclyl does not contain an O in the γ-position relative to C(═Z¹), and
wherein each cycloalkyl or heterocyclyl is optionally substituted with one or more R^{e};
R^{b} and R^{c} are each independently selected from the group consisting of —H, halogen, and —C₁-C₆alkyl;
each n is independently 0, 1, 2, 3, or 4;
each R^{e} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S, and
wherein —OR of R^{e} does not result in an O in the γ-position relative to C(═Z¹),
wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
B is a monocyclic or bicyclic 3- to 14-membered ring,
wherein the ring is saturated, fully or partially unsaturated, or aromatic, and
wherein the ring contains 0-4 heteroatoms independently selected from the group consisting of 0, N, and S,
wherein the ring is optionally substituted with one or more R^{d}, and
when m is 0 and the ring is saturated or partially unsaturated, then the ring does not contain an O in the γ-position relative to C(═Z¹);
each R^{d} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆ alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
each R³, R⁴, R⁵, R⁶, R⁷, R⁸, R⁹, and R¹⁰ is independently —H, —C₁-C₆alkyl, —C₃-C₈cycloalkyl, or 3- to 8-membered heterocyclyl containing 1-4 heteroatoms independently selected from the group consisting of O, N, and S,
wherein each alkyl, cycloalkyl, or heterocyclyl is optionally substituted with one or more halogen, oxo, —C₁-C₆alkyl, —C₂-C₆alkenyl, —C₂-C₆alkynyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₃-C₈cycloalkyl, or 3- to 8-membered heterocyclyl containing 1-4 heteroatoms independently selected from the group consisting of O, N, and S, and
wherein R³, R⁷, and R⁹ are each independently present or absent, as valency permits;
or R³ and R⁴, R⁵ and R⁶, R⁷ and R⁸, R⁹ and R¹⁰, or combinations thereof, combine with the carbon to which they are attached to form an oxo, C₃-C₈cycloalkyl, or 3- to 8-membered heterocyclyl containing 1-4 heteroatoms independently selected from the group consisting of O, N, and S;
each R is independently selected from the group consisting of —H, —OH, —O(C₁-C₆alkyl), —NH₂, —NH(C₁-C₆alkyl), —N(C₁-C₆alkyl)₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
wherein each alkyl, alkenyl, alkynyl, cycloalkyl, cycloalkenyl, heterocyclyl, aryl, or heteroaryl is optionally substituted with one or more halogen, oxo, —O—C₁-C₆alkyl, —NH(C₁-C₆alkyl), —N(C₁-C₆alkyl)₂, —C₁-C₆alkyl optionally substituted with one or more oxo or —OH, —C₂-C₆alkenyl, —C₂-C₆alkynyl, —C₃-C₁₂cycloalkyl, or 3- to 8-membered heterocyclyl containing 1-4 heteroatoms independently selected from the group consisting of O, N, and S; and
each R′ is independently selected from the group consisting of —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, aryl, and 5- to 14-membered heteroaryl containing 1-4 heteroatoms independently selected from the group consisting of O, N, and S,
wherein each alkyl, alkenyl, alkynyl, cycloalkyl, cycloalkenyl, heterocyclyl, aryl, or heteroaryl is optionally substituted with one or more halogen, oxo, —C₁-C₆alkyl optionally substituted with one or more oxo or —OH, —C₂-C₆alkenyl, —C₂-C₆alkynyl, —O—C₁-C₆alkyl, —NH(C₁-C₆alkyl), or —N(C₁-C₆alkyl)₂.

2. The compound of claim 1, or a pharmaceutically acceptable salt thereof, wherein m is 0.

3. The compound of claim 1, or a pharmaceutically acceptable salt thereof, wherein X is CR⁵R⁶, CR⁵, or N.

4. The compound of claim 1, or a pharmaceutically acceptable salt thereof, wherein Z¹ is O or S.

5. The compound of claim 1, or a pharmaceutically acceptable salt thereof, wherein Z² is O or NH.

6. The compound of claim 1, or a pharmaceutically acceptable salt thereof, wherein Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S, wherein each heteroaryl or heterocyclyl is optionally substituted with one or more halogen or —C₁-C₆alkyl.

7. The compound of claim 1, wherein the compound is of Formula II: 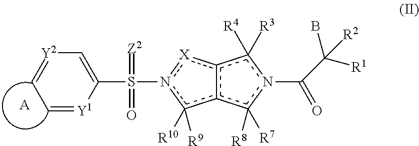
or a pharmaceutically acceptable salt thereof, wherein:
Ring A is a 5- to 6-membered heteroaryl containing 1-4 heteroatoms independently selected from the group consisting of O, N, and S, or a 5- to 6-membered heterocyclyl containing 1-4 heteroatoms independently selected from the group consisting of O, N, and S,
wherein each heteroaryl or heterocyclyl contains at least one oxygen atom and is optionally substituted with one or more halogen or —C₁-C₆alkyl.

8. The compound of claim 1, wherein the compound is of Formula II-c: 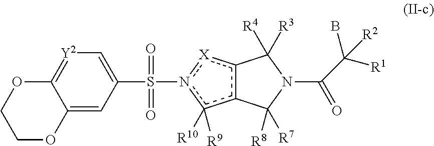
or a pharmaceutically acceptable salt thereof, wherein: Y² is CH or N.

9. The compound of claim 1, or a pharmaceutically acceptable salt thereof, wherein 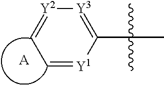
is selected from the group consisting of:

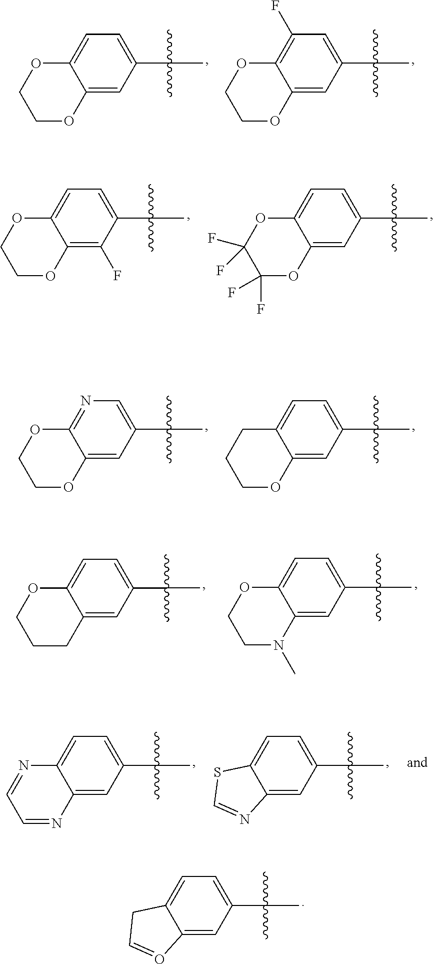

10. The compound of claim 1, or a pharmaceutically acceptable salt thereof, wherein

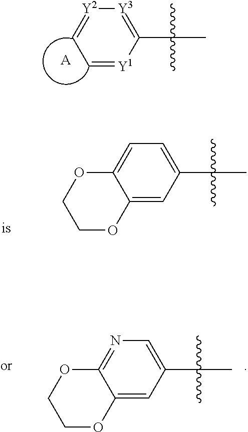

11. The compound of claim 1, or a pharmaceutically acceptable salt thereof, wherein B is a phenyl ring or a bicyclic ring,
wherein at least one of the rings in the bicyclic ring is a phenyl ring,
wherein the phenyl ring or bicyclic ring contains 0-4 heteroatoms independently selected from the group consisting of O, N, and S, and
wherein the phenyl ring or bicyclic ring is optionally substituted with one or more R^{d}.

12. The compound of claim 1, wherein the compound is: 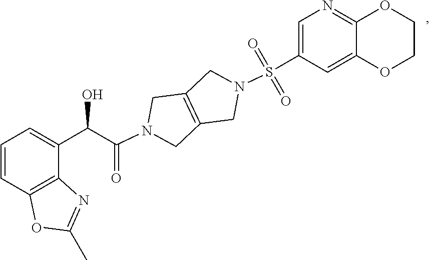 [SMILES: Cc1nc2c([C@@H](O)C(=O)N3CC4=C(C3)CN(S(=O)(=O)c3cnc5c(c3)OCCO5)C4)cccc2o1]
or a pharmaceutically acceptable salt thereof.

13. The compound of claim 1, wherein the compound is: 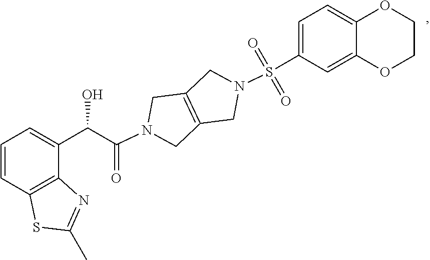 [SMILES: Cc1nc2c([C@H](O)C(=O)N3CC4=C(C3)CN(S(=O)(=O)c3ccc5c(c3)OCCO5)C4)cccc2s1]
or a pharmaceutically acceptable salt thereof.

14. The compound of claim 1, wherein the compound is: 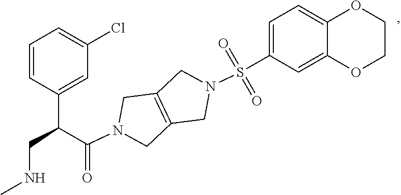 [SMILES: CNC[C@H](C(=O)N1CC2=C(C1)CN(S(=O)(=O)c1ccc3c(c1)OCCO3)C2)c1cccc(Cl)c1]
or a pharmaceutically acceptable salt thereof.

15. The compound of claim 1, wherein the compound is: 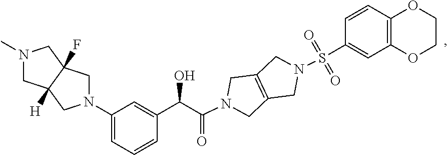 [SMILES: CN1C[C@H]2CN(c3cccc([C@@H](O)C(=O)N4CC5=C(C4)CN(S(=O)(=O)c4ccc6c(c4)OCCO6)C5)c3)C[C@@]2(F)C1]
or a pharmaceutically acceptable salt thereof.

16. The compound of claim 15, or a pharmaceutically acceptable salt thereof, wherein the compound is the second eluting isomer when separated by Chiral Prep-HPLC as in Table 21.

17.-29. (canceled)

30. The compound of claim 1, wherein the compound is: 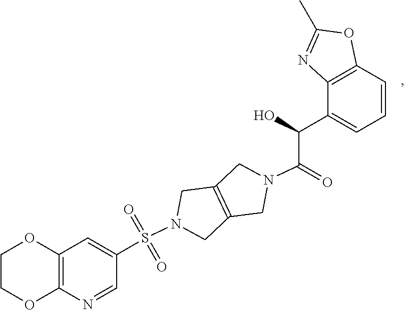 [SMILES: Cc1nc2c([C@H](O)C(=O)N3CC4=C(C3)CN(S(=O)(=O)c3cnc5c(c3)OCCO5)C4)cccc2o1]
or a pharmaceutically acceptable salt thereof.
